(19) United States
(12) Patent Application Publication
(10) Pub. No.: US 2026/0070876 A1
(43) Pub. Date: Mar. 12, 2026

(54) ONIUM SALT, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/285,183

(22) Filed: Jul. 30, 2025

(30) Foreign Application Priority Data
Aug. 8, 2024 (JP) 2024-132003

(51) Int. Cl. C07C 381/12; G03F 7/00; G03F 7/004; G03F 7/039

(52) U.S. Cl. CPC C07C 381/12; G03F 7/0045; G03F 7/0392; G03F 7/70033

(57) ABSTRACT

The onium salt consists of a cation having the formula (1A) and an anion having the formula (1B). The chemically amplified resist composition comprises the onium salt. The resist composition exhibits a high sensitivity, high resolution, improved lithography properties including EL, LWR, CDU and DOF, and collapse resistance, when processed by lithography using high-energy radiation such as deep UV, EB, or EUV.

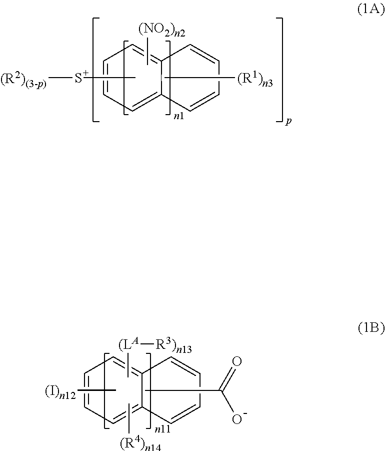

### CROSS-REFERENCE TO RELATED APPLICATION

This non-provisional application claims priority under 35 U.S.C. § 119(a) on Patent Application No. 2024-132003 filed in Japan on Aug. 8, 2024, the entire contents of which are hereby incorporated by reference.

### TECHNICAL FIELD

The invention relates to an onium salt, a chemically amplified resist composition, and a pattern forming process.

### BACKGROUND ART

To meet the demand for higher integration density and higher operating speed of LSIs, the effort to reduce the pattern rule is in rapid progress. As the use of 5G high-speed communications and artificial intelligence (AI) is widely spreading, high-performance devices are needed for their processing. As the most advanced miniaturization technology, mass production of microelectronic devices at the 5-nm node by the lithography using extreme ultraviolet (EUV) of wavelength 13.5 nm has been implemented. Studies are made on the application of EUV lithography to 3-nm node devices of the next generation and 2-nm node devices of the next-but-one generation.

As the feature size reduces, image blurs due to acid diffusion become a problem. To insure resolution for fine patterns with a size of 45 nm et seq., not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post exposure bake (PEB) fails, resulting in drastic reductions of sensitivity and contrast.

A triangular tradeoff relationship among sensitivity, resolution, and edge roughness (LWR) has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

Resist compositions adapted for the ArF lithography are typically based on (meth)acrylate polymers having acid labile groups. These acid labile groups undergo deprotection reaction when an acid generator capable of generating a sulfonic acid which is substituted at α-position with fluorine (referred to as “α-fluorinated sulfonic acid”, hereinafter) is used, but not when an acid generator capable of generating a sulfonic acid which is not substituted at α-position with fluorine (referred to as “α-non-fluorinated sulfonic acid”, hereinafter) or carboxylic acid is used. When a sulfonium or iodonium salt capable of generating α-fluorinated sulfonic acid is mixed with a sulfonium or iodonium salt capable of generating α-non-fluorinated sulfonic acid, the sulfonium or iodonium salt capable of generating α-non-fluorinated sulfonic acid undergoes ion exchange with the α-fluorinated sulfonic acid. Through the ion exchange, the α-fluorinated sulfonic acid once generated upon light exposure is converted back to the sulfonium or iodonium salt. Then the sulfonium or iodonium salt of α-non-fluorinated sulfonic acid or carboxylic acid functions as a quencher. Patent Document 3 discloses a resist composition comprising a sulfonium or iodonium salt capable of generating carboxylic acid as the quencher.

Sulfonium salt type quenchers capable of generating carboxylic acid are known. Proposed thus far are sulfonium salts of salicylic acid and β-hydroxycarboxylic acid (Patent Document 4), salicylic acid derivatives (Patent Documents 5 and 6), fluorosalicylic acids (Patent Document 7), hydroxynaphthoeic acid (Patent Document 8), salicylic acids having an iodized aromatic substituent group introduced therein (Patent Document 9), and salicylic acids having a cyclic acetal structure introduced therein (Patent Document 10). Salicylic acid is quite effective for suppressing acid diffusion due to the intramolecular hydrogen bond of carboxy group and hydroxy group.

It is pointed out that the agglomeration of a quencher causes to degrade the critical dimension uniformity (CDU) of resist patterns. It is thus expected that the critical dimension uniformity of resist patterns after development is improved by preventing the quencher from agglomerating in the resist film, for achieving a uniform distribution of the quencher.

In connection with the demand for further miniaturization, there is a problem that upon development of a positive resist film in an alkaline developer, the resist film is swollen with the developer so that pattern collapse may occur upon small-size pattern formation. To solve the problem associated with miniaturization, the development of an effective material for a new resist composition is important. It is desired to have an onium salt type quencher having a high sensitivity, fully controlled acid diffusion, acceptable solvent solubility, and effective prevention of pattern collapse.

### CITATION LIST

Patent Document 1: JP-A 2006-045311
Patent Document 2: JP-A 2006-178317
Patent Document 3: JP-A 2007-114431
Patent Document 4: WO 2018/159560
Patent Document 5: JP-A 2020-203984
Patent Document 6: JP-A 2020-091404
Patent Document 7: JP-A 2020-091312
Patent Document 8: JP-A 2019-120760
Patent Document 9: JP-A 2022-077505
Patent Document 10: WO 2023/189502
Non-Patent Document 1: SPIE Vol. 6520 65203L-1 (2007)

### SUMMARY OF THE INVENTION

In conjunction with the demand for higher resolution of resist patterns, a prior art resist composition comprising an acid generator of sulfonium salt type and a quencher fails to fully suppress acid diffusion. This raises the problem that lithography properties including contrast, exposure latitude (EL), LWR, CDU, and depth of focus (DOF) are degraded. Another problem arising upon formation of small-size patterns is pattern collapse by swell.

An object of the invention is to provide a novel onium salt and a chemically amplified resist composition comprising the onium salt, the resist composition exhibiting a high sensitivity, high resolution, improved lithography properties including EL, LWR, CDU and DOF, and collapse resistance, when processed by lithography using high-energy radiation such as deep UV, EB, or EUV. Another object is to provide a pattern forming process using the resist composition.

The inventor has found that an onium salt consisting of a sulfonium cation having a nitro group and an aromatic carboxylic acid anion containing at least one iodine atom on the aromatic ring has acceptable solvent solubility, and that a chemically amplified resist composition comprising the onium salt as a quencher exhibits a high sensitivity, high contrast, improved lithography properties including EL, LWR, CDU and DOF, and collapse resistance upon formation of small-size patterns.

That is, the invention provides the following onium salt, chemically amplified resist composition, and pattern forming process.
1. An onium salt consisting of a cation having the formula (1A) and an anion having the formula (1B):

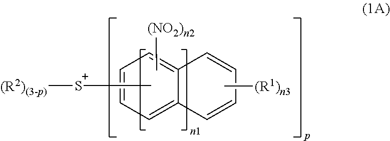

wherein p is 1, 2, or 3, n1 is 0 or 1, n2 is 1 or 2, n3 is 0, 1, 2, or 3, meeting 1 n2+n3≤5 in case of n1=0 and 1 n2+n3≤7 in case of n1=1,
   R¹ is halogen, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, and when n3 is 2 or 3, a plurality of R1 may be identical or different and two R¹ may bond together to form a ring with the carbon atoms to which they are attached,
   R² is halogen or a C₁-C₃₀ hydrocarbyl group which may contain a heteroatom, when p is 1, two R² may be identical or different, and any two of three substituents bonded to S⁺ may bond together to form a ring with the sulfur atoms to which they are attached;

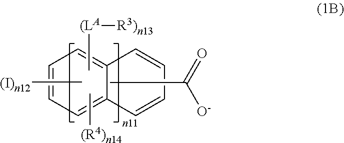

wherein n11 is 0 or 1, n12 is 1, 2, 3, or 4, n13 is 0, 1, or 2, n14 is 0, 1, 2, 3, or 4, meeting 2≤n12+n13+n14≤5 incase of n11=0 and 2≤n12+n13+n14≤7 incase of n11=1,
L^{A} is a single bond, oxygen, sulfur, ester bond or carbonate bond,
R³ is hydrogen or a C₁-C₂₀ hydrocarbyl group which may contain a heteroatom when L^{A} is a single bond, and hydrogen, a C₁-C₂₀ hydrocarbyl group (exclusive of acid labile group) which may contain a heteroatom, or acid labile group when L^{A} is oxygen, sulfur, ester bond or carbonate bond, and
R⁴ is halogen exclusive of iodine, a nitro group, cyano group, C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom.
2. The onium salt of 1 wherein the cation has the formula (1A-1):

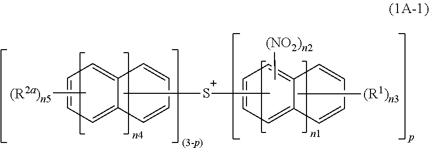

wherein p, n1 to n3, R¹, and Z⁻ are as defined above,
   n4 is 0 or 1, n5 is 0, 1, 2, 3, 4, or 5., and
   R^{2a} is halogen, cyano, hydroxy, carboxy, alkoxycarbonyl, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, and when n5 is 2, 3, 4, or 5, a plurality of R^{2a} may be identical or different and two R^{2a} may bond together to form a ring with the carbon atoms to which they are attached.
3. The onium salt of 1 or 2 wherein the anion has the formula (1B-1):

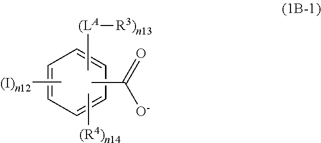

wherein n12 to n14, L^{A}, R³ and R⁴ are as defined above.
4. The onium salt of any one of 1 to 3 wherein L^{A} is oxygen or ester bond.
5. The onium salt of any one of 1 to 4 wherein L^{A} is oxygen, sulfur, ester bond or carbonate bond and R³ is an acid labile group.
6. The onium salt of any one of 1 to 5 wherein the acid labile group has the formula (AL-1) or (AL-2):

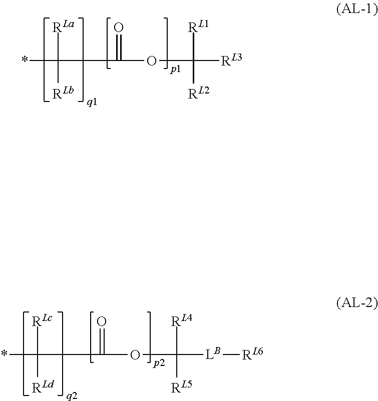

wherein p1 and p2 are each independently 0 or 1, q1 and q2 are each independently 0, 1, 2, 3, or 4,
   R^{L1}, R^{L2} and R^{L3} are each independently a C₁-C₁₂ hydrocarbyl group in which some —CH₂— may be replaced by —O— or —S—, when the hydrocarbyl group contains an aromatic ring, some or all of the hydrogen atoms in the aromatic ring may be substituted by halogen, cyano moiety, nitro moiety, optionally halogenated C₁-C₄ alkyl moiety, or optionally halogenated C₁-C₄ alkoxy moiety, R^{L1} and R^{L2} may bond together to form a ring with the carbon atom to which they are attached, some —CH₂— in the ring may be replaced by —O— or —S—,
   R^{L4} and R^{L5} are each independently hydrogen or a C₁-C₁₀ hydrocarbyl group, R^{L6} is a C₁-C₂₀ hydrocarbyl group in which some —CH₂— may be replaced by —O— or —S—, R^{L5} and R^{L6} may bond together to form a C₃-C₂₀ heterocyclic group with the carbon atom and L^{B} to which they are attached, some —CH₂— in the heterocyclic group may be replaced by —O— or —S—,
   L^{B} is —O— or —S—,
   R^{La} to R^{Ld} are each independently hydrogen or a C₁-C₂₀ hydrocarbyl group which may contain a heteroatom, and
   the asterisk (*) designates a point of attachment to the adjacent oxygen or sulfur.
7. A quencher in the form of the onium salt of any one of 1 to 6.
8. A chemically amplified resist composition comprising the quencher of 7.
9. The chemically amplified resist composition of 8, comprising a base polymer comprising repeat units having the formula (a1):

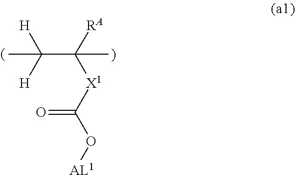

wherein R^{A} is hydrogen, fluorine, methyl or trifluoromethyl,
   X¹ is a single bond, phenylene group, naphthylene group, *—C(═O)—O—X¹¹— or *—C(═O)—NH—X¹¹—, the phenylene or naphthylene group may be substituted with an optionally fluorinated C₁-C₁₀ alkoxy moiety or halogen, X¹¹ is a C₁-C₁₀ saturated hydrocarbylene group which may contain a hydroxy moiety, ether bond, ester bond or lactone ring, or phenylene or naphthylene group, the asterisk (*) designates a point of attachment to the carbon atom in the backbone, and
   AL¹ is an acid labile group.
10. The chemically amplified resist composition of 9 wherein the base polymer comprises repeat units having the formula (a2):

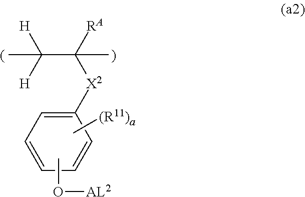

wherein R^{A} is hydrogen, fluorine, methyl or trifluoromethyl,
   X² is a single bond or *—C(═O)—O—, the asterisk (*) designates a point of attachment to the carbon atom in the backbone,
   R¹¹ is halogen, cyano, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R¹¹ may be identical or different when a1 is 2, 3, or 4,
   AL² is an acid labile group, and
   a is 0, 1, 2, 3, or 4.
11. The chemically amplified resist composition of 9 or 10 wherein the base polymer comprises repeat units having the formula (a3):

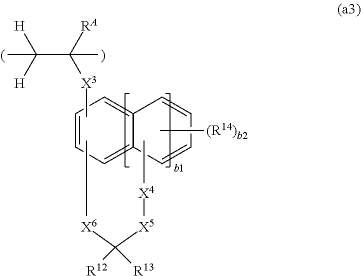

wherein b1 is 0 or 1, b2 is 0, 1, 2, or 3 in case of b1=0, and is 0, 1, 2, 3, 4, or 5 in case of b1=1,
   R^{A} is hydrogen, fluorine, methyl or trifluoromethyl,
   X³ is a single bond, *—C(═O)—O—, or *—C(═O)—NH—, the asterisk (*) designates a point of attachment to the carbon atom in the backbone,
   R¹² and R¹³ are each independently hydrogen or a C₁-C₂₀ hydrocarbyl group which may contain a heteroatom, R¹² and R¹³ may bond together to form a ring with the carbon atom to which they are attached,
   R¹⁴ is halogen, hydroxy, cyano, nitro, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group, a plurality of R¹⁴ may bond together to form a ring with the carbon atoms on the aromatic ring to which they are attached when b2 is 2 or more,
   X⁴ is a single bond, a C₁-C₄ aliphatic hydrocarbylene group, carbonyl group, sulfonyl group, or a group obtained by combining the foregoing,
   X⁵ and X⁶ are each independently oxygen or sulfur, X⁴ and X⁶ are bonded to vicinal carbon atoms on the aromatic ring.
12. The chemically amplified resist composition of any one of 9 to 11 wherein the base polymer comprises repeat units having the formula (bi) or (b2):

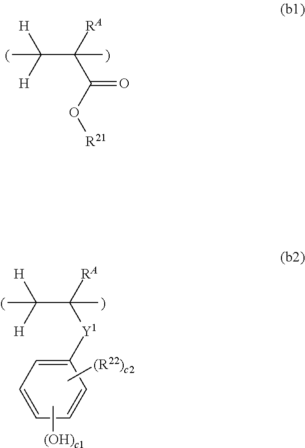

wherein R^{A} is each independently hydrogen, fluorine, methyl or trifluoromethyl,
   Y¹ is a single bond or *—C(═O)—O—, the asterisk (*) designates a point of attachment to the carbon atom in the backbone,
   R²¹ is hydrogen or a C₁-C₂₀ group containing at least one structure selected from among hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—),
   R²² is halogen, carboxy, nitro, cyano, alkoxycarbonyl, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R²² may be identical or different when c2 is 2, 3, or 4, and
   c1 is 1, 2, 3, or 4 and c2 is 0, 1, 2, 3, or 4, meeting 1≤c1+c2≤5.
13. The chemically amplified resist composition of any one of 9 to 12 wherein the base polymer comprises at least one selected from repeat units having the formula (c1), repeat units having the formula (c2), repeat units having the formula (c3), repeat units having the formula (c4), and repeat units having the formula (c5):

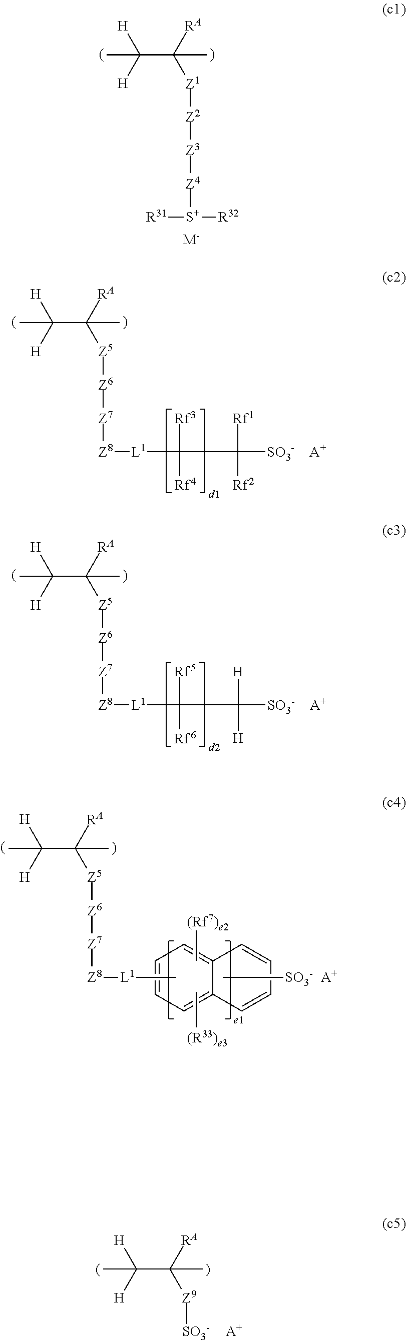

wherein d1 and d2 are each independently 0, 1, 2, or 3, e1 is 0 or 1, e2 is 0, 1, 2, 3, or 4, e3 is 0, 1, 2, 3, or 4, meeting 0≤e2+e3≤4 in case of e1=0 and 0≤e2+e3≤6 in case of e1=1,
   R^{A} is each independently hydrogen, fluorine, methyl or trifluoromethyl,
   Z¹ is a single bond or optionally substituted phenylene group, Z² is a single bond, **—C(═O)—O—Z²¹—, **—C(═O)—NH—Z²¹—, or **—O—Z²¹—, Z²¹ is a C₁-C₆ aliphatic hydrocarbylene group, phenylene group or a divalent group obtained by combining the foregoing, which may contain halogen, a carbonyl moiety, ester bond, ether bond or hydroxy moiety,
   Z³ is a single bond, ether bond, ester bond, amide bond, sulfonate ester bond, sulfonamide bond, carbonate bond or carbamate bond,
   Z⁴ is a single bond, or a C₁-C₆ aliphatic hydrocarbylene group, phenylene group or a divalent group obtained by combining the foregoing, which may contain halogen, carbonyl moiety, ester bond, ether bond or hydroxy moiety,
   Z⁵ is each independently a single bond, optionally substituted phenylene group, naphthylene group, or *—C(═O)—O—Z⁵¹, Z⁵¹ is a C₁-C₁₀ aliphatic hydrocarbylene group which may contain halogen, hydroxy moiety, ether bond, ester bond or lactone ring, or phenylene or naphthylene group,
   Z⁶ is a single bond, ether bond, ester bond, amide bond, sulfonate ester bond, sulfonamide bond, carbonate bond or carbamate bond,
   Z⁷ is each independently a single bond, ***—Z⁷¹—C(═O)—O—, ***—C(═O)—NH—Z⁷¹—, or ***—O—Z⁷—, Z⁷¹ is a C1-C₂₀ hydrocarbylene group which may contain a heteroatom,
   Z⁸ is each independently a single bond, ****-Z⁸¹-C(═O)—O—, ****—C(═O)—NH—Z⁸¹—, or ****—O—Z⁸¹—, Z⁸¹ is a C₁-C₂₀ hydrocarbylene group which may contain a heteroatom,
   Z⁹ is a single bond, methylene, ethylene, phenylene, fluorinated phenylene, trifluoromethyl-substituted phenylene, *—C(═O)—O—Z⁹¹—, *—C(═O)—N(H)—Z⁹¹—, or *—O—Z⁹¹—, Z⁹¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety,
   the asterisk (*) designates a point of attachment to the carbon atom in the backbone, the asterisks (**) designate a point of attachment to Z¹, the asterisks (***) designate a point of attachment to Z⁶, the asterisks (****) designate a point of attachment to Z⁷,
   L¹ is a single bond, ether bond, ester bond, carbonyl group, sulfonate ester bond, carbonate bond or carbamate bond,
   Rf¹ and Rf² are each independently fluorine or a C₁-C₆ fluorinated saturated hydrocarbyl group,
   Rf³ and Rf⁴ are each independently hydrogen, fluorine, or a C₁-C₆ fluorinated saturated hydrocarbyl group,
   Rf⁵ and Rf⁶ are each independently hydrogen, fluorine, or a C₁-C₆ fluorinated saturated hydrocarbyl group, excluding that all Rf⁵ and Rf⁶ are hydrogen at the same time,
   Rf⁷ is fluorine, a C₁-C₆ fluorinated alkyl group, a C₁-C₆ fluorinated alkoxy group, or a C₁-C₆ fluorinated alkylthio group,
   R³¹ and R³² are each independently a C₁-C₂₀ hydrocarbyl group which may contain a heteroatom, R³¹ and R³² may bond together to form a ring with the sulfur atom to which they are attached,
   R³³ is halogen exclusive of fluorine, or a C₁-C₂₀ hydrocarbyl group which may contain a heteroatom, when e3 is 2, 3, or 4, a plurality of R³³ may be identical or different and a plurality of R³³ may bond together to form a ring with the carbon atoms to which they are attached,
   M⁻ is a non-nucleophilic counter ion, and
   A⁺ is an onium cation.
14. The chemically amplified resist composition of any one of 8 to 13, further comprising an organic solvent.
15. The chemically amplified resist composition of any one of 8 to 14, further comprising a photoacid generator capable of generating a strong acid.
16. The chemically amplified resist composition of any one of 8 to 15, further comprising a quencher other than the quencher of 7.
17. The chemically amplified resist composition of any one of 8 to 16, further comprising a surfactant.
18. A pattern forming process comprising the steps of applying the chemically amplified resist composition of any one of 8 to 17 onto a substrate to form a resist film thereon, exposing the resist film to high-energy radiation, and developing the exposed resist film in a developer.
19. The pattern forming process of 18 wherein the high-energy radiation is KrF excimer laser, ArF excimer laser, EB, or EUV of wavelength 3 to 15 nm.

### Advantageous Effects of the Invention

When pattern is formed using a chemically amplified resist composition comprising the onium salt of the invention as a quencher, it is possible to form a resist pattern having a high contrast, a good sensitivity, and improved lithography properties such as EL, LWR, CDU, and DOF.

### DESCRIPTION OF THE PREFERRED EMBODIMENT

### Onium Salt

The onium salt of the invention comprises a cation having the formula (1A).

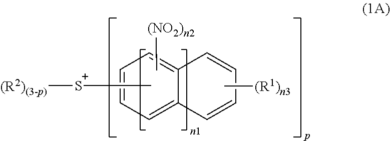

In the formula (1A), p is 1, 2, or 3.

In the formula (1A), n1 is 0 or 1. The relevant structure is a benzene ring in case of n1=0, and a naphthalene ring in case of n1=1. The benzene ring corresponding to n1=0 is preferred from the aspect of solvent solubility. n2 is 1 or 2. n2 is preferably 1 from the aspect of availability of reactants. n3 is 0, 1, 2, or 3. n3 is preferably 0, 1 or 2 from the aspect of availability of reactants. These subscripts meet 1≤n2+n3≤5 in case of n1=0 and 1 n2+n3 7 in case of n1=1.

In the formula (A), R¹ is halogen, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. Examples of the halogen include fluorine, chlorine, bromine, and iodine. The hydrocarbyl group and hydrocarbyl moiety in the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl; and combinations thereof. Of these, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. A plurality of R¹ may be identical or different when n3 is 2 or 3. Also, when n3 is 2 or 3, a plurality of R¹ may bond together to form a ring with the carbon atoms to which they are attached. The ring is preferably a 5- to 8-membered ring.

In the formula (1A), R² is halogen or a C₁-C₃₀ hydrocarbyl group which may contain a heteroatom. When p is 1, two R² may be identical or different.

Examples of the halogen represented by R² include fluorine, chlorine, bromine, and iodine.

The hydrocarbyl group represented by R² may be saturated or unsaturated and straight, branched or cyclic. Examples thereof include C₁-C₃₀ alkyl groups such as methyl, ethyl, n-propyl, isopropyl, n-butyl, isobutyl, sec-butyl, and tert-butyl; C₃-C₃₀ cyclic saturated hydrocarbyl groups such as cyclopropyl, cyclopentyl, cyclohexyl, cylopropylmethyl, 4-methylcyclohexyl, cyclohexylmethyl, norbornyl, and adamantyl; C₂-C₃₀ alkenyl groups such as vinyl, 1-propenyl, 2-propenyl, butenyl, and hexenyl; C₃-C₃₀ cyclic unsaturated hydrocarbyl groups such as cyclohexenyl; C₆-C₃₀ aryl groups such as phenyl, naphthyl, and thienyl; C₇-C₃₀ aralkyl groups such as benzyl, 1-phenylethyl, and 2-phenylethyl; and combinations thereof. Of these, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, nitro moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

Any two of three substituents bonded to S⁺ may bond together to form a ring with the sulfur atoms to which they are attached. Examples of the structure of the ring include structures having the formulae:

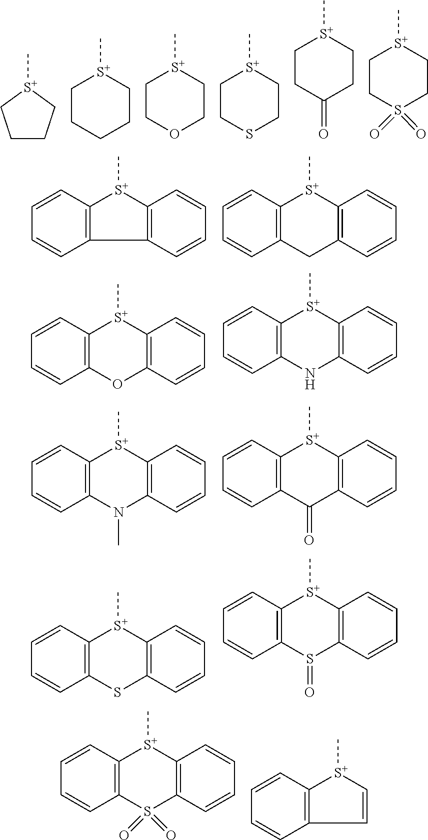

wherein the broken line is a valence bond.

As the cation having the formula (1A), a cation having the formula (1A-1) is preferred.

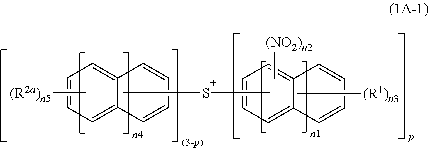

Herein p, n1 to n3, and R¹ are as defined above, and

In the formula (1A-1), n4 is 0 or 1. The relevant structure is a benzene ring in case of n4=0, and a naphthalene ring in case of n4=1. The benzene ring corresponding to n4=0 is preferred from the aspect of solvent solubility. n5 is 0, 1, 2, 3, 4, or 5. n5 is preferably 0, 1 or 2 from the aspect of availability of reactants.

In the formula (1A-1), R^{2a} is halogen, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group, or a C₂-C₂₀ hydrocarbyloxycarbonyl group which may contain a heteroatom. The hydrocarbyl group and hydrocarbyl moiety in the hydrocarbyloxy and hydrocarbylthio groups may be saturated or unsaturated and straight, branched or cyclic. Examples thereof include those exemplified above for the hydrocarbyl group represented by R¹, but not limited thereto. When n5 is 2, 3, 4, or 5, a plurality of R^{2a} may be identical or different and two R^{2a} may bond together to form a ring with the carbon atoms on the aromatic ring to which they are attached.

Examples of the cation having the formula (1A) include those shown below, but not limited thereto.

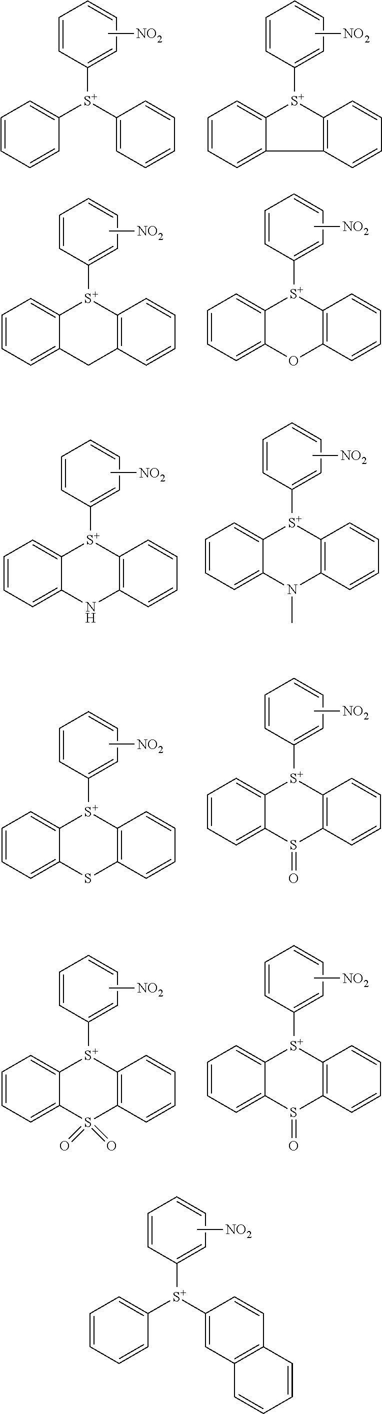

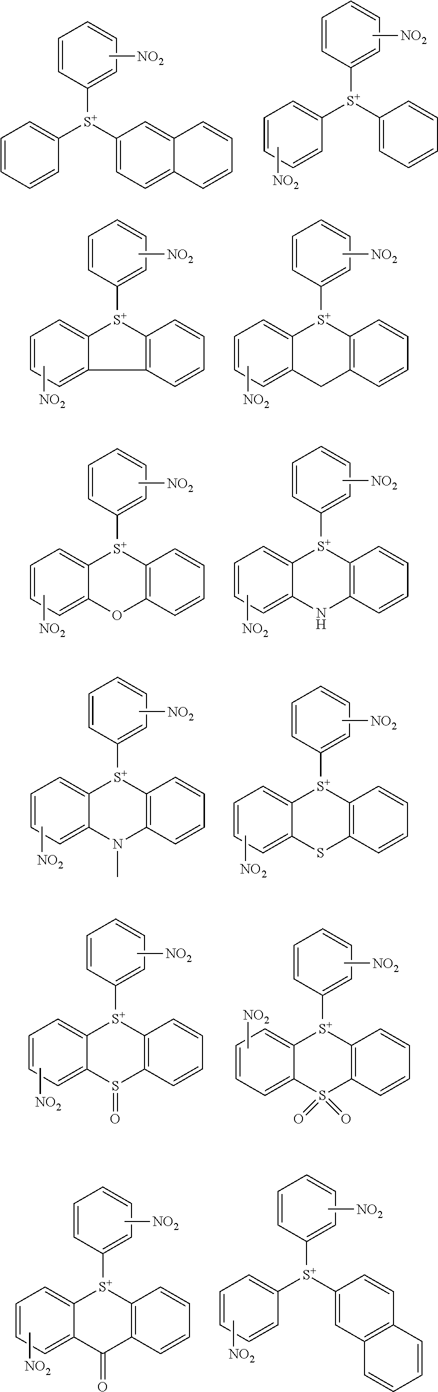

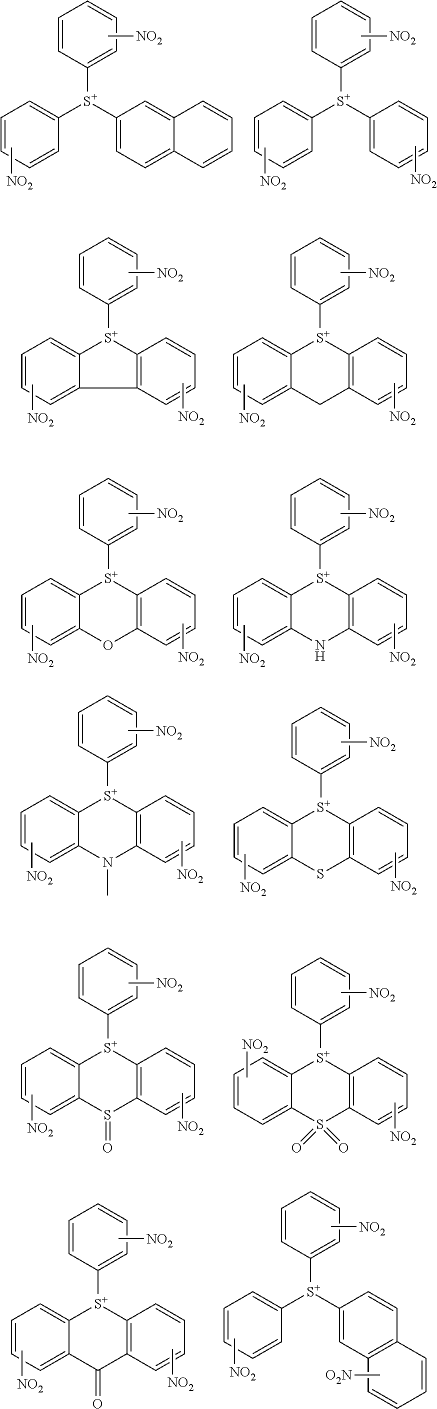

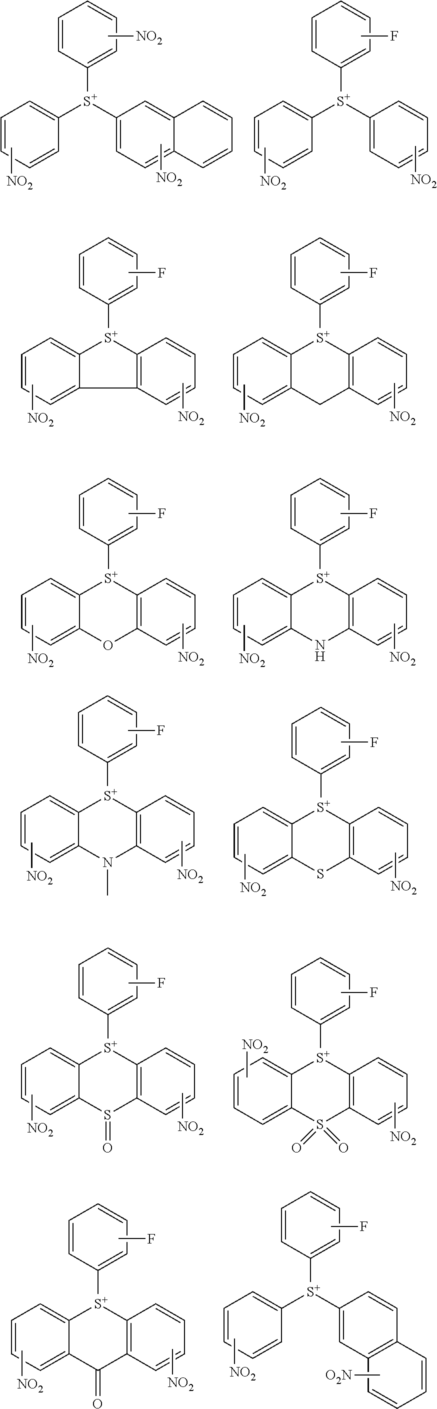

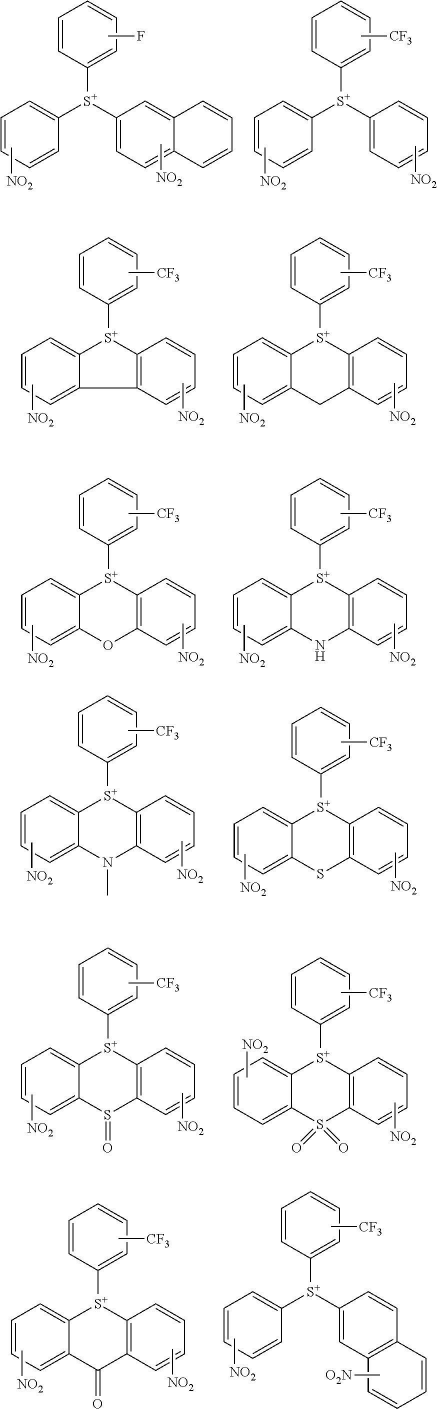

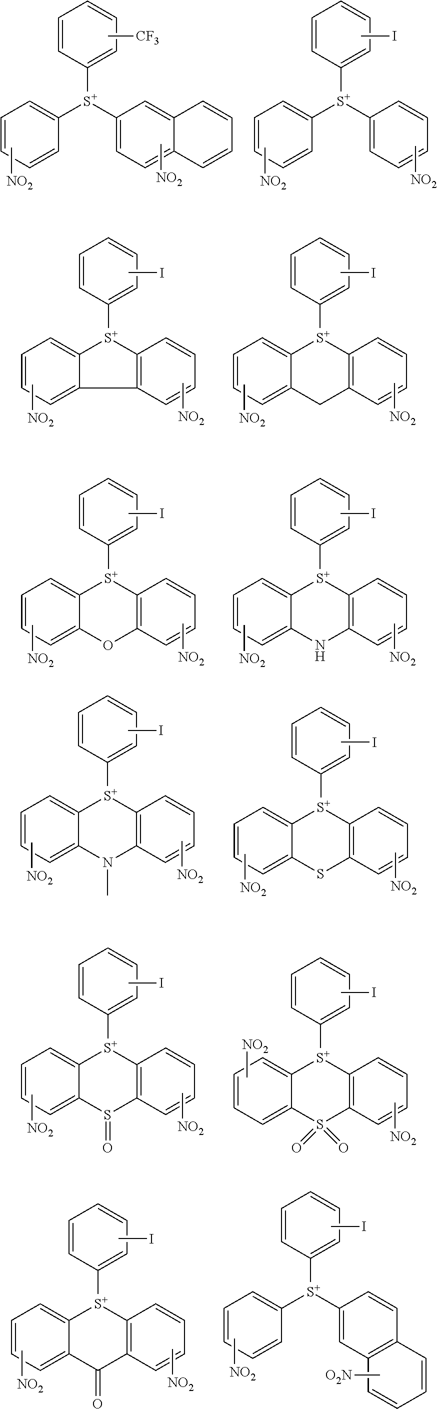

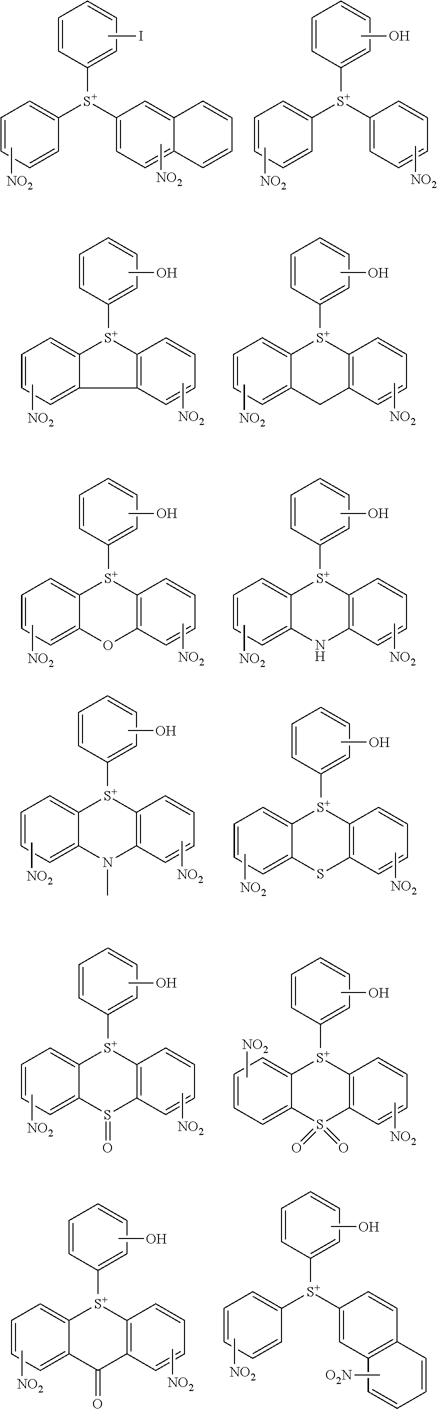

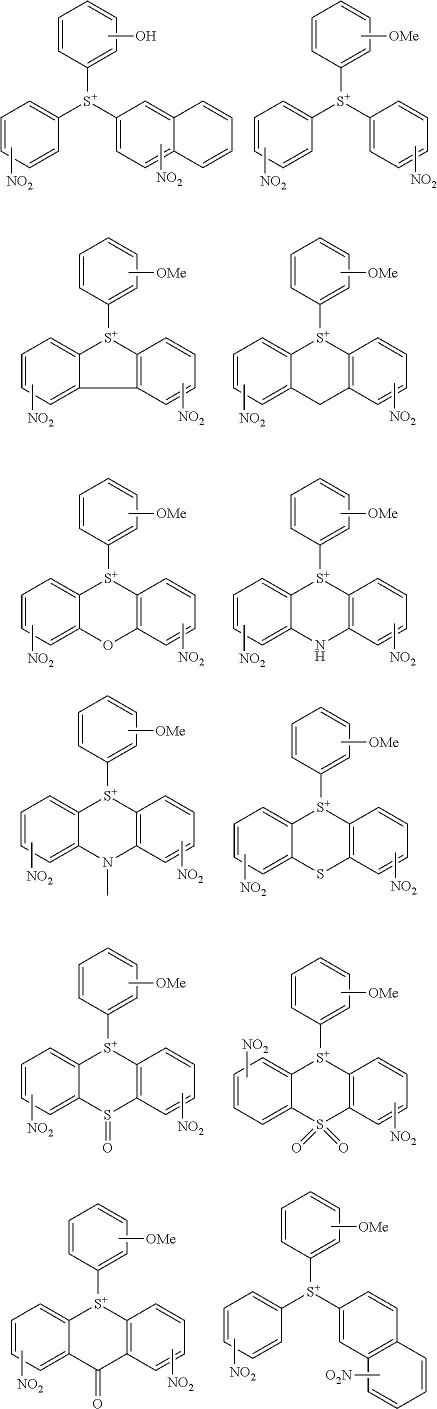

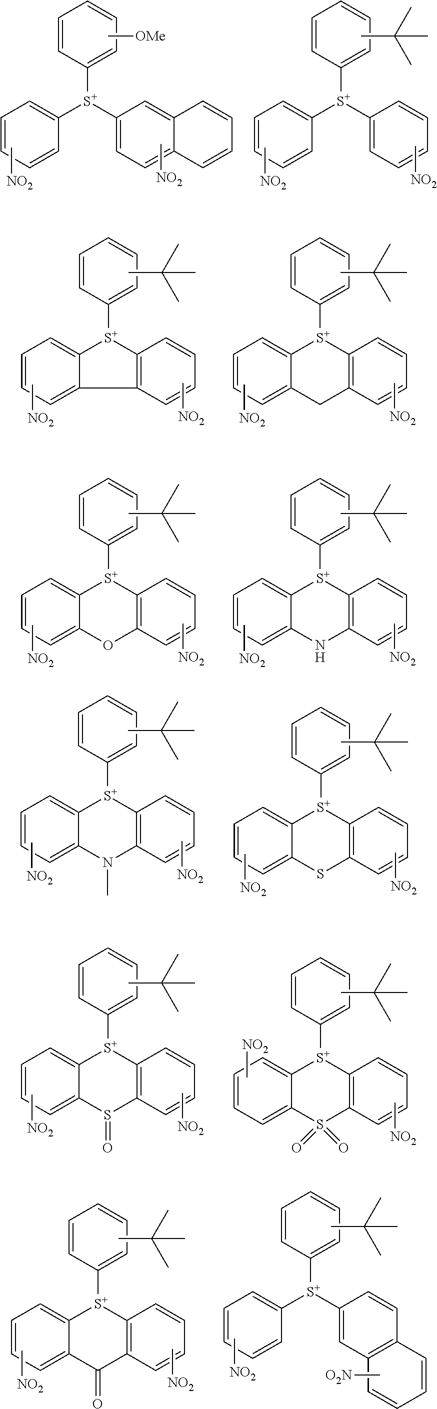

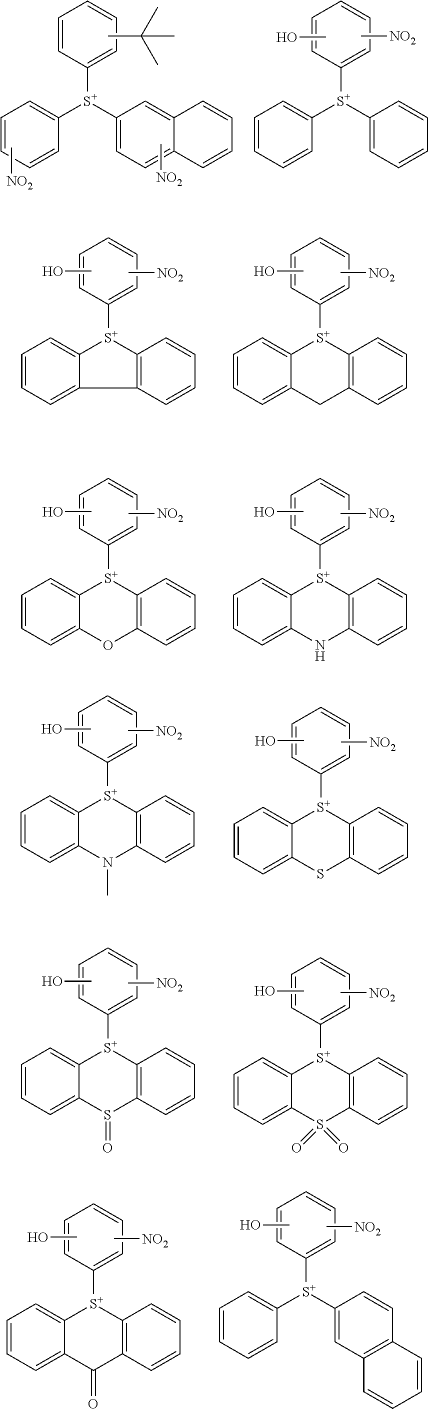

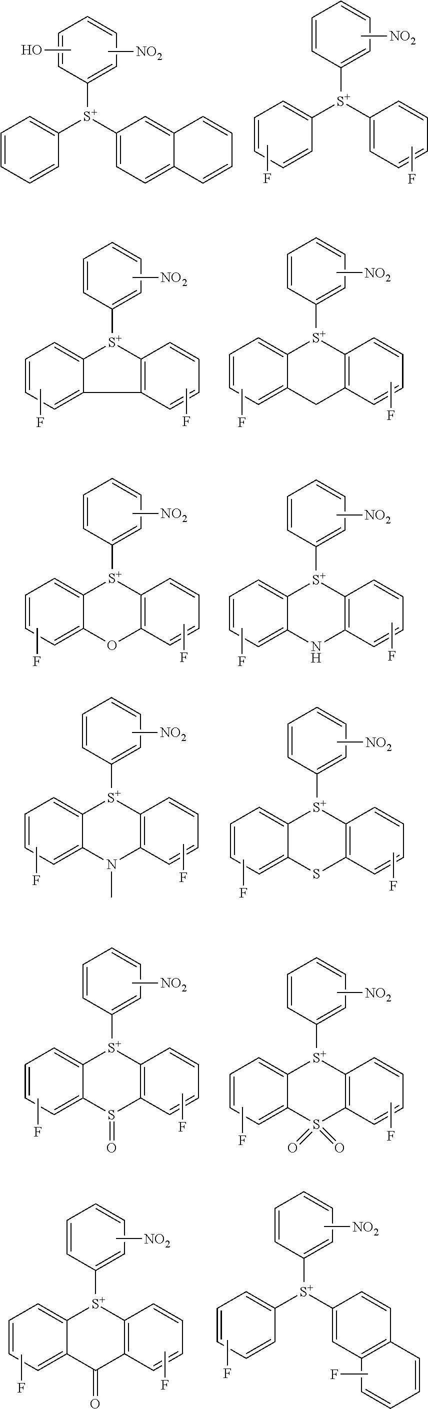

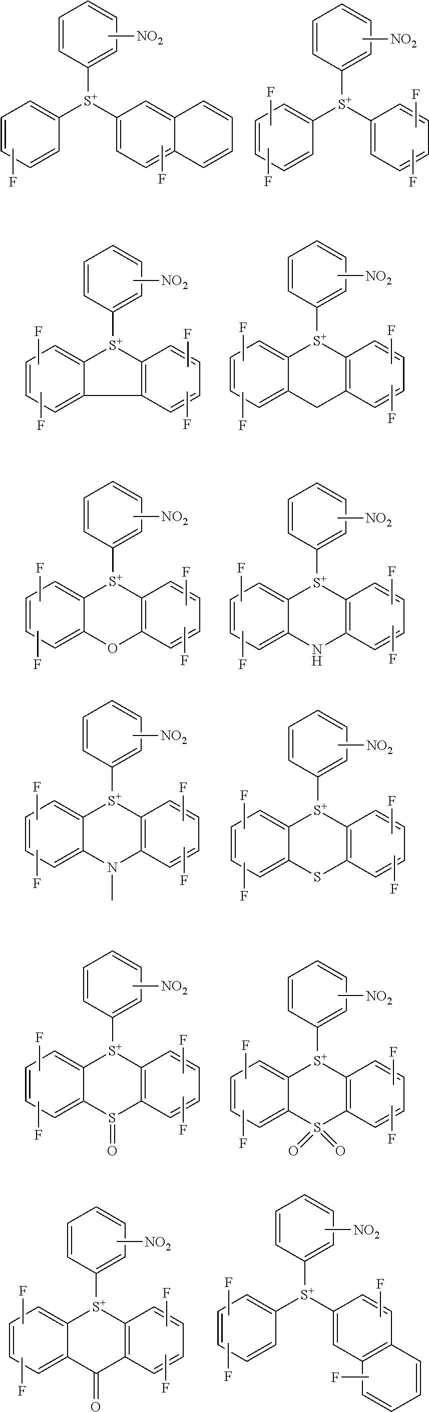

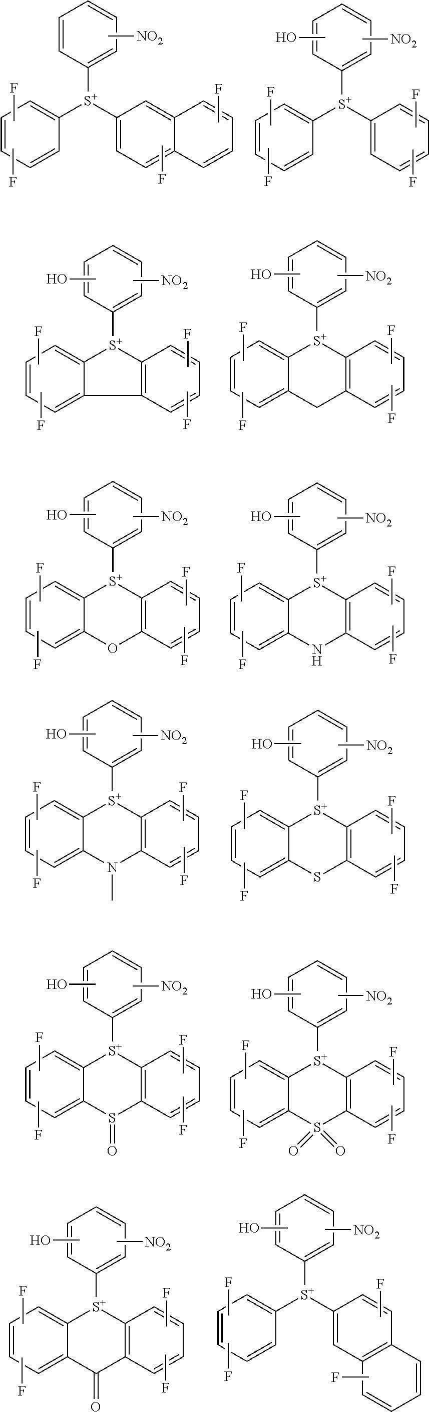

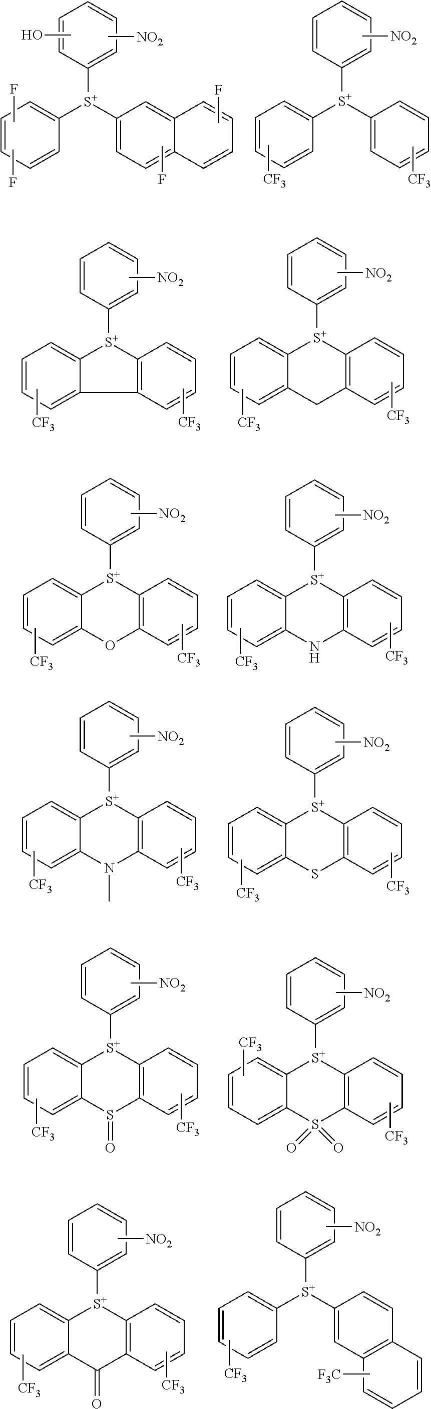

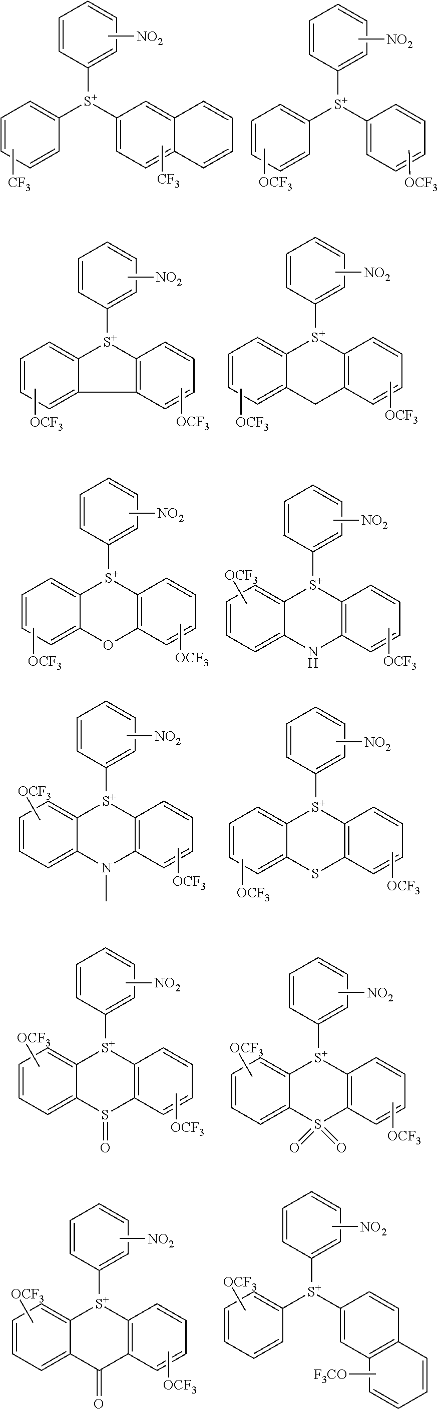

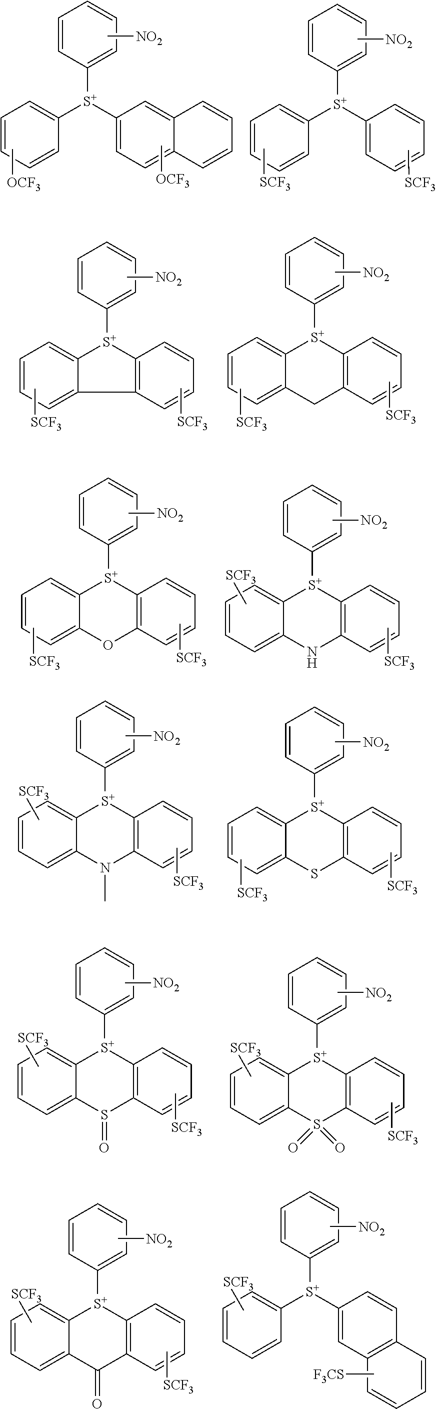

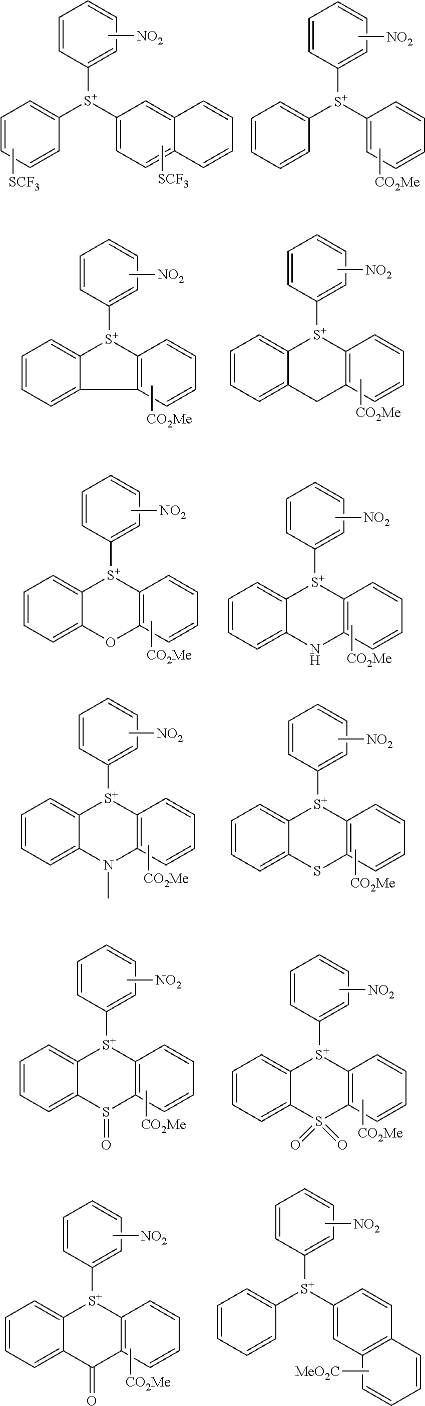

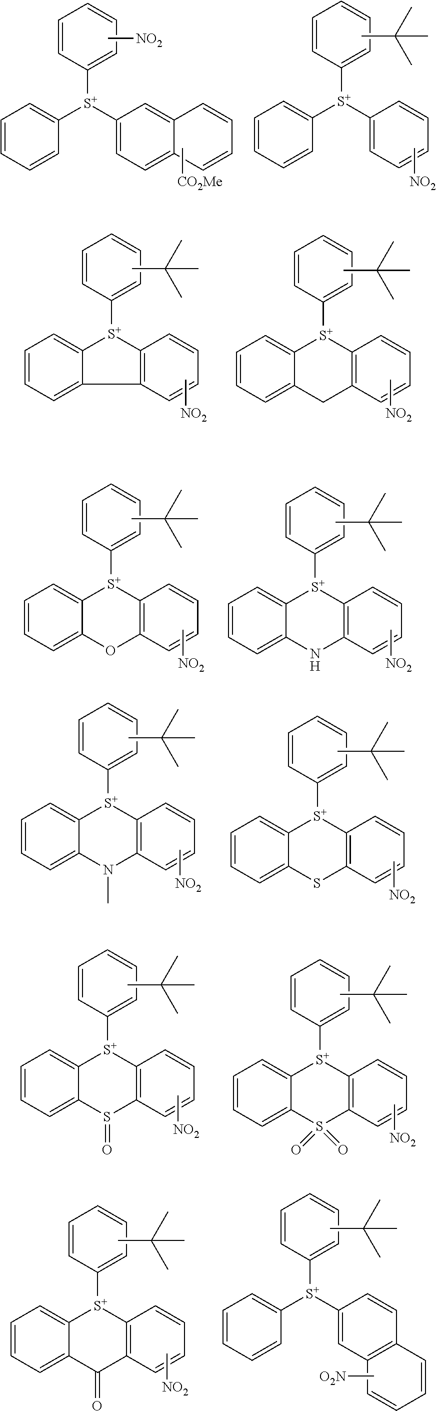

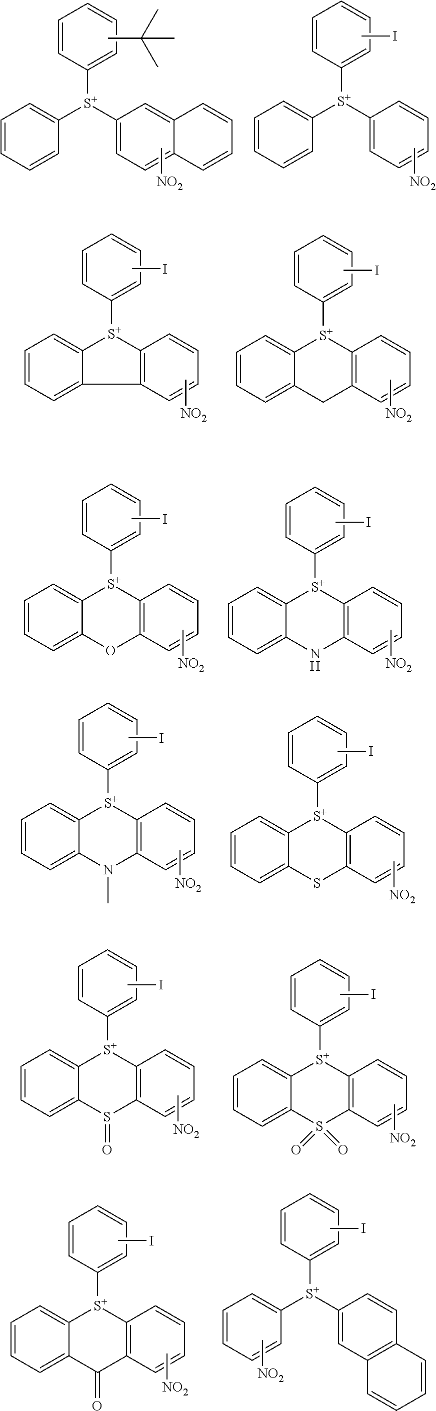

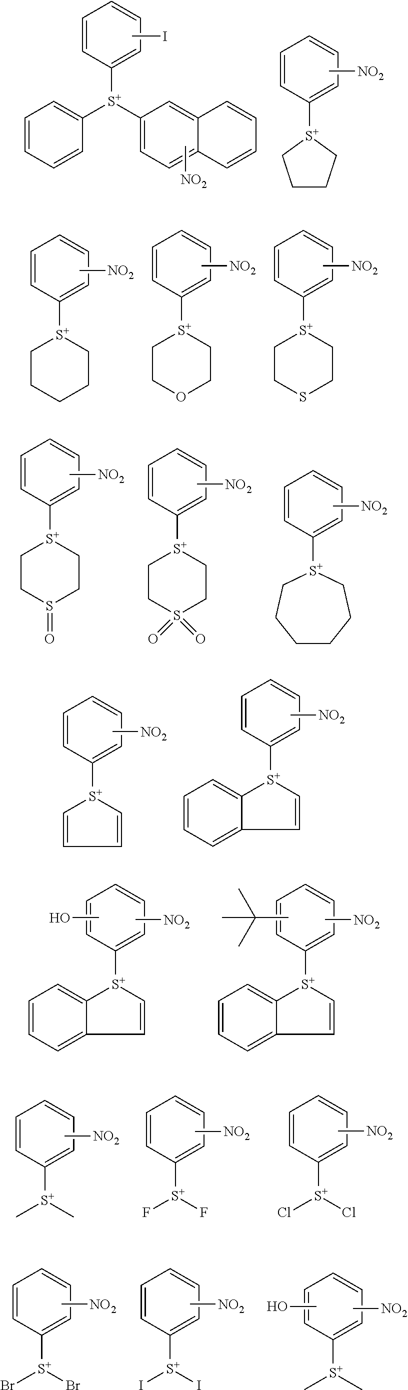

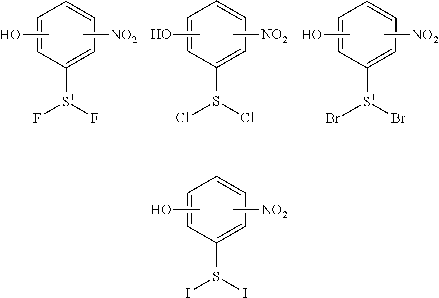

The onium salt of the invention comprises a carboxylic acid anion having the formula (1B).

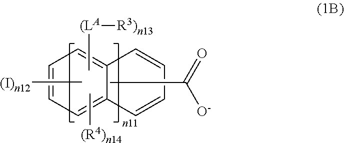

In the formula (1B), n11 is 0 or 1. The relevant structure is a benzene ring in case of n11=0, and a naphthalene ring in case of n11=1. The benzene ring corresponding to n11=0 is preferred from the aspect of solvent solubility. n12 is 1, 2, 3, or 4, and is preferably 1, 2, or 3 from the aspect of availability of reactants, more preferably 1 or 2 from the aspect of solvent solubility. n13 is 0, 1, or 2. n14 is 0, 1, 2, 3, or 4. 2≤n12+n13+n14≤5 incase of n11=0 and 2≤n12+n13+n14≤7 incase of n11=1. n14 is preferably 0, 1, 2, or 3, more preferably 0, 1, or 2 from the aspect of availability of reactants.

In the formula (1B), L^{A} is a single bond, oxygen, sulfur, ester bond or carbonate bond. When R³ is hydrogen, L^{A} is preferably oxygen. When R³ is an acid labile group, R³ is preferably oxygen, ester bond or carbonate bond, more preferably oxygen or ester bond.

In the formula (1B), when L^{A} is a single bond, R³ is hydrogen or a C₁-C₂₀ hydrocarbyl group which may contain a heteroatom. The C₁-C₂₀ hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, n-decyl, n-undecyl, and n-dodecyl; C₃-C₂₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, norbornylmethyl, adamantyl, adamantylmethyl, tricyclo[5.2.1.0^{2,6}]decyl, and tetracyclo[6.2.1.1^{3,6}.0^{2,7}]dodecyl; C₂-C₂₀ alkenyl groups such as vinyl, allyl, propenyl, butenyl, pentenyl, and hexenyl; C₂-C₂₀ alkynyl groups such as ethynyl, propynyl, butynyl, pentynyl, and hexynyl; C₃-C₂₀ cyclic unsaturated aliphatic hydrocarbyl groups such as cyclopentenyl and cyclohexenyl; C6-C₂₀ aryl groups such as phenyl, naphthyl, and indanyl; C₇-C₂₀ aralkyl groups such as benzyl, 1-phenylethyl, and 2-phenylethyl; and combinations thereof. The hydrocarbyl group may contain a heteroatom such as halogen, oxygen or sulfur.

When L^{A} is oxygen, sulfur, ester bond or carbonate bond, R³ is hydrogen, a C₁-C₂₀ hydrocarbyl group (exclusive of acid labile group) which may contain a heteroatom, or acid labile group. Examples of the hydrocarbyl group are as exemplified above, with acid labile groups being excluded.

Typical of the acid labile group are groups having the formulae (AL-1) and (AL-2):

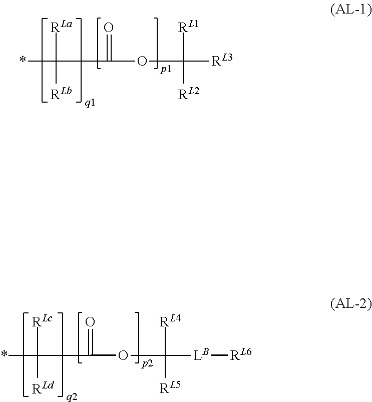

wherein the asterisk (*) designates a point of attachment to the adjacent oxygen or sulfur.

In the formulae (AL-1) and (AL-2), p1 and p2 are each independently 0 or 1. q1 and q2 are each independently 0, 1, 2, 3, or 4. When L^{A} is oxygen with which R³ forms a carbonyl group, p1 or p2 is 1. Otherwise, p1 or p2 is preferably 0. When p1=1, q1 is preferably 1 or 2, and when p1=0, q1 is 0. When p2=1, q2 is preferably 1 or 2, and when p2=0, q2 is 0.

In the formula (AL-1), R^{L1}, R^{L2} and R^{L3} are each independently a C₁-C₁₂ hydrocarbyl group in which some —CH₂— may be replaced by —O— or —S—, when the hydrocarbyl group contains an aromatic ring, some or all of the hydrogen atoms in the aromatic ring may be substituted by halogen, cyano moiety, nitro moiety, optionally halogenated C₁-C₄ alkyl moiety, or optionally halogenated C₁-C₄ alkoxy moiety.

The C₁-C₁₂ hydrocarbyl groups represented by R^{L1}, R^{L2} and R^{L3} may be saturated or unsaturated and straight, branched or cyclic. Examples thereof include C₁-C₁₂ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, n-decyl, n-undecyl, and n-dodecyl; C₃-C₁₂ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, norbornylmethyl, adamantyl, adamantylmethyl, tricyclo[5.2.1.0^{2,6}]decyl, and tetracyclo[6.2.1.1^{3,6}.0^{2,7}]dodecyl; C₂-C₁₂ alkenyl groups such as vinyl, allyl, propenyl, butenyl, pentenyl, and hexenyl; C₂-C₁₂ alkynyl groups such as ethynyl, propynyl, butynyl, pentynyl, and hexynyl; C₃-C₁₂ cyclic unsaturated aliphatic hydrocarbyl groups such as cyclopentenyl and cyclohexenyl; C₆-C₁₂ aryl groups such as phenyl, naphthyl, and indanyl; C₇-C₁₂ aralkyl groups such as benzyl, 1-phenylethyl, and 2-phenylethyl; and combinations thereof.

R^{L1} and R^{L2} may bond together to form a ring with the carbon atom to which they are attached, some —CH₂— in the ring may be replaced by —O— or —S—. Exemplary rings include cyclopropane, cyclobutane, cyclopentane, cyclohexane, cycloheptane, cyclooctane, norbornane, adamantane, tricyclo[5.2.1.0^{2,6}]decane, and tetracyclo[6.2.1.1^{3,6}.0^{2,7}]dodecane rings as well as these rings in which some —CH₂— may be replaced by —O— or —S—.

In the formula (AL-2), R^{L4} and R^{L5} are each independently hydrogen or a C1-C₁₀ hydrocarbyl group. The C₁-C₁₀ hydrocarbyl groups represented by R^{L4} and R^{L5} may be saturated or unsaturated and straight, branched or cyclic. Examples thereof include those exemplified above for the hydrocarbyl groups represented by R^{L1}, R^{L2} and R^{L3}, but of 1 to 10 carbon atoms.

In the formula (AL-2), R^{L6} is a C₁-C₂₀ hydrocarbyl group in which some —CH₂— may be replaced by —O— or —S—. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cylopropylmethyl, 4-methylcyclohexyl, cyclohexylmethyl, norbornyl, norbornylmethyl, adamantyl, adamantylmethyl, tricyclo[5.2.1.0^{2,6}]decyl, and tetracyclo[6.2.1.1^{3,6}.0^{2,7}]dodecyl; C₂-C₂₀ alkenyl groups such as vinyl, propenyl, butenyl, pentenyl, and hexenyl; C₂-C₂₀ alkynyl groups such as ethynyl, propynyl, butynyl, pentynyl, and hexynyl; C₃-C₂₀ cyclic unsaturated aliphatic hydrocarbyl groups such as cyclopentenyl, cyclohexenyl, and norbornenyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl; C₇-C₂₀ aralkyl groups such as benzyl and phenethyl; and combinations thereof. R^{L5} and R^{L6} may bond together to form a C₃-C₂₀ heterocyclic group with the carbon atom and L^{B} to which they are attached, some —CH₂— in the heterocyclic group may be replaced by —O— or —S—,

In the formula (AL-2), L^{B} is —O— or —S—.

In the formulae (AL-1) and (AL-2), R^{La} to R^{Ld} are each independently hydrogen or a C₁-C₂₀ hydrocarbyl group which may contain a heteroatom. The C₁-C₂₀ hydrocarbyl group may be saturated or unsaturated and straight, branched or cyclic. Examples thereof include those exemplified above for the hydrocarbyl group represented by R³, but not limited thereto.

Examples of the acid labile group having the formula (AL-1) include those shown below, but not limited thereto. In the formulae, the asterisk (*) designates a point of attachment to the adjacent oxygen or sulfur.

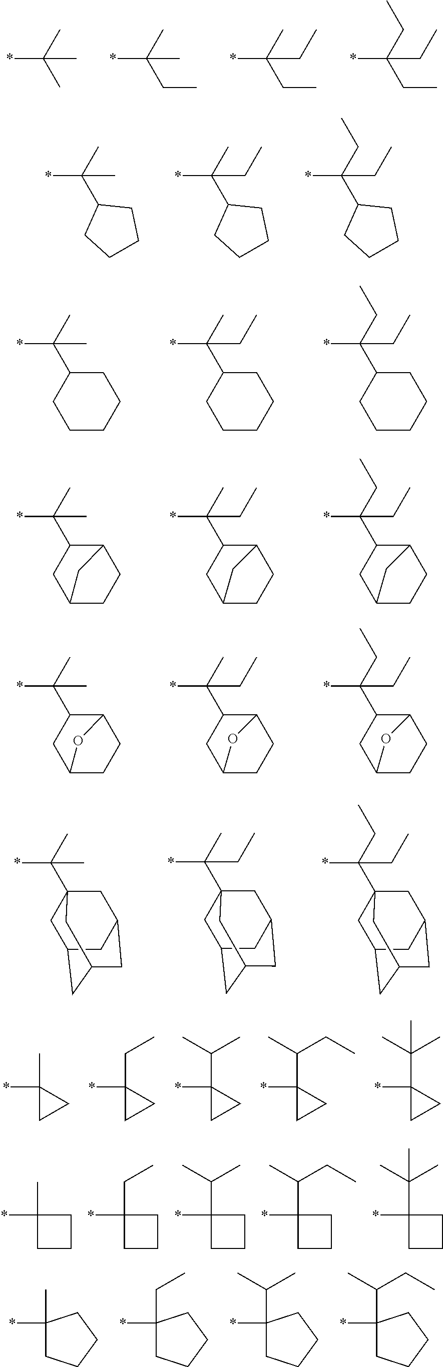

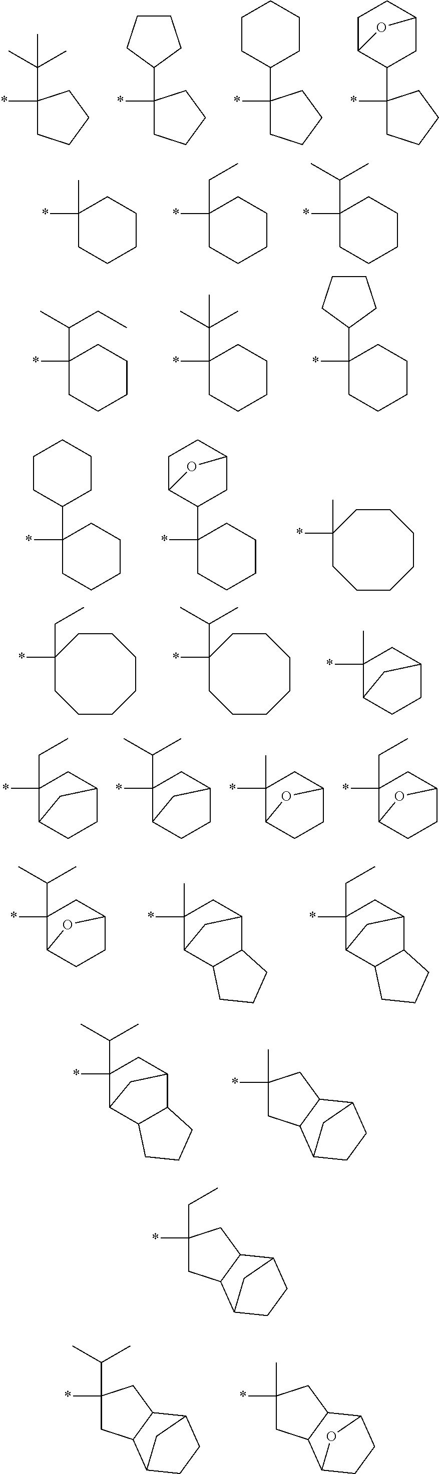

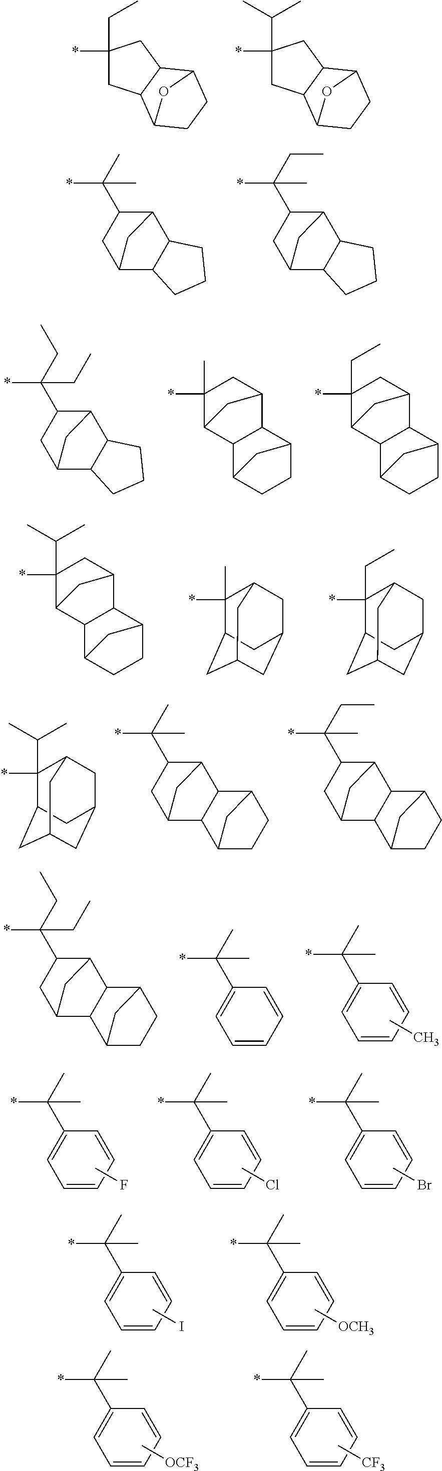

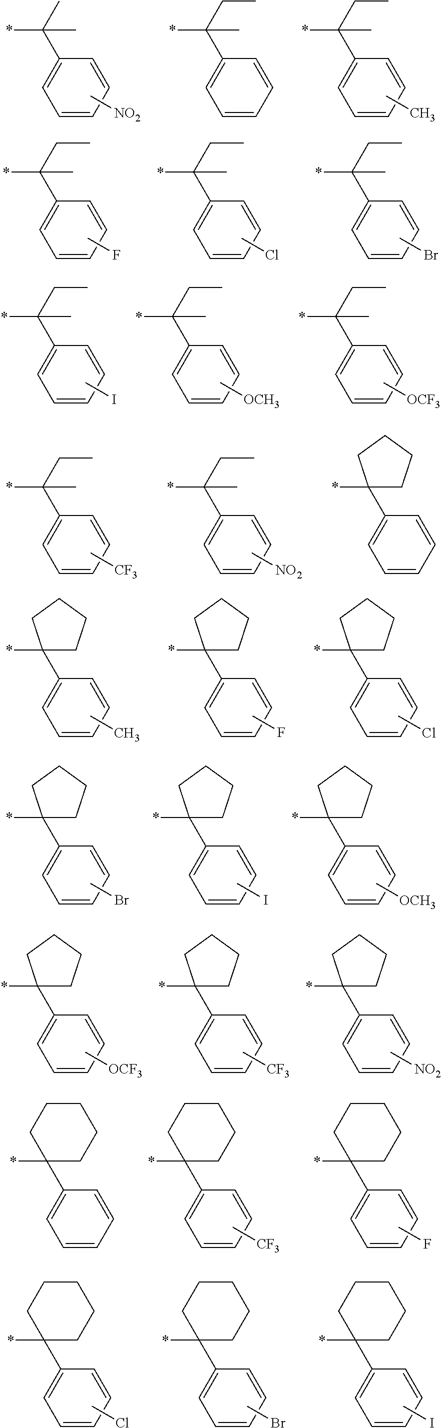

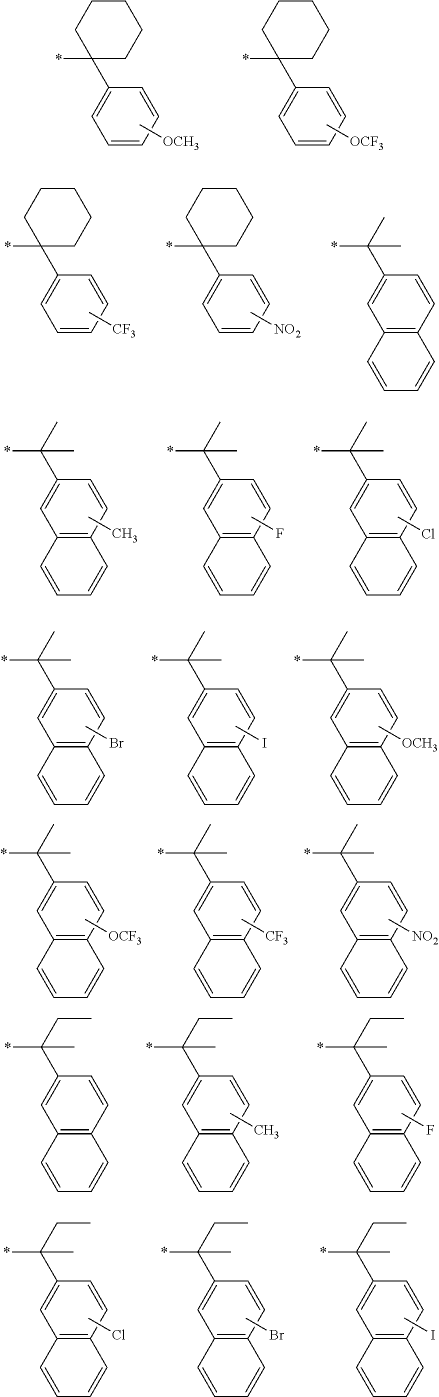

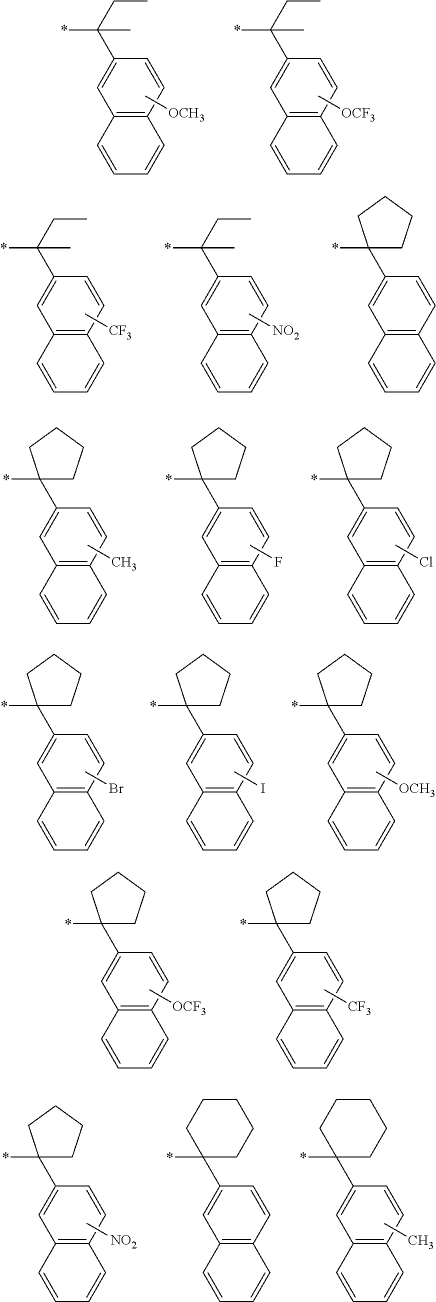

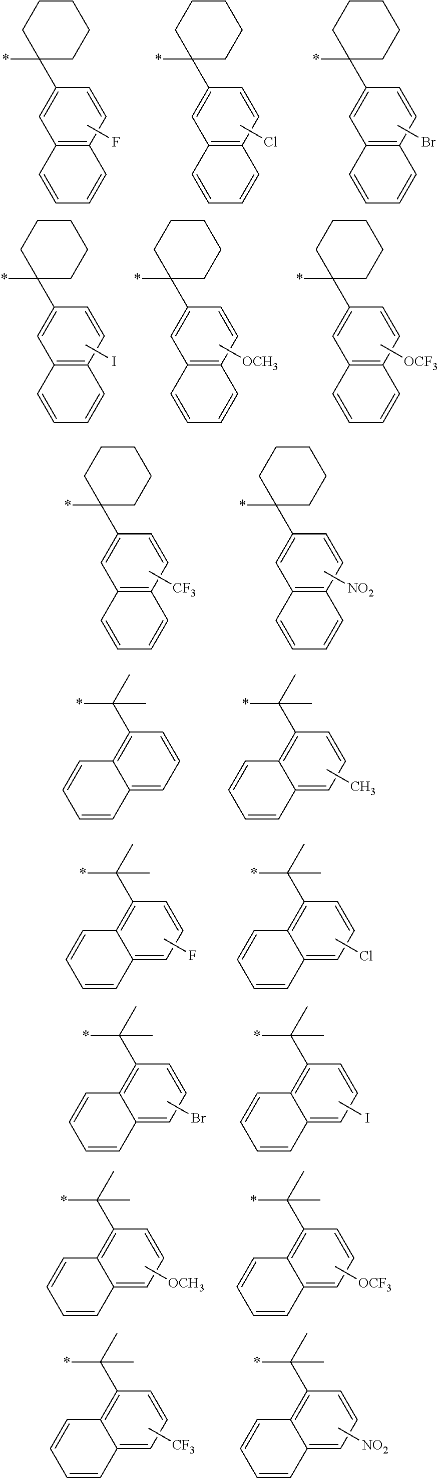

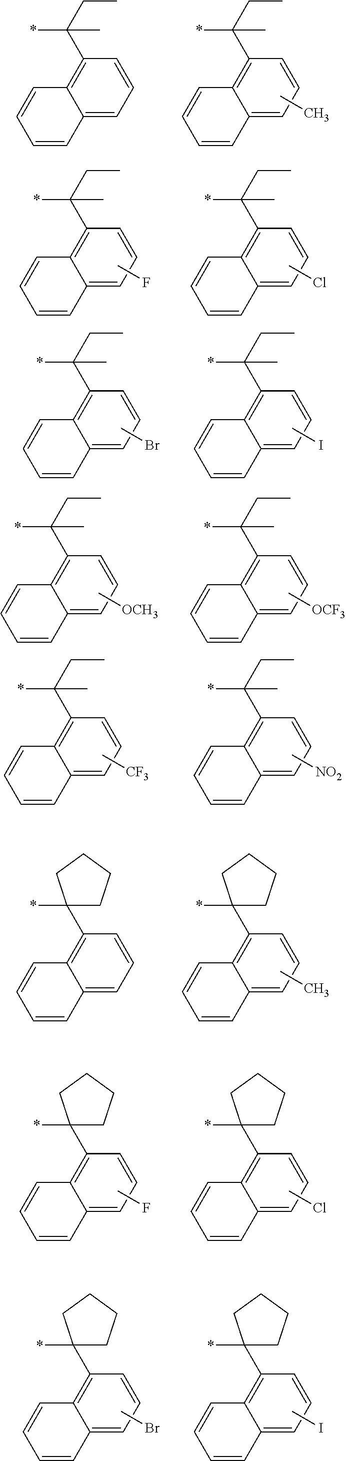

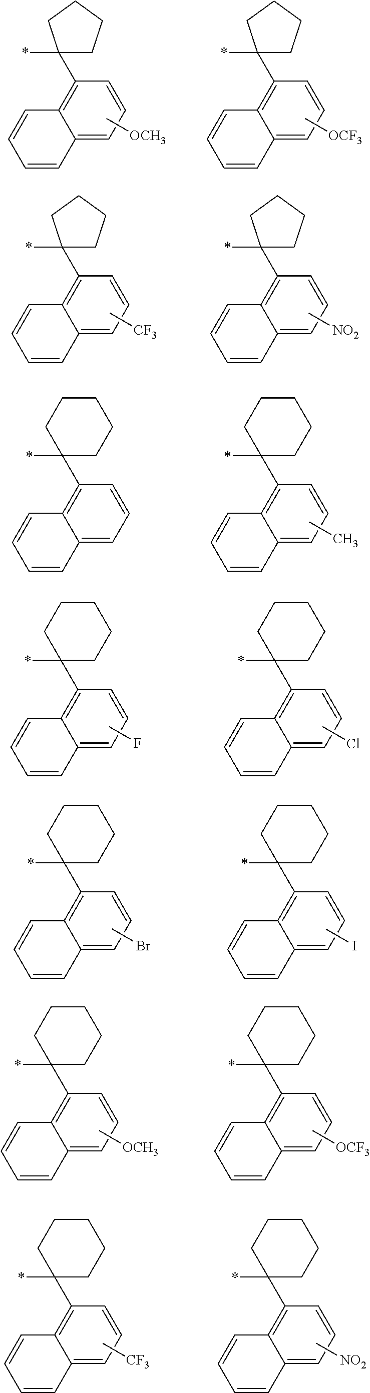

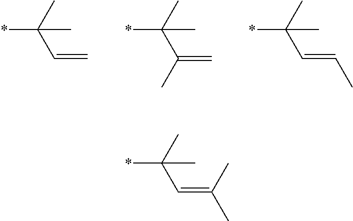

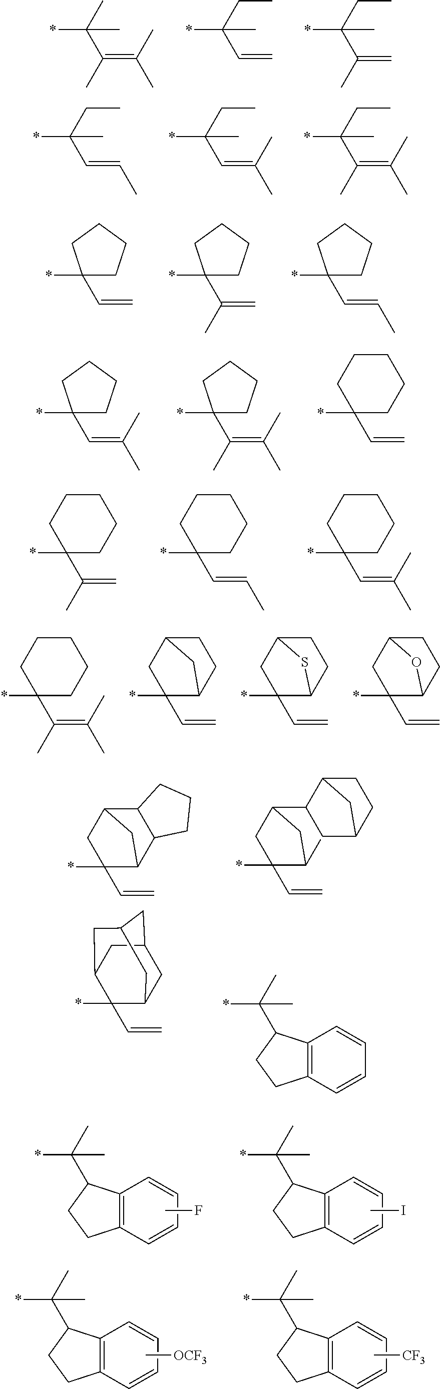

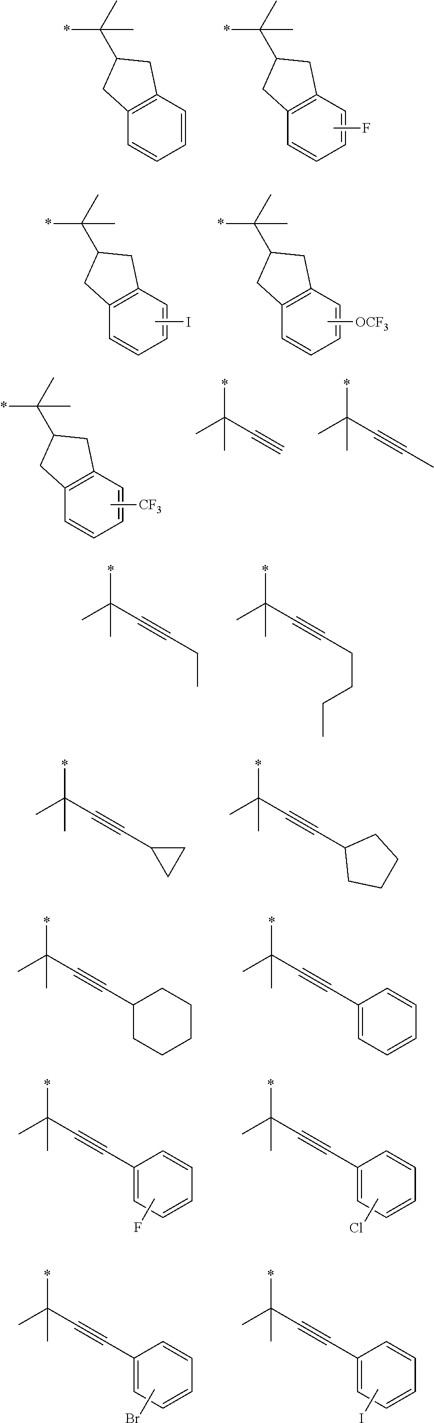

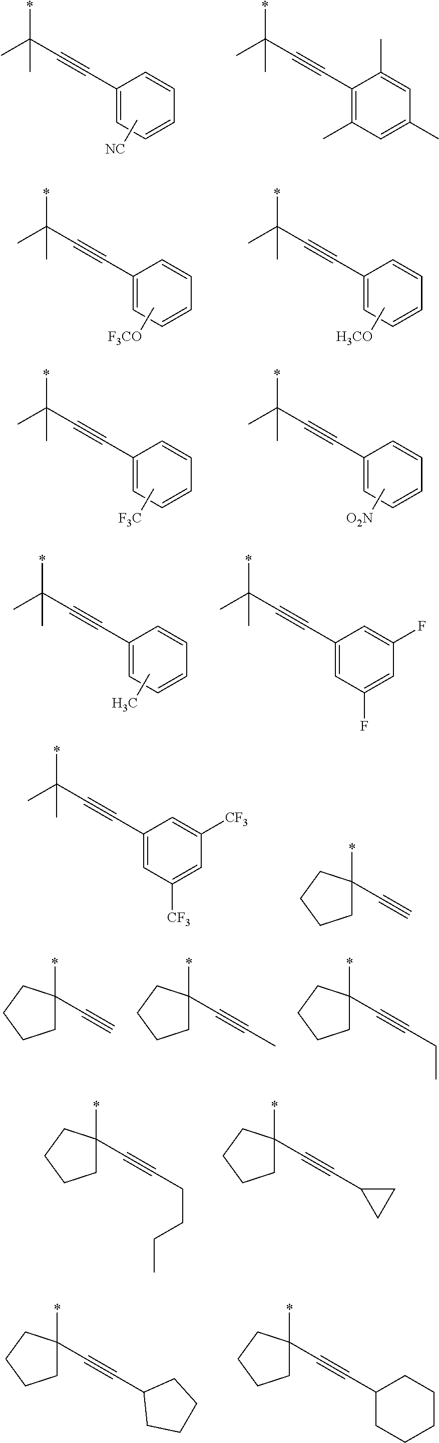

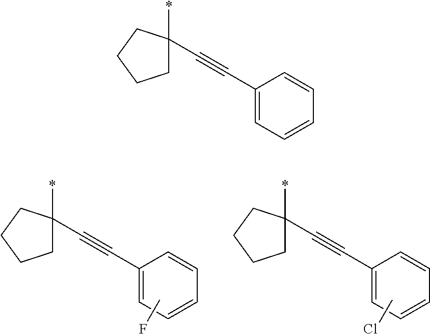

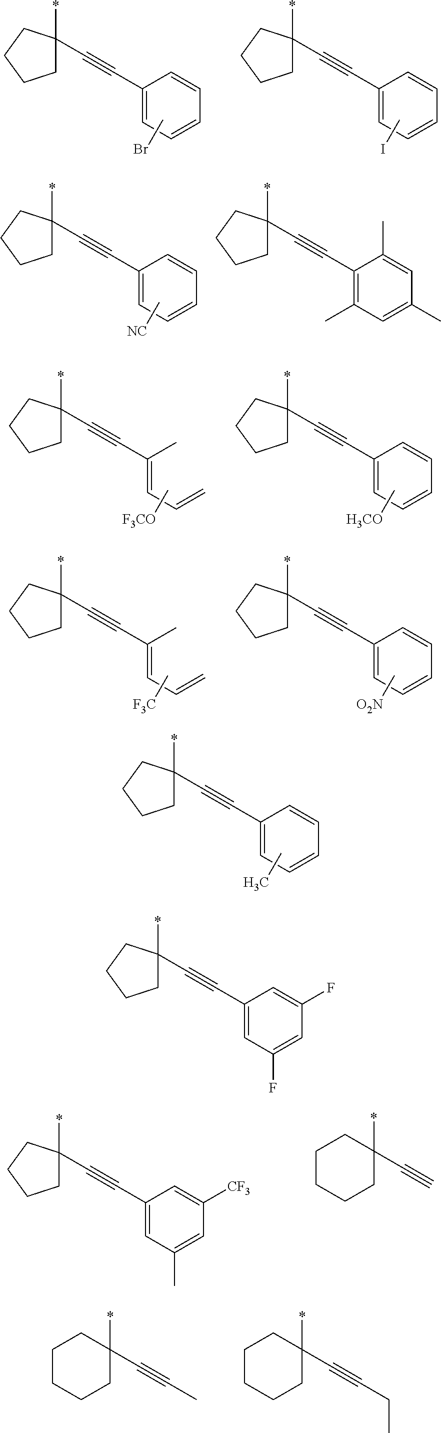

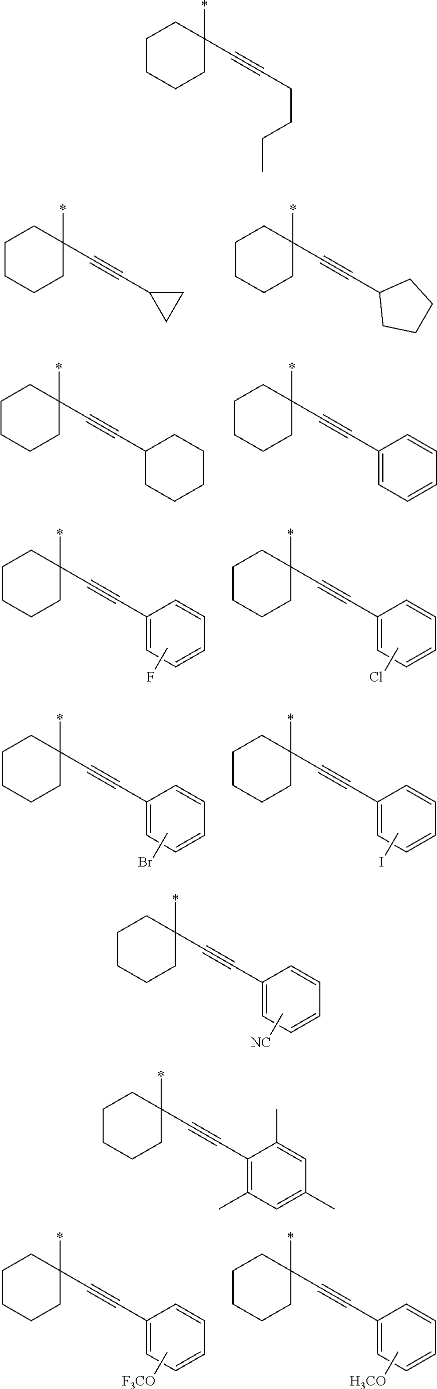

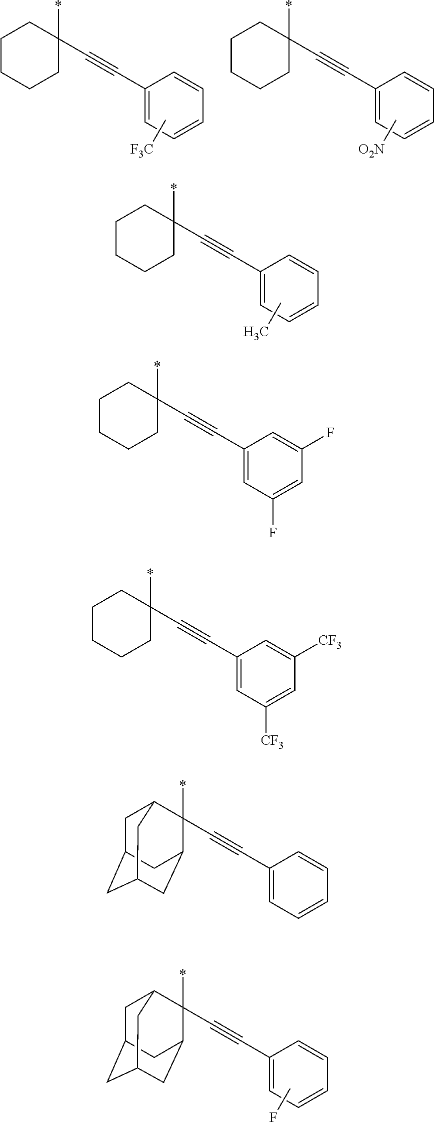

Examples of the acid labile group having the formula (AL-2) include those shown below, but not limited thereto. In the formulae, the asterisk (*) designates a point of attachment to the adjacent oxygen or sulfur.

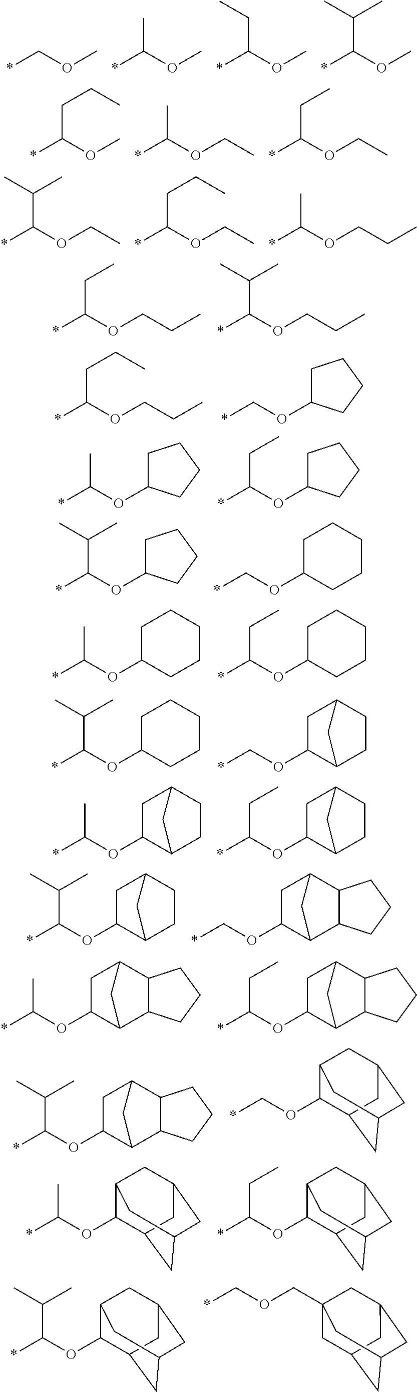

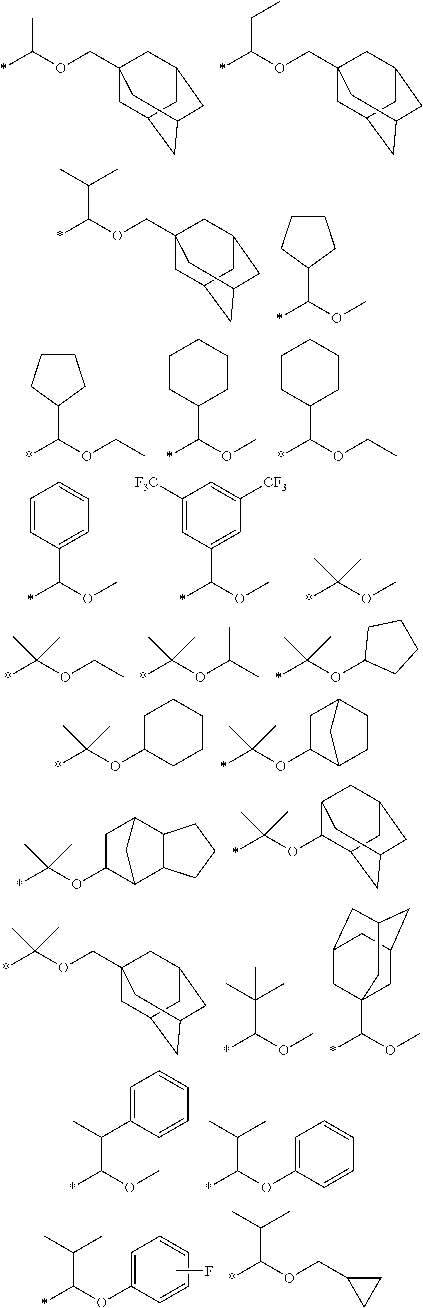

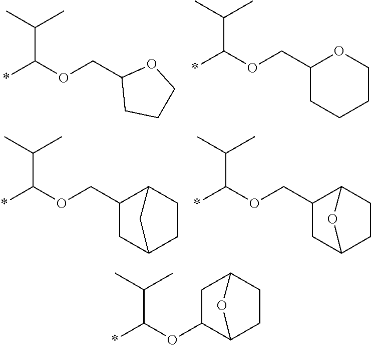

In the formula (1B), R⁴ is halogen exclusive of iodine, nitro, cyano, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Examples of the halogen exclusive of iodine include fluorine, chlorine, and bromine, with fluorine being preferred. The C₁-C₂₀ hydrocarbyl group may be saturated or unsaturated and straight, branched or cyclic. Examples thereof include those exemplified above for the hydrocarbyl group represented by R³. The hydrocarbyl group may contain a heteroatom such as halogen, oxygen or sulfur.

When n14 is 2, 3 or 4, a plurality of R⁴ may bond together to form a ring structure with the carbon atoms on the aromatic ring to which they are attached, some —CH₂— in the ring may be replaced by —O— or —S—. Exemplary rings include cyclopropane, cyclobutane, cyclopentane, cyclohexane, cycloheptane, cyclooctane, norbornane, adamantane, tricyclo[5.2.1.0^{2,6}]decane, and tetracyclo[6.2.1.1^{3,6}.0^{2,7}]dodecane rings as well as these rings in which some —CH₂— may be replaced by —O— or —S—.

As the carboxylic acid anion having the formula (1B), a carboxylic acid anion having the formula (1B-1) is preferred.

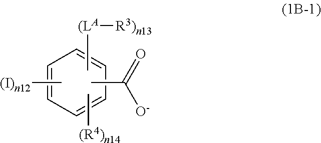

wherein n12 to n14, L^{A}, R³ and R⁴ are as defined above.

Examples of the anion in the onium salt having the formula (1B) include those shown below, but not limited thereto.

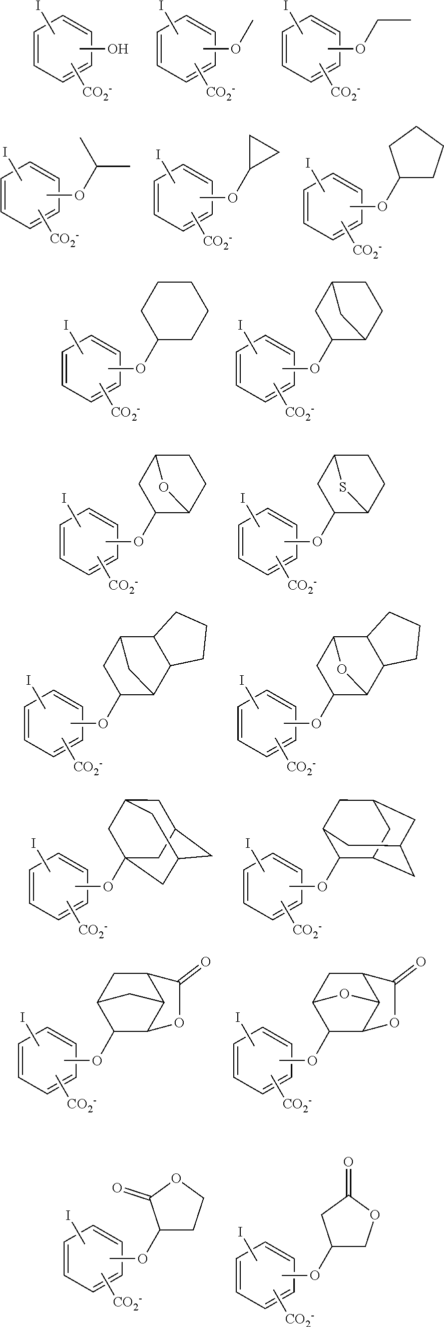

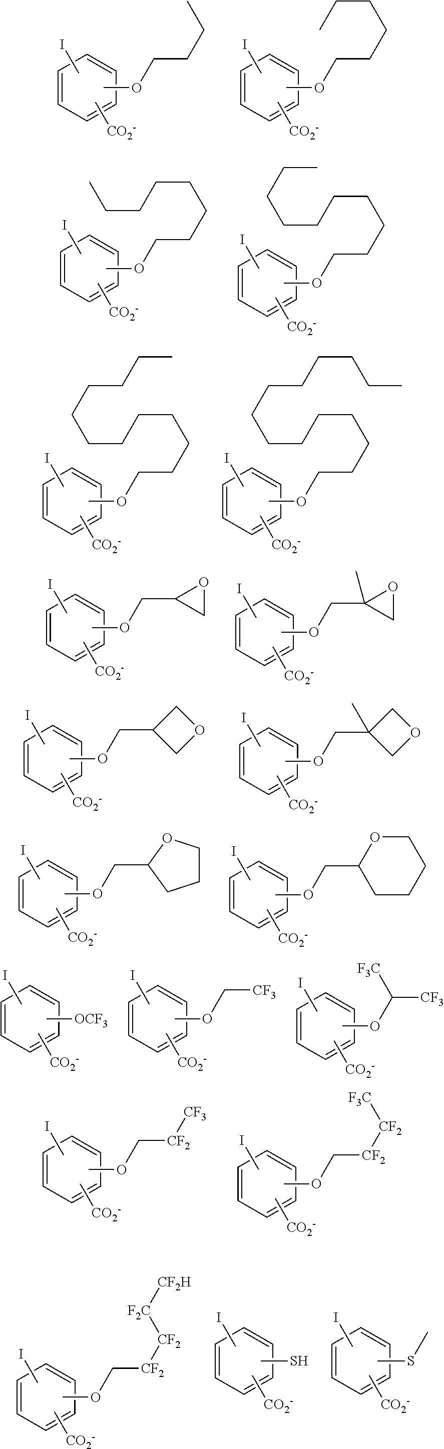

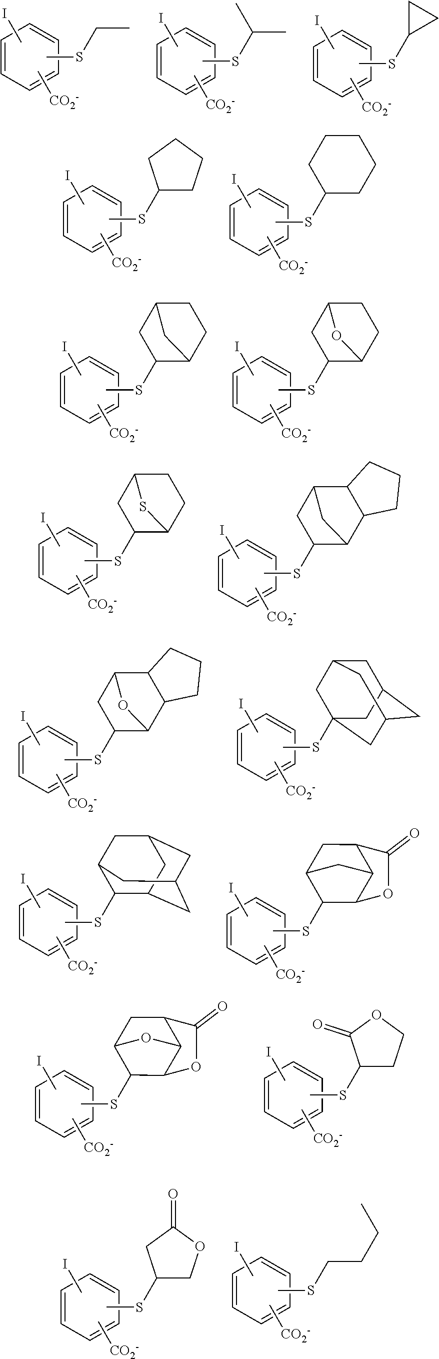

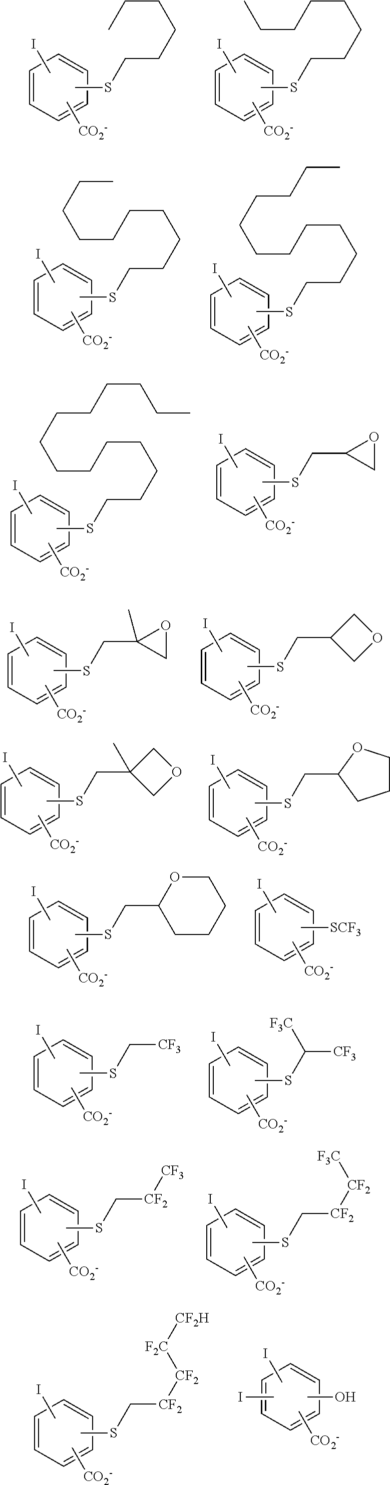

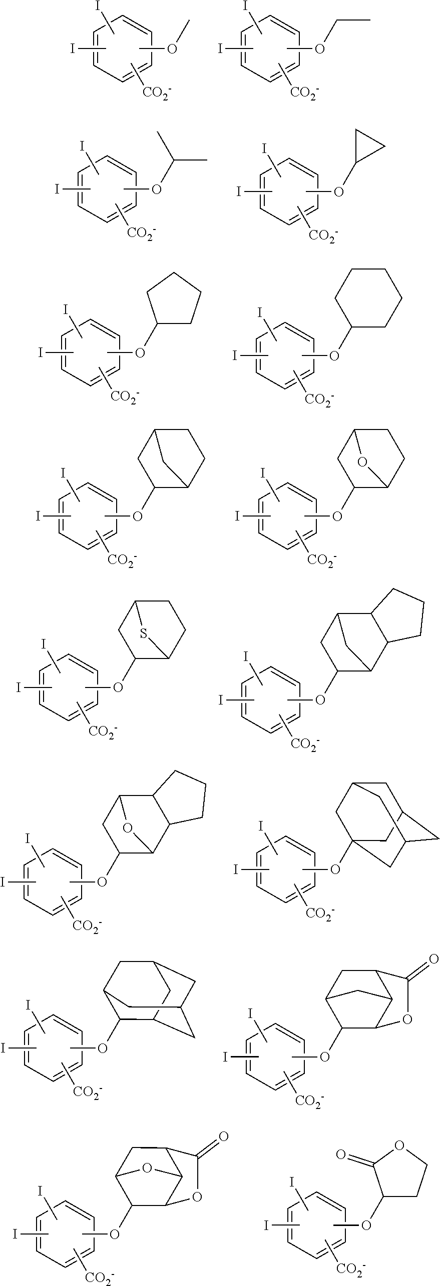

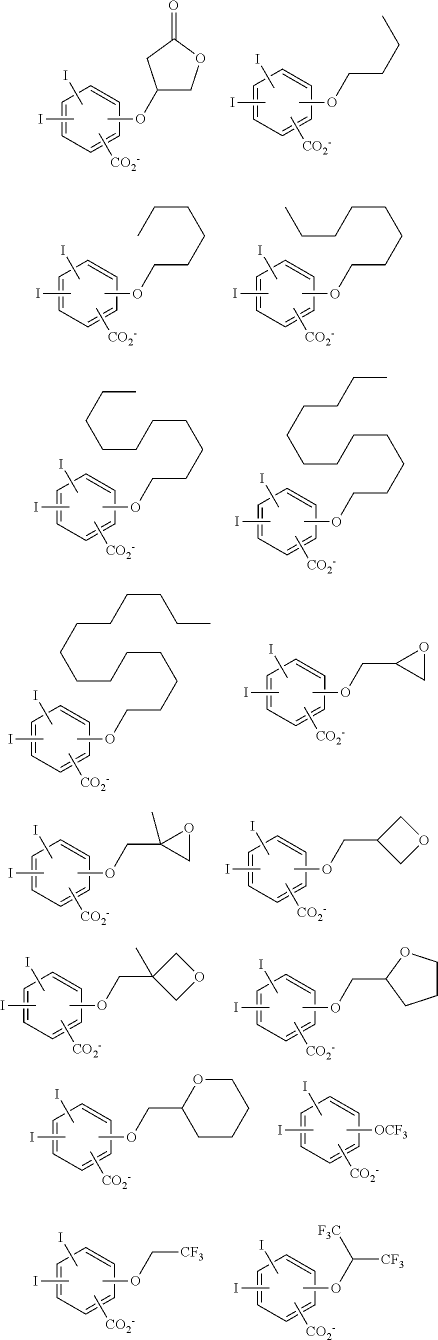

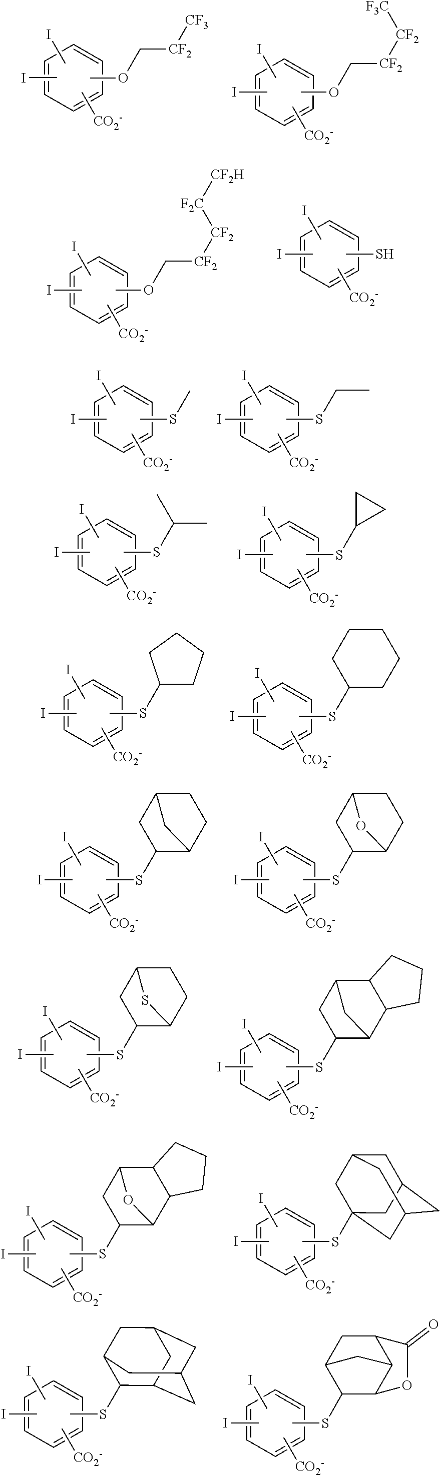

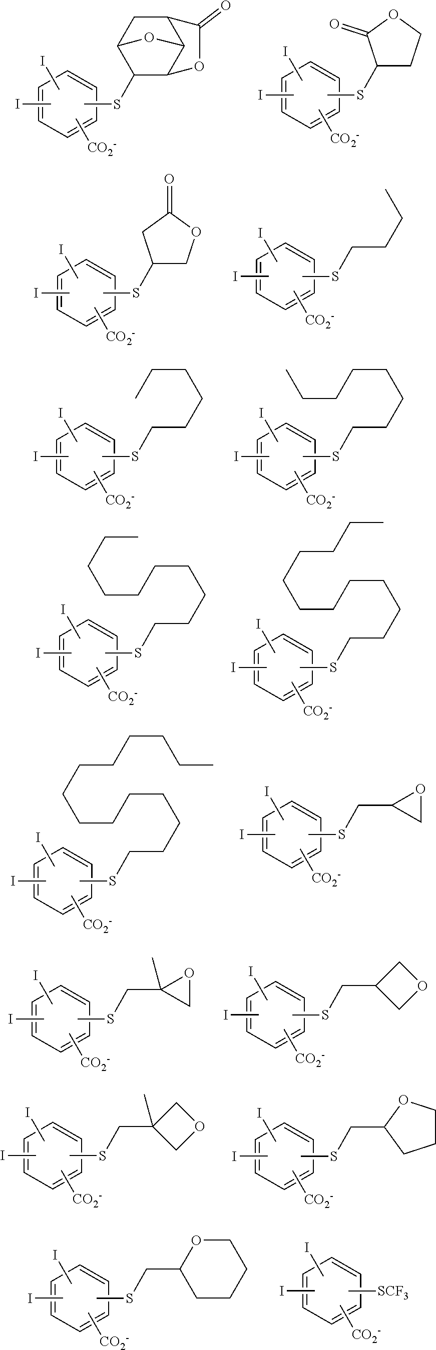

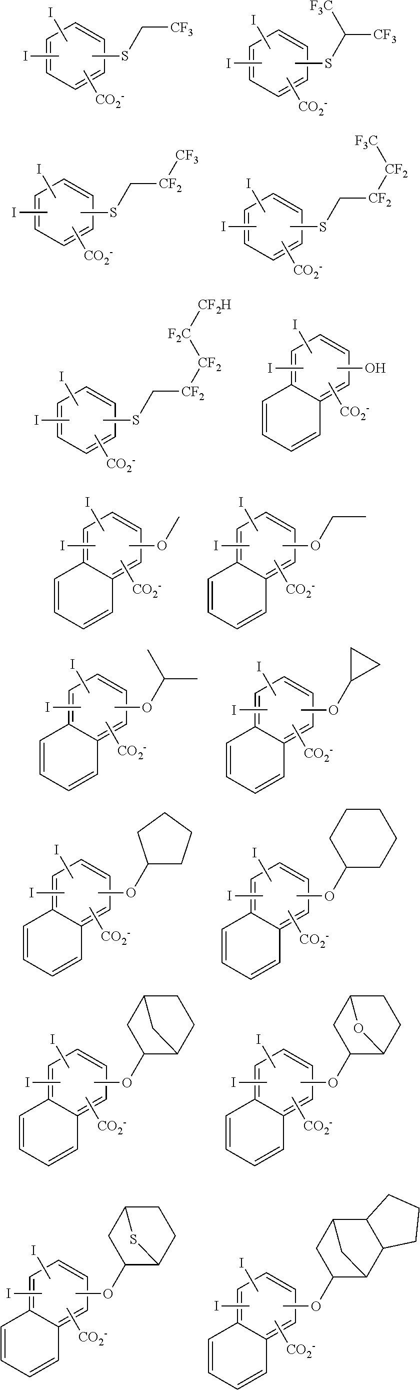

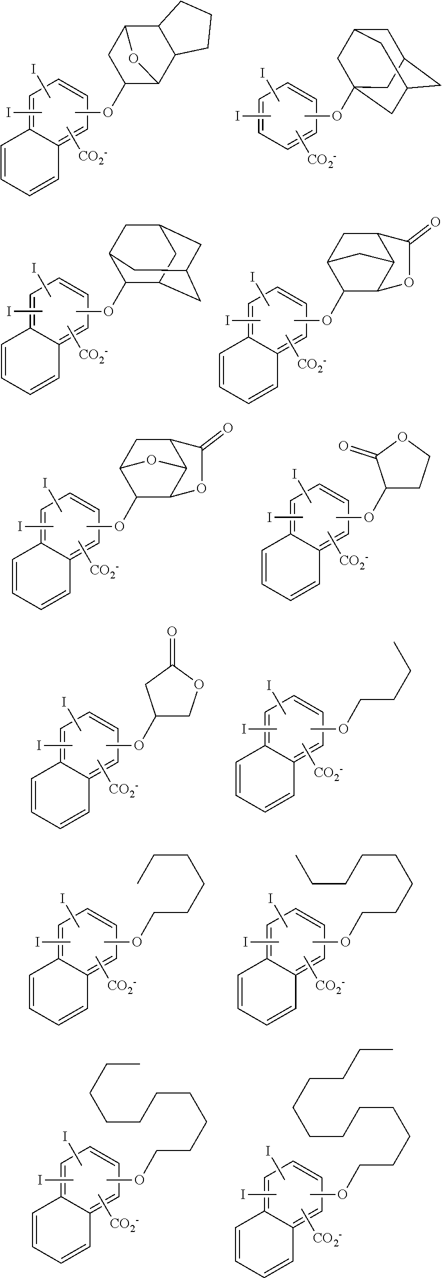

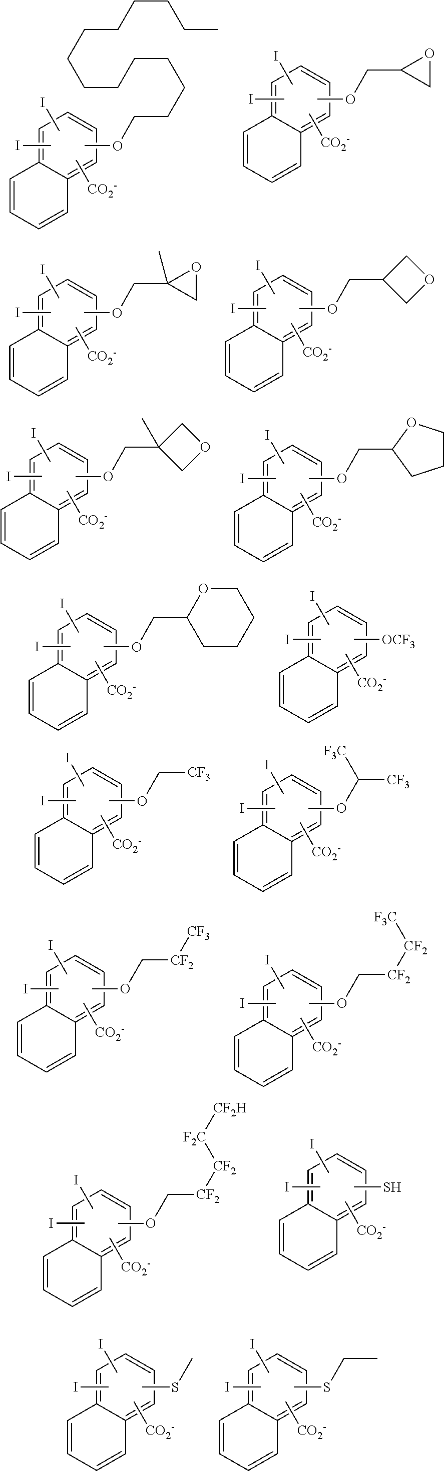

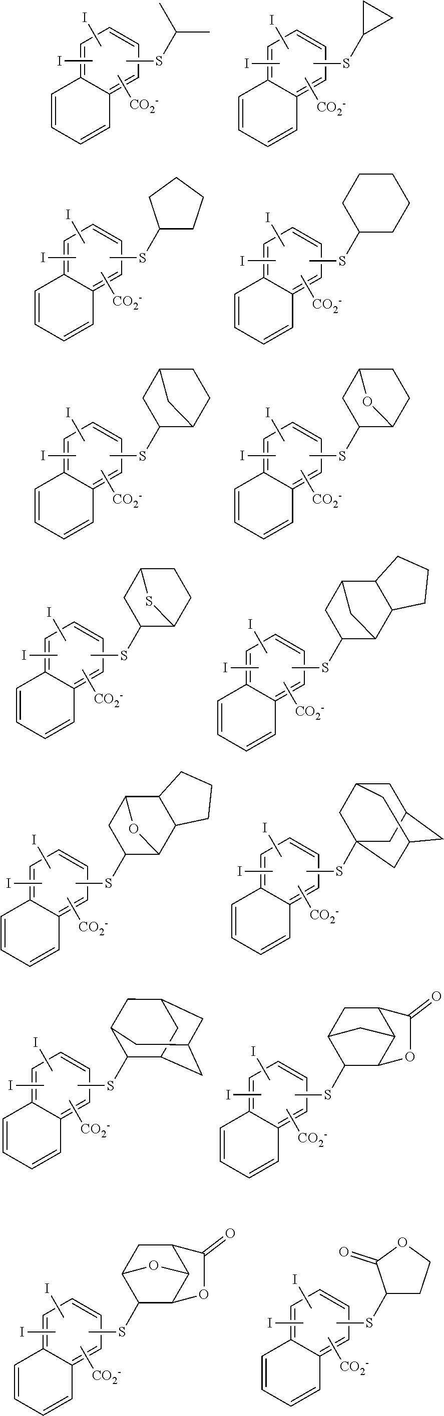

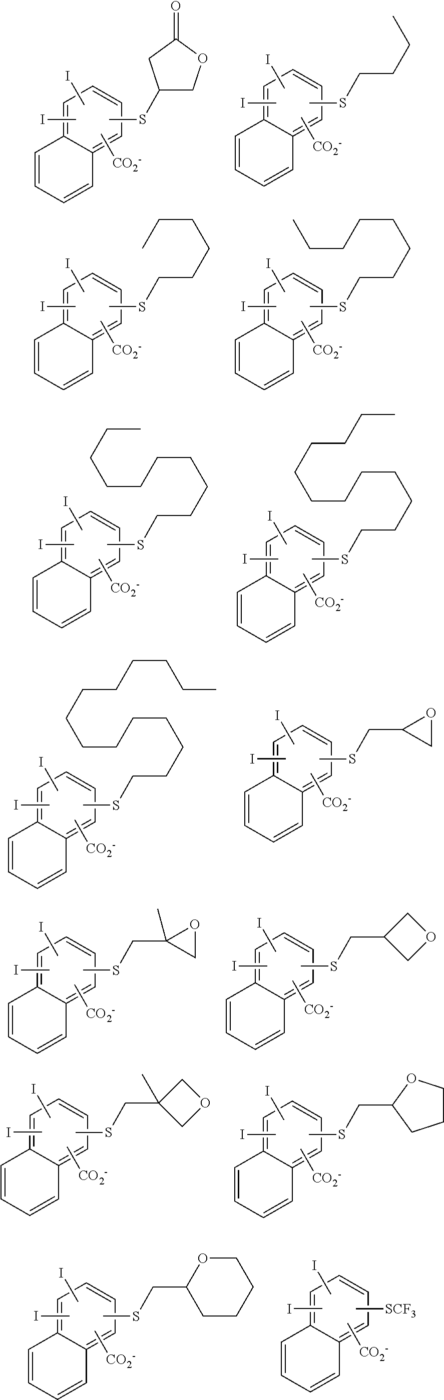

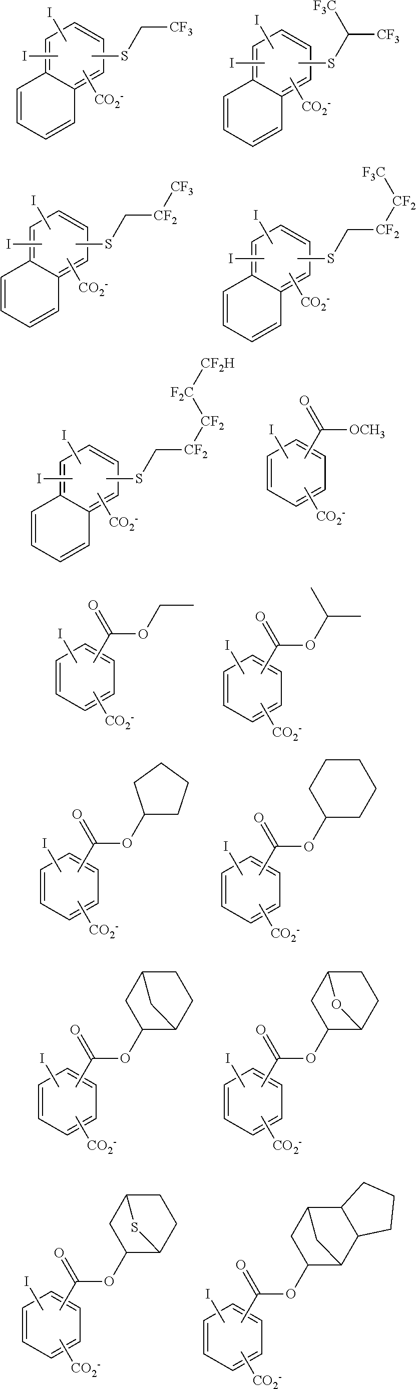

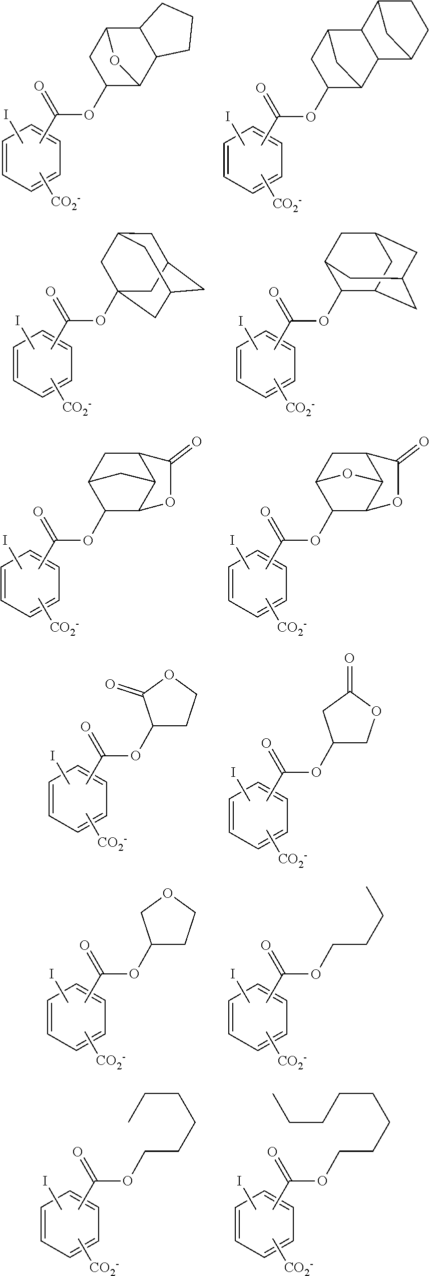

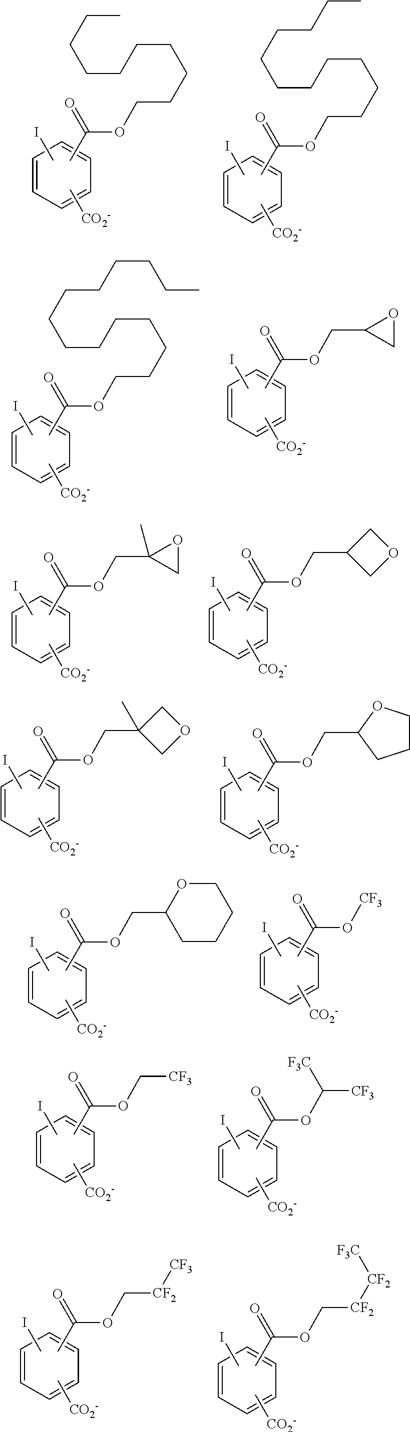

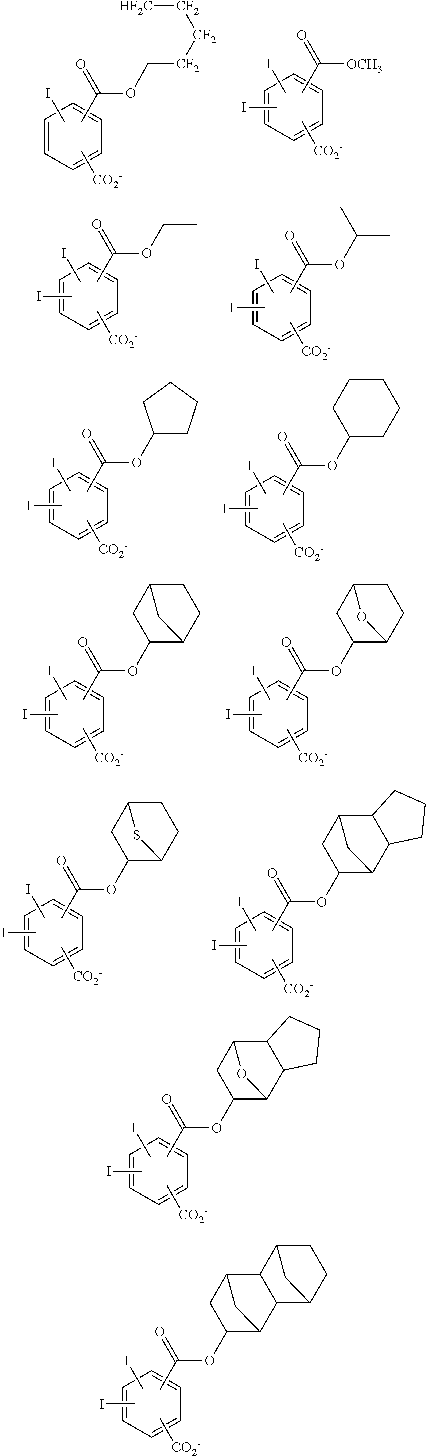

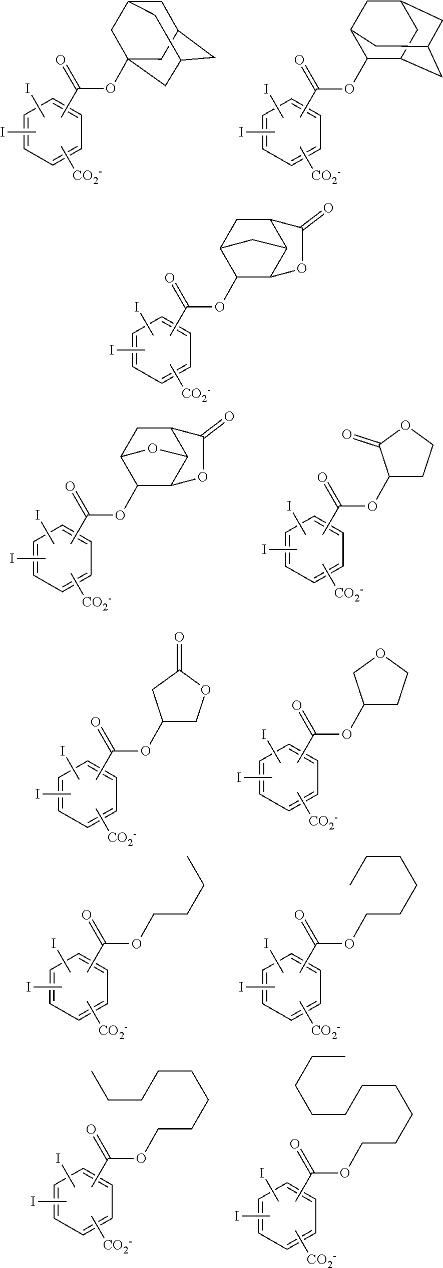

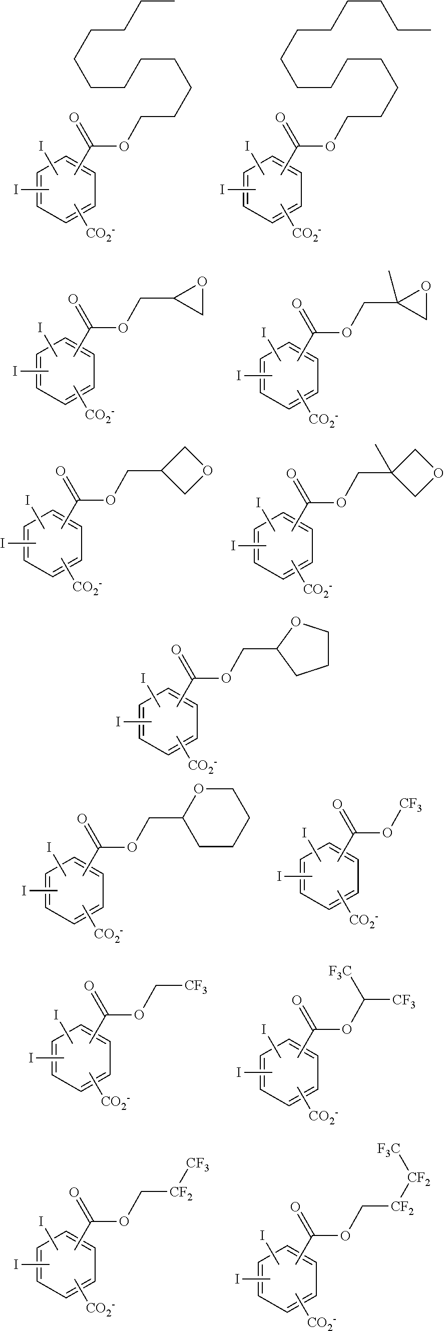

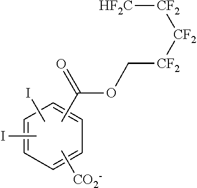

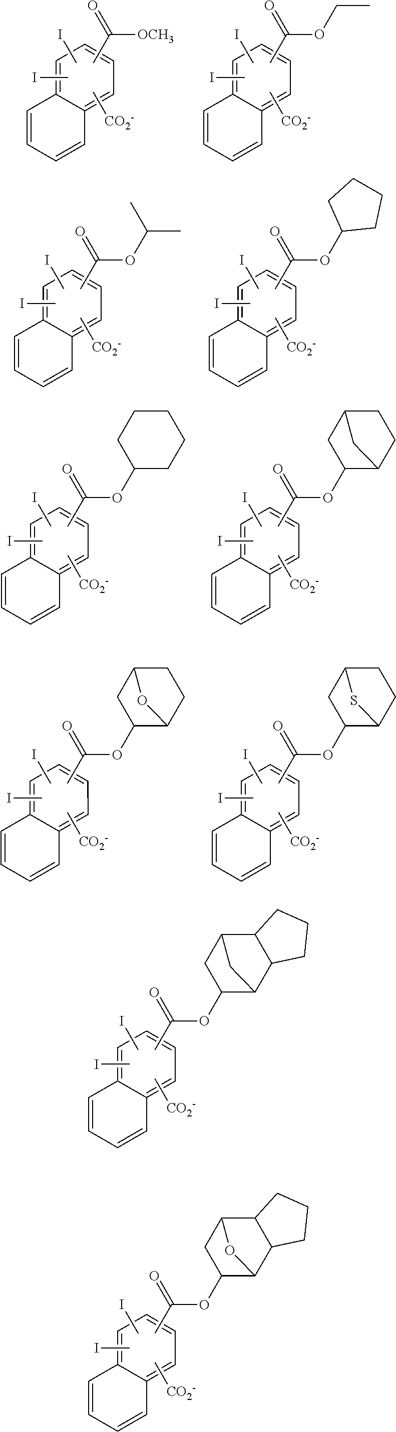

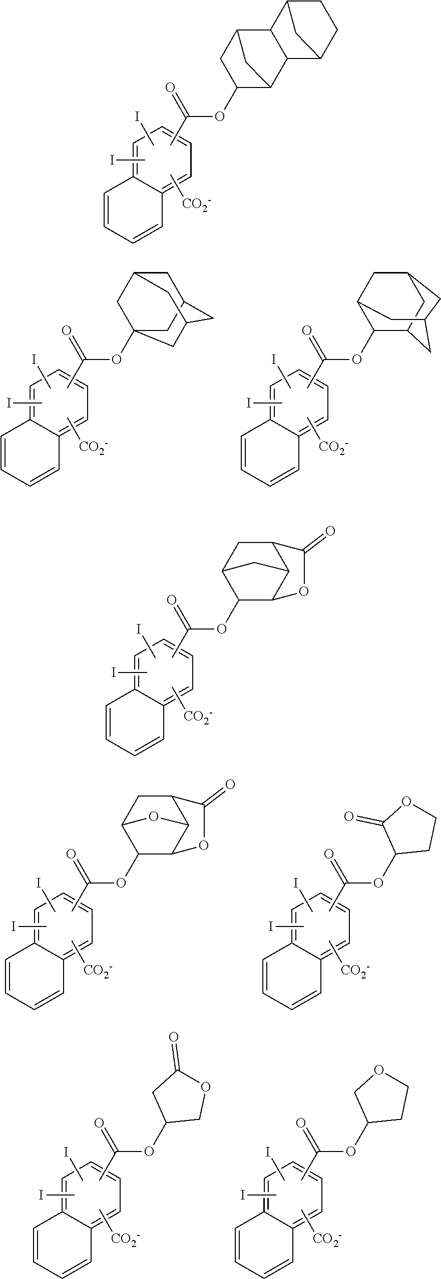

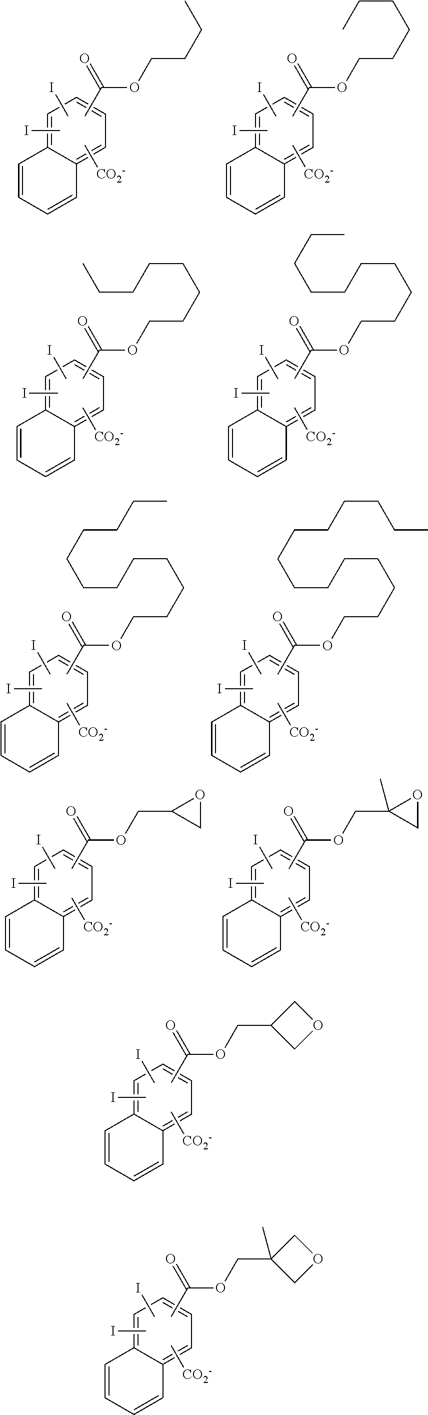

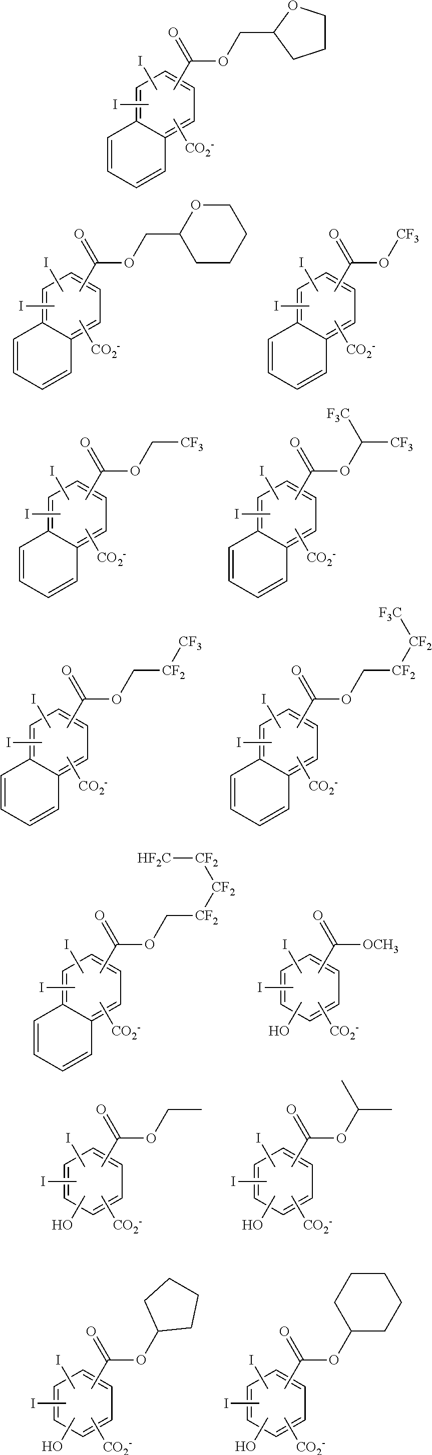

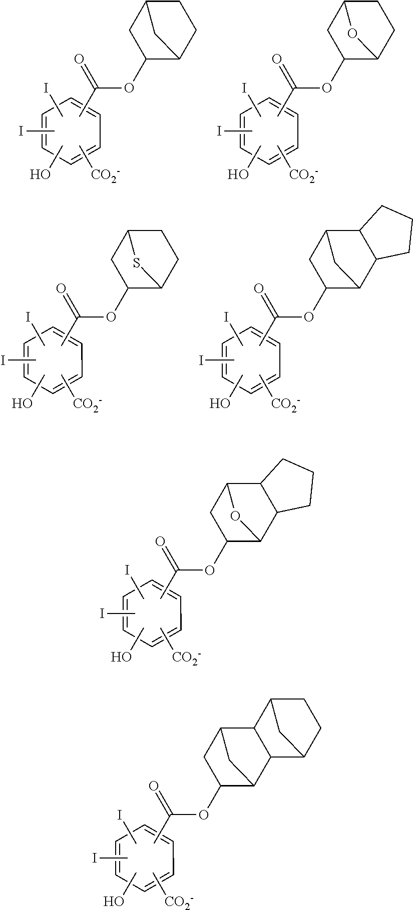

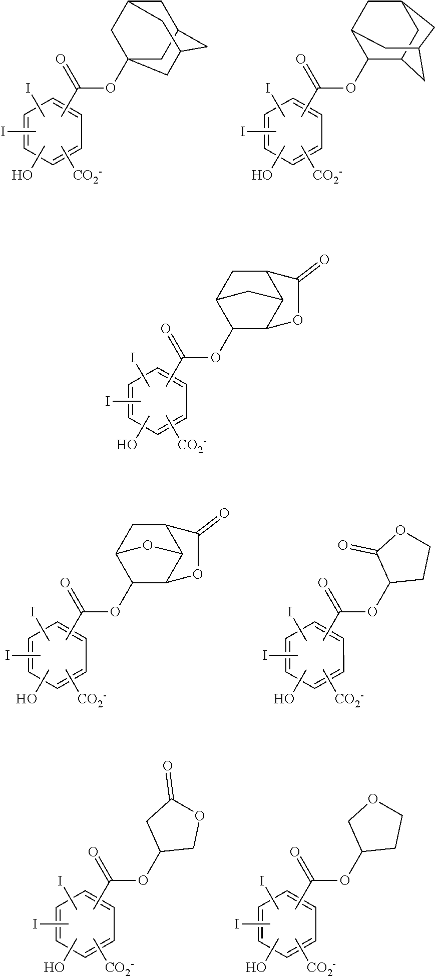

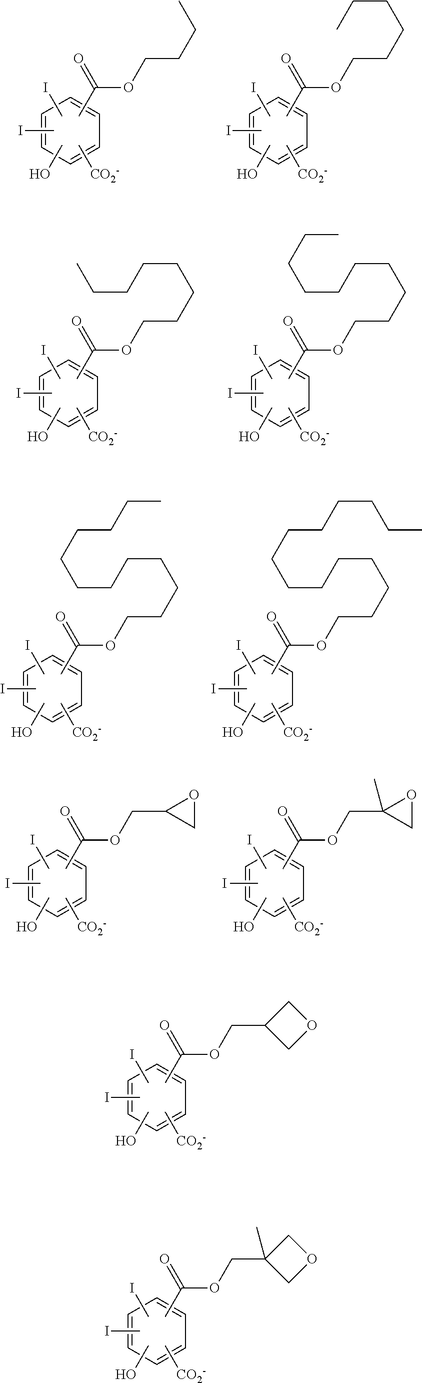

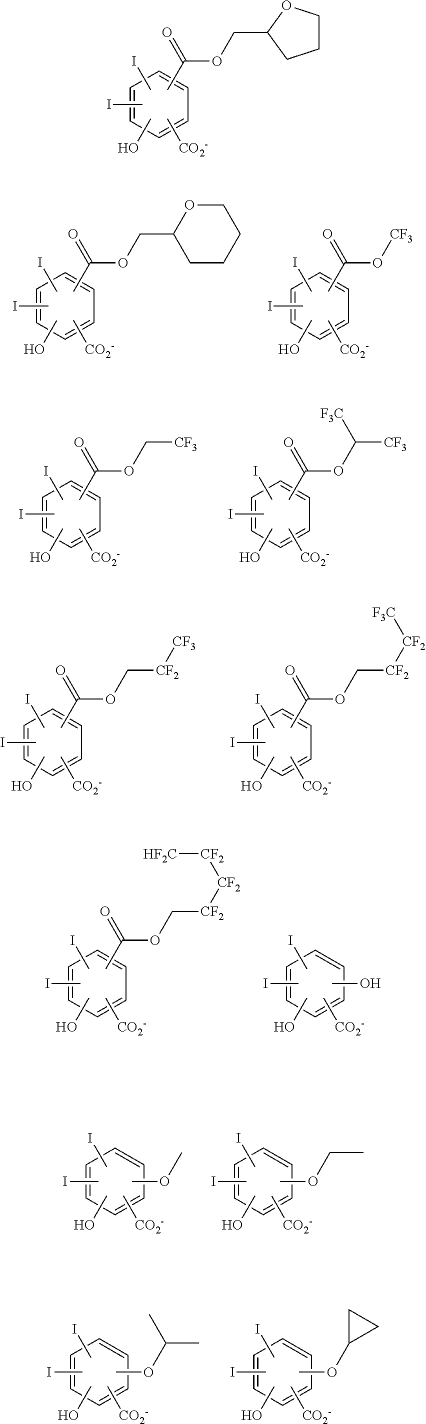

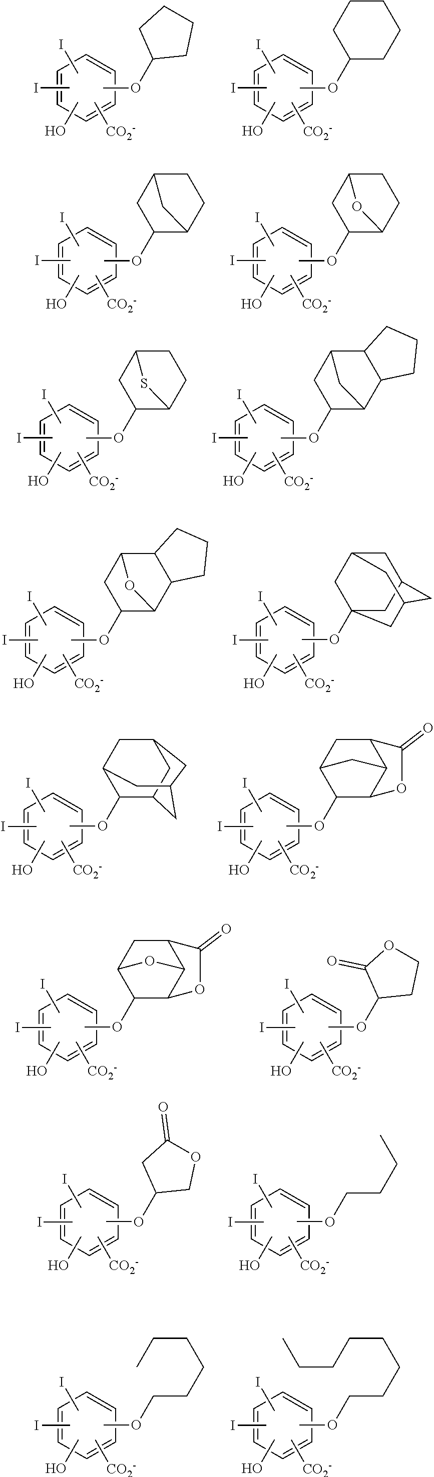

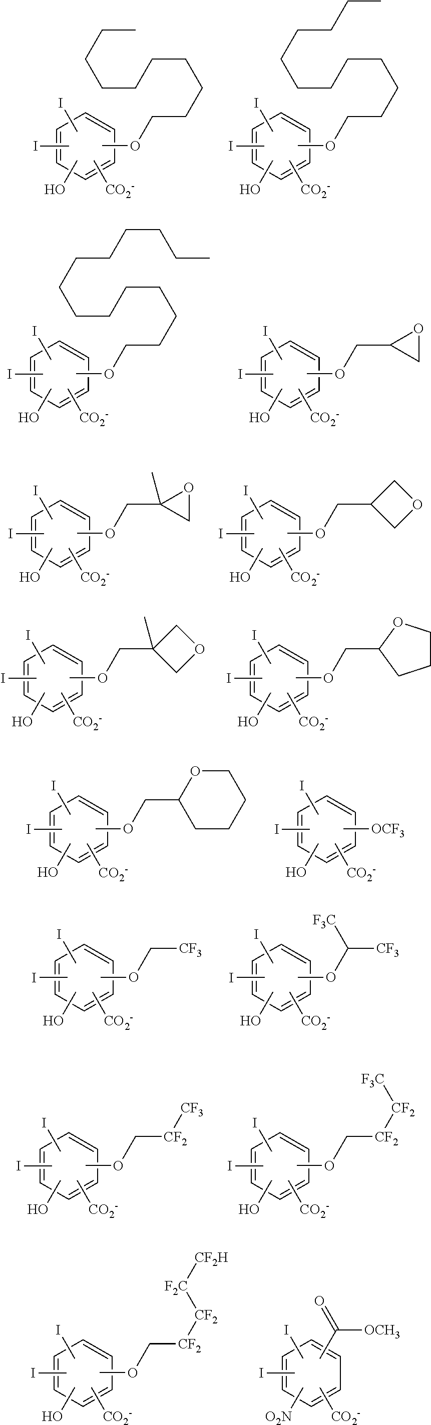

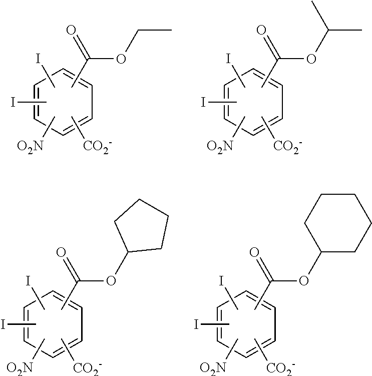

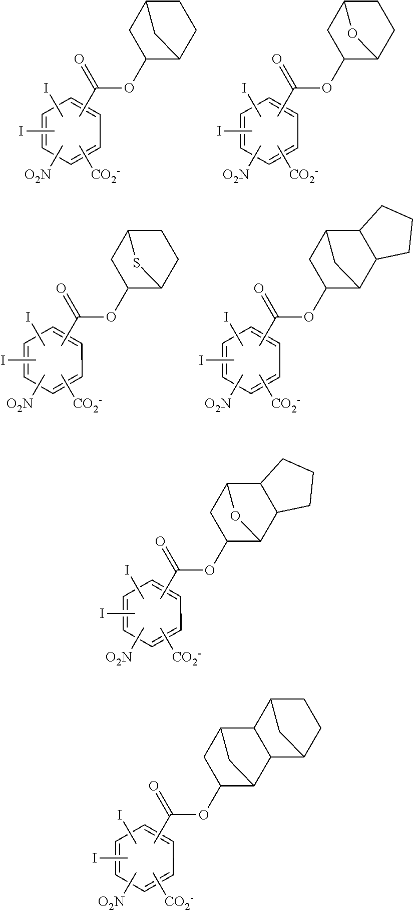

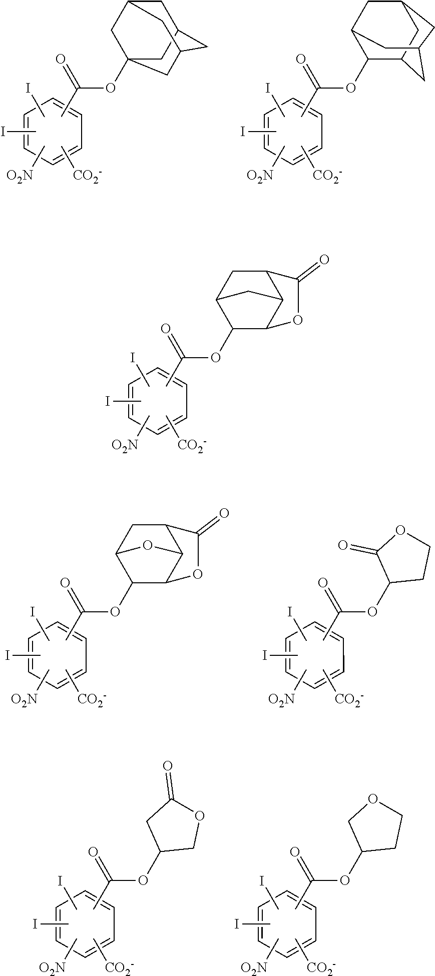

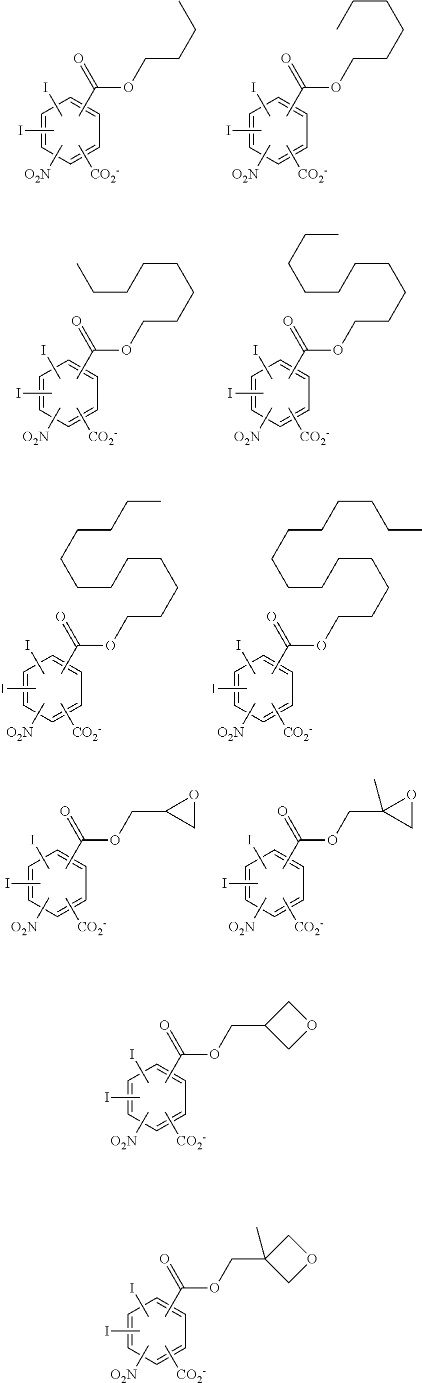

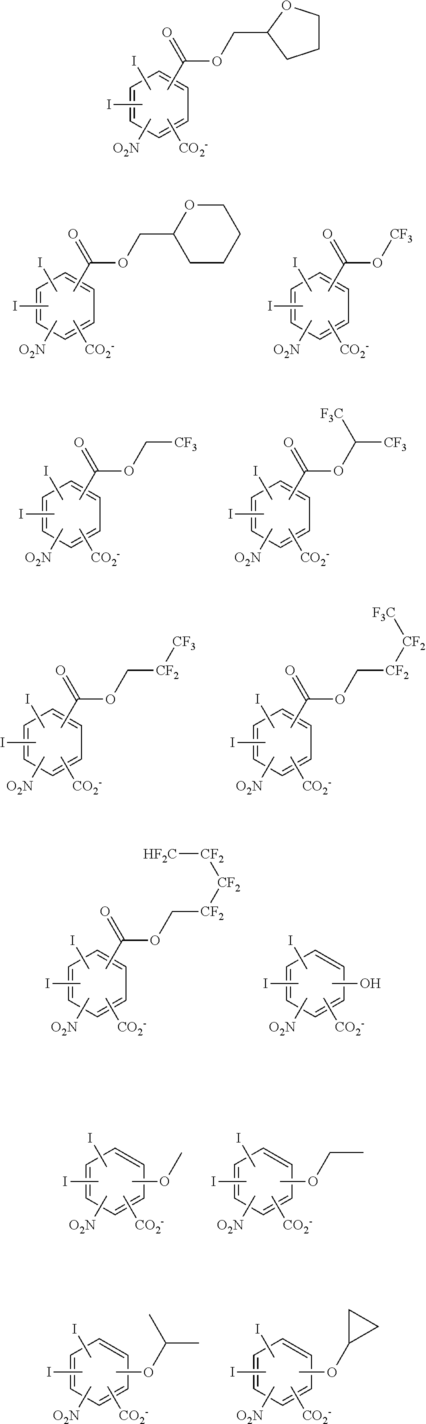

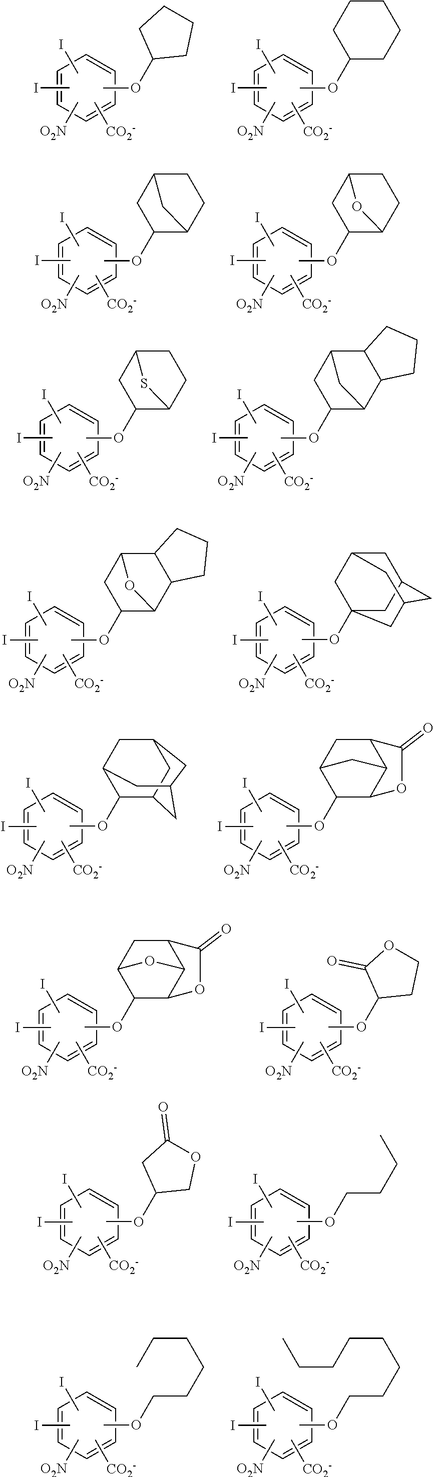

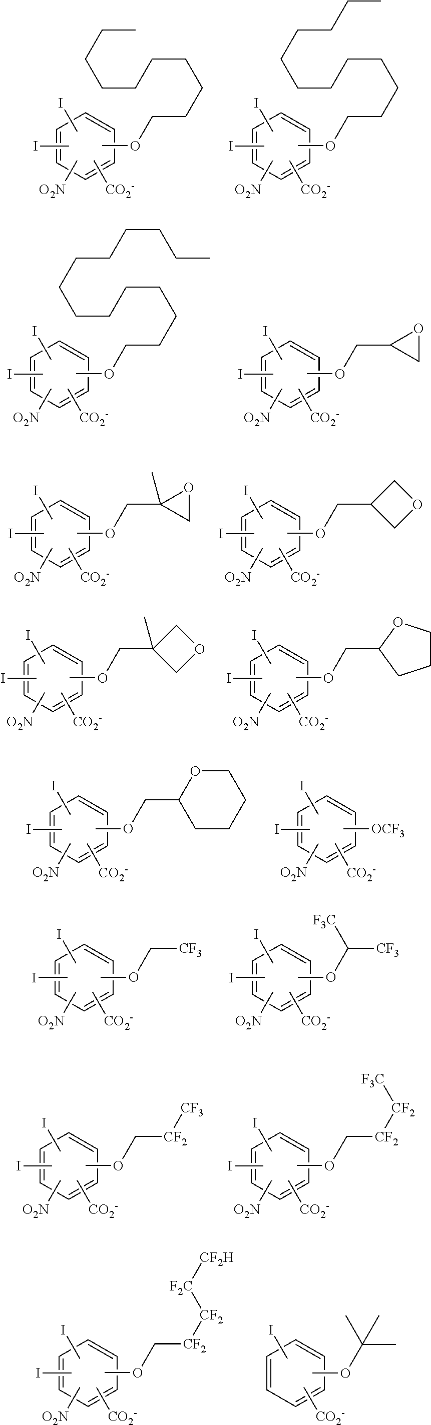

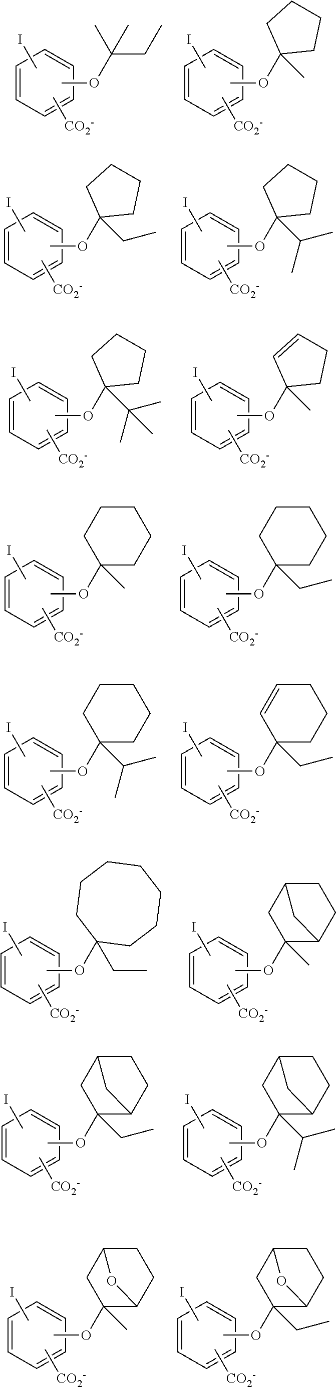

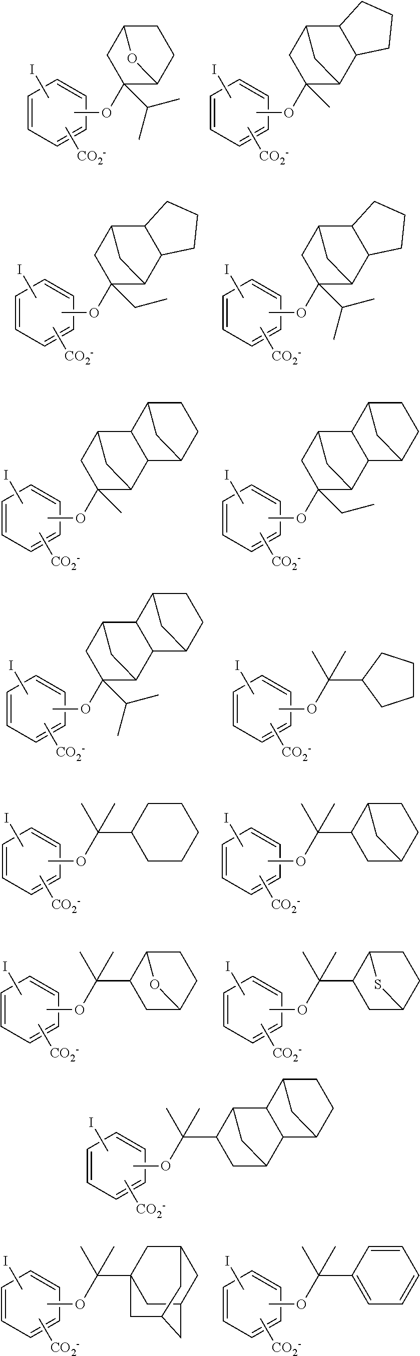

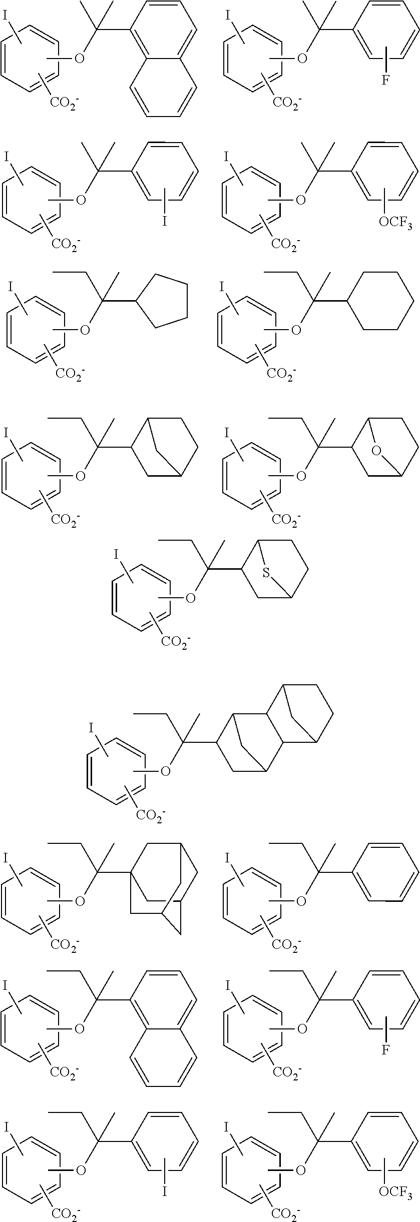

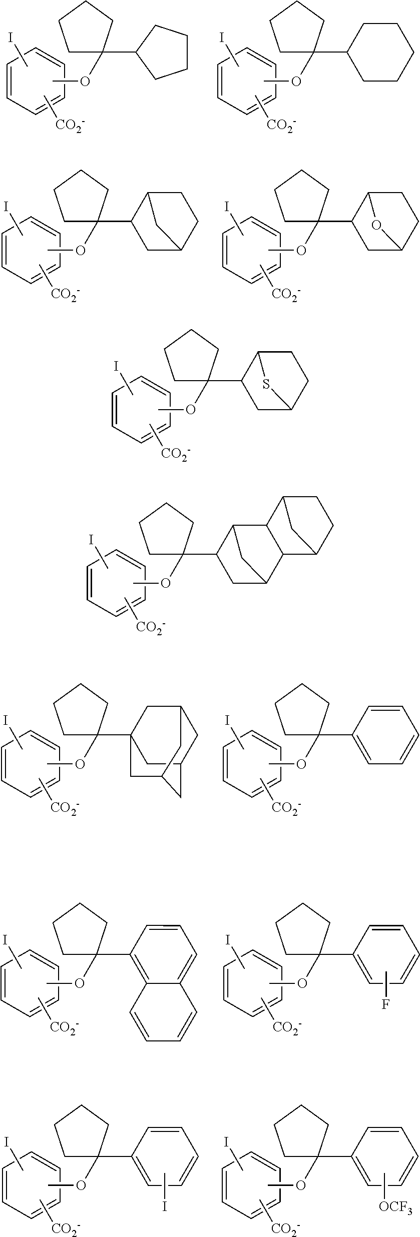

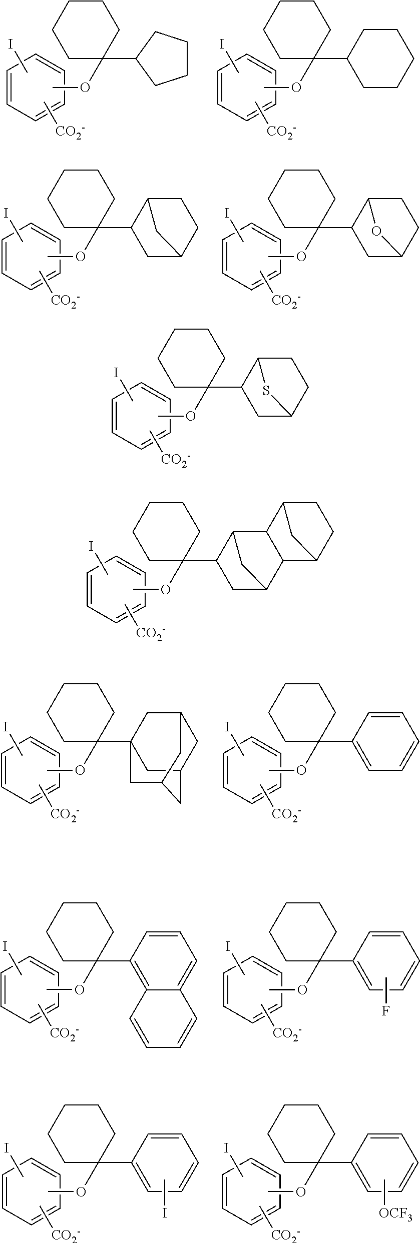

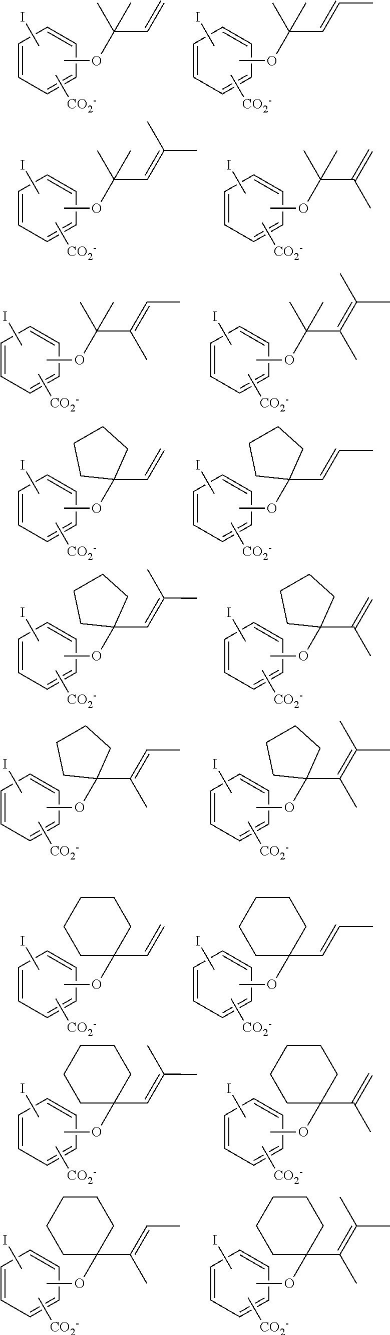

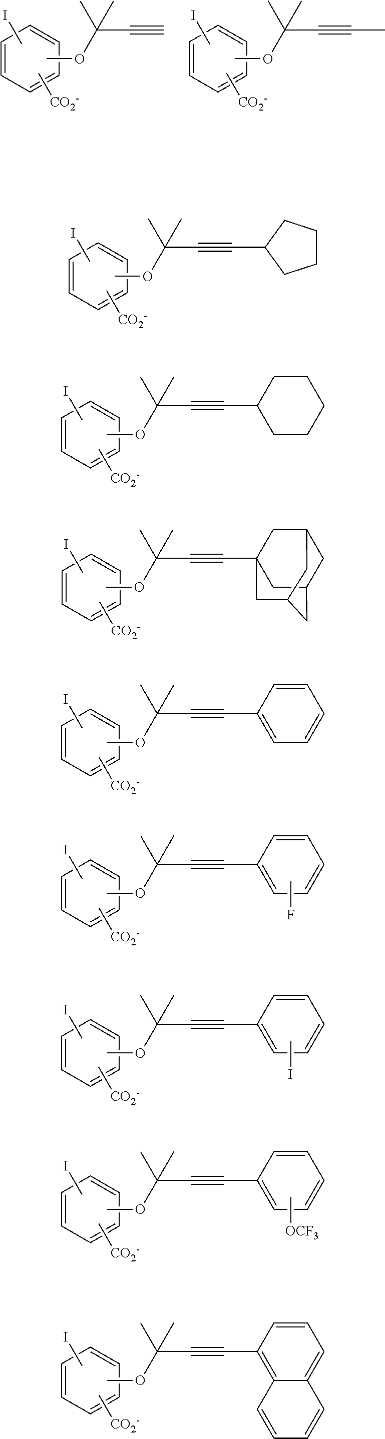

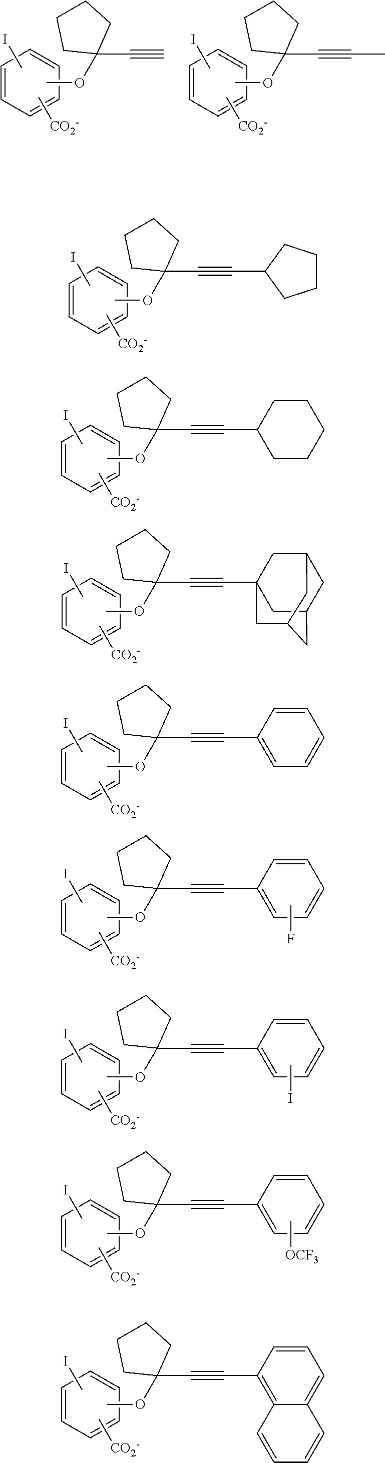

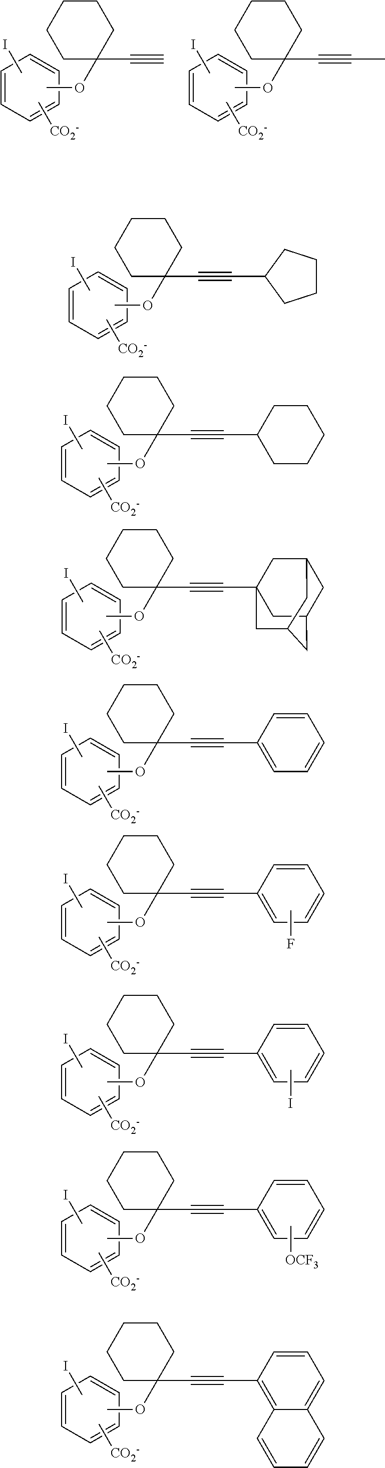

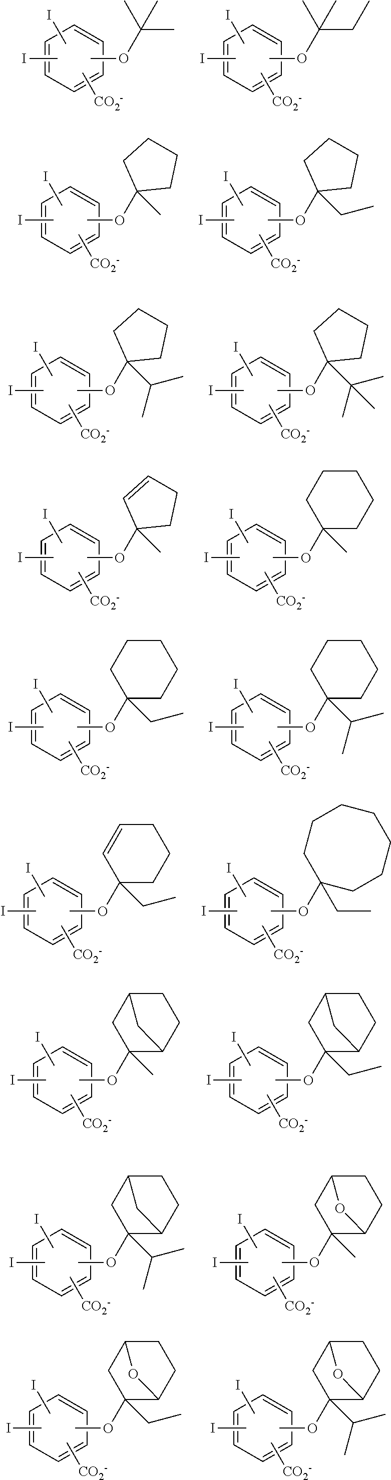

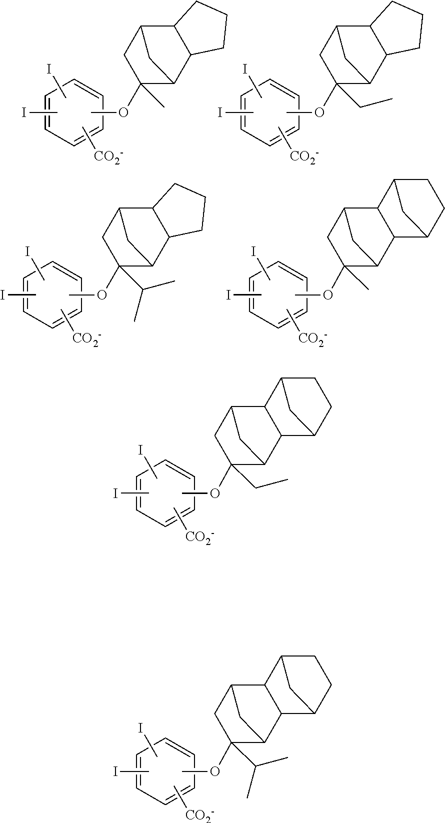

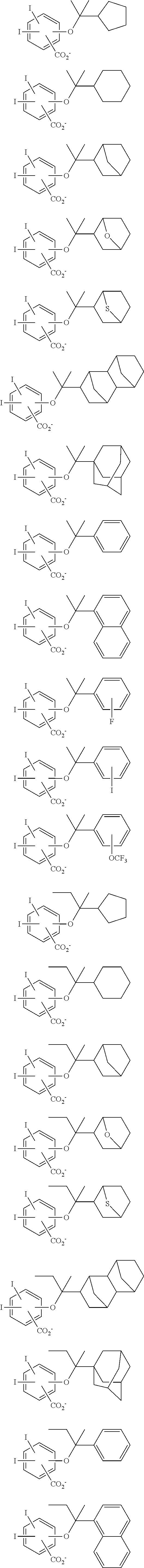

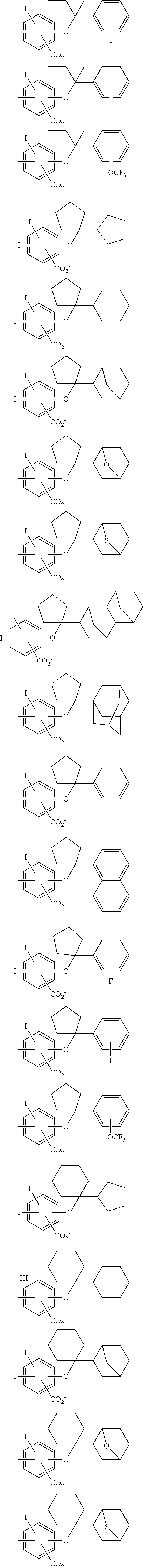

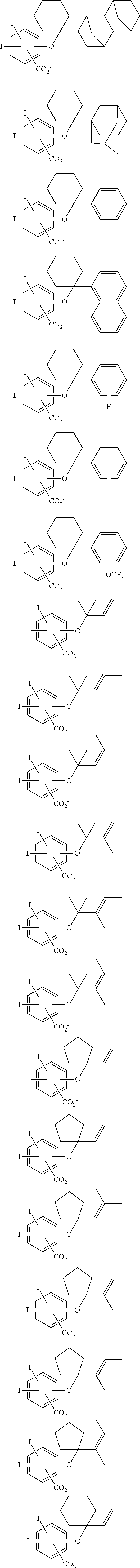

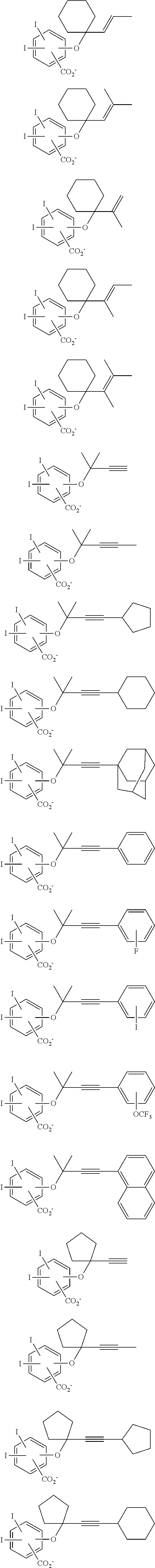

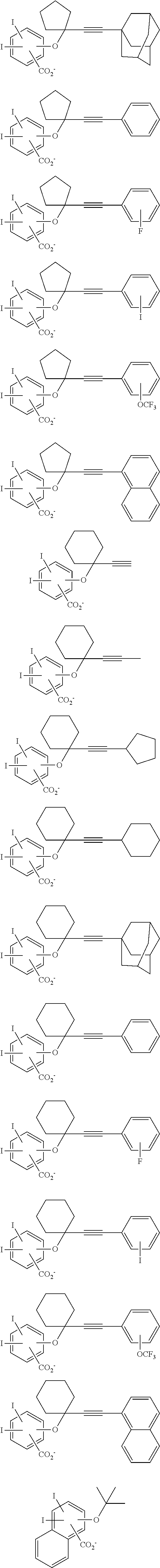

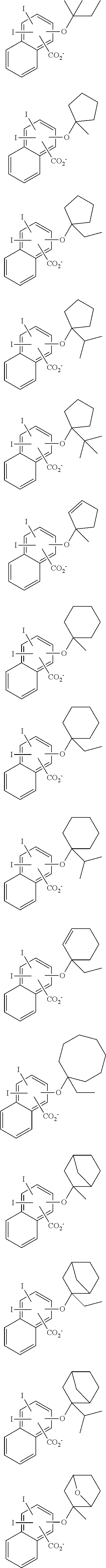

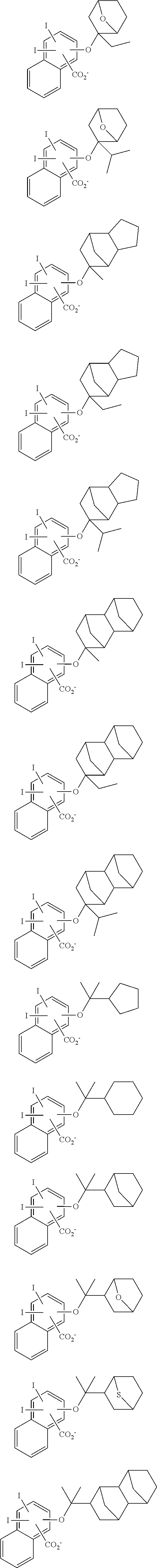

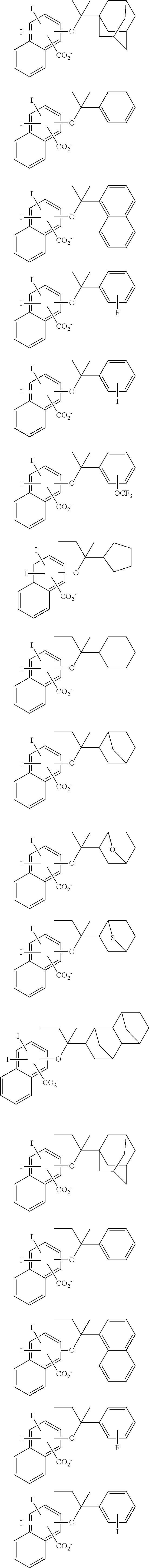

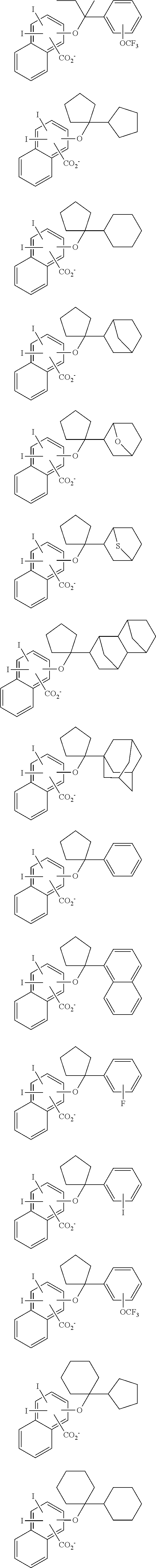

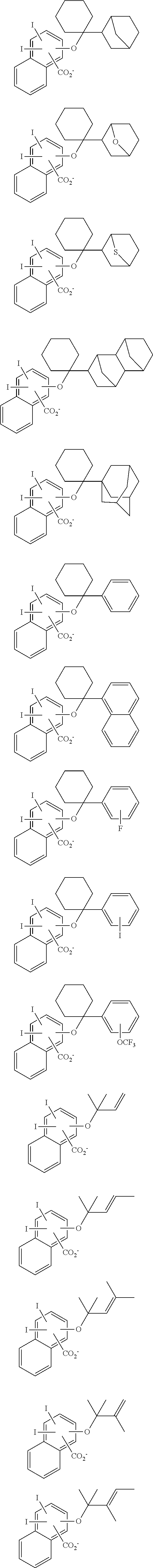

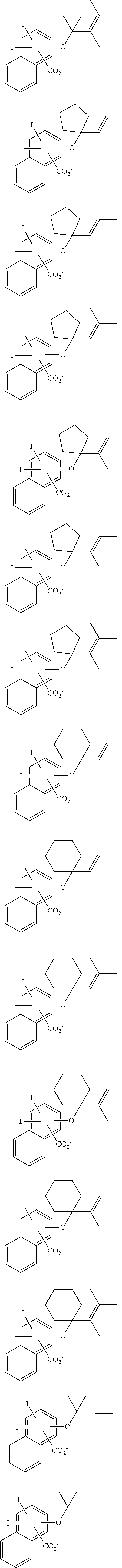

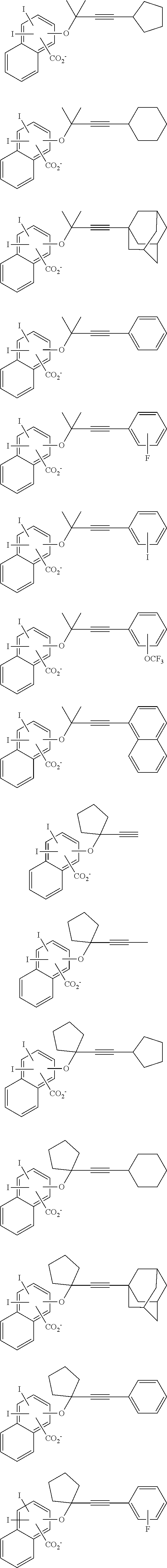

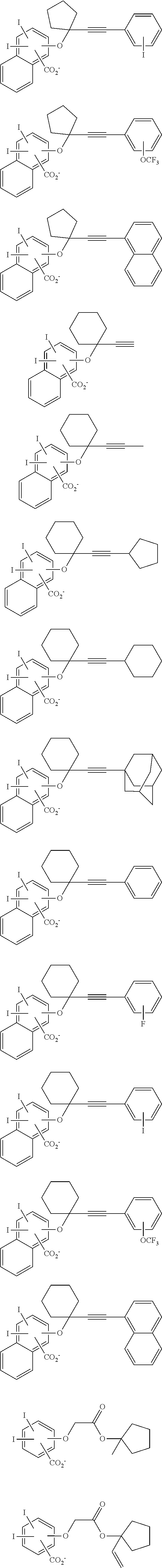

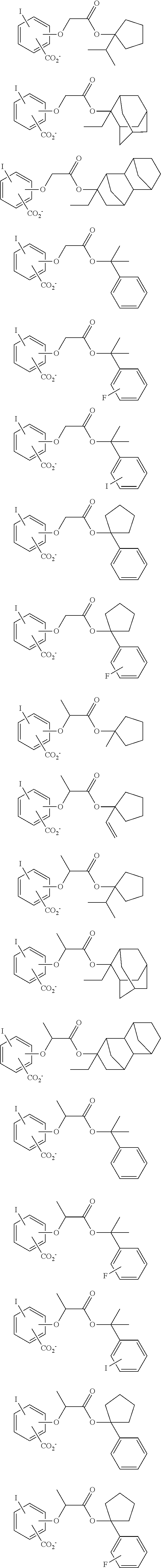

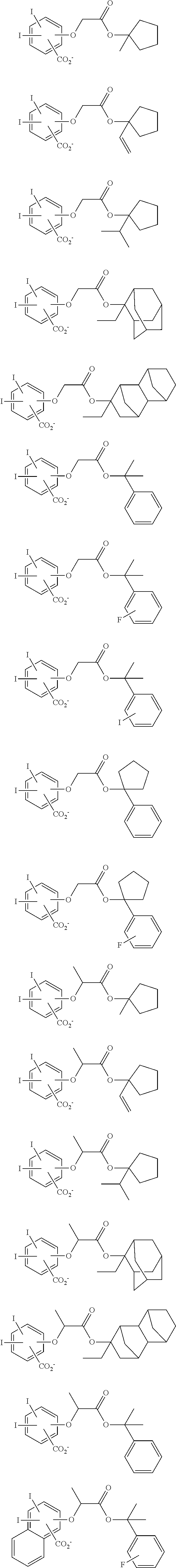

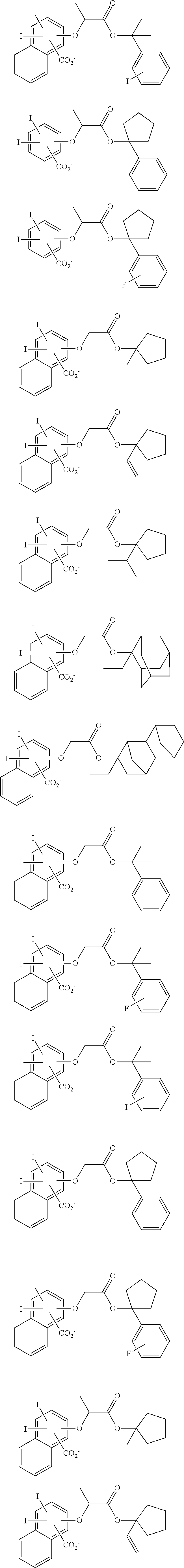

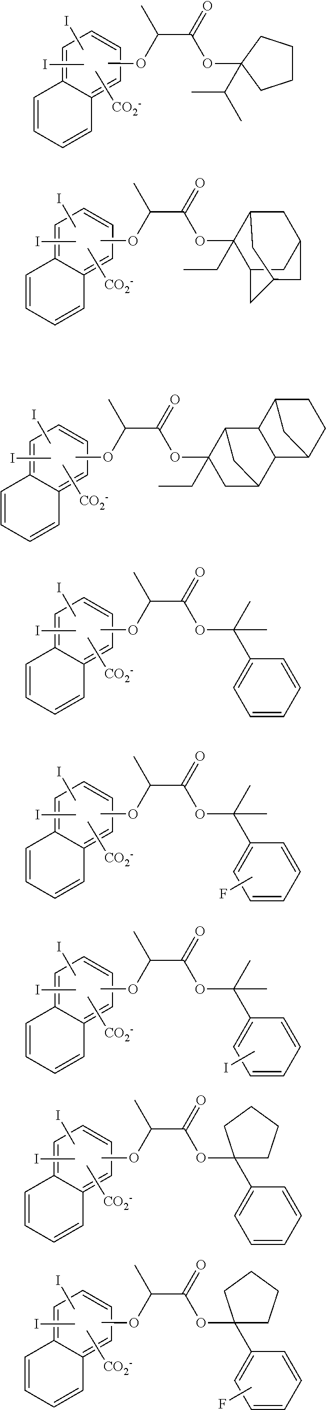

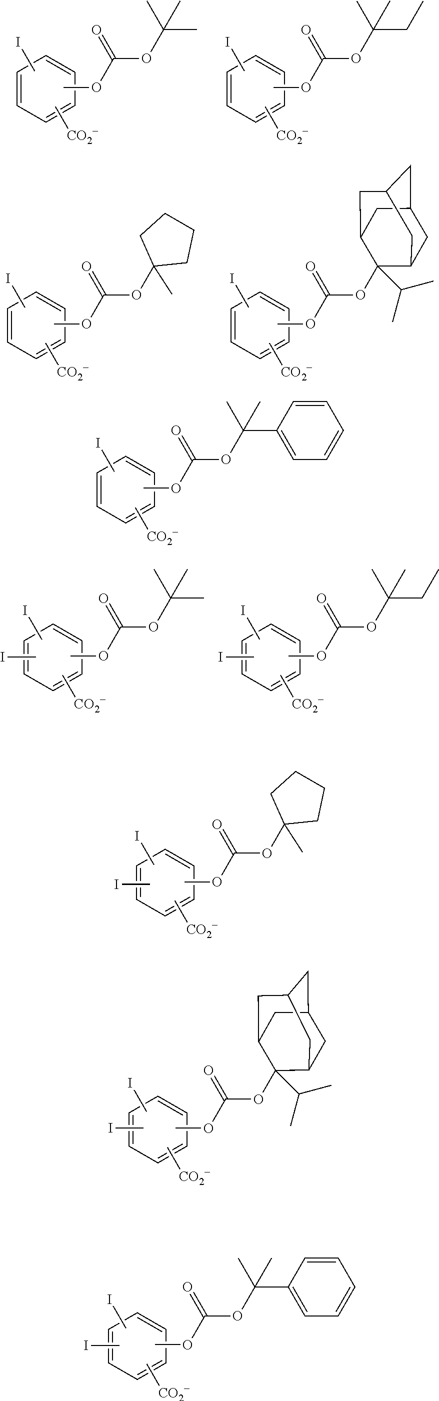

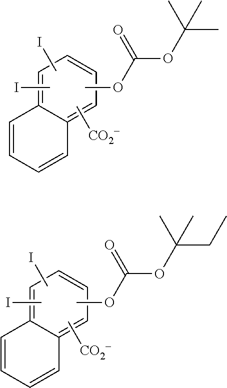

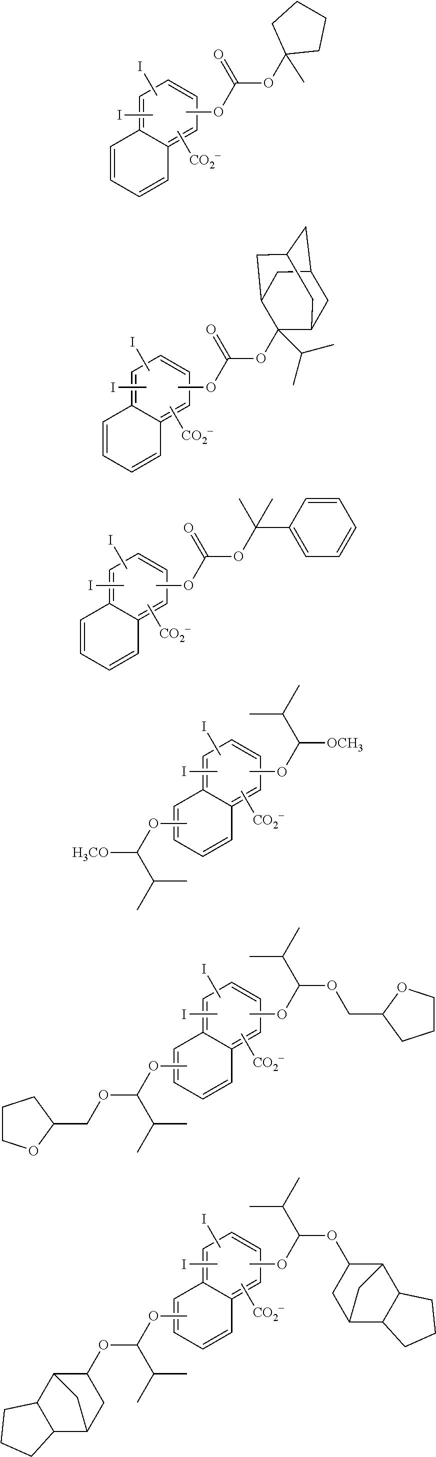

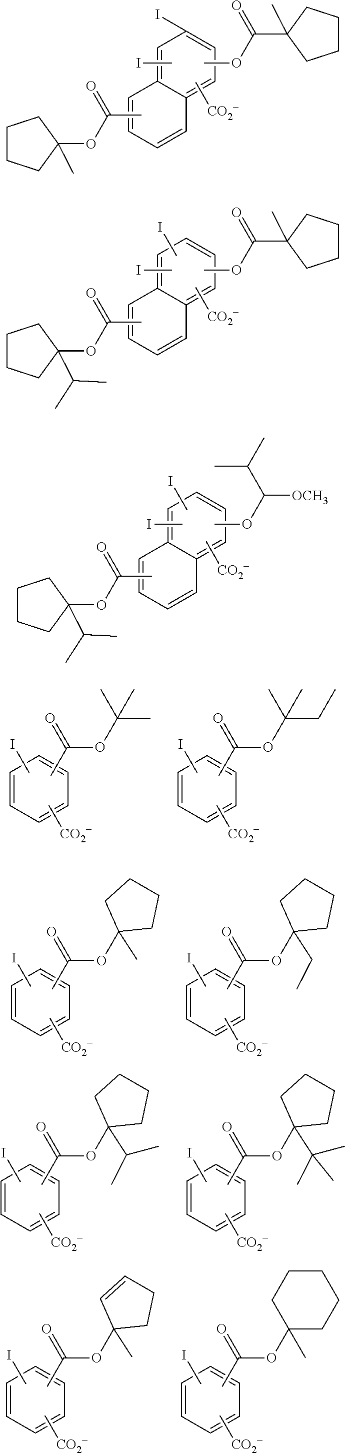

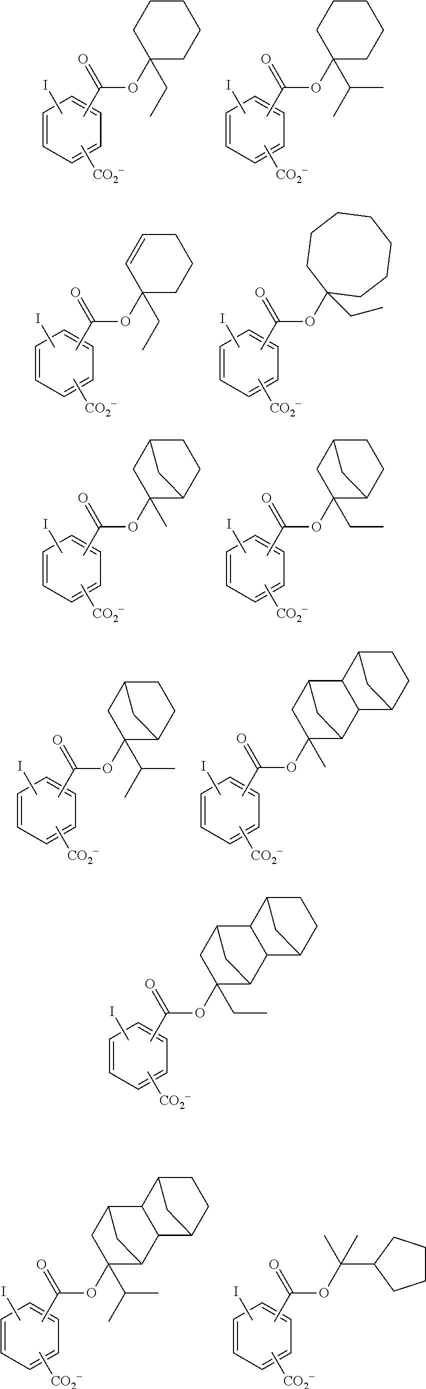

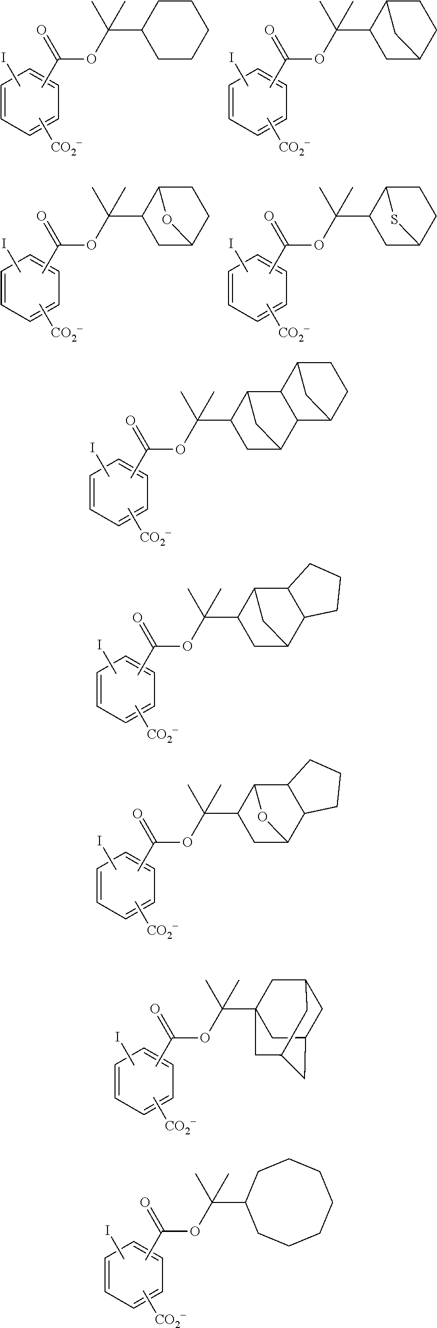

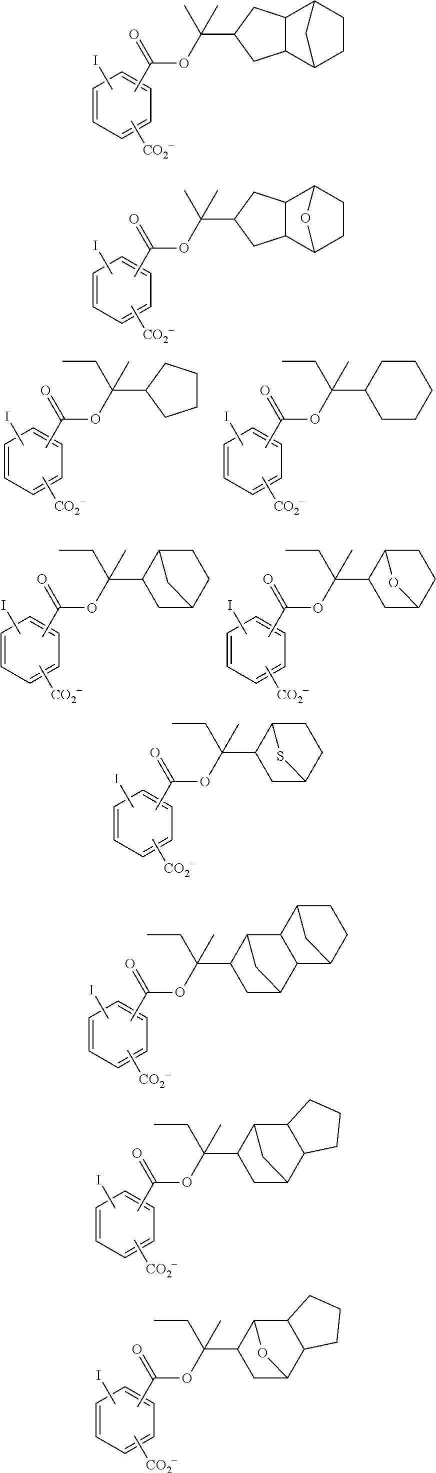

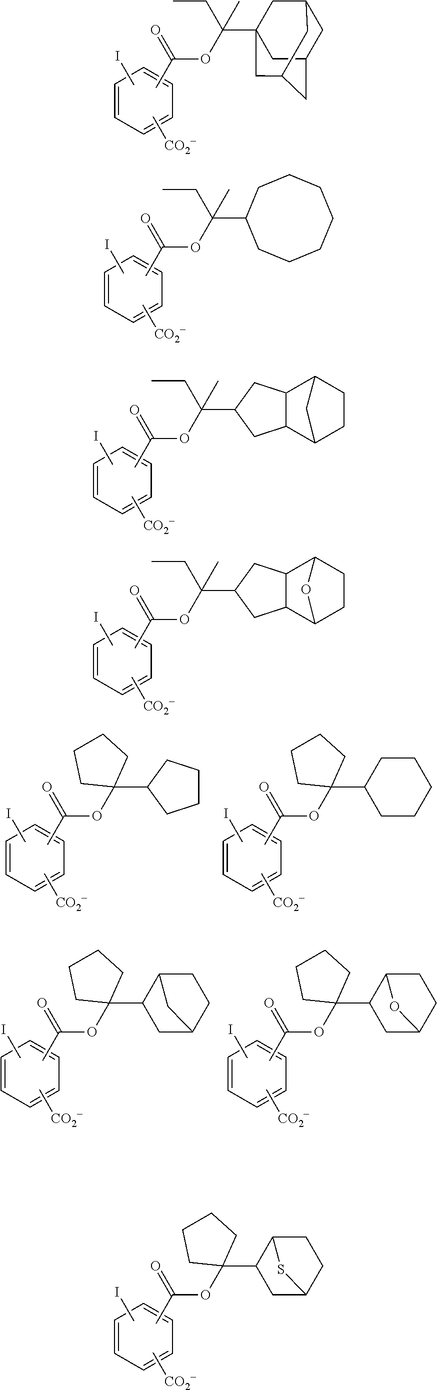

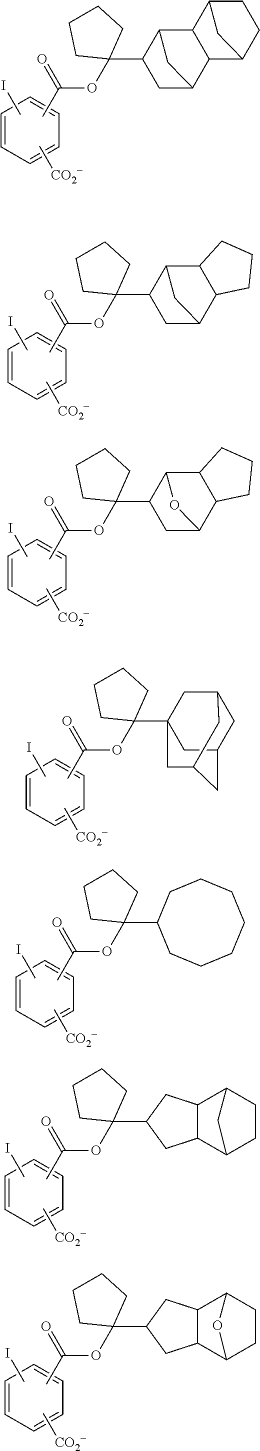

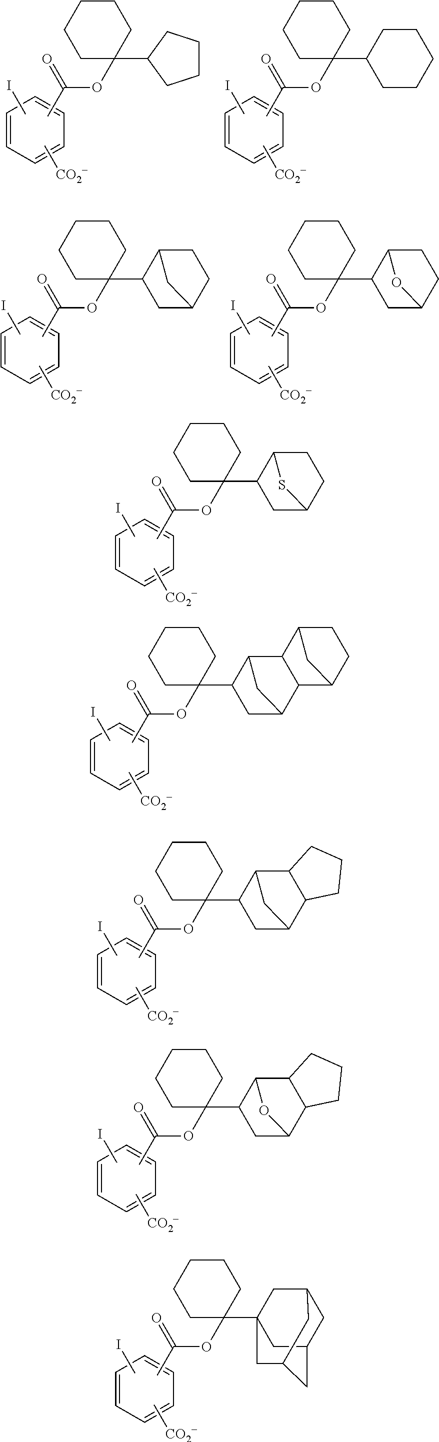

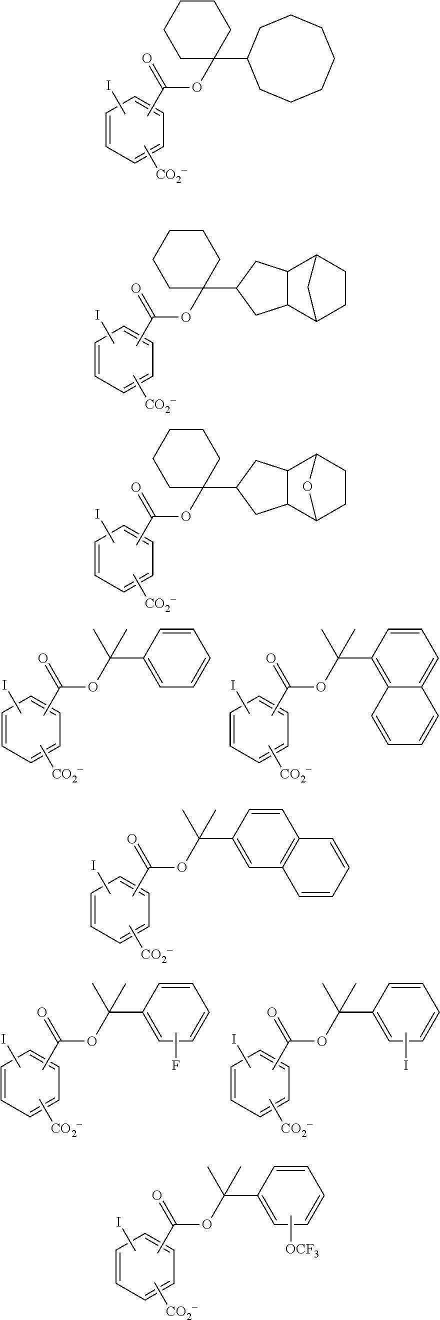

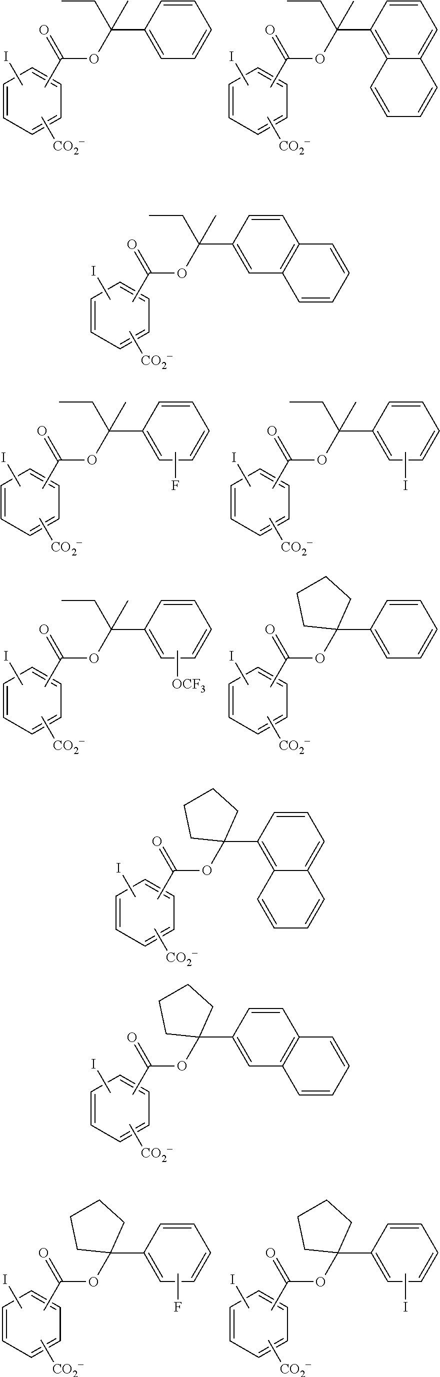

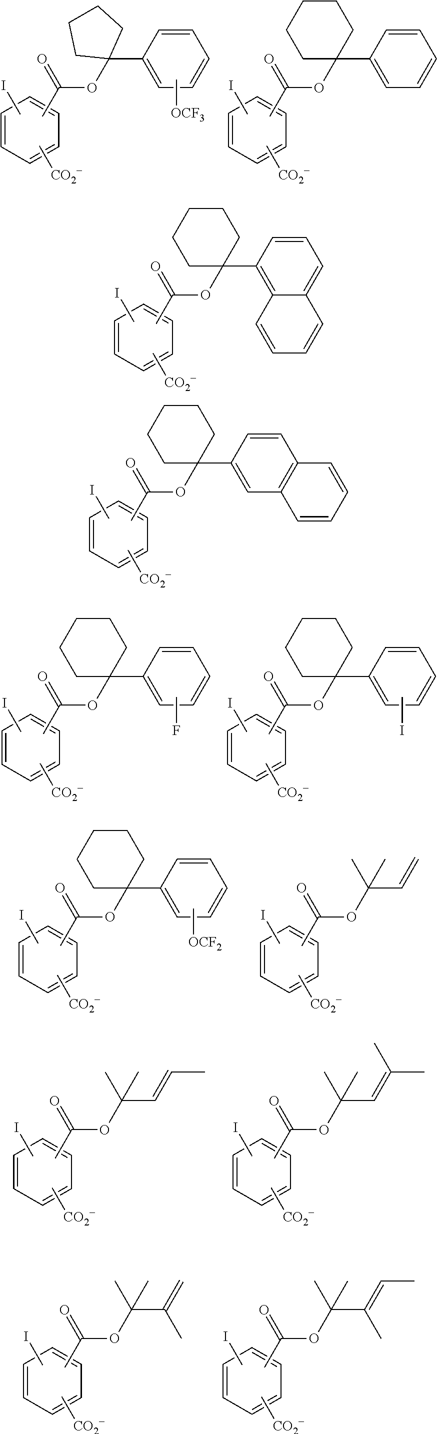

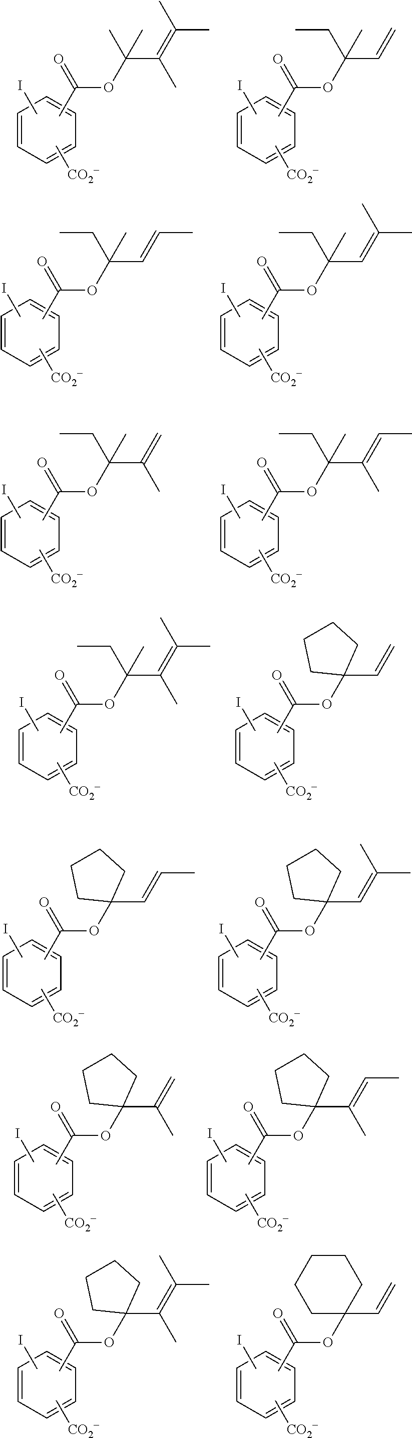

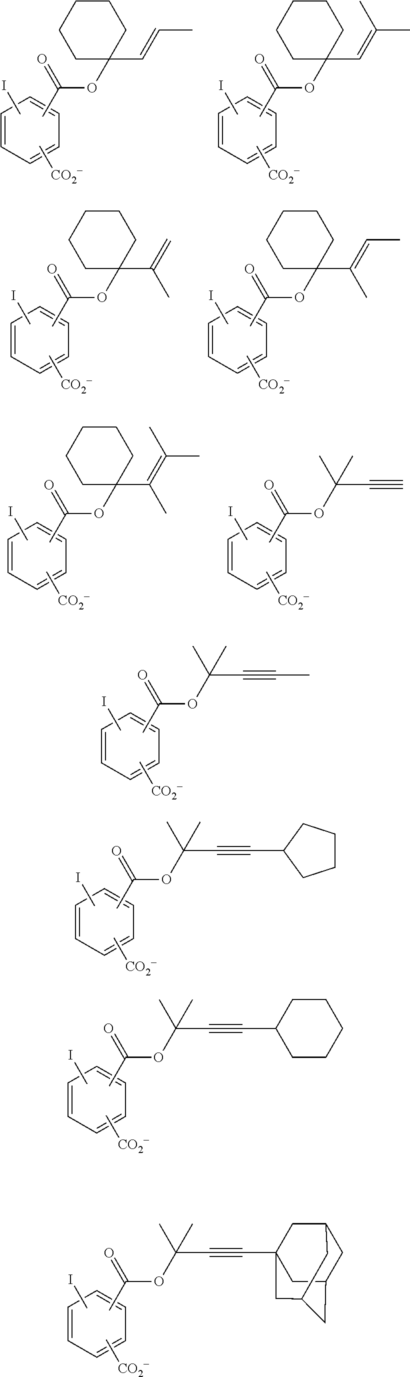

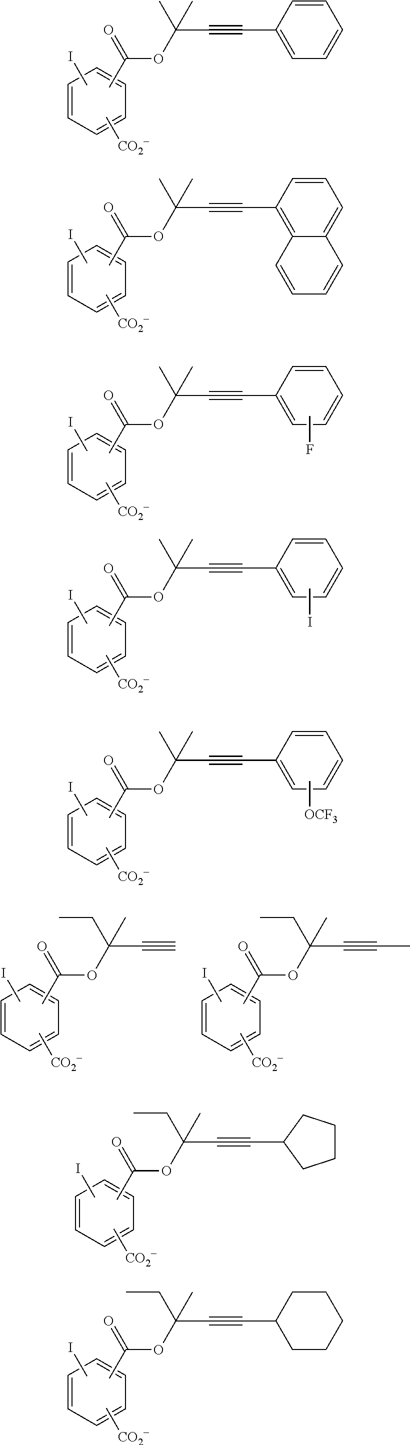

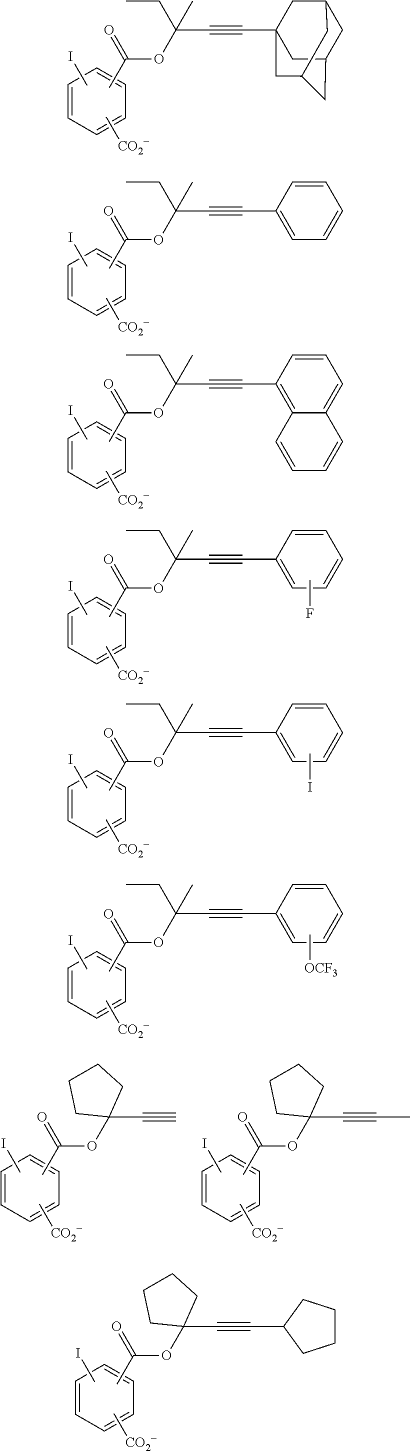

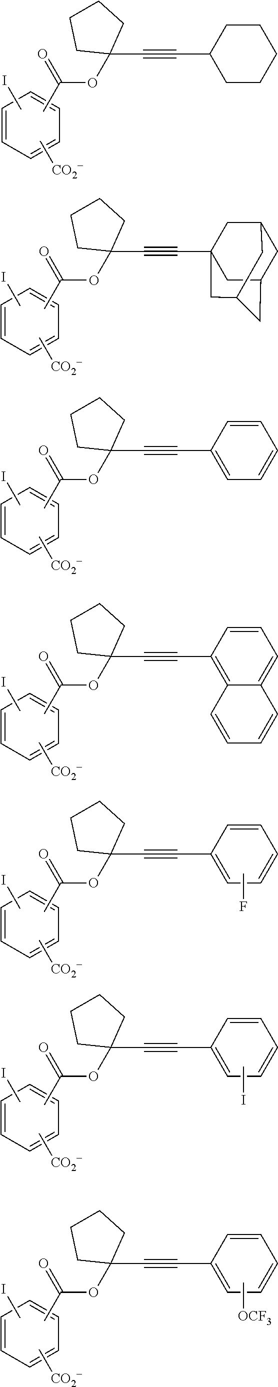

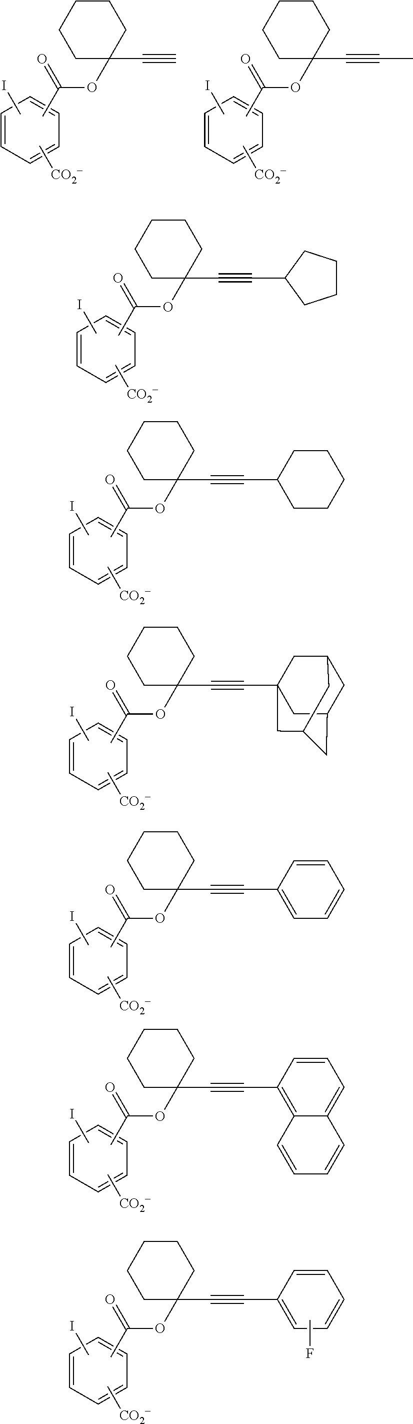

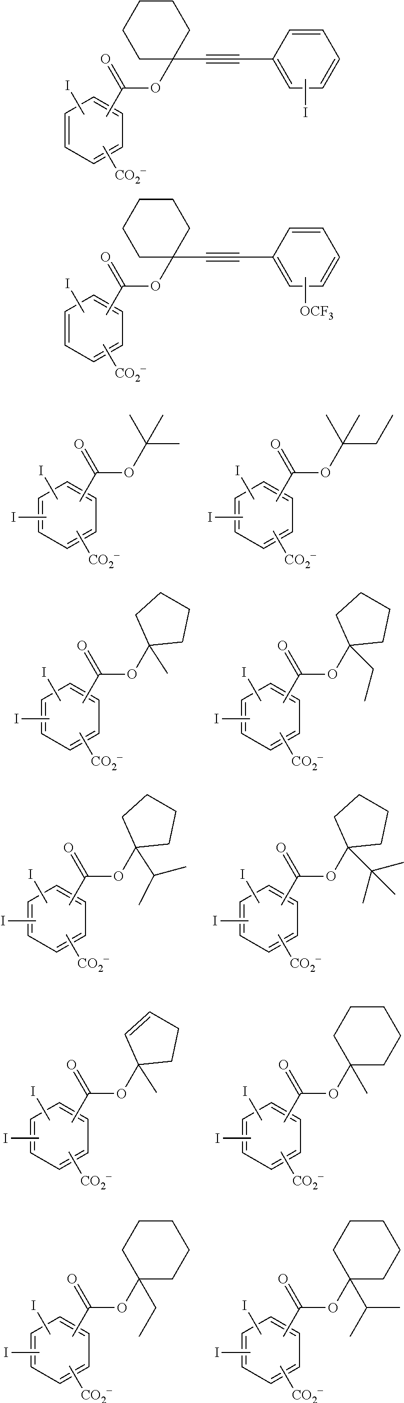

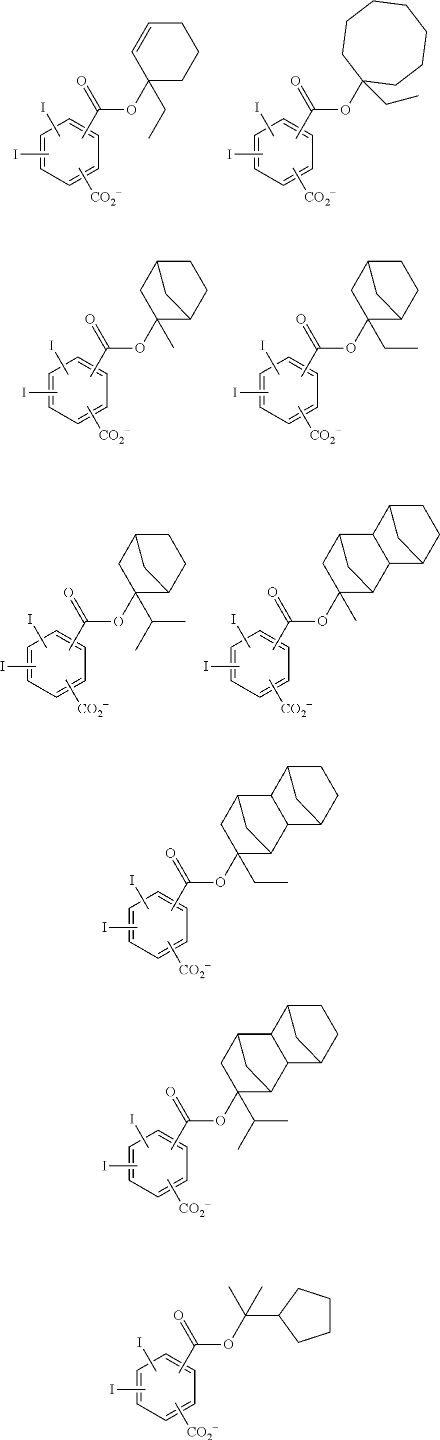

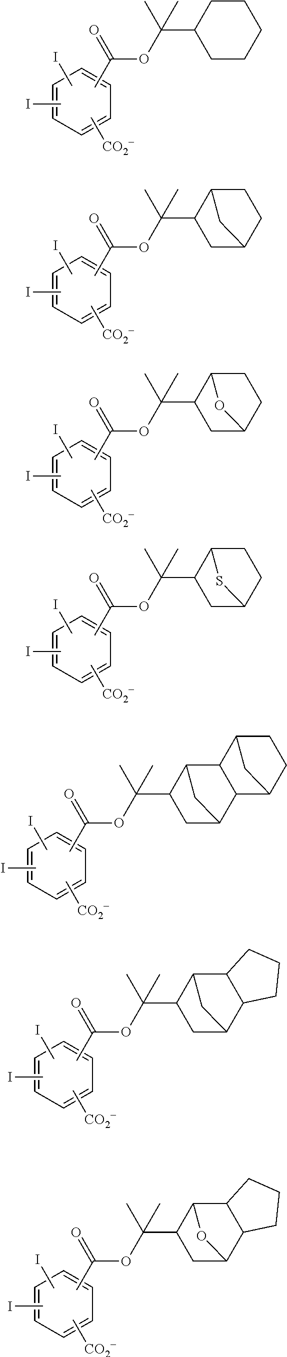

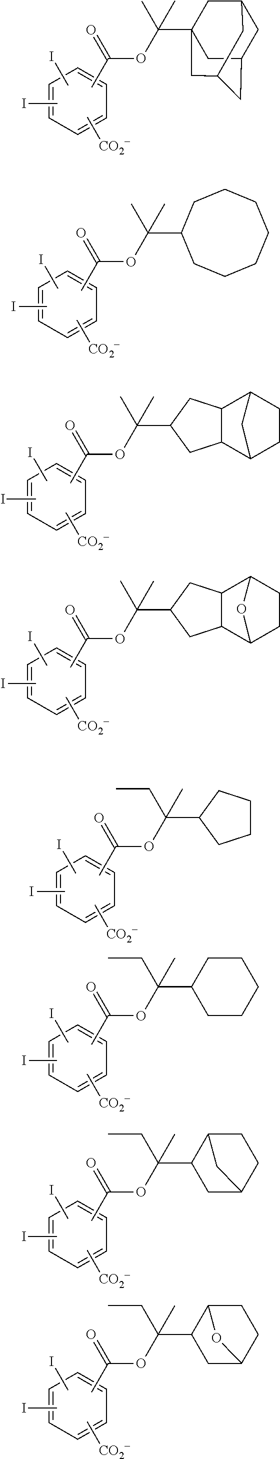

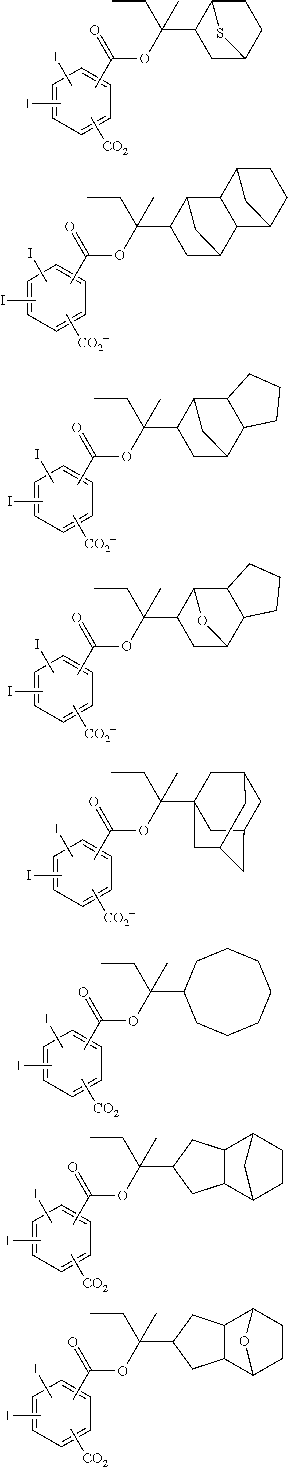

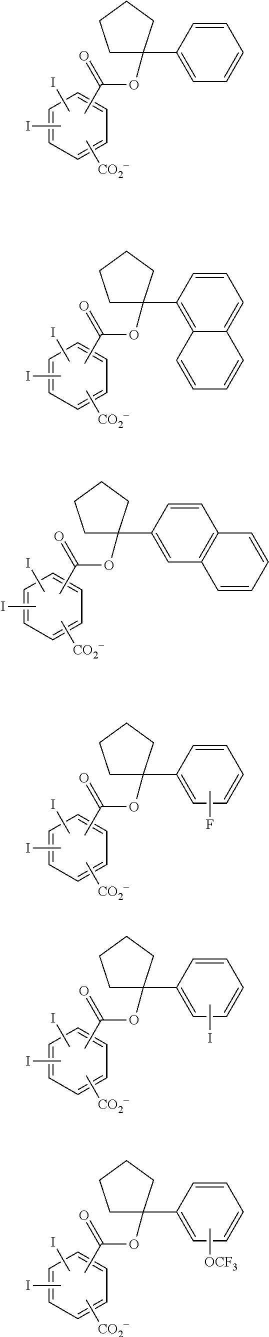

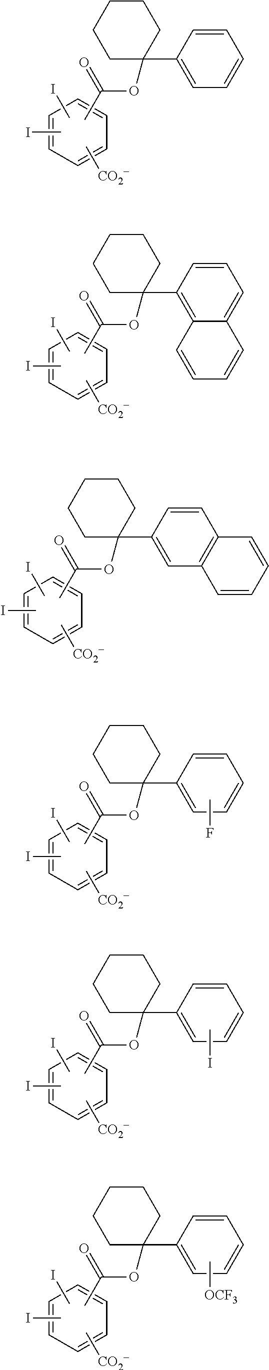

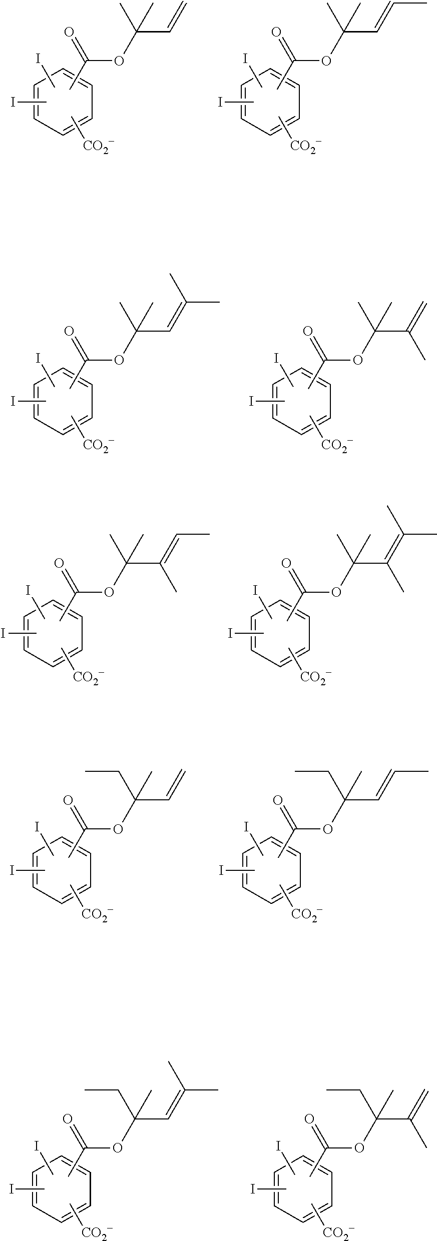

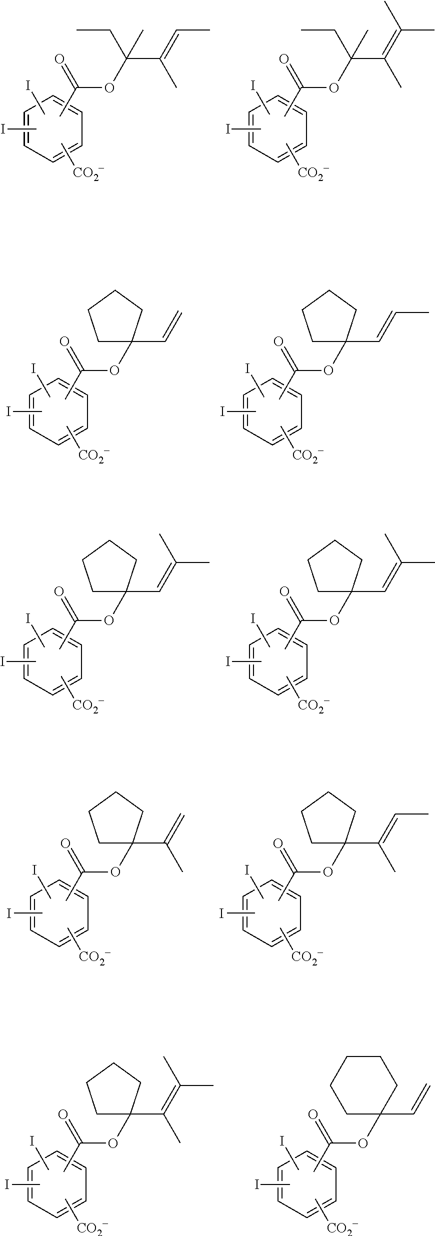

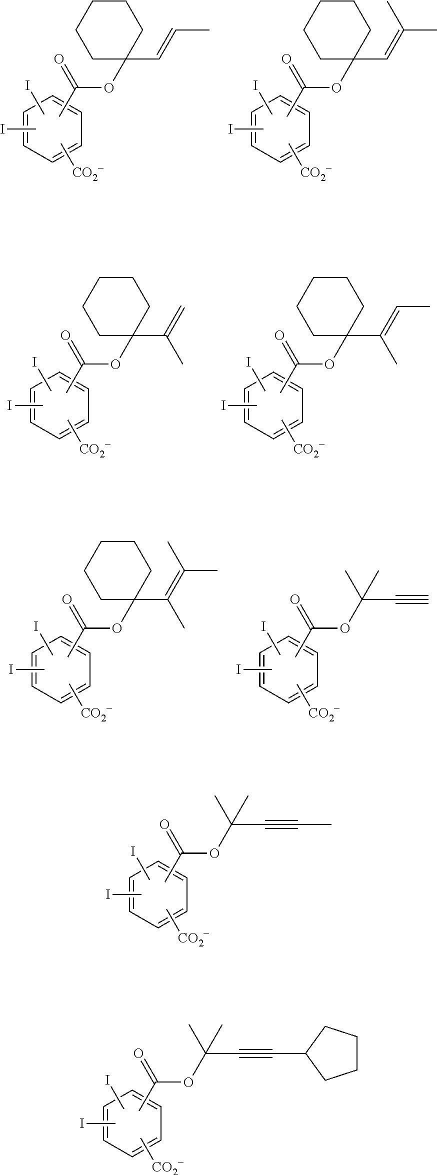

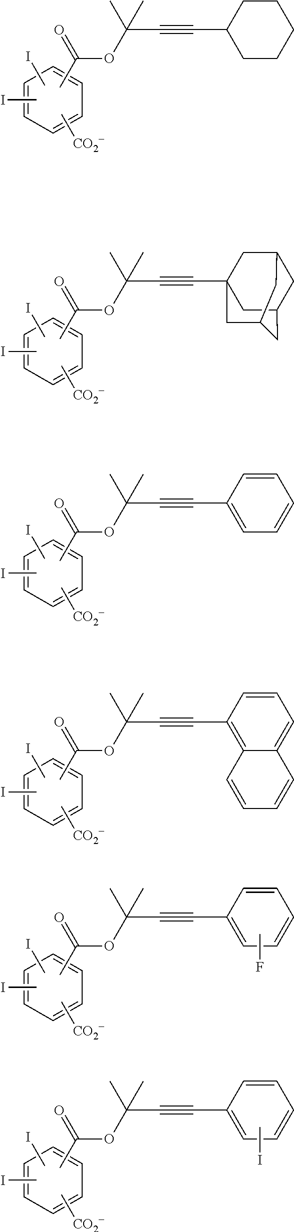

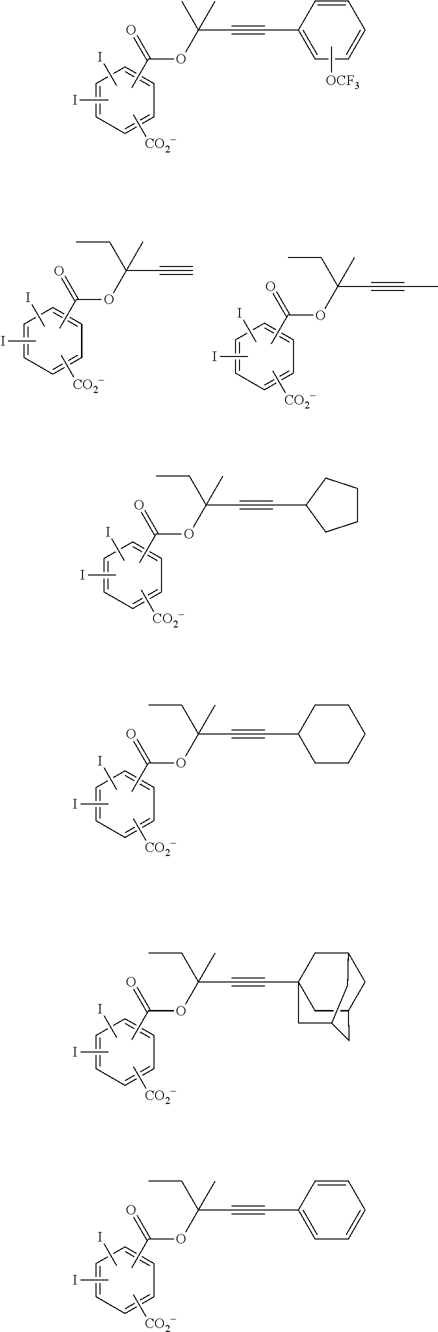

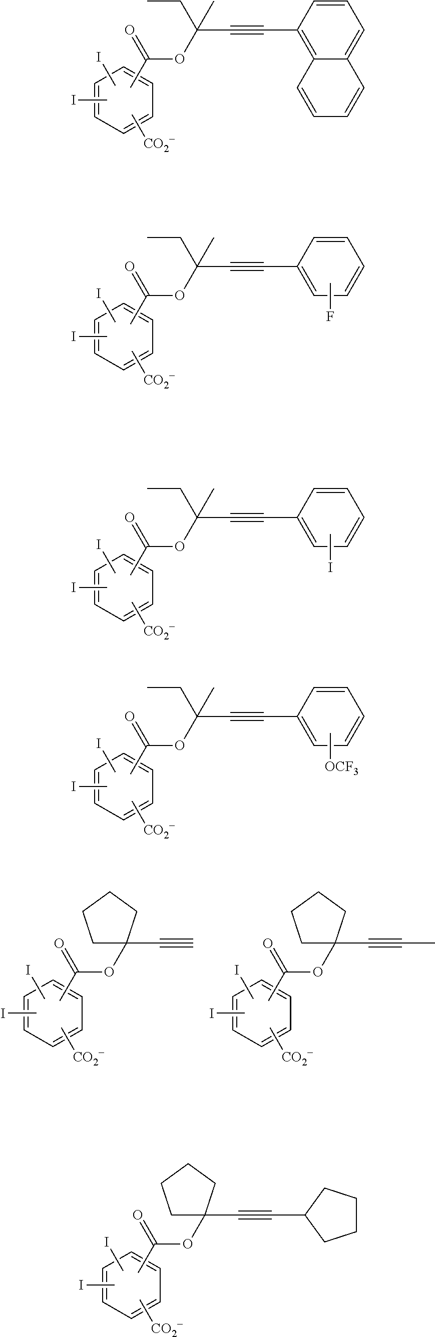

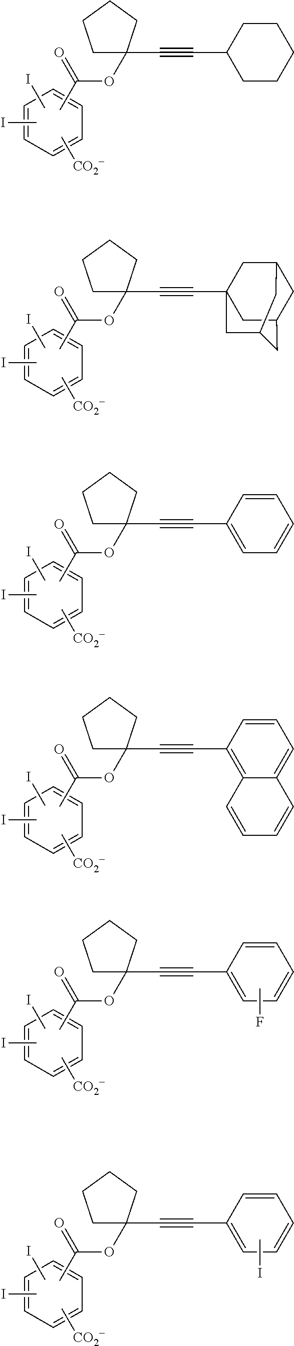

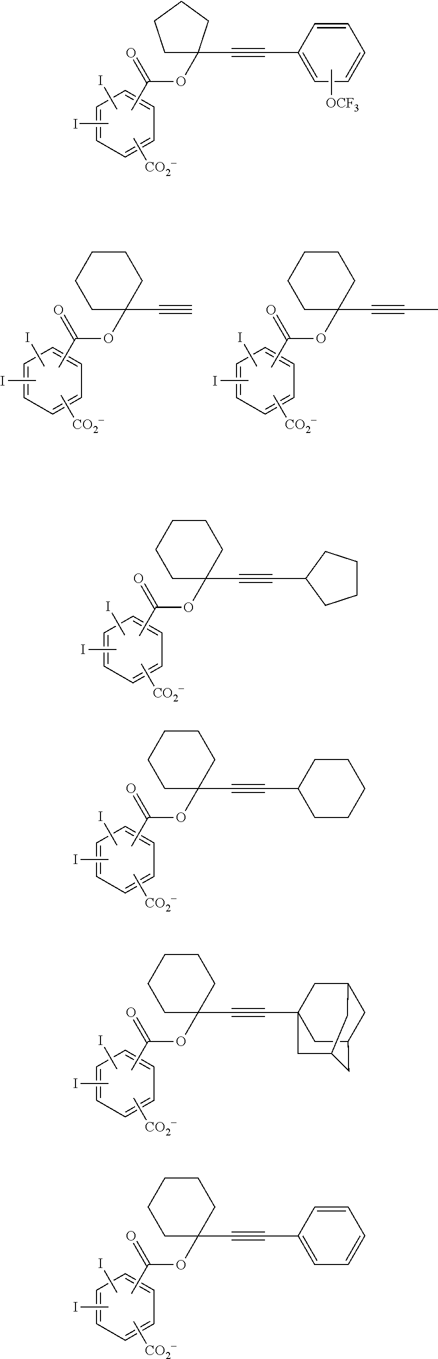

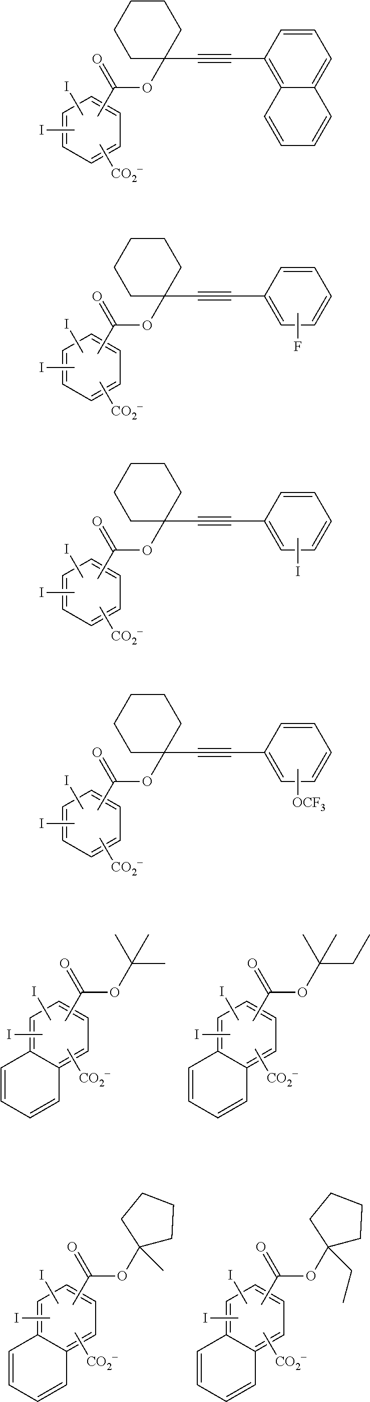

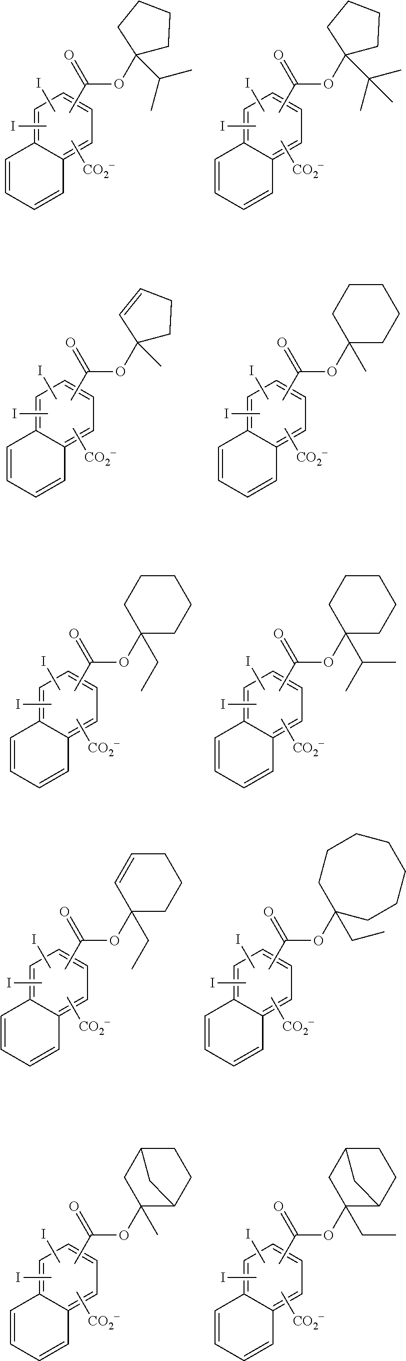

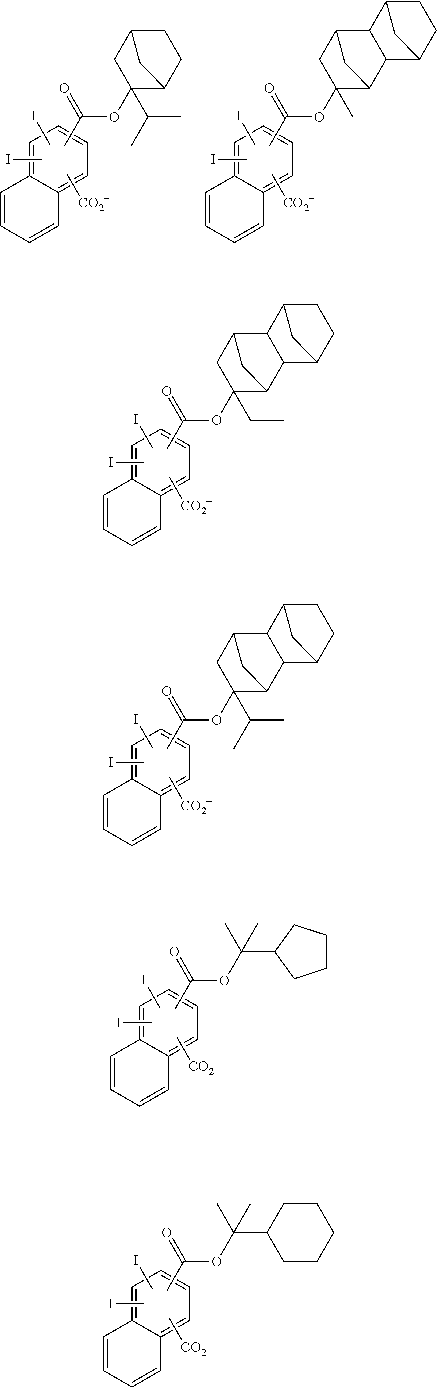

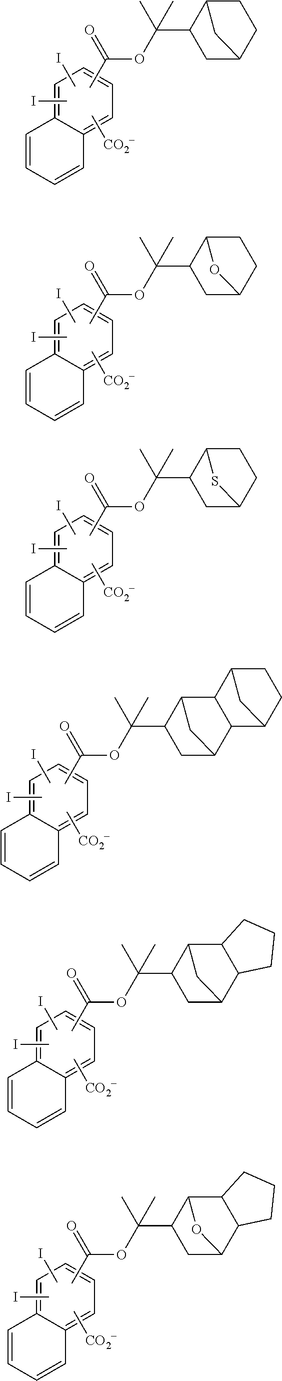

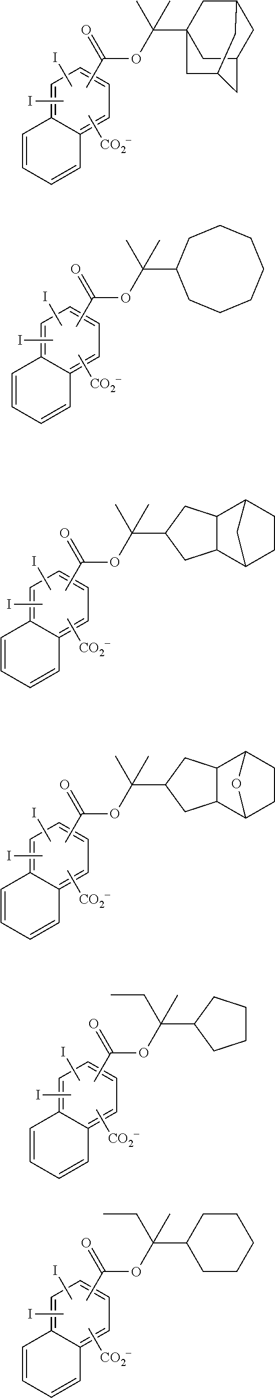

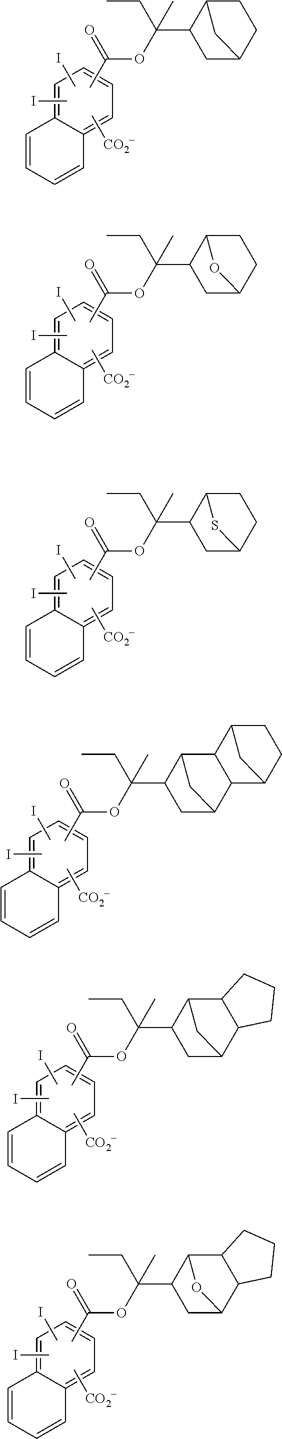

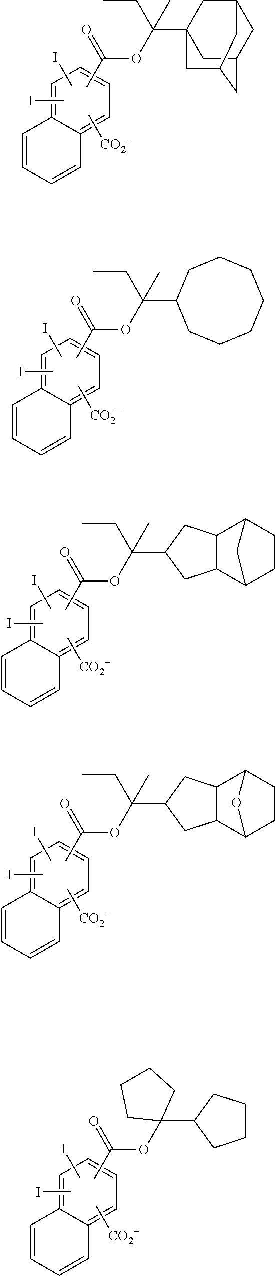

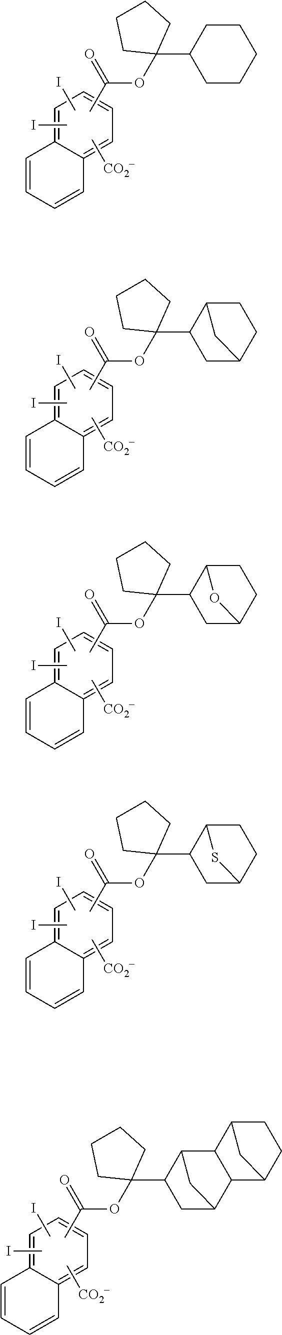

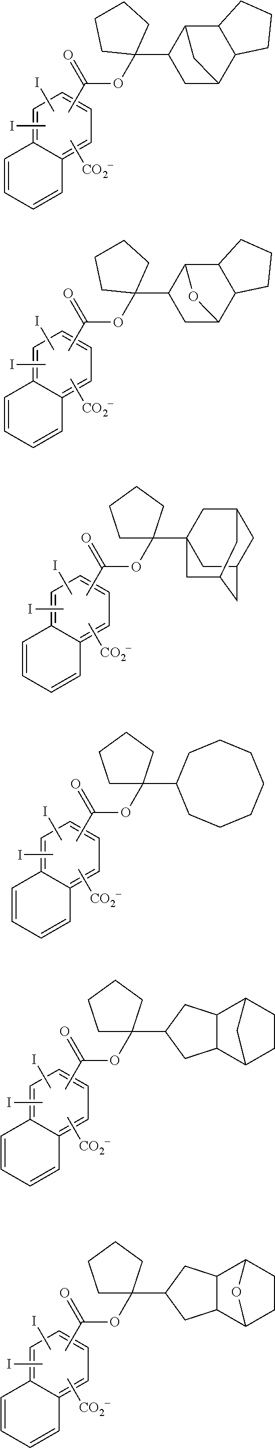

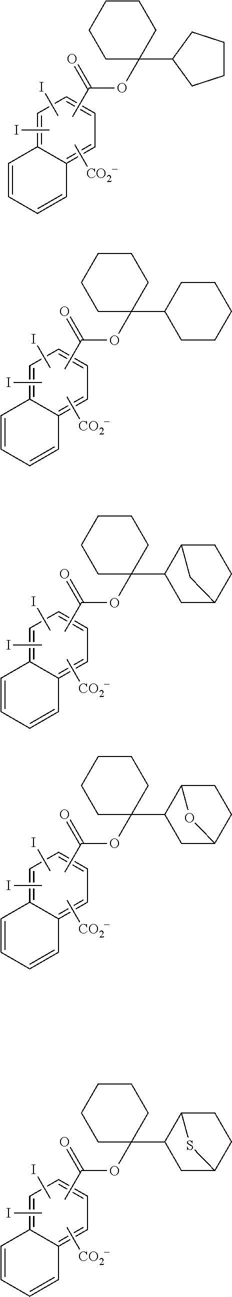

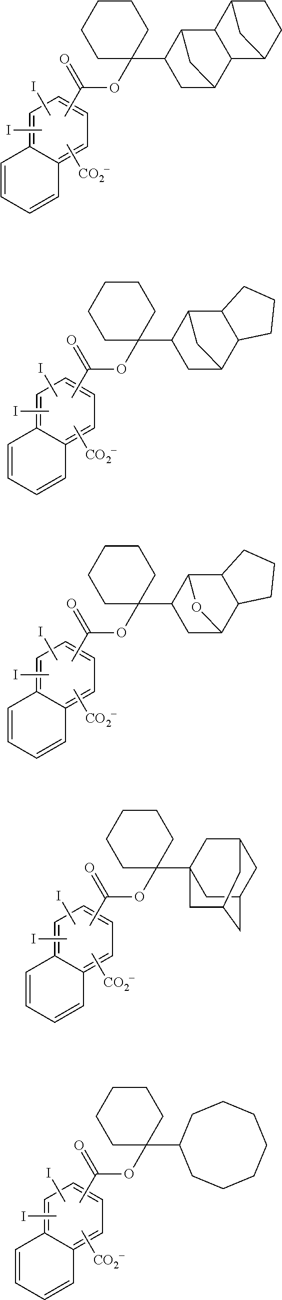

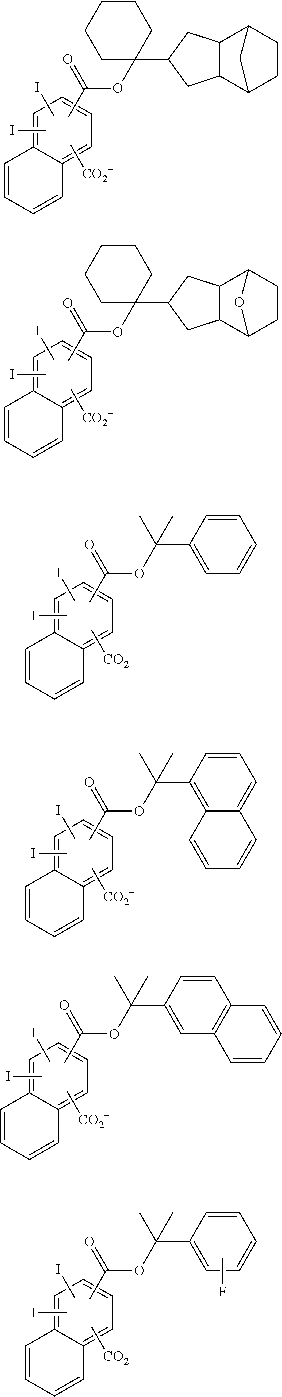

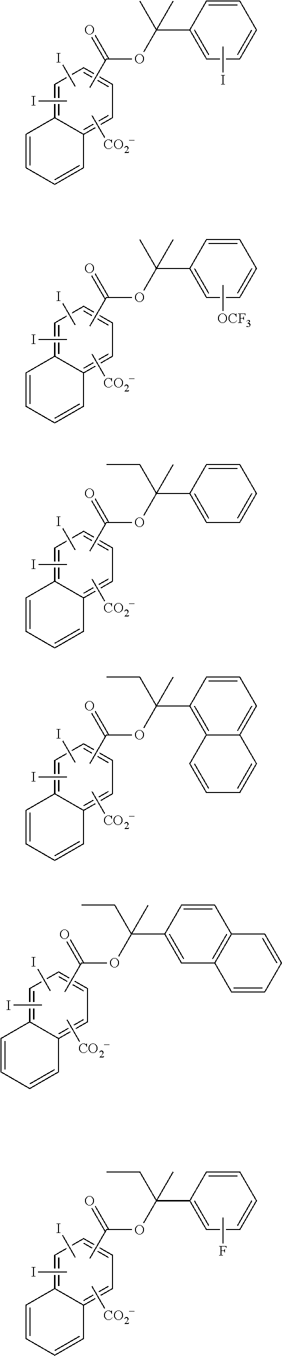

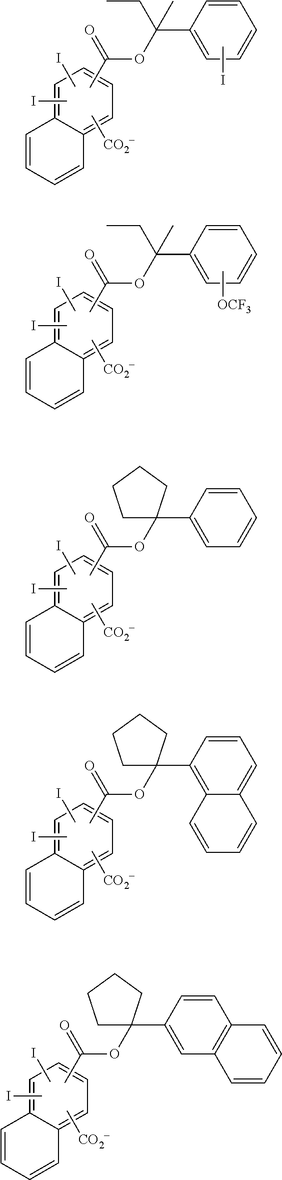

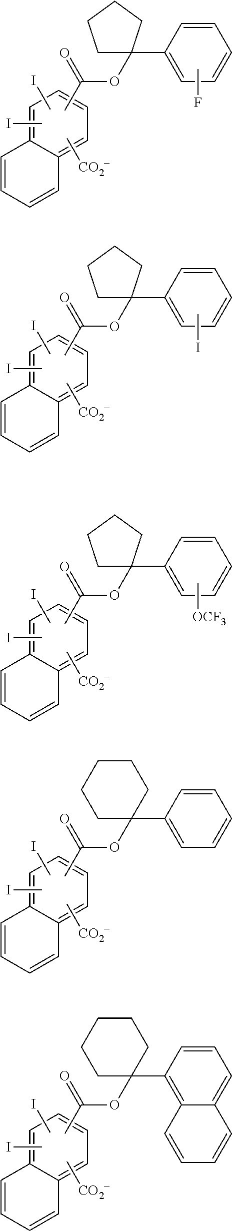

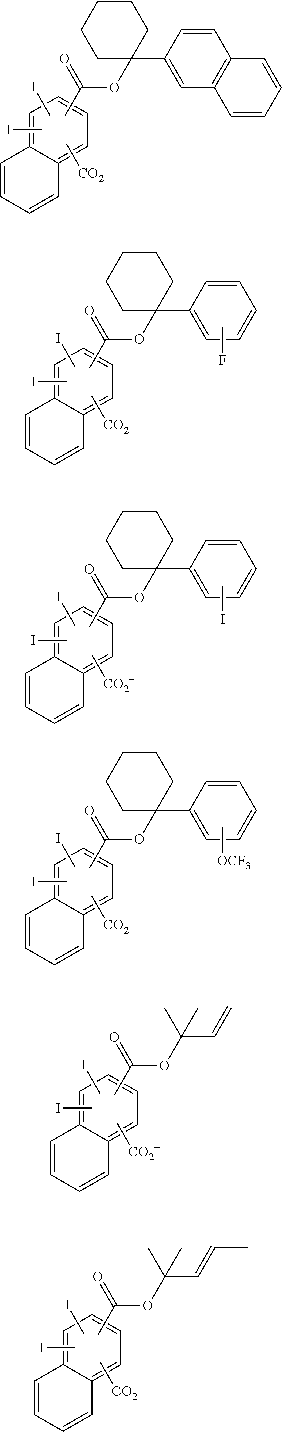

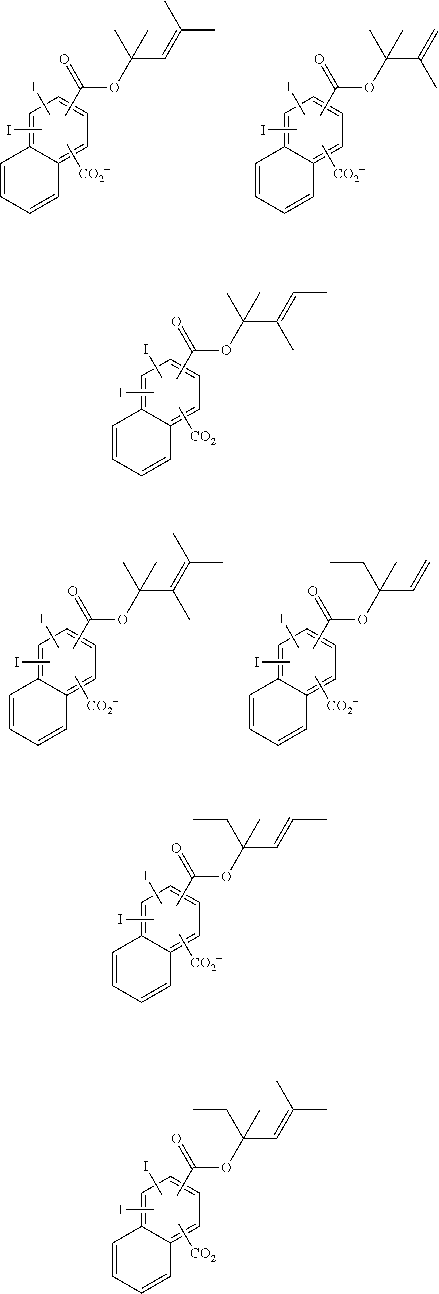

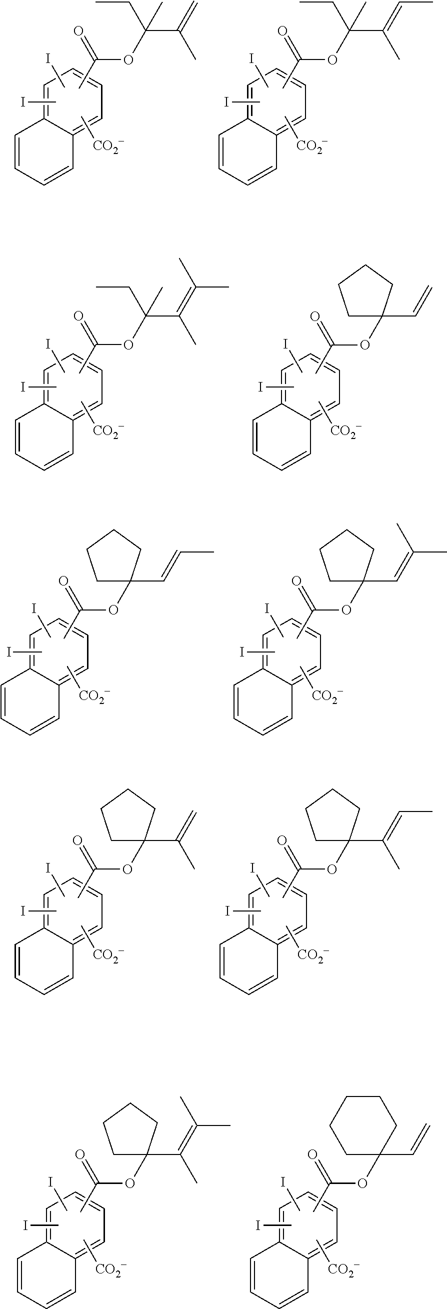

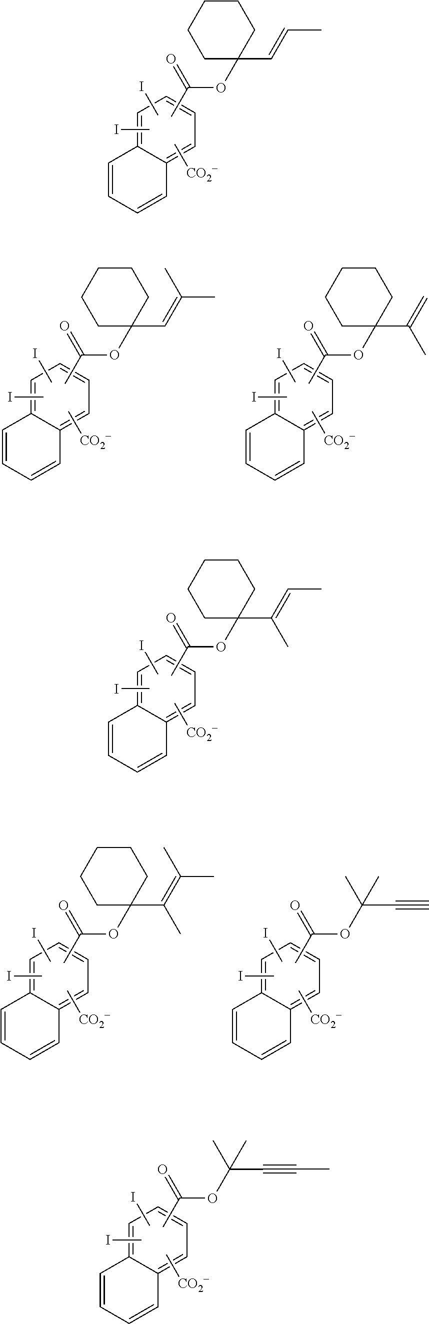

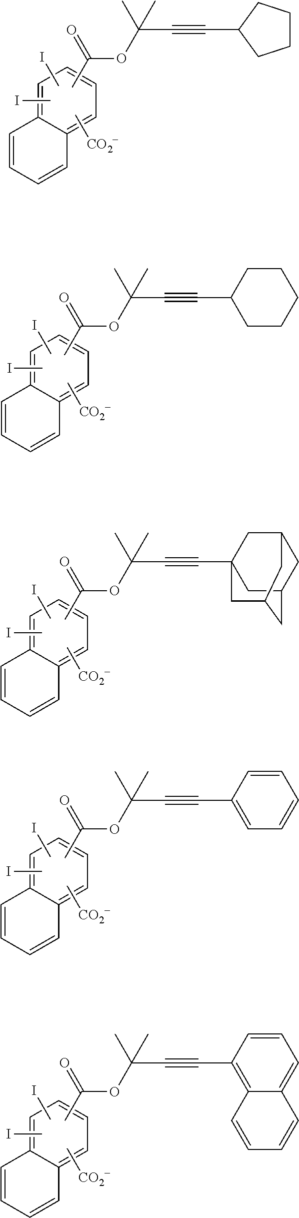

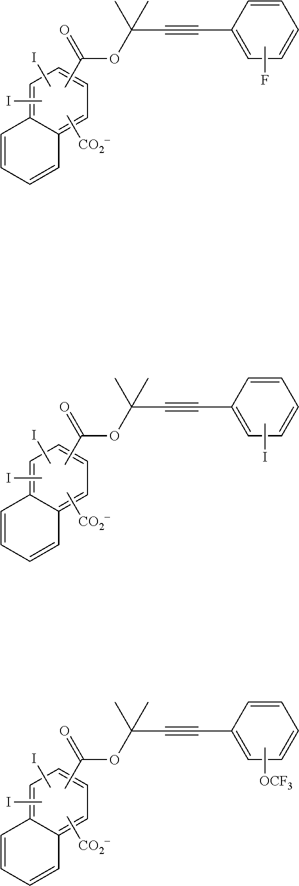

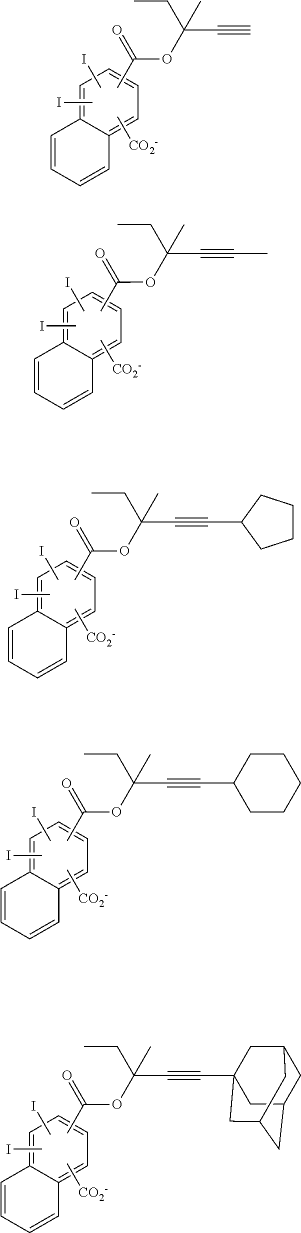

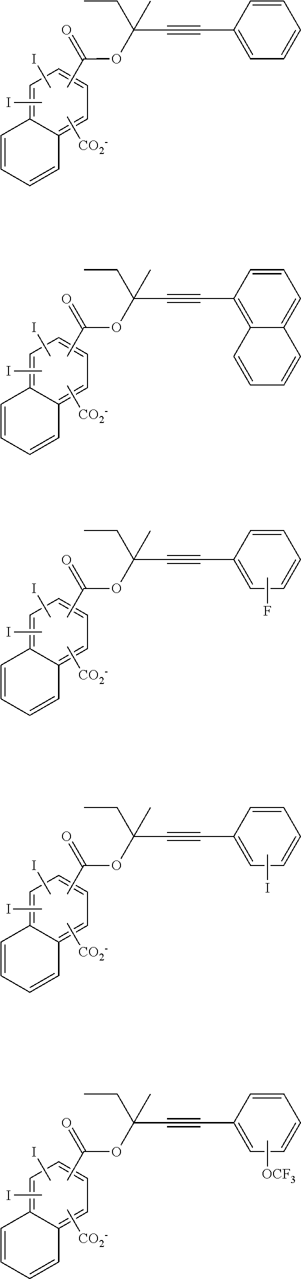

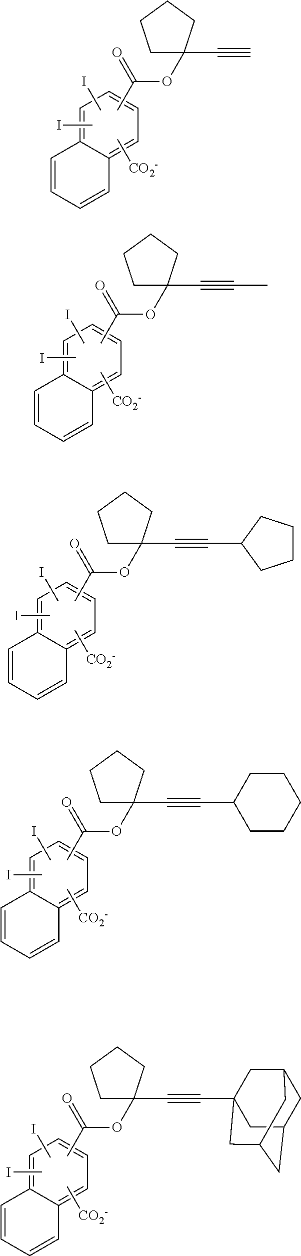

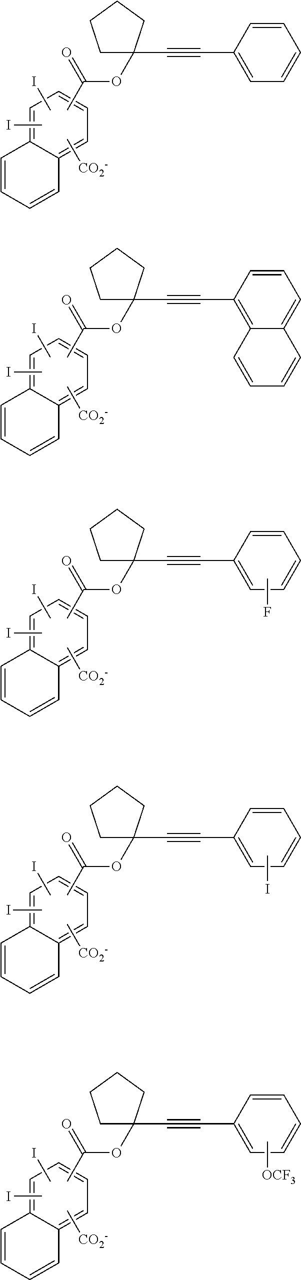

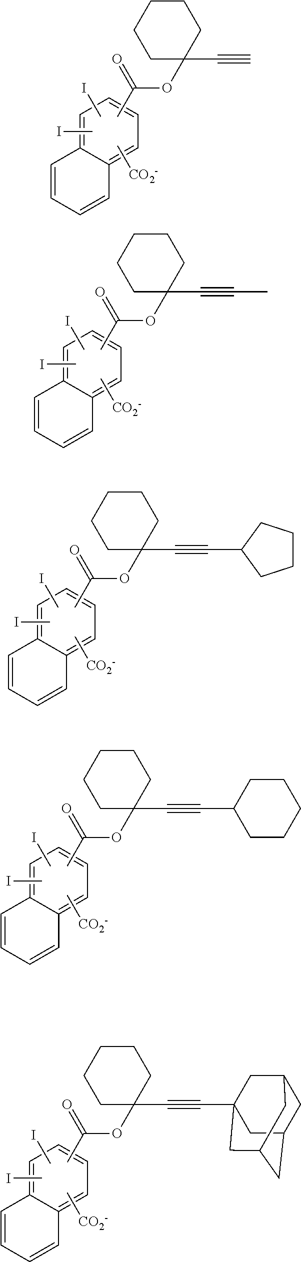

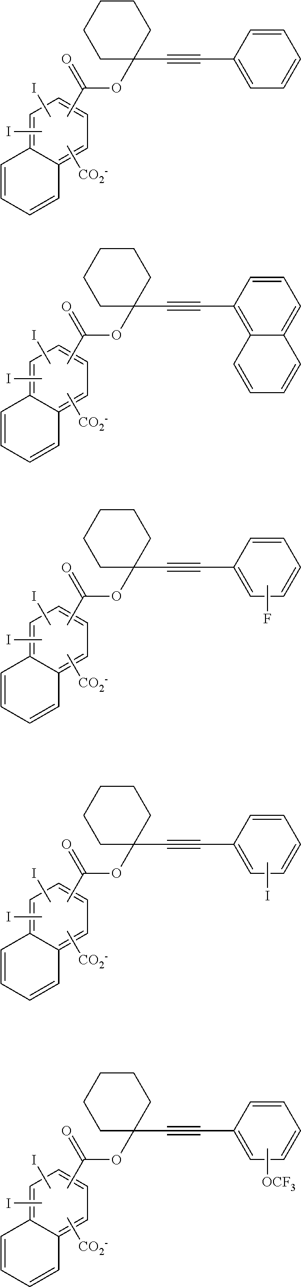

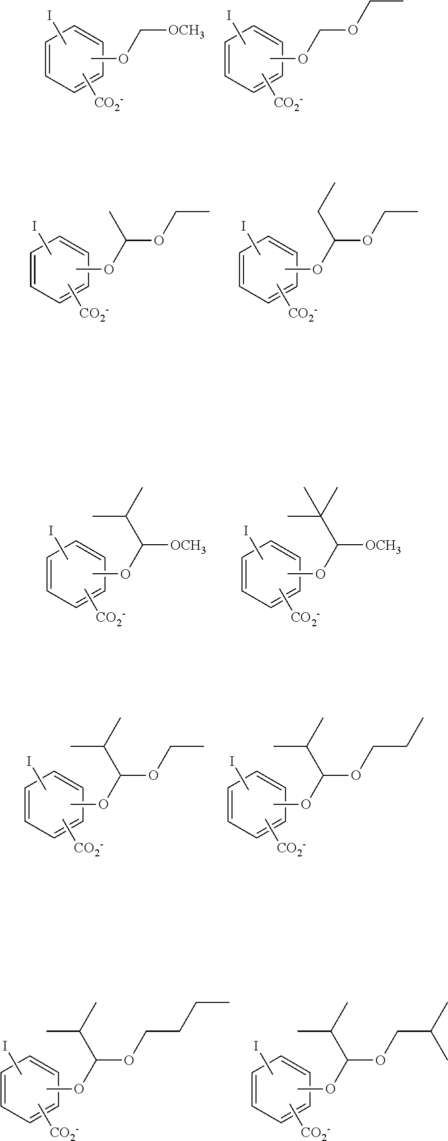

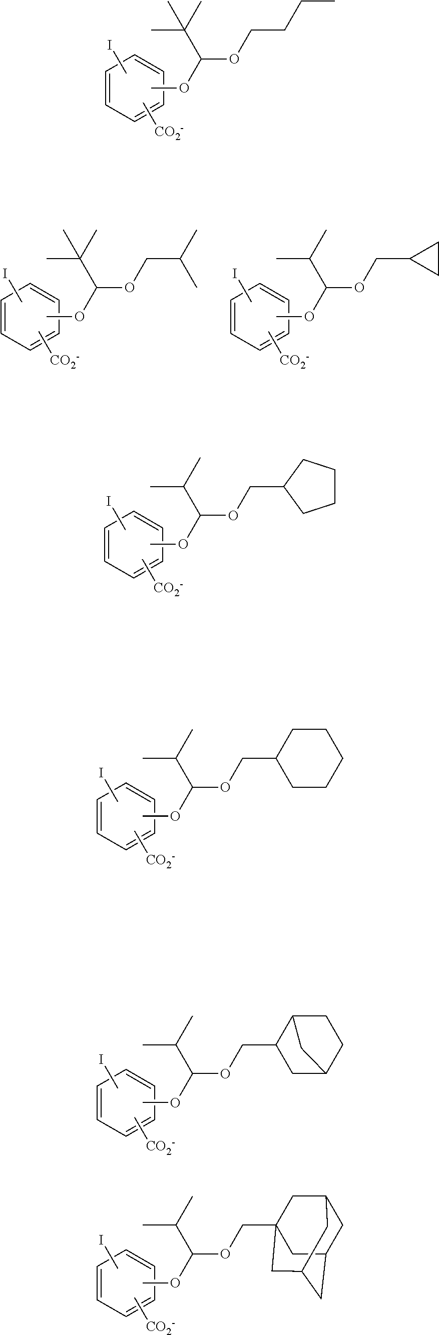

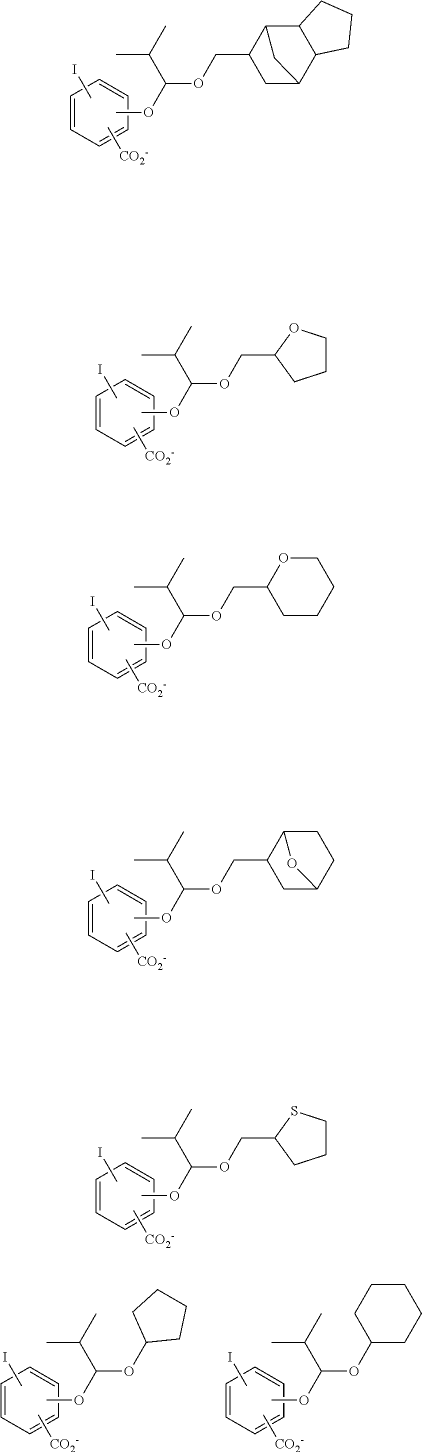

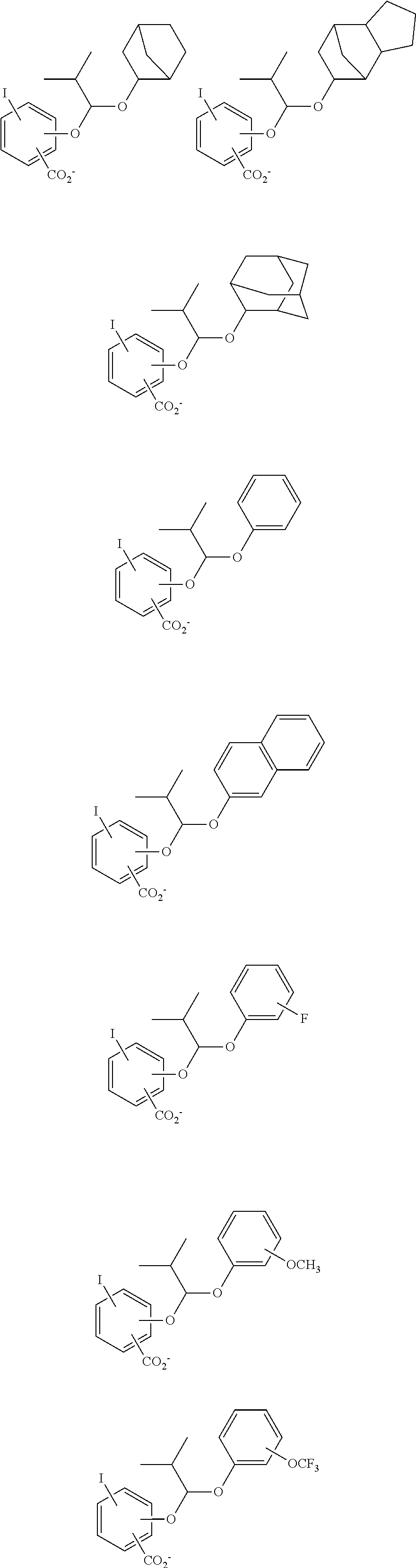

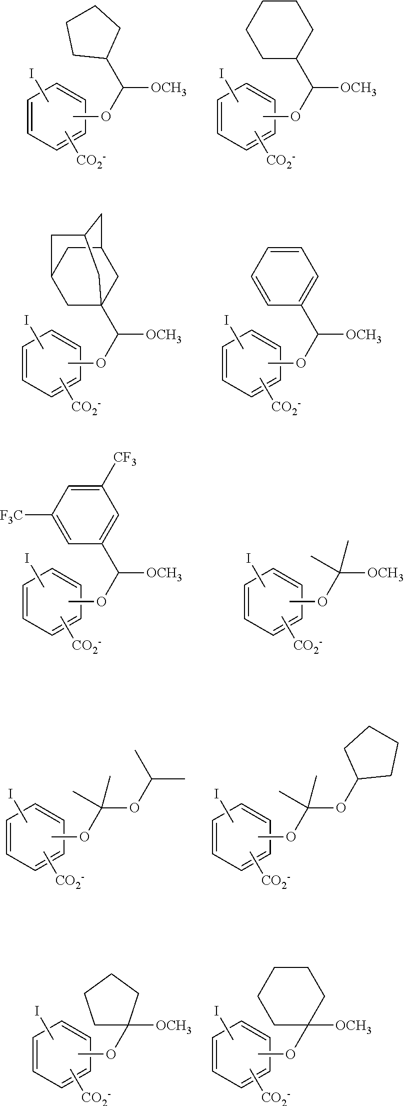

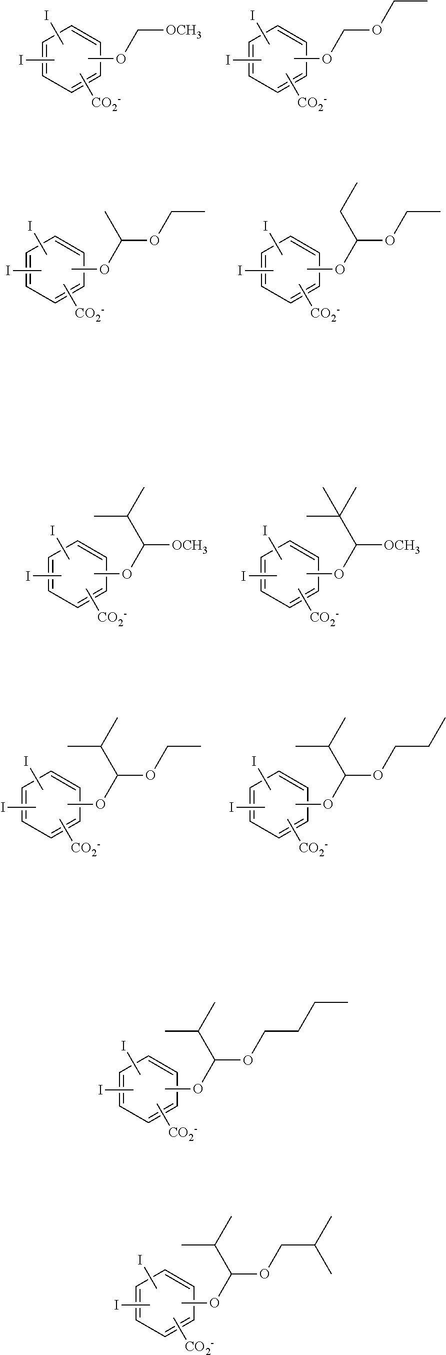

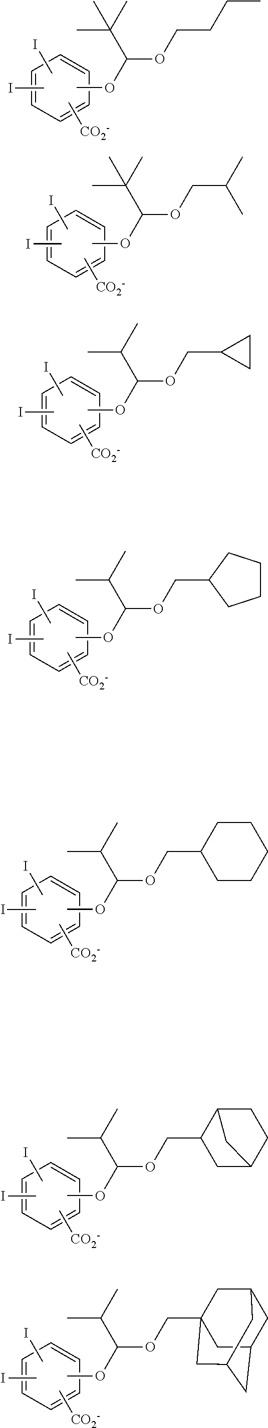

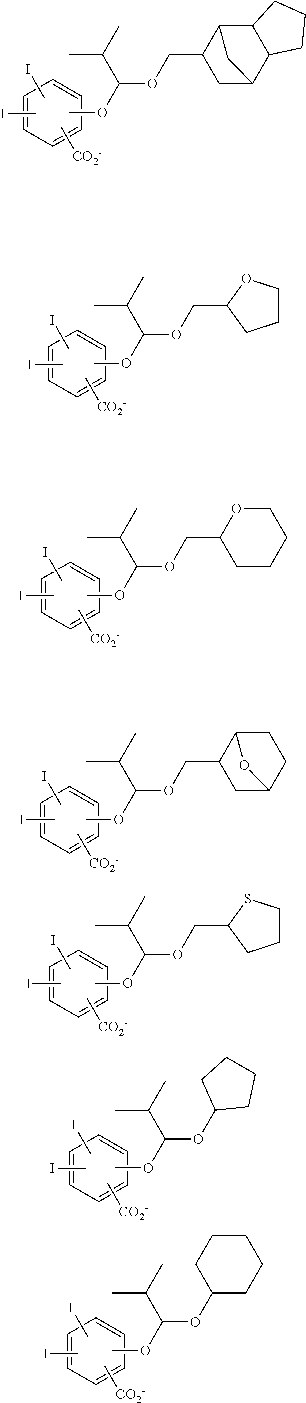

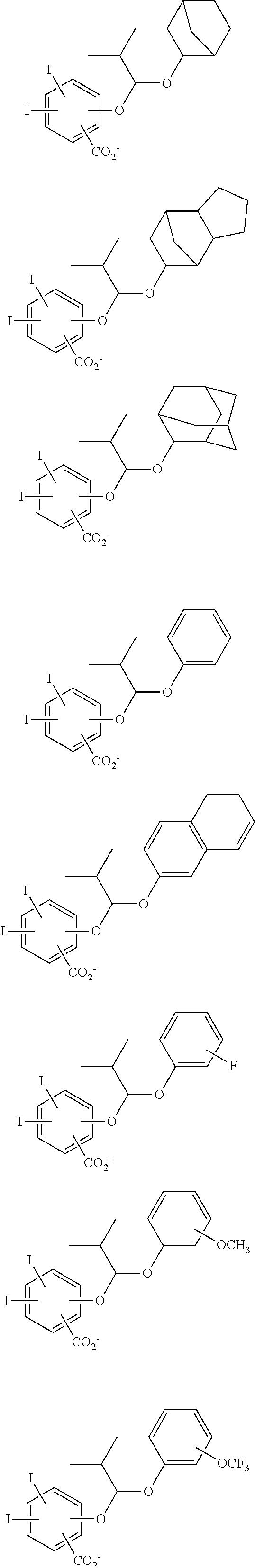

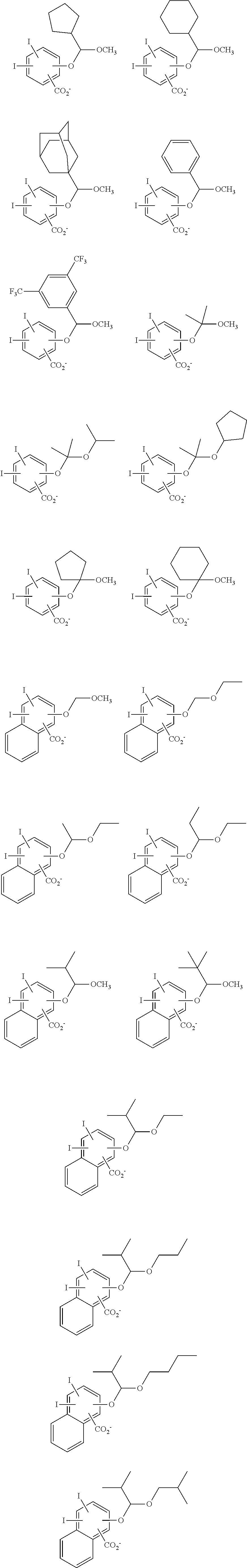

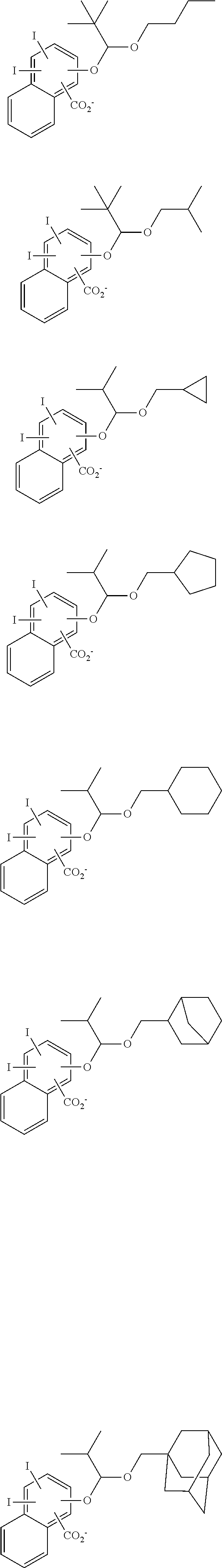

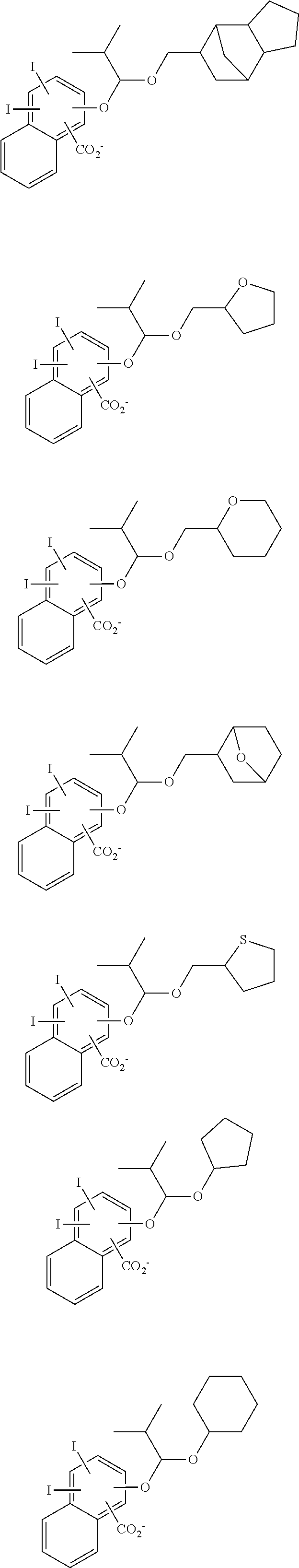

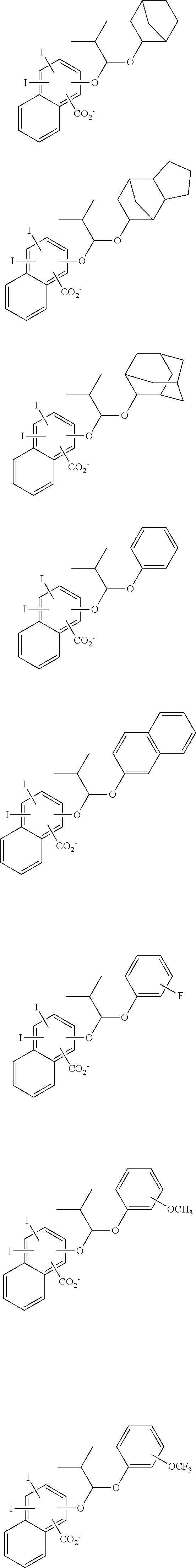

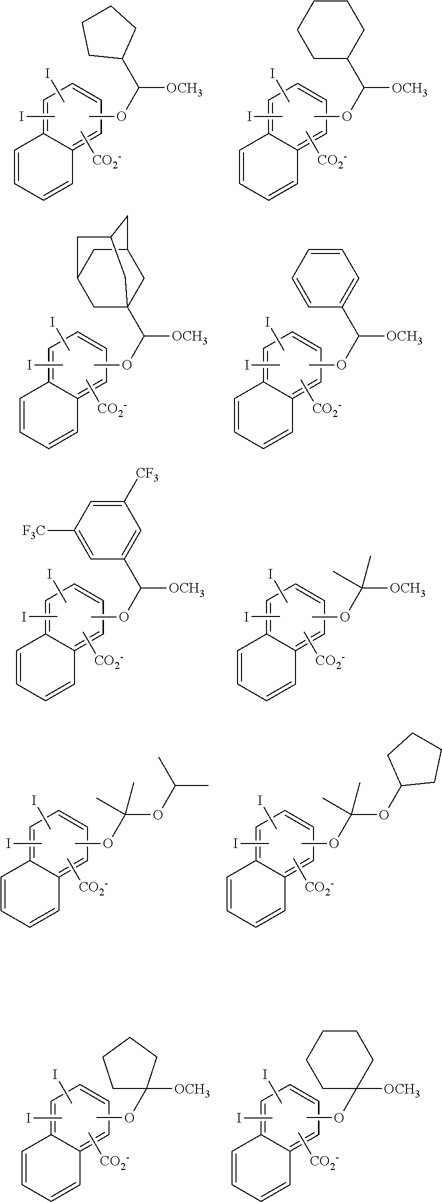

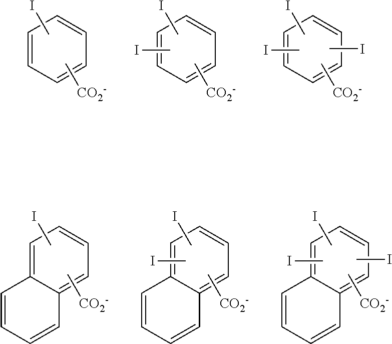

Examples of the onium salt include arbitrary combinations of anions with cations, both as exemplified above.

The onium salt can be synthesized by a suitable method as described in JP-A 2020-091312 (U.S. Pat. No. 11,586,111), although the synthesis method is not limited thereto.

The onium salt of the invention is structurally characterized as consisting of a sulfonium cation having a nitro group and an aromatic carboxylic acid anion having an iodine atom. In the aromatic carboxylic acid anion containing iodine, iodine which is outstandingly absorptive to EUV of wavelength 13.5 nm generates secondary electrons during EUV exposure. Also, the sulfonium cation having a nitro group lowers the energy level of the lowest unoccupied molecular orbital (LUMO) of the frontier orbital theory due to the electron withdrawing effect of the nitro group, so that the cation is more likely to accept the generated secondary electrons, whereby the decomposition of the cation is promoted and the acid is effectively generated. Further, since the nitro group has a resonance structure in which the nitrogen atom is positively charged and one oxygen atom is negatively charged, the nitro group interacts with a proton of the generated acid at a negative charge on the oxygen atom, and a function as an acid diffusion inhibiting group can also be expected. By being charged as described above, an affinity with an alkaline developer is also high, and the residue after cationic decomposition is efficiently removed with respect to the alkaline developer, so that the risk of development defects in the exposed region is also reduced. In addition, iodine has an appropriate dissolution inhibitory ability and is thus effective for restraining the pattern in the unexposed region from collapse in alkaline developer. In the embodiment wherein the anion has an acid labile group, deprotection reaction of the acid labile group by the generated acid takes place to bring about a change of solubility, from which an enhancement of contrast and a higher sensitivity are expectable. In the exposed region of the positive resist film, as the affinity to alkaline developer is higher, the risk of development defects is lowered. The onium salt having an aromatic carboxylic acid anion has a relatively strong basicity whereby the strong acid generated from the photoacid generator is effectively trapped. Due to the synergy of these effects, a chemically amplified resist composition comprising the inventive onium salt exhibits a high dissolution contrast and can form line patterns with reduced LWR and hole patterns with improved CDU, both the patterns having collapse resistance. The inventive onium salt is thus useful as one component of a chemically amplified resist composition, especially of positive tone.

The inventive onium salt is advantageously used as a quencher. As used herein, the quencher refers to a compound capable of trapping the strong acid generated by a photoacid generator to prevent the acid from diffusing to the unexposed region and to assist in forming the desired pattern. As used herein, the photoacid generator refers to a compound capable of generating a strong acid upon exposure to high-energy radiation, and the strong acid refers to a compound having a sufficient acidity to induce deprotection reaction of an acid labile group. Since the aromatic carboxylic acid does not have an enough acidity to induce deprotection reaction of an acid labile group which is tertiary ester or tertiary ether, it is effective to separately add a photoacid generator capable of generating a strong acid, i.e., α-fluorinated sulfonic acid, imide acid or methide acid, for the purpose of inducing deprotection reaction of an acid labile group, as will be described later. It is noted that the photoacid generator capable of generating an α-fluorinated sulfonic acid, imide acid or methide acid may be of addition type or of polymer-bound type wherein the photoacid generator is bound to a base polymer.

In a system where the inventive onium salt capable of generating an aromatic carboxylic acid and a photoacid generator capable of generating an ultra-strong acid or perfluoroalkylsulfonic acid are co-present, the aromatic carboxylic acid and the perfluoroalkylsulfonic acid generate upon light exposure. Since the photoacid generator is not decomposed in its entirety, some photoacid generator remains undecomposed nearby. If the onium salt capable of generating an aromatic carboxylic acid and perfluoroalkylsulfonic acid are co-present at this point of time, first an ion exchange occurs between the perfluoroalkylsulfonic acid and the onium salt capable of generating an aromatic carboxylic acid whereby an onium salt of perfluoroalkylsulfonic acid is generated and the aromatic carboxylic acid is released. This is because the perfluoroalkylsulfonic acid salt having a high acid strength is more stable. On the other hand, where the perfluoroalkylsulfonic acid onium salt and the aromatic carboxylic acid are co-present, no ion exchange occurs. Similar ion exchange takes place not only with the perfluoroalkylsulfonic acid, but also with an arene sulfonic acid, alkyl sulfonic acid, imide acid or methide acid having a higher acid strength than the aromatic carboxylic acid generated by the inventive onium salt.

### Chemically Amplified Resist Composition

### (A) Quencher

The chemically amplified resist compositions of the invention essentially comprises (A) a quencher in the form of the onium salt having the formula (1).

In the chemically amplified resist composition, the amount of quencher (A) is preferably 0.1 to 40 parts by weight, more preferably 0.5 to 30 parts by weight per 80 parts by weight of a base polymer to be described just below. As long as the amount of quencher (A) is in the range, good sensitivity and resolution are achievable and the risk of foreign matter being formed after development or during stripping of resist film is avoided. The quencher (A) may be used alone or in combination of two or more.

(B) Base polymer

The chemically amplified resist composition may comprise a base polymer as component (B). The base polymer (B) preferably comprises repeat units having the formula (a1), which are also referred to as repeat units (a1).

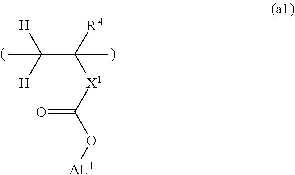

In the formula (a1), R^{A} is hydrogen, fluorine, methyl or trifluoromethyl.

In the formula (a1), X¹ is a single bond, phenylene group, naphthylene group, *—C(═O)—O—X¹¹— or *—C(═O)—NH—X¹¹—. The phenylene or naphthylene group may be substituted with an optionally fluorinated C₁-C₁₀ alkoxy moiety or halogen. X¹¹ is a C₁-C₁₀ saturated hydrocarbylene group which may contain a hydroxy moiety, ether bond, ester bond or lactone ring, or phenylene or naphthylene group. The asterisk (*) designates a point of attachment to the carbon atom in the backbone.

In the formula (a1), AL¹ is an acid labile group. Examples of the acid labile group include those described in JP-A 2013-080033 and JP-A 2013-083821.

Typical examples of the acid labile group include groups having the formulae (AL-3) to (AL-5):

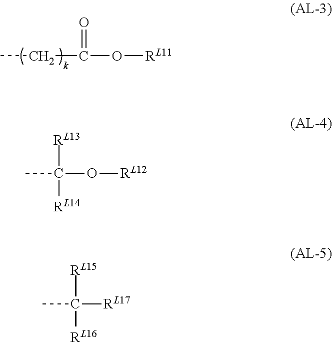

wherein the broken line represents a valence bond.

In the formulae (AL-3) and (AL-4), R^{L11} and R^{L12} are each independently a C₁-C₄₀ hydrocarbyl group which may contain a heteroatom such as oxygen, sulfur, nitrogen or fluorine. The hydrocarbyl group may be saturated or unsaturated and straight, branched or cyclic. The hydrocarbyl group is preferably a C₁-C₂₀ hydrocarbyl group.

In the formula (AL-3), k is an integer of 0 to 10, preferably 1, 2, 3, 4, or 5.

In the formula (AL-4), R^{L13} and R^{L14} are each independently hydrogen or a C₁-C₂₀ hydrocarbyl group which may contain a heteroatom such as oxygen, sulfur, nitrogen or fluorine. The hydrocarbyl group may be saturated or unsaturated and straight, branched or cyclic. The hydrocarbyl group is preferably a C₁-C₂₀ hydrocarbyl group. Any two of R^{L12}, R^{L13}, and R^{L14} may bond together to form a C₃-C₂₀ ring with the carbon atom or carbon and oxygen atoms to which they are attached. The ring is preferably a C₄-C₁₆ ring, particularly preferably an alicyclic ring.

In the formula (AL-5), R^{L15}, R^{L16}, and R^{L17} are each independently a C₁-C₂₀ hydrocarbyl group which may contain a heteroatom such oxygen, sulfur, nitrogen or fluorine. The hydrocarbyl group may be saturated or unsaturated and straight, branched or cyclic. The hydrocarbyl group is preferably a C₁-C₂₀ hydrocarbyl group. Any two of R^{L15}, R^{L16} and R^{L17} may bond together to form a C₃-C₂₀ ring with the carbon atom to which they are attached. The ring is preferably a C₄-C₁6 ring, particularly preferably an alicyclic ring.

Examples of the repeat units (a1) include those shown below, but not limited thereto. In the formulae, R^{A} and AL¹ are as defined above.

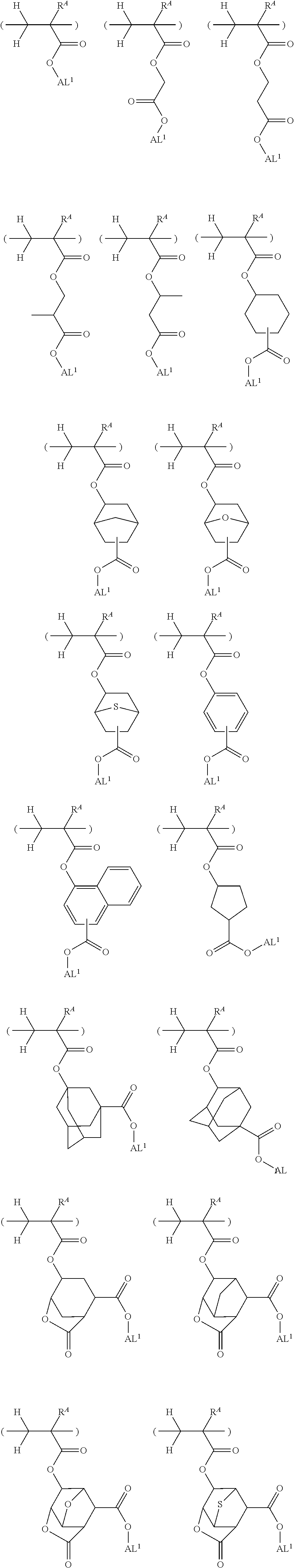

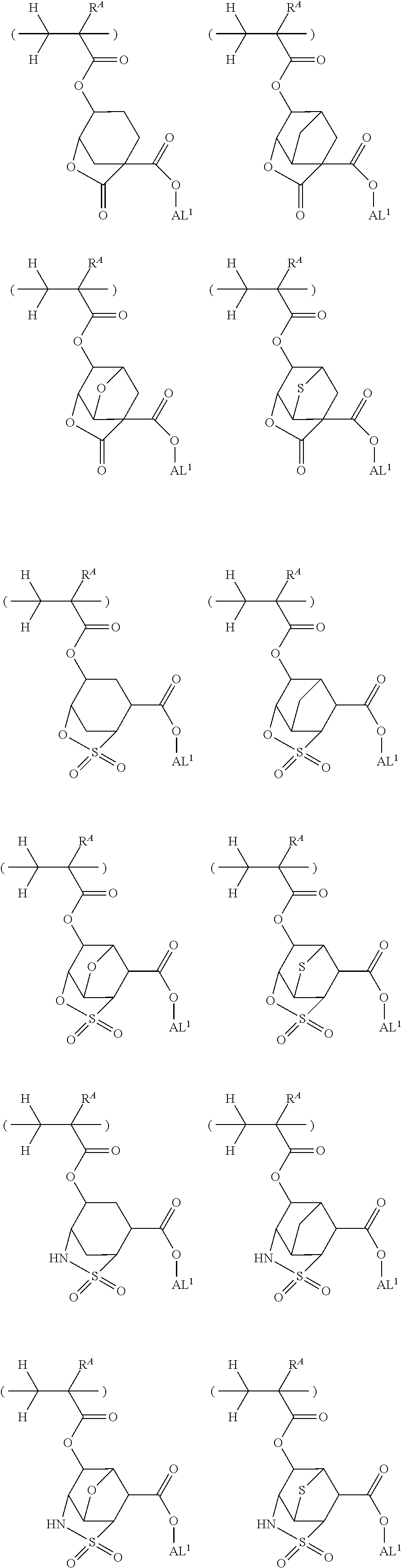

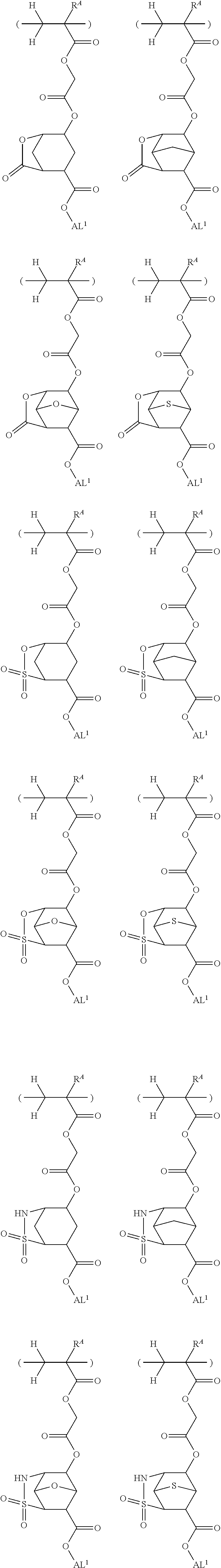

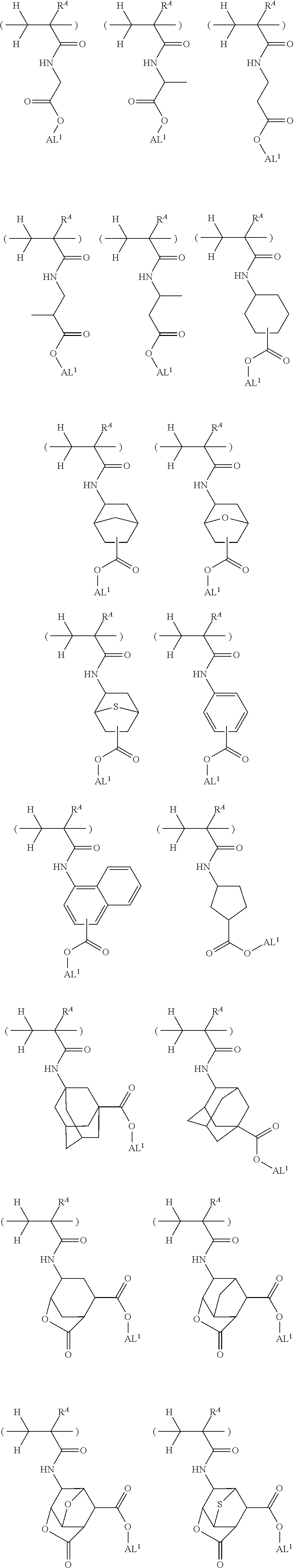

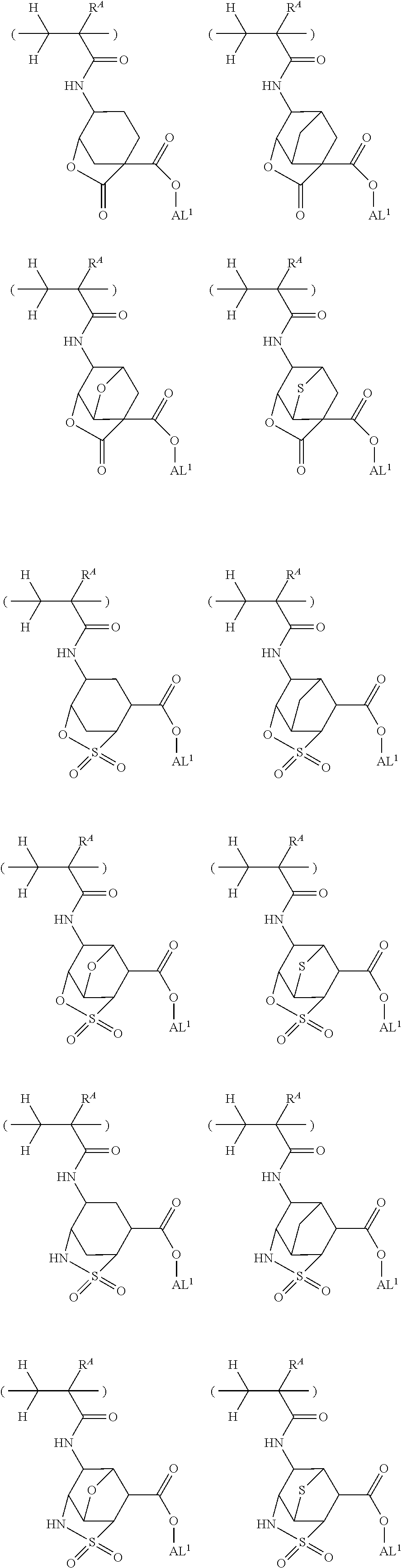

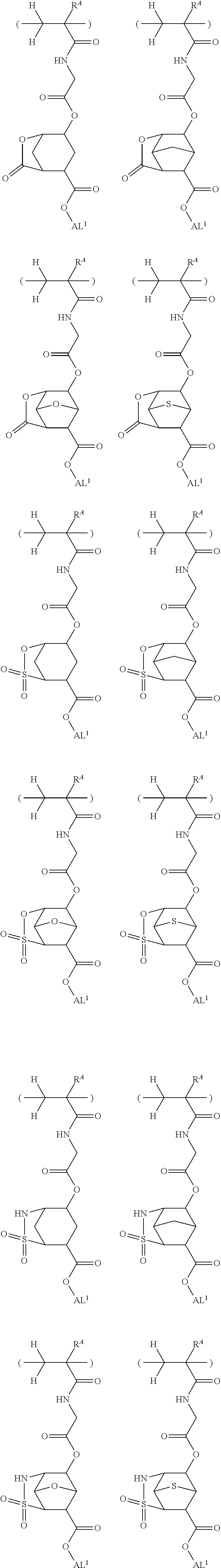

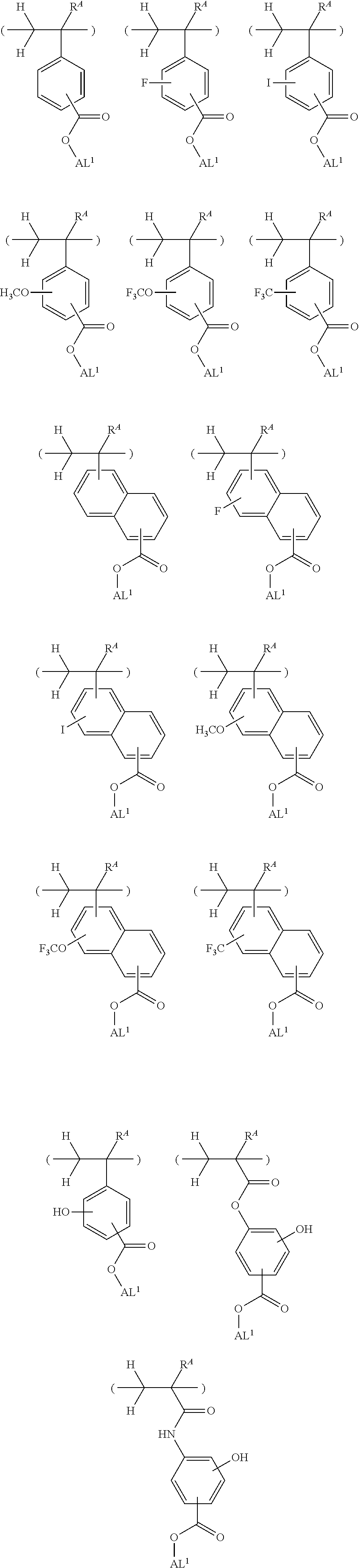

The base polymer may comprise repeat units having the formula (a2), which are also referred to as repeat units (a2).

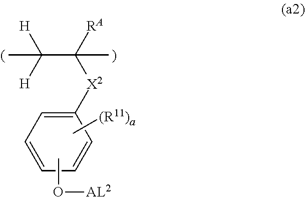

In the formula (a2), R^{A} is hydrogen, fluorine, methyl or trifluoromethyl. X2 is a single bond or *—C(═O)—O—. The asterisk (*) designates a point of attachment to the carbon atom in the backbone. R¹¹ is halogen, cyano, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. AL² is an acid labile group. Examples of the acid labile group include the acid labile groups exemplified above for AL¹. a is 0, 1, 2, 3, or 4, preferably 0 or 1.

Examples of the repeat units (a2) include those shown below, but not limited thereto. In the formulae, R^{A} and AL² are as defined above.

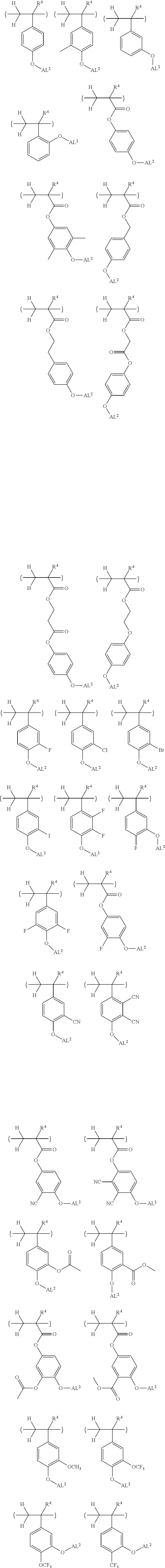

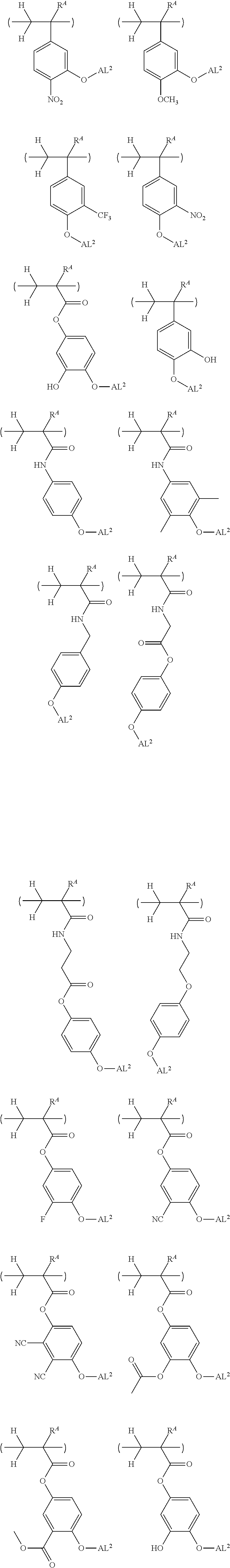

The base polymer may comprise repeat units having the formula (a3), which are also referred to as repeat units (a3).

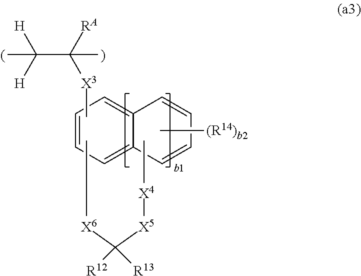

In the formula (a3), b1 is 0 or 1. The relevant structure is a benzene ring in case of b1=0, and a naphthalene ring in case of b1=1. The benzene ring corresponding to b1=0 is preferred from the aspect of solvent solubility. b2 is 0, 1, 2, or 3 in case of b1=O, and is 0, 1, 2, 3, 4, or 5 in case of b1=1. b2 is preferably 0, 1, 2, or 3, more preferably 0, 1, or 2 from the aspect of availability of reactants.

In the formula (a3), R^{A} is hydrogen, fluorine, methyl or trifluoromethyl. R^{A} is preferably hydrogen or methyl, more preferably hydrogen.

In the formula (a3), X³ is a single bond, *—C(═O)—O—, or *—C(═O)—NH—. the asterisk (*) designates a point of attachment to the carbon atom in the backbone, Among them, X³ is preferably a single bond or *—C(═O)—O—, more preferably a single bond.

In the formula (a3), R¹² and R¹³ are each independently hydrogen or a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, allyl, propenyl, butenyl, and hexenyl; C₃-C₂₀ cyclic unsaturated hydrocarbyl groups such as cyclohexenyl; C₂-C₂₀ aryl groups such as phenyl and naphthyl; C₇-C₂₀ aralkyl groups such as benzyl, 1-phenylethyl, and 2-phenylethyl; and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

Also, R¹² and R¹³ may bond together to form a ring with the carbon atoms to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

In the formula (a3), R¹⁴ is halogen, hydroxy, cyano, nitro, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}). R^{14A} and R^{14B} are each independently hydrogen or a C₁-C₆ hydrocarbyl group. The halogen atom is preferably fluorine, chlorine, bromine or iodine, more preferably fluorine or iodine. The hydrocarbyl group and hydrocarbyl moiety in the hydrocarbyloxy, hydrocarbyloxycarbonyl and hydrocarbylthio groups may be saturated or unsaturated and straight, branched or cyclic. Examples thereof include those exemplified above for the hydrocarbyl groups represented by R¹² and R¹³. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. A plurality of R¹⁴ may be identical or different when b2 is 2 or more.

Also, when b2 is 2 or more, a plurality of R¹⁴ may bond together to form a ring with the carbon atom of the aromatic ring to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

In the formula (a3), X⁴ is a single bond, a C₁-C₄ aliphatic hydrocarbylene group, carbonyl group, sulfonyl group, or a group obtained by combining the foregoing. Of these, X⁴ is preferably a single bond, carbonyl group or sulfonyl group from the aspect of availability of reactants, more preferably a single bond or a carbonyl group from the aspect of a polar group created after reaction.

In the formula (a3), X⁵ and X⁶ are each independently oxygen or sulfur. Notably, X⁴ and X⁶ are bonded to vicinal carbon atoms on the aromatic ring. X⁵ and X⁶ may be identical or different. Both X⁵ and X⁶ are preferably oxygen from the aspect of reactivity.

Examples of the repeat units (a3) include those shown below, but not limited thereto. In the formulae, R^{A} is as defined above, and Me is methyl. The bond positions of substituents on the aromatic ring are interchangeable.

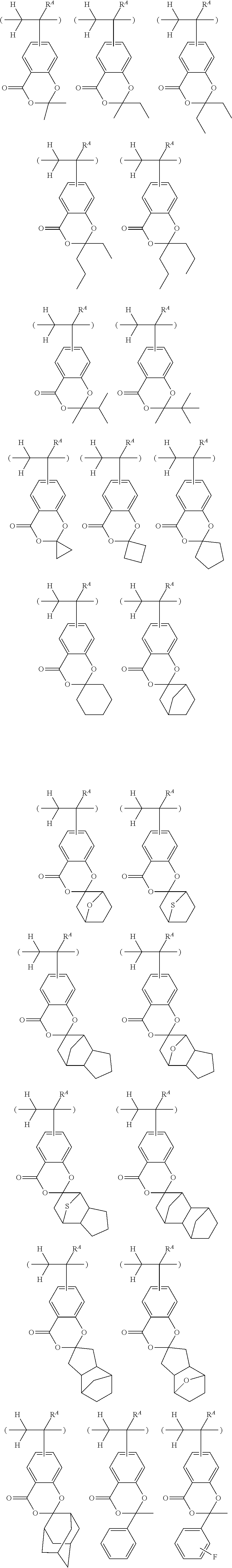

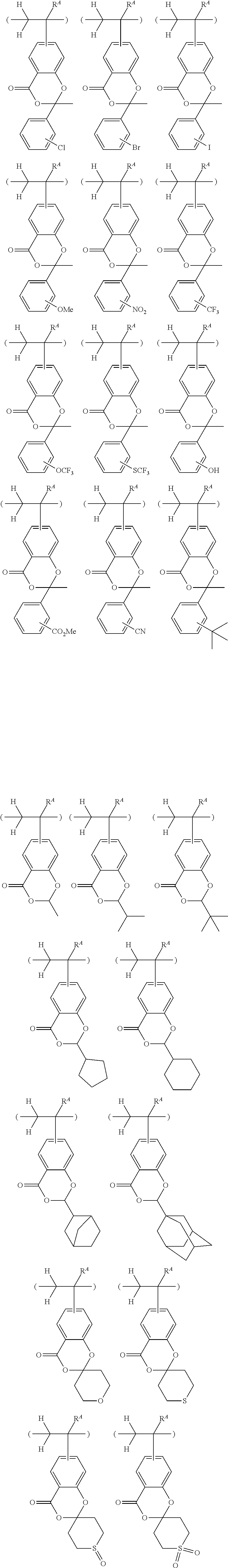

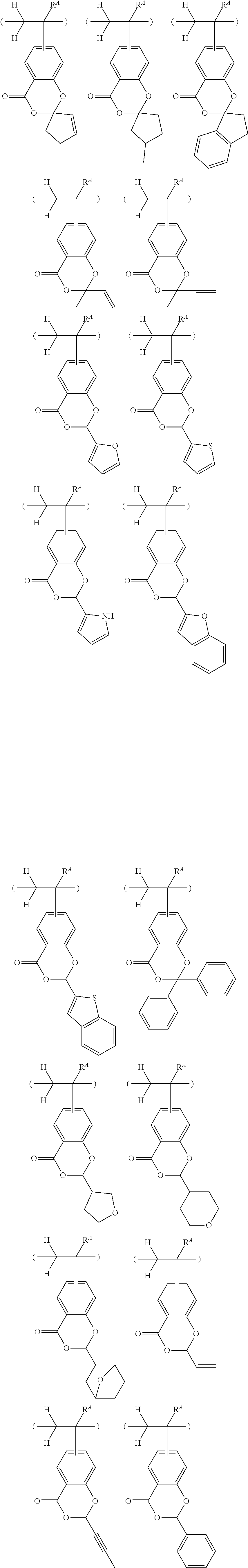

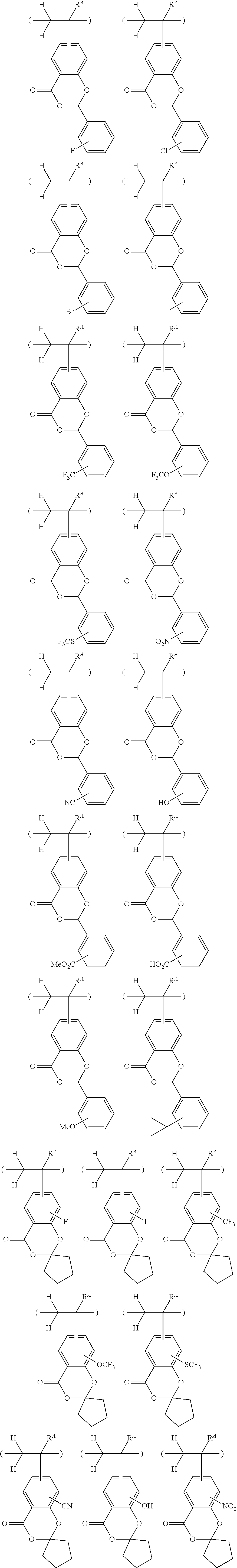

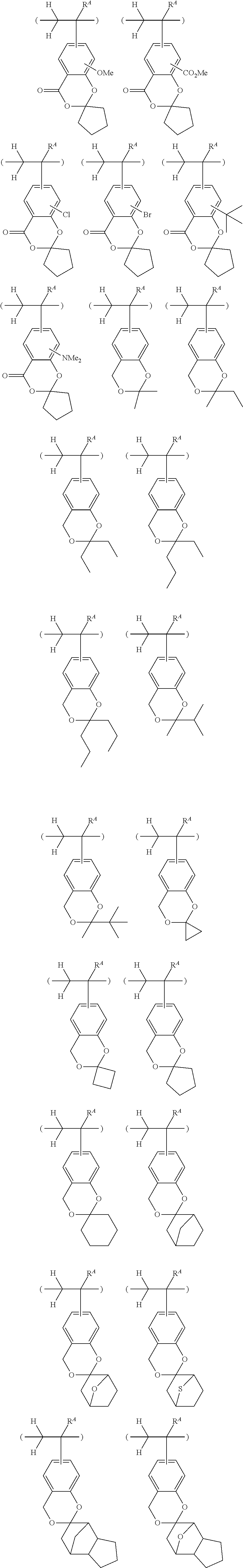

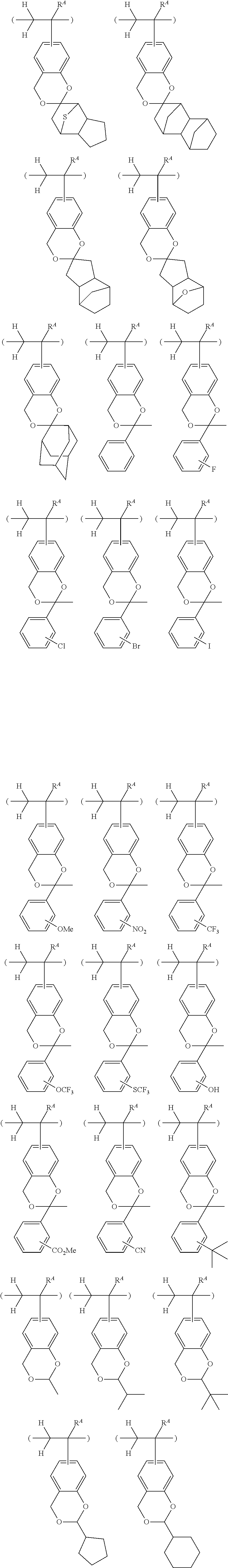

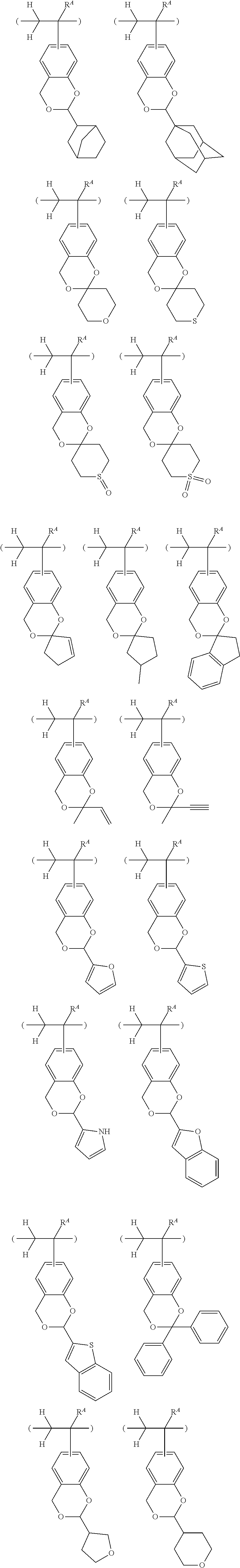

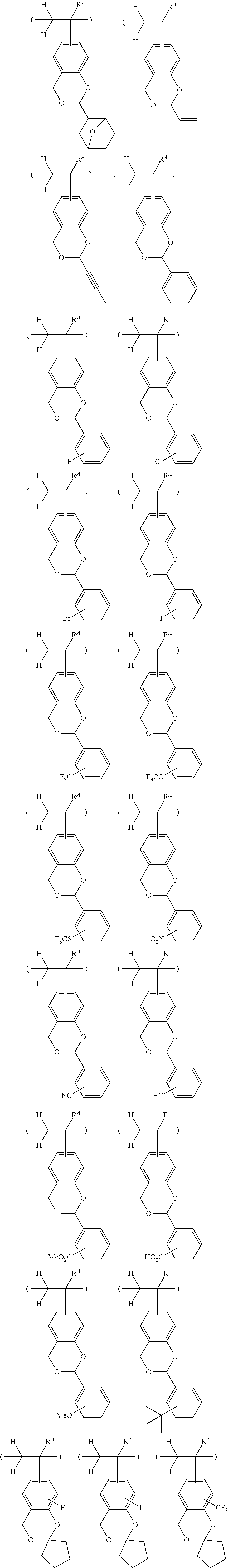

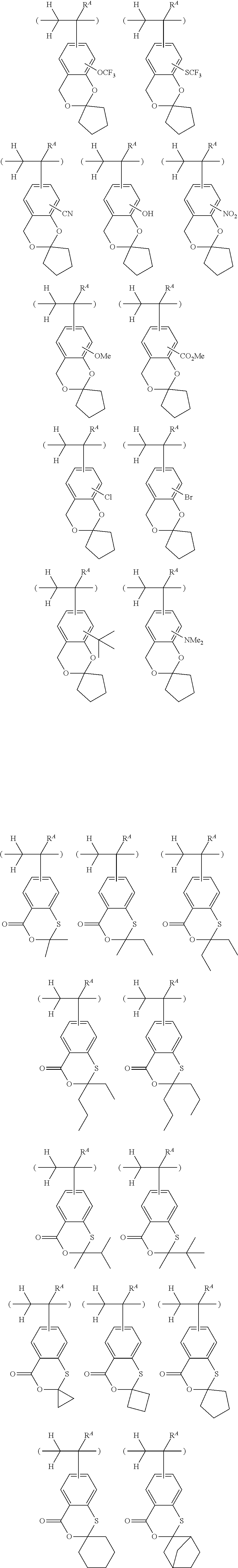

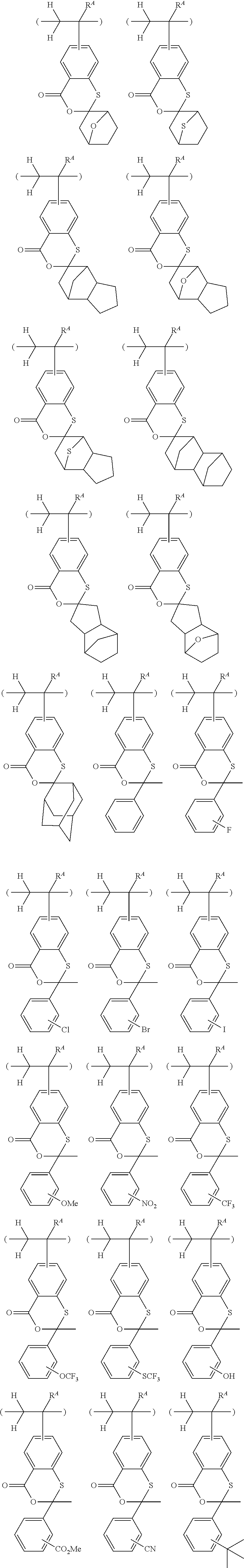

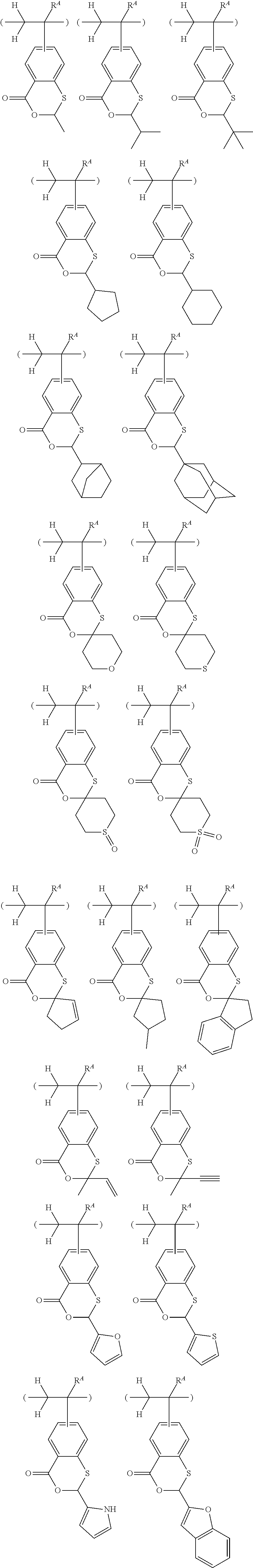

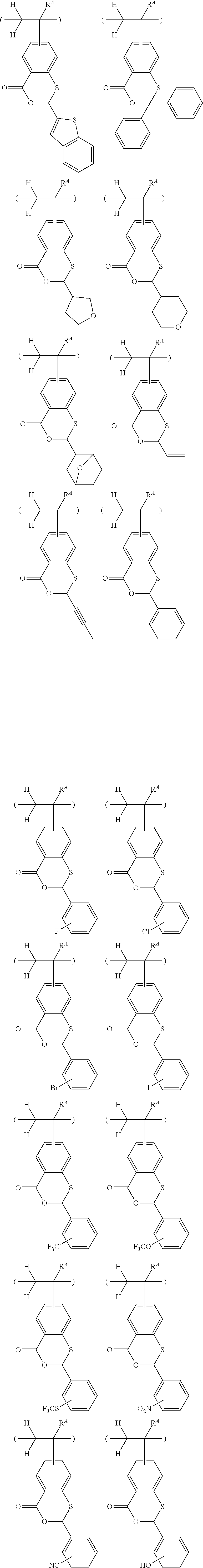

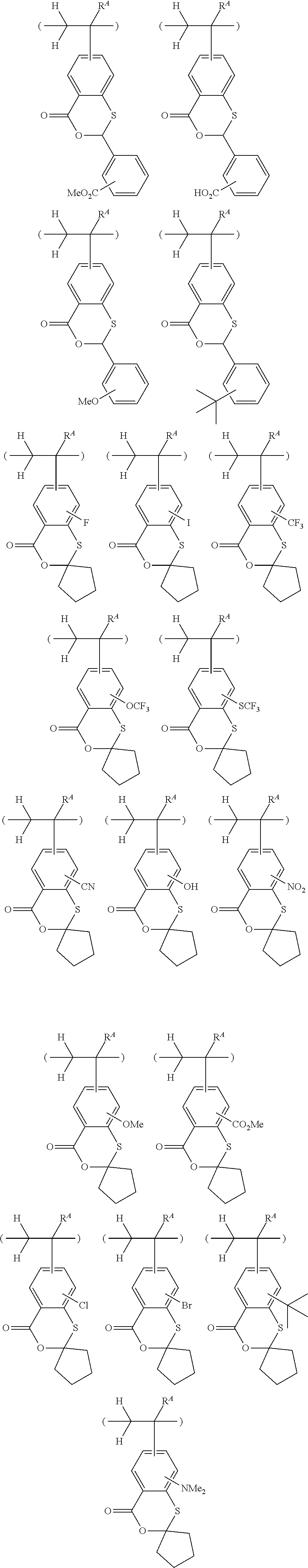

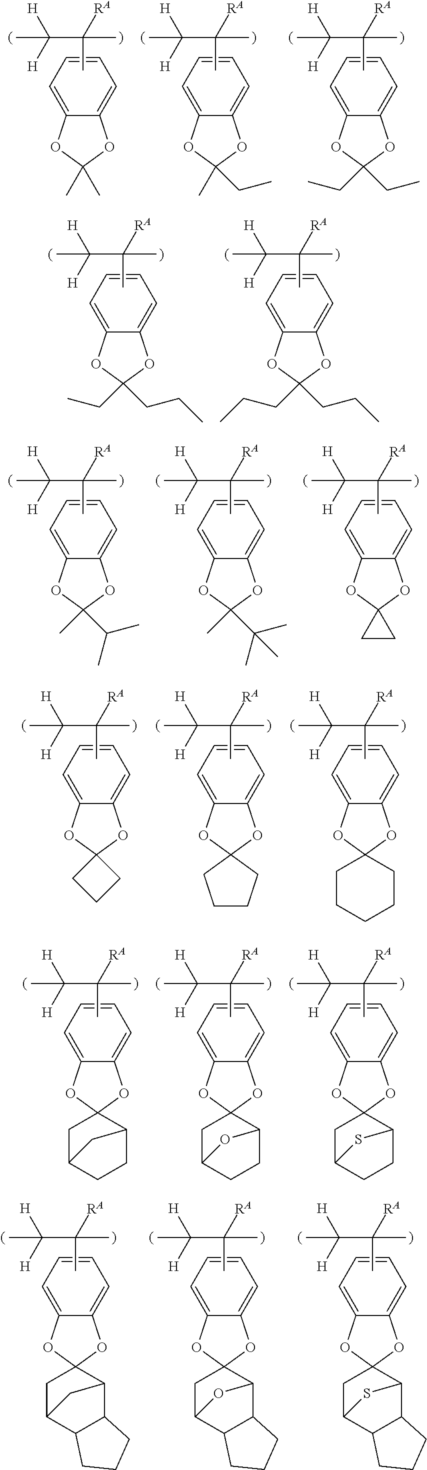

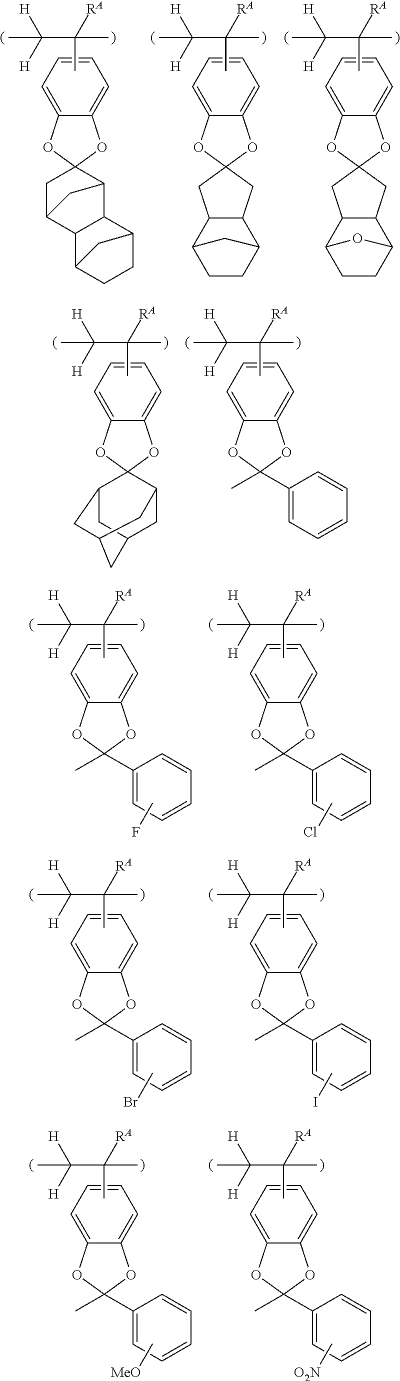

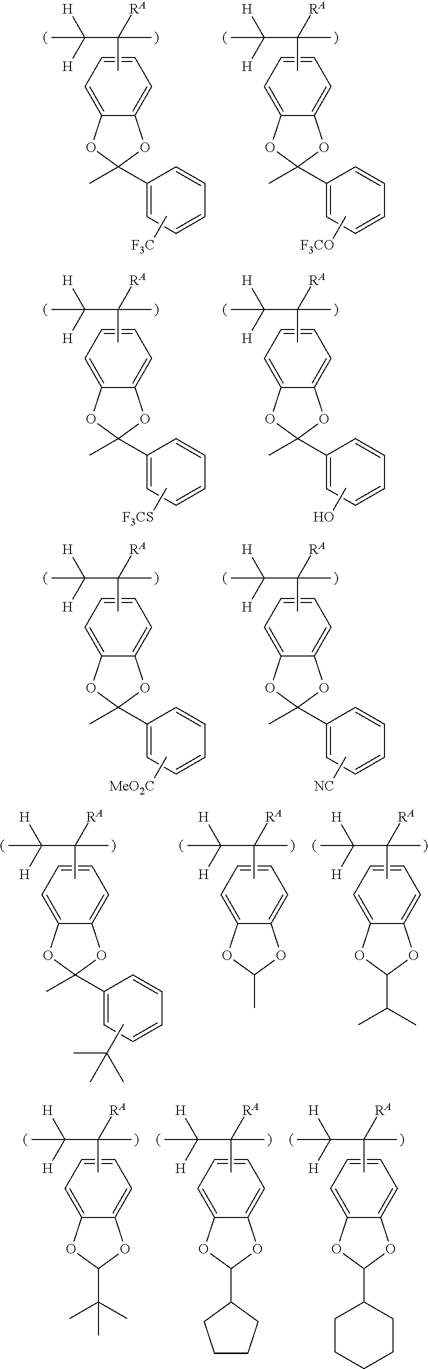

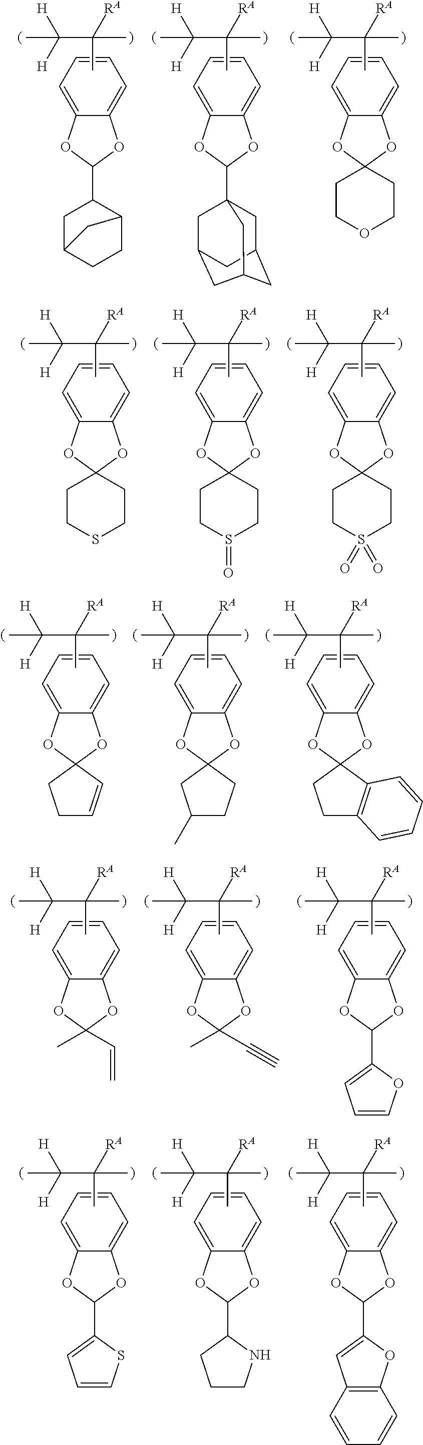

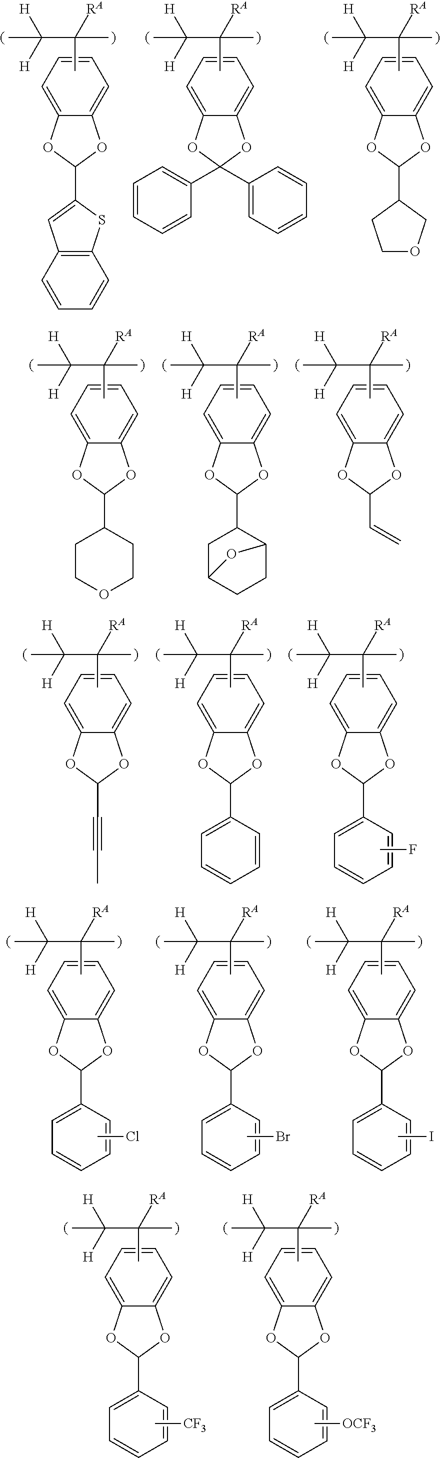

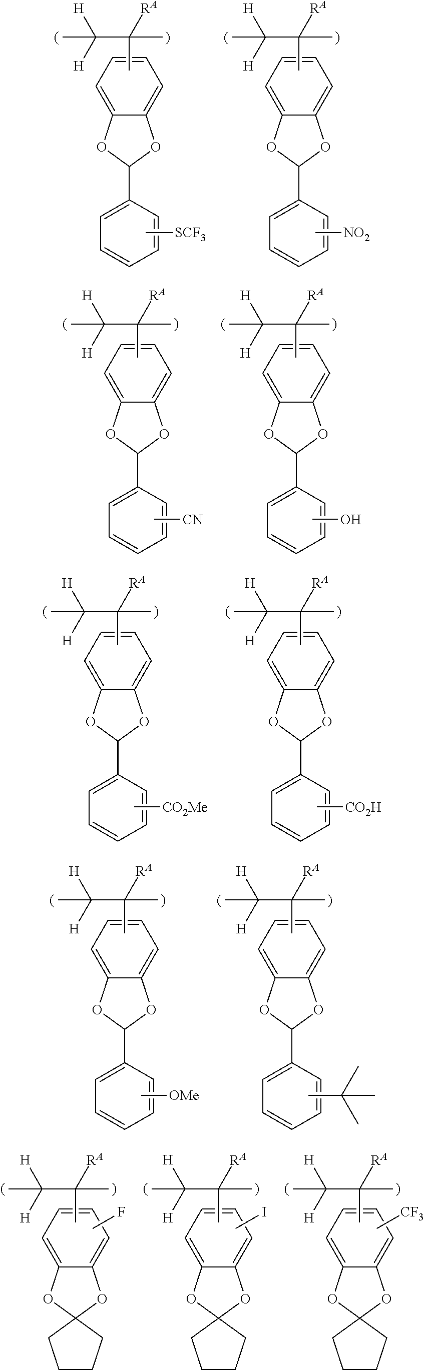

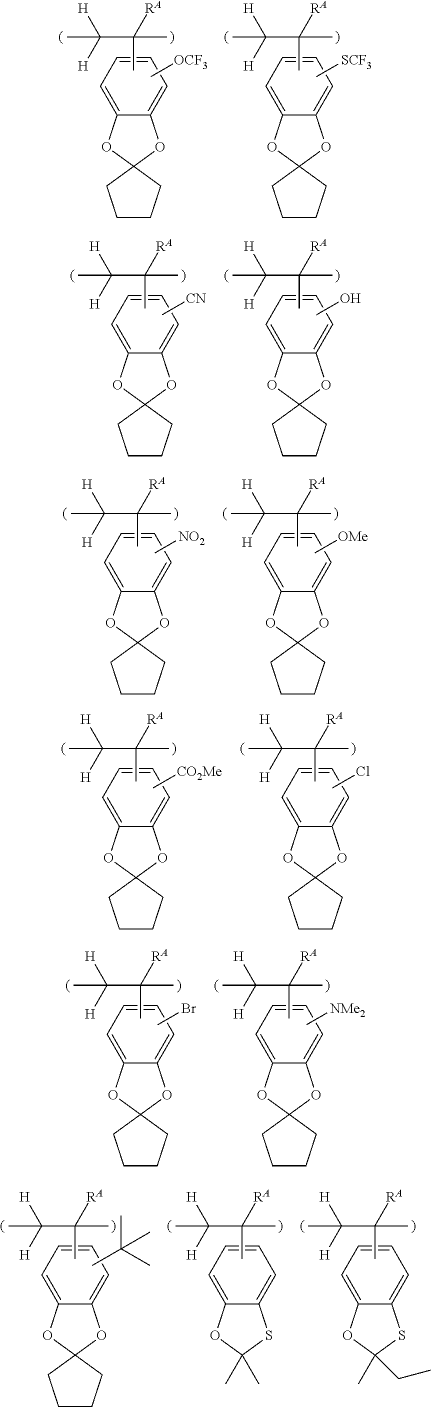

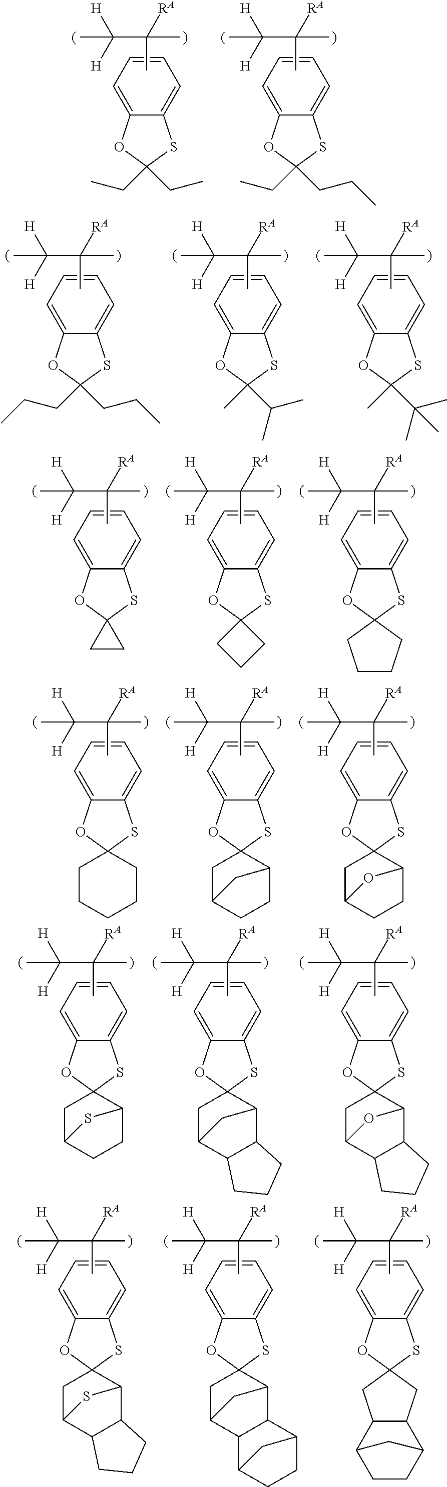

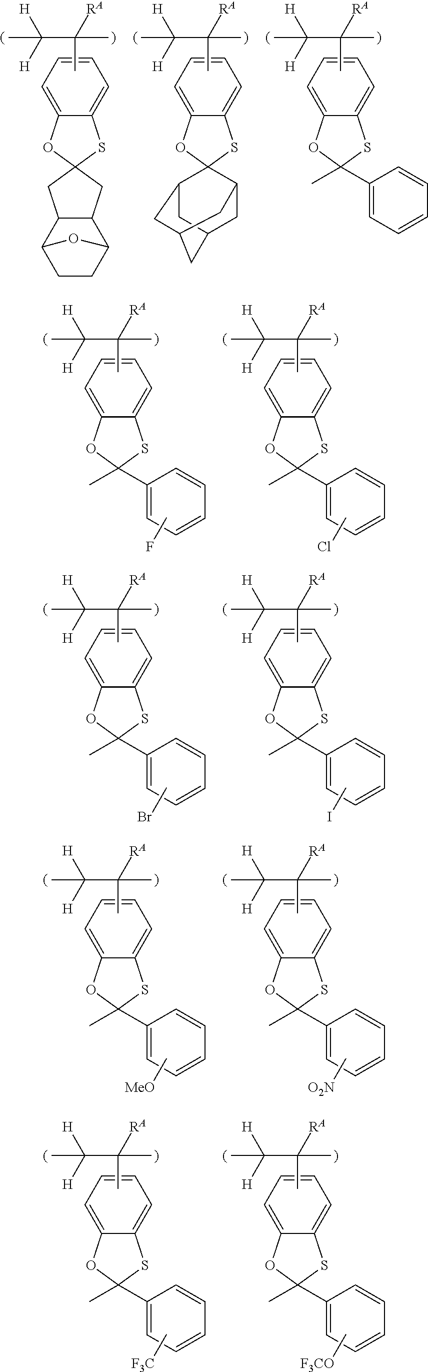

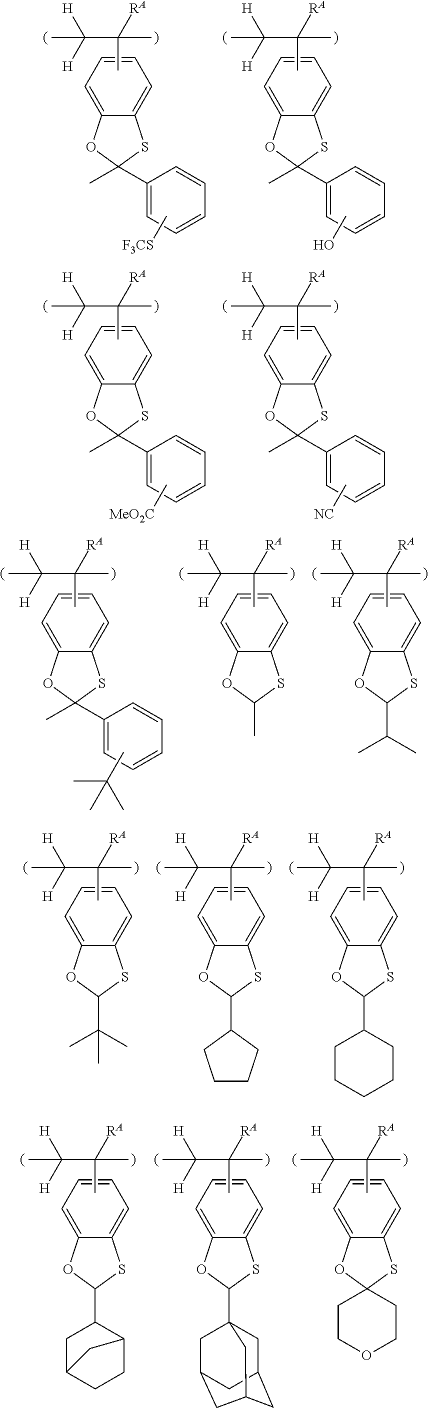

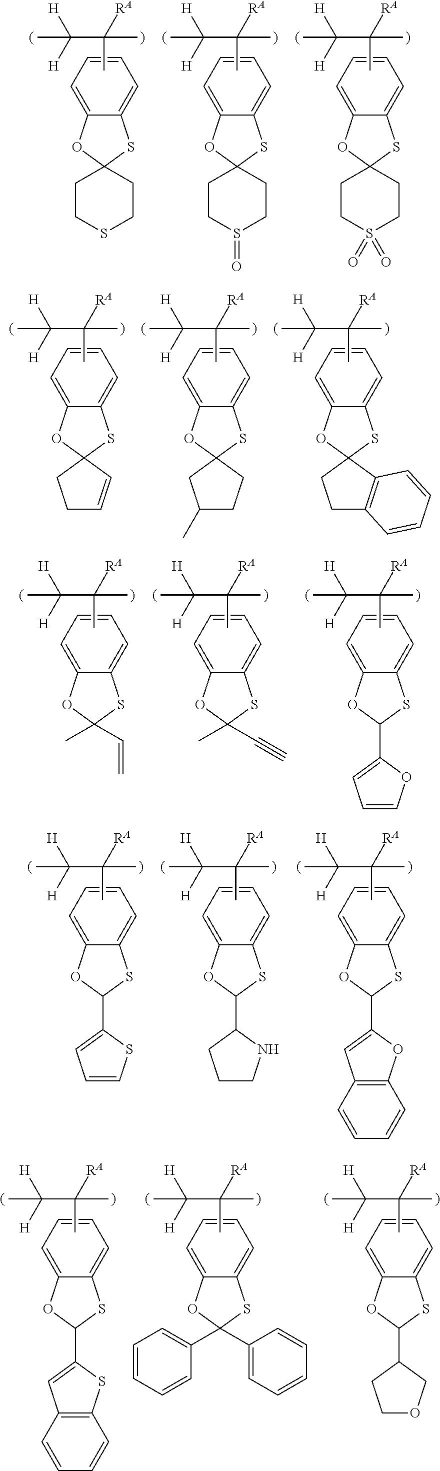

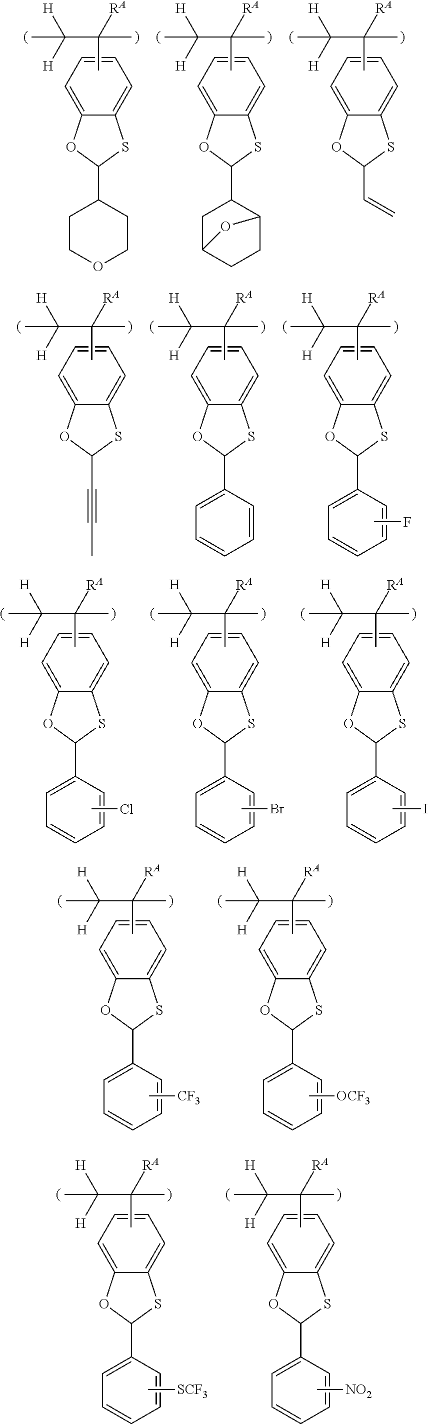

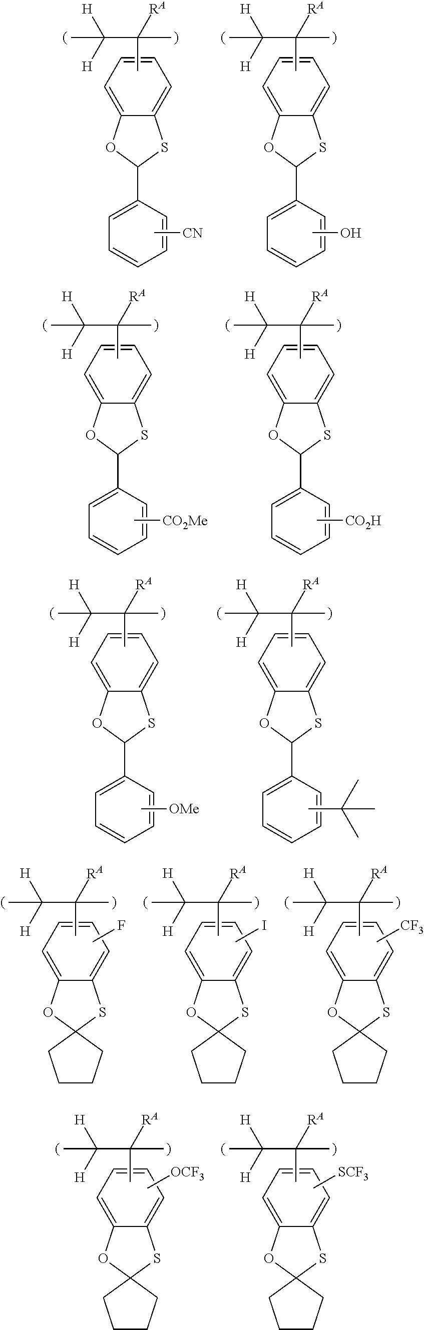

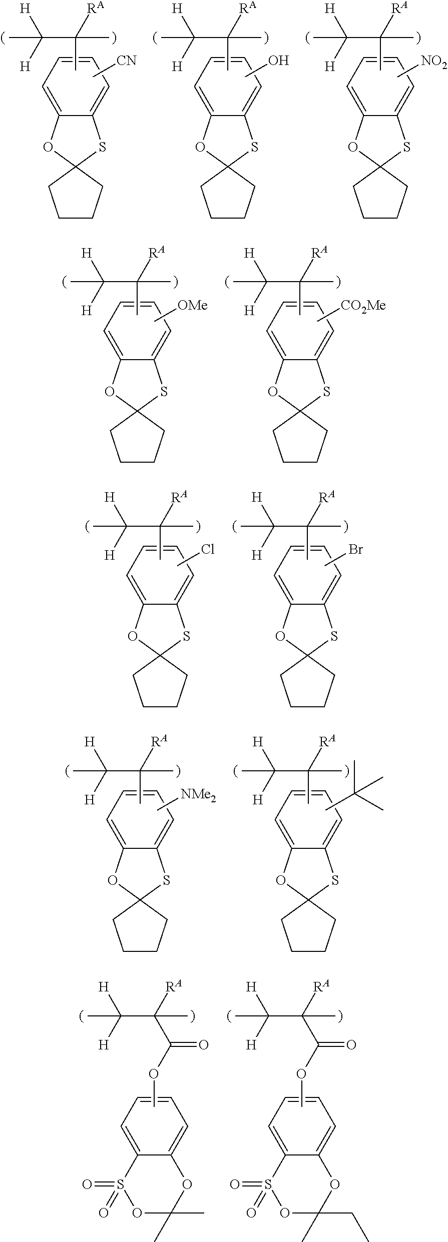

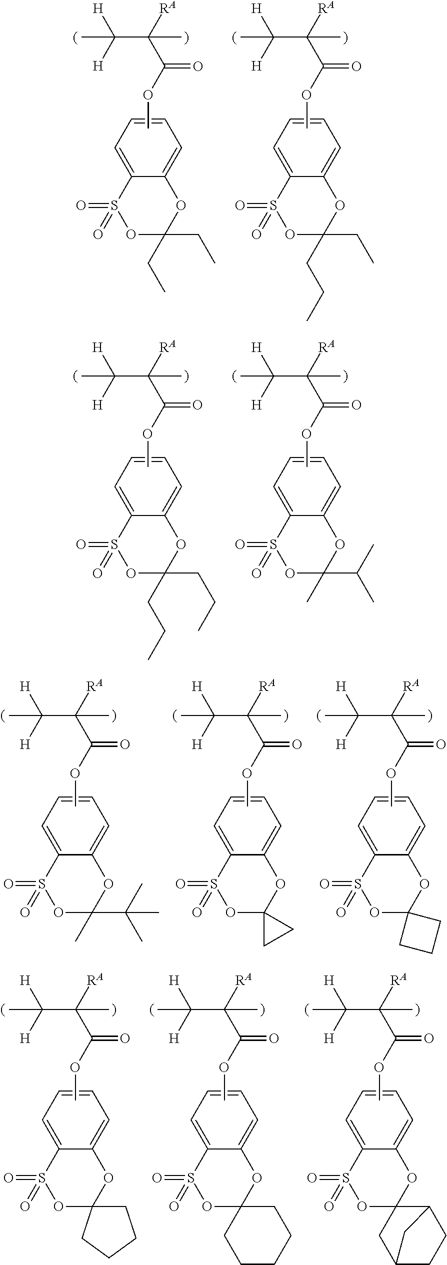

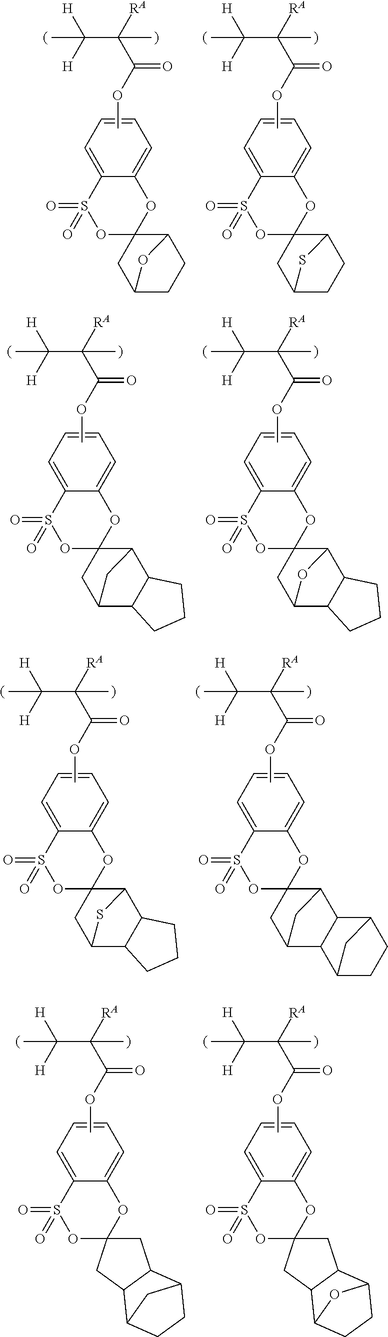

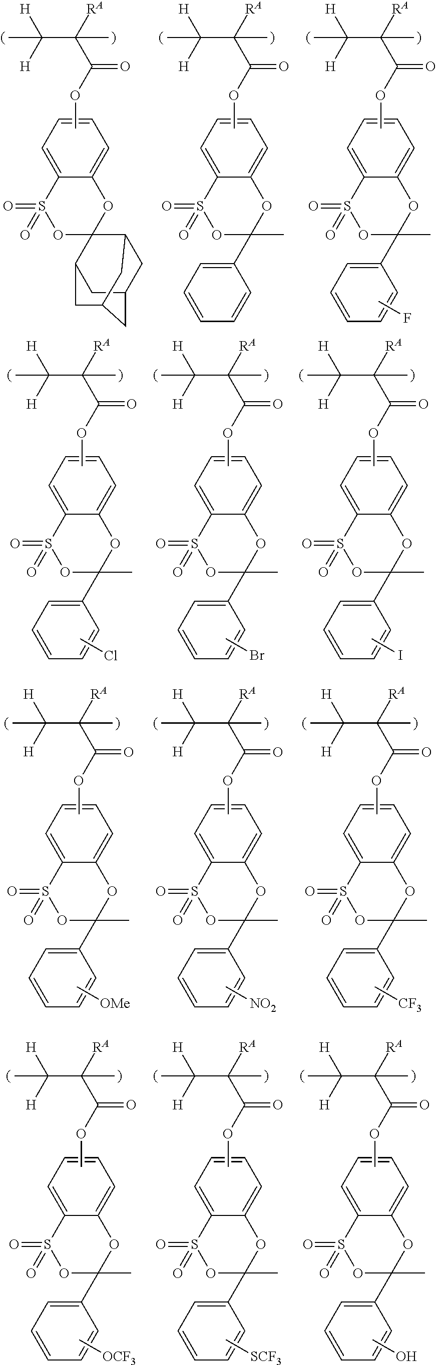

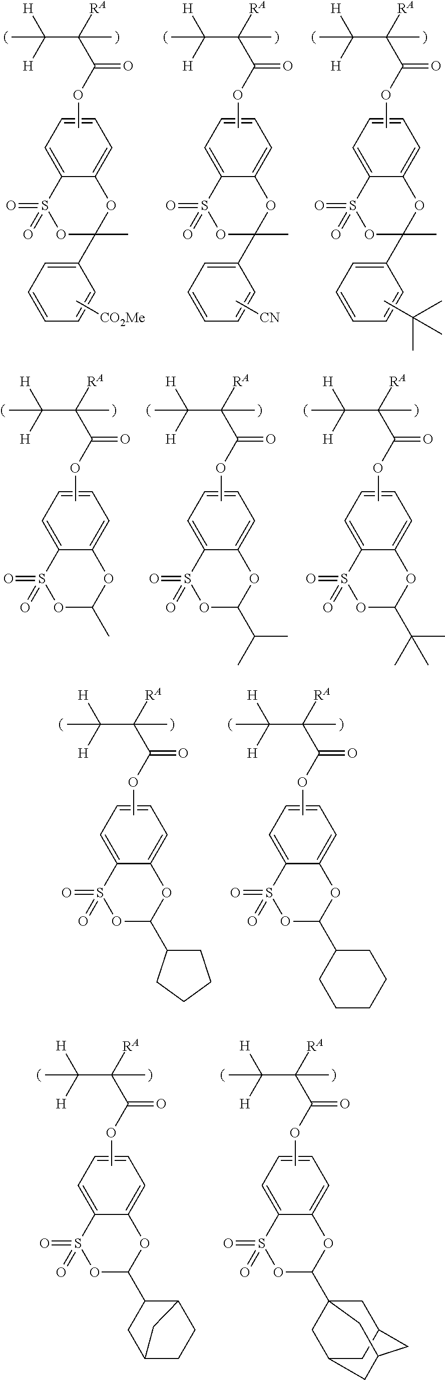

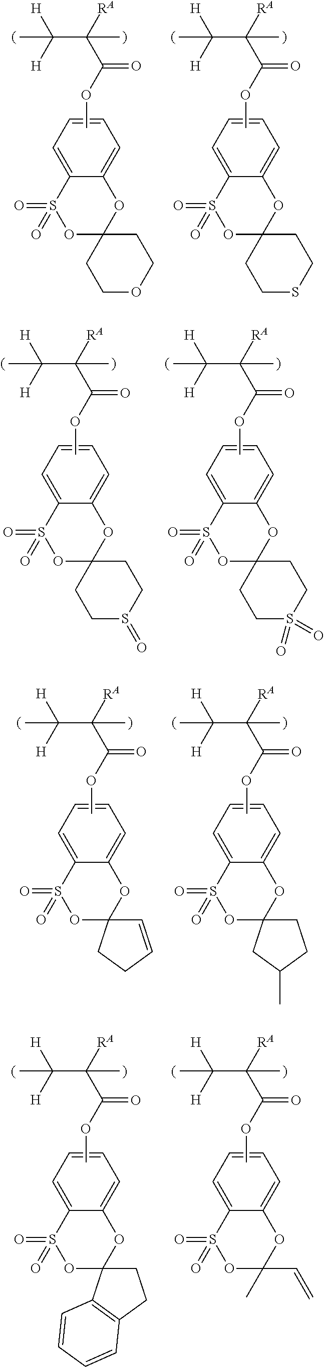

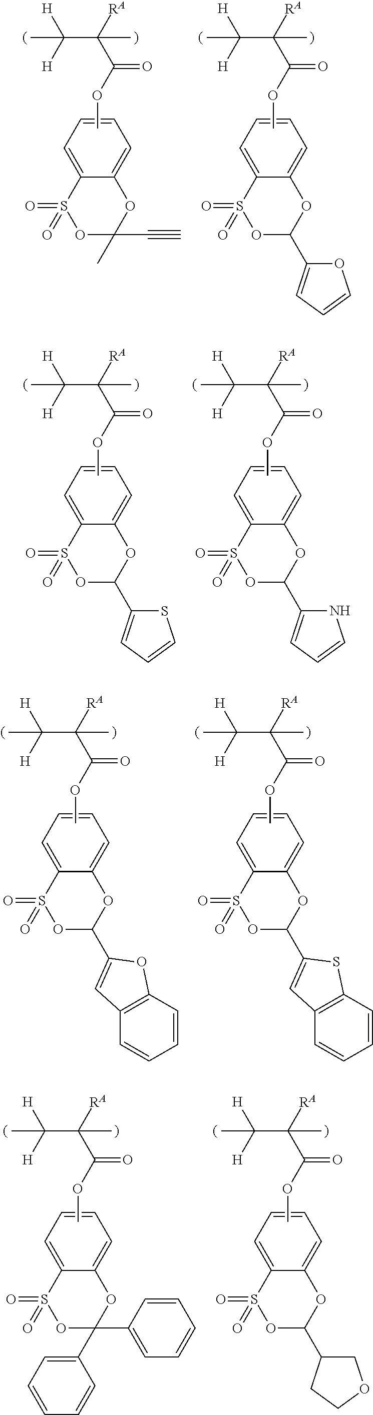

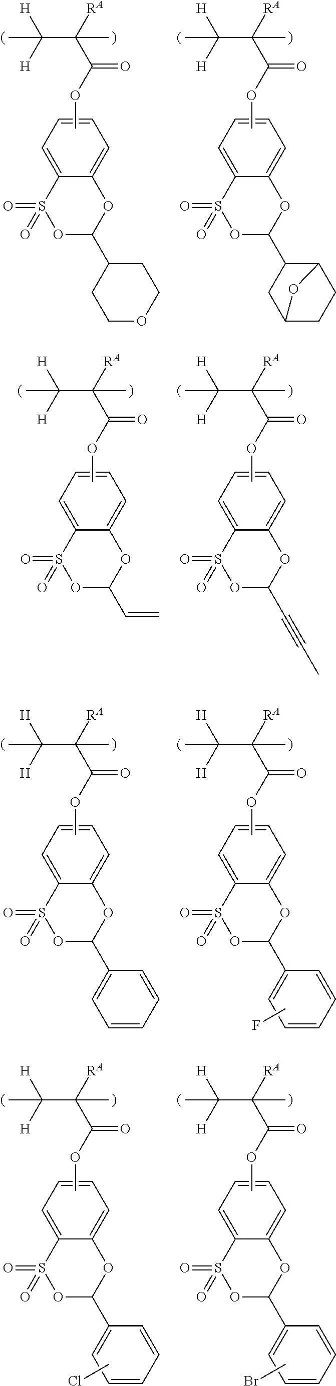

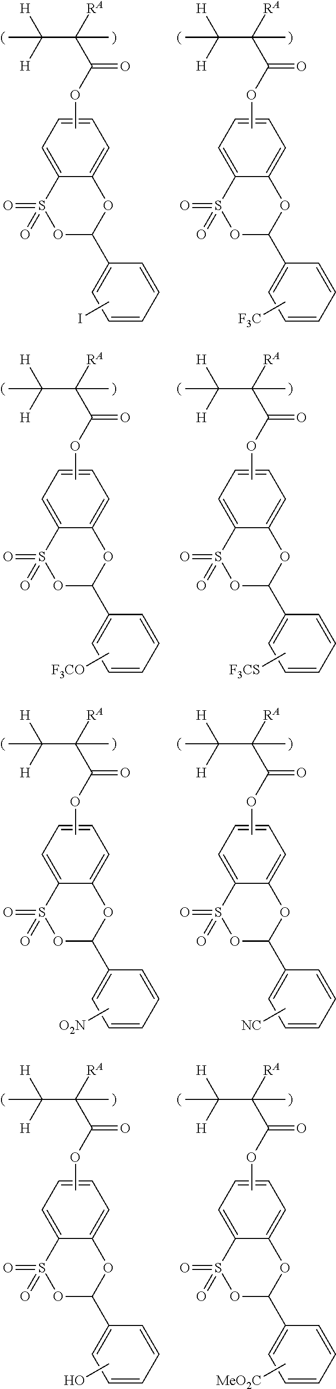

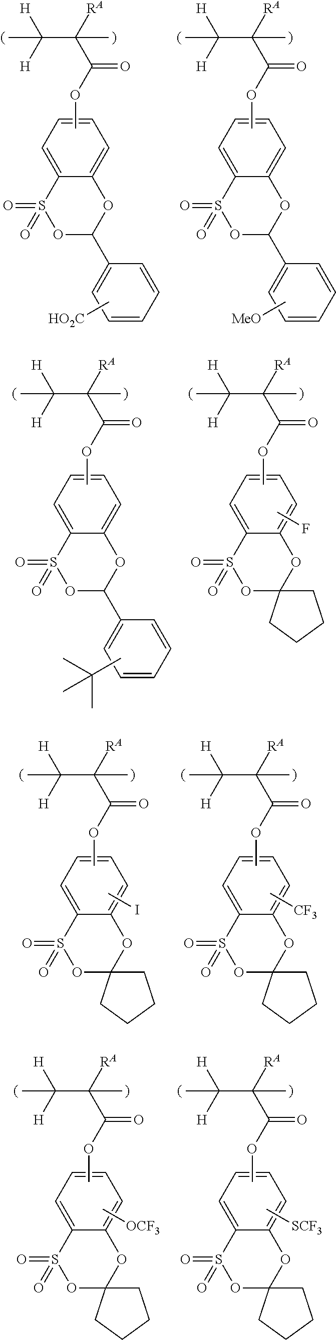

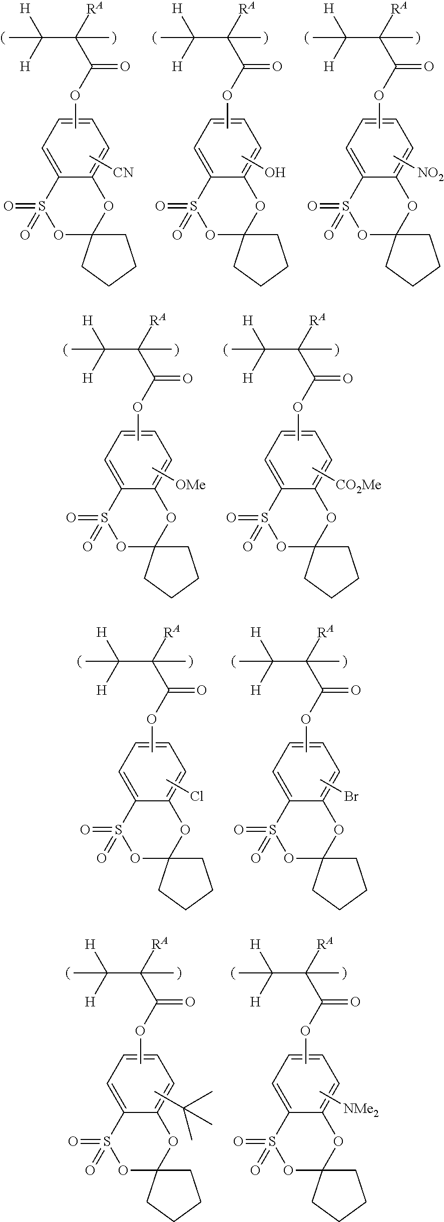

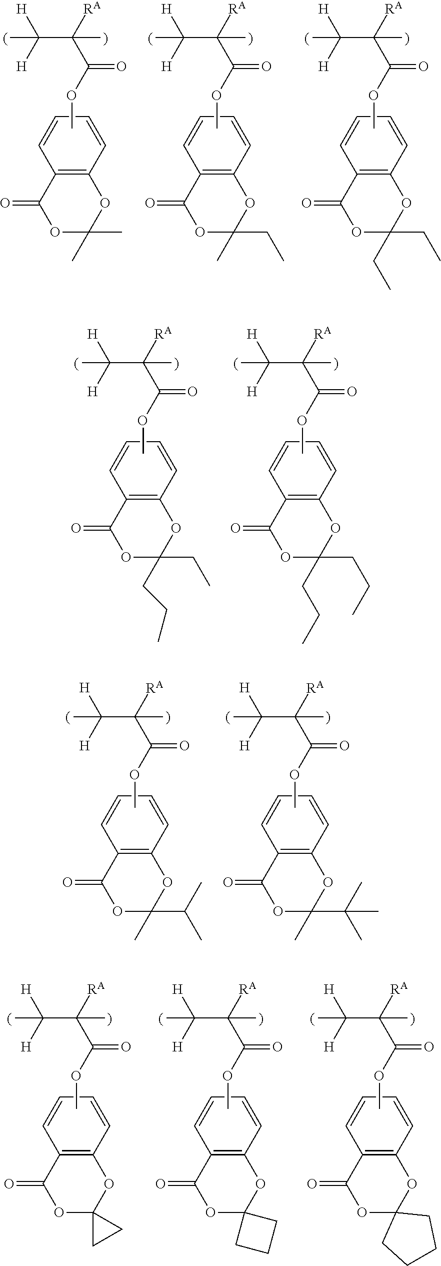

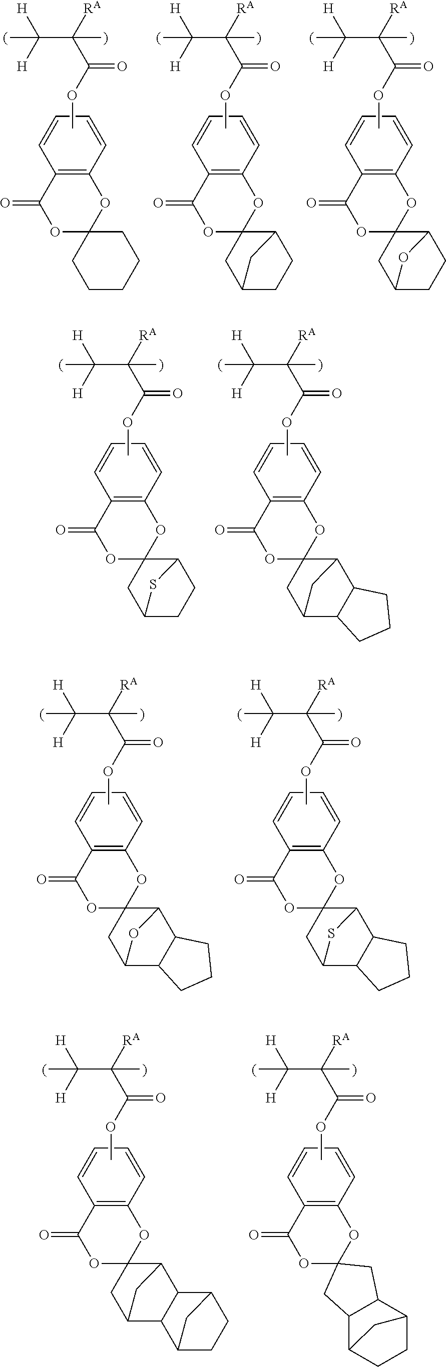

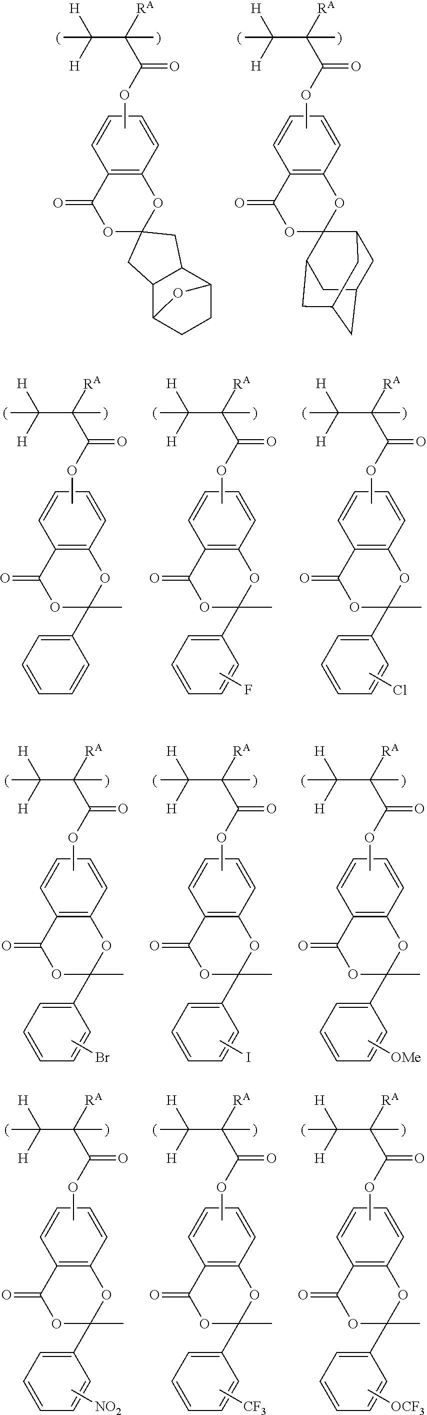

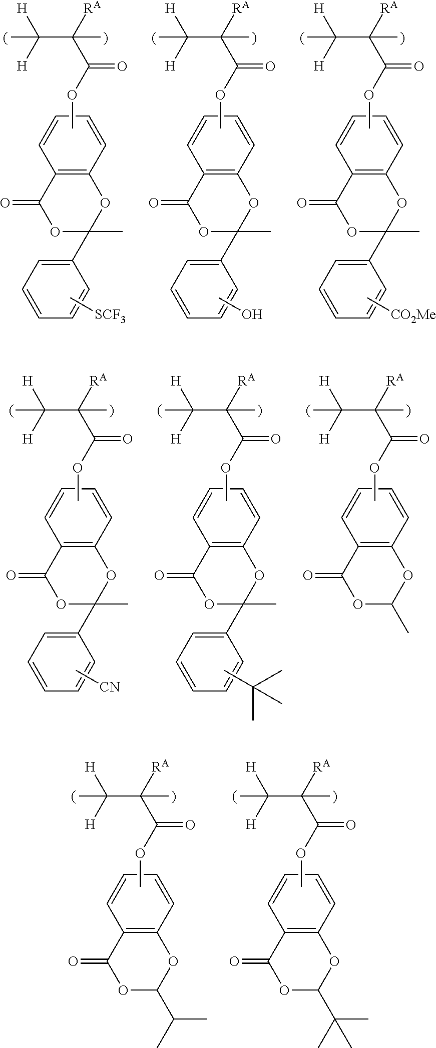

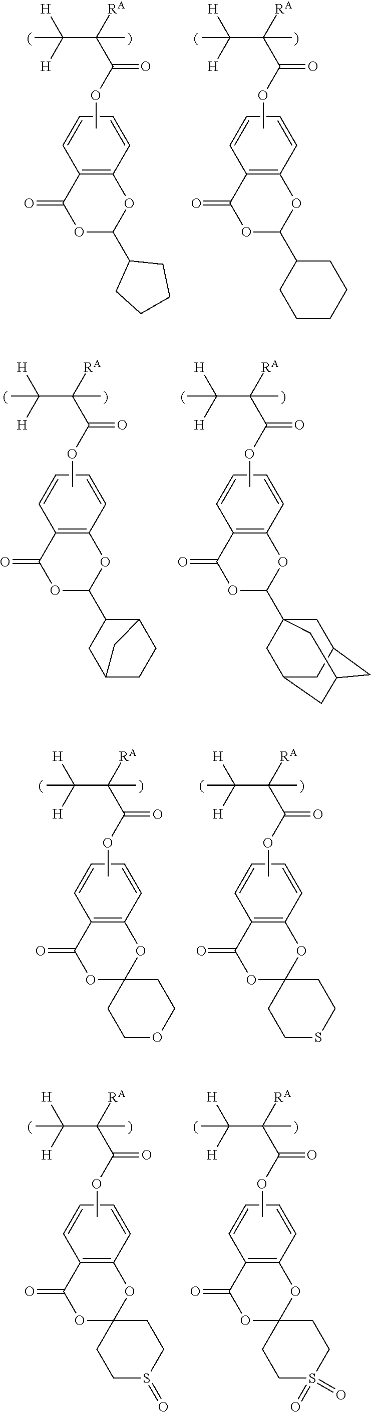

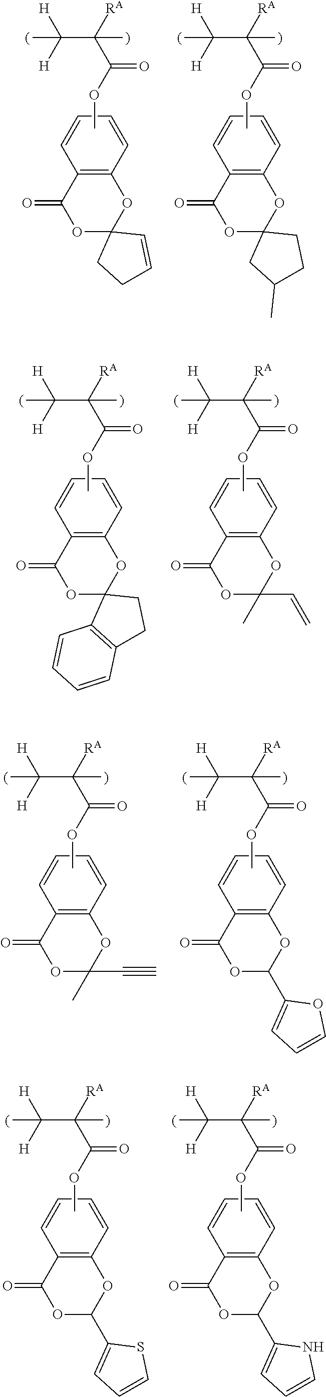

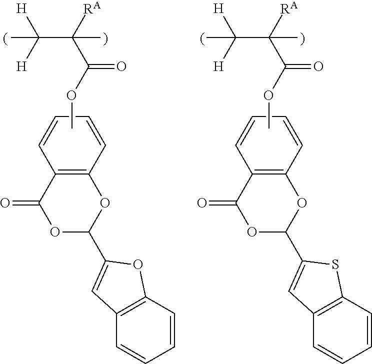

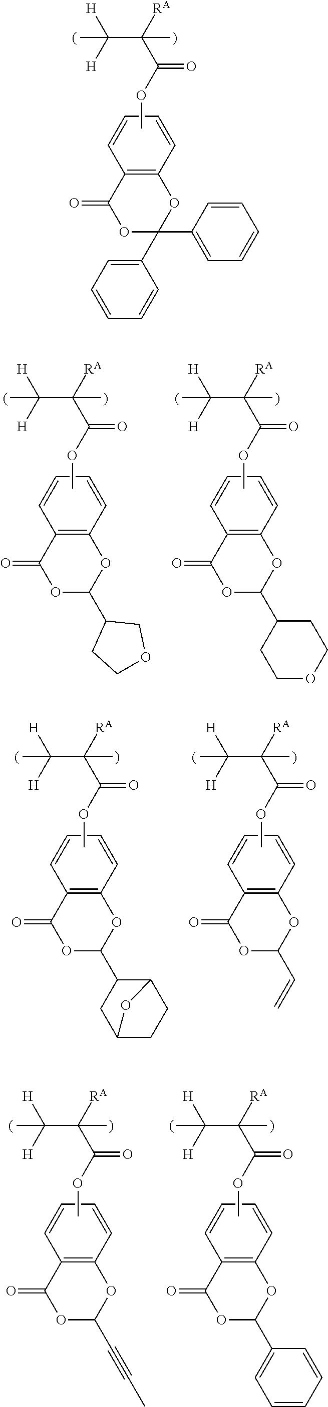

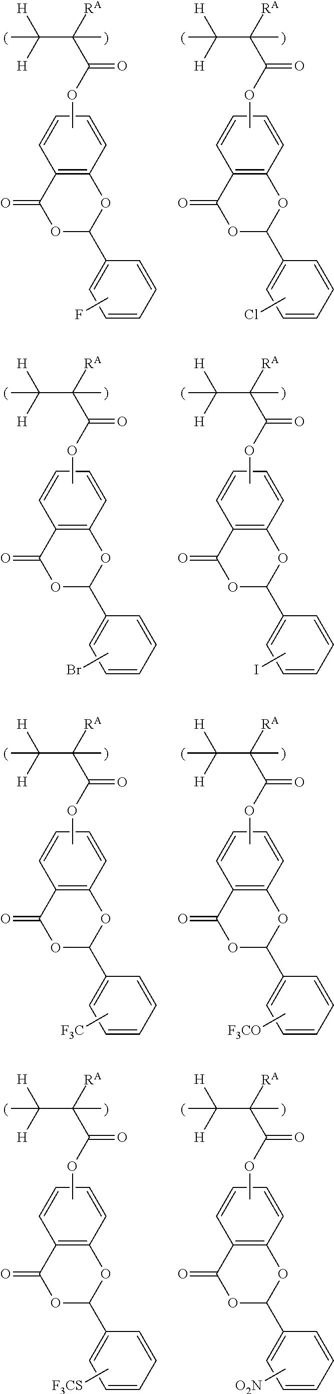

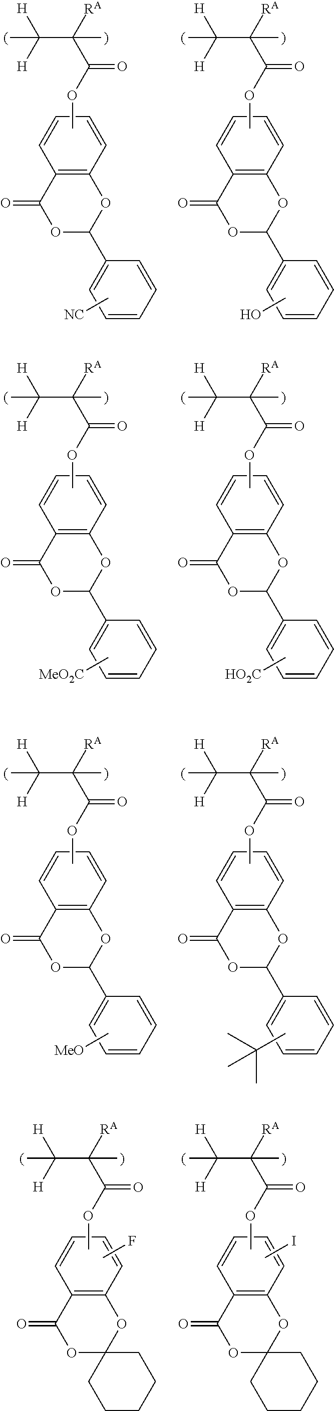

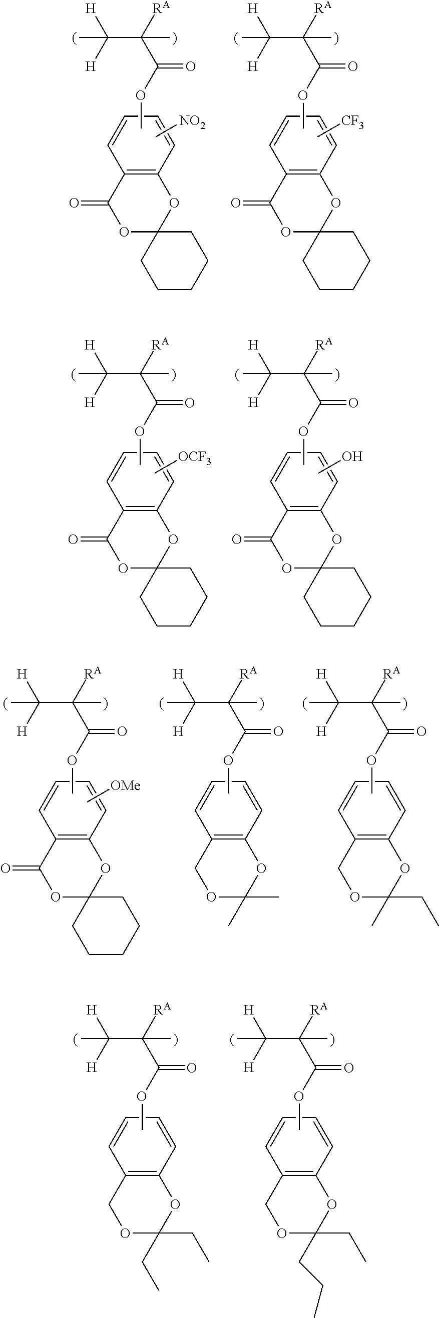

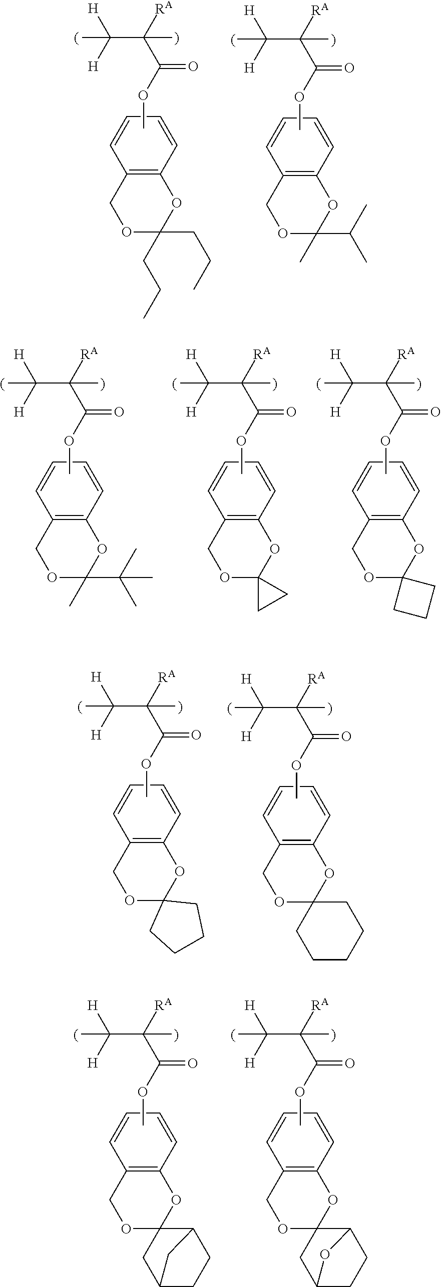

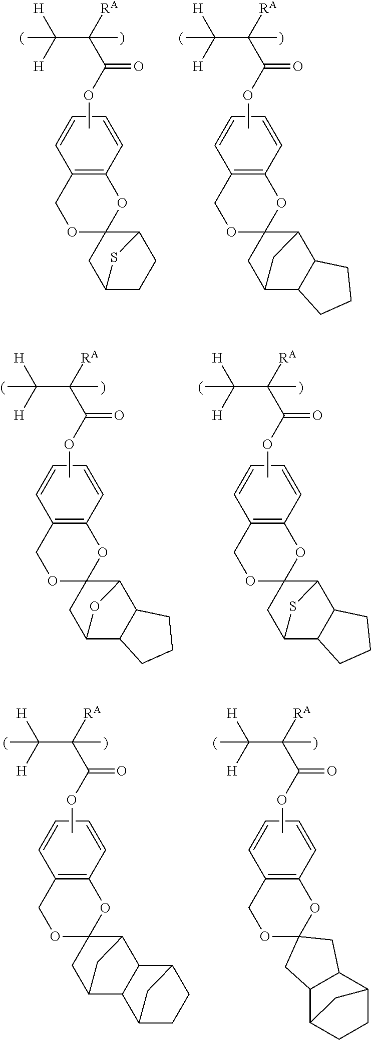

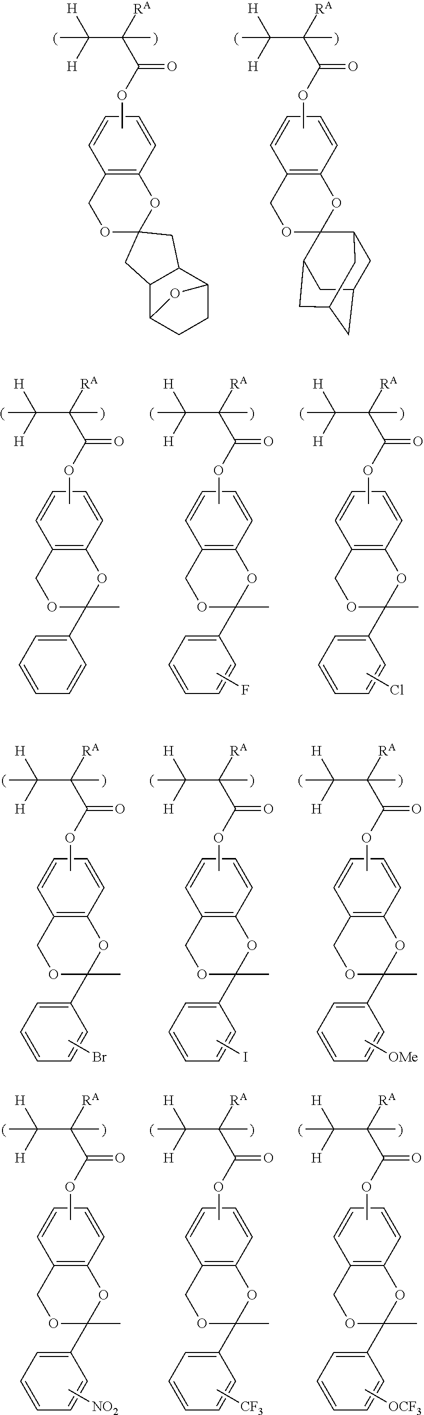

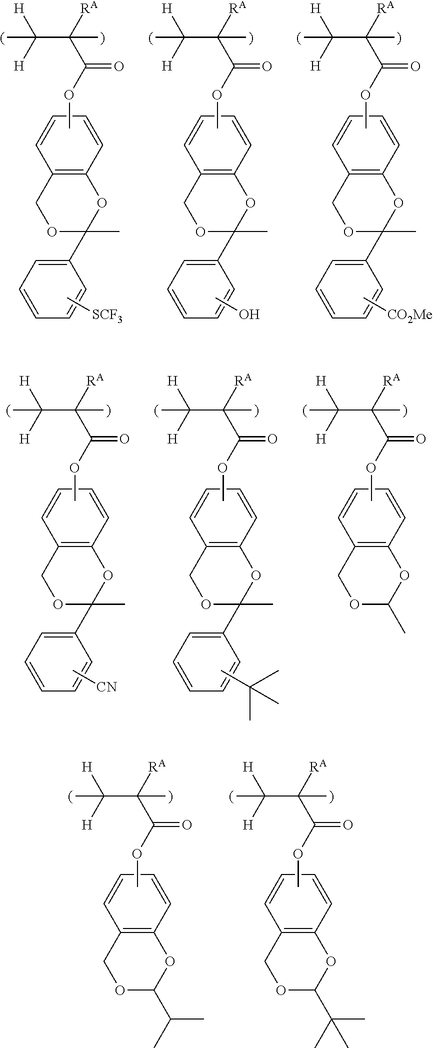

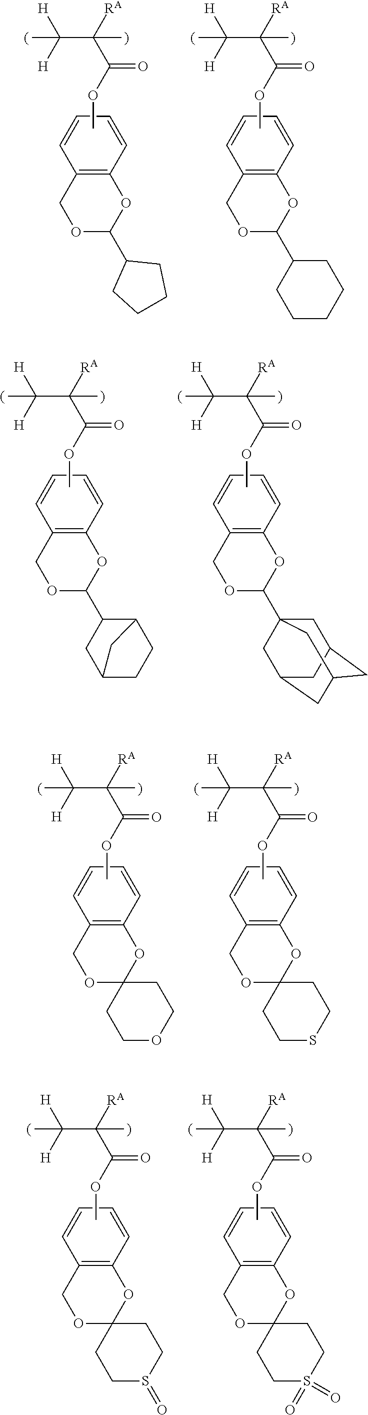

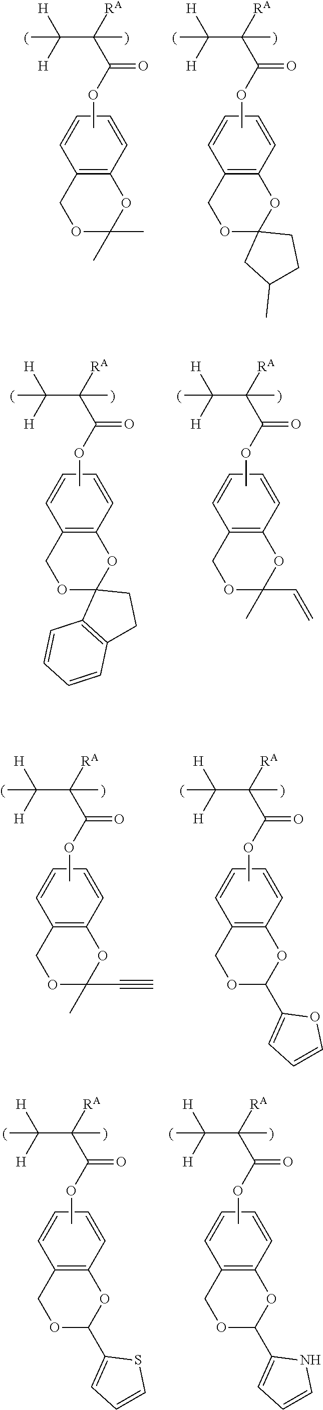

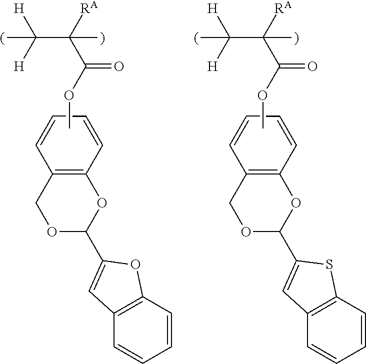

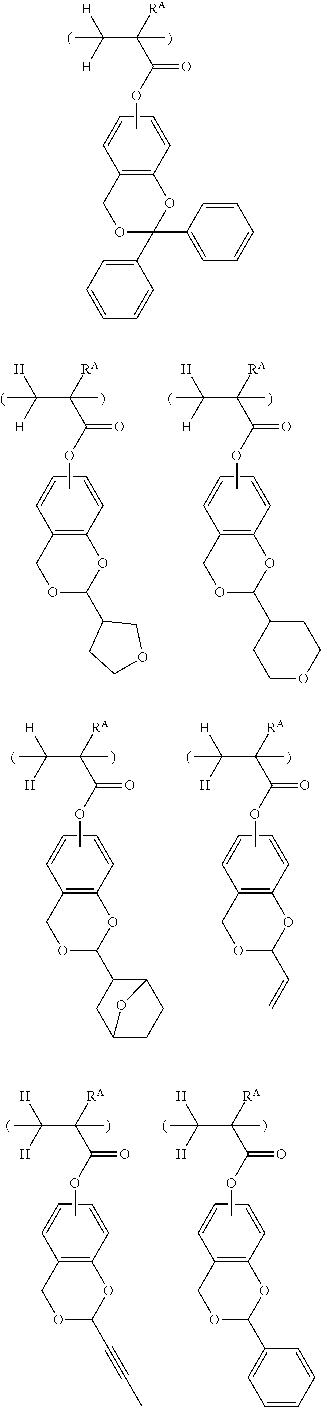

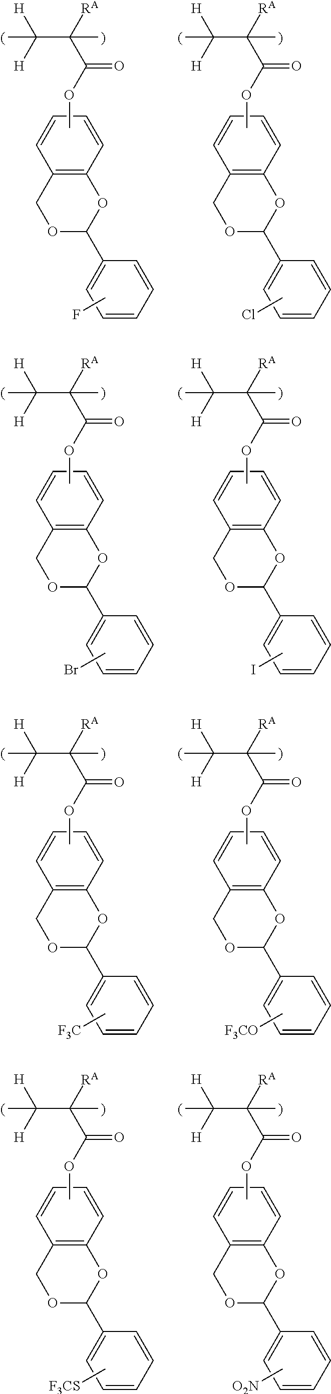

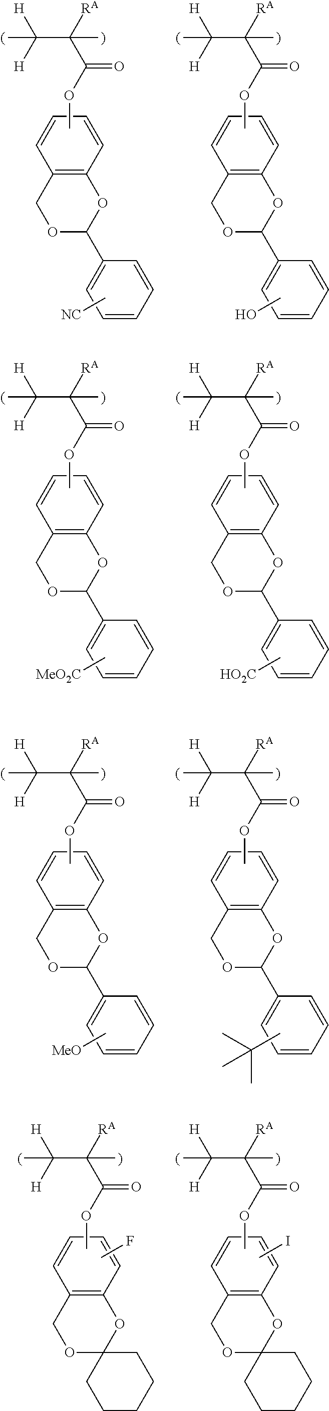

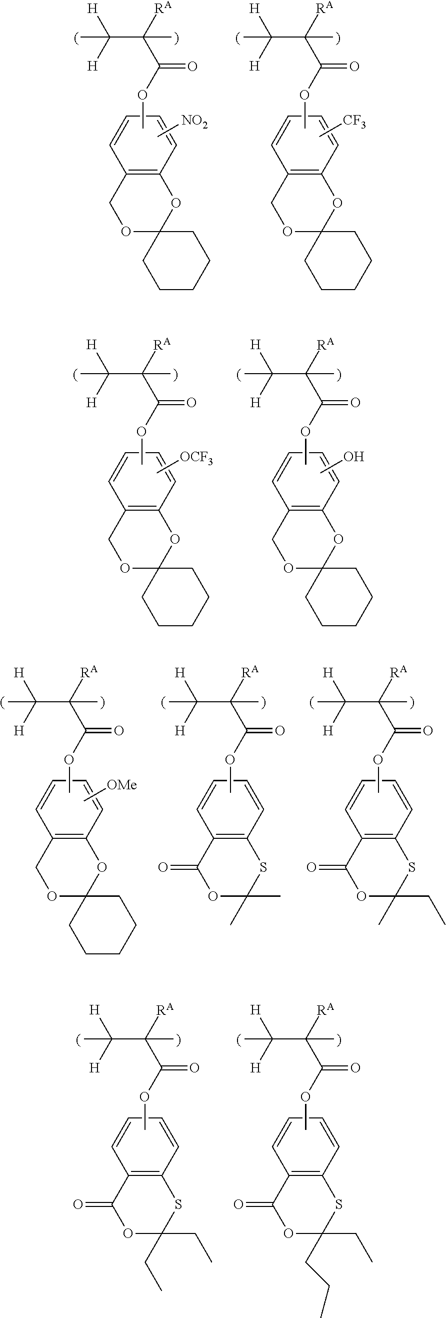

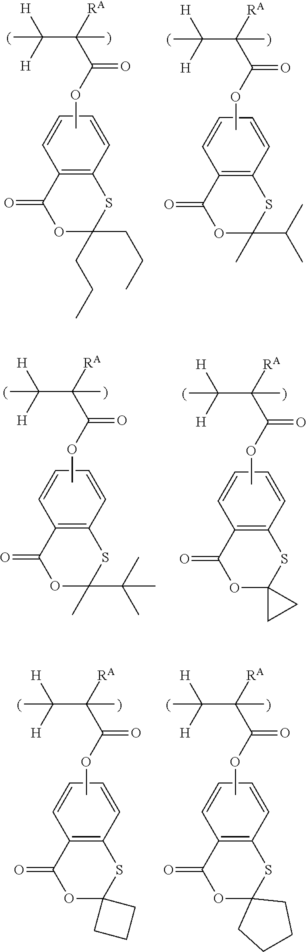

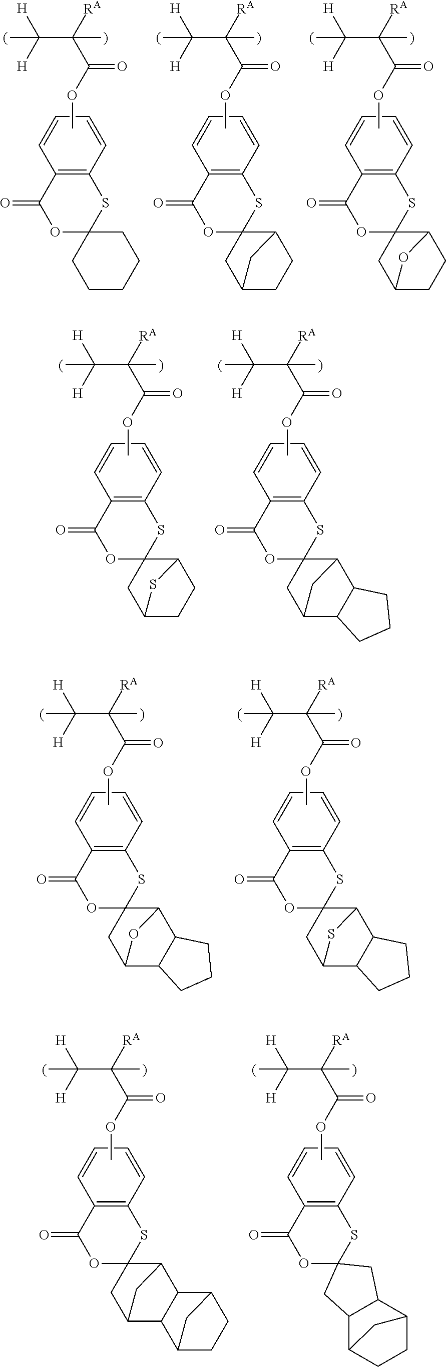

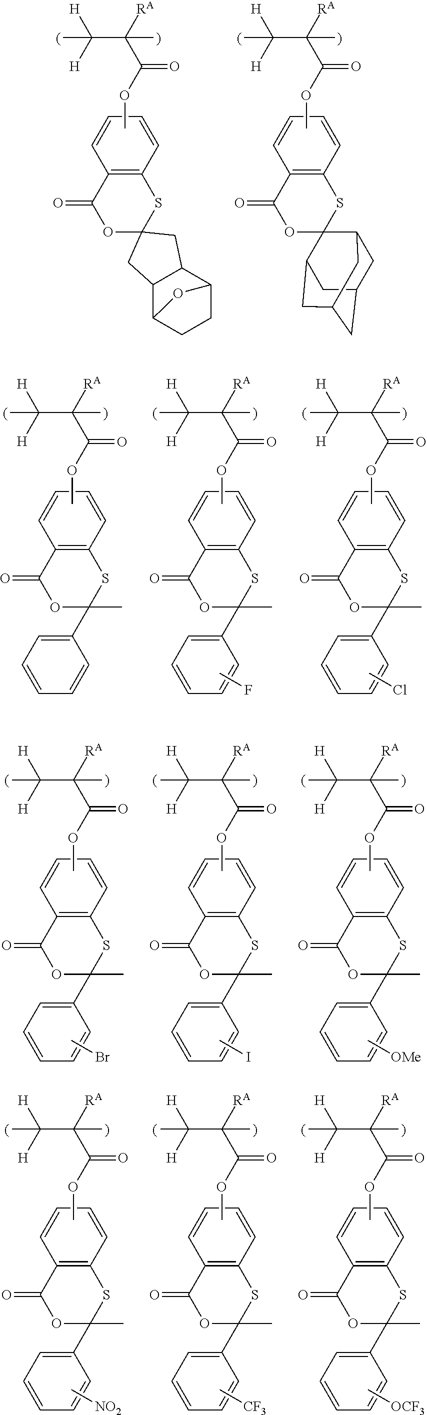

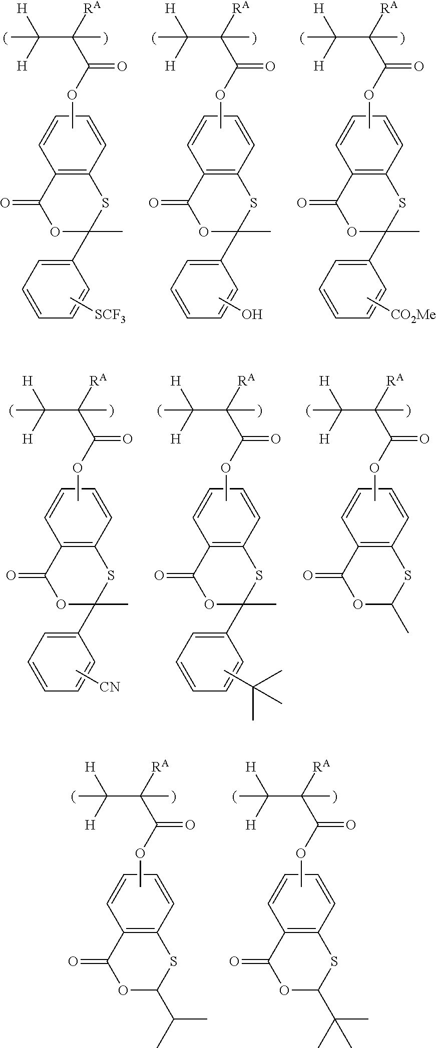

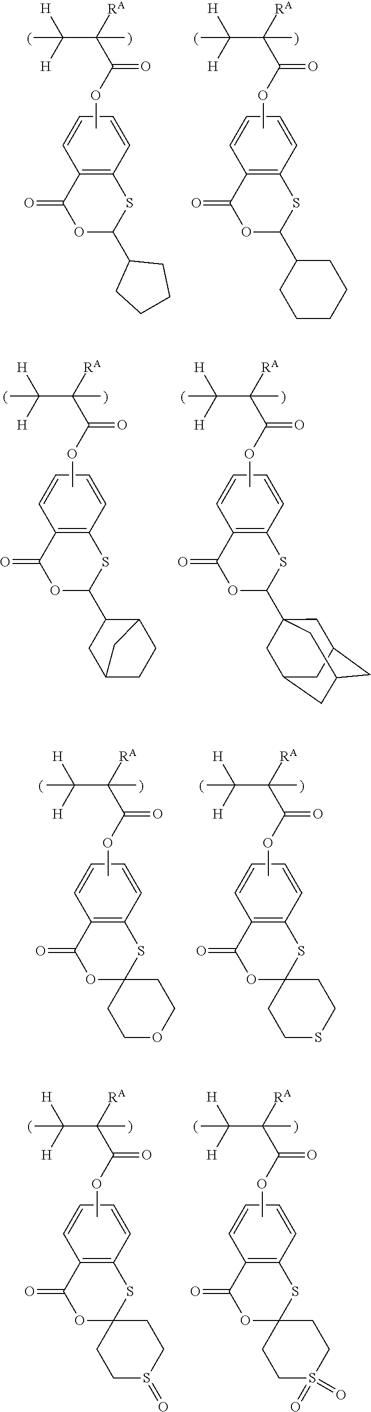

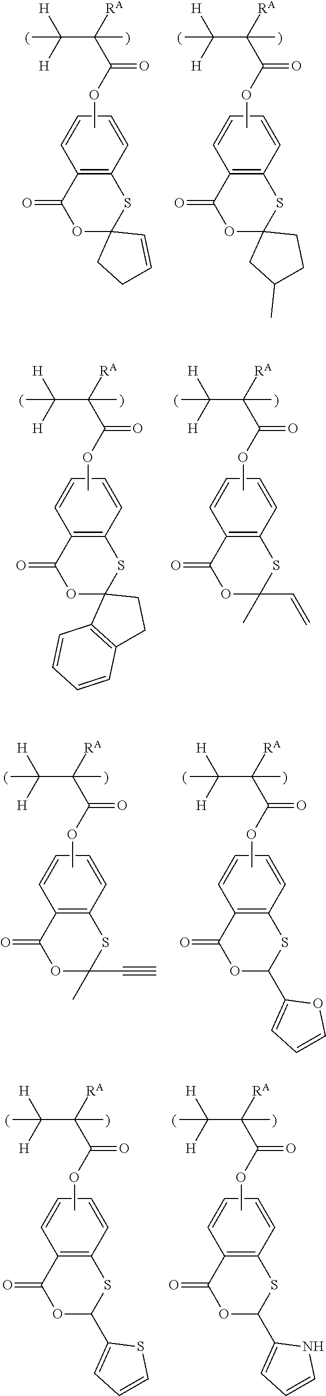

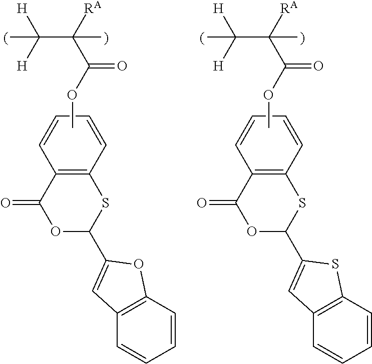

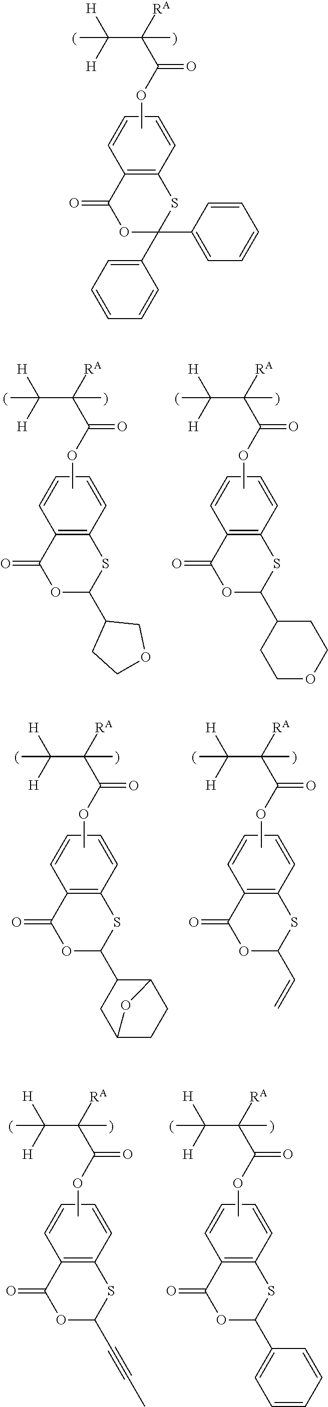

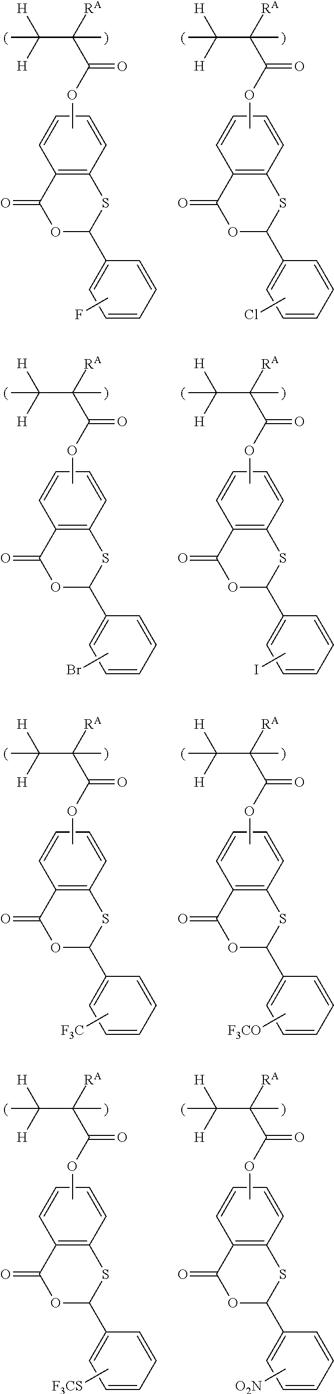

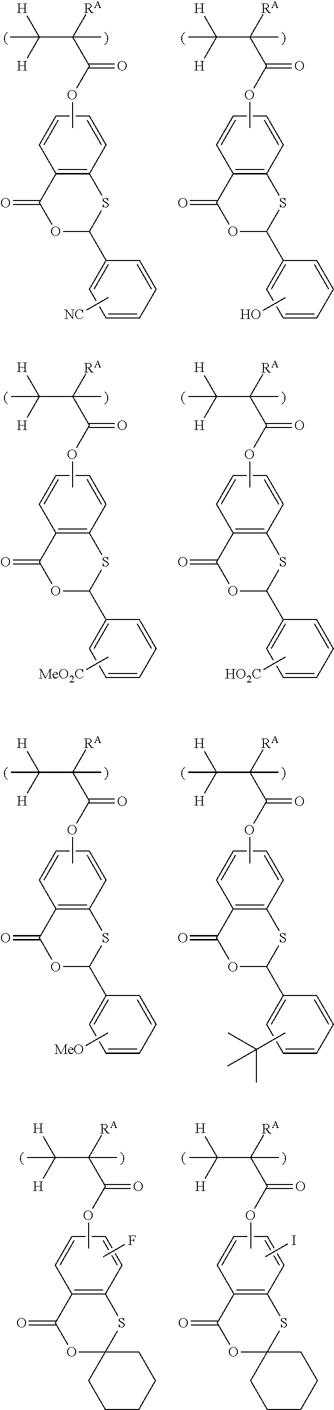

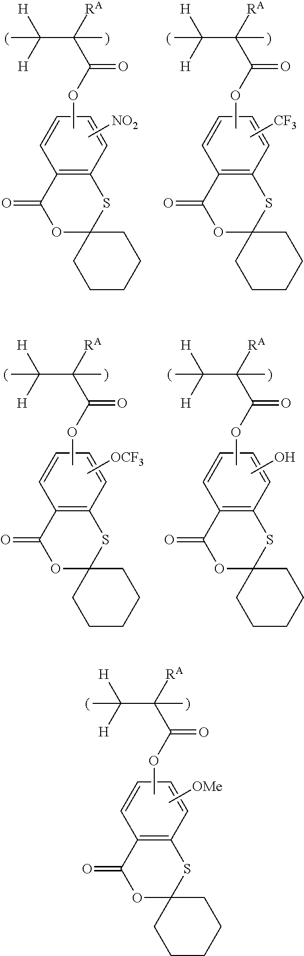

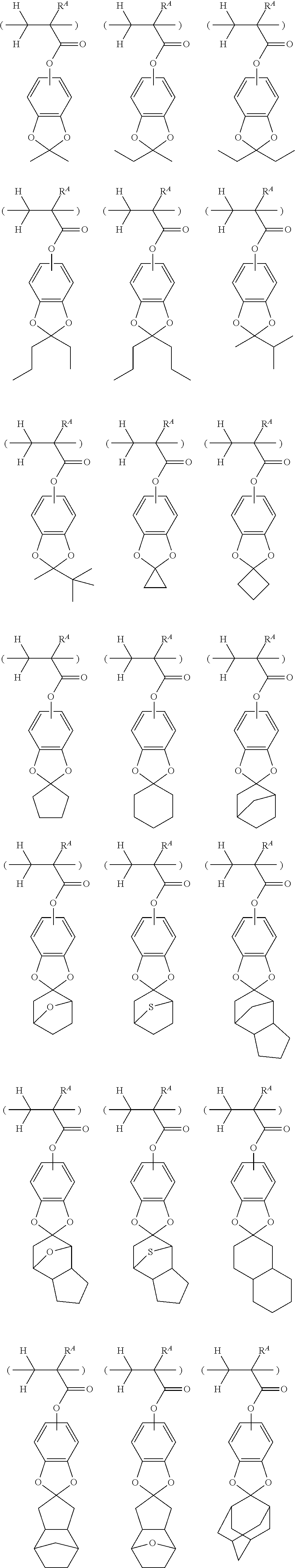

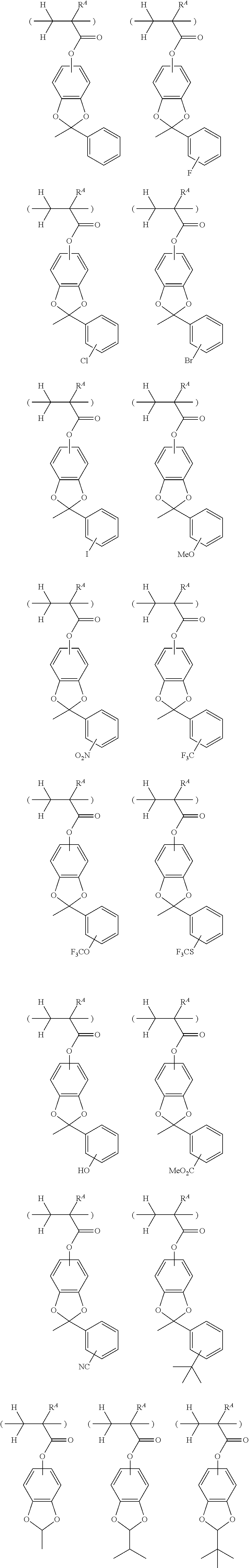

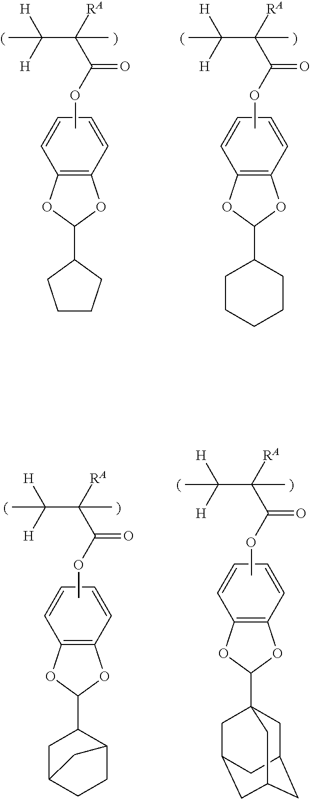

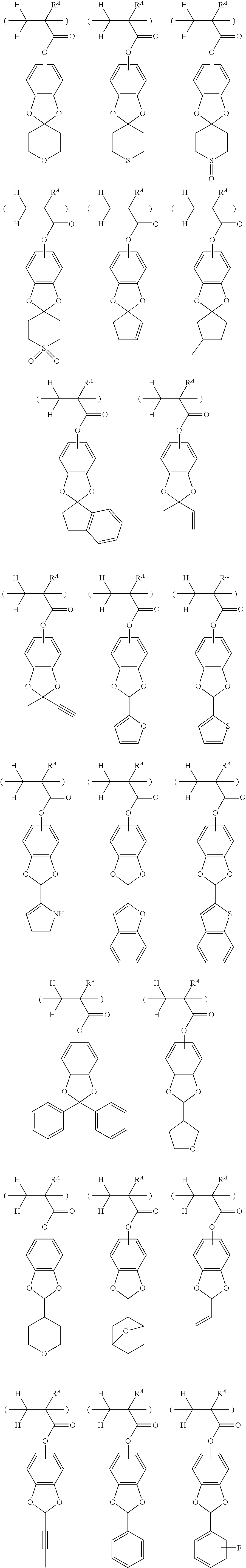

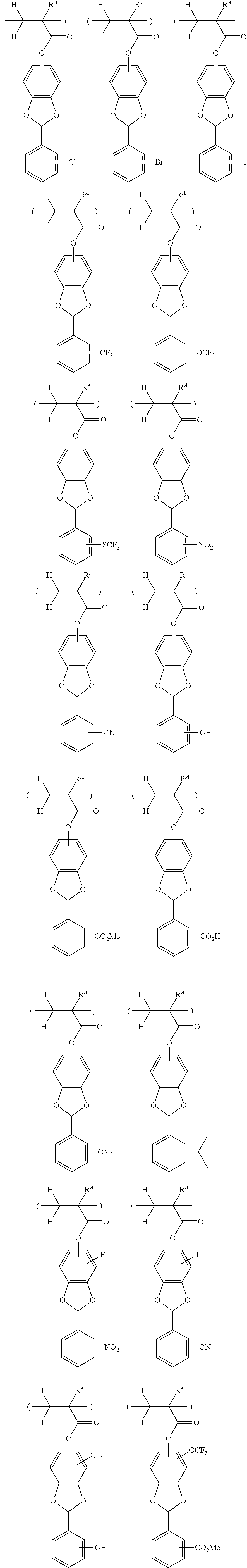

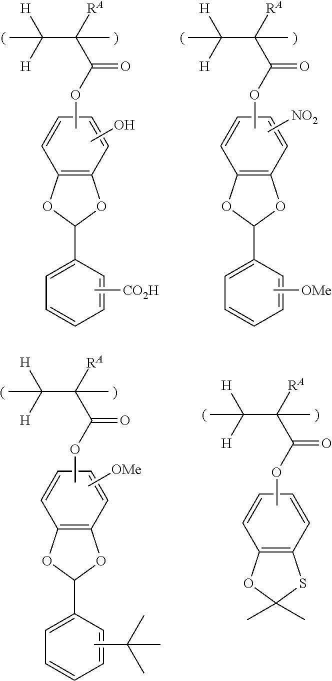

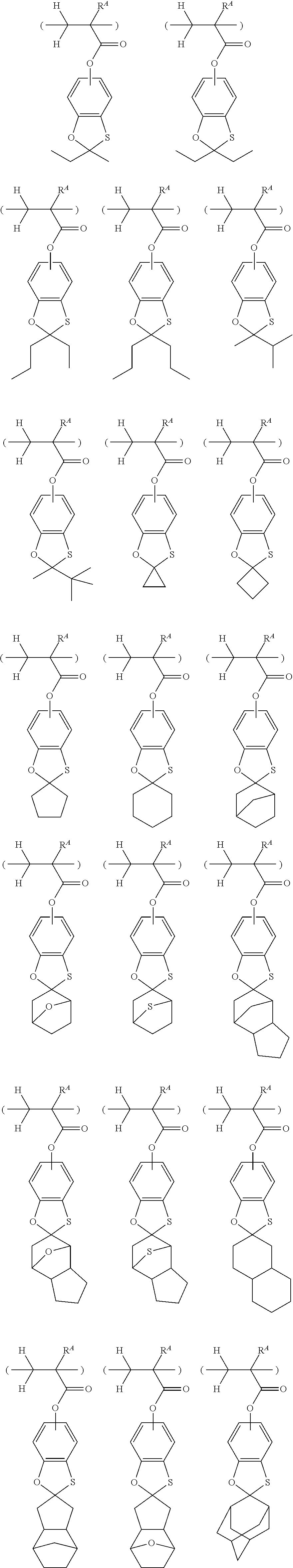

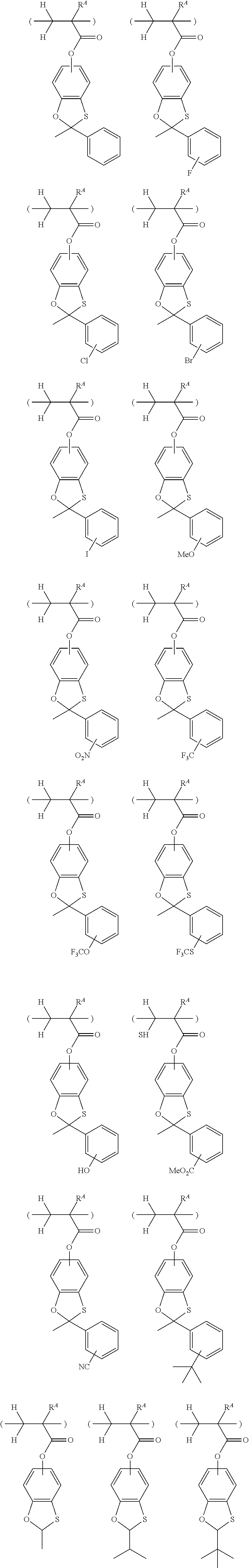

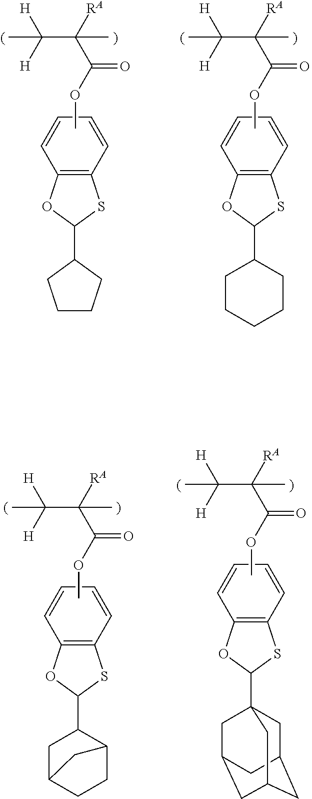

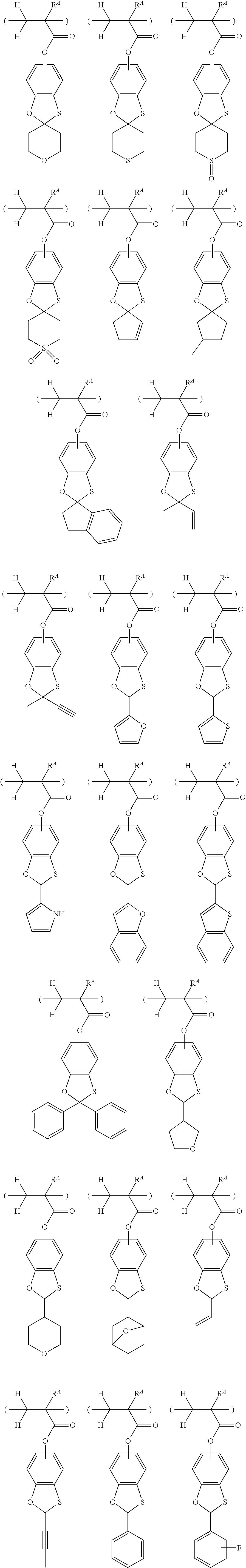

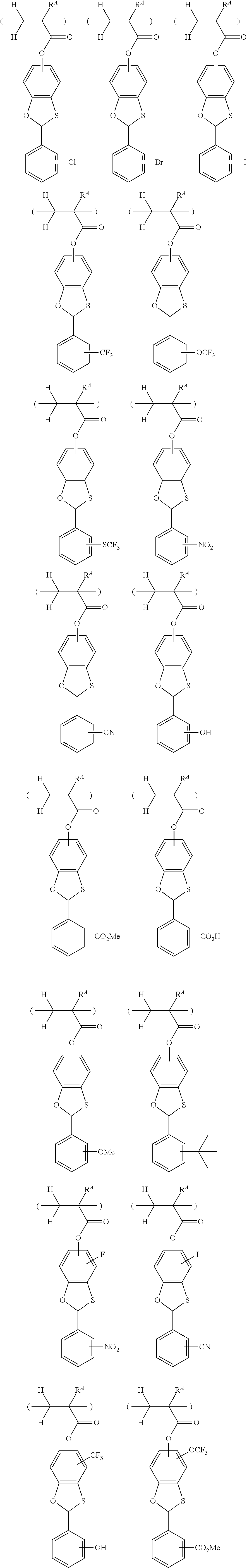

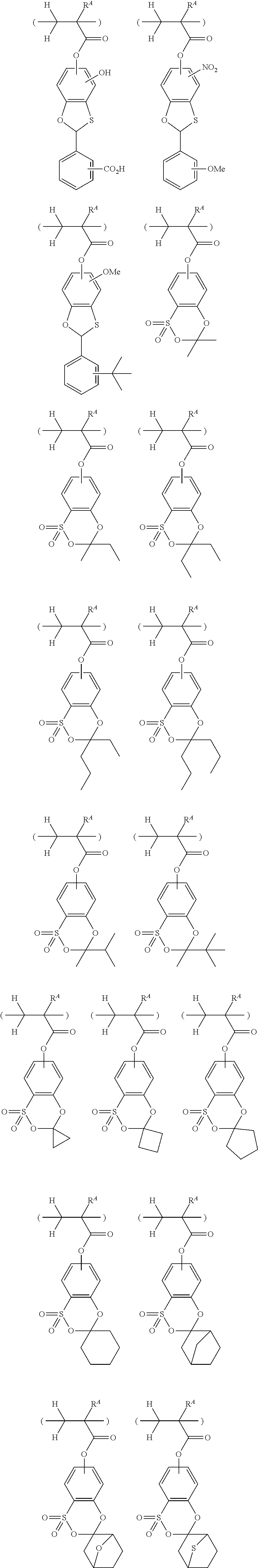

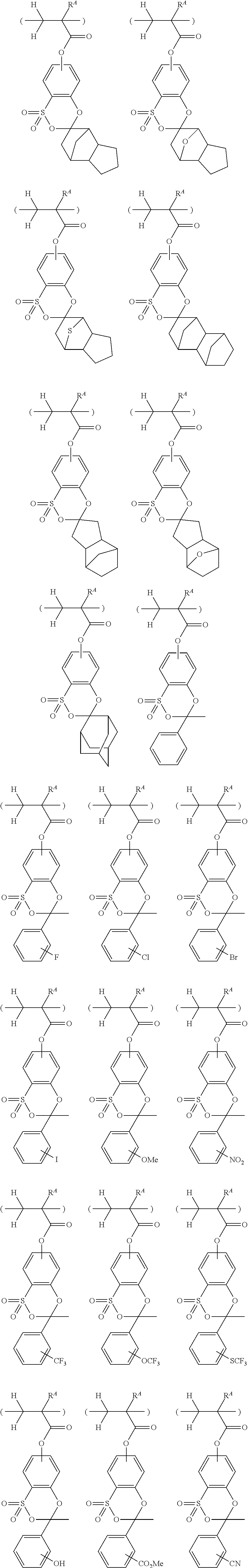

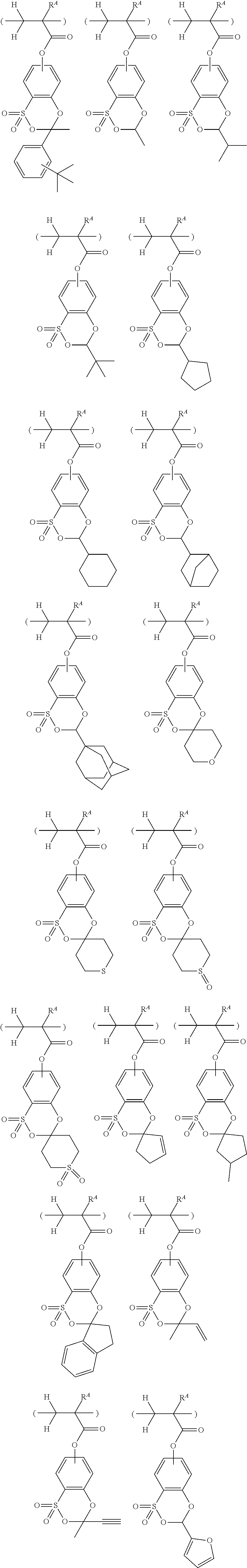

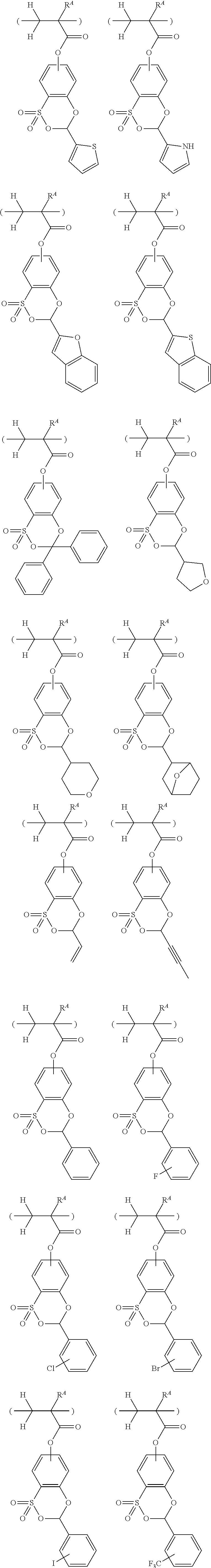

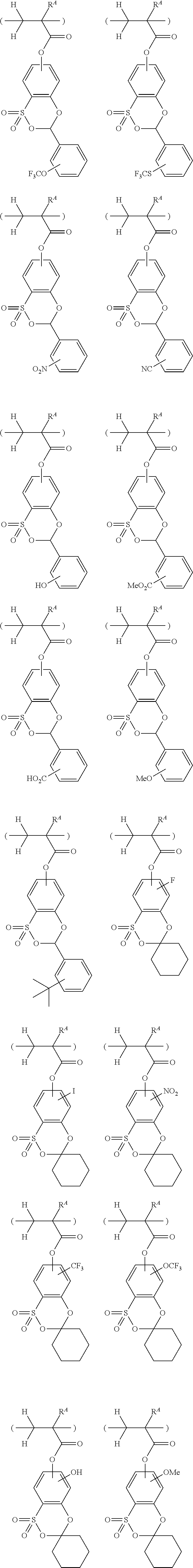

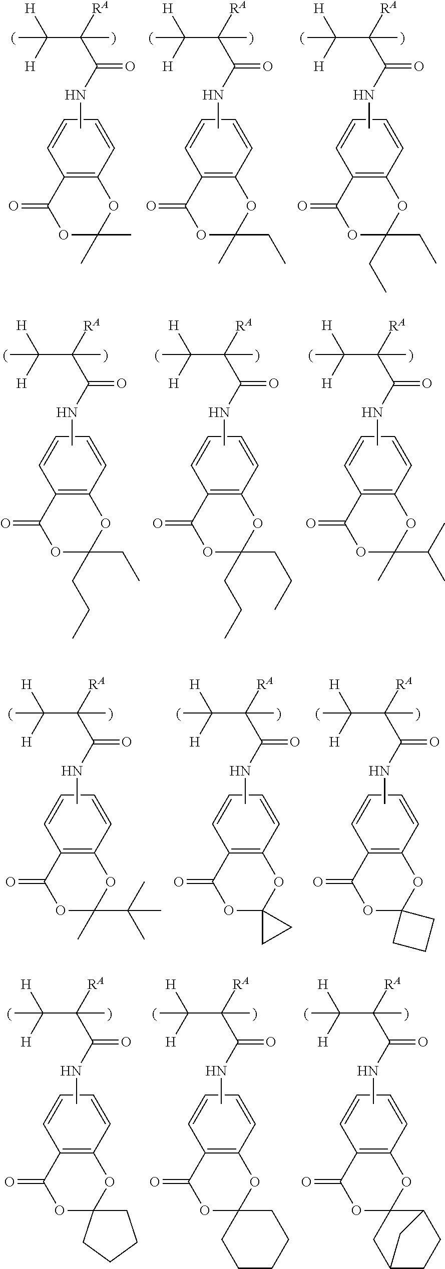

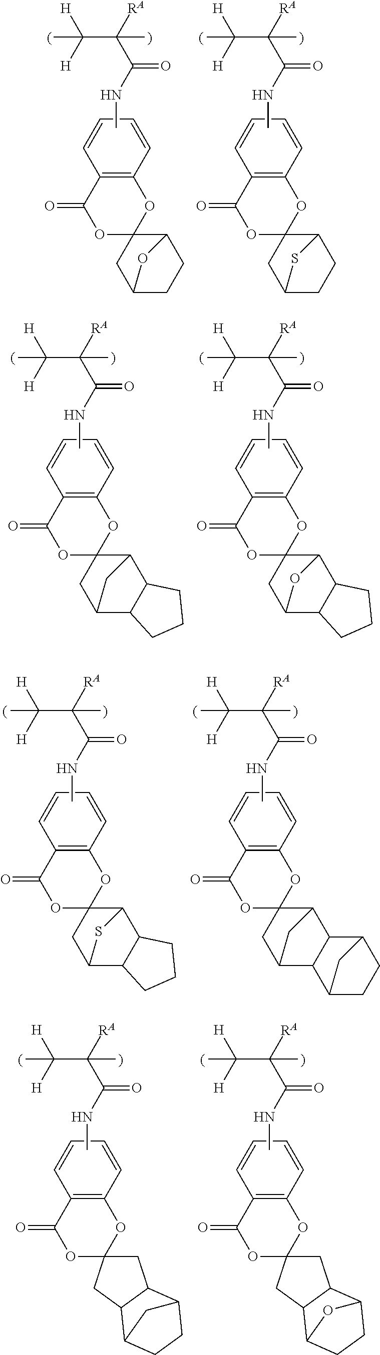

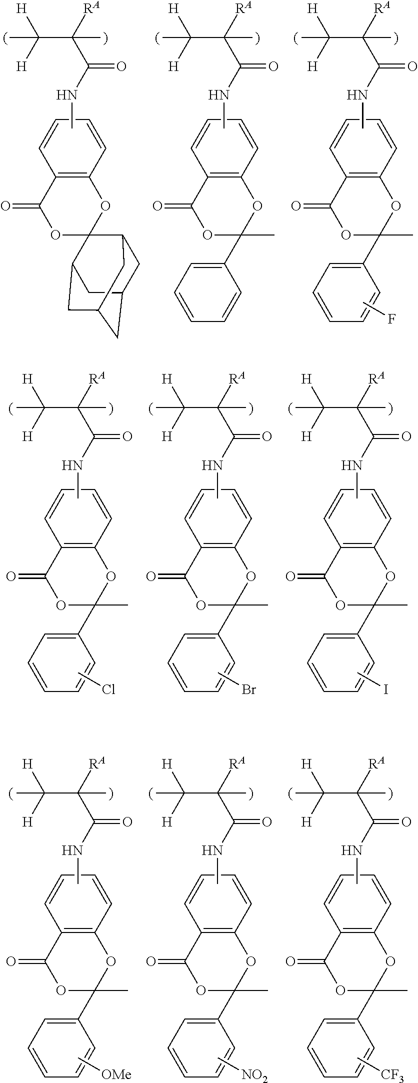

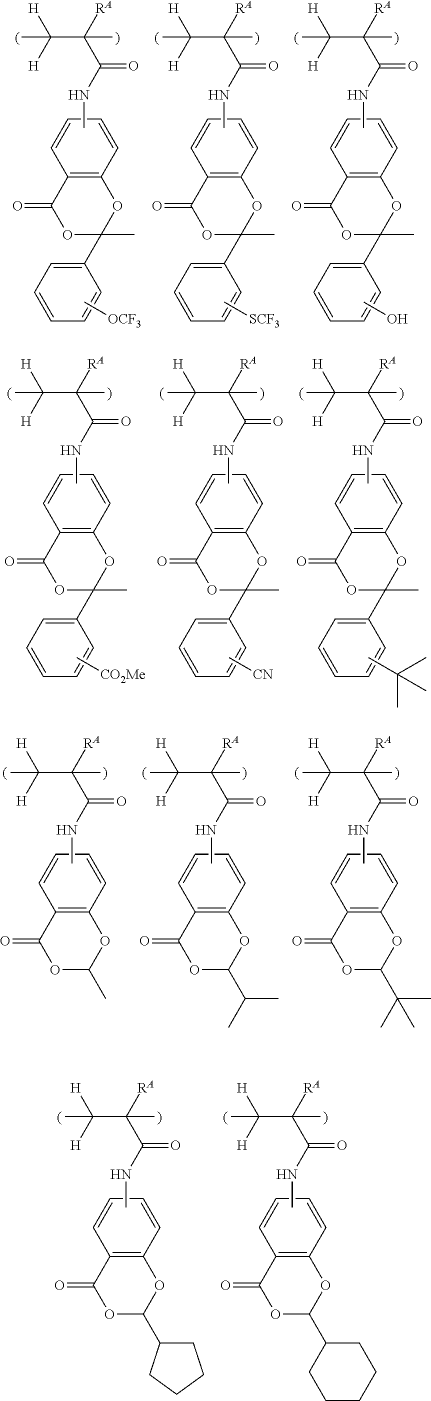

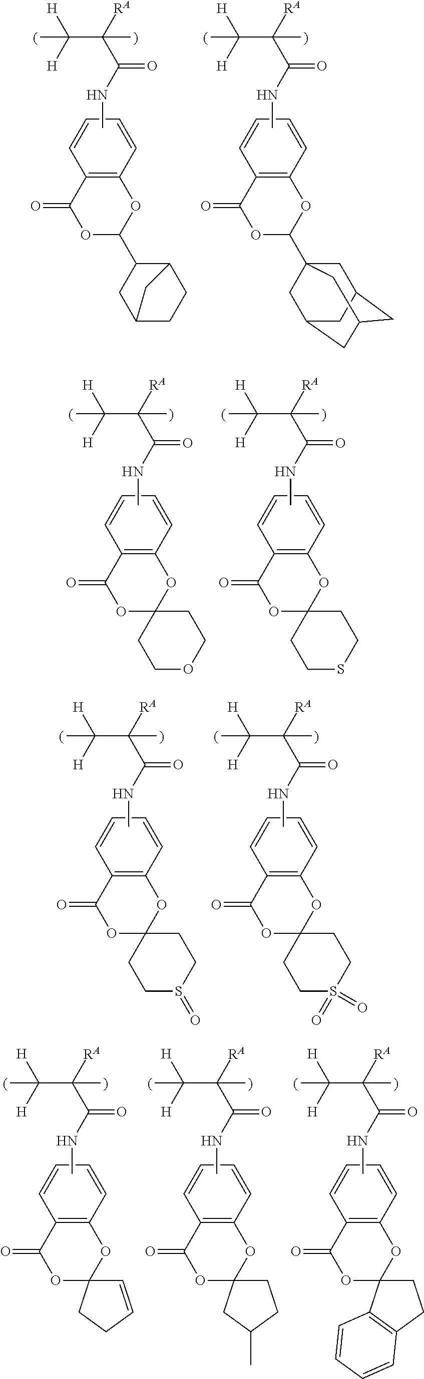

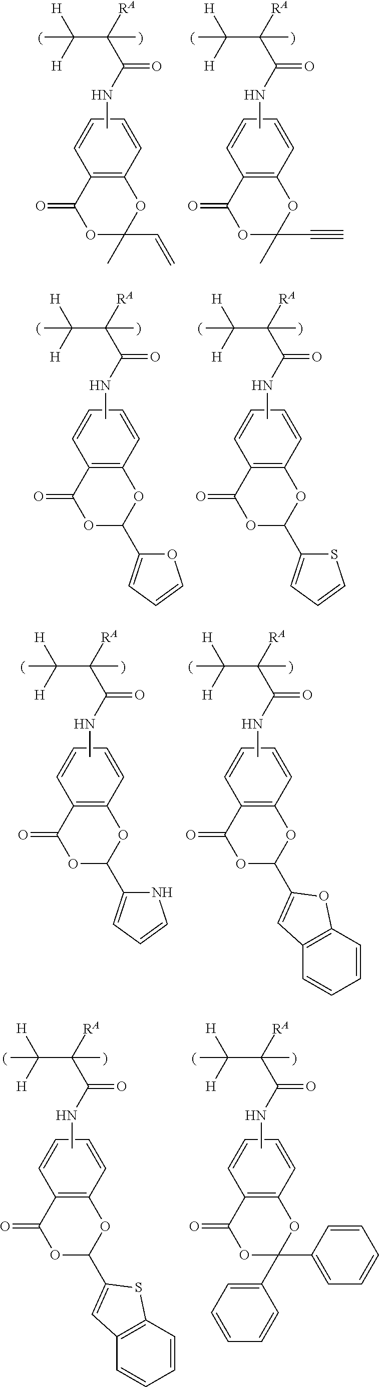

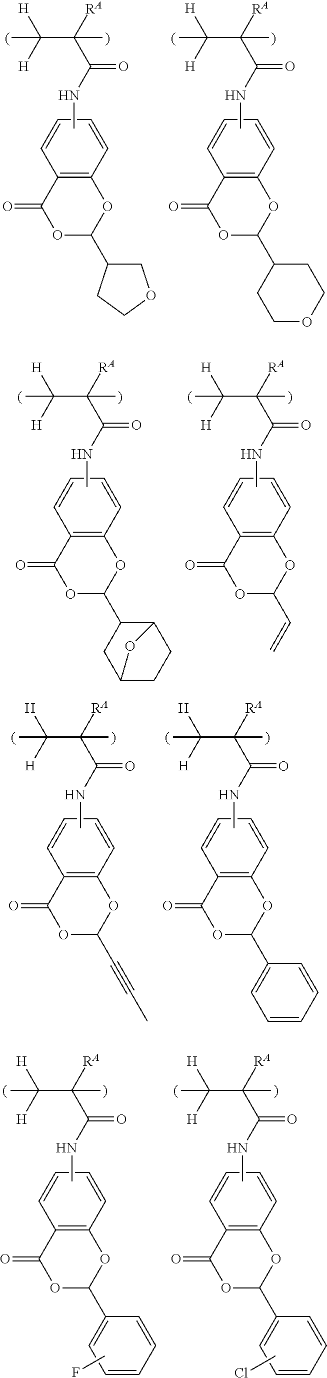

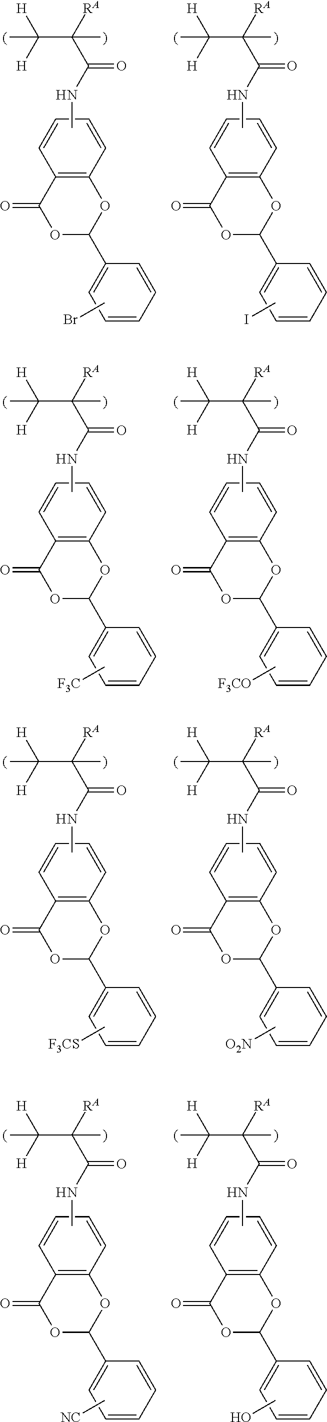

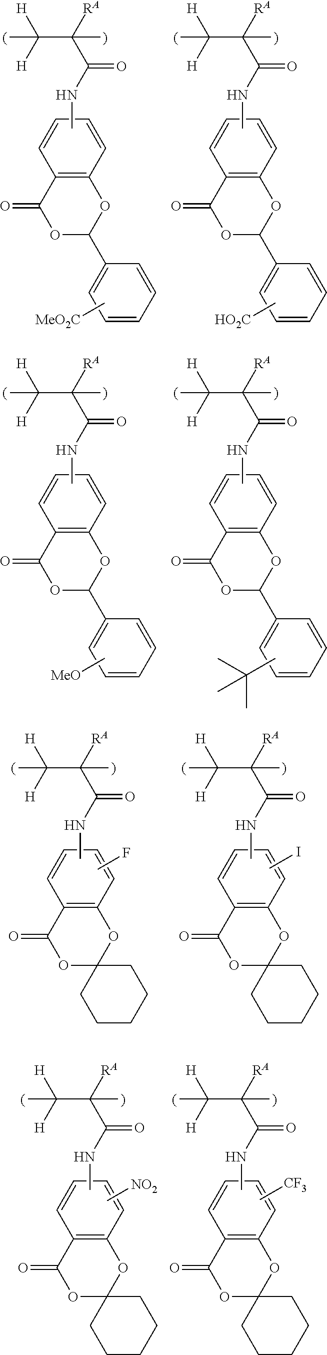

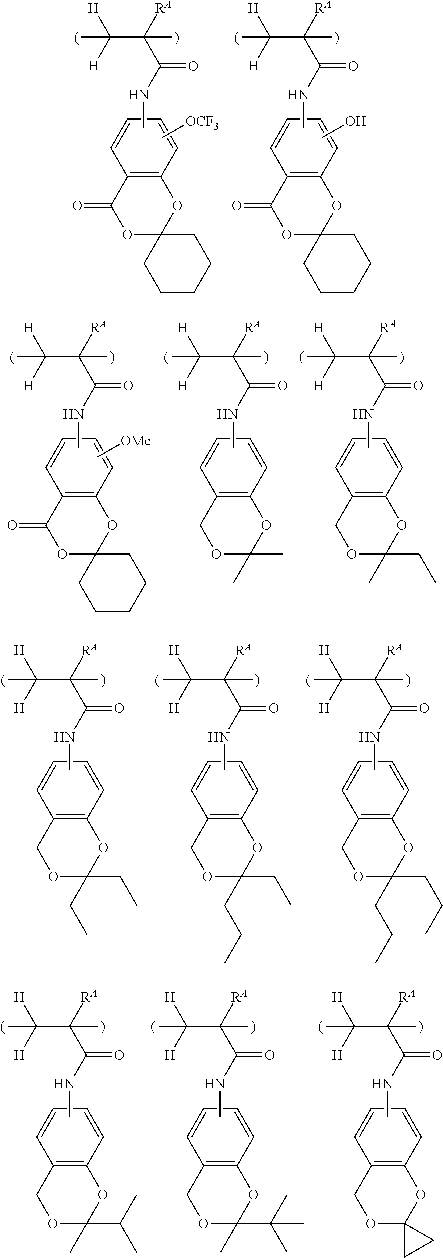

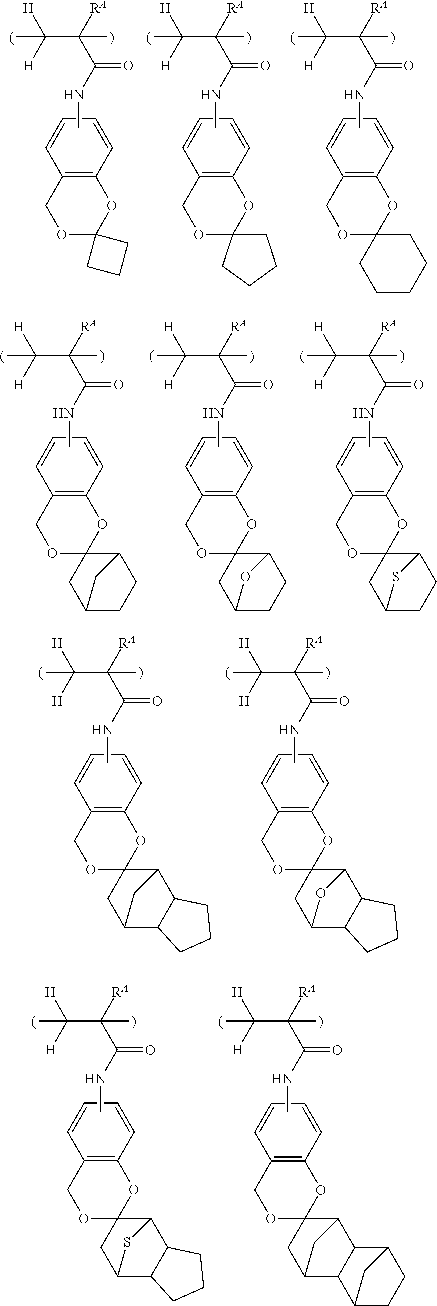

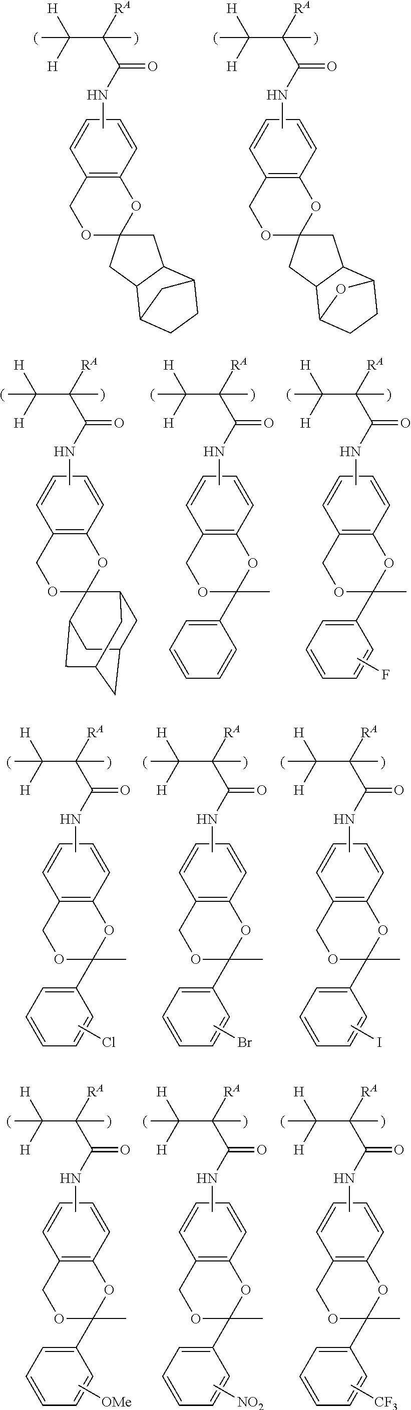

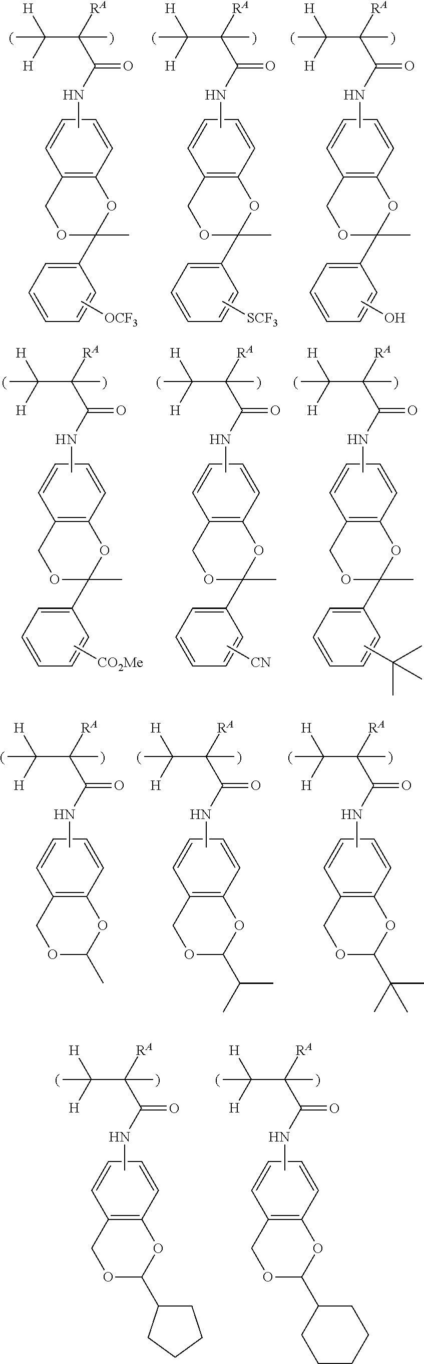

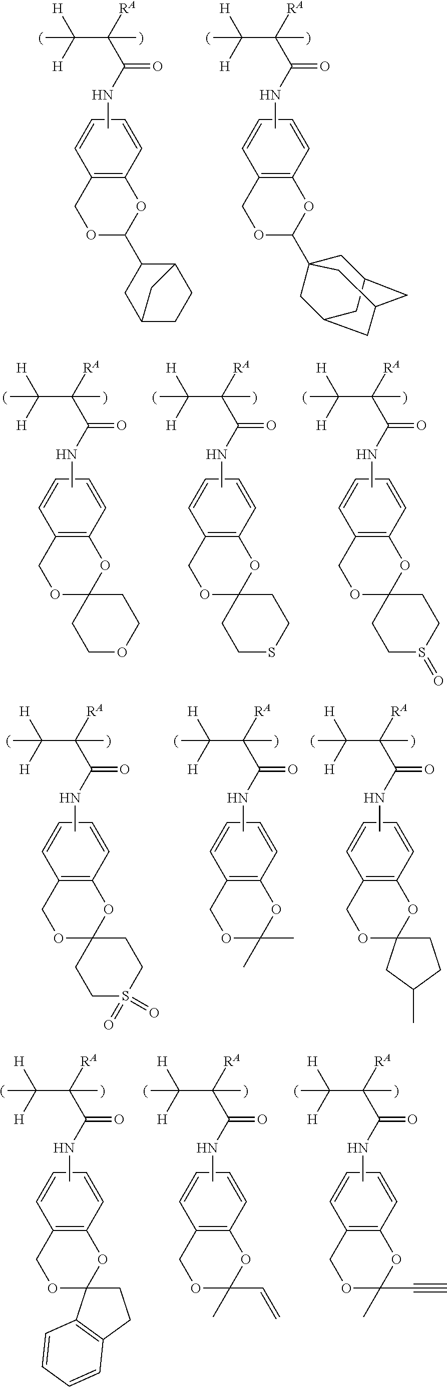

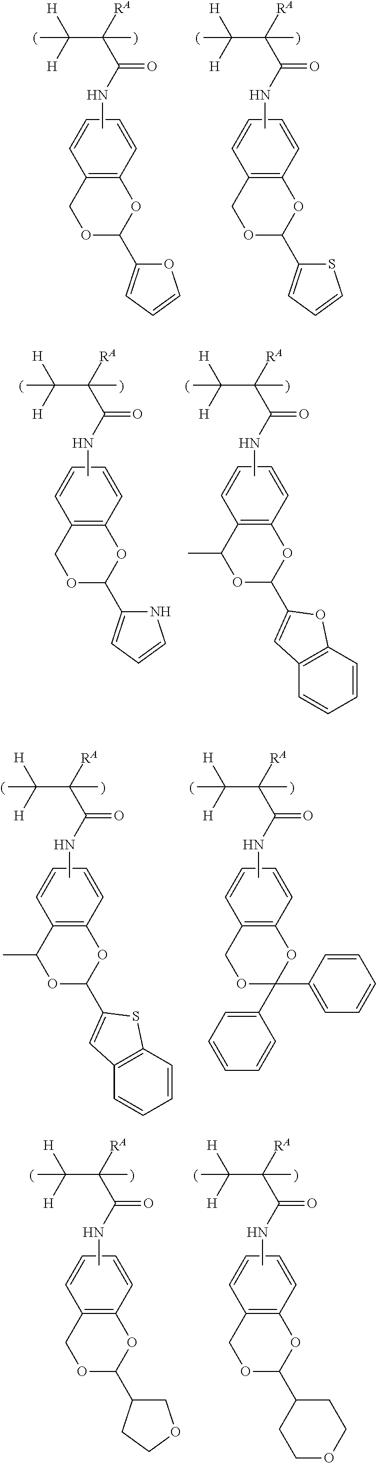

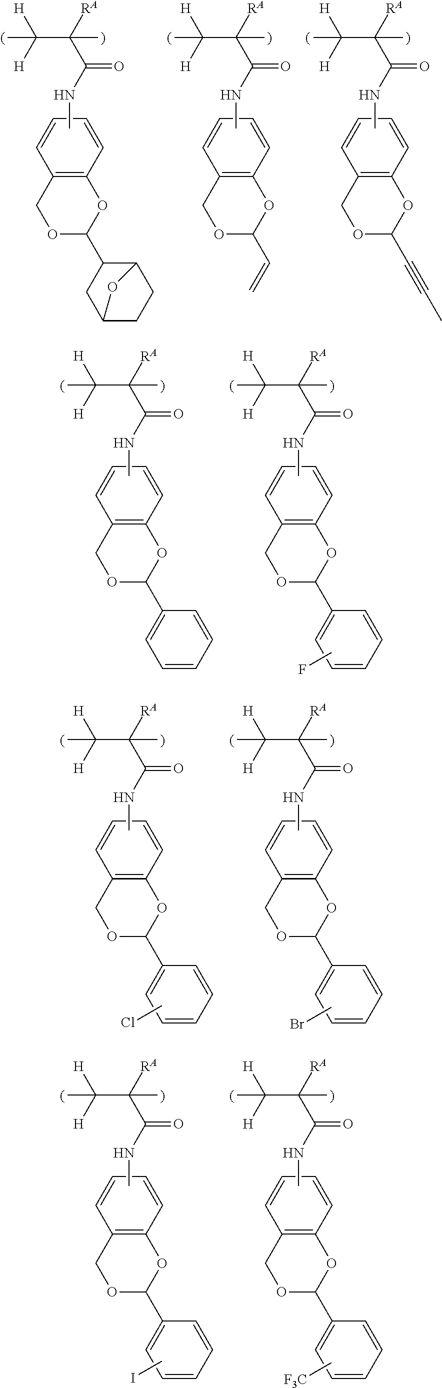

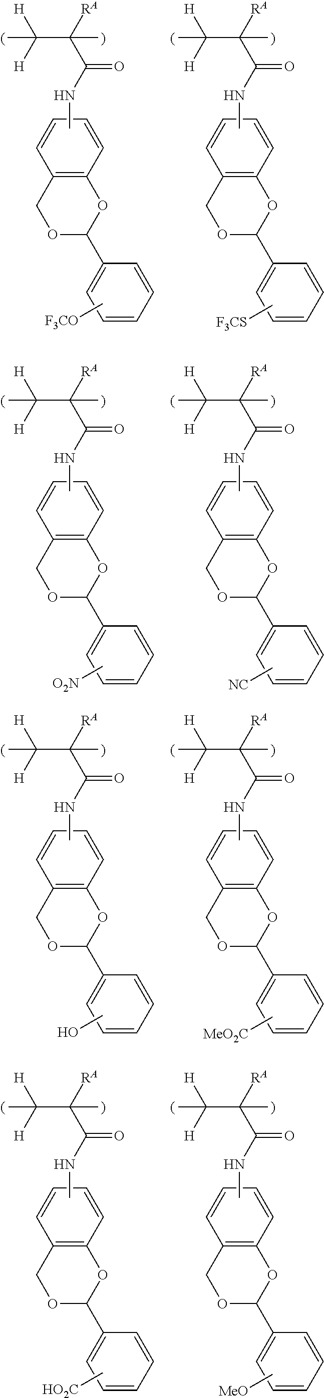

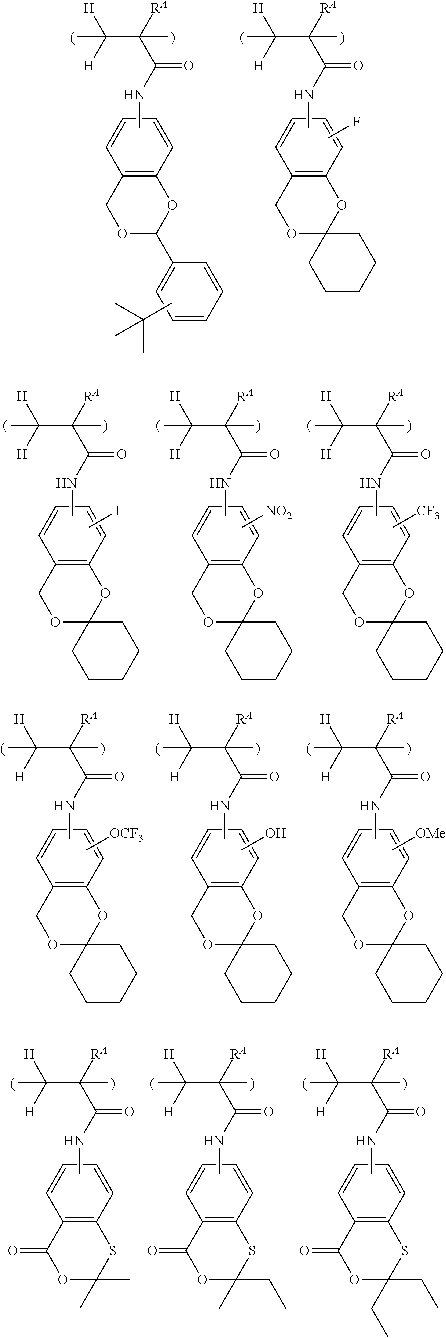

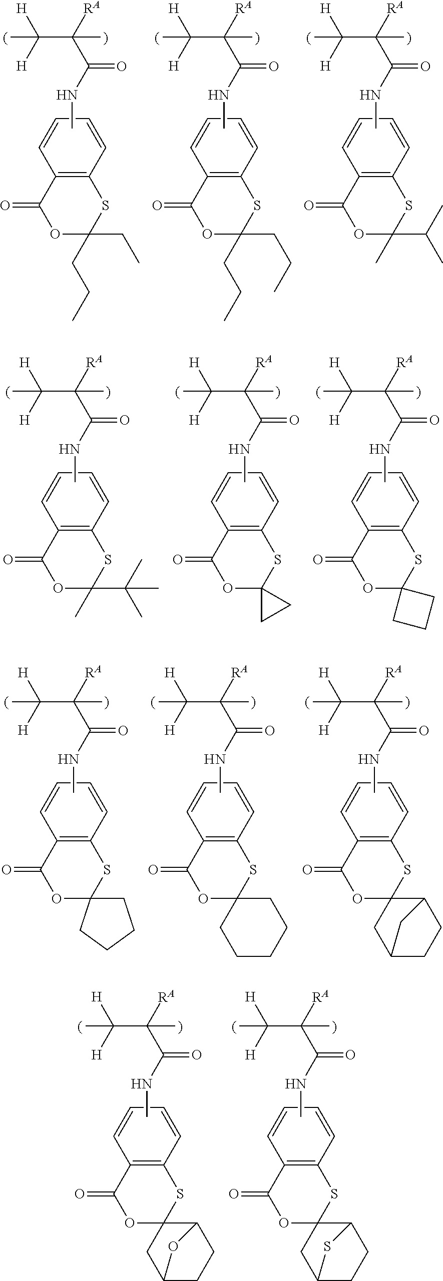

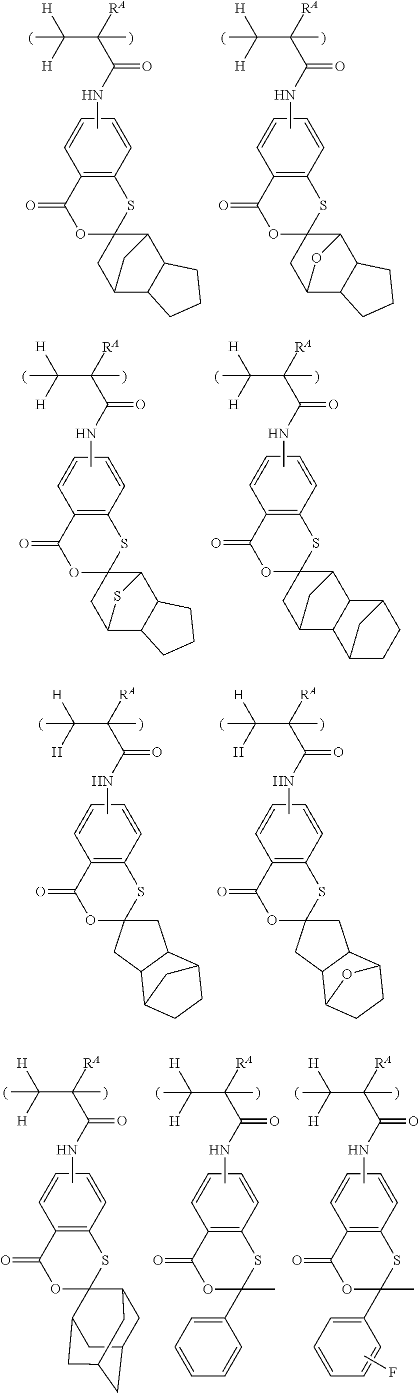

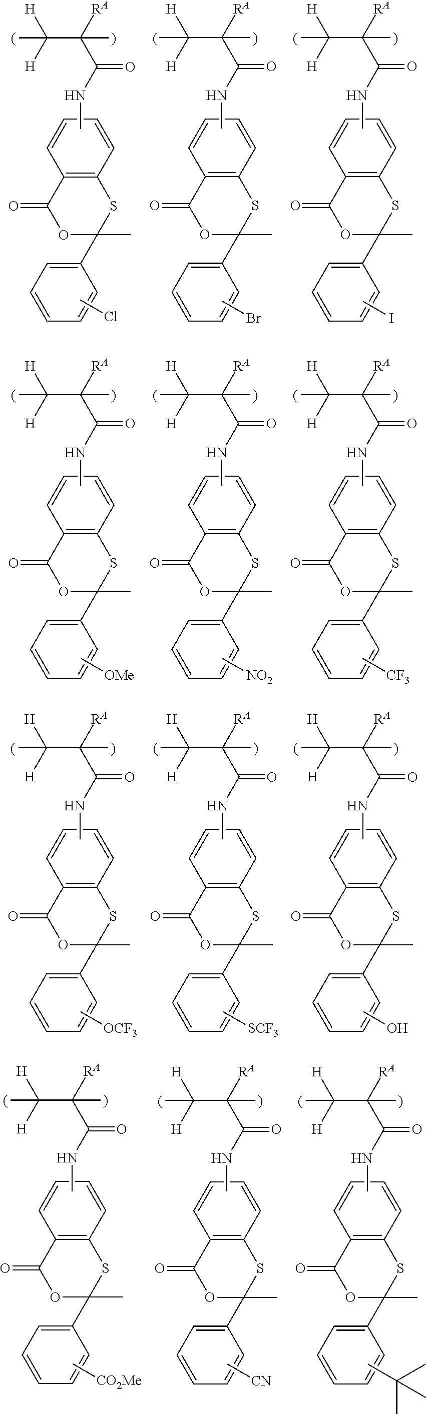

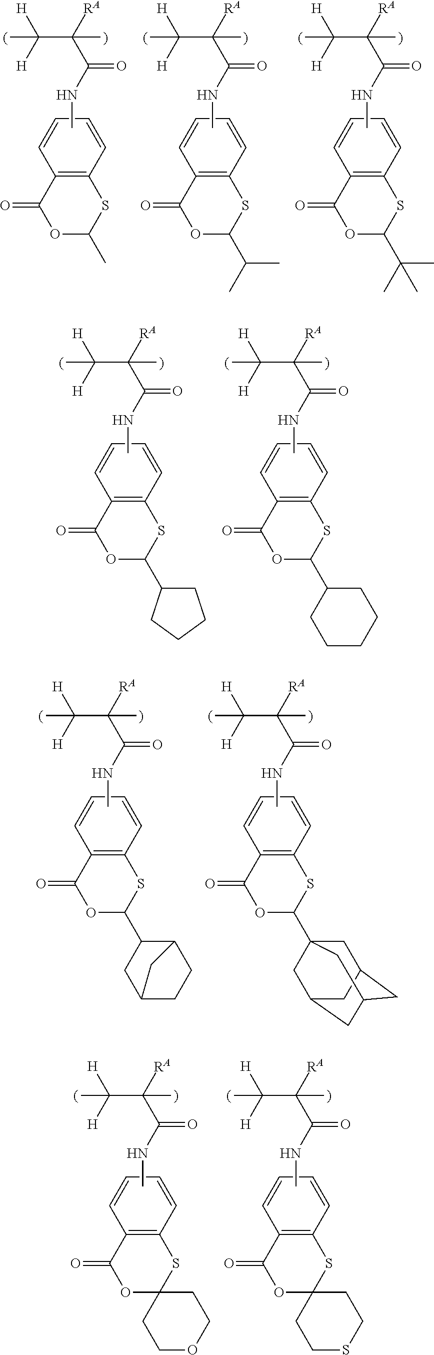

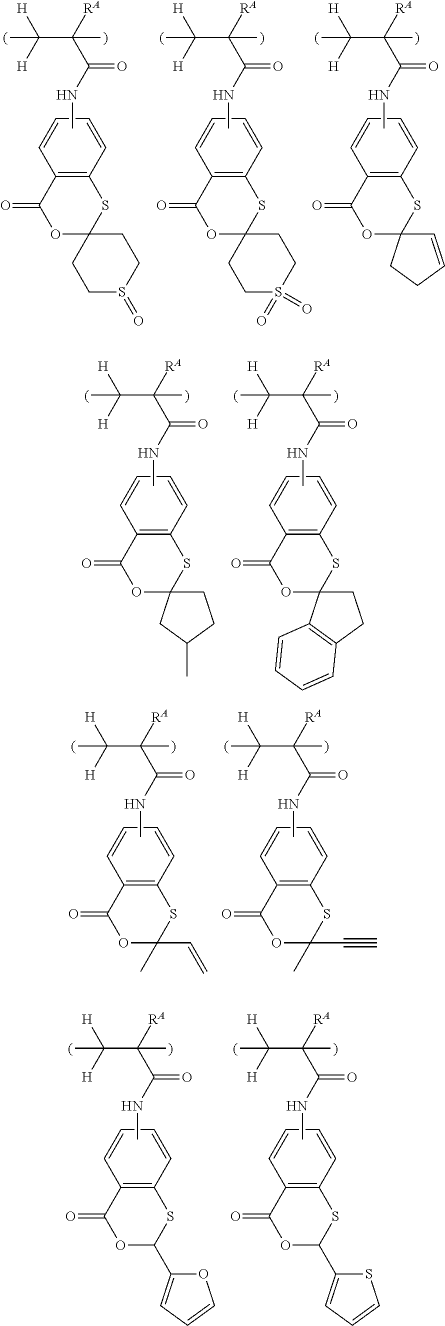

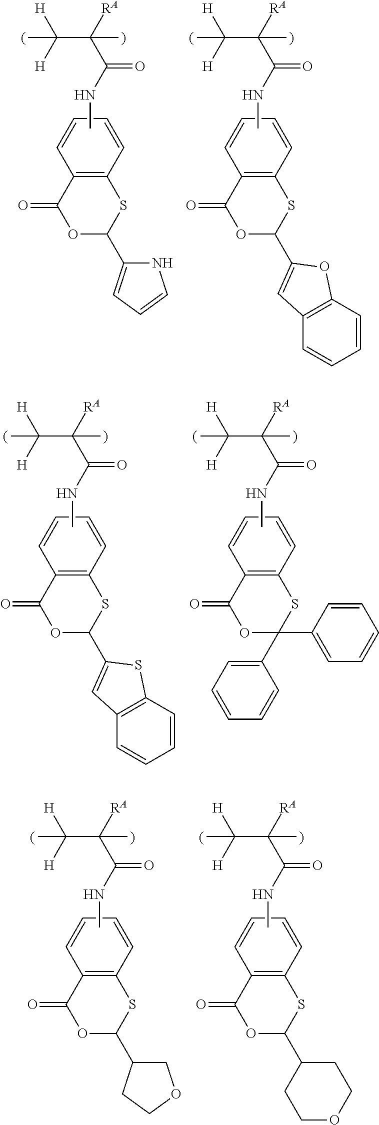

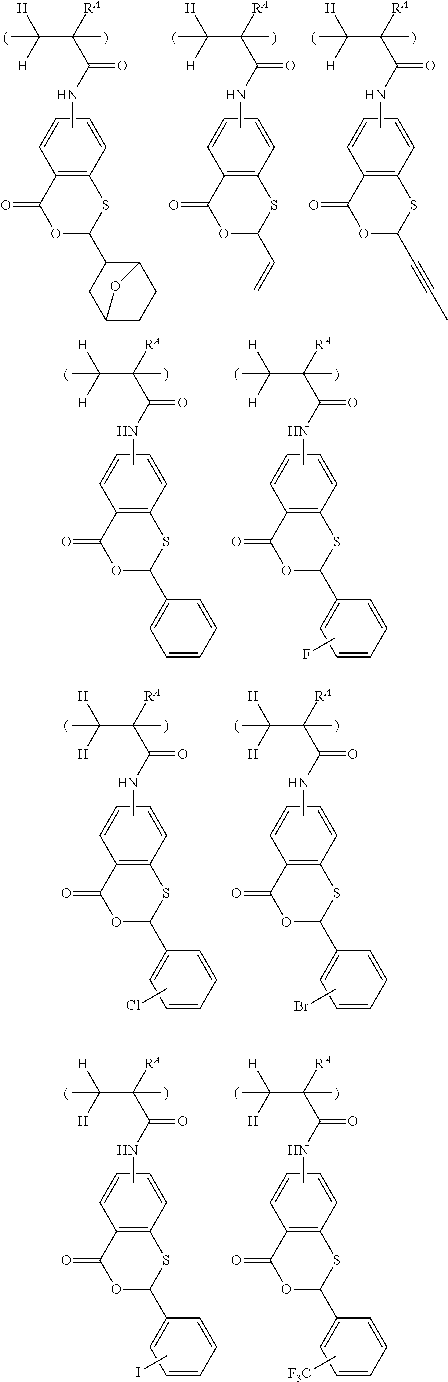

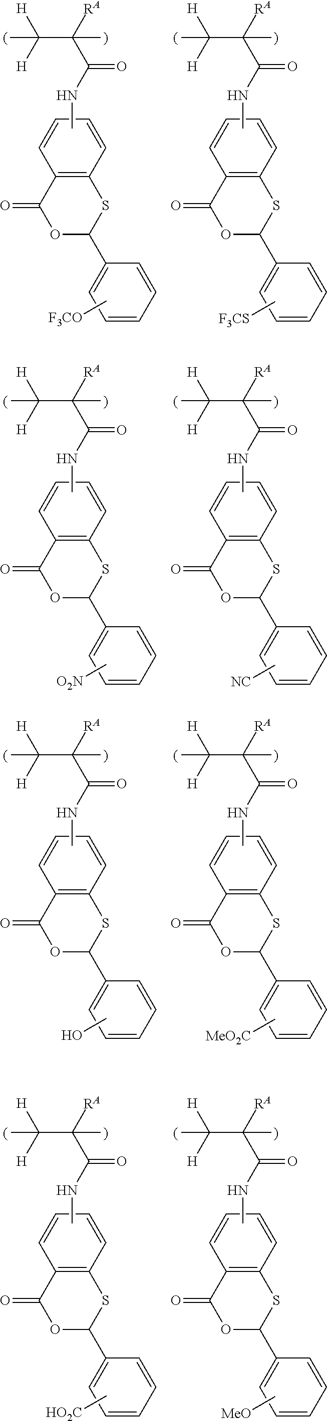

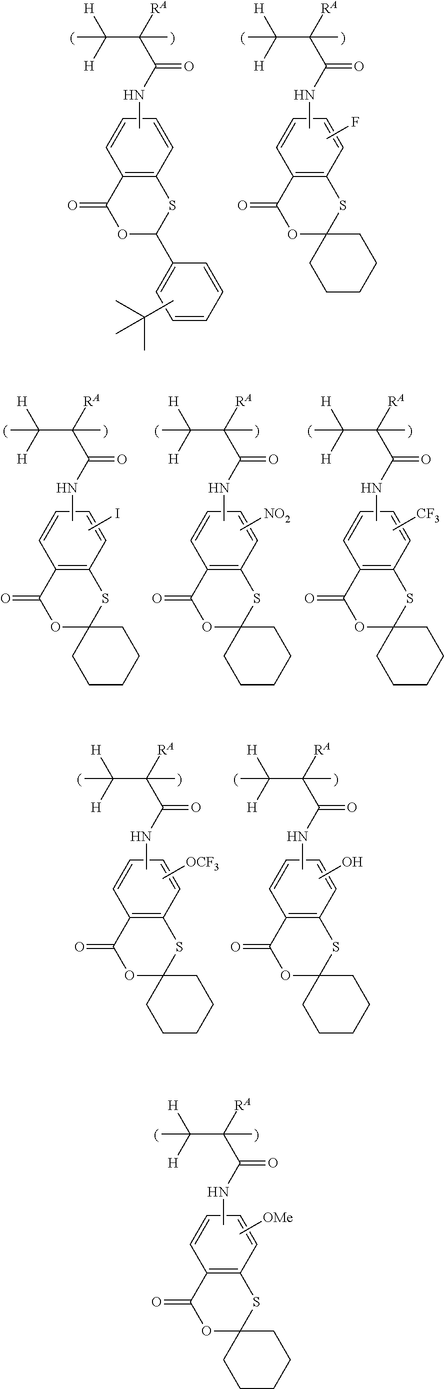

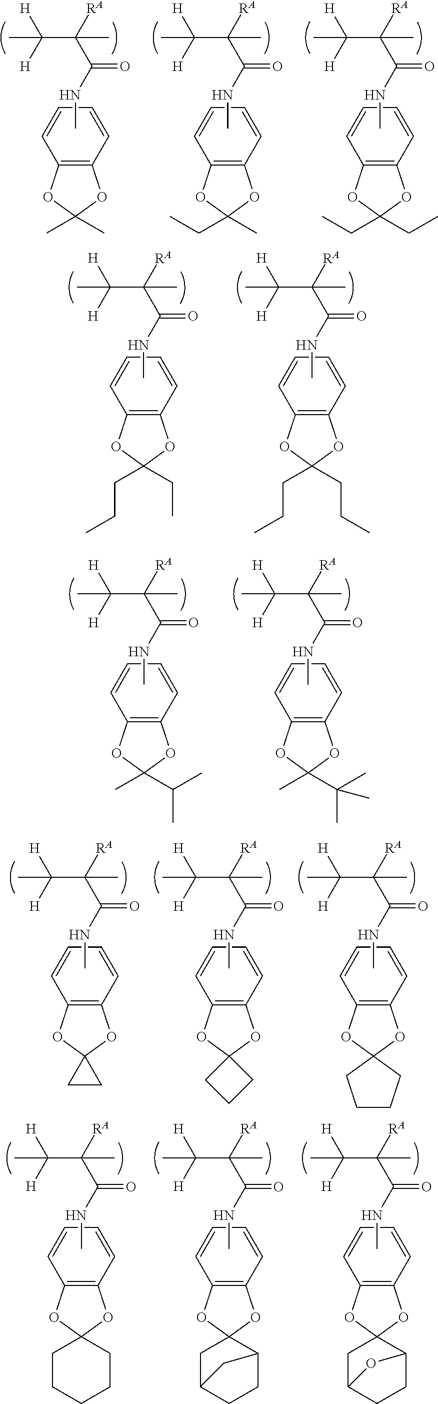

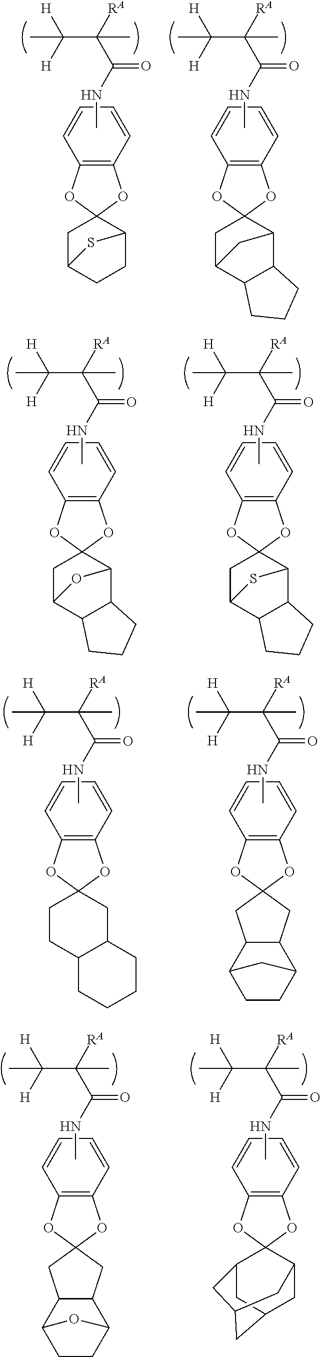

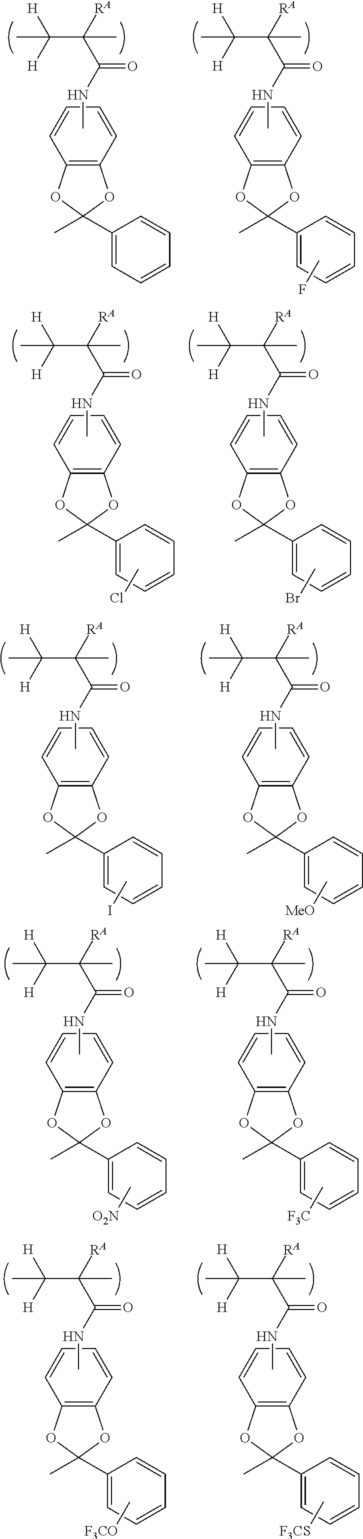

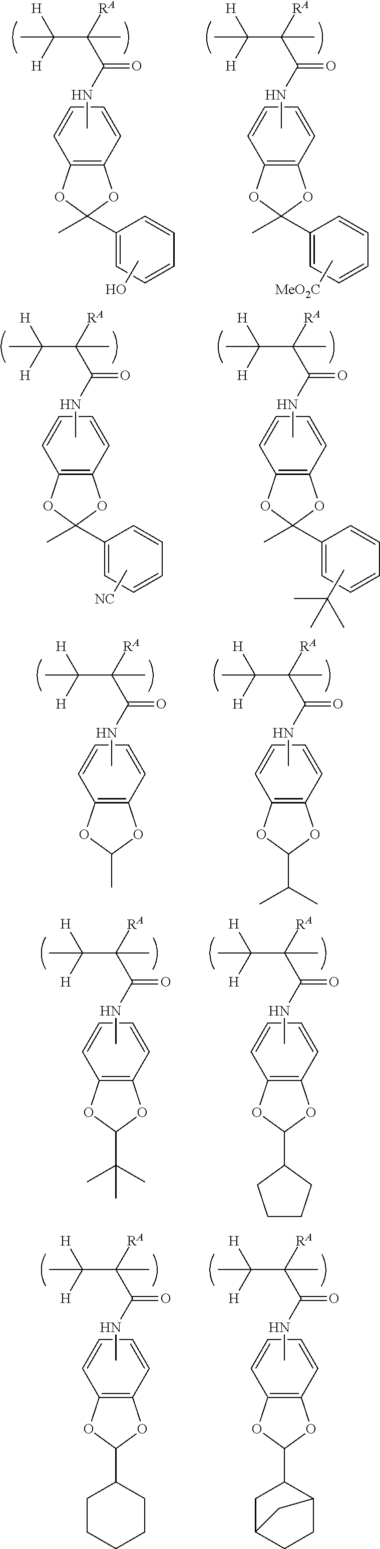

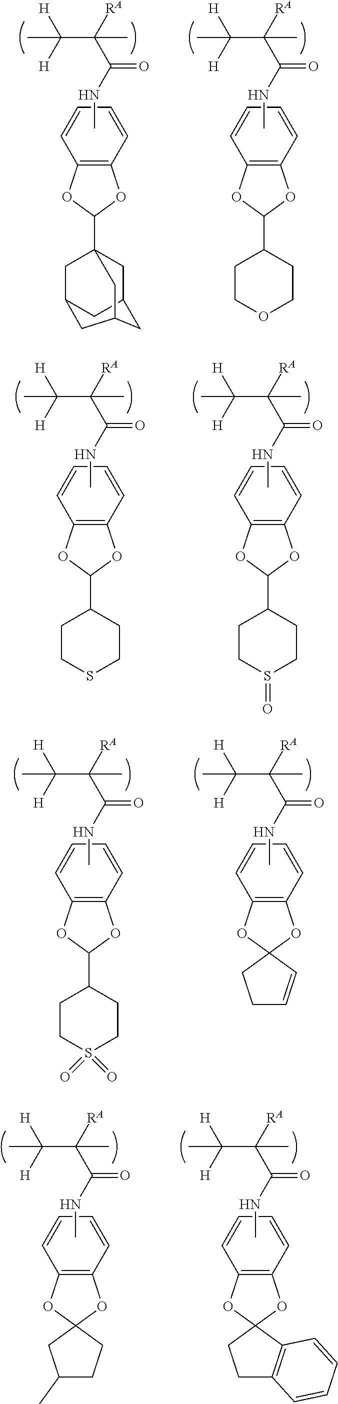

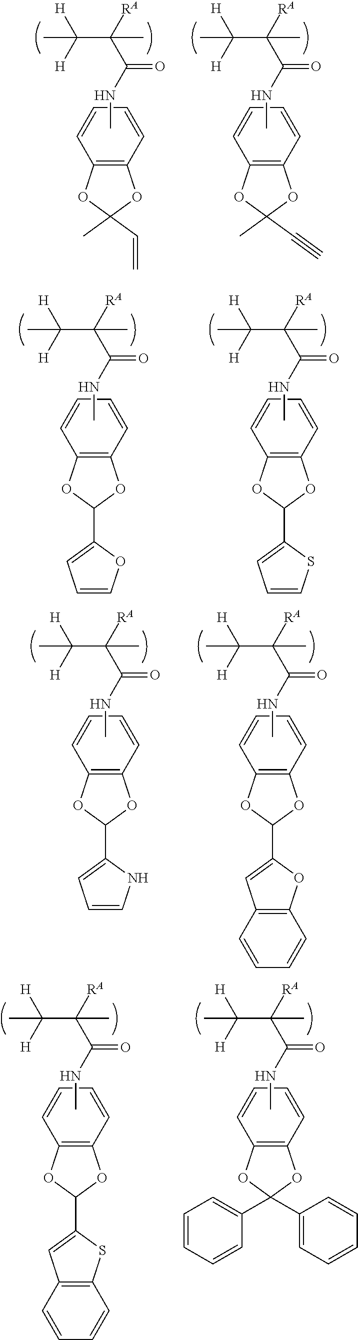

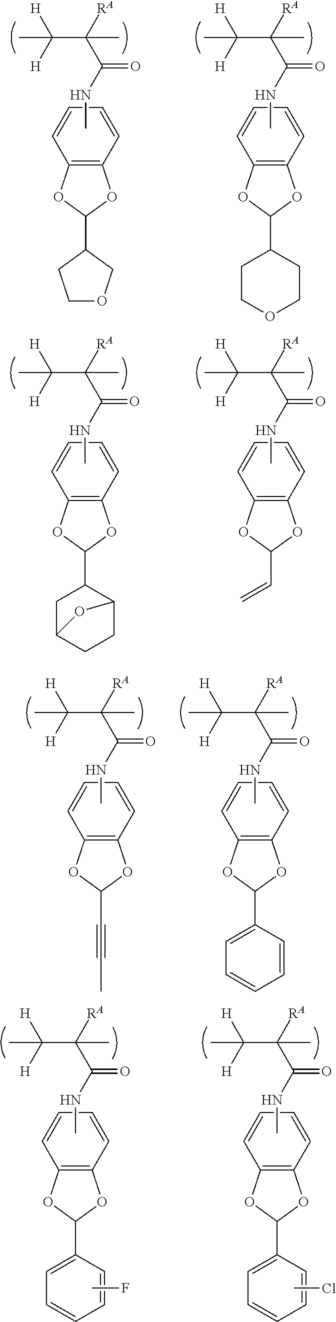

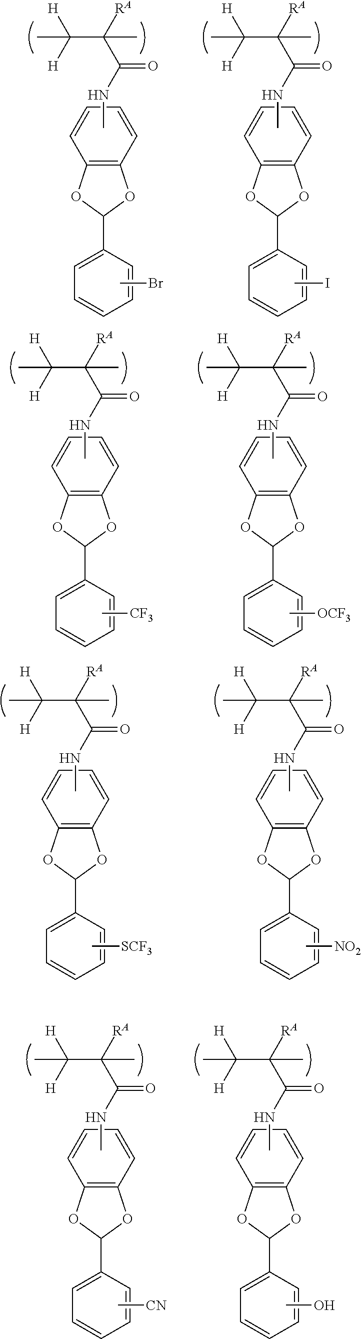

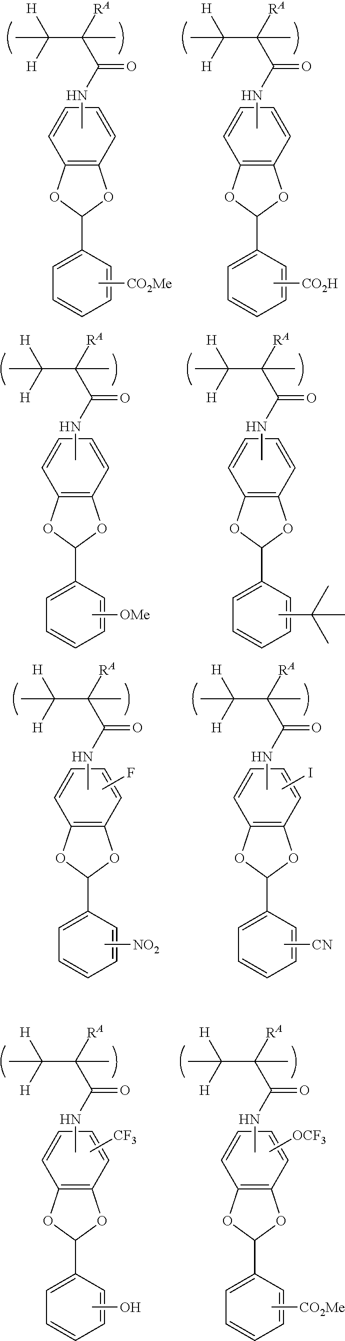

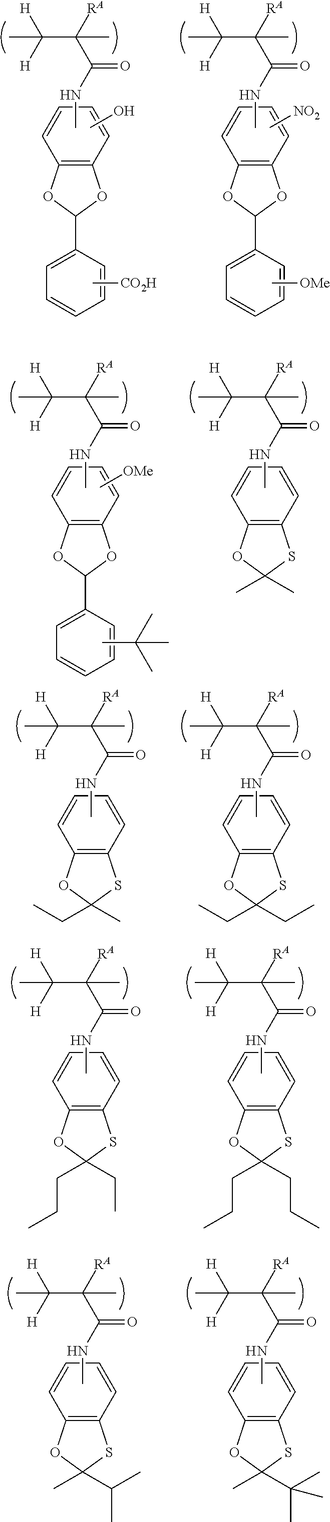

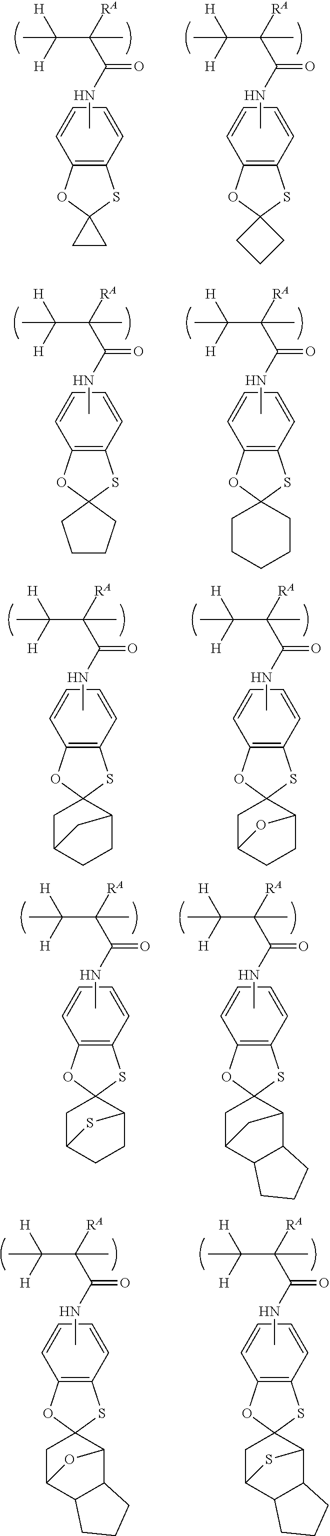

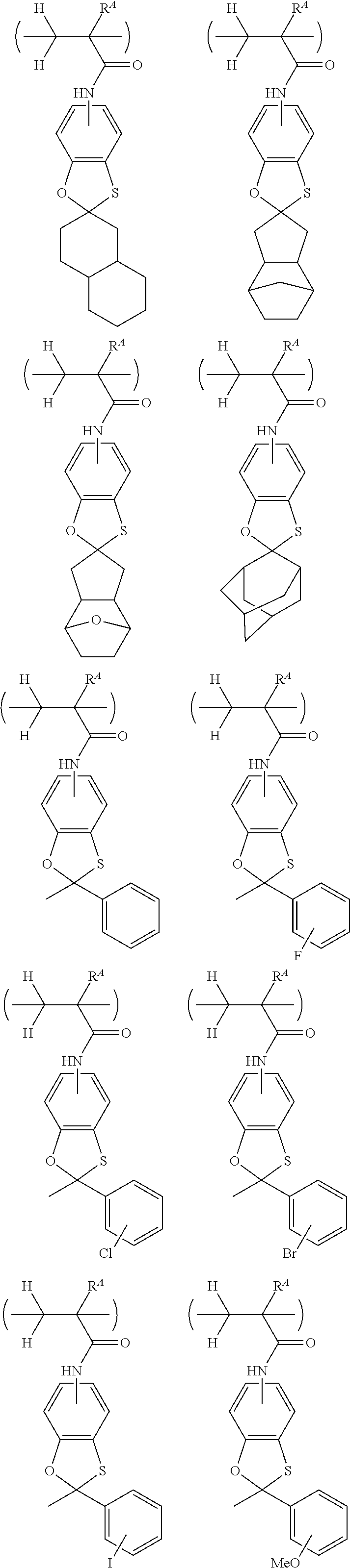

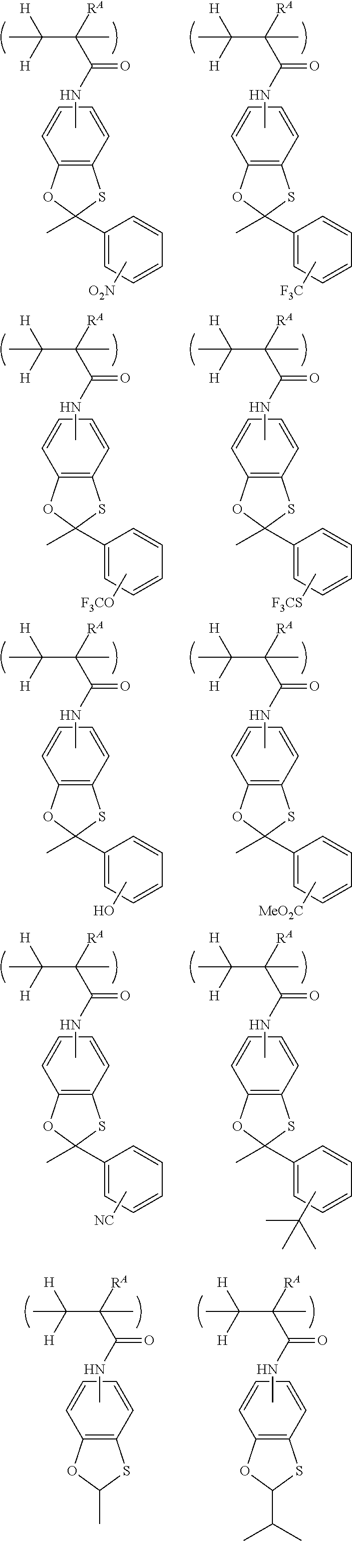

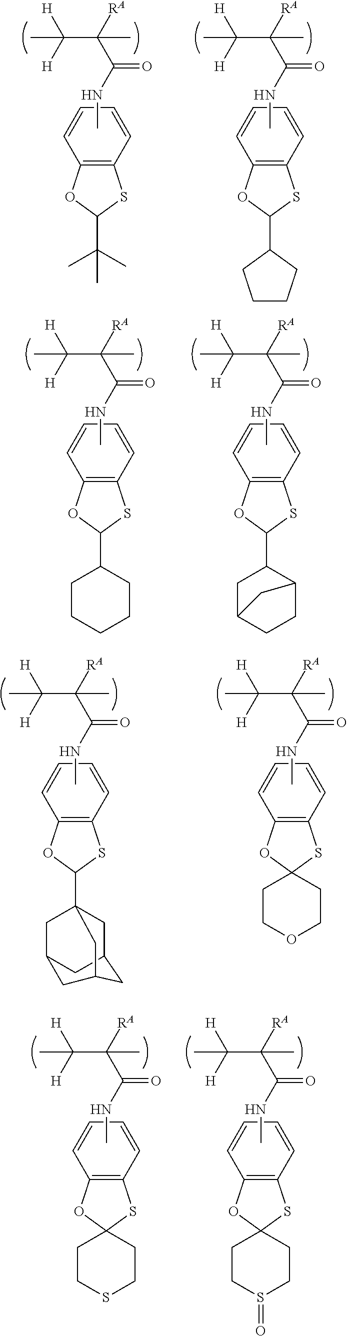

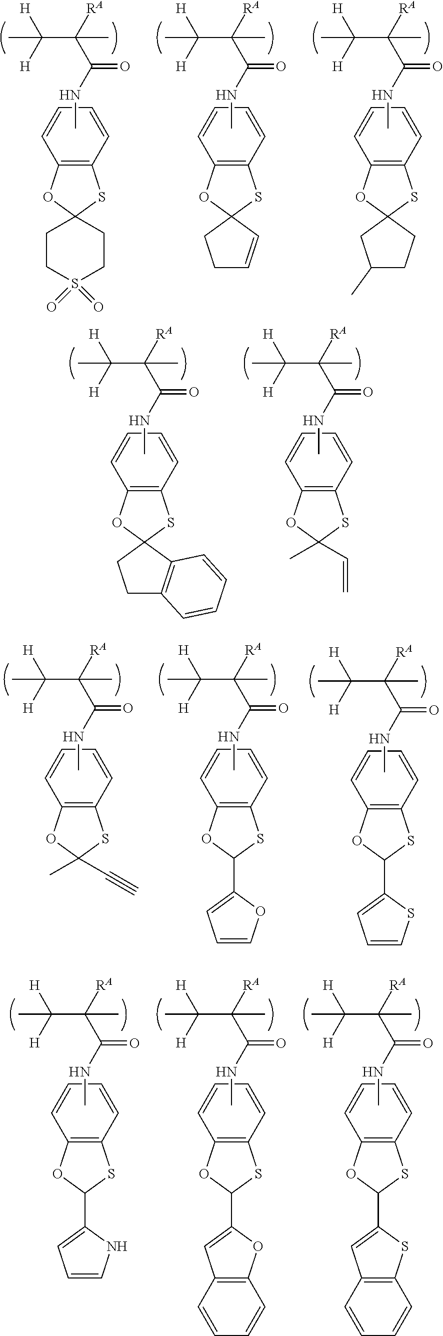

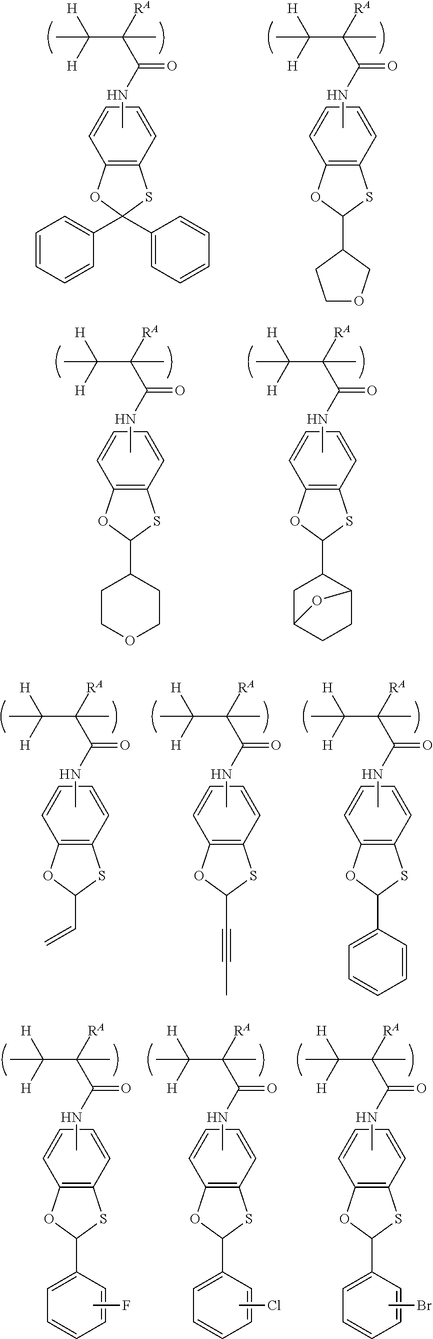

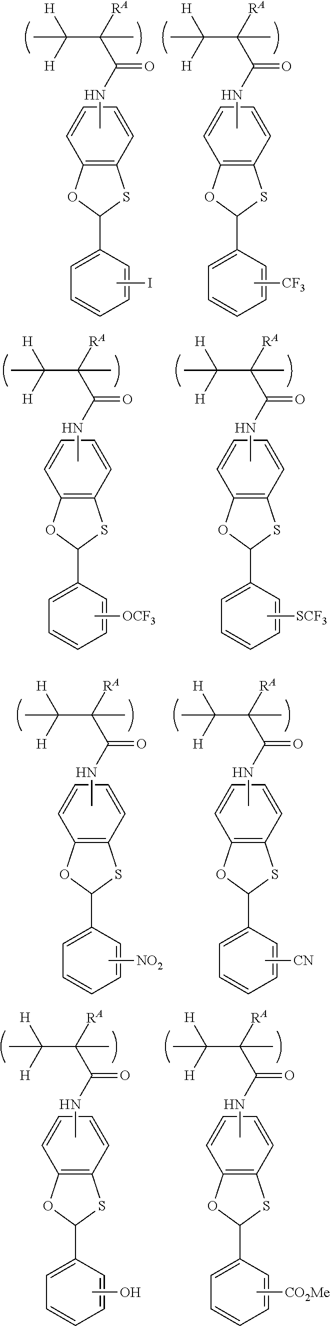

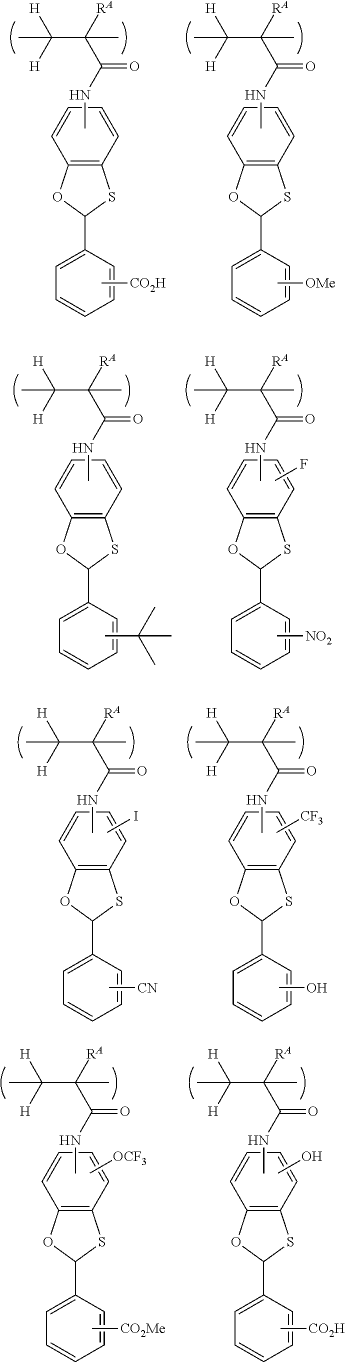

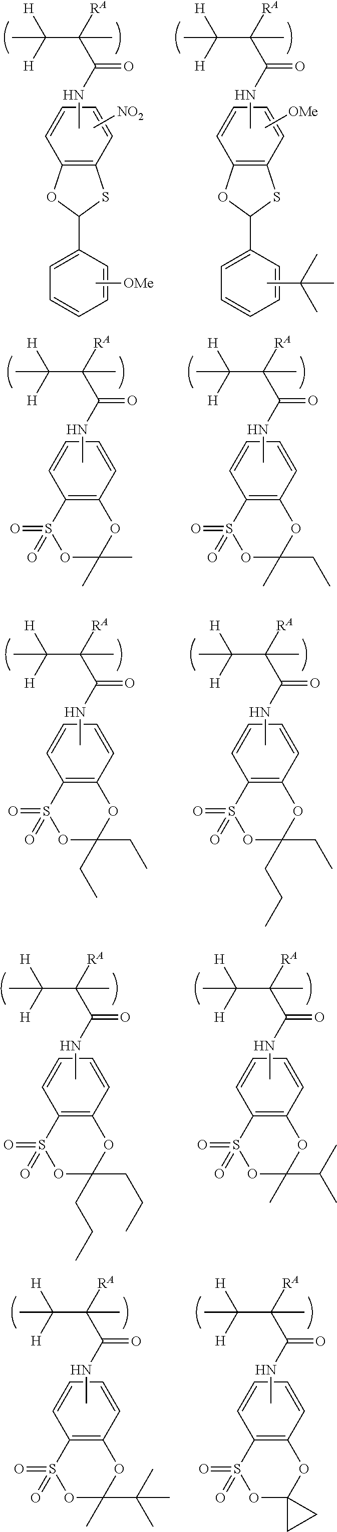

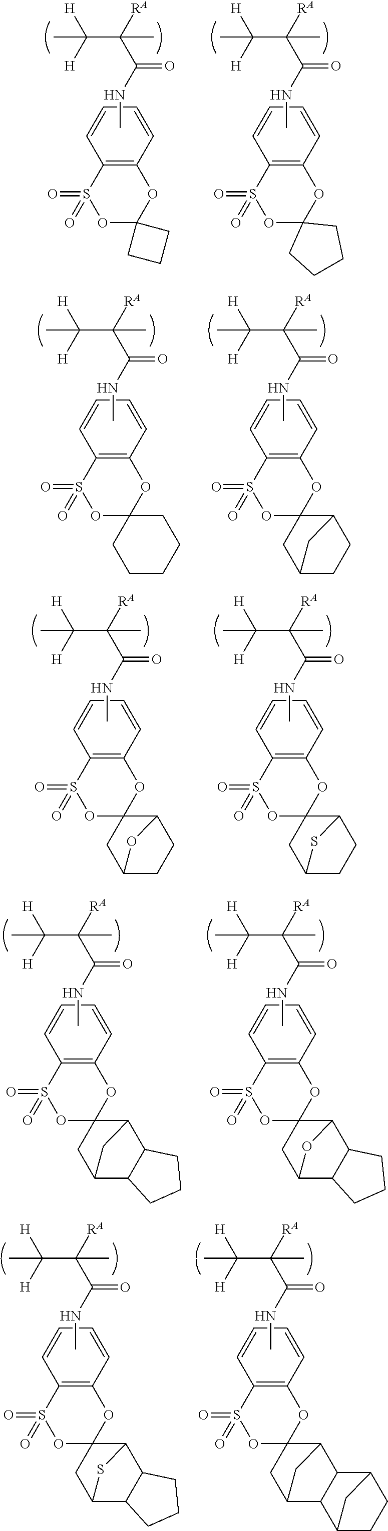

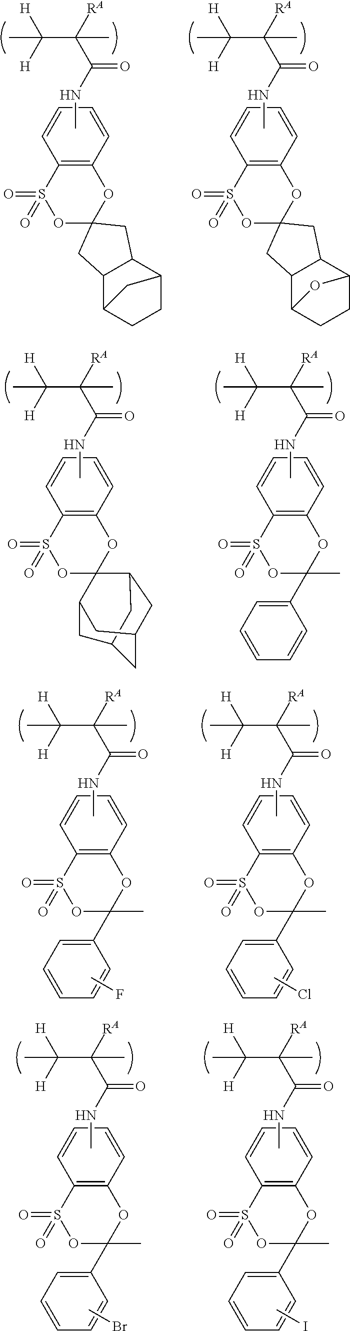

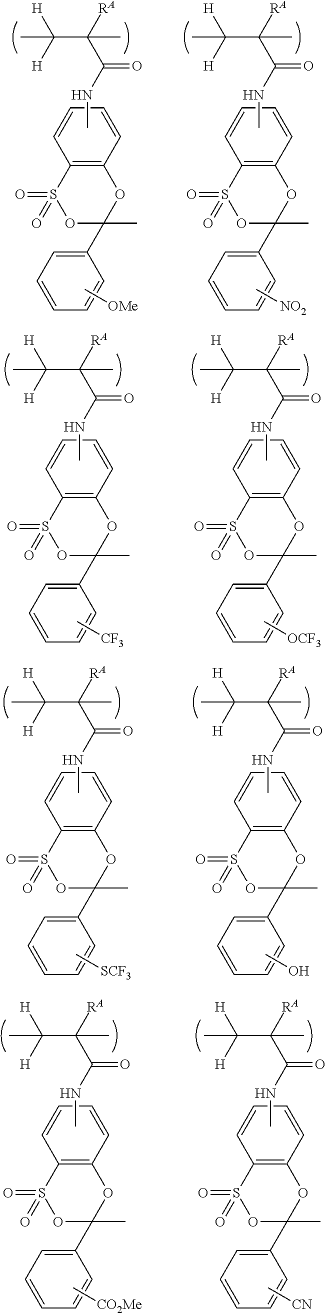

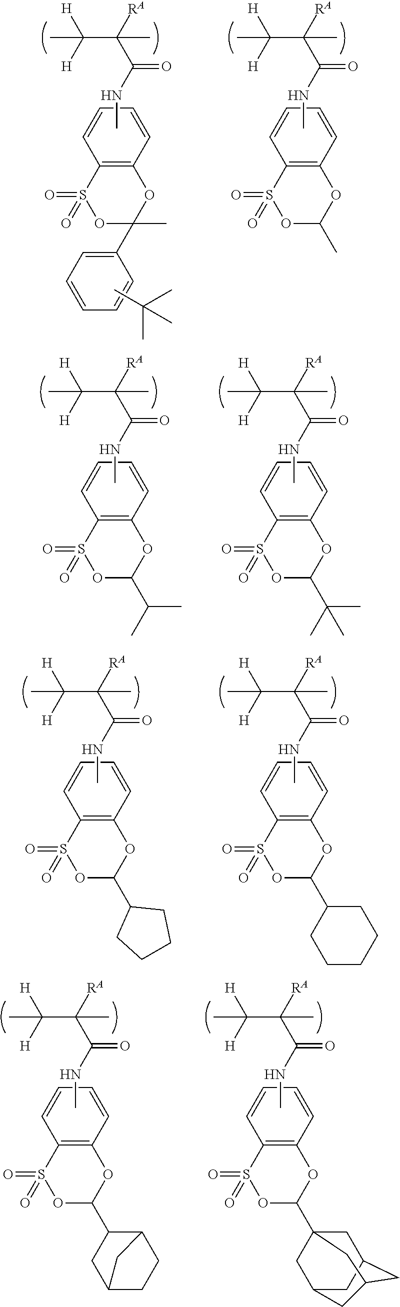

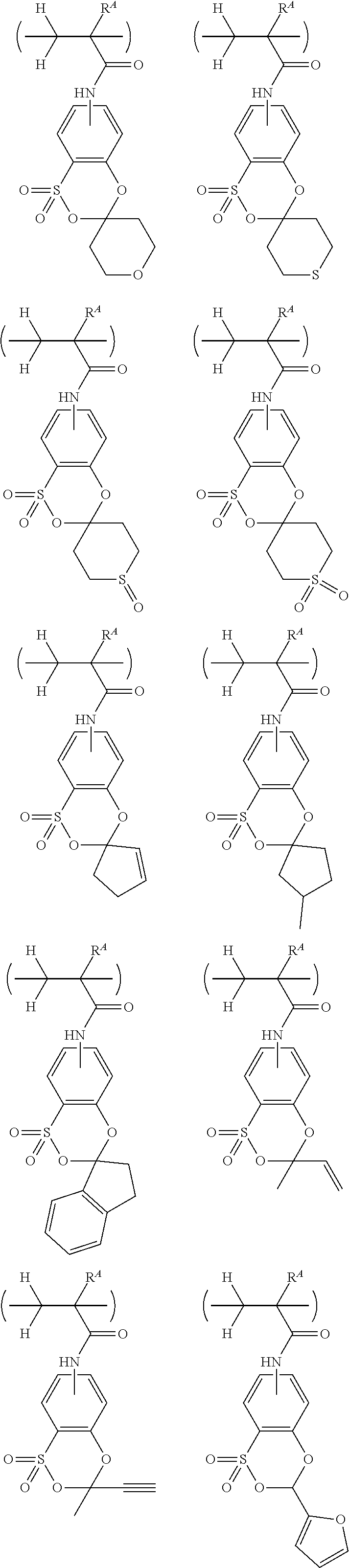

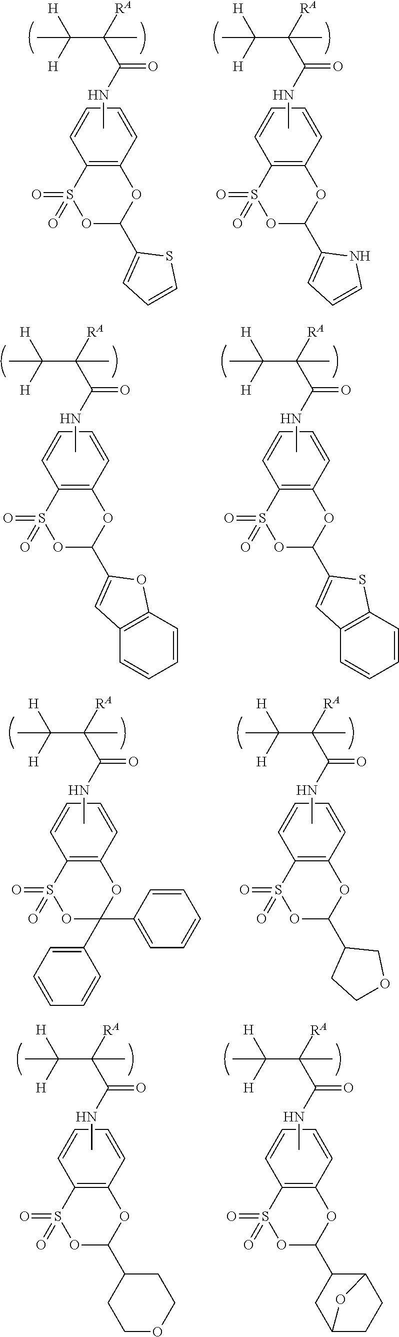

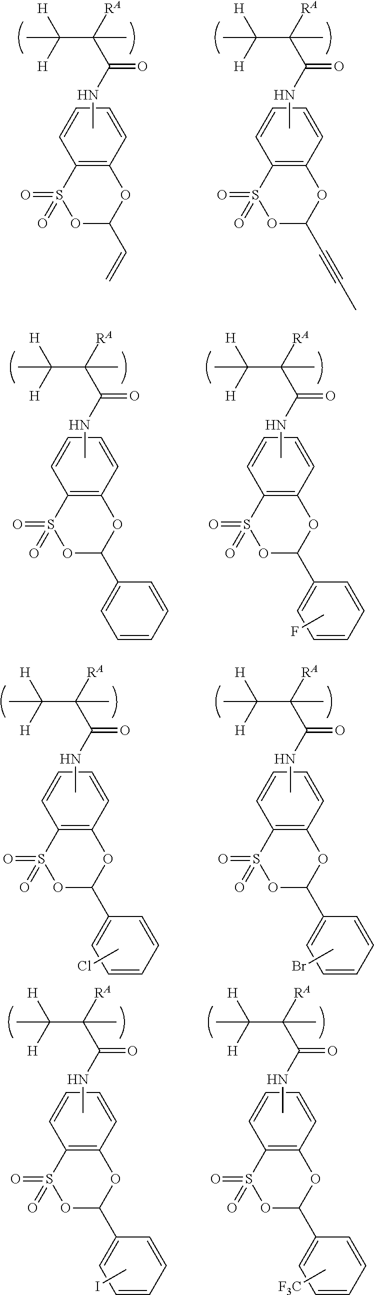

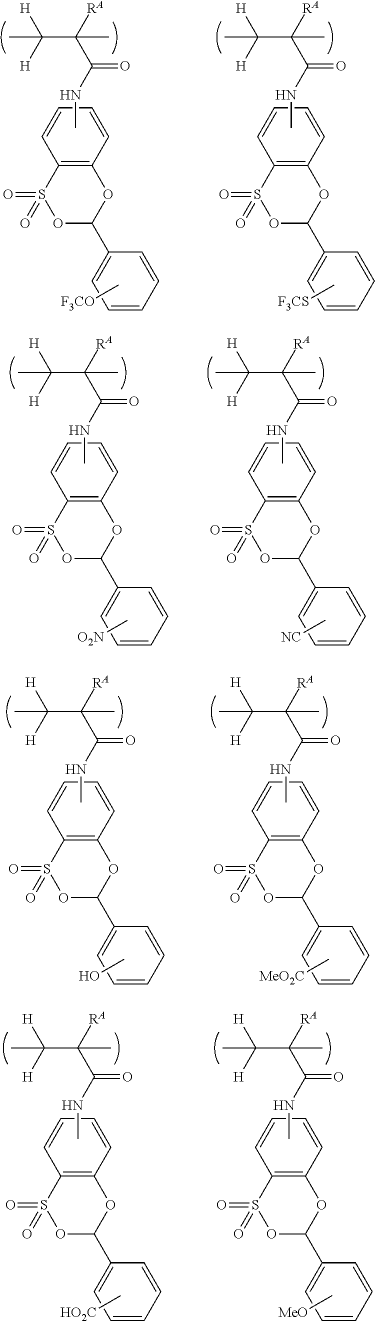

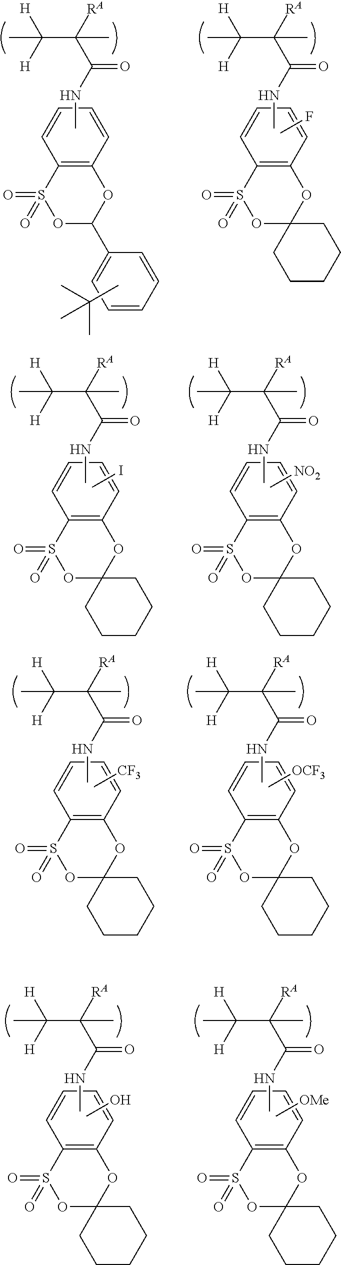

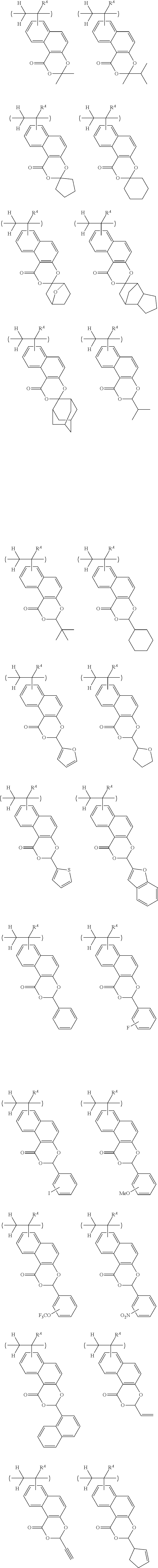

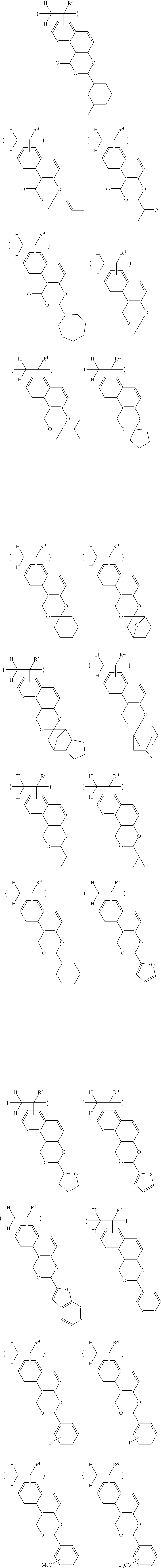

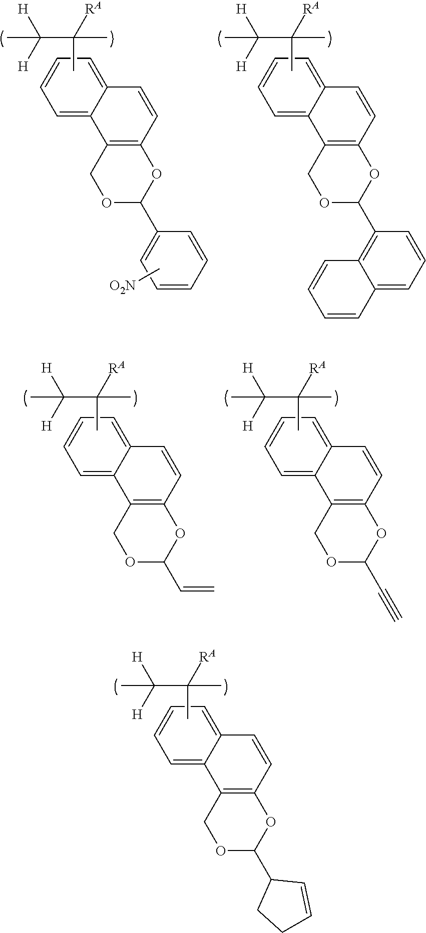

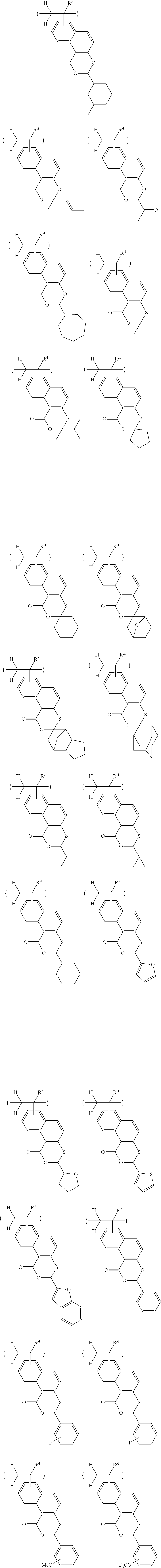

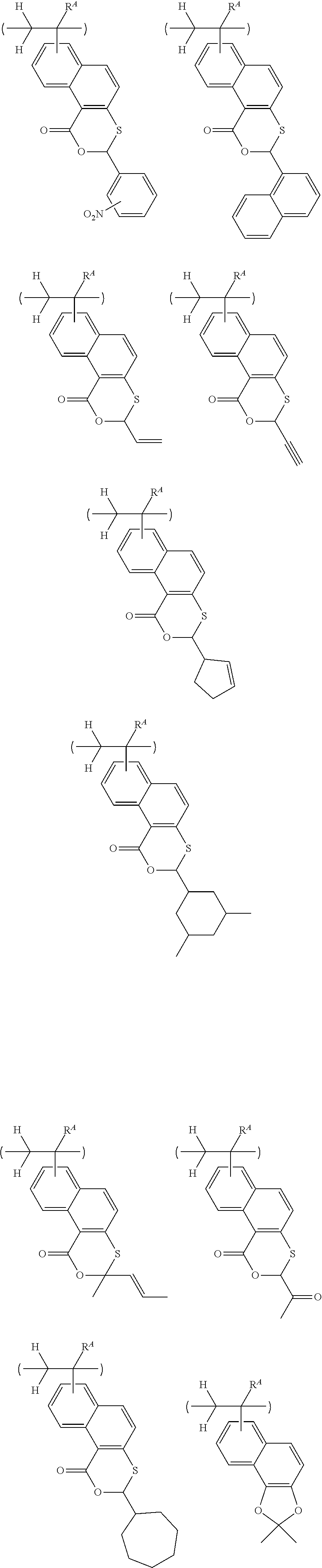

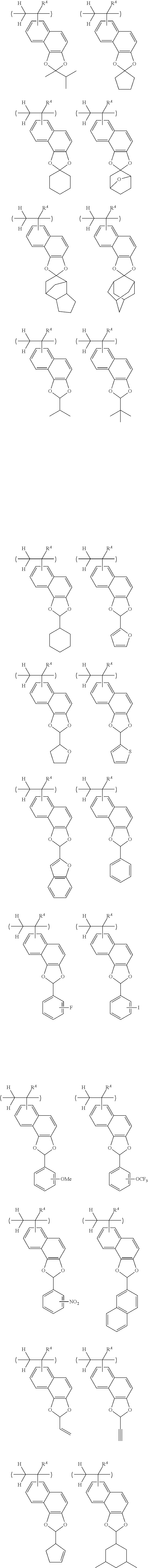

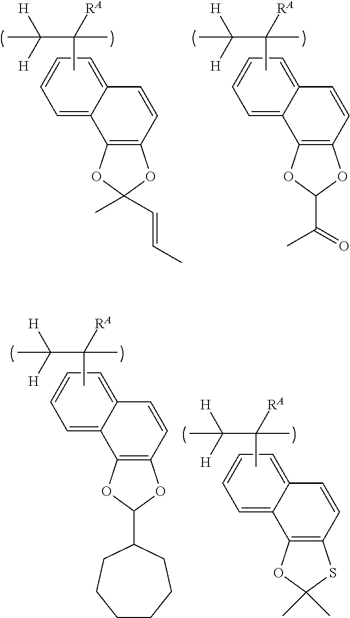

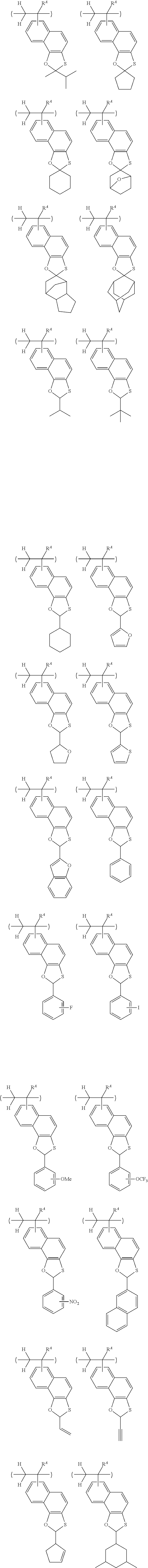

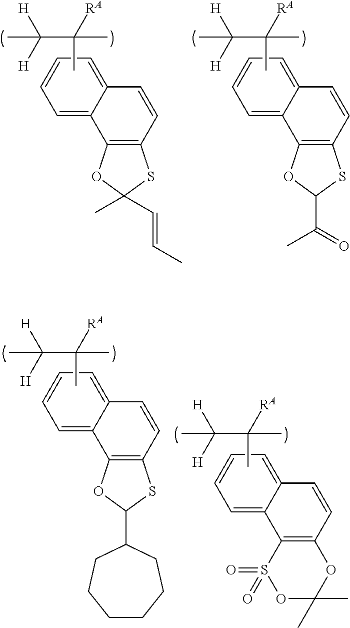

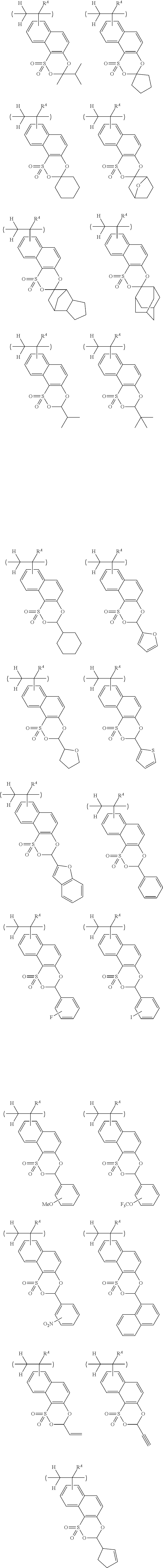

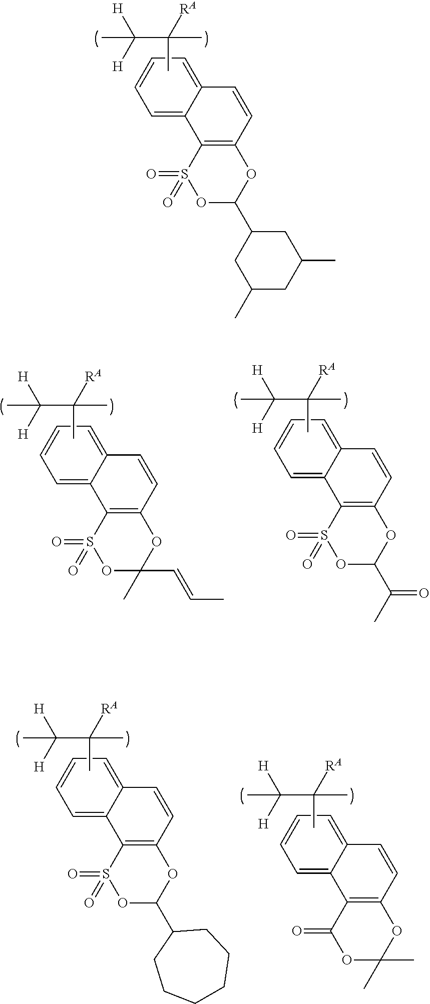

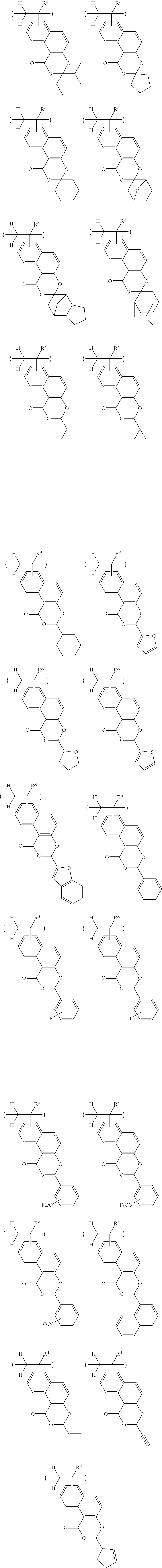

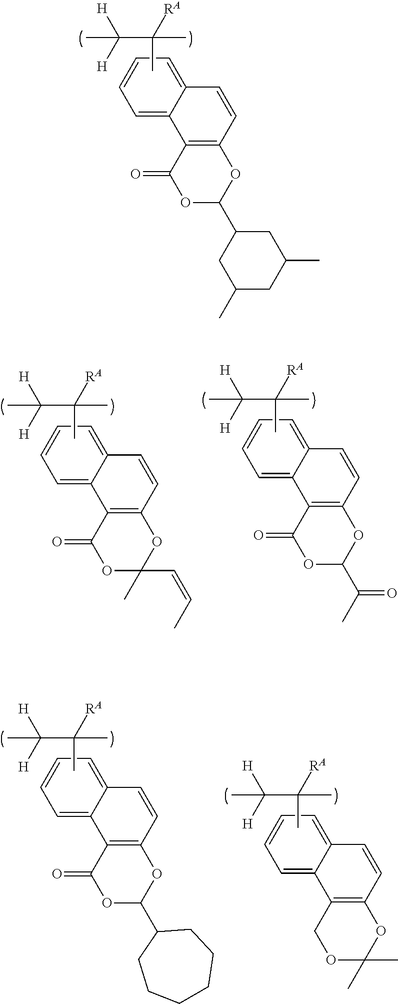

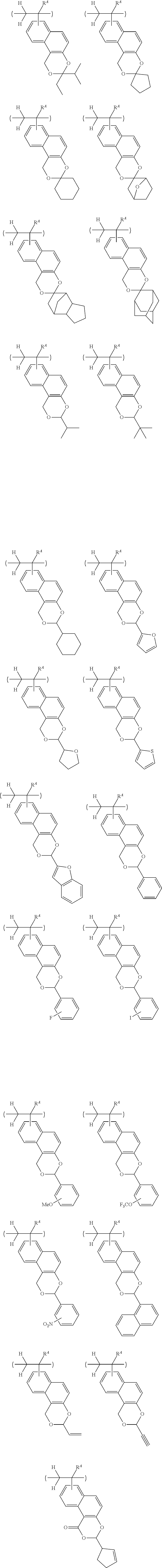

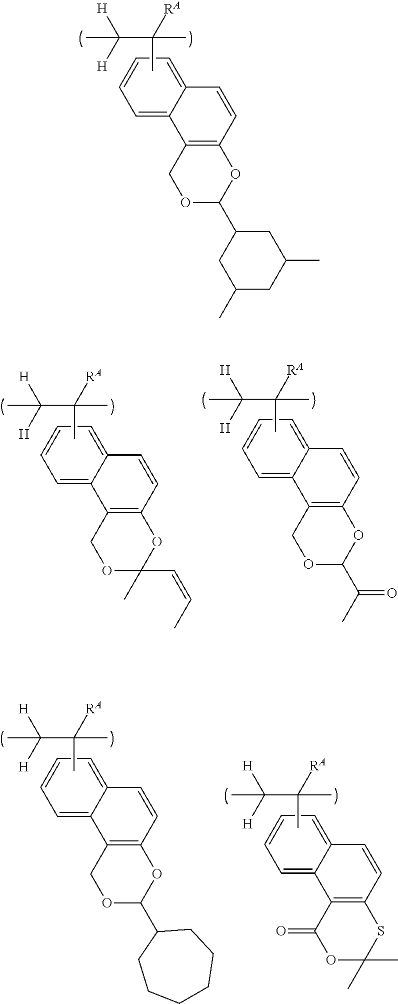

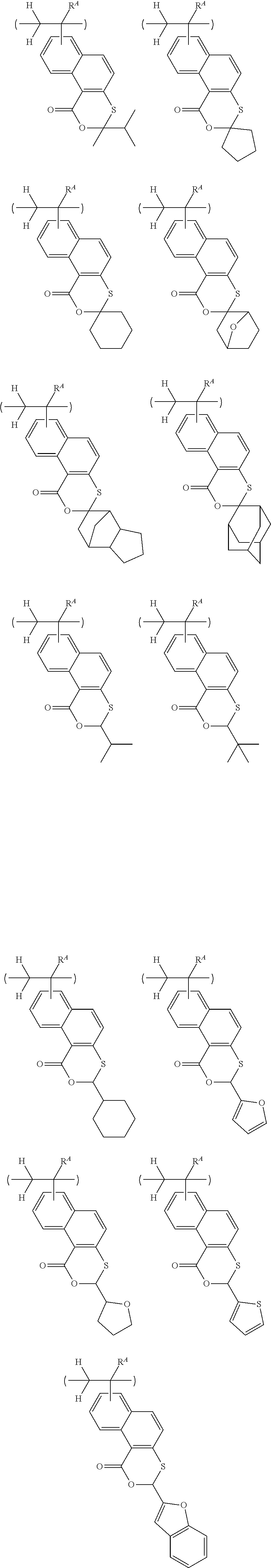

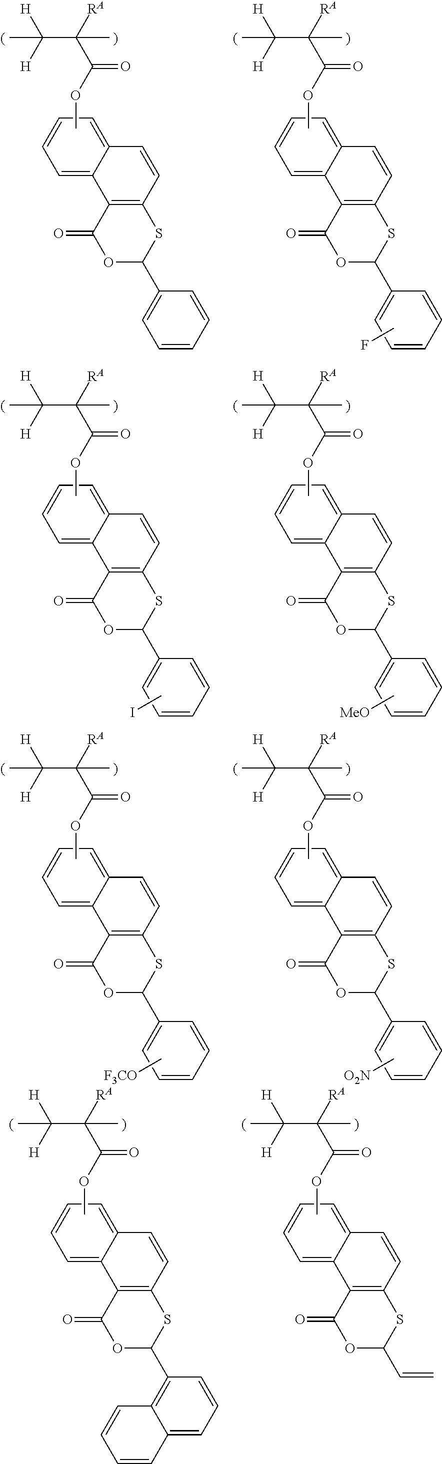

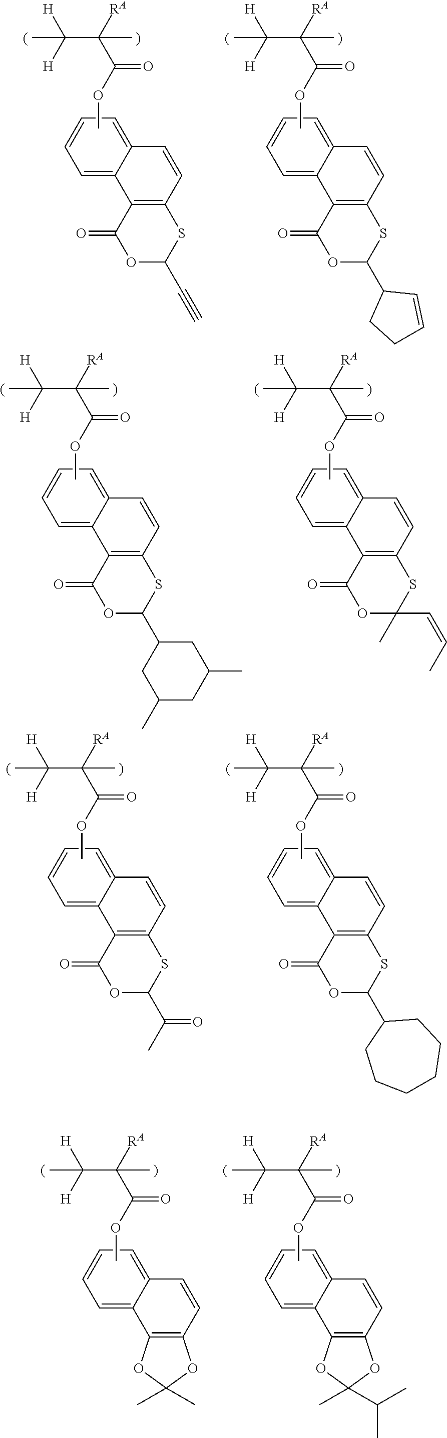

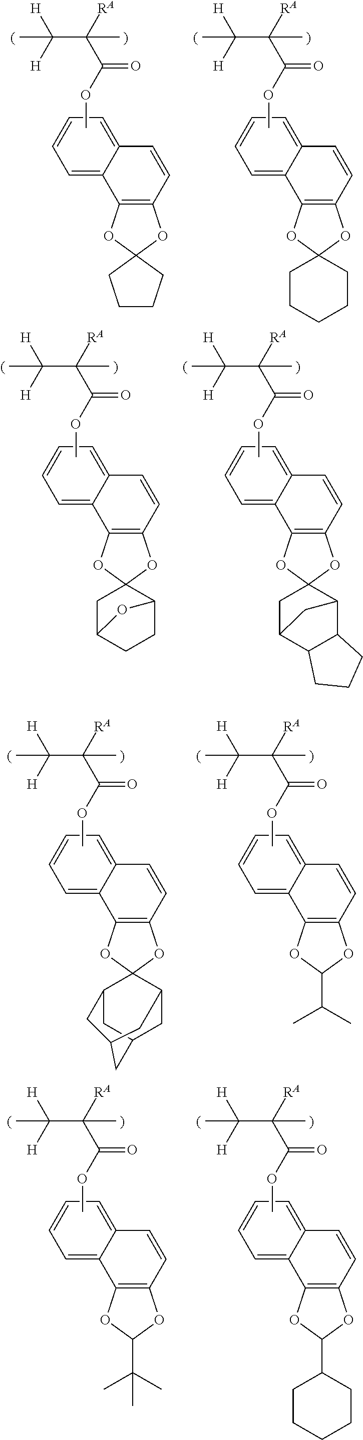

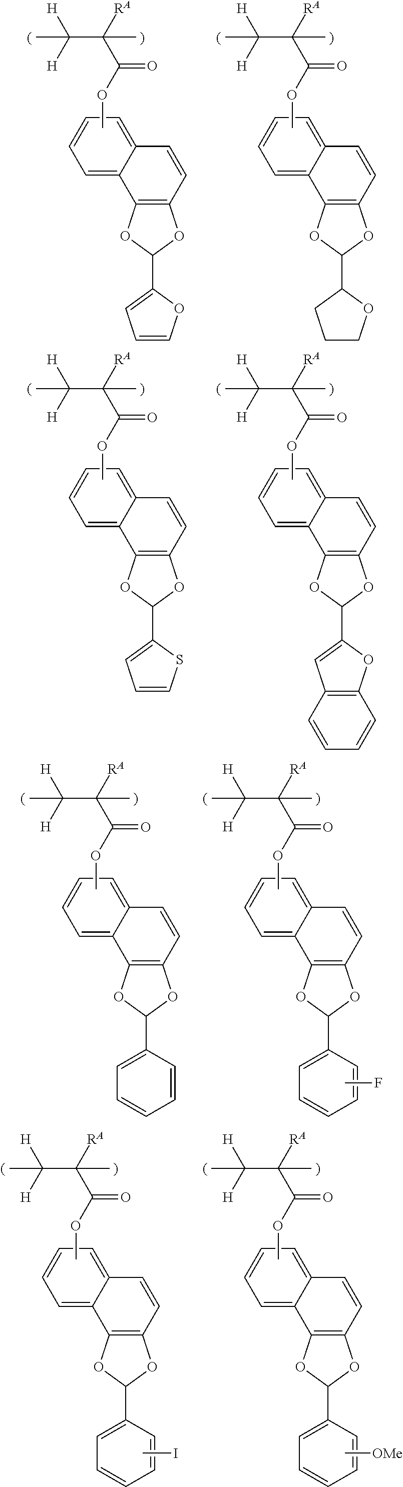

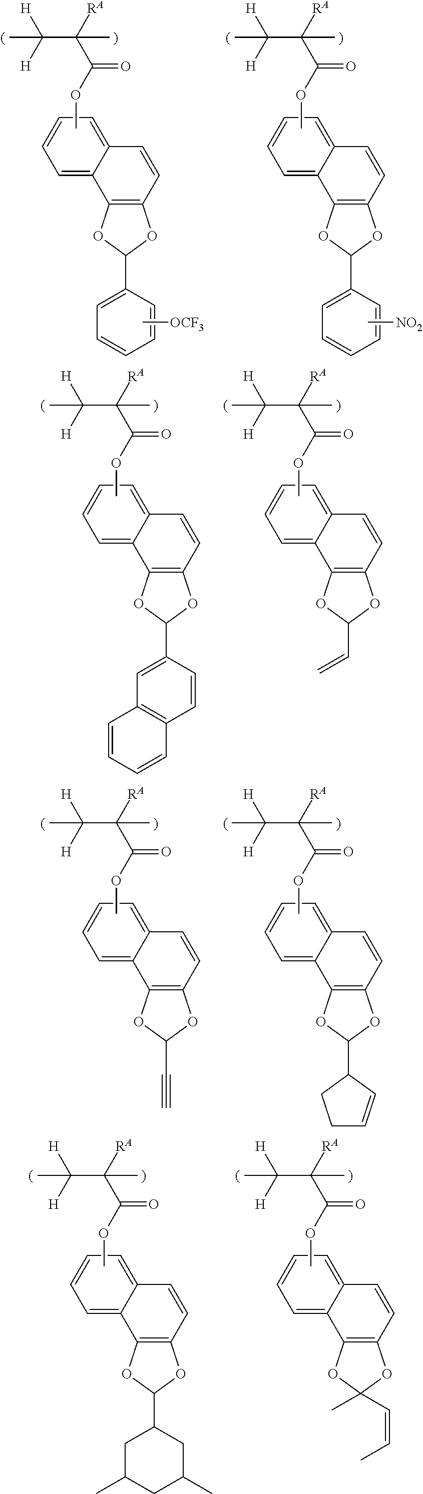

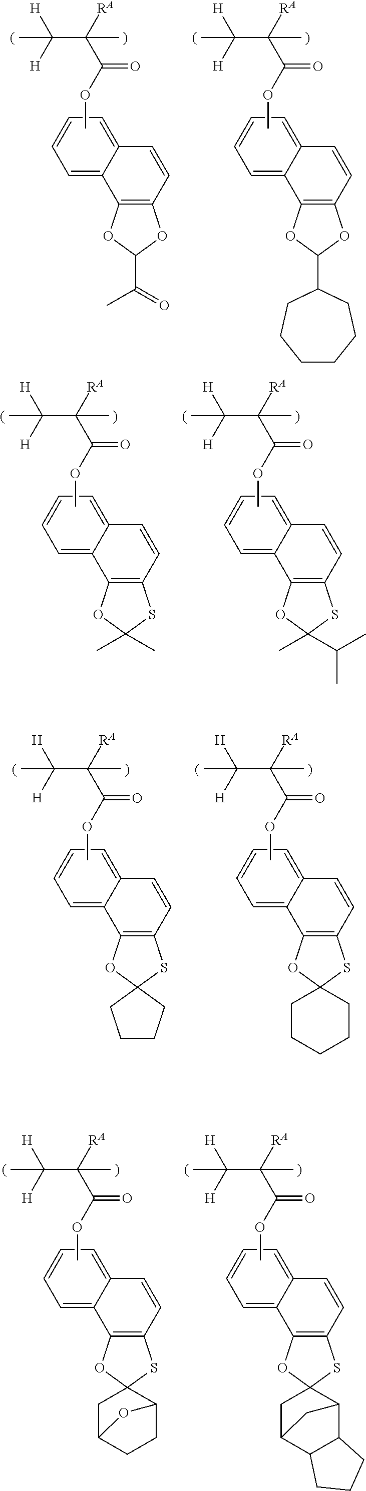

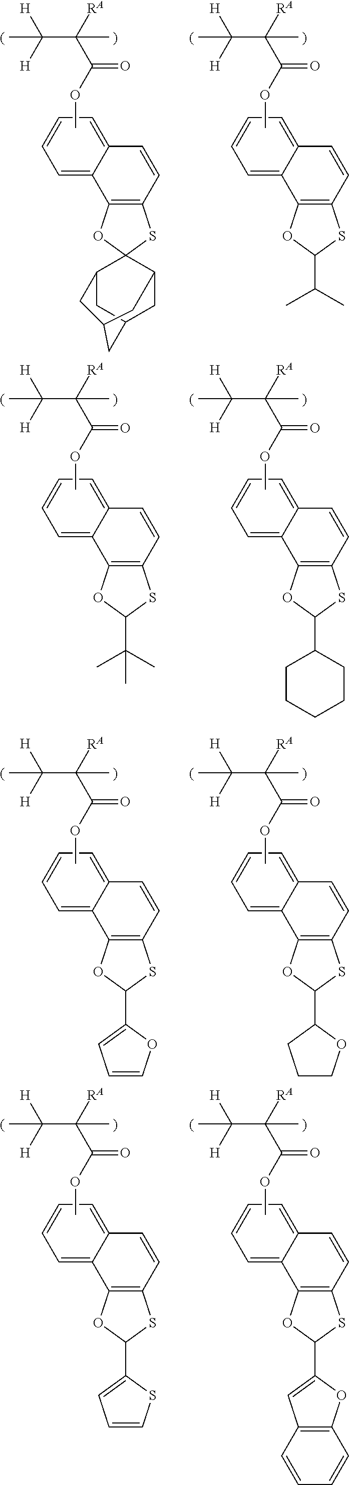

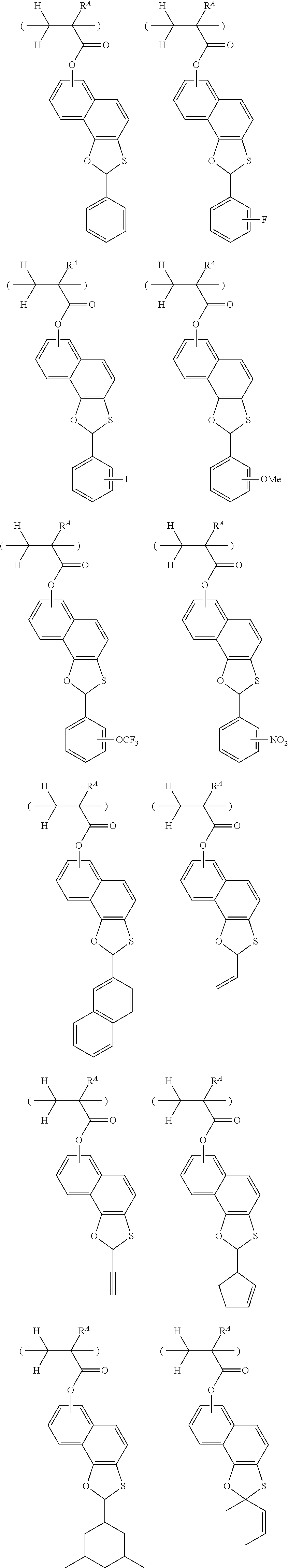

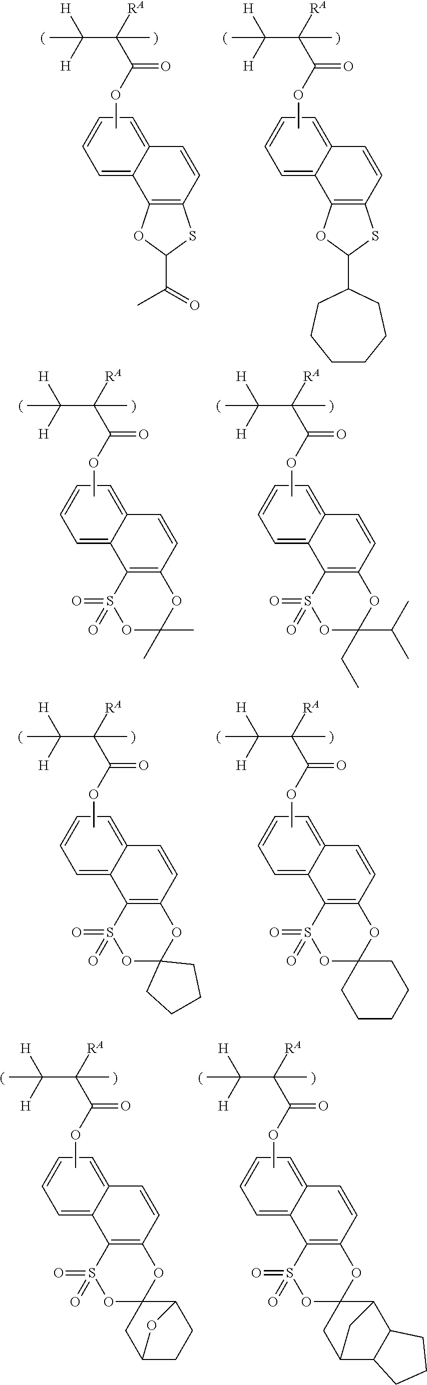

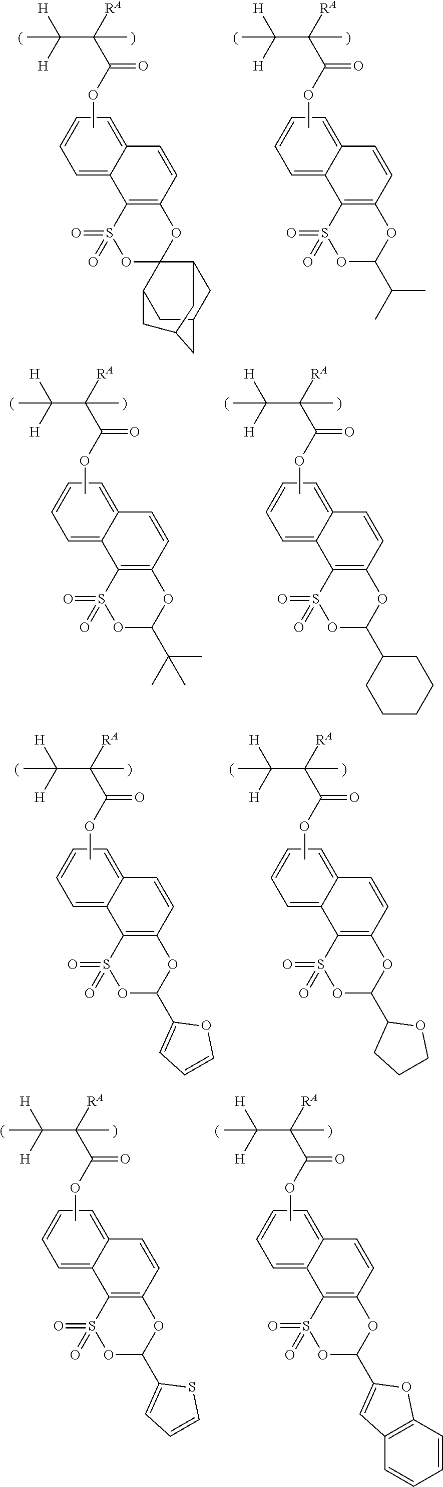

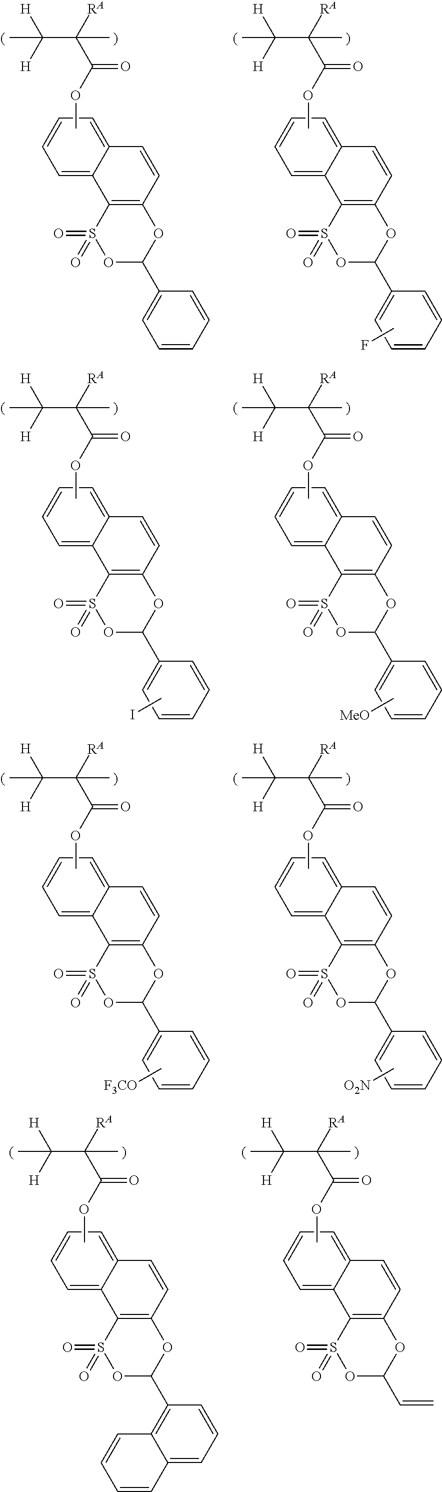

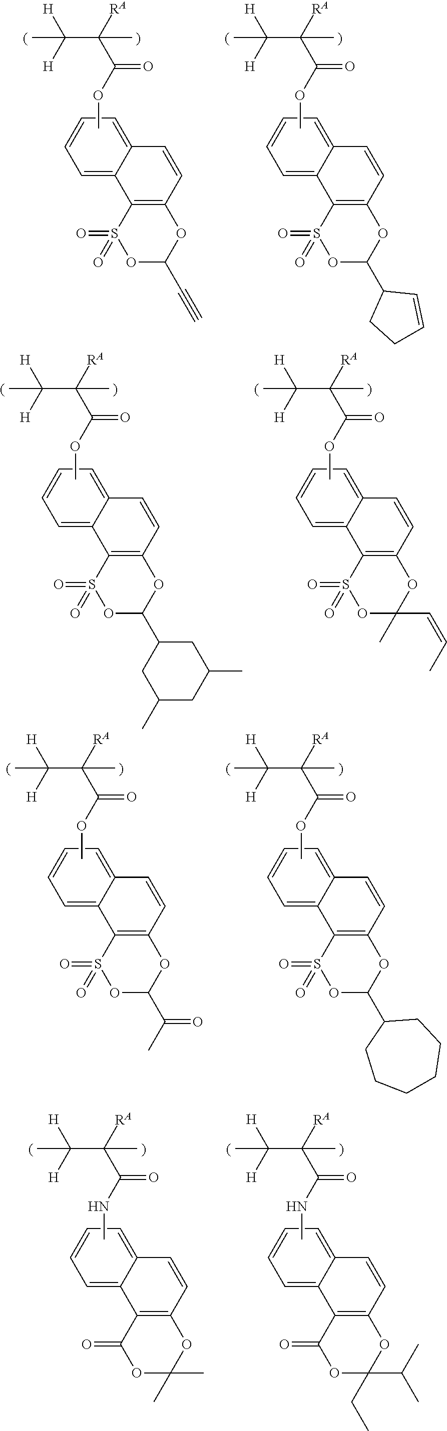

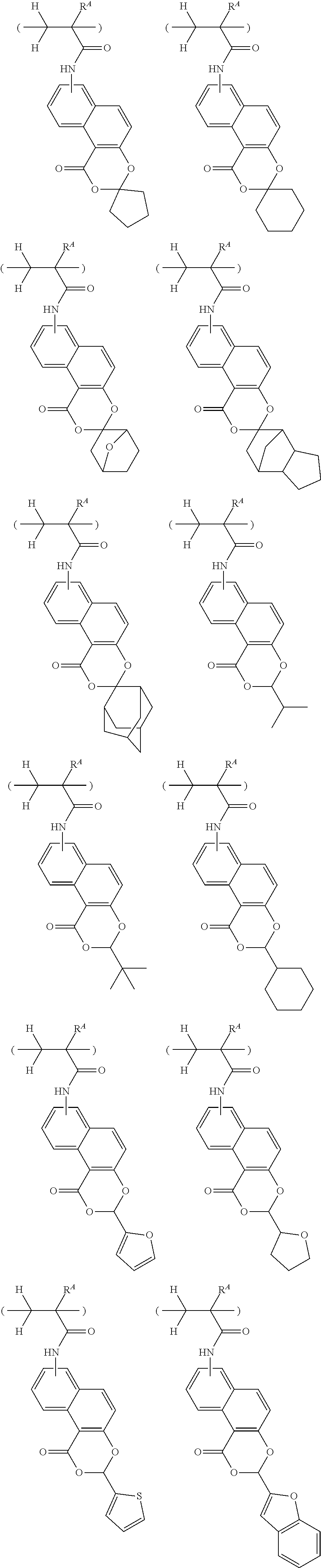

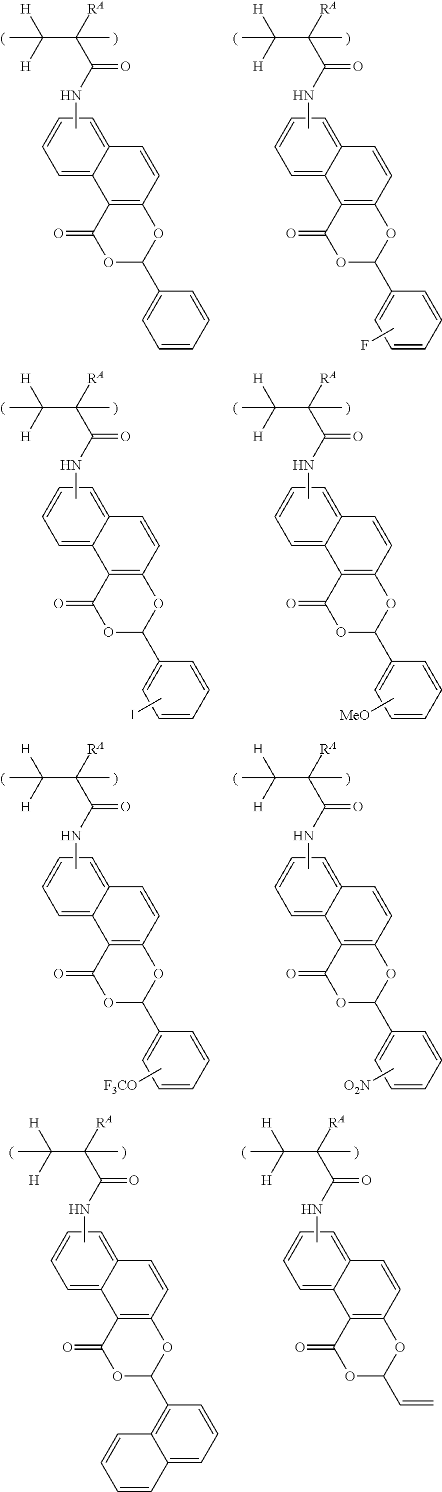

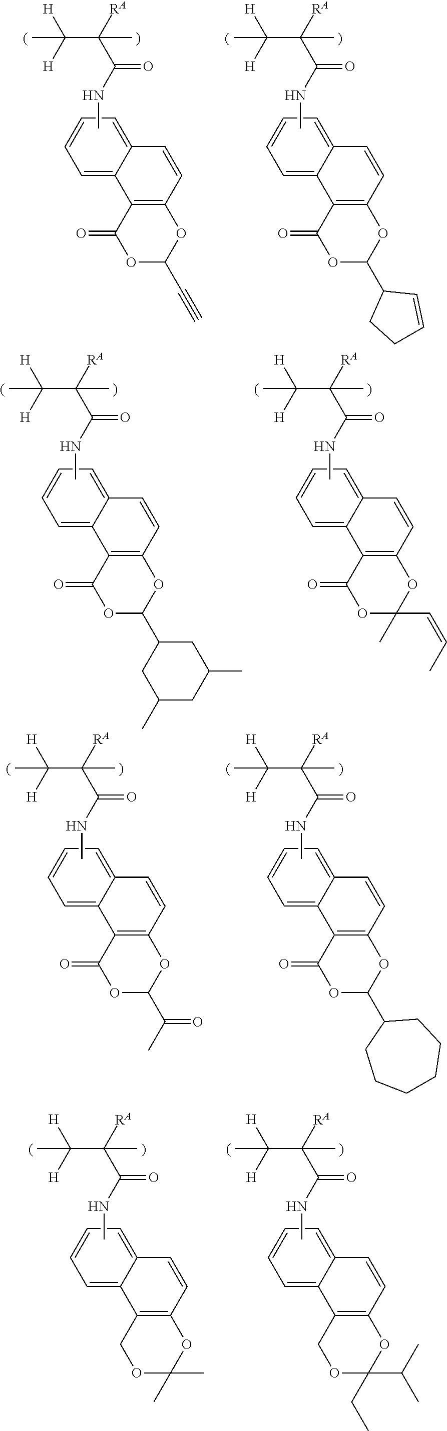

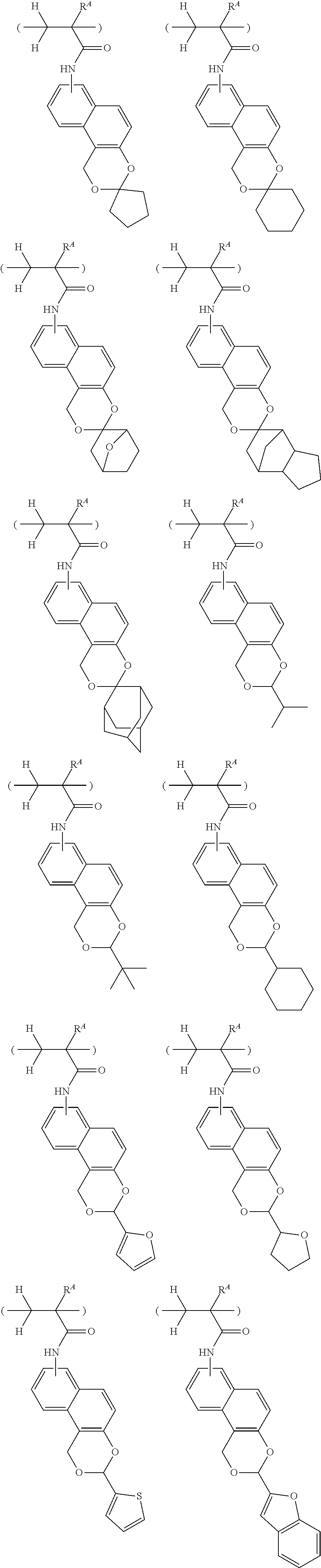

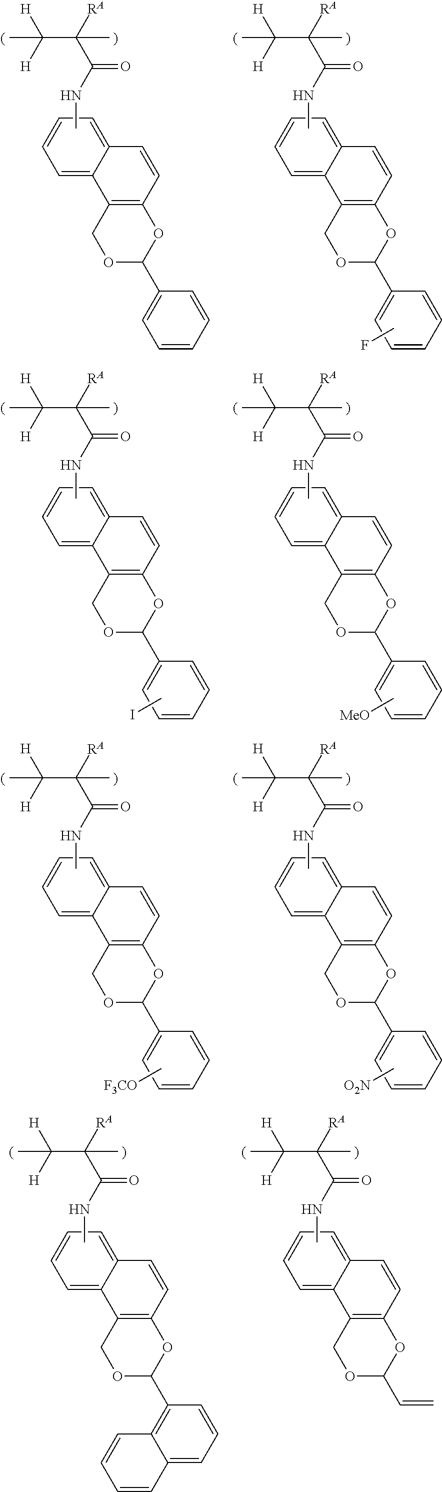

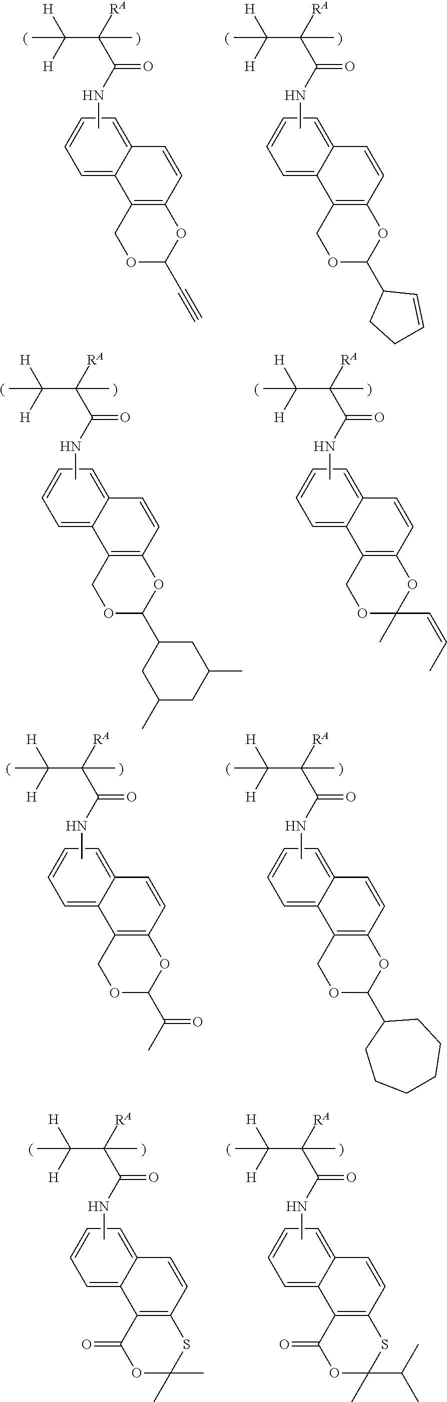

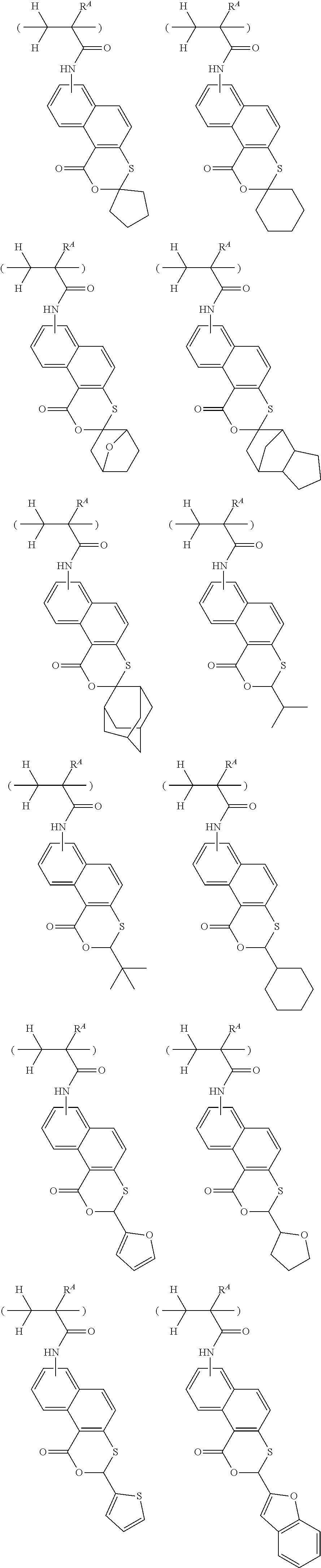

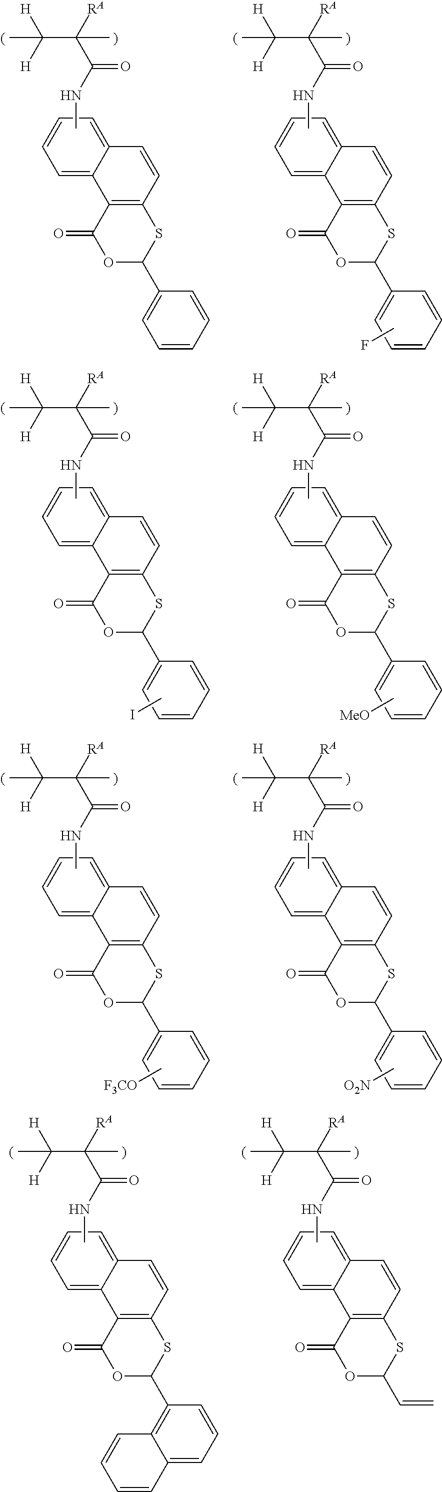

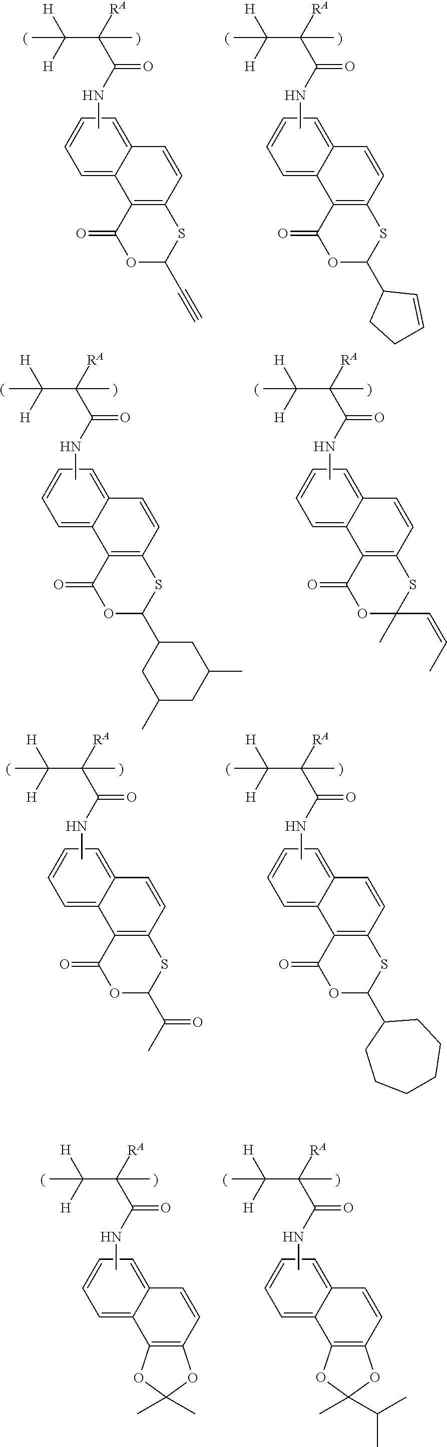

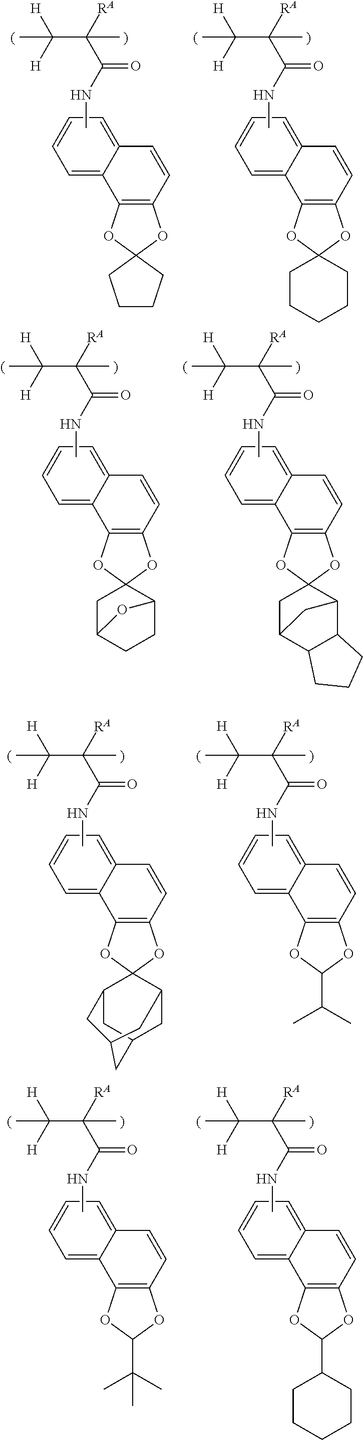

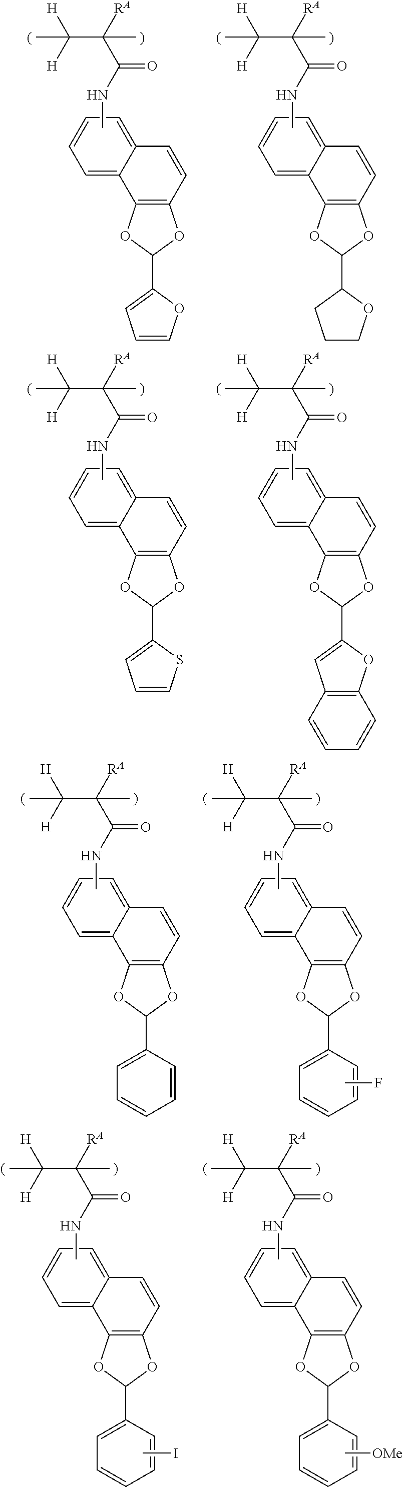

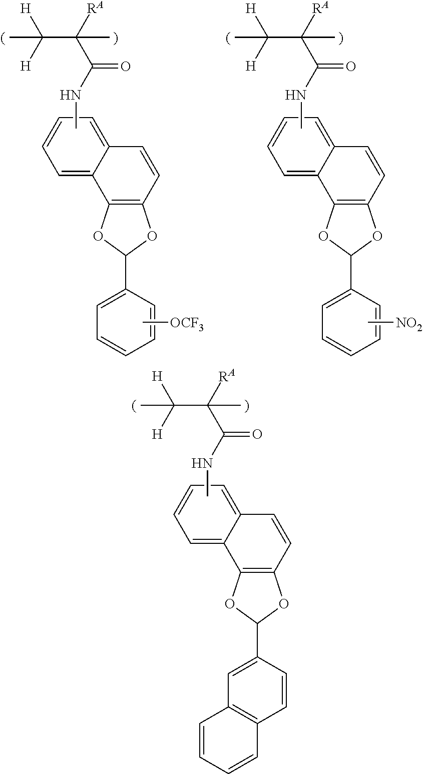

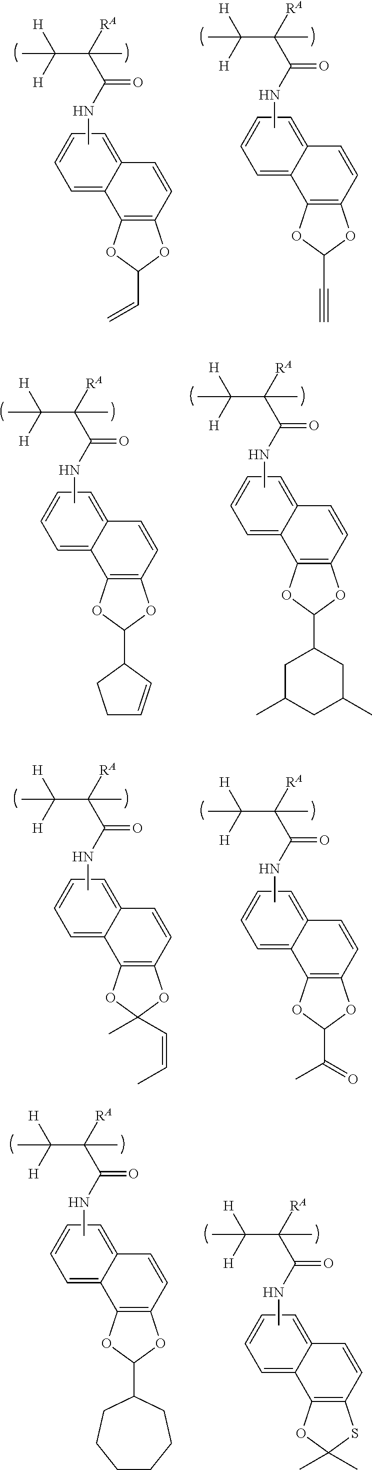

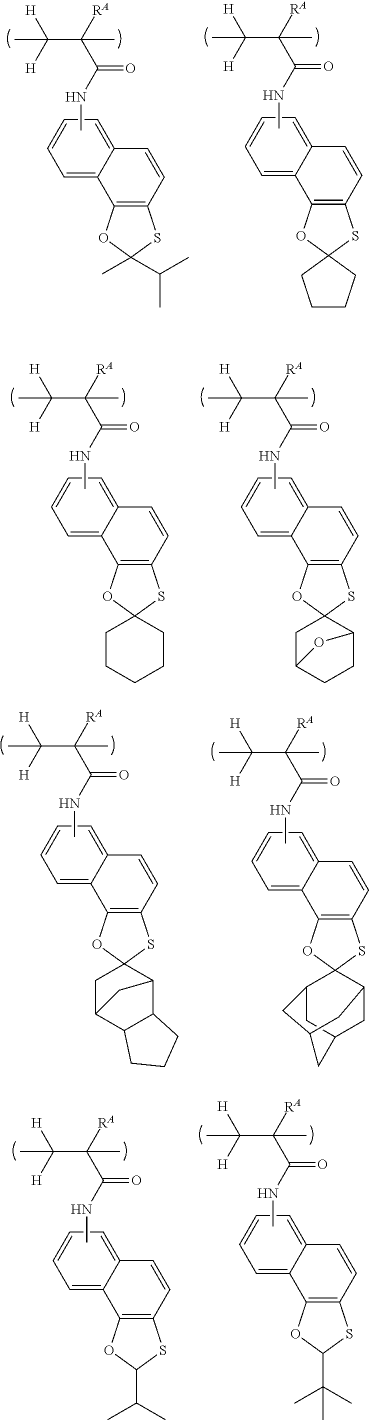

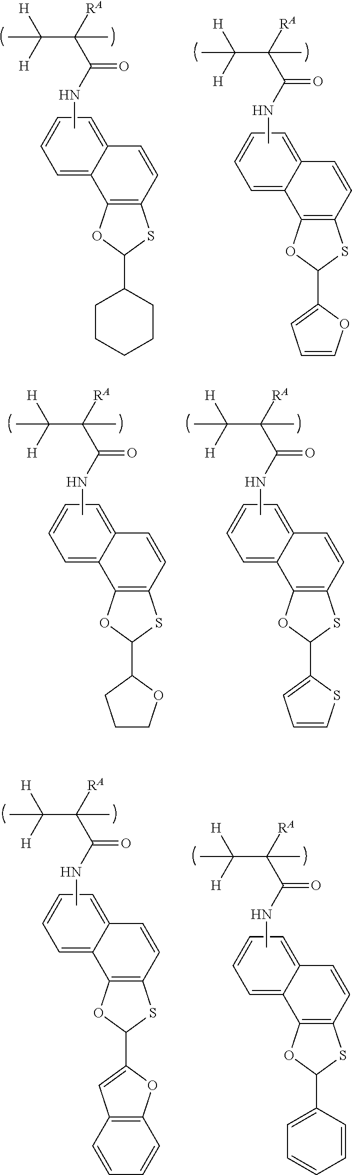

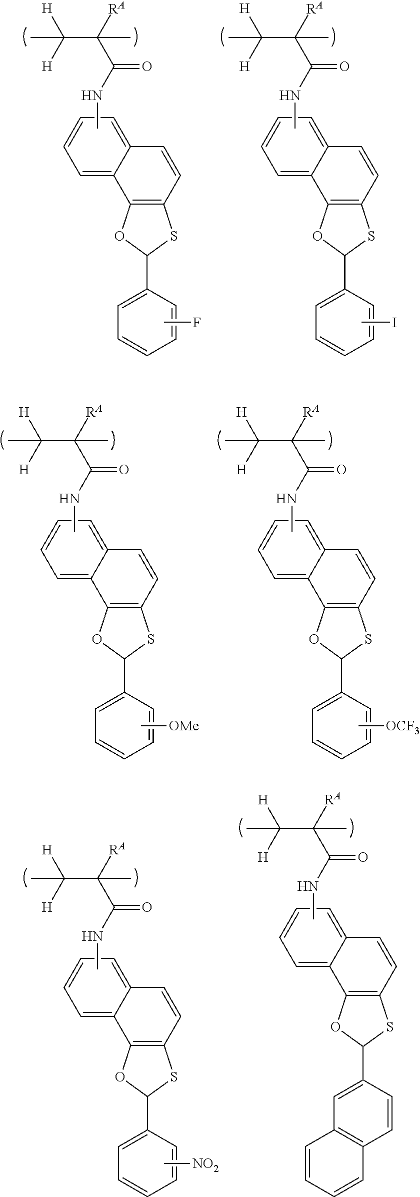

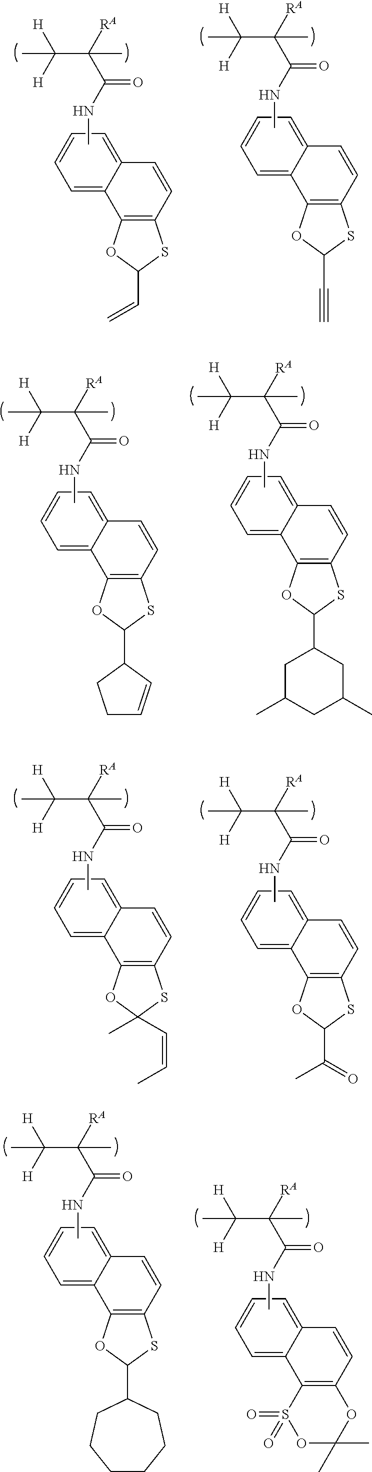

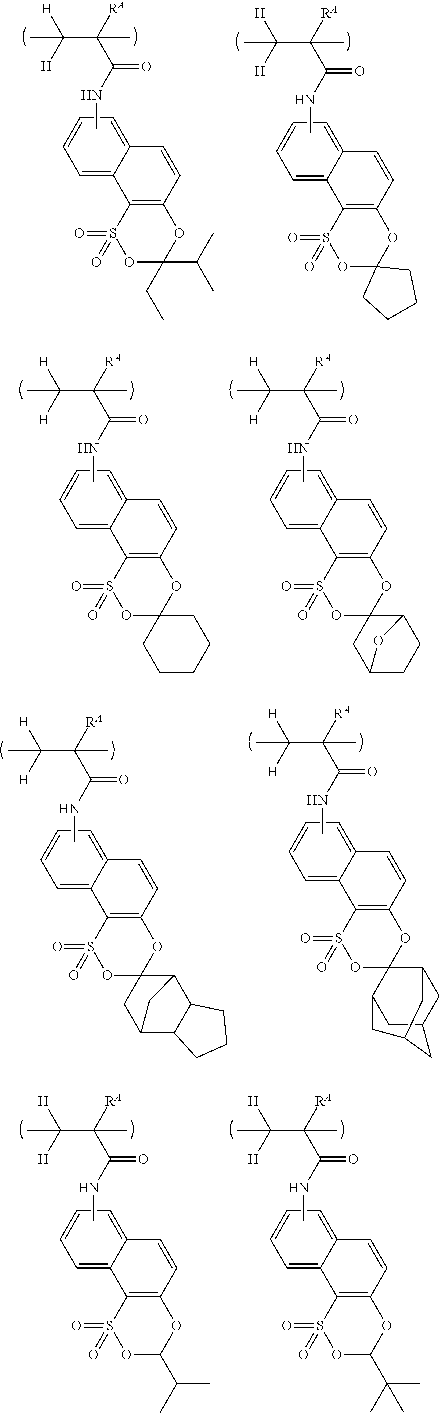

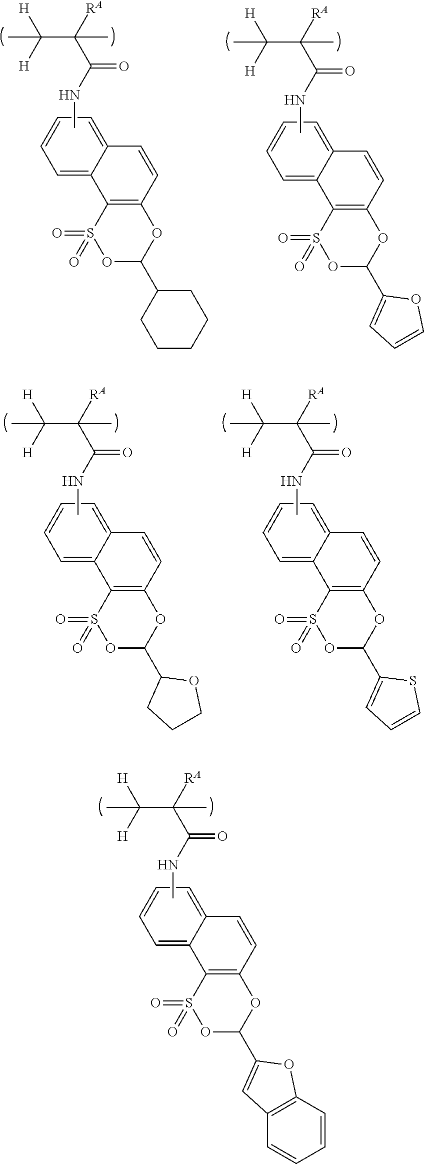

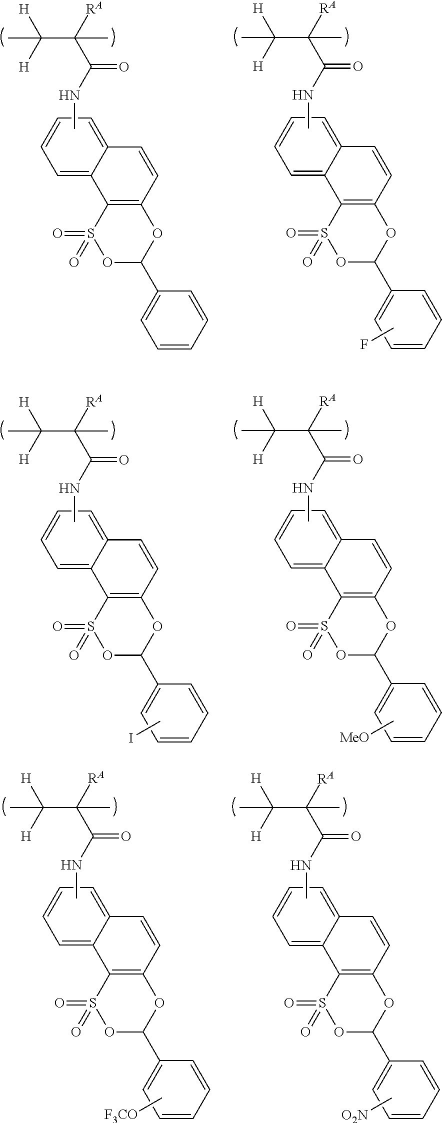

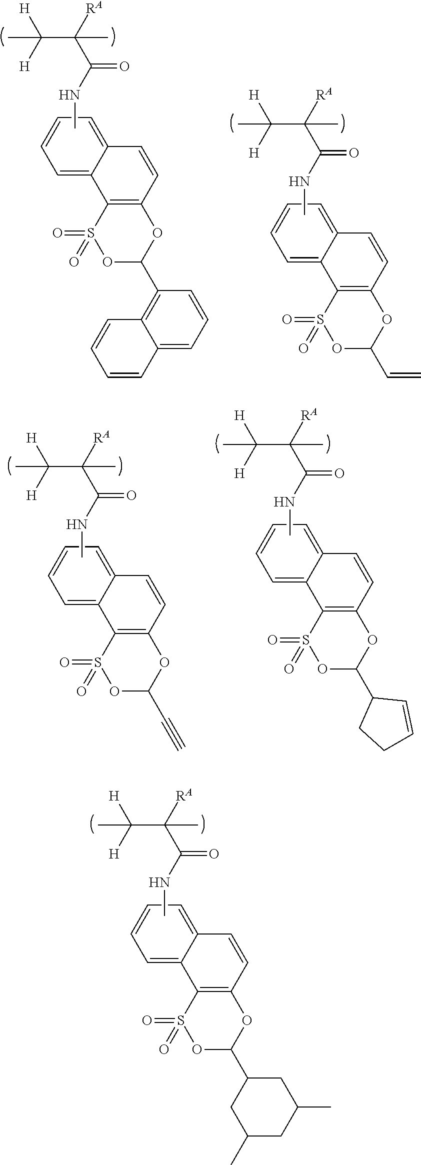

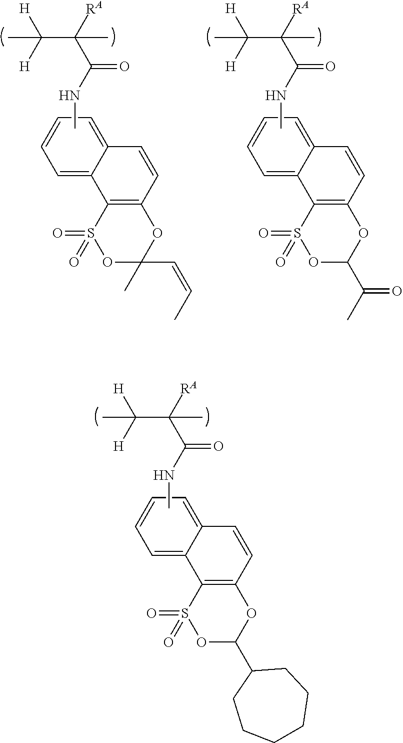

The base polymer preferably comprises repeat units having the formula (bi), which are also referred to as repeat units (bl), or repeat units having the formula (b2), which are also referred to as repeat units (b2).

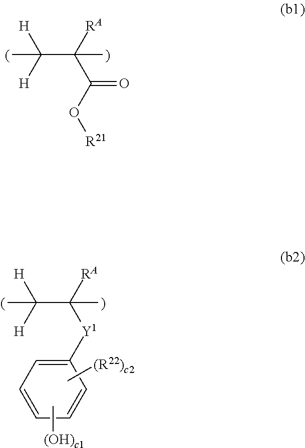

In formulae (b1) and (b2), R^{A} is each independently hydrogen, fluorine, methyl or trifluoromethyl. Y¹ is a single bond or *—C(═O)—O—. The asterisk (*) designates a point of attachment to the carbon atom in the backbone. R²¹ is hydrogen or a C₁-C₂₀ group containing at least one structure selected from among hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—). R²² is halogen, carboxy, nitro, cyano, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. A plurality of R²² may be identical or different when c2 is 2, 3, or 4. c1 is 1, 2, 3, or 4. c2 is 0, 1, 2, 3, or 4. Notably, 1≤c1+c2≤5.

Examples of the repeat units (b1) include those shown below, but not limited thereto. In the formulae, R^{A} is as defined above.

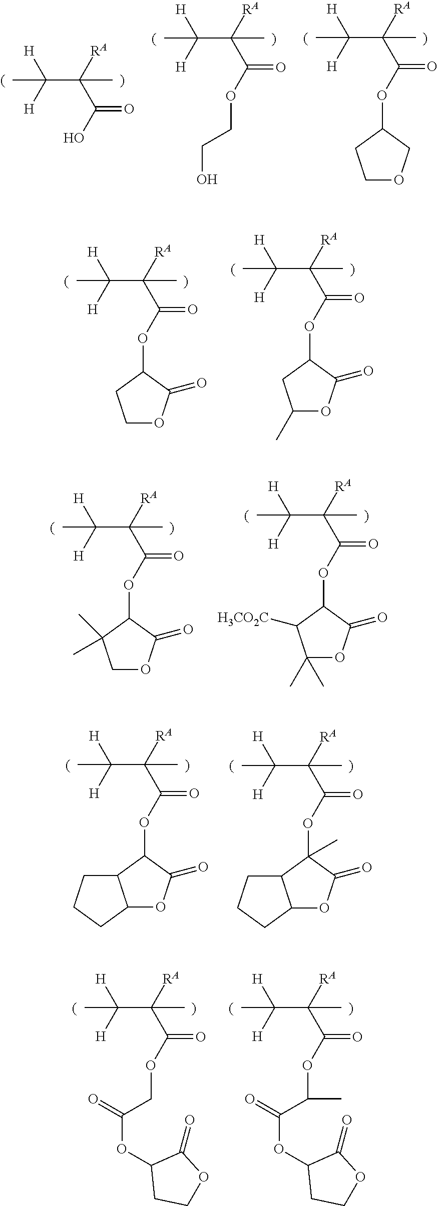

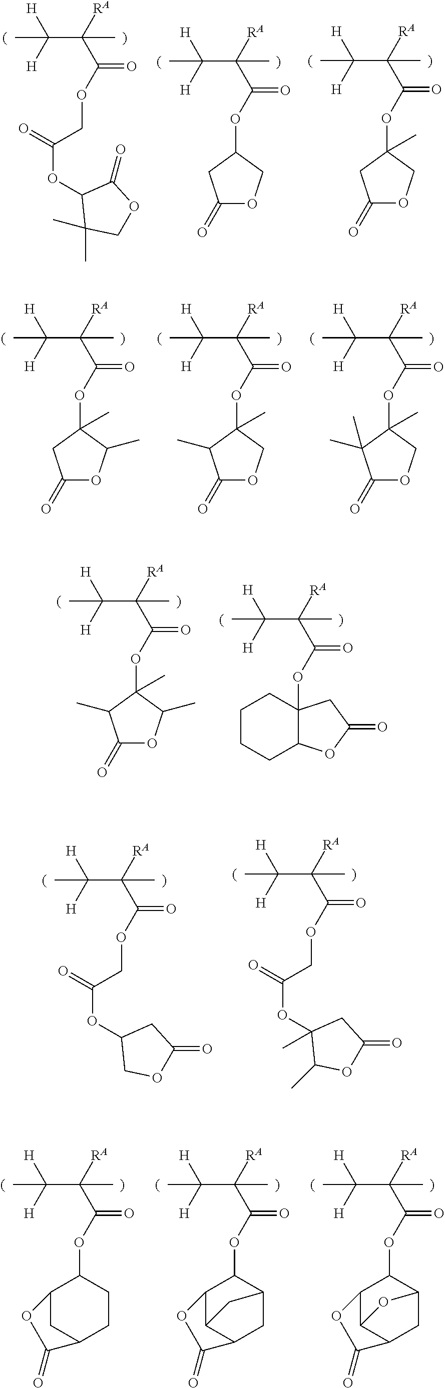

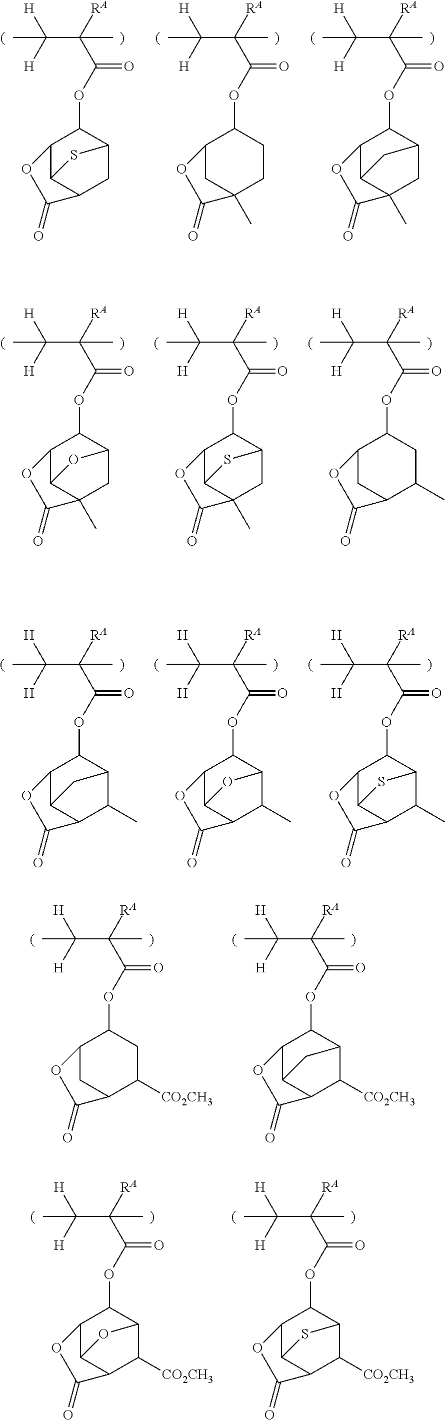

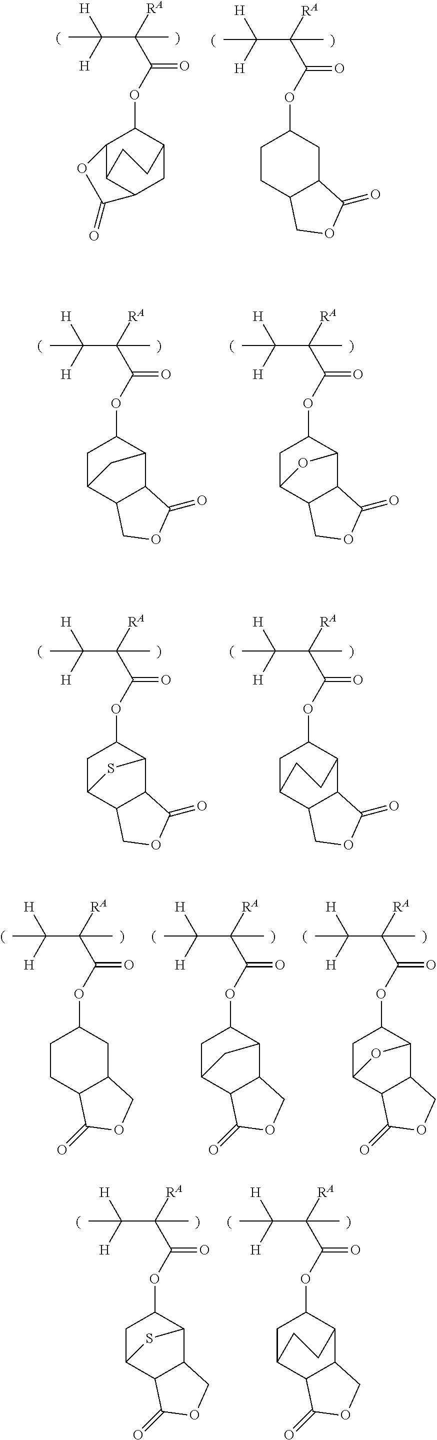

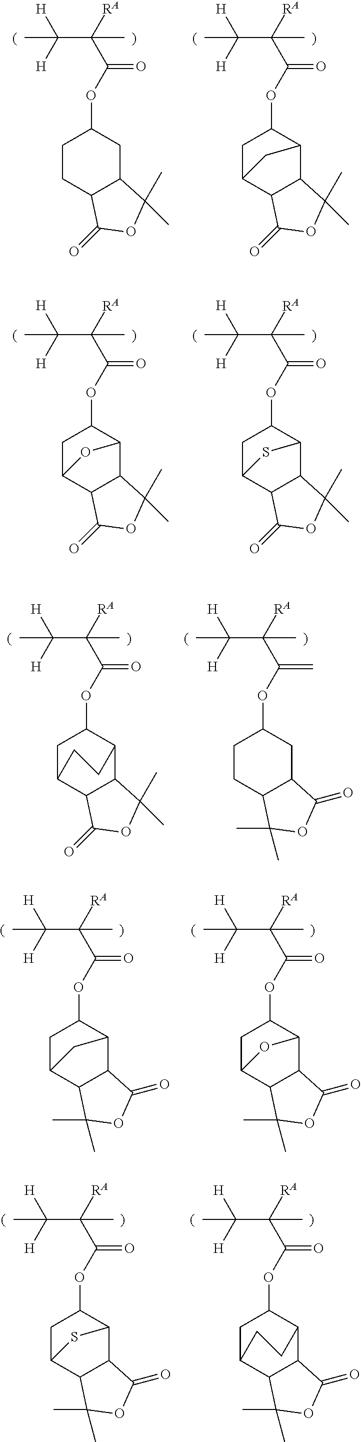

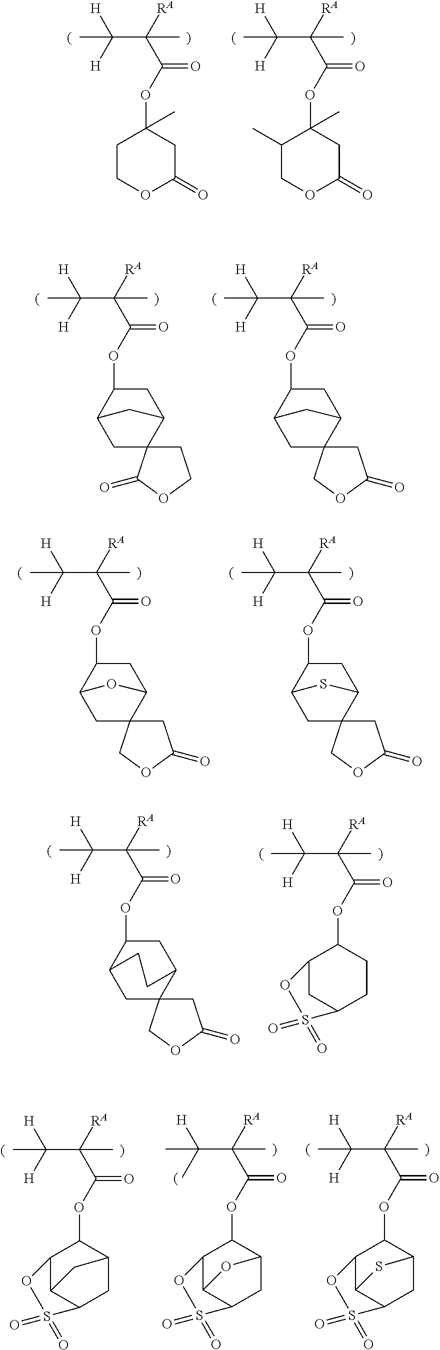

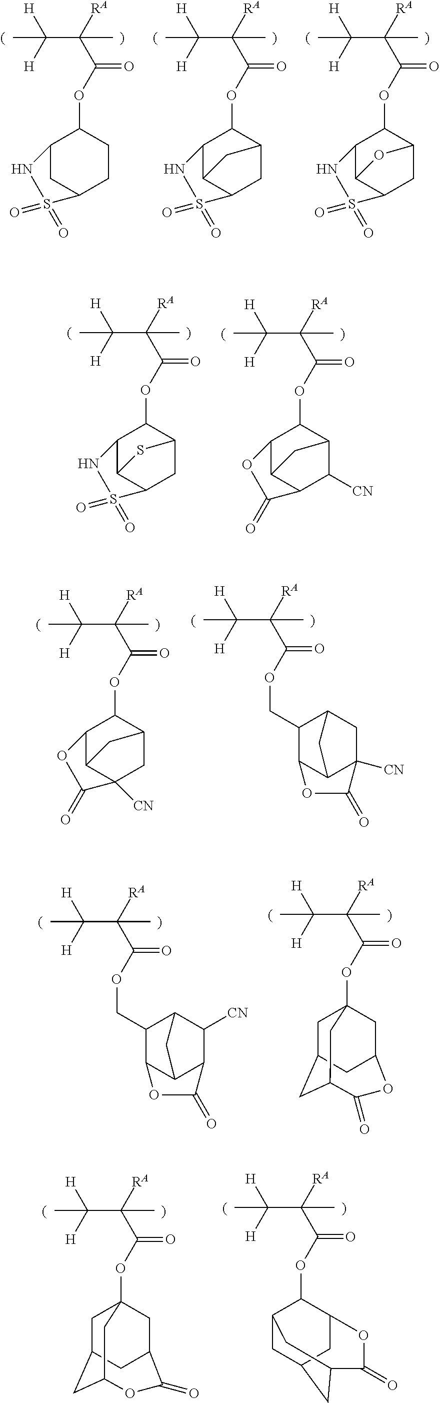

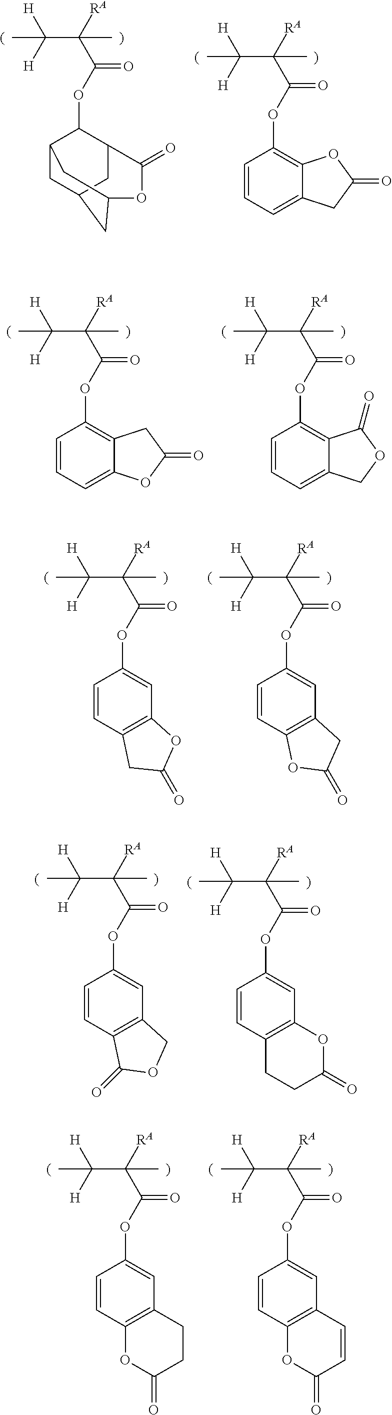

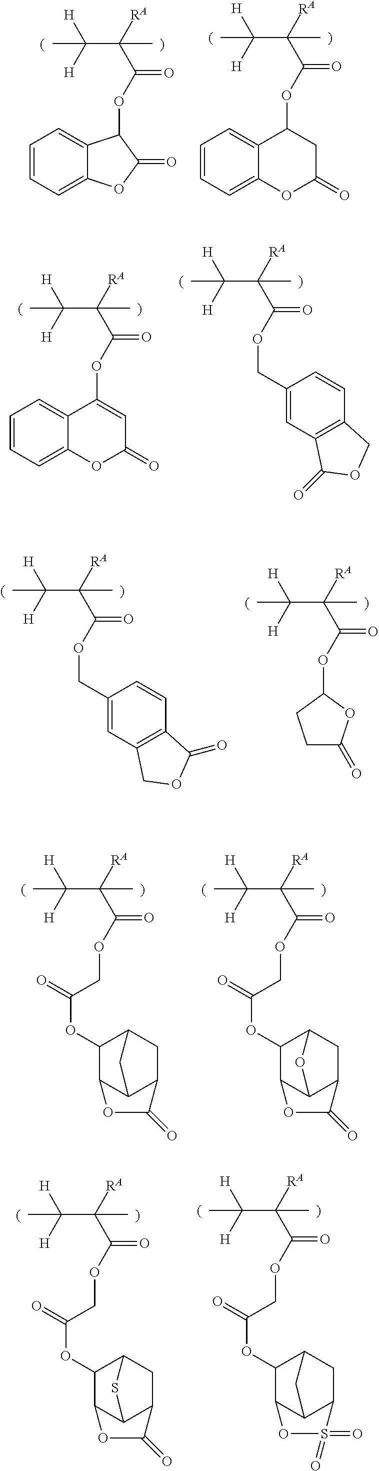

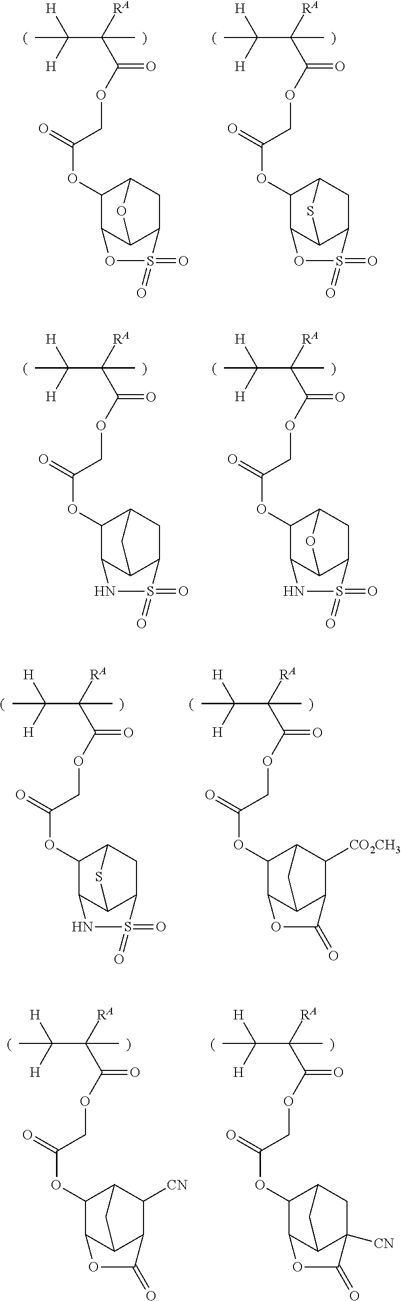

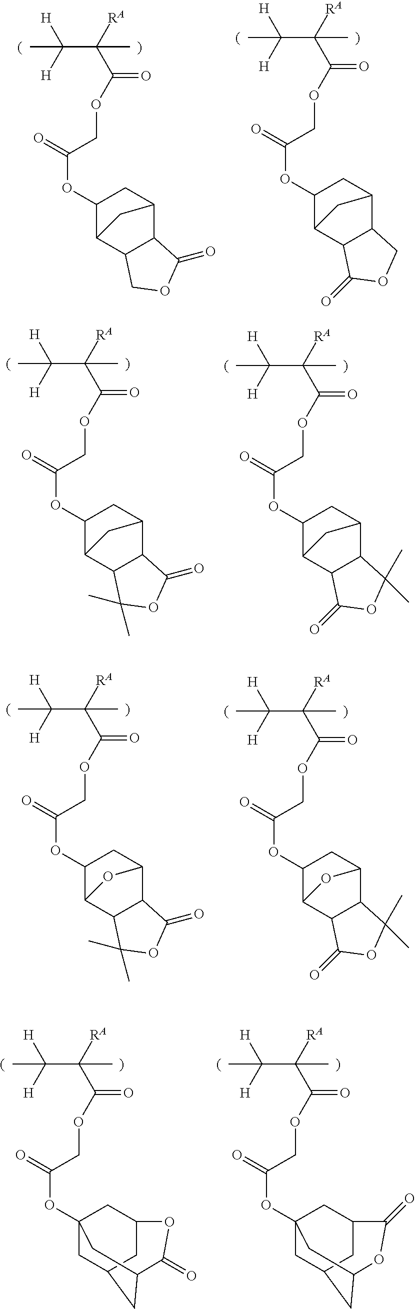

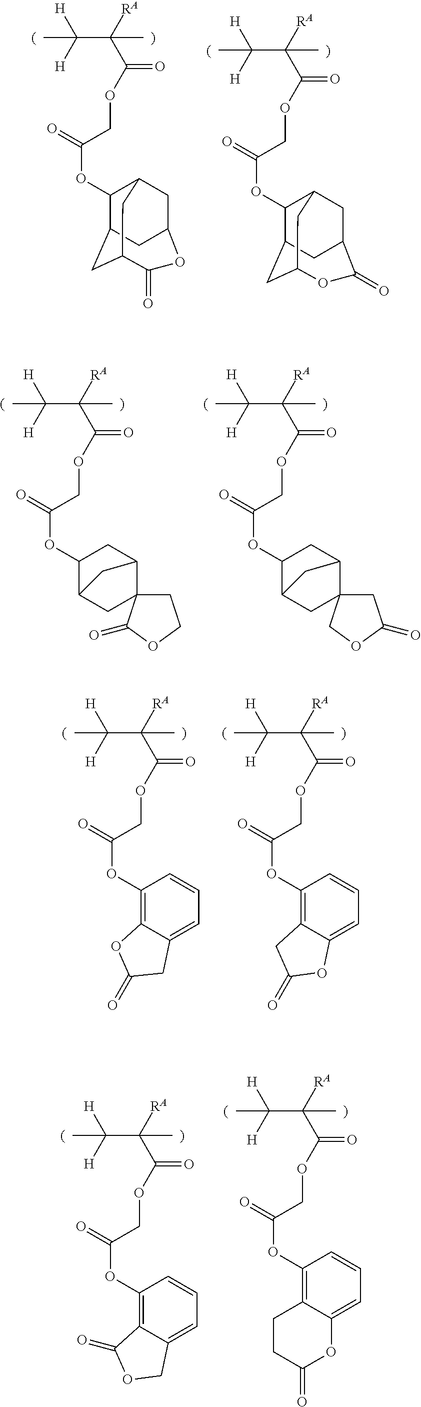

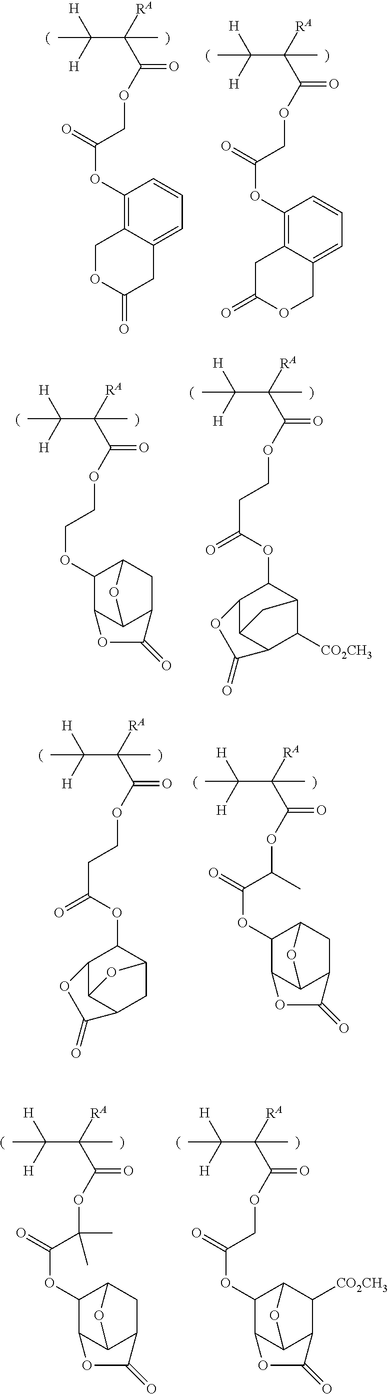

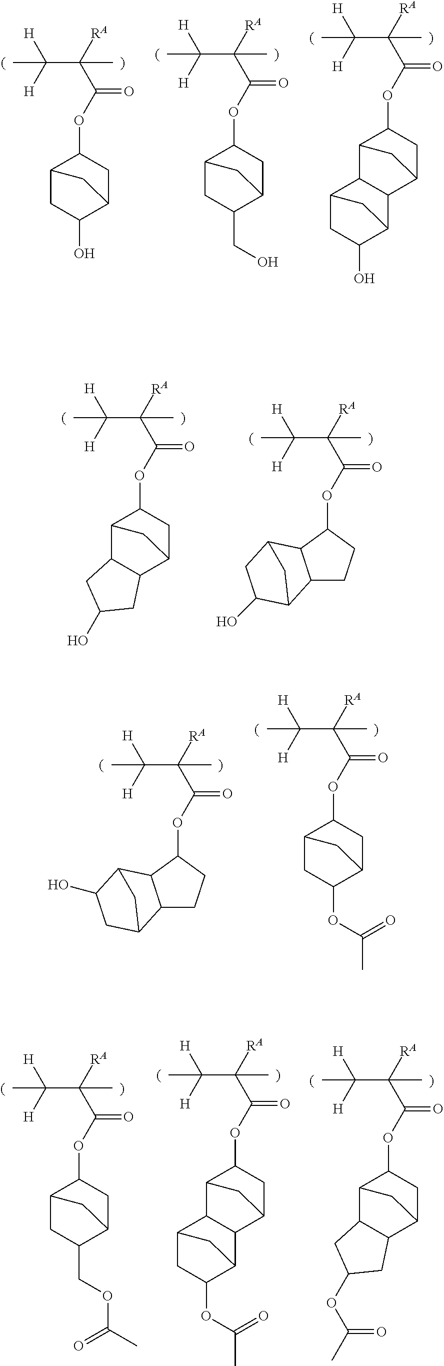

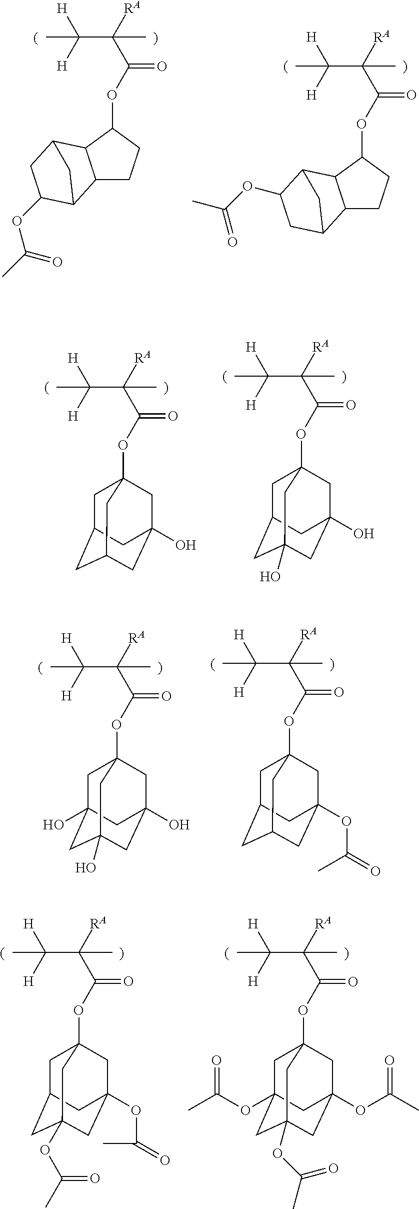

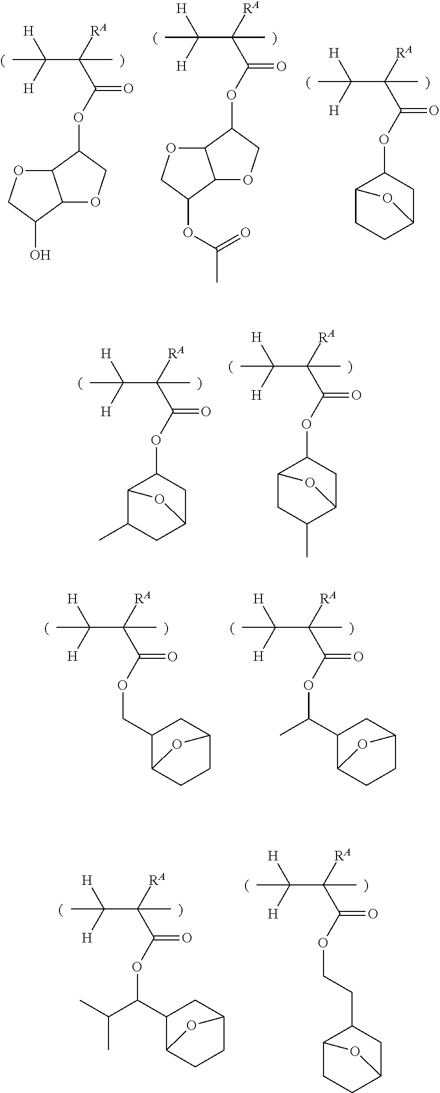

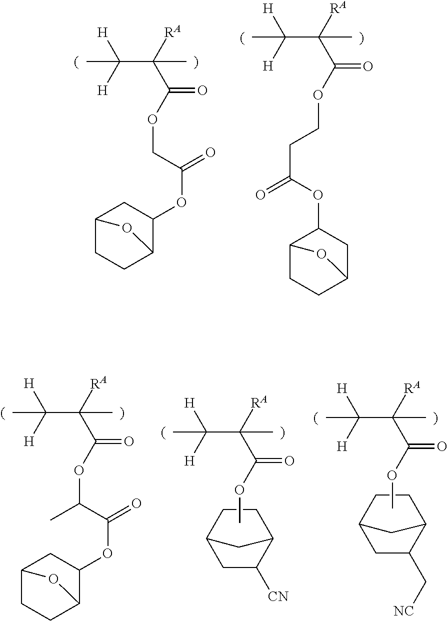

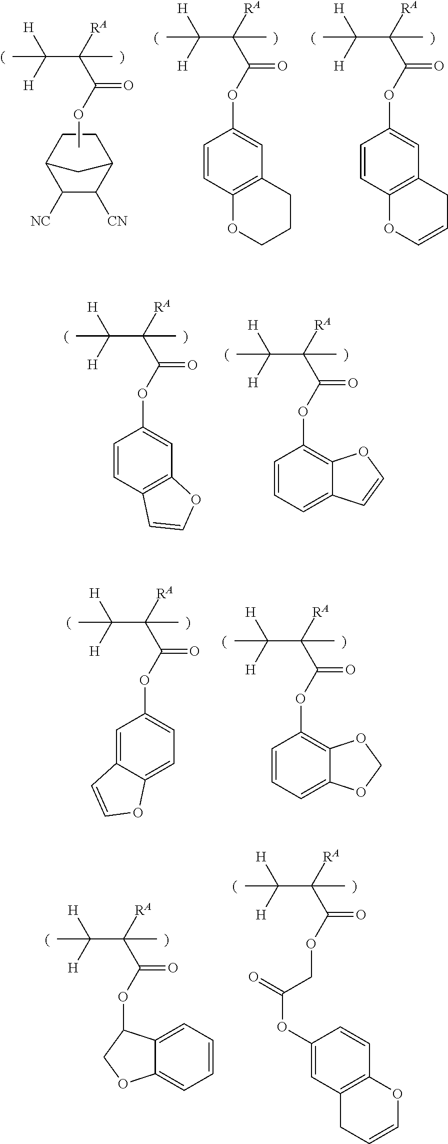

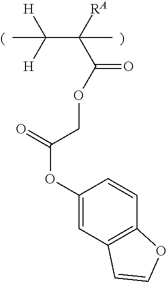

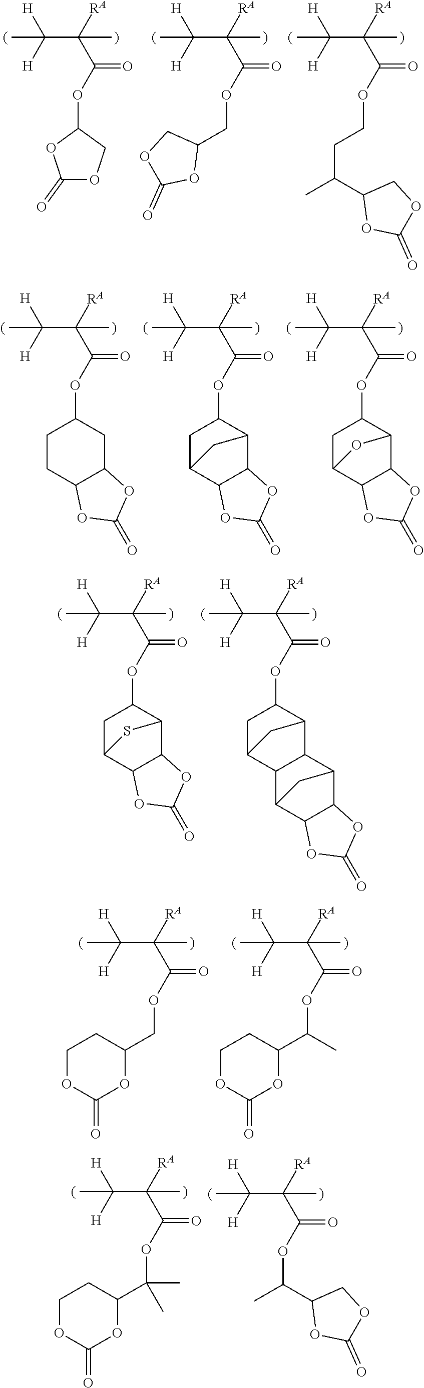

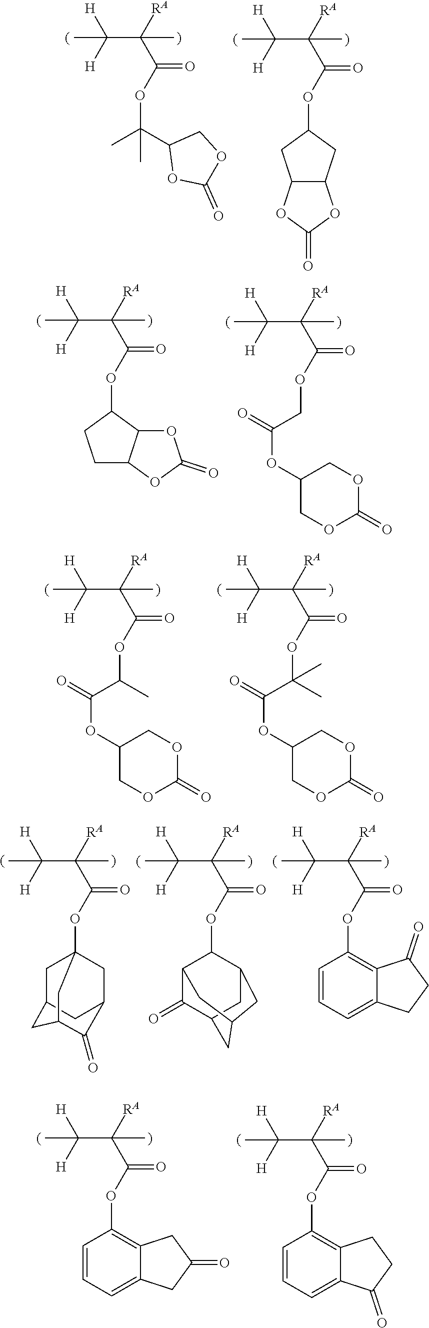

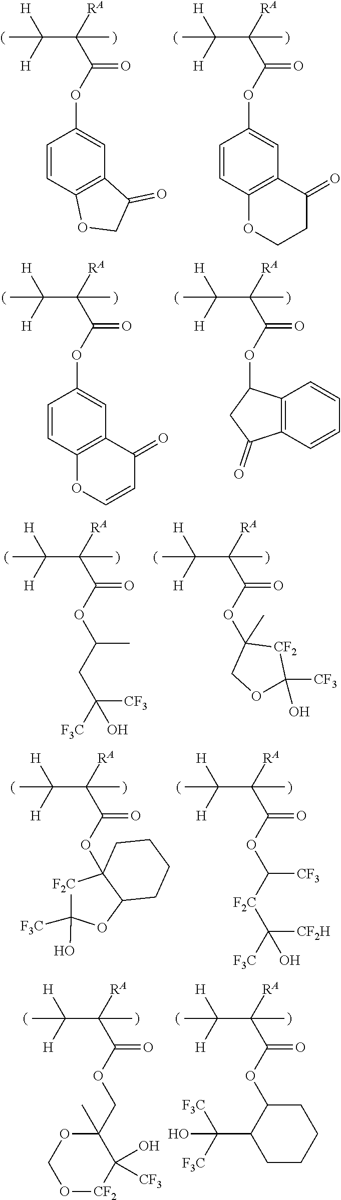

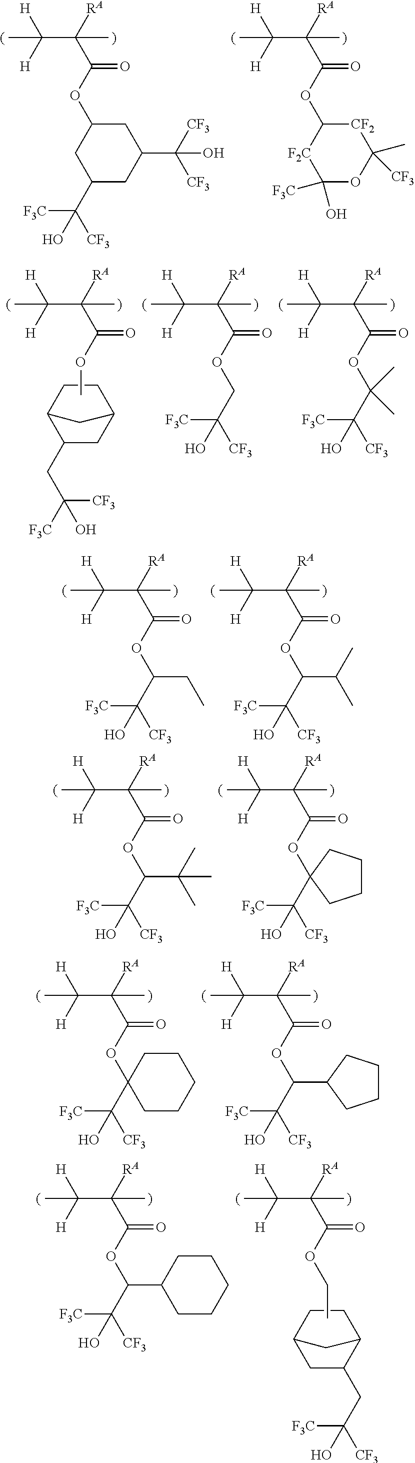

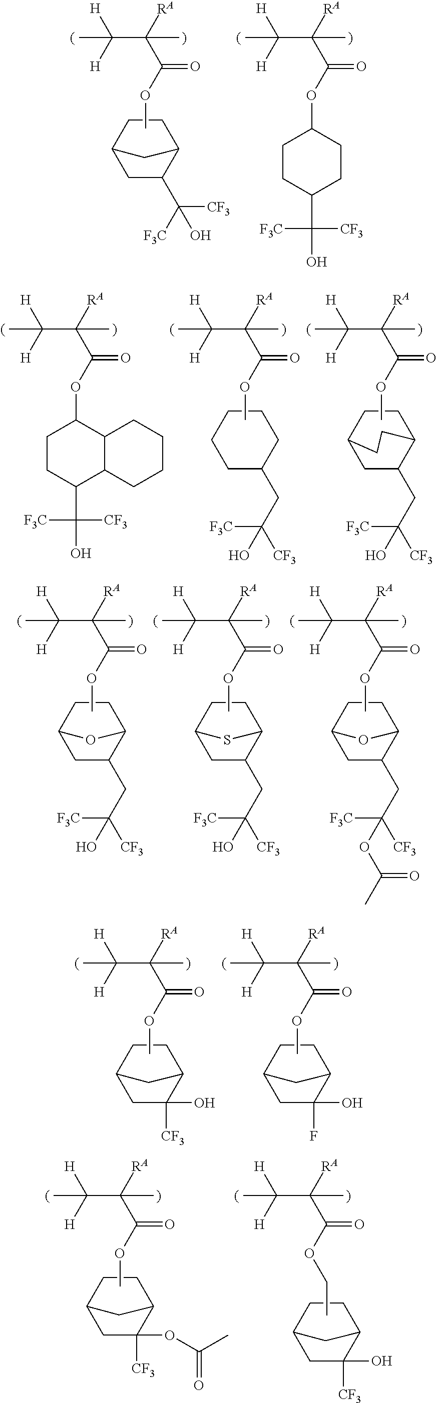

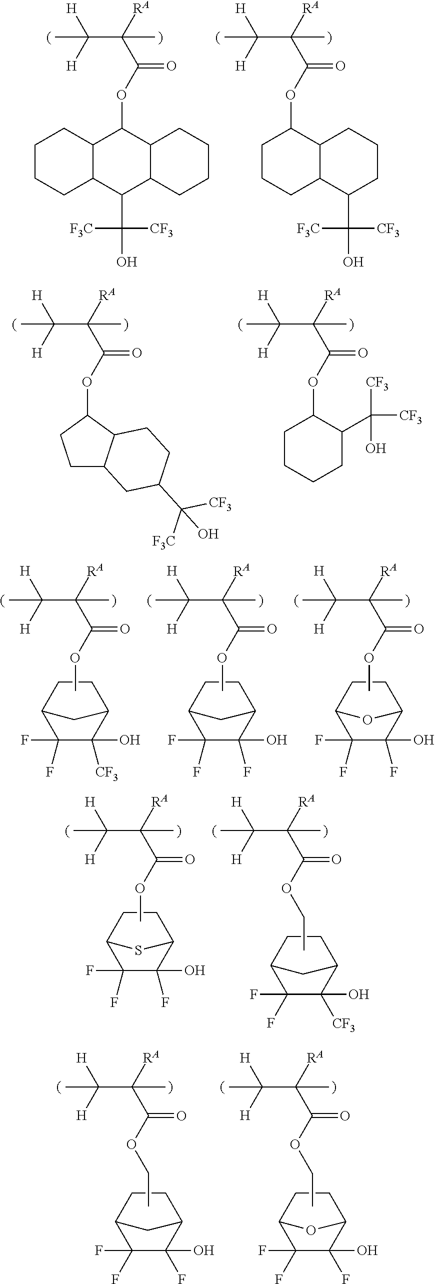

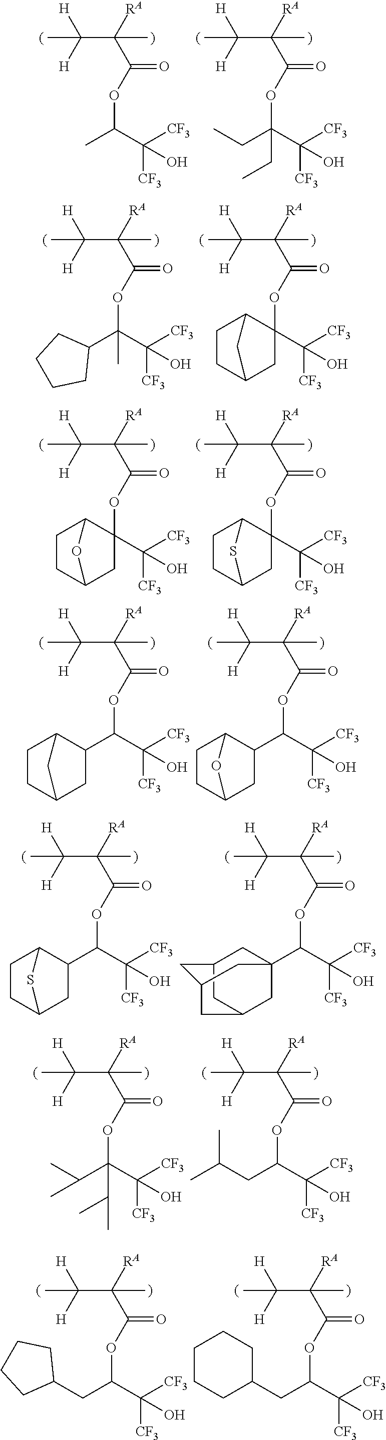

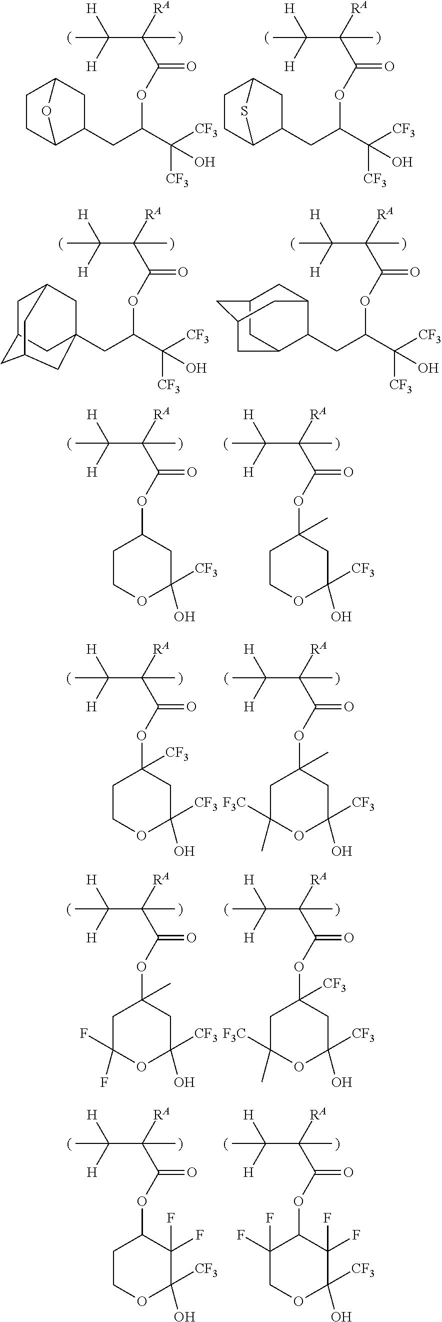

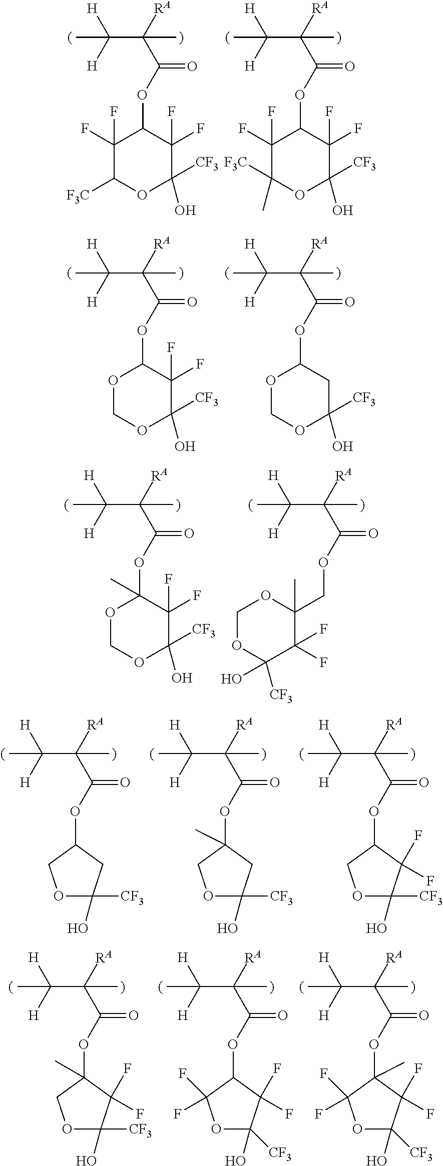

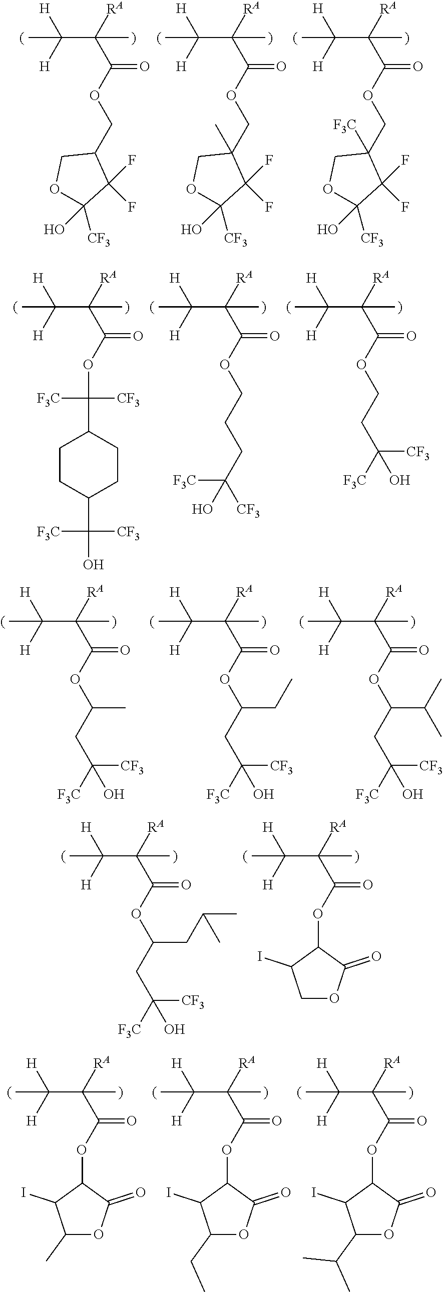

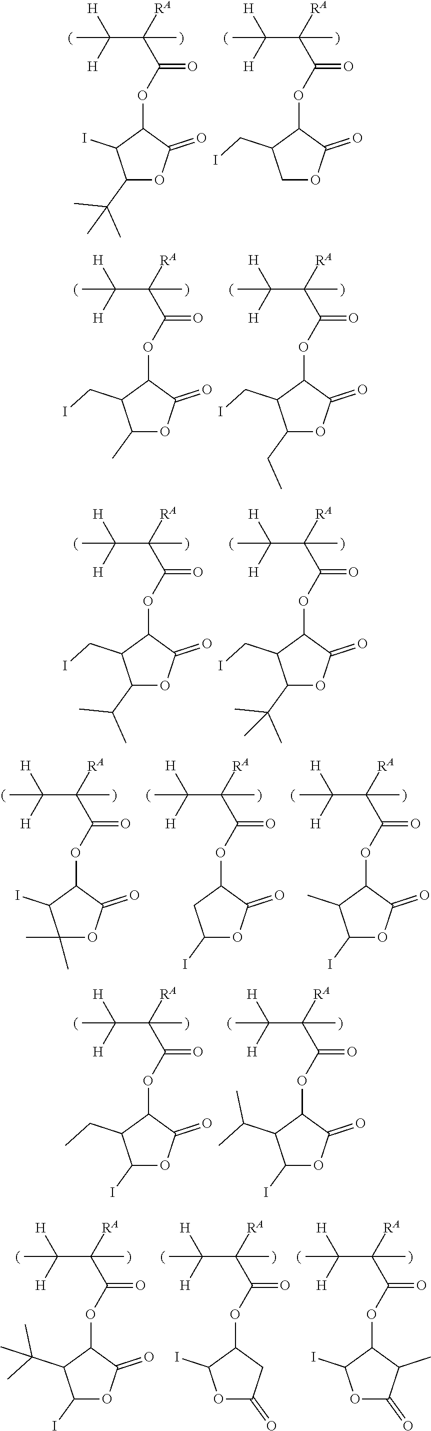

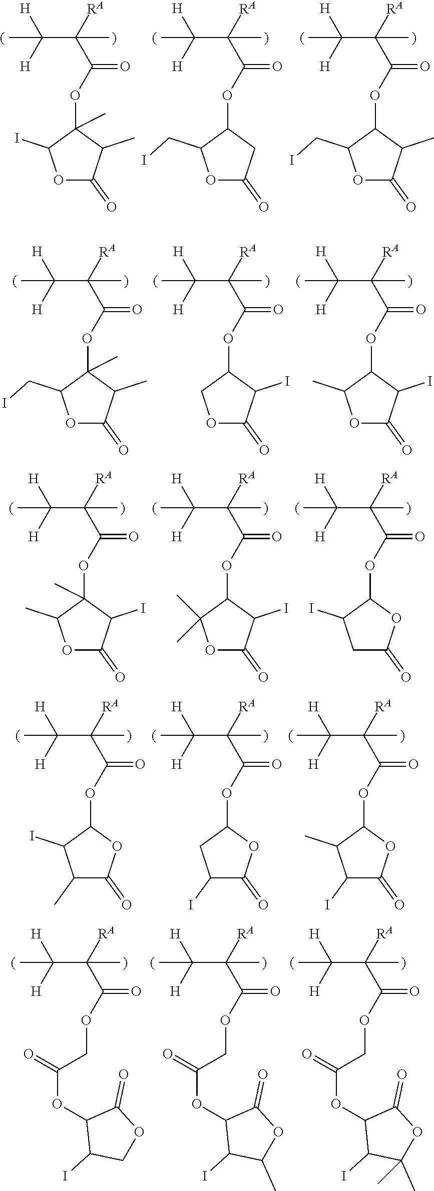

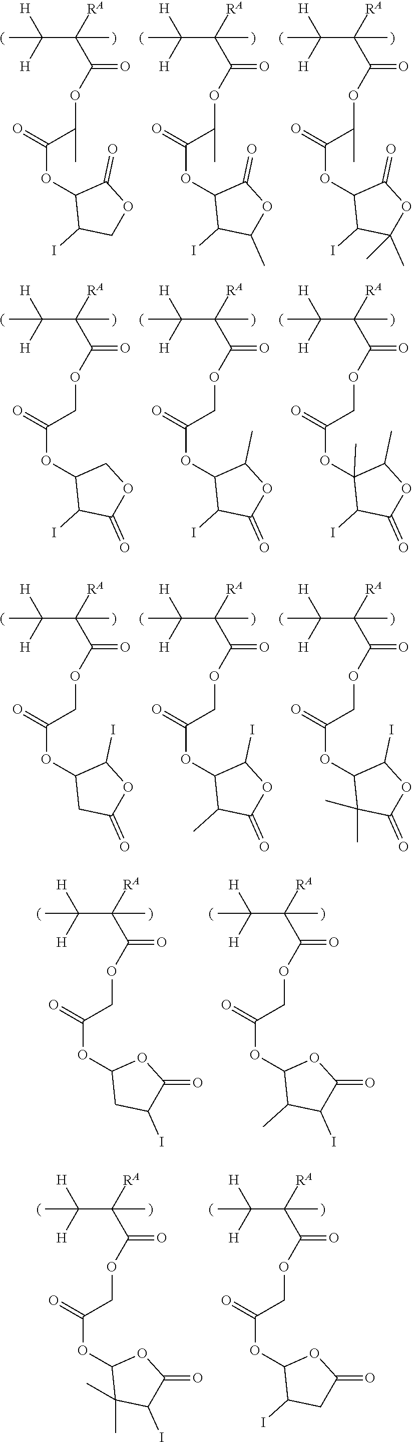

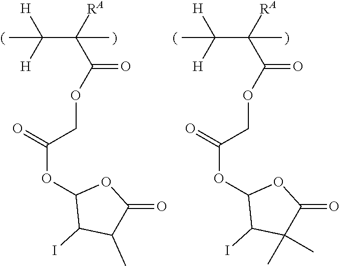

Examples of the repeat units (b2) include those shown below, but not limited thereto. In the formulae, R^{A} is as defined above.

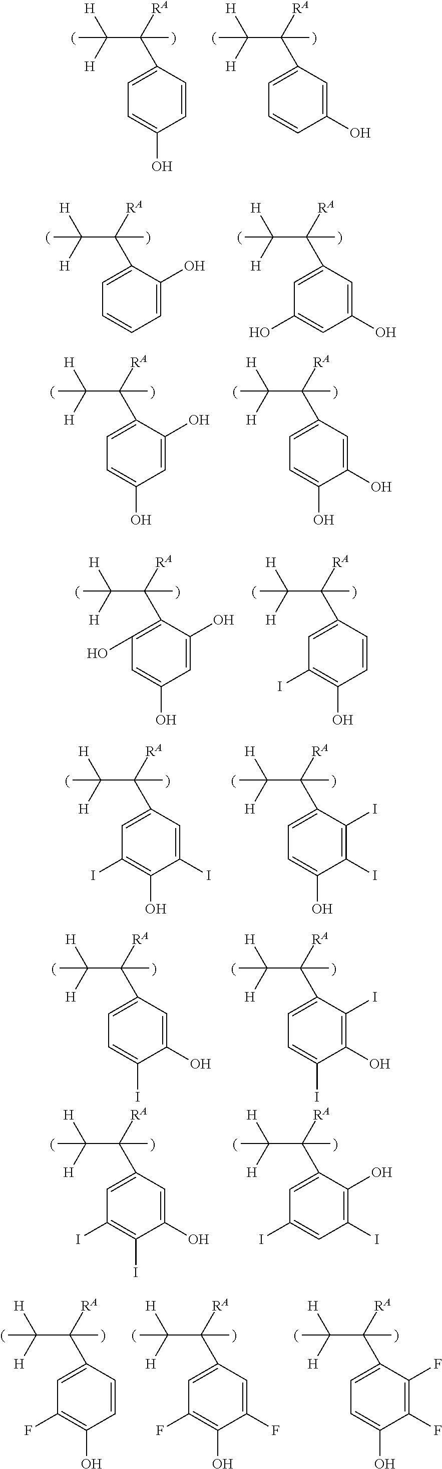

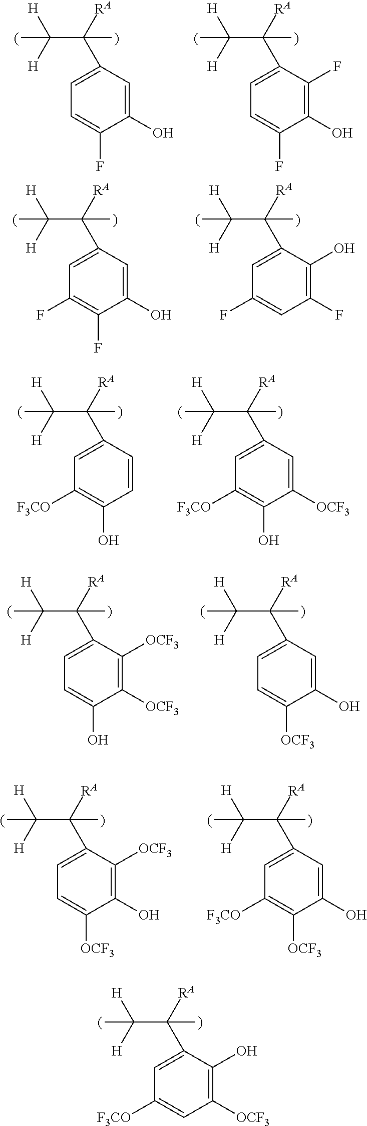

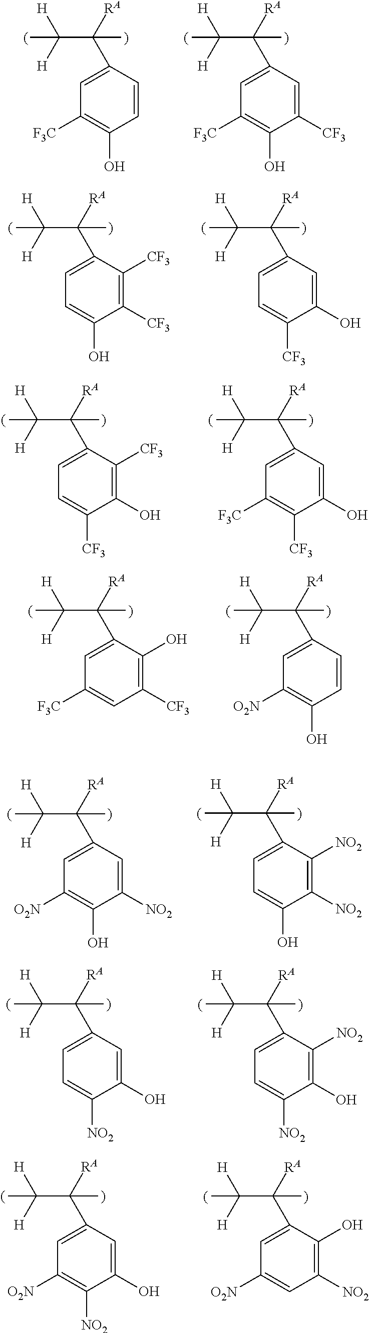

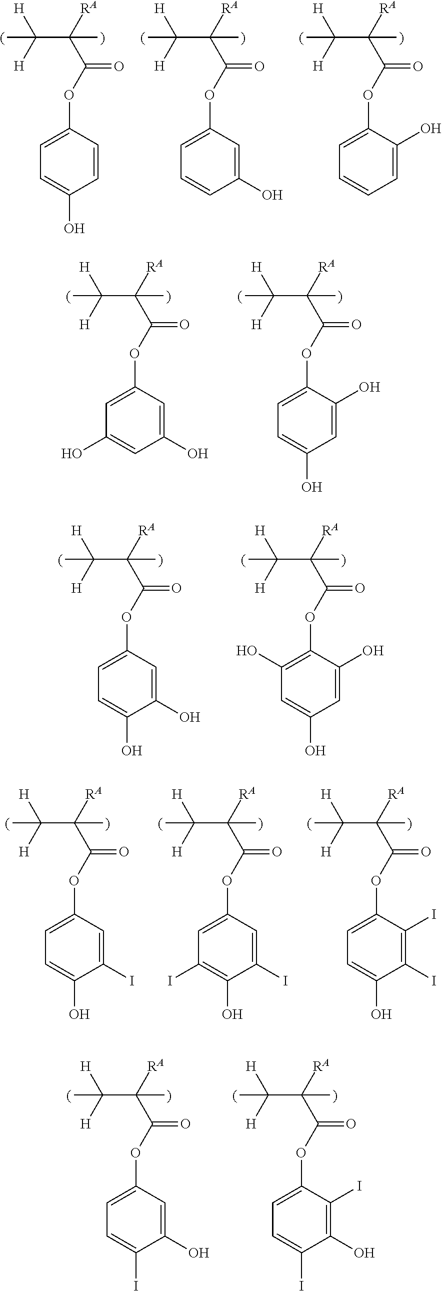

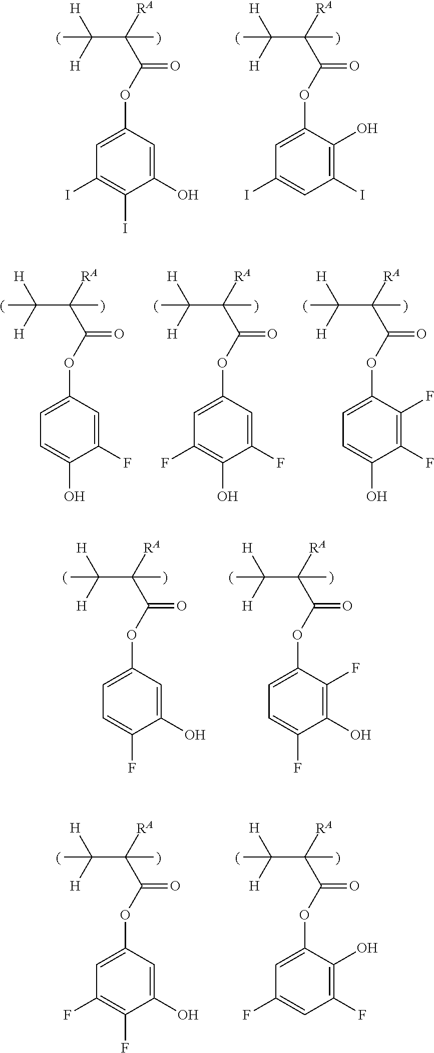

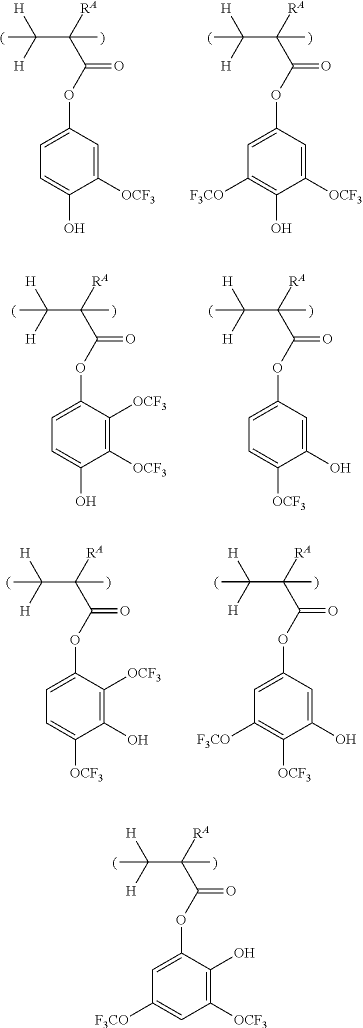

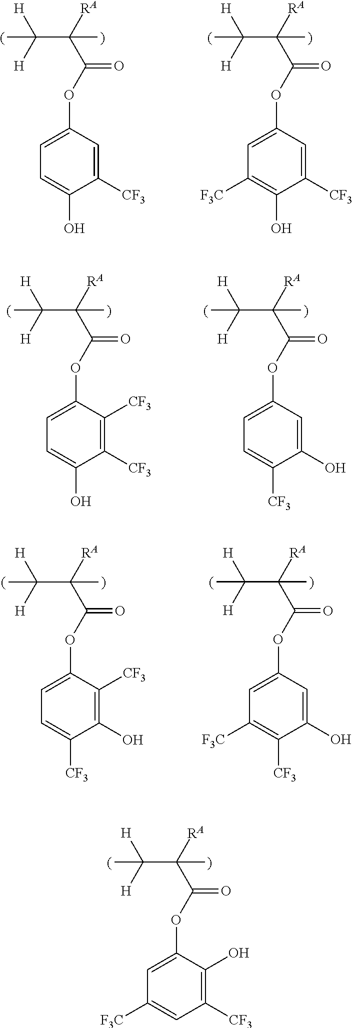

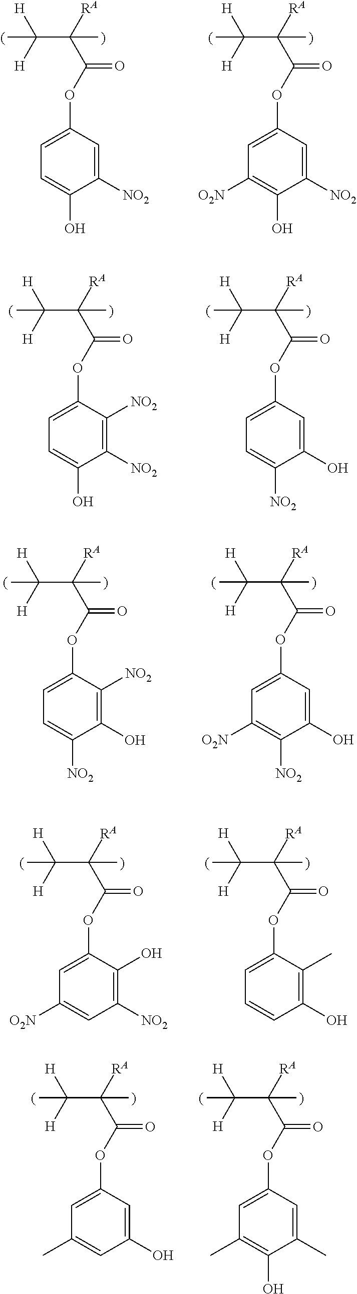

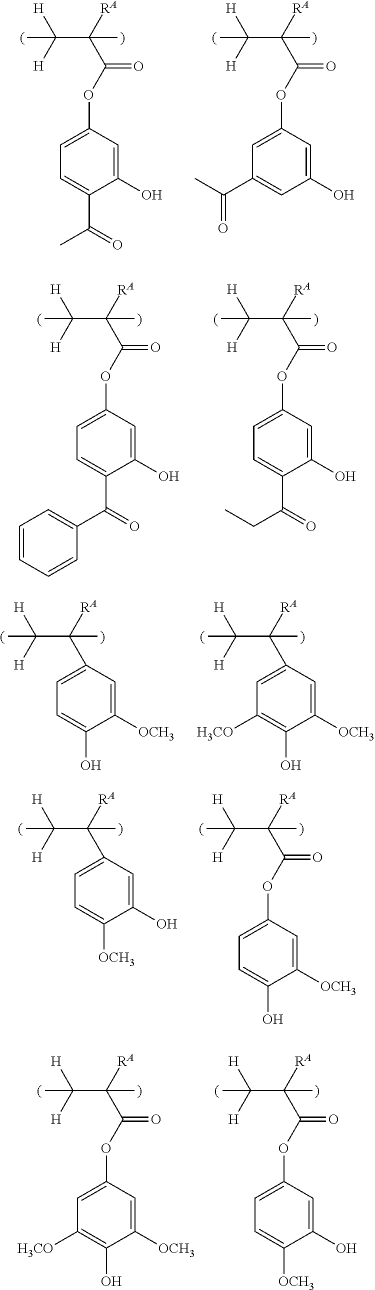

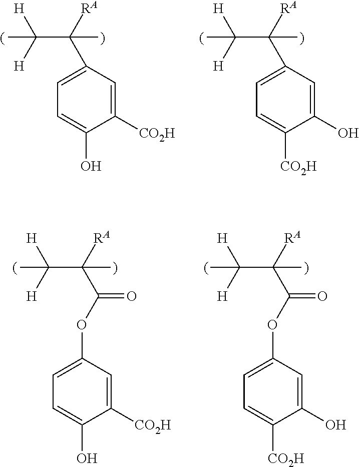

Of the repeat units (bl) or (b2), those units having a lactone ring as the polar group are particularly preferred in the ArF lithography and those units having a phenolic site are preferred in the KrF, EB and EUV lithography.

The base polymer may comprise at least one selected from repeat units having the formula (c1), which are also referred to as repeat units (c1), repeat units having the formula (c2), which are also referred to as repeat units (c2), repeat units having the formula (c3), which are also referred to as repeat units (c3), repeat units having the formula (c4), which are also referred to as repeat units (c4), and repeat units having the formula (c5), which are also referred to as repeat units (c5).

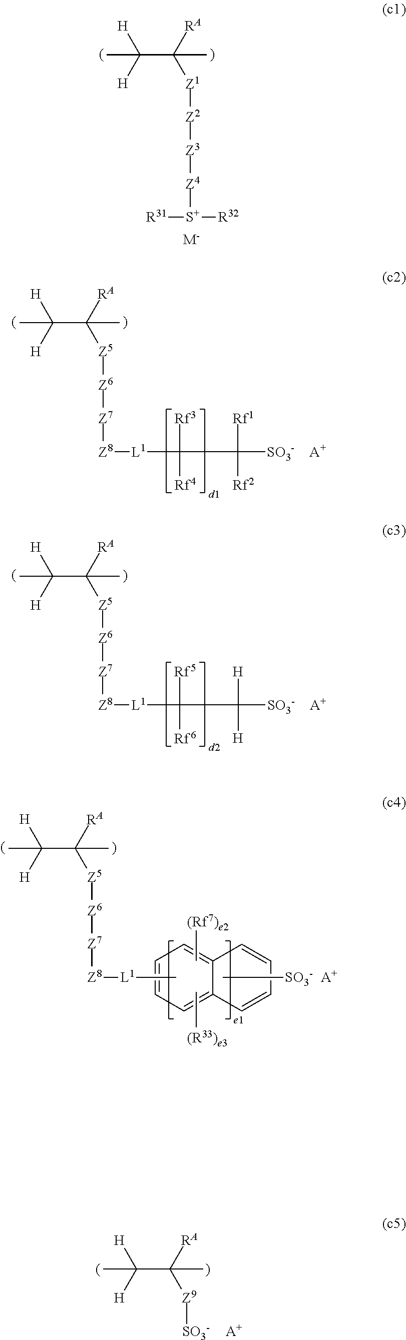

In the formulae (c1) to (c5), R^{A} is each independently hydrogen, fluorine, methyl or trifluoromethyl. Z¹ is a single bond or optionally substituted phenylene group. Z² is a single bond, **—C(═O)—O—Z²¹—, **—C(═O)—NH—Z²¹—, or **—O—Z²¹—. Z²¹ is a C₁-C₆ aliphatic hydrocarbylene group, phenylene group or a divalent group obtained by combining the foregoing, which may contain halogen, a carbonyl moiety, ester bond, ether bond or hydroxy moiety. Z³ is a single bond, ether bond, ester bond, amide bond, sulfonate ester bond, sulfonamide bond, carbonate bond or carbamate bond. Z⁴ is a single bond, or a C1-C₆ aliphatic hydrocarbylene group, phenylene group or a divalent group obtained by combining the foregoing, which may contain halogen, carbonyl moiety, ester bond, ether bond or hydroxy moiety. Z⁵ is each independently a single bond, optionally substituted phenylene group, naphthylene group, or *—C(═O)—O—Z⁵¹. Z⁵¹ is a C₁-C₁₀ aliphatic hydrocarbylene group which may contain halogen, hydroxy moiety, ether bond, ester bond or lactone ring, or phenylene or naphthylene group. Z⁶ is a single bond, ether bond, ester bond, amide bond, sulfonate ester bond, sulfonamide bond, carbonate bond or carbamate bond. Z⁷ is each independently a single bond, ***—Z⁷¹—C(═O)—O—, ***—C(═O)—NH—Z⁷¹—, or ***—O—Z⁷¹—. Z⁷¹ is a C₁-C₂₀ hydrocarbylene group which may contain a heteroatom. Z⁸ is each independently a single bond, ****—Z⁸¹—C(═O)—O—, ****—C(═O)—NH—Z⁸¹—, or ****—O—Z⁸¹—. Z⁸¹ is a C₁-C₂₀ hydrocarbylene group which may contain a heteroatom. Z⁹ is a single bond, methylene, ethylene, phenylene, fluorinated phenylene, trifluoromethyl-substituted phenylene, *—C(═O)—O—Z⁹¹—, *—C(═O)—N(H)—Z⁹¹—, or *—O—Z⁹¹—. Z⁹¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety. the asterisk (*) designates a point of attachment to the carbon atom in the backbone, The asterisks (**) designate a point of attachment to Z¹. The asterisks (***) designate a point of attachment to Z⁶. The asterisks (****) designate a point of attachment to Z⁷.

The aliphatic hydrocarbylene group represented by Z²¹, Z⁵¹ and Z⁹¹ may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl, and cyclohexanediyl; and combinations thereof.

The hydrocarbylene group which may contain a heteroatom, represented by Z⁷¹ and Z⁸¹, may be saturated or unsaturated and straight, branched or cyclic. Examples thereof are shown below, but not limited thereto.

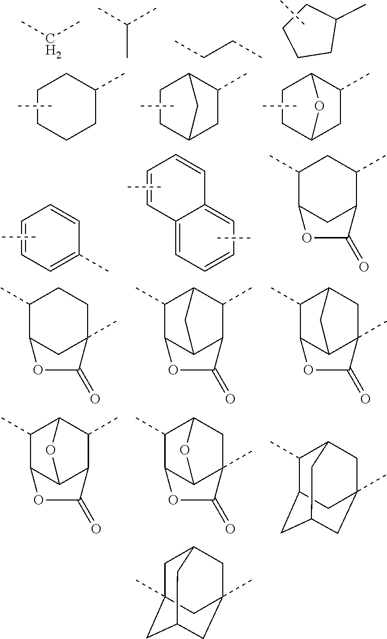

wherein the broken line represents a valence bond.

In the formula (c1), R³¹ and R³² are each independently a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, and tert-butyl; C₃-C₂₀ cyclic saturated hydrocarbyl groups such as cyclopropyl, cyclopentyl, cyclohexyl, cylopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C6-C₂₀ aryl groups such as phenyl, naphthyl, and thienyl; C₇-C₂₀ aralkyl groups such as benzyl, 1-phenylethyl, and 2-phenylethyl; and combinations thereof. Of these, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

R³¹ and R³² may bond together to form a ring with the sulfur atom to which they are attached. Examples of the ring include rings having the formulae:

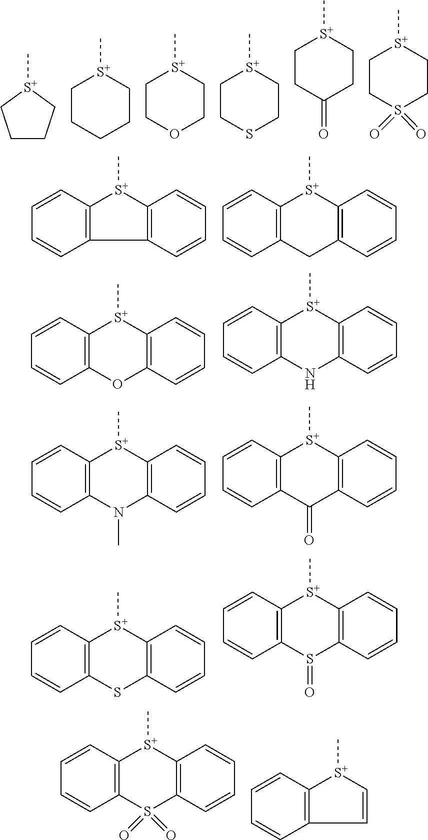

wherein the broken line is a point of attachment to Z⁴.

Examples of the cation in repeat unit (c1) include those shown below, but not limited thereto. In the formulae, R^{A} is as defined above.

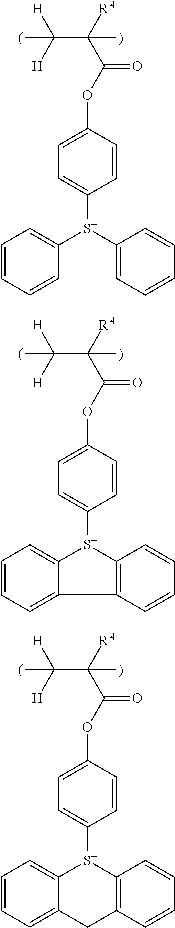

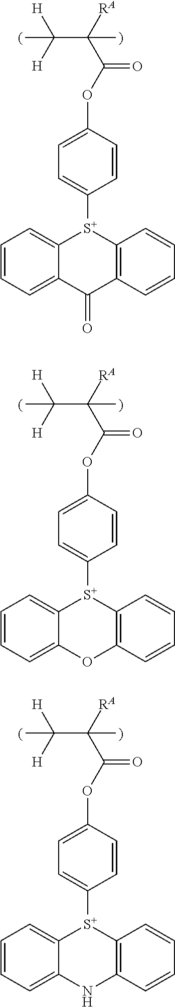

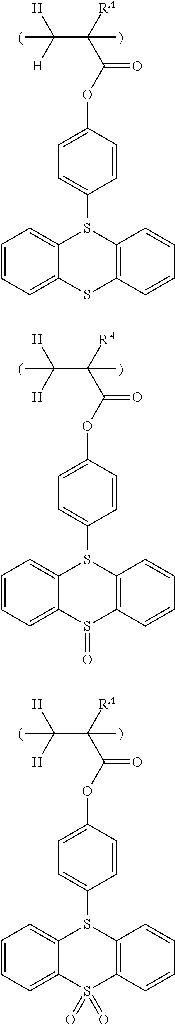

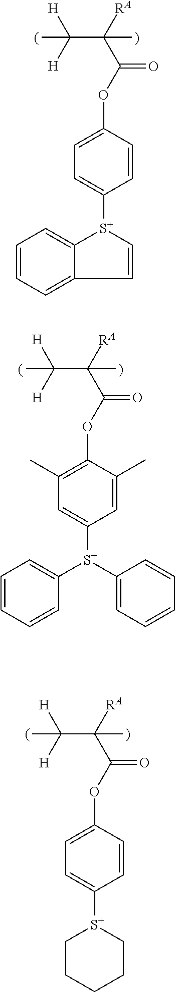

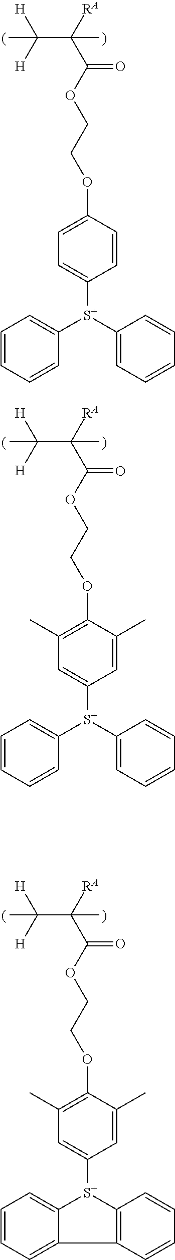

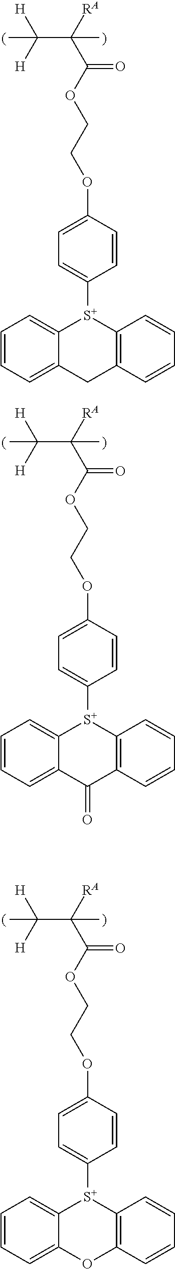

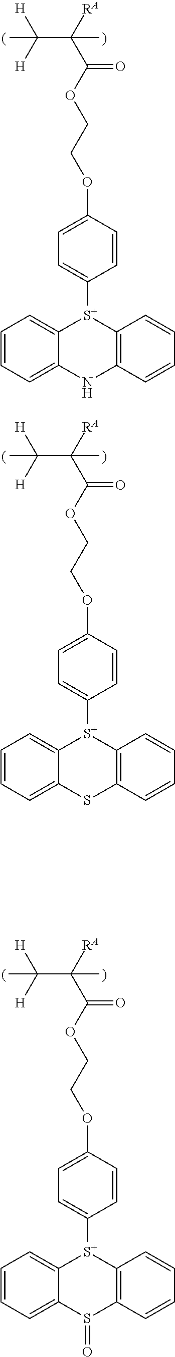

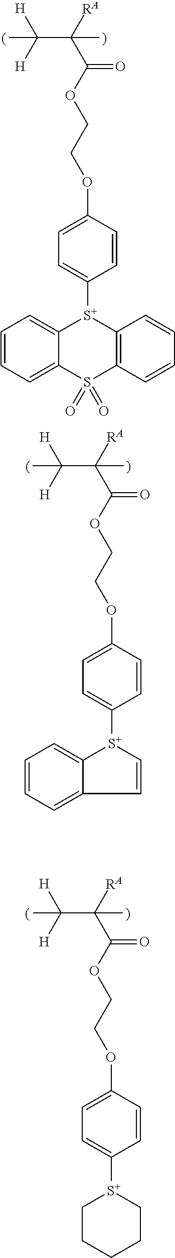

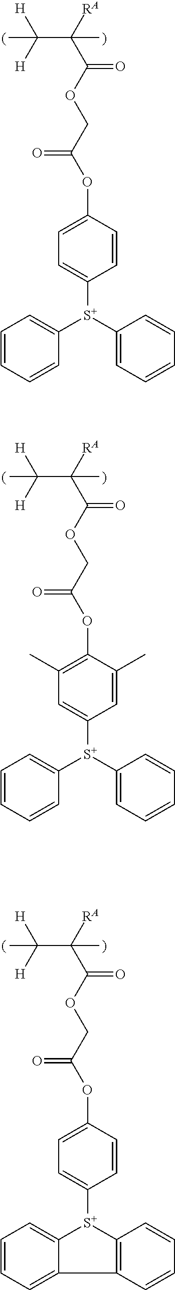

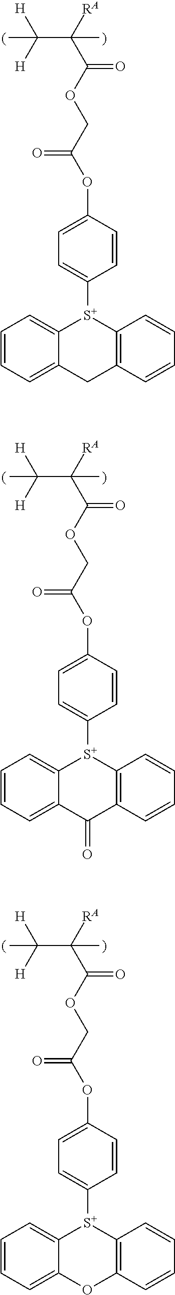

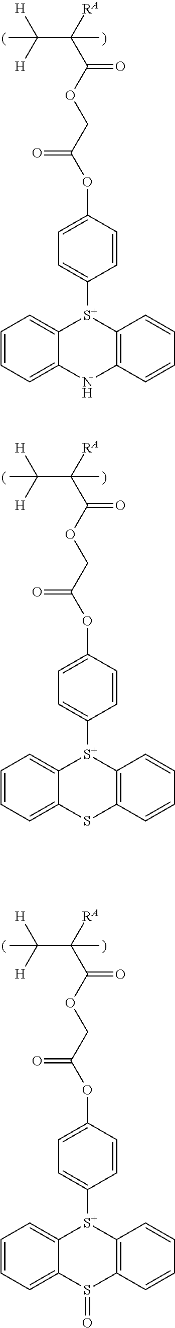

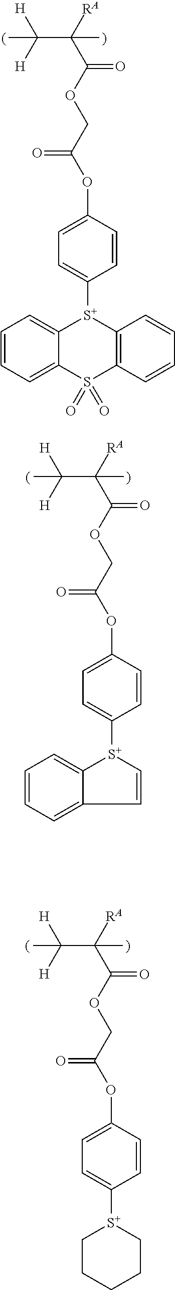

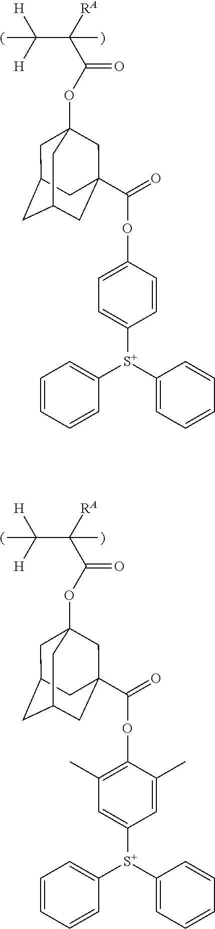

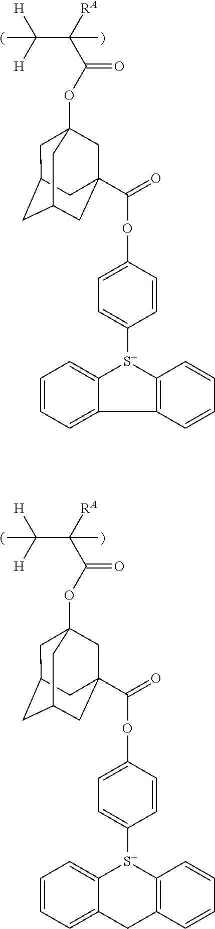

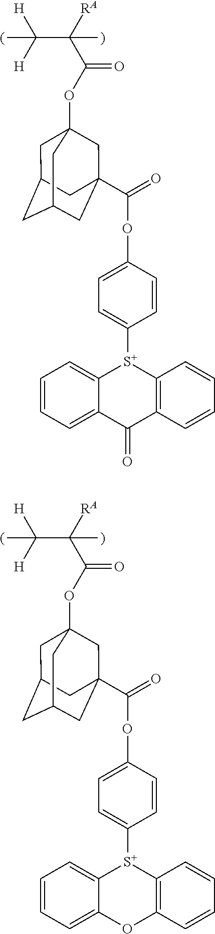

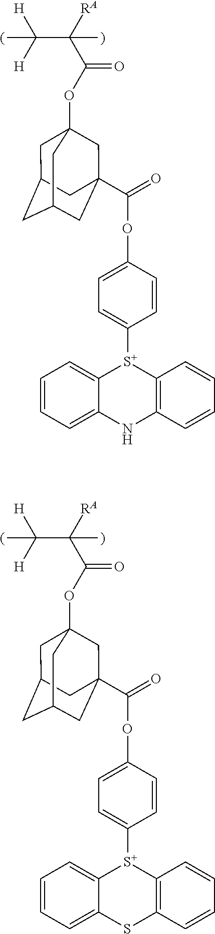

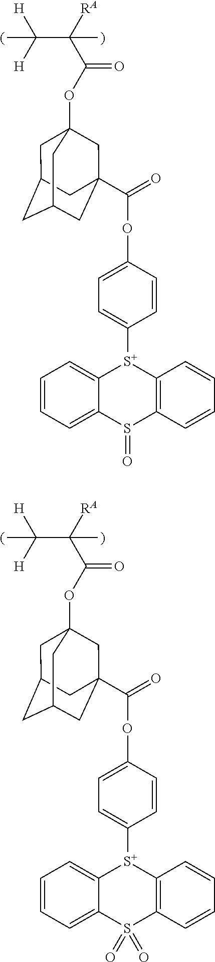

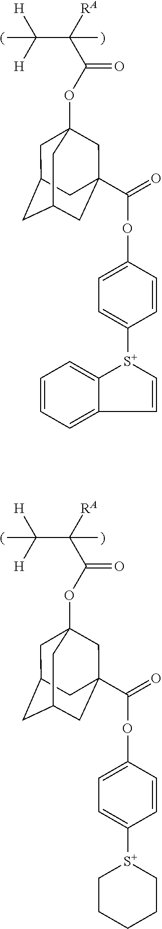

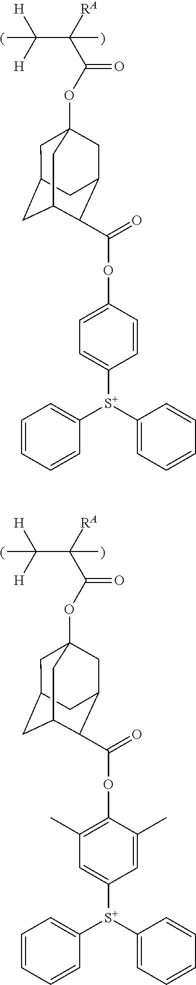

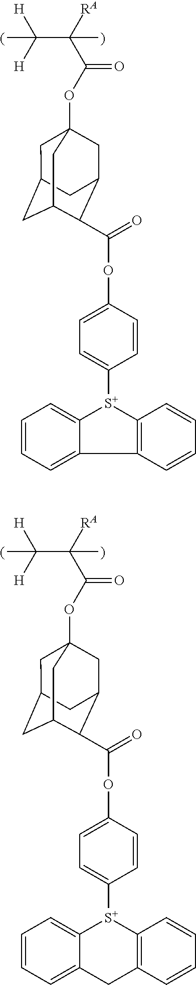

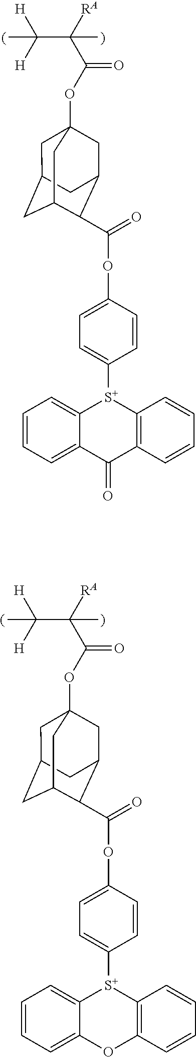

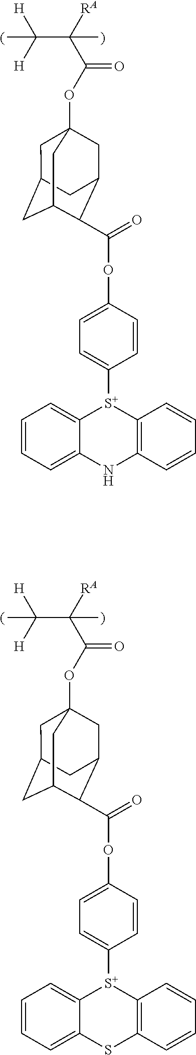

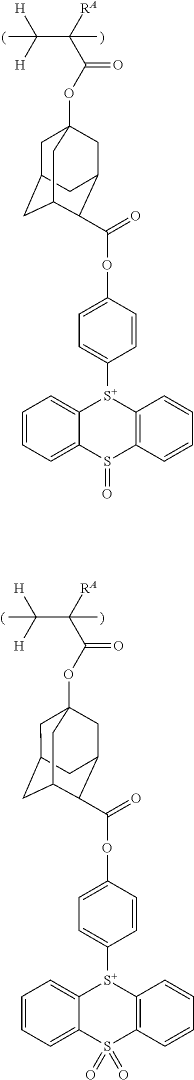

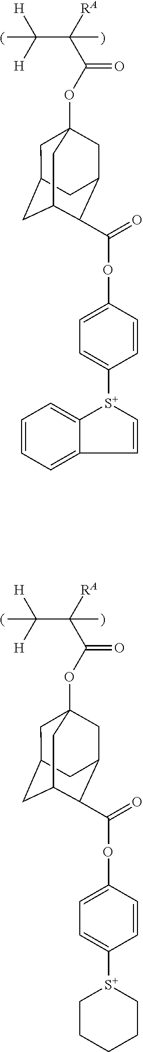

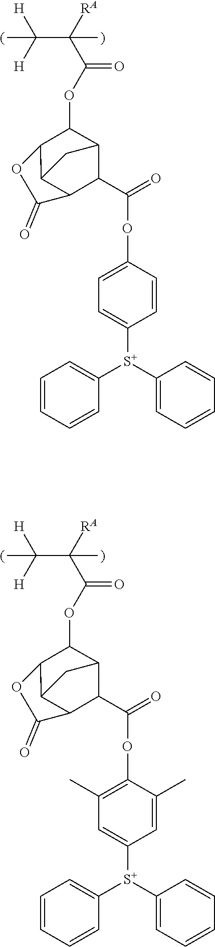

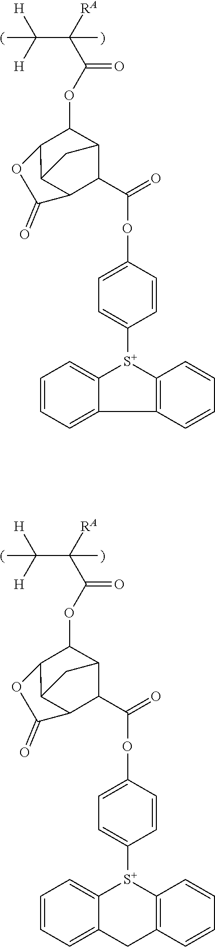

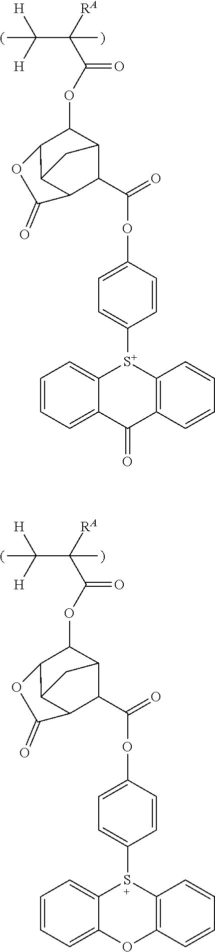

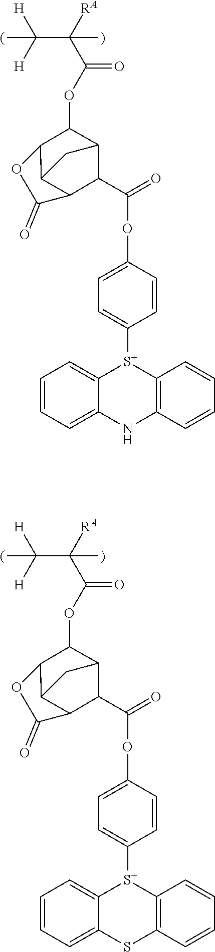

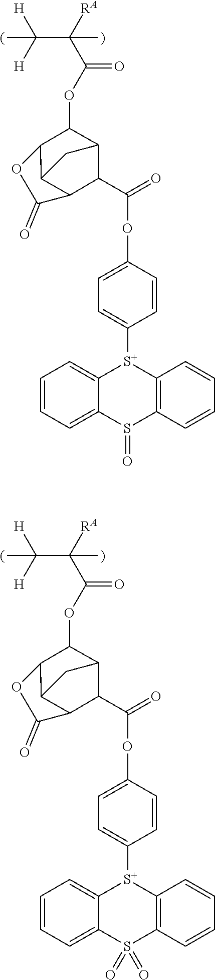

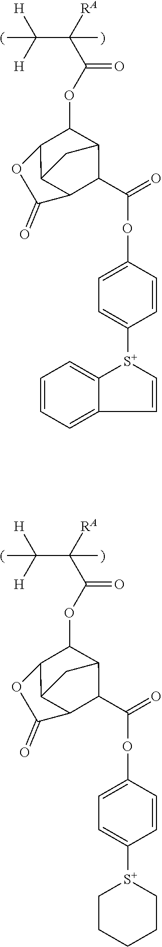

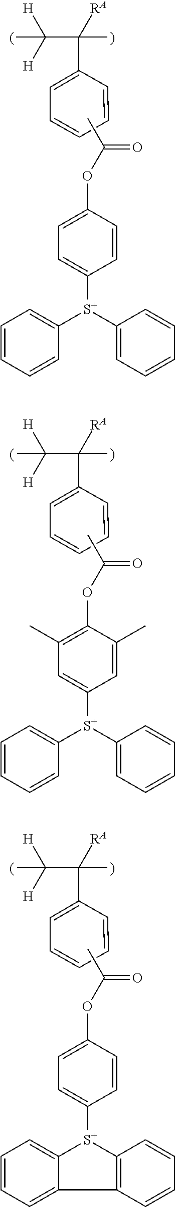

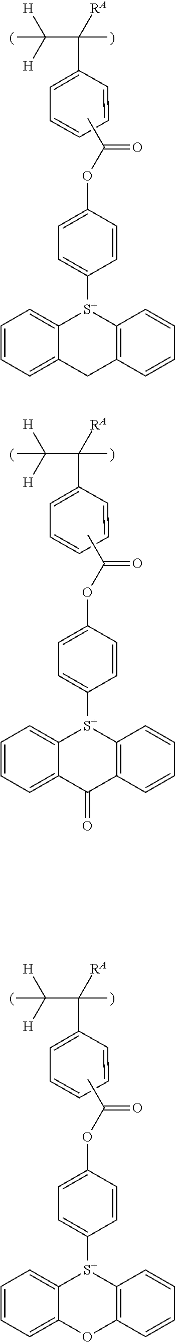

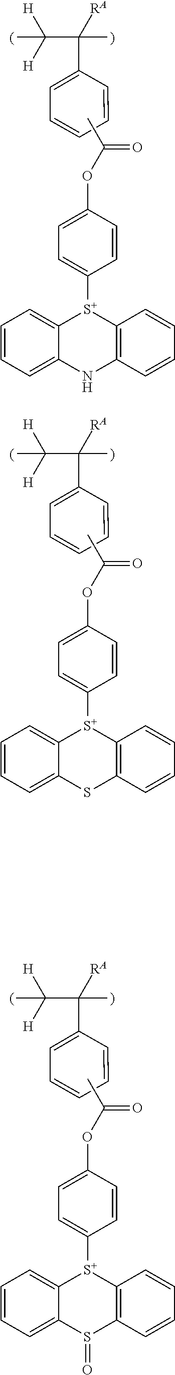

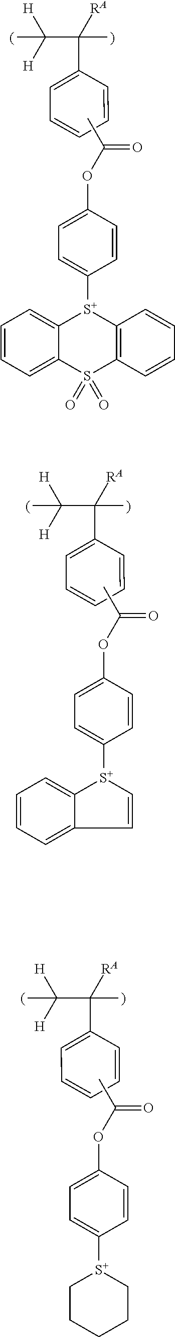

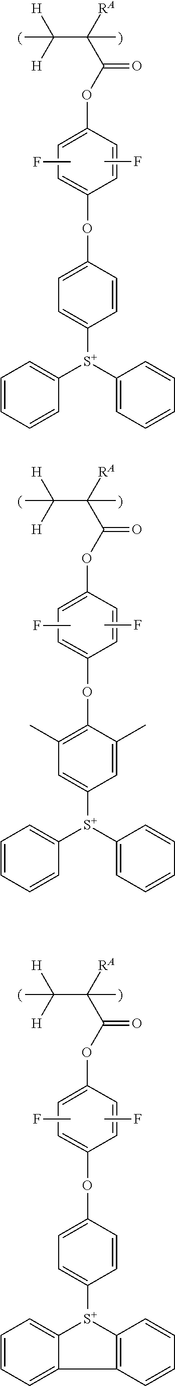

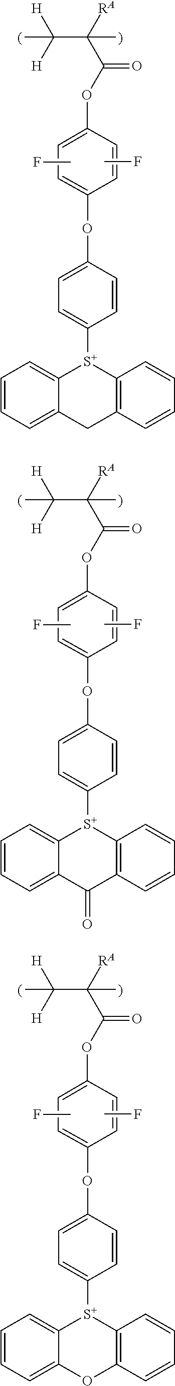

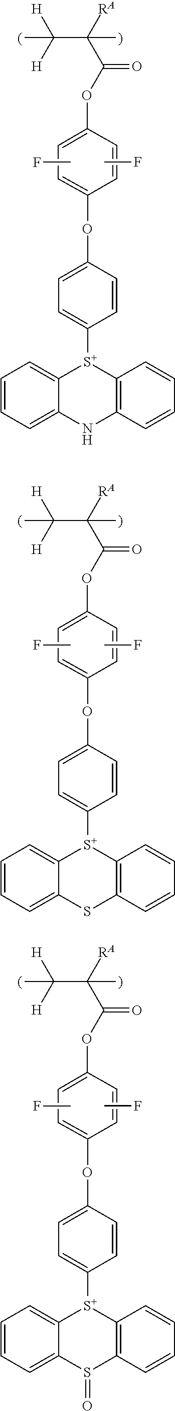

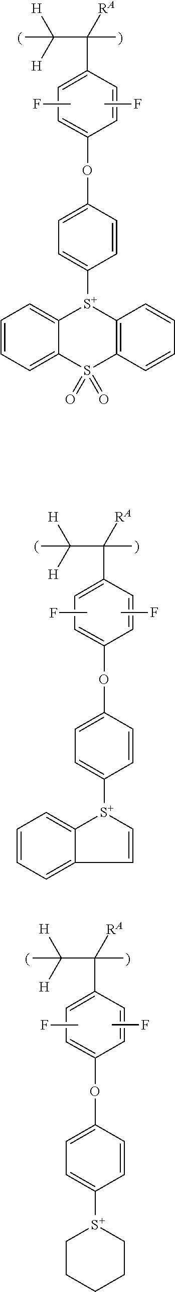

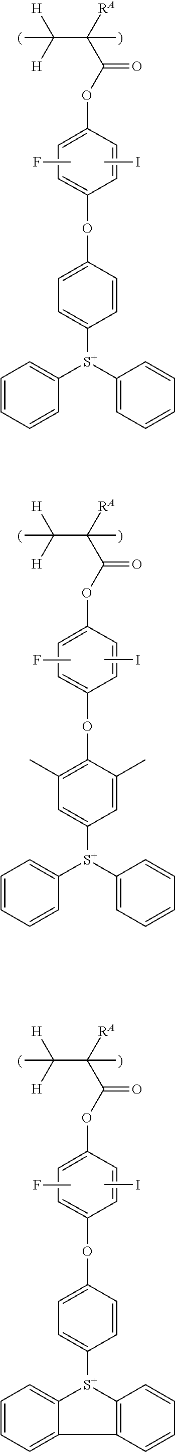

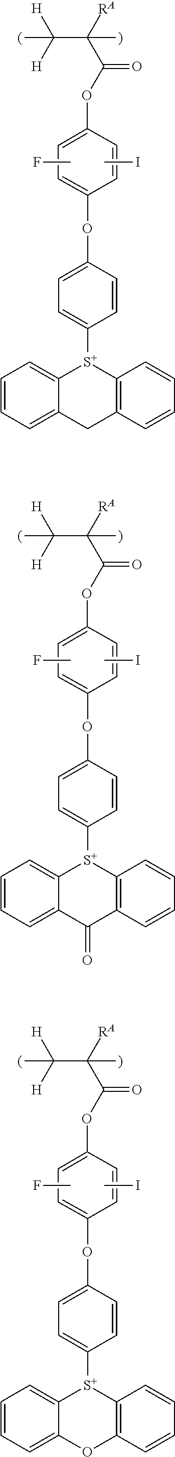

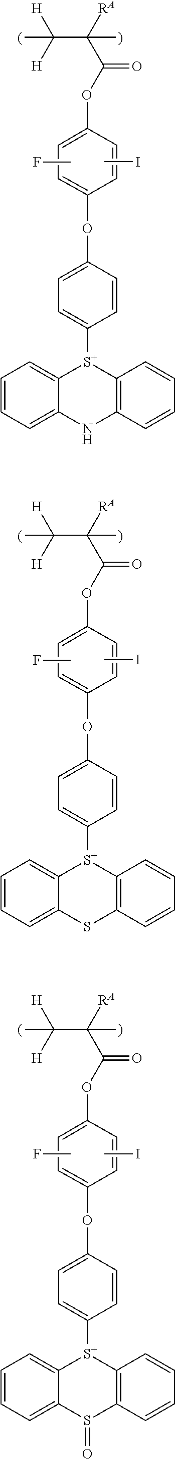

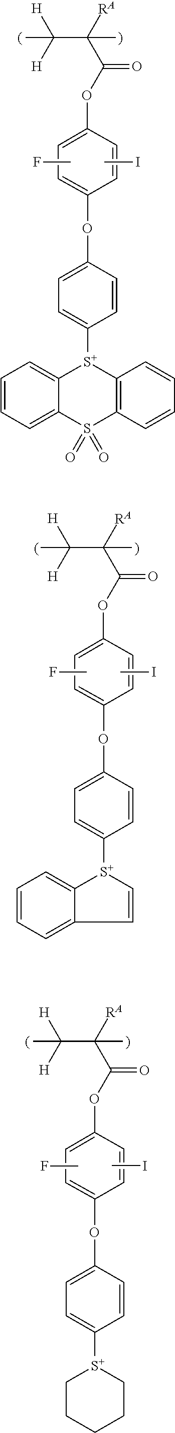

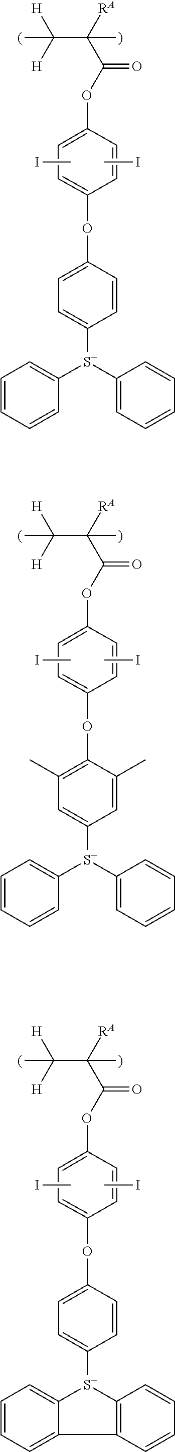

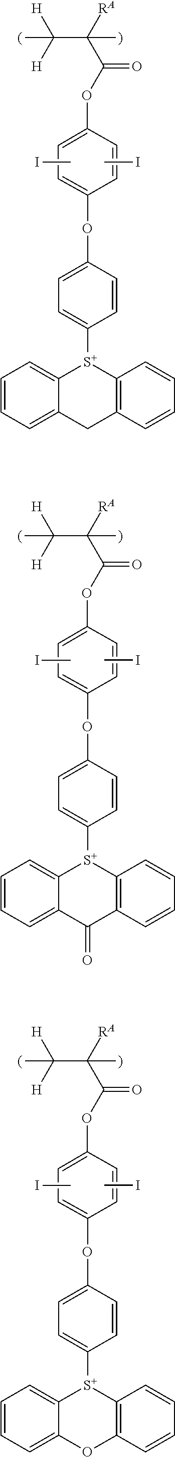

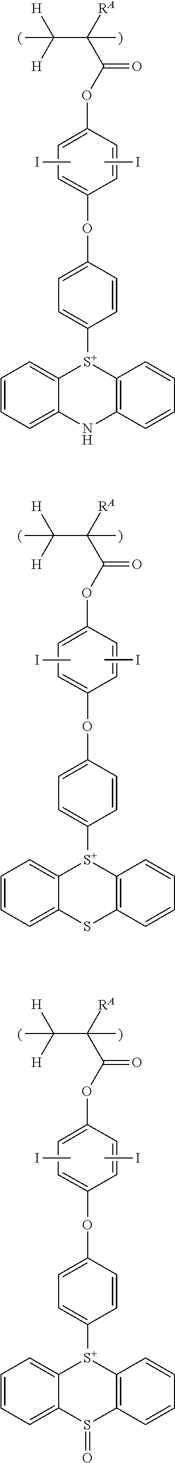

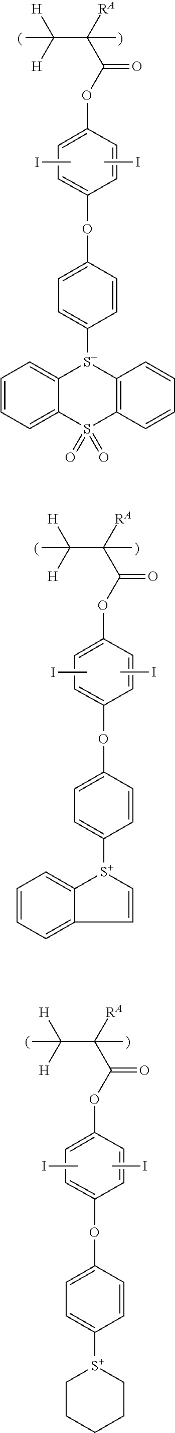

In the formula (c1), M⁻ is a non-nucleophilic counter ion. As non-nucleophilic counter ion, halide, sulfonate, imide and methide anions are preferred. Examples of the non-nucleophilic counter ion include halide ions such as chloride and bromide ions; sulfonate anions, specifically fluoroalkylsulfonate ions such as triflate, 1,1,1-trifluoroethanesulfonate, and nonafluorobutanesulfonate, arylsulfonate ions such as tosylate, benzenesulfonate, 4-fluorobenzenesulfonate, and 1,2,3,4,5-pentafluorobenzenesulfonate, alkylsulfonate ions such as mesylate and butanesulfonate; imide ions such as bis(trifluoromethylsulfonyl)imide, bis(perfluoroethylsulfonyl)imide, and bis(perfluorobutylsulfonyl)imide; and methide ions such as tris(trifluoromethylsulfonyl)methide, and tris(perfluoroethylsulfonyl)methide.

Anions having the formulae (c1-1) to (c1-4) are also useful as the non-nucleophilic counter ion.

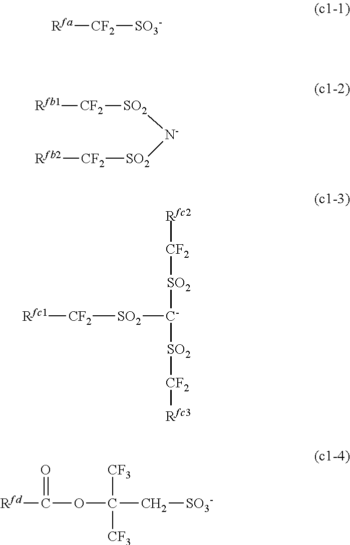

In the formula (c1-1), R^{fa} is fluorine or a C₁-C₄₀ hydrocarbyl group which may contain a heteroatom. The hydrocarbyl group may be saturated or unsaturated and straight, branched or cyclic. Examples thereof include those exemplified above for the hydrocarbyl group represented by R^{fa1} in the formula (c1-1-1).

As the anion having the formula (c1-1), an anion having the formula (c1-1-1) is preferred.

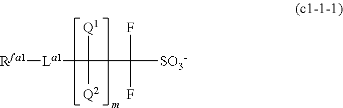

In the formula (c1-1-1), Q¹ and Q² are each independently hydrogen, fluorine, or a C₁-C₆ fluorinated saturated hydrocarbyl group. It is preferred for solvent solubility that at least one of Q¹ and Q² be trifluoromethyl. m is 0, 1, 2, 3, or 4, most preferably 1. R^{fa1} is a C₁-C₃₅ hydrocarbyl group which may contain a heteroatom. As the heteroatom, oxygen, nitrogen, sulfur and halogen atoms are preferred, with oxygen being more preferred. Of the hydrocarbyl groups, those groups of 6 to 30 carbon atoms are preferred from the aspect of achieving a high resolution in forming small-size patterns.

In (c1-1-1), the C₁-C₃₅ hydrocarbyl group represented by R^{fa1} may be saturated or unsaturated and straight, branched or cyclic. Examples thereof include C₁-C₃₅ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl; C₃-C₃₅ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl; C₂-C₃₅ unsaturated aliphatic hydrocarbyl groups such as 2-propenyl and 3-cyclohexenyl; C₆-C₃₅ aryl groups such as phenyl, 1-naphthyl, 2-naphthyl, and 9-fluorenyl; and C₇-C₃₅ aralkyl groups such as benzyl and diphenylmethyl; and combinations thereof.

In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, nitro moiety, carbonyl moiety, ether bond, ester bond, sulfonate ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl and 3-oxocyclohexyl groups.

In the formula (c1-1-1), L^{a1} is a single bond, ether bond, ester bond, sulfonate ester bond, carbonate bond or carbamate bond. From the aspect of synthesis, an ether bond or ester bond is preferred, with the ester bond being more preferred.

Examples of the anion having the formula (c1-1) include those shown below, but not limited thereto. In the formulae, Q¹ is as described above, and Ac is acetyl.

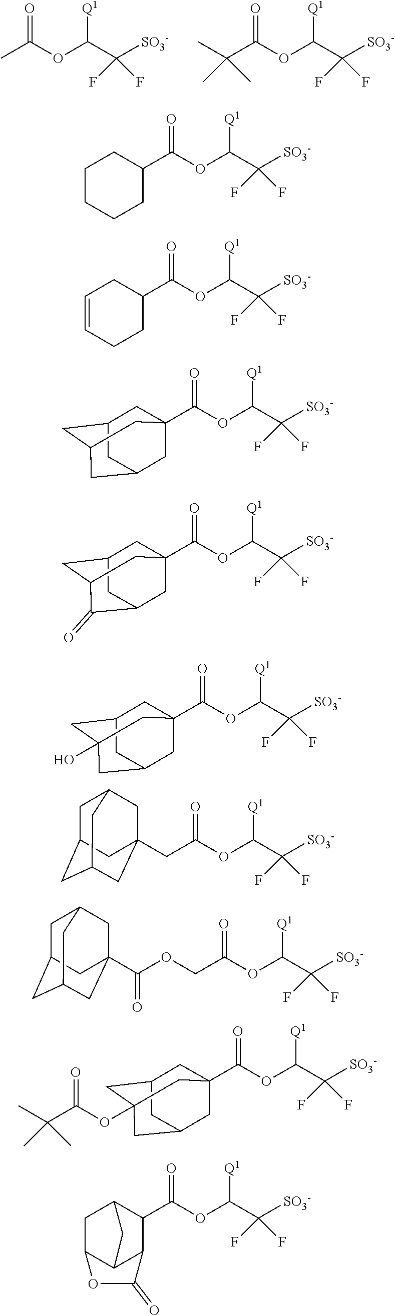

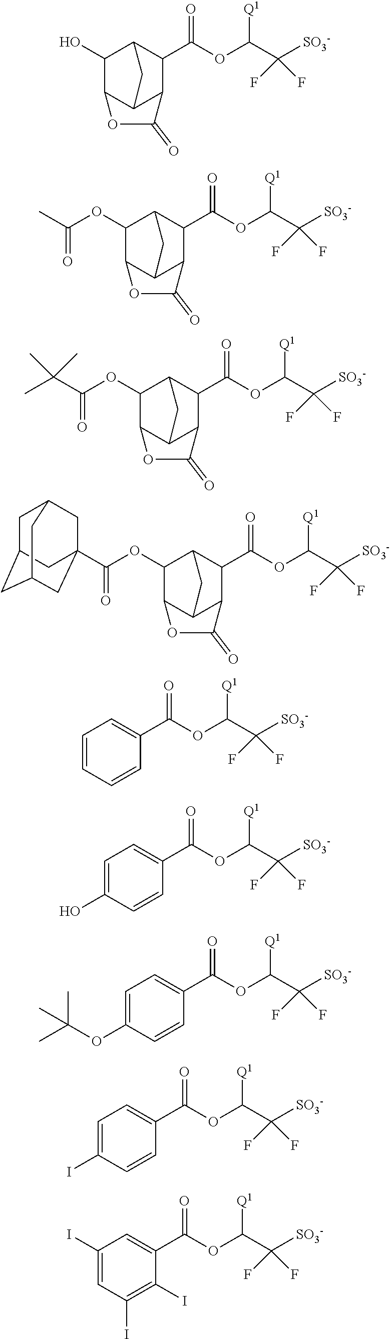

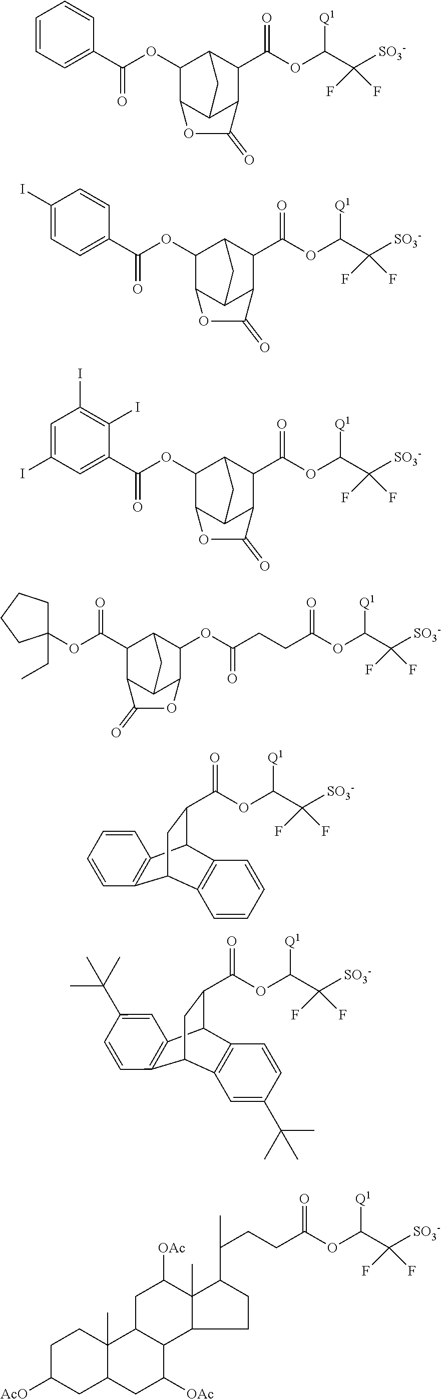

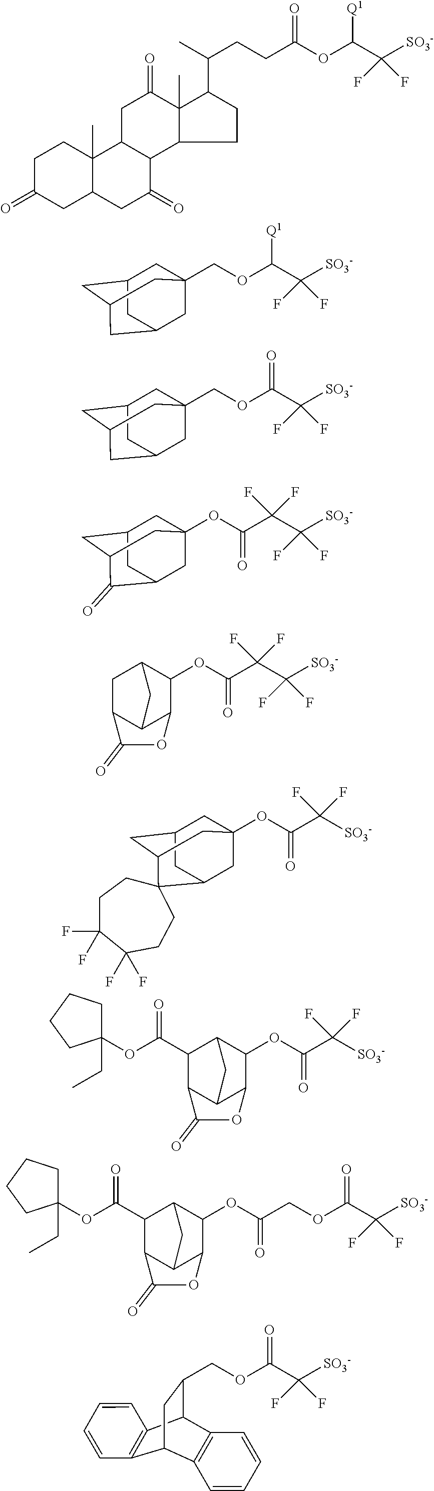

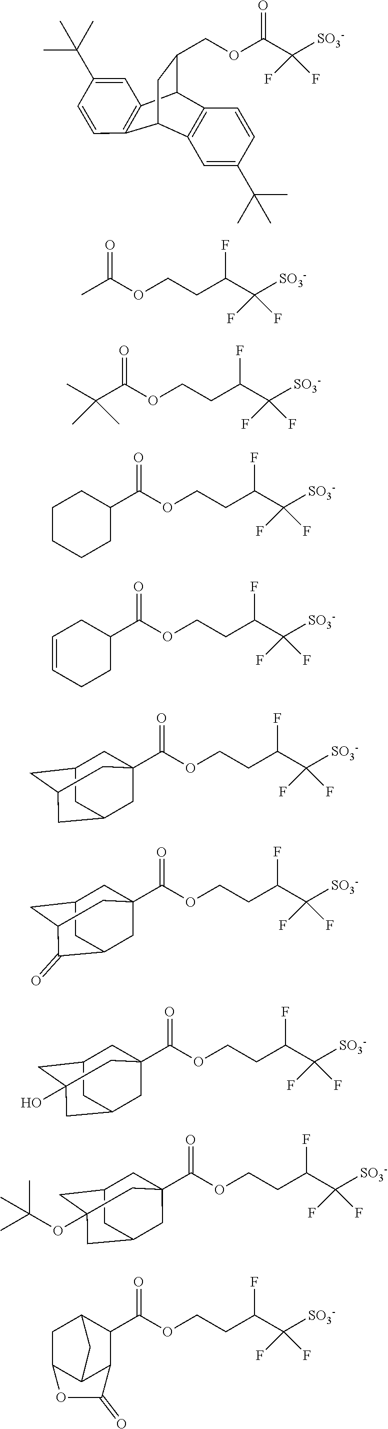

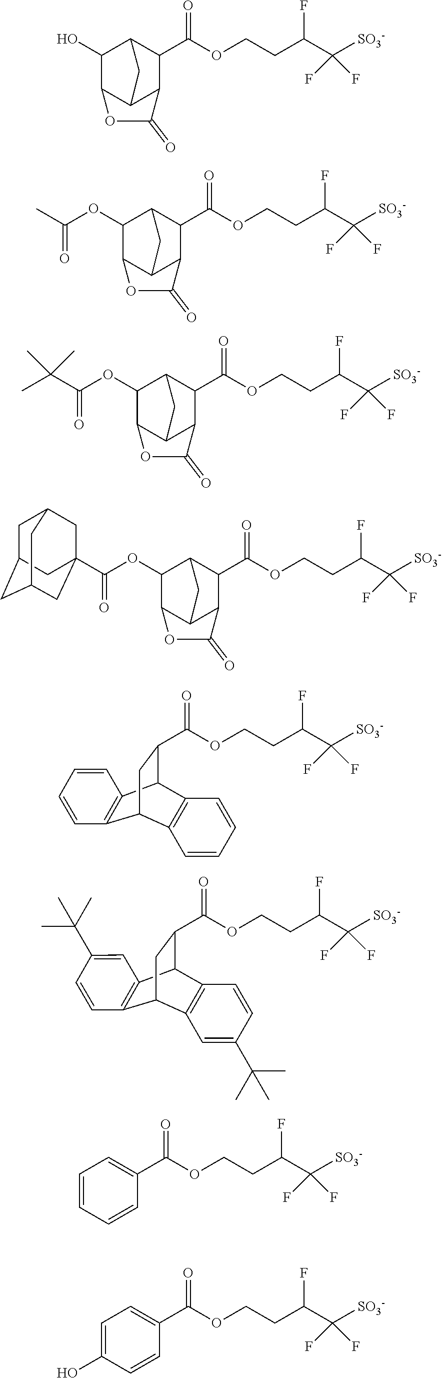

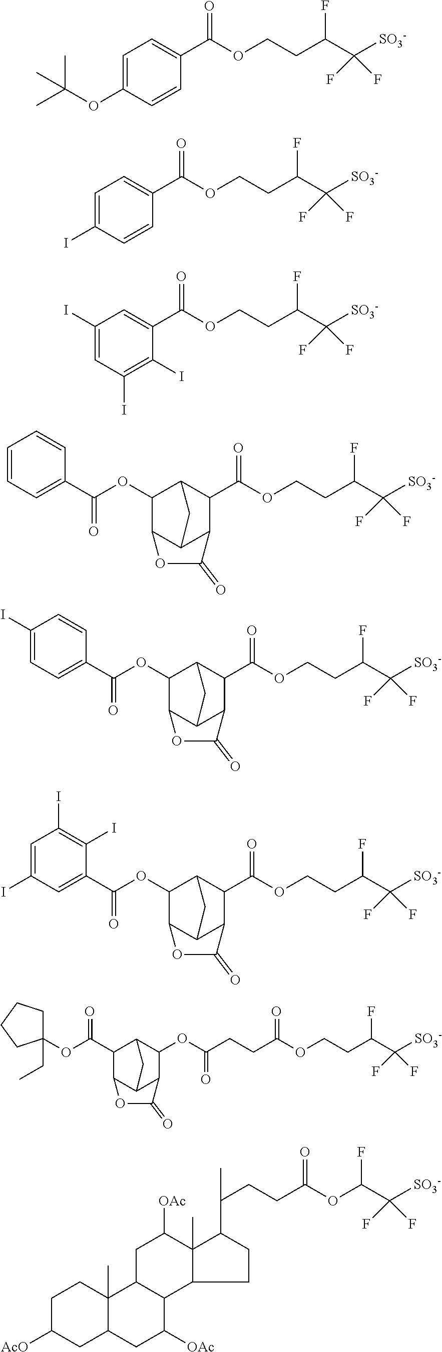

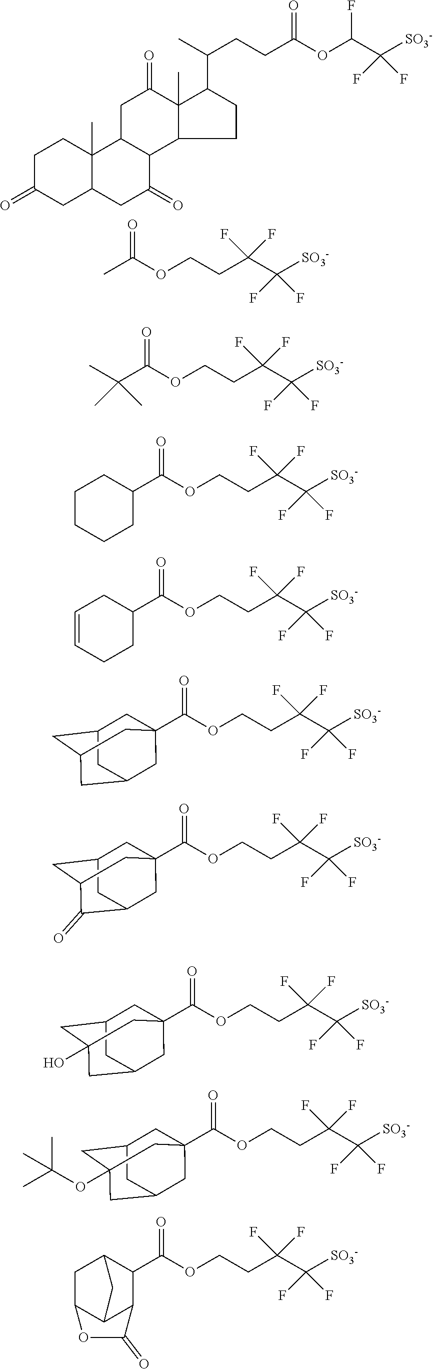

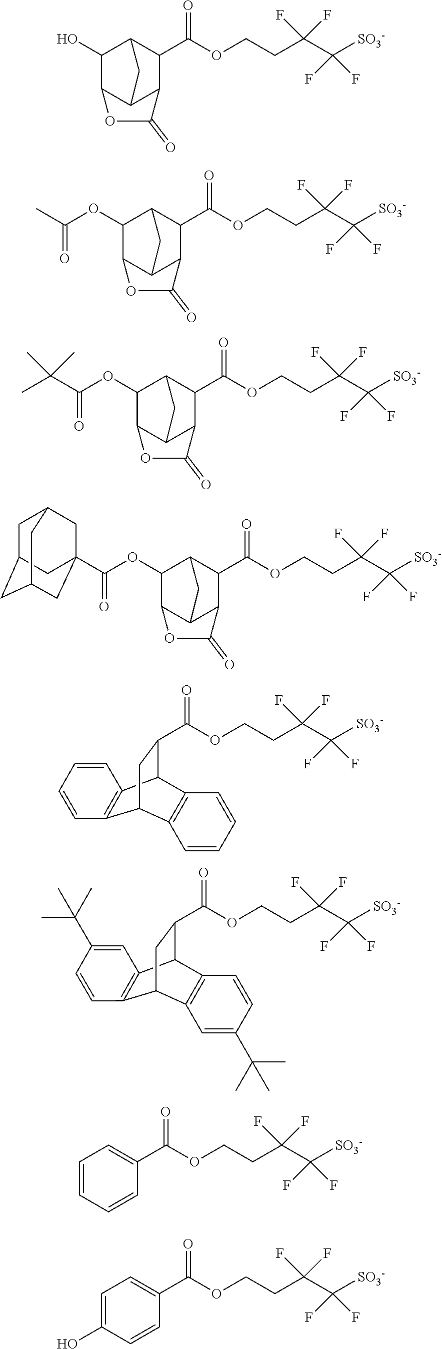

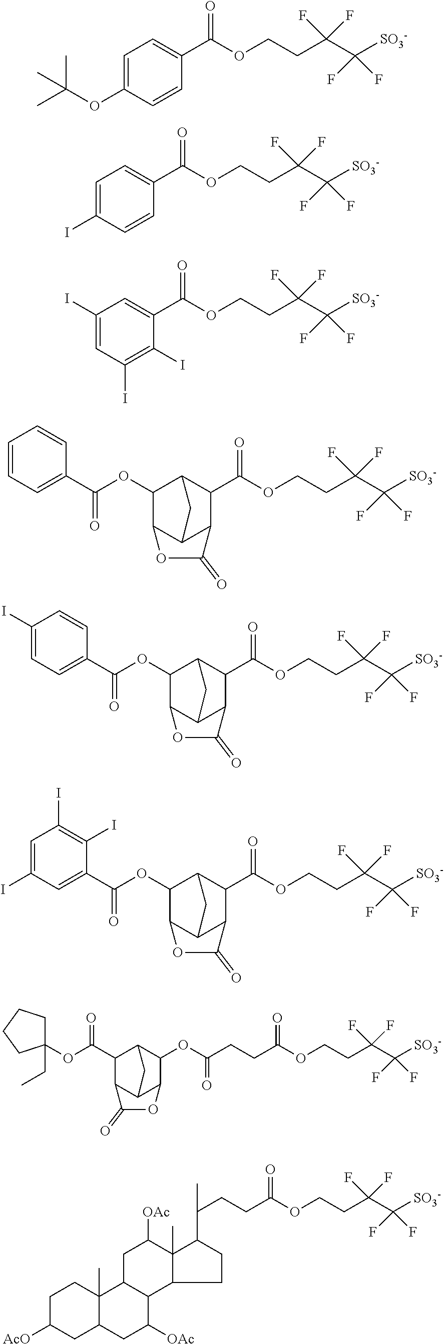

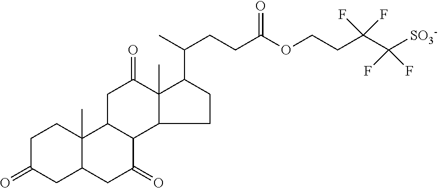

In the formula (c1-2), R^{fb1} and R^{fb2} are each independently fluorine or a C-4 hydrocarbyl group which may contain a heteroatom. The hydrocarbyl group may be saturated or unsaturated and straight, branched or cyclic. Examples thereof include those exemplified above for the hydrocarbyl group represented by R^{fa1} in the formula (c1-1-1). R^{fb1} and R^{fb2} are preferably fluorine or C₁-C₄ straight fluorinated alkyl groups. Also, R^{fb1} and R^{fb2} may bond together to form a ring with the linkage: —CF₂—SO₂—N—SO₂—CF₂— to which they are attached. It is preferred that a combination of R^{fb1} and R^{fb2} be a fluorinated ethylene or fluorinated propylene group.

In the formula (c1-3), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Examples thereof include those exemplified above for the hydrocarbyl group represented by R^{fa1} in the formula (c1-1-1). R^{fc1}, R^{fc2} and R^{fc3} are preferably fluorine or C₁-C₄ straight fluorinated alkyl groups. Also, R^{fc1} and R^{fc2} may bond together to form a ring with the linkage: —CF₂—SO₂—C⁻—SO₂—CF₂— to which they are attached. It is preferred that a combination of R^{fc1} and R^{fc2} be a fluorinated ethylene or fluorinated propylene group.

In the formula (c1-4), R^{fd} is a C₁-C₄₀ hydrocarbyl group which may contain a heteroatom. The hydrocarbyl group may be saturated or unsaturated and straight, branched or cyclic. Examples thereof include those exemplified above for the hydrocarbyl group represented by R^{fa1} in the formula (c1-1-1).

Examples of the anion having the formula (c1-4) include those shown below, but not limited thereto.

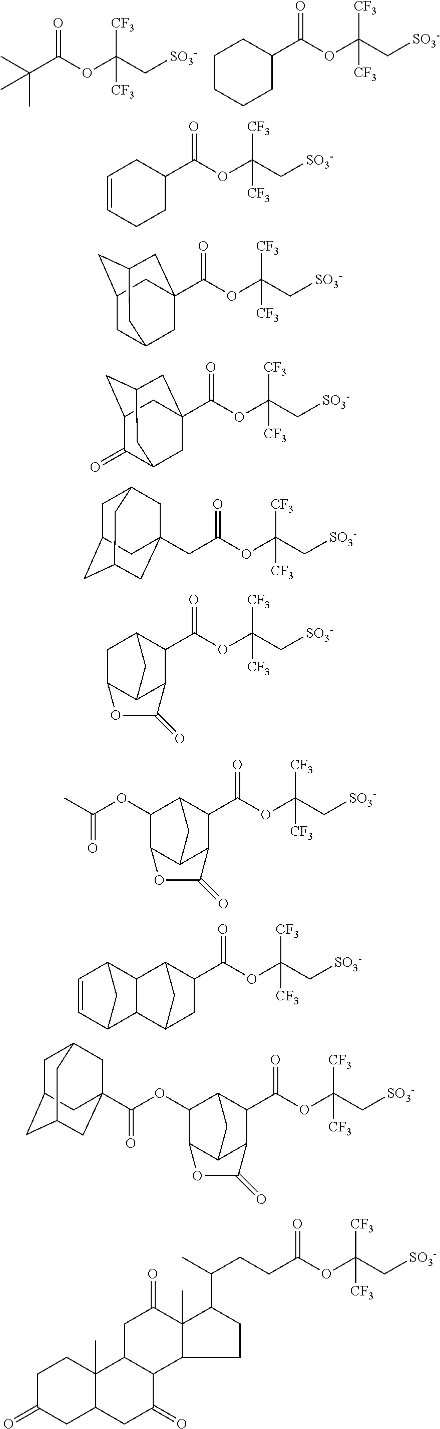

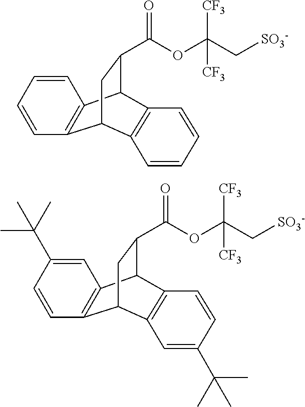

Examples of the non-nucleophilic counter ion further include anions having an aromatic ring substituted with iodine or bromine. Examples of the anions include anions having the formula (c1-5).

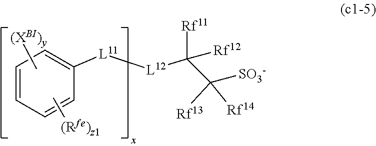

In the formula (c1-5), x1 is 1, 2 or 3. y1 is 1, 2, 3, 4, or 5. z1 is 0, 1, 2, or 3. It is noted that 1≤y1+z1≤5. y1 is preferably 1, 2, or 3, more preferably 2 or 3. z1 is preferably 0, 1, or 2.

In the formula (c1-5), X^{BI} is iodine or bromine. A plurality of X^{BI} may be identical or different when x1 and/or y1 is 2 or more.

In the formula (c1-5), L¹¹ is a single bond, ether bond, ester bond, or a C₁-C₆ saturated hydrocarbylene group which may contain an ether bond or ester bond. The saturated hydrocarbylene group may be straight, branched or cyclic.

In the formula (c1-5), L¹² is a single bond or a C₁-C₂₀ divalent linking group when x1=1, or a C₁-C₂₀ (x1+1)-valent linking group when x1=2 or 3. The linking group may contain an oxygen, sulfur or nitrogen atom.

In the formula (c1-5), R^{fe} is hydroxy, carboxy, fluorine, chlorine, bromine, amino group, or a C₁-C₂₀ hydrocarbyl, C1-C₂₀ hydrocarbyloxy, C₂-C₂₀ hydrocarbylcarbonyl, C₂-C₂₀ hydrocarbyloxycarbonyl, C₂-C₂₀ hydrocarbylcarbonyloxy, or C₁-C₂₀ hydrocarbylsulfonyloxy group, which may contain fluorine, chlorine, bromine, a hydroxy moiety, amino moiety or ether bond, or —N(R^{feA})(R^{feB}), —N(R^{feC})—C(═O)—R^{feD} or —N(R^{feC})—C(═O)—O—R^{feD}. R^{feA} and R^{feB} are each independently hydrogen or a C₁-C₆ saturated hydrocarbyl group. R^{feC} is hydrogen, or a C₁-C₆ saturated hydrocarbyl group which may contain halogen, a hydroxy, C₁-C₆ saturated hydrocarbyloxy, C₂-C₆ saturated hydrocarbylcarbonyl or C₂-C₆ saturated hydrocarbylcarbonyloxy moiety. R^{feD} is a C₁-C₁₆ aliphatic hydrocarbyl group, C₆-C₁₂ aryl group or C₇-C₁₅ aralkyl group, which may contain halogen, a hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy, and hydrocarbylsulfonyloxy groups may be straight, branched or cyclic. A plurality of R^{fe} may be identical or different when x1 and/or z1 is 2 or more.

Of these, R^{fe} is preferably hydroxy, —N(R^{feC})—C(═O)—R^{feD}, —N(R^{feC})—C(═O)—O—R^{feD}, fluorine, chlorine, bromine, methyl or methoxy.

In the formula (c1-5), Rf¹¹ to Rf¹⁴ are each independently hydrogen, fluorine or trifluoromethyl, and at least one of Rf¹¹ to Rf¹⁴ is fluorine or trifluoromethyl. Rf¹¹ and Rf¹², taken together, may form a carbonyl group. More preferably, both Rf¹³ and Rf¹⁴ are fluorine.

Examples of the anion having the formula (c1-5) include those shown below, but not limited thereto. In the formulae, X^{BI} is as defined above.

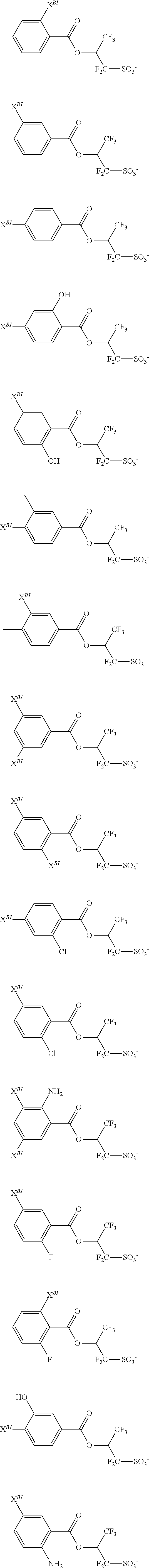

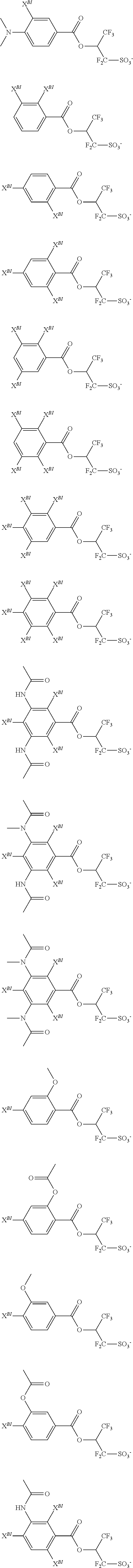

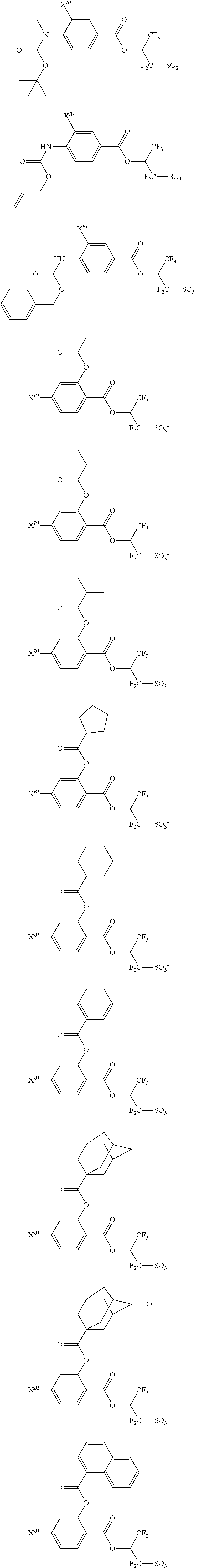

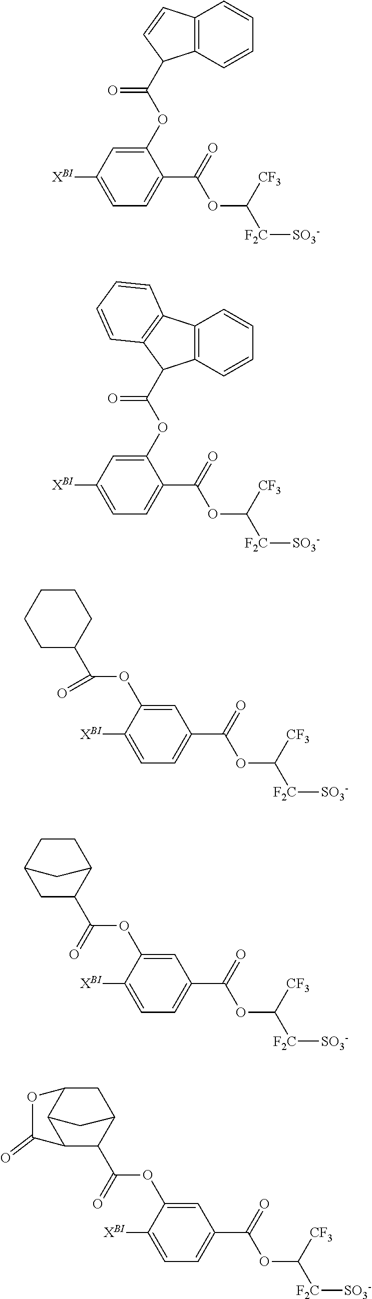

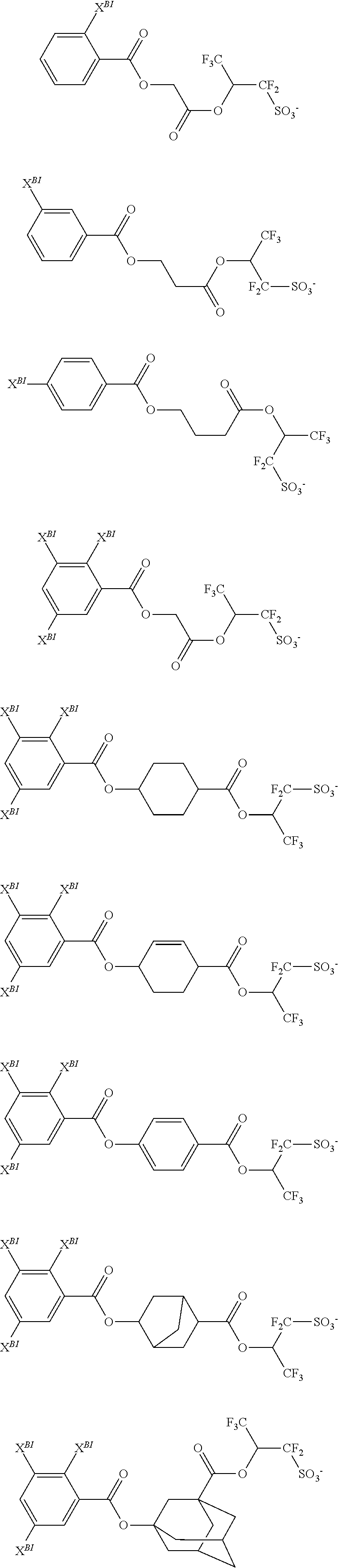

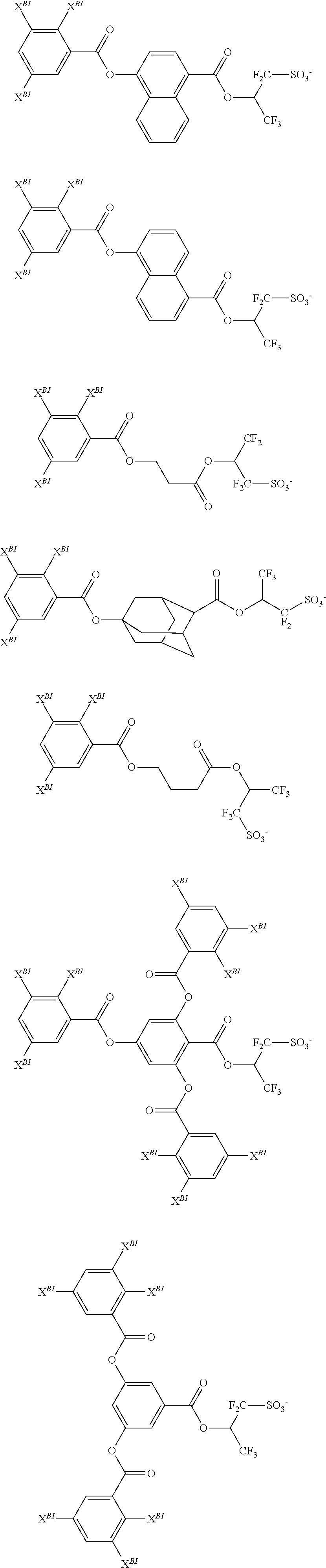

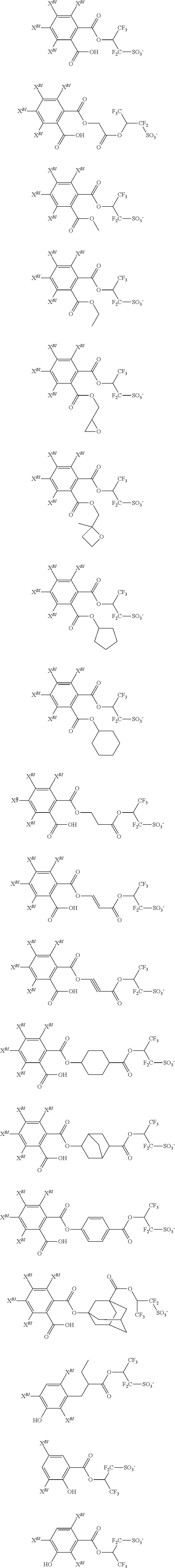

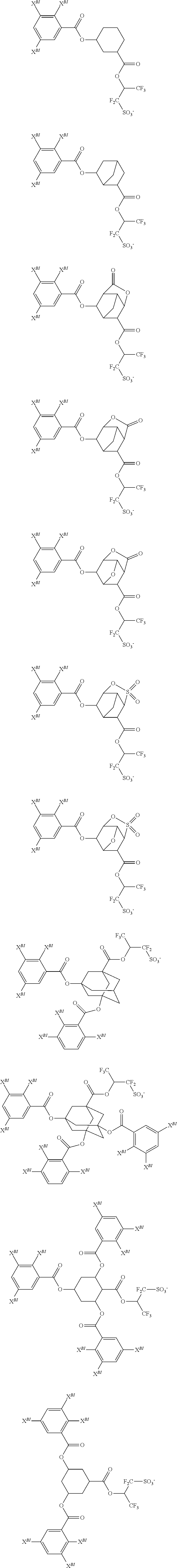

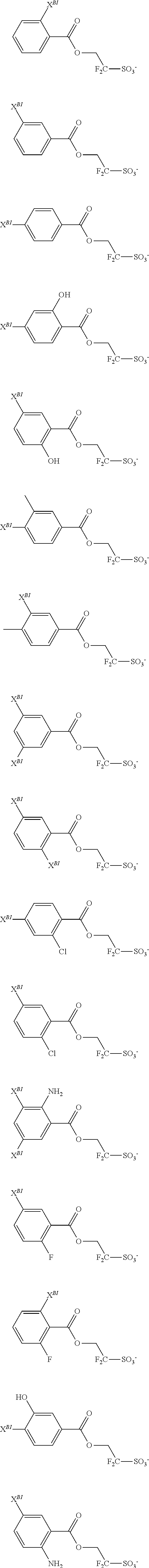

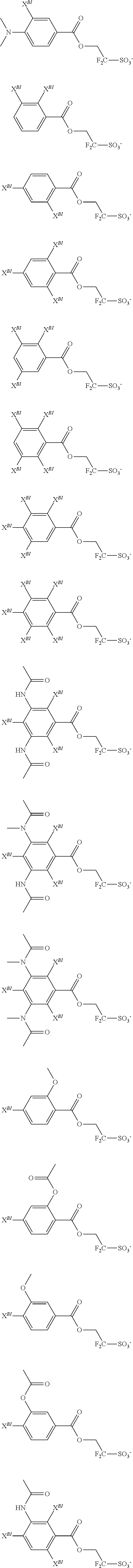

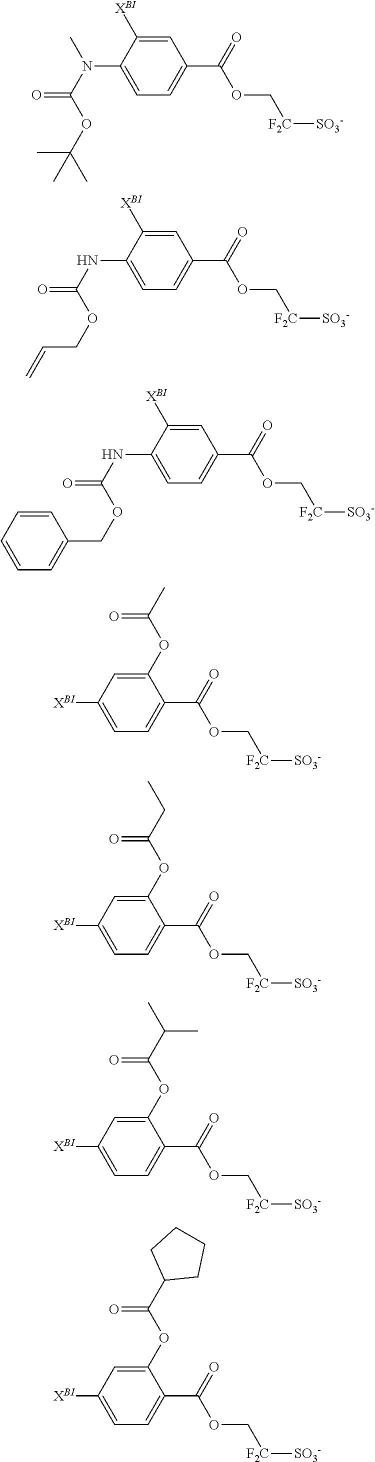

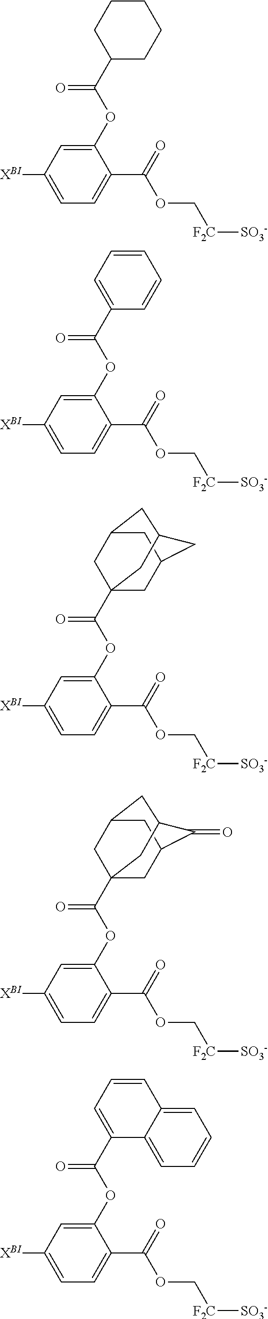

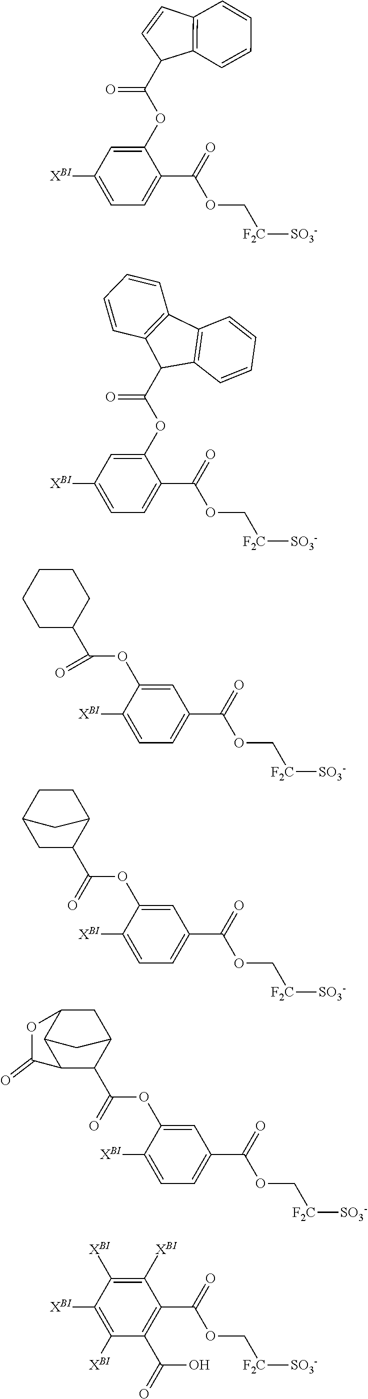

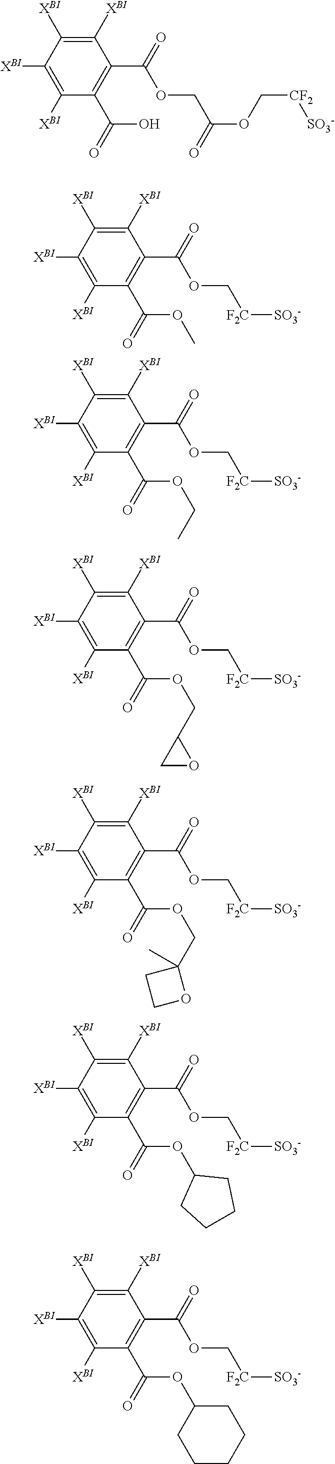

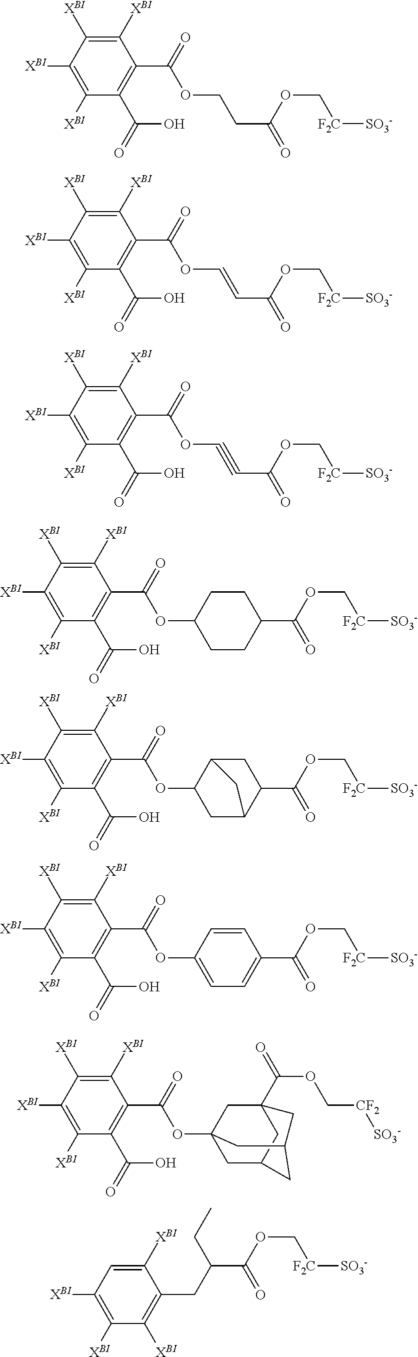

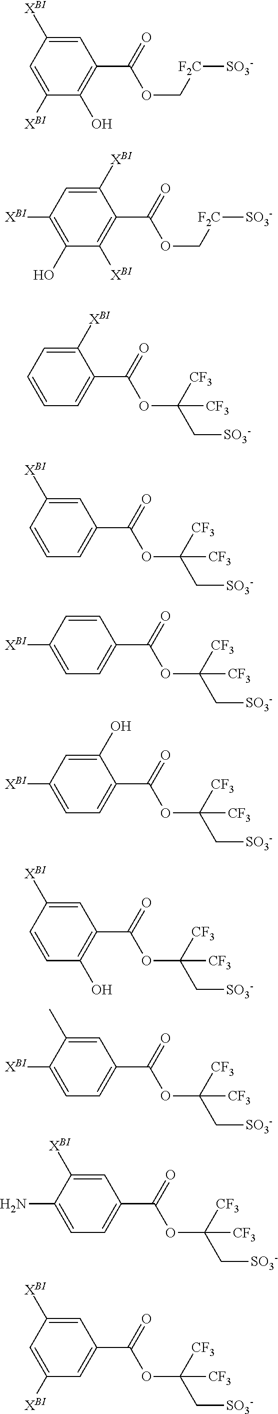

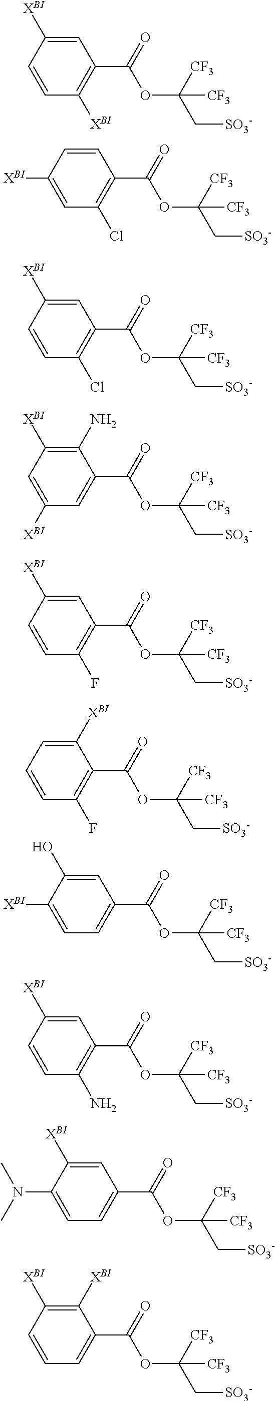

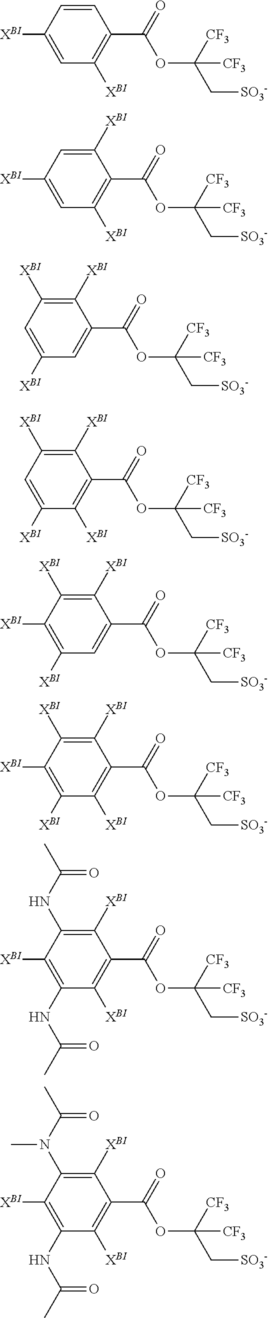

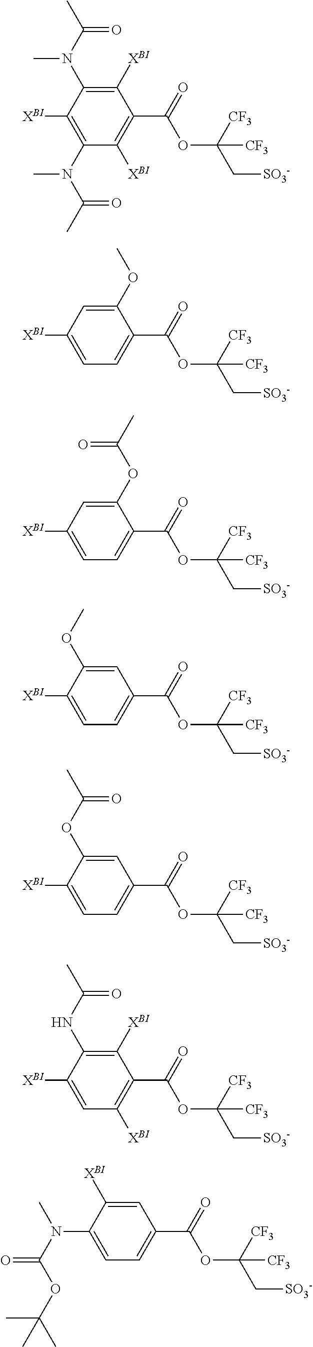

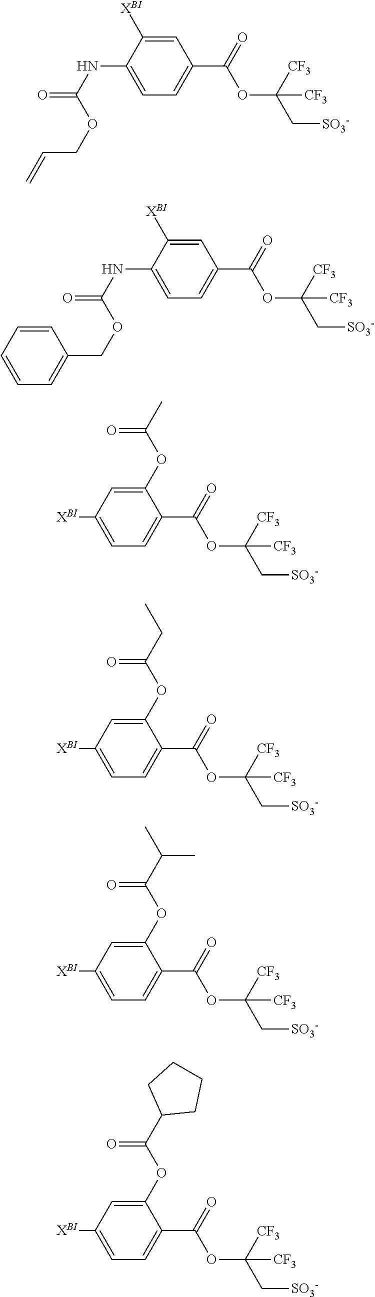

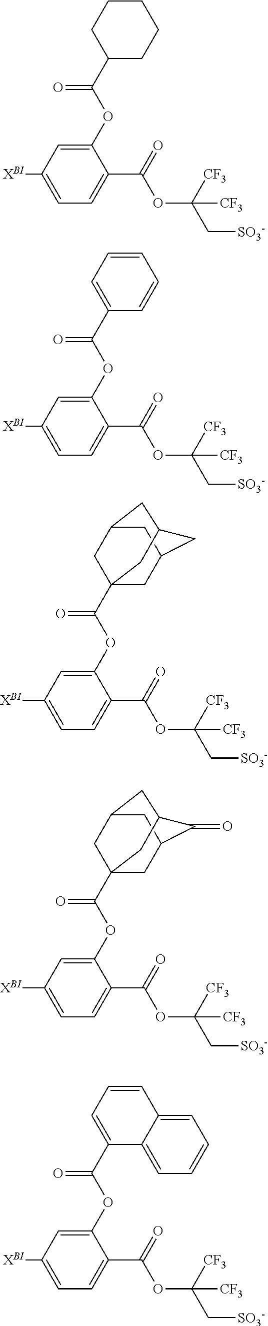

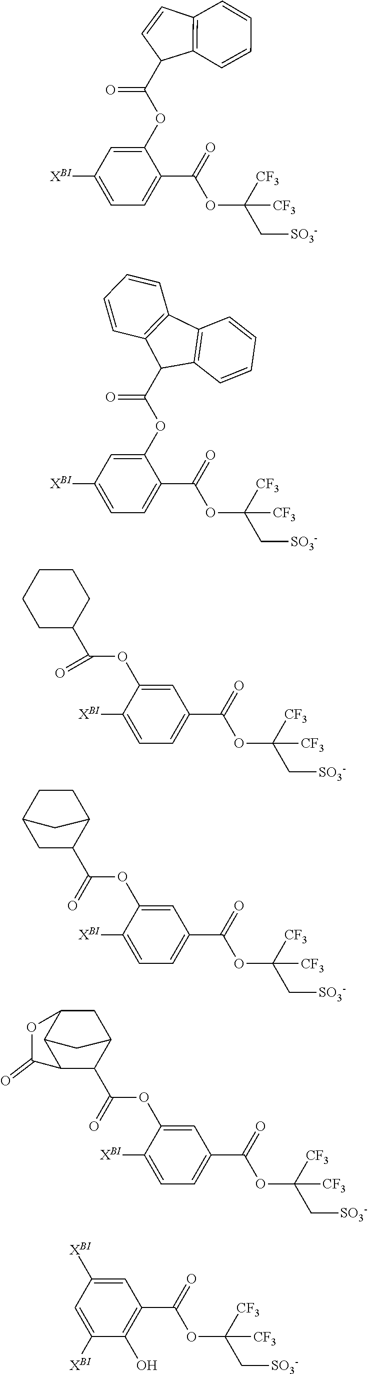

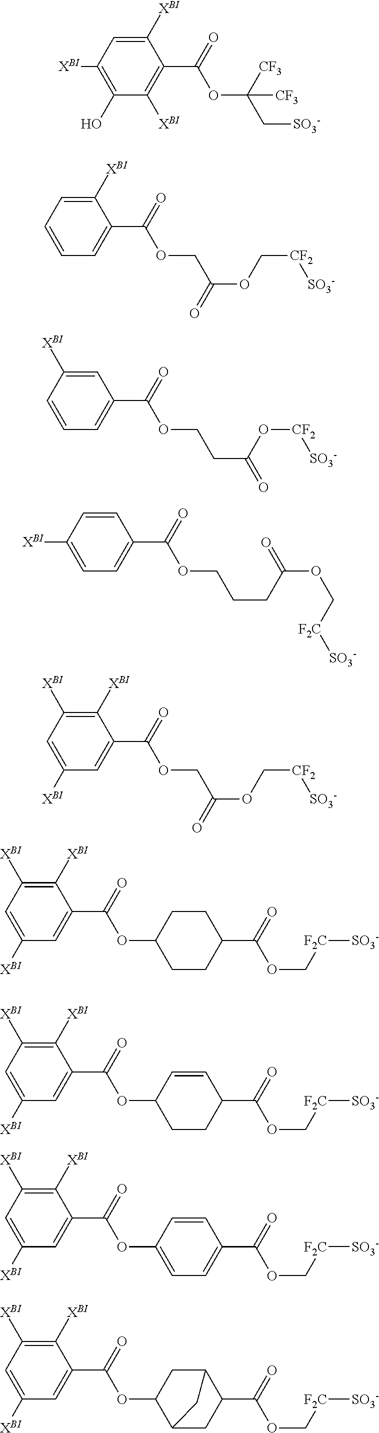

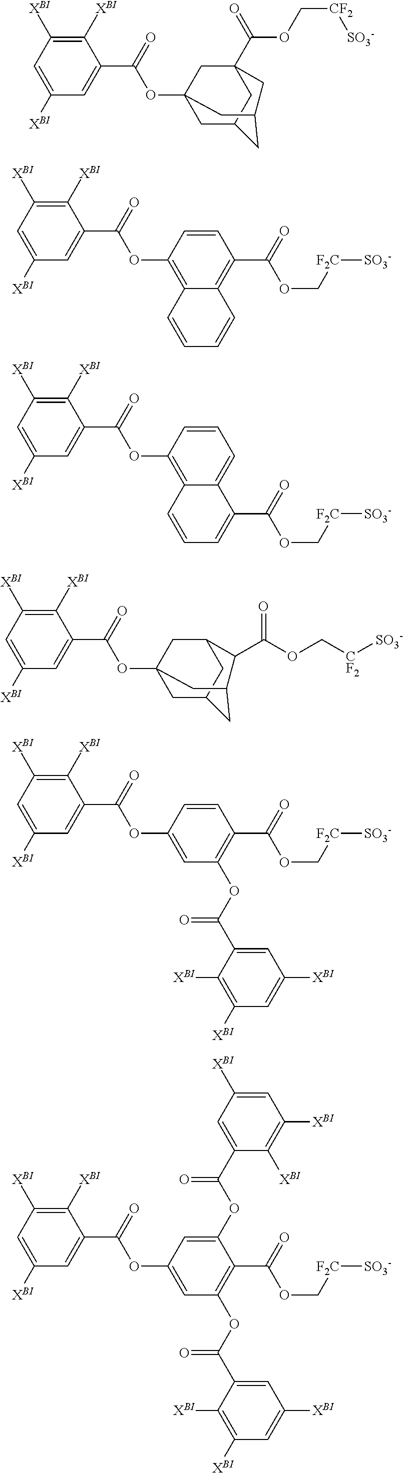

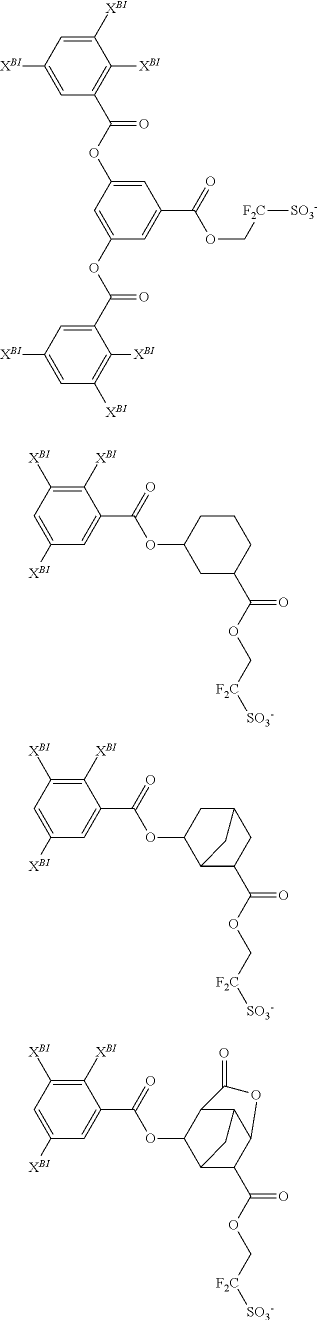

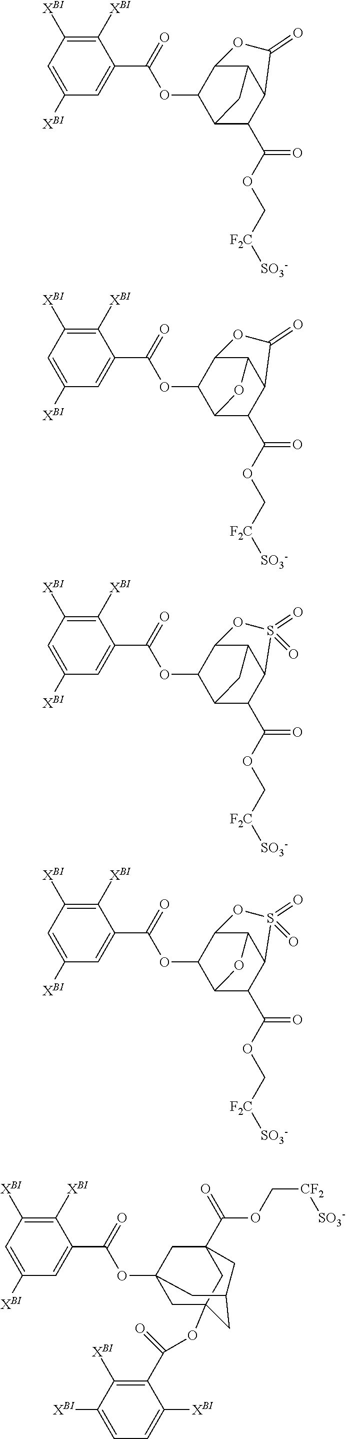

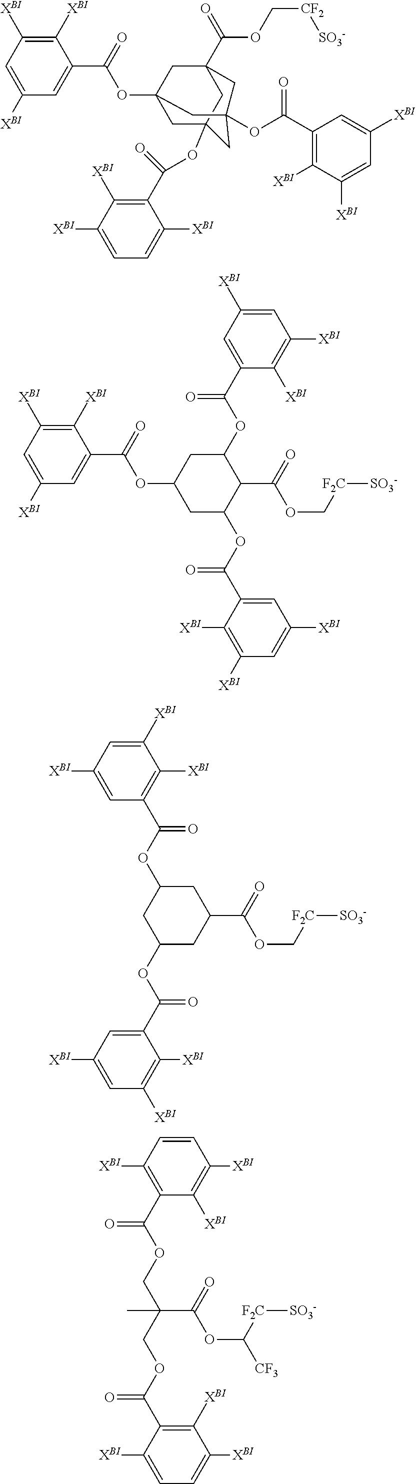

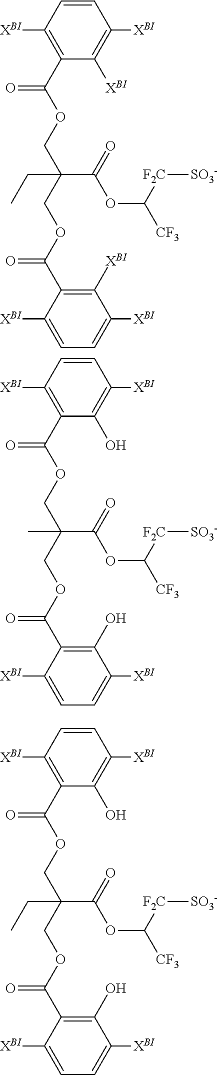

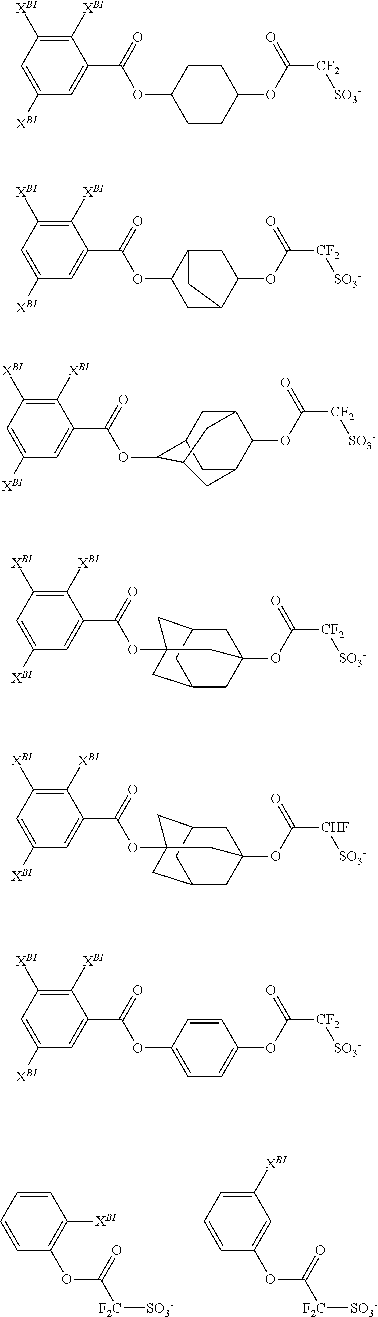

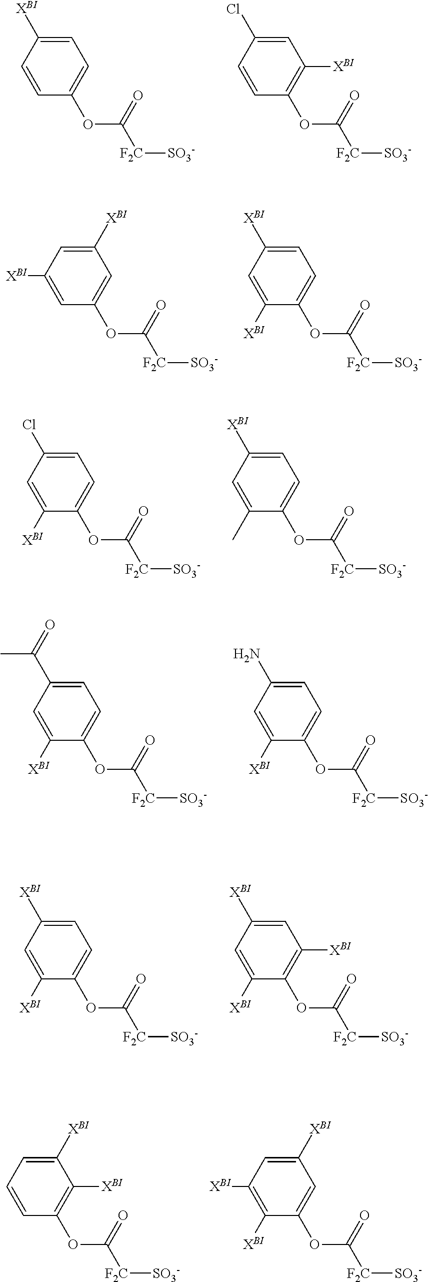

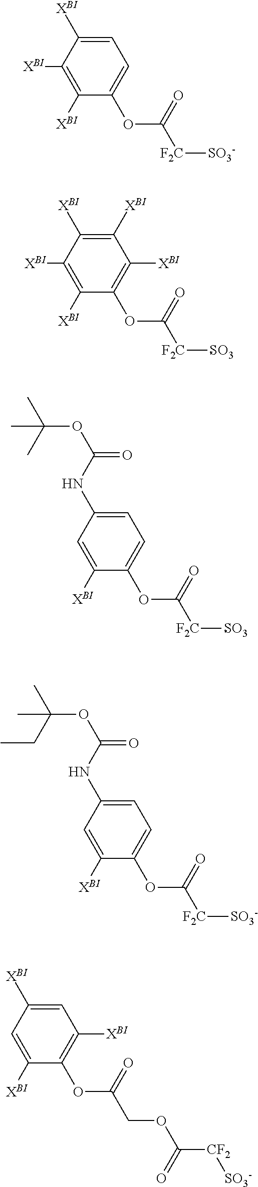

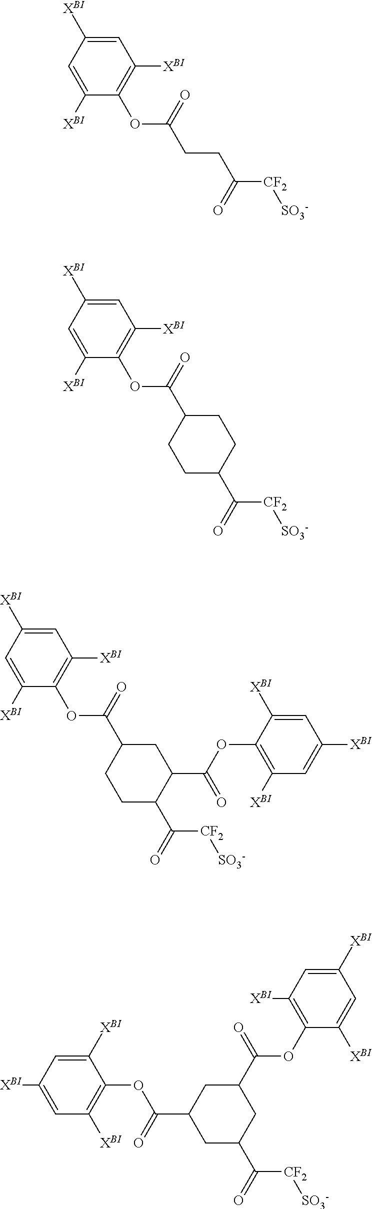

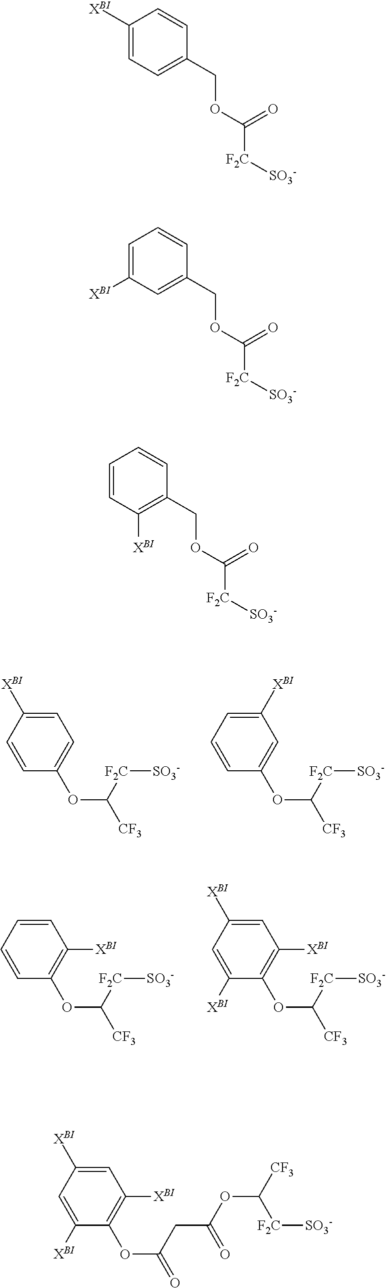

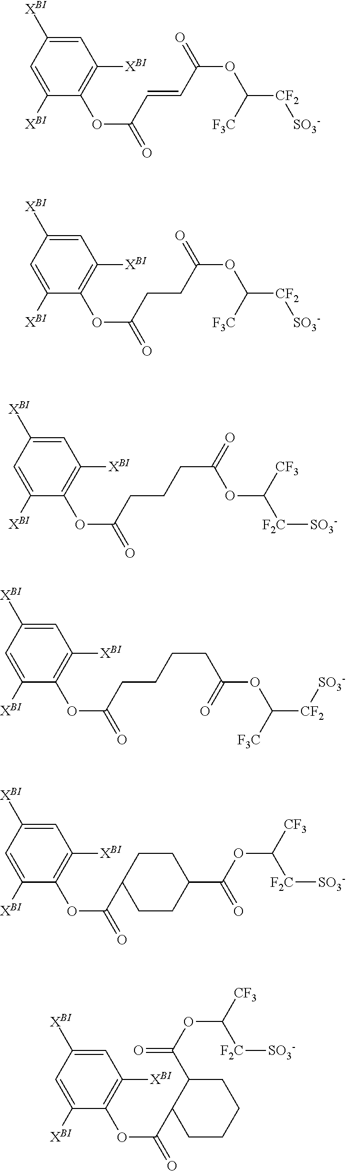

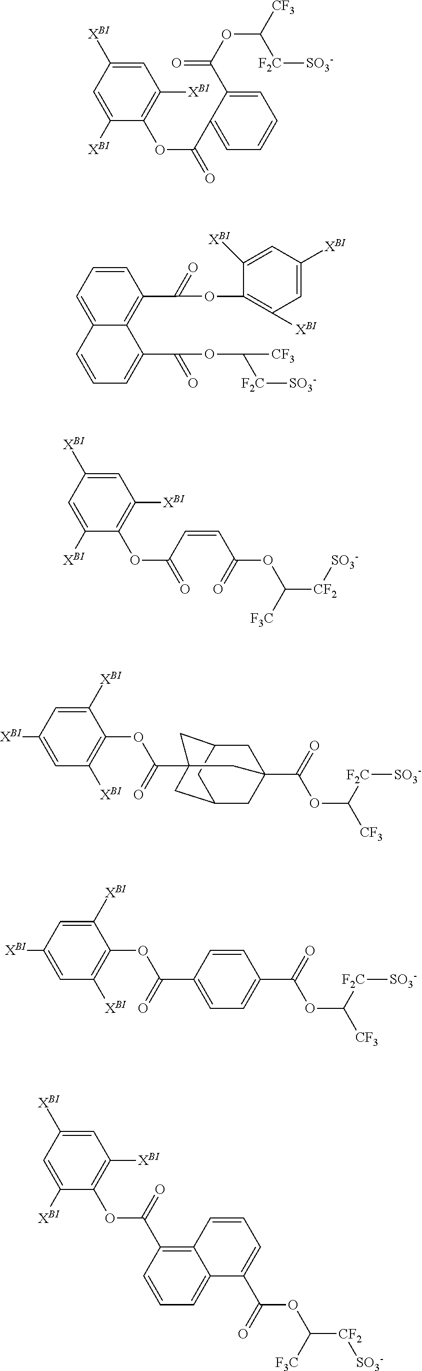

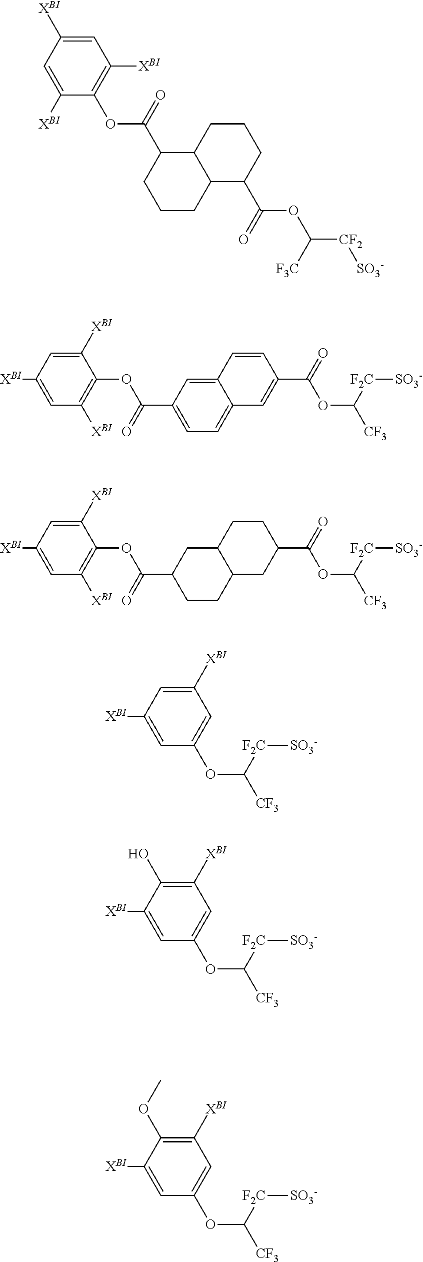

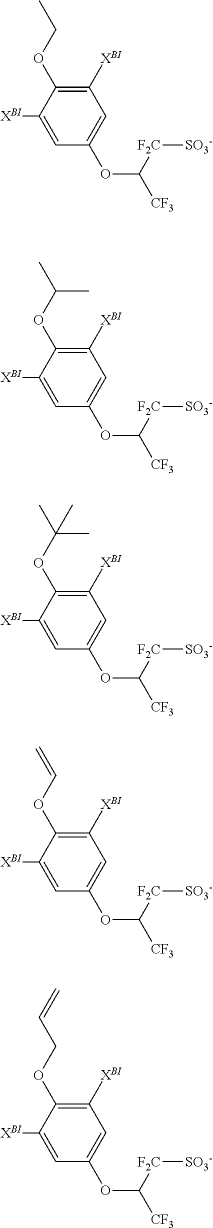

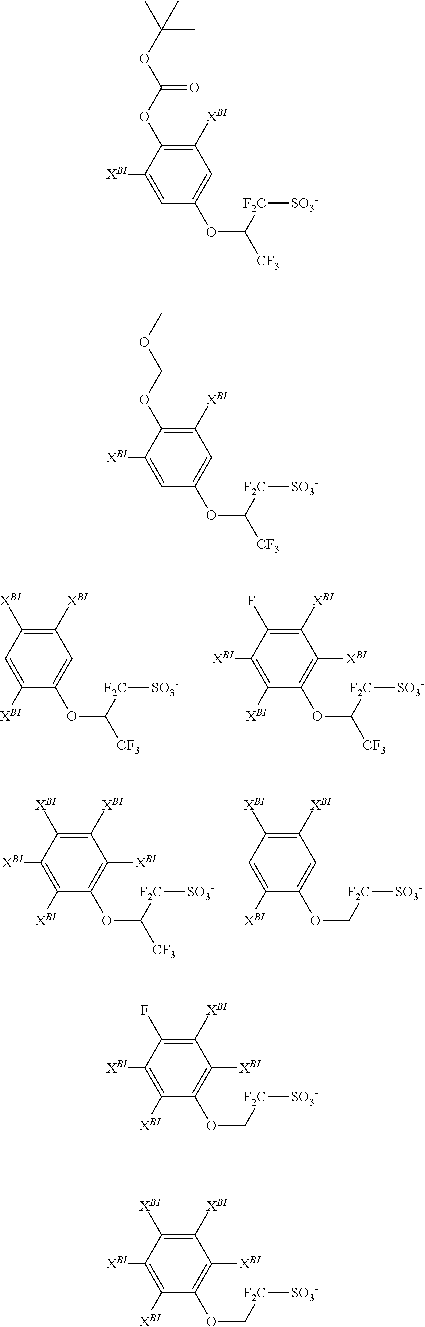

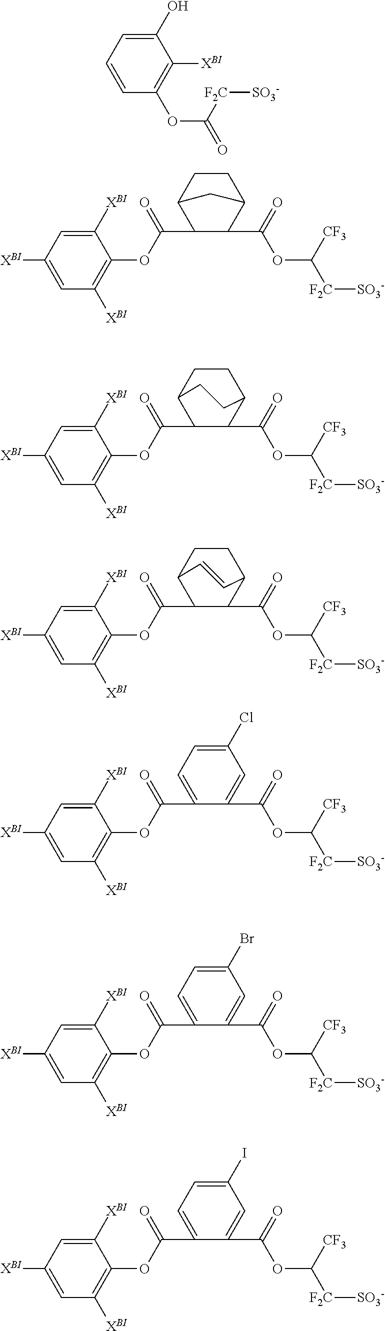

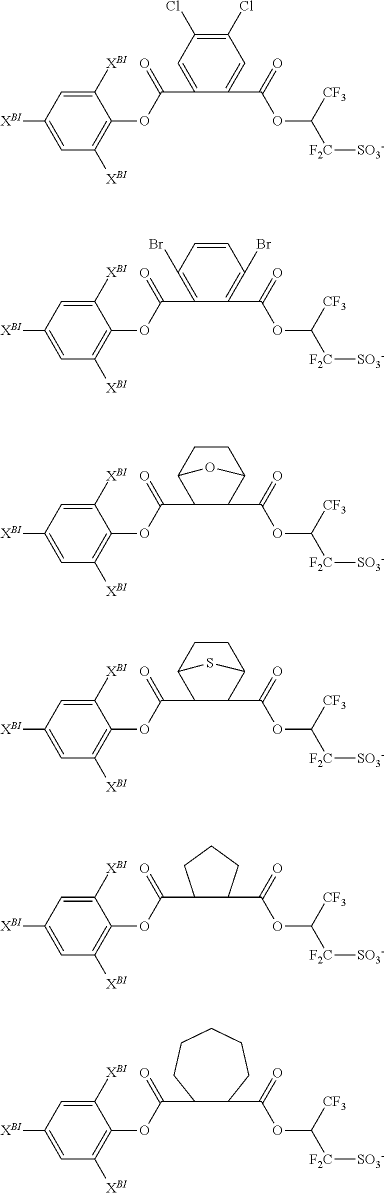

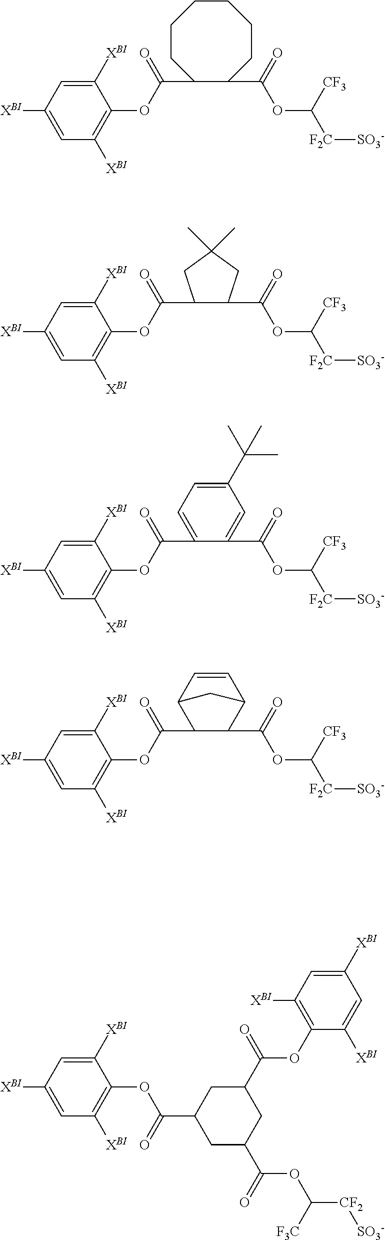

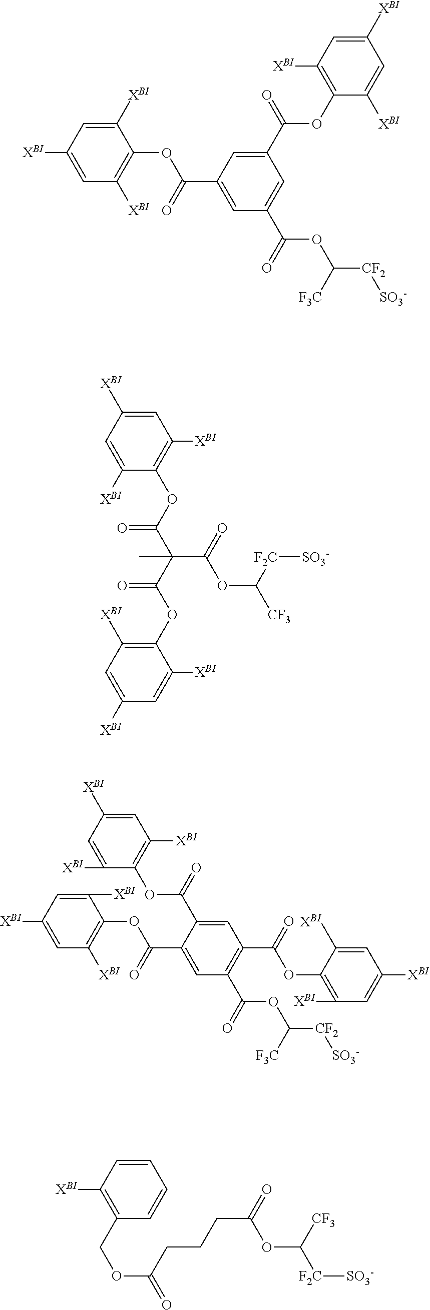

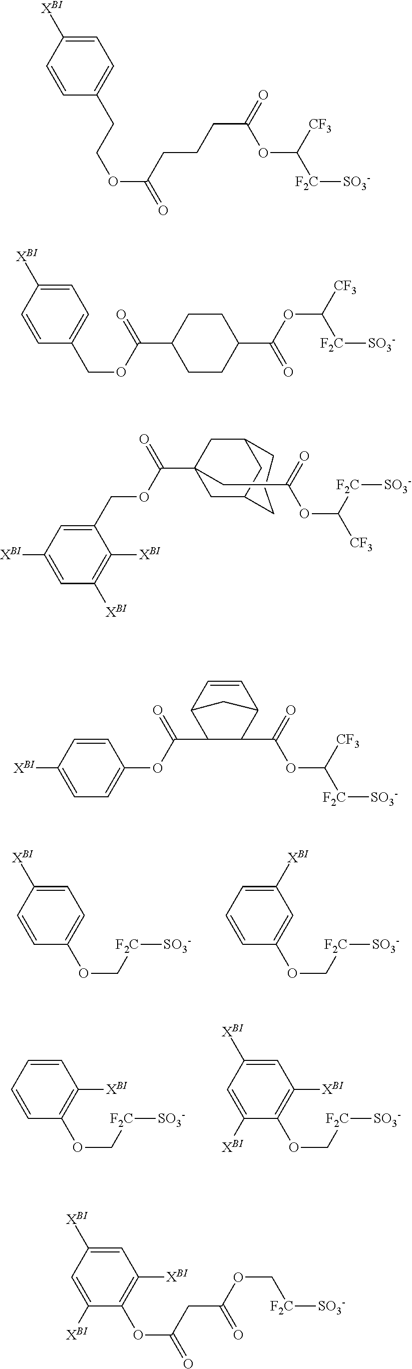

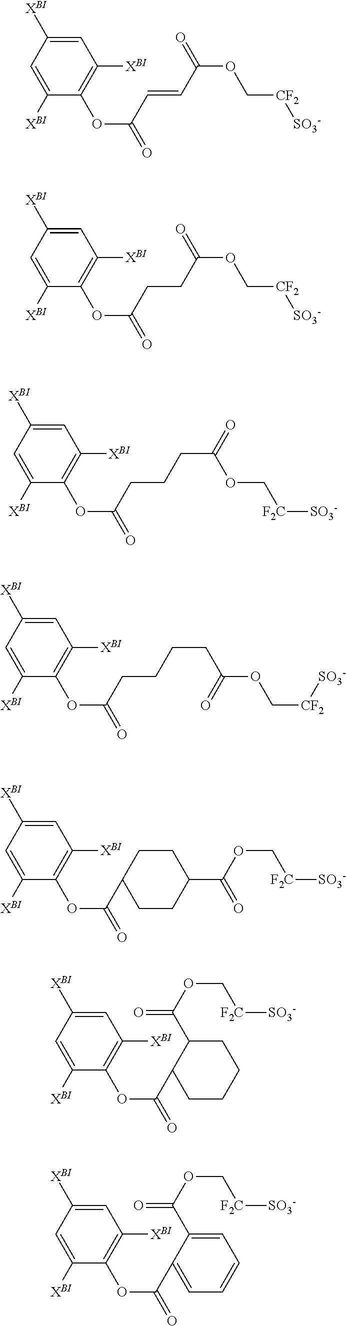

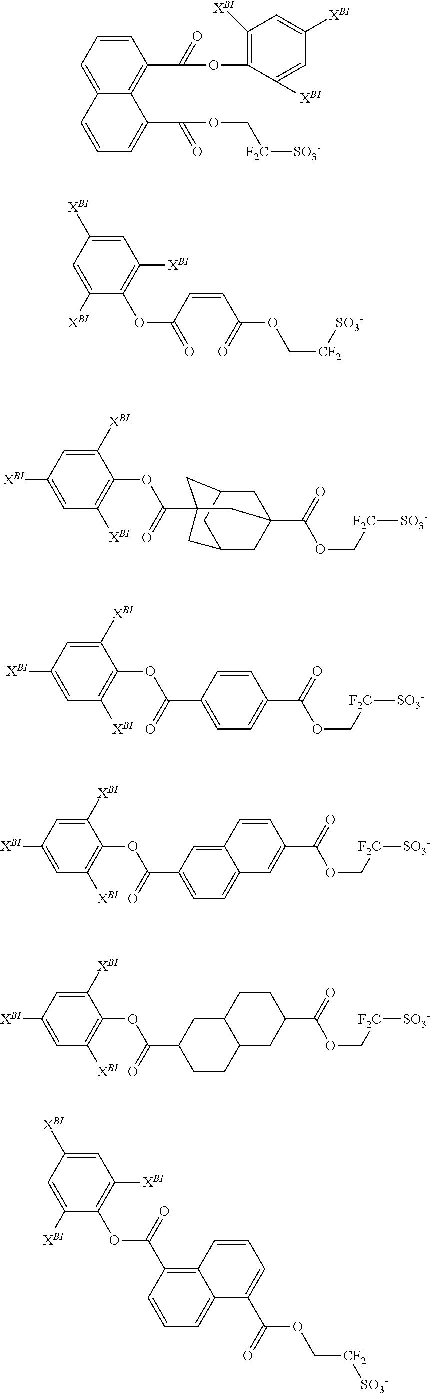

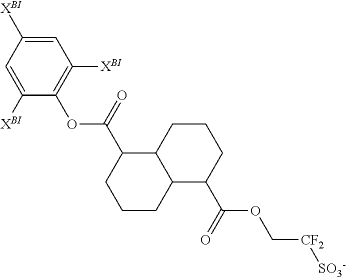

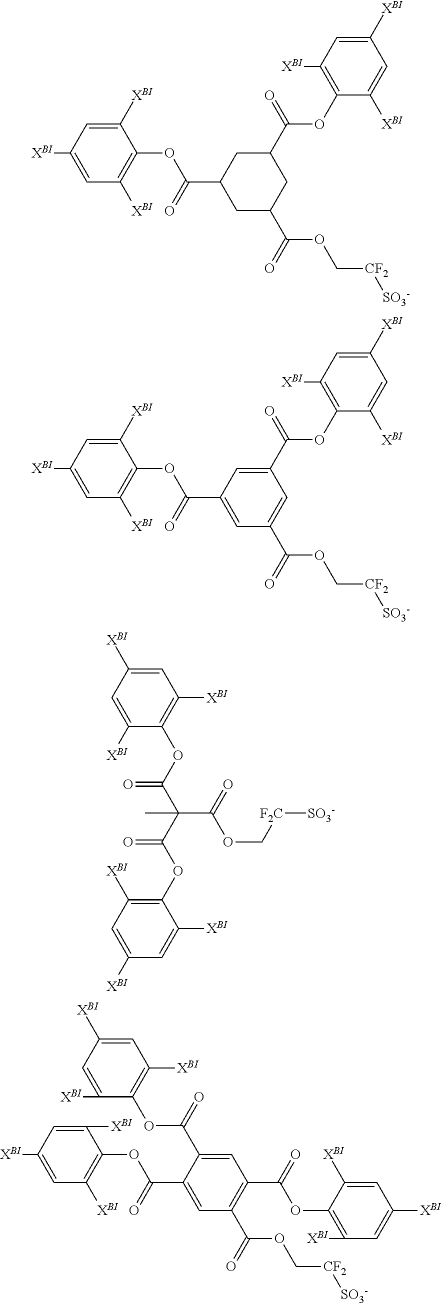

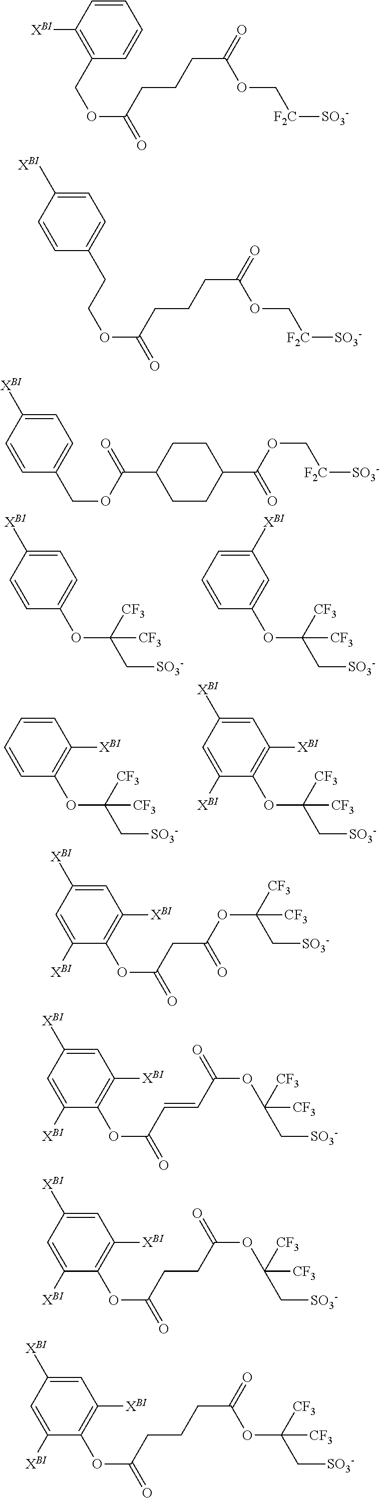

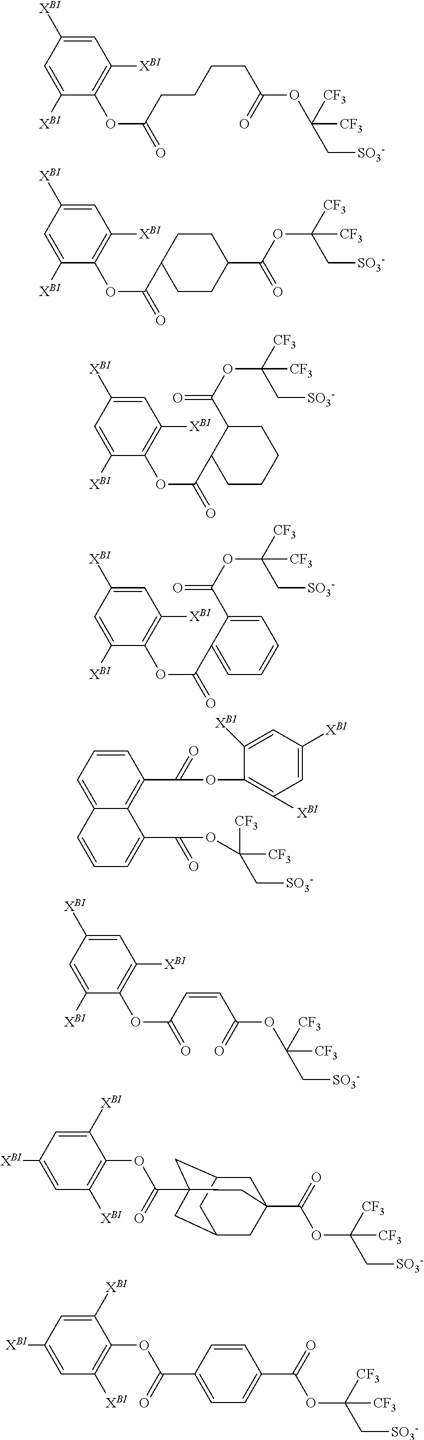

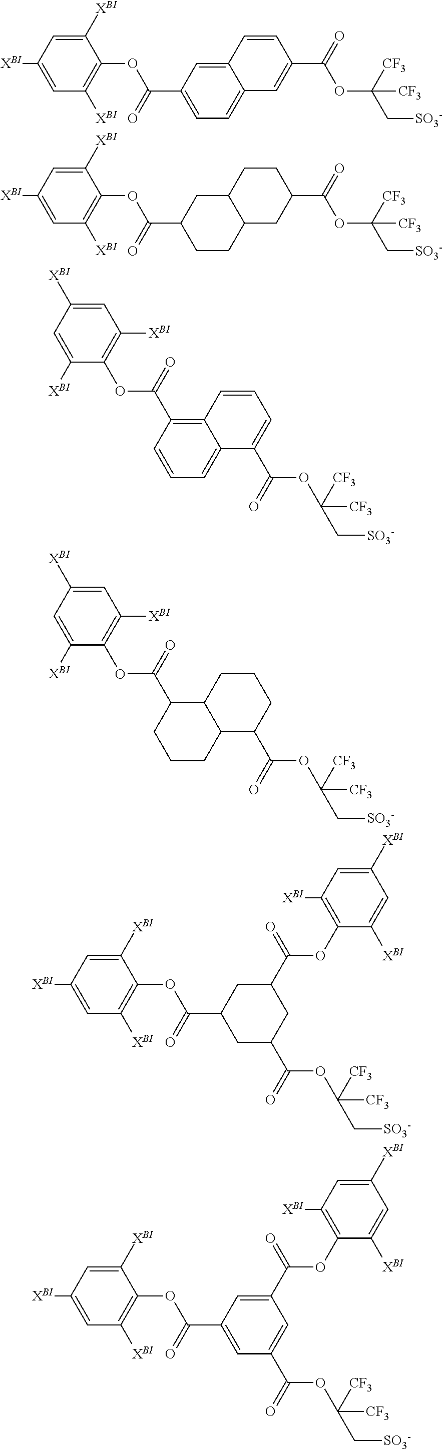

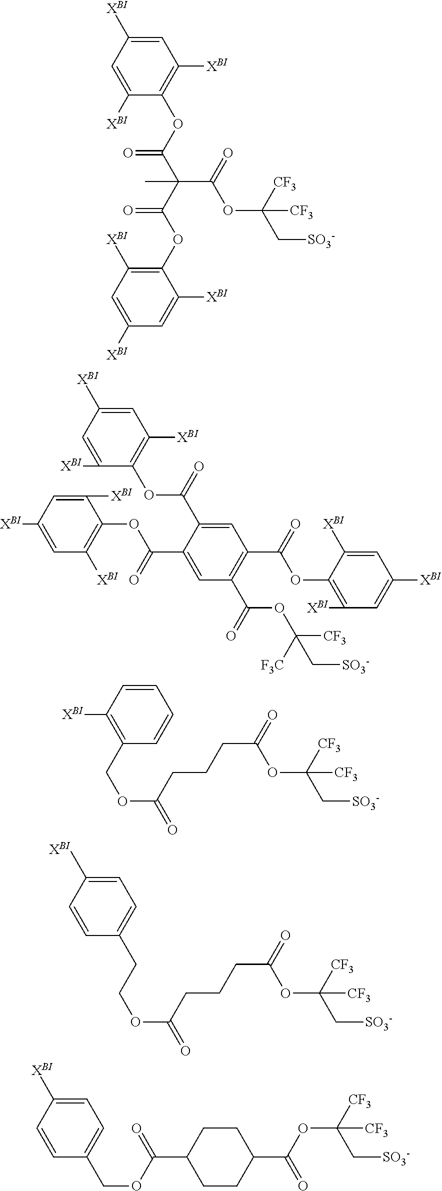

As the non-nucleophilic counter ion, fluorobenzenesulfonate anions having an iodized aromatic group bonded thereto as described in JP 6648726, anions having an acid-catalyzed decomposition mechanism as described in WO 2021/200056 and JP-A 2021-070692, anions having a cyclic ether group as described in JP-A 2018-180525 and JP-A 2021-035935, and anions as described in JP-A 2018-092159 can also be used.

Further, as the non-nucleophilic counter ion, bulky fluorine-free benzenesulfonate anions as described in JP-A 2006-276759, JP-A 2015-117200, JP-A 2016-065016, and JP-A 2019-202974, and fluorine-free benzenesulfonic acid or alkylsulfonate anions having an iodized aromatic group bonded thereto as described in JP 6645464 can also be used.

Furthermore, as the non-nucleophilic counter ion, bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-024989 can also be used.

In the formulae (c2) and (c3), d1 and d2 are each independently 0, 1, 2, or 3, preferably 1.

In the formula (c4), e1 is 0 or 1. e2 is 0, 1, 2, 3, or 4. e3 is 0, 1, 2, 3, or 4.

It is noted that 0≤e2+e3≤4 in case of e1=0 and 0≤e2+e3≤6 in case of e1=1.

In the formulae (c2), (c3), and (C4), L¹ is a single bond, ether bond, ester bond, carbonyl, sulfonate ester bond, sulfonamide bond, carbonate bond or carbamate bond.

From the aspect of synthesis, an ether bond, ester bond or carbonyl group is preferred, with the ester bond or carbonyl being more preferred.

In the formula (c2), Rf¹ and Rf² are each independently fluorine or a C₁-C₆ fluorinated saturated hydrocarbyl group. It is preferred that both Rf¹ and Rf² be fluorine because the generated acid has a higher acid strength. Rf³ and Rf⁴ are each independently hydrogen, fluorine, or a C₁-C₆ fluorinated saturated hydrocarbyl group. It is preferred for solvent solubility that at least one of Rf³ and Rf⁴ be trifluoromethyl.

In the formula (c3), Rf⁵ and Rf⁶ are each independently hydrogen, fluorine, or a C₁-C₆ fluorinated saturated hydrocarbyl group. It is excluded that all Rf⁵ and Rf⁶ are hydrogen at the same time. It is preferred for solvent solubility that at least one of Rf⁵ and Rf⁶ be trifluoromethyl.

In the formula (c4), Rf⁷ is fluorine, a C₁-C₆ fluorinated alkyl group, a C₁-C₆ fluorinated alkoxy group, or a C₁-C₆ fluorinated alkylthio group. Rf⁷ is preferably fluorine, a trifluoromethyl, difluoromethyl, trifluoromethoxy, difluoromethoxy, trifluoromethylthio or difluoromethylthio group, more preferably fluorine, a trifluoromethoxy or trifluoromethylthio group. A plurality of Rf⁷ may be identical or different when e2 is 2, 3, or 4.

In the formula (c4), R³³ is halogen exclusive of fluorine, or a C₁-C₂₀ hydrocarbyl group which may contain a heteroatom. The hydrocarbyl group may be saturated or unsaturated and straight, branched or cyclic. Examples thereof include those exemplified above for the hydrocarbyl group represented by R¹ in the formula (1), but not limited thereto. When e3 is 2, 3, or 4, a plurality of R³³ may be identical or different.

Also, e3 is 2, 3, or 4, a plurality of R³³ may bond together to form a ring with the carbon atoms to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

Examples of the anion in repeat unit (c2) include those shown below, but not limited thereto. In the formulae, R^{A} is as defined above, and Me is methyl.

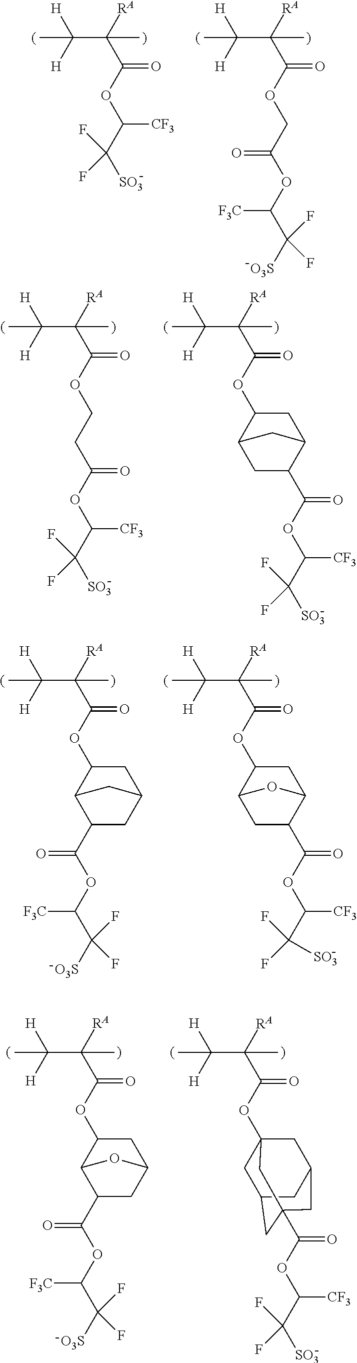

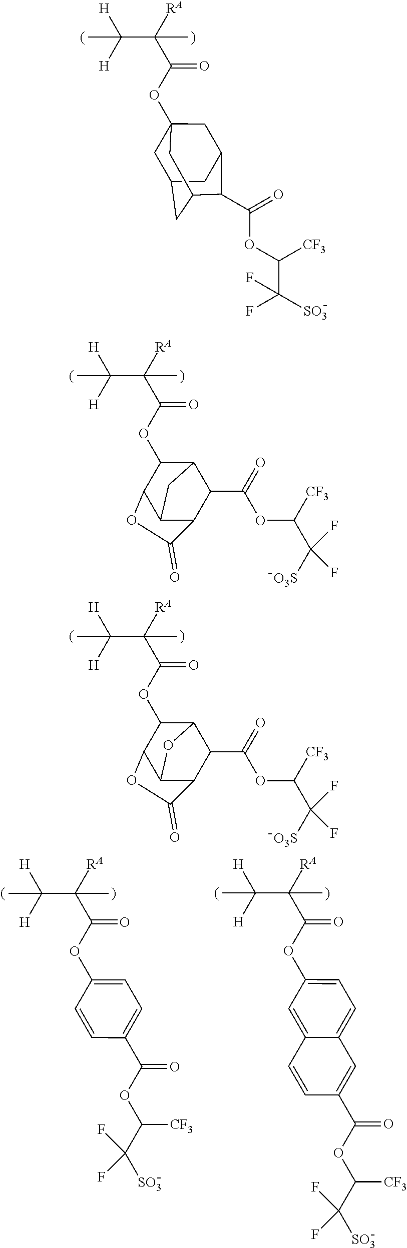

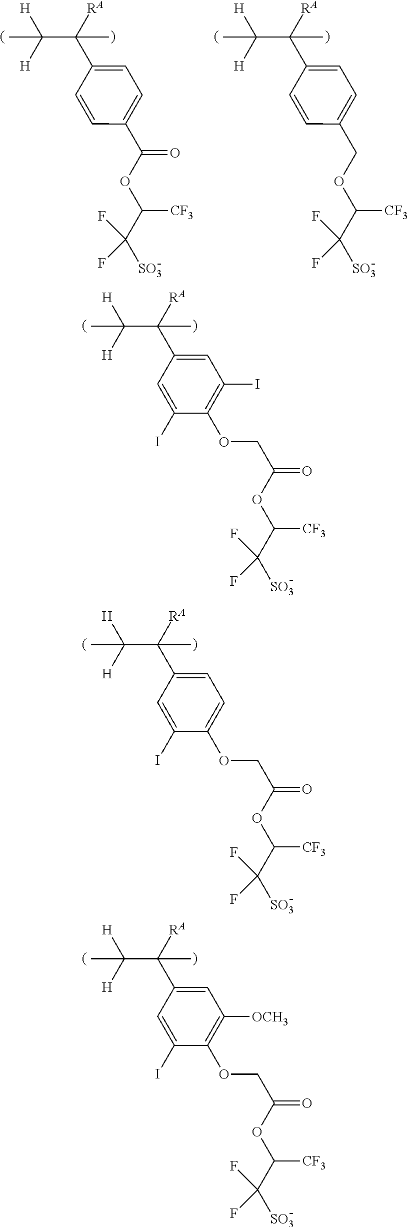

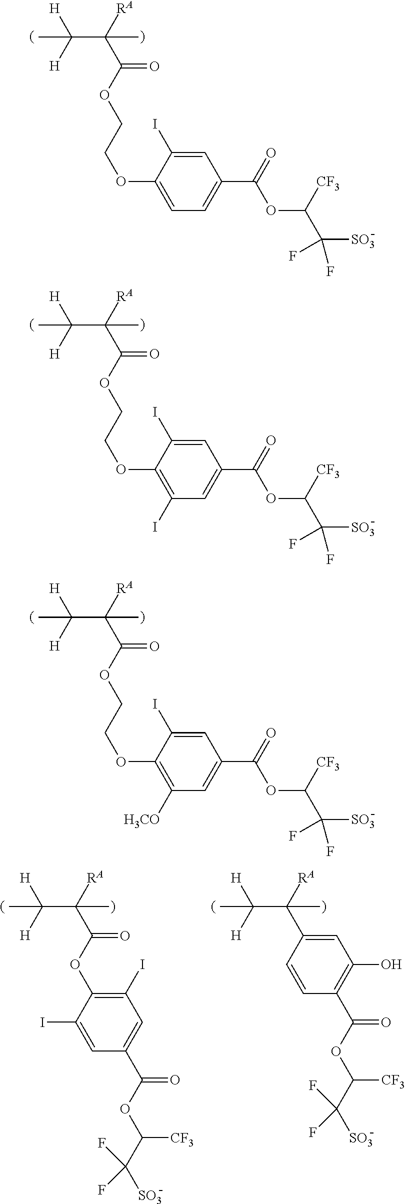

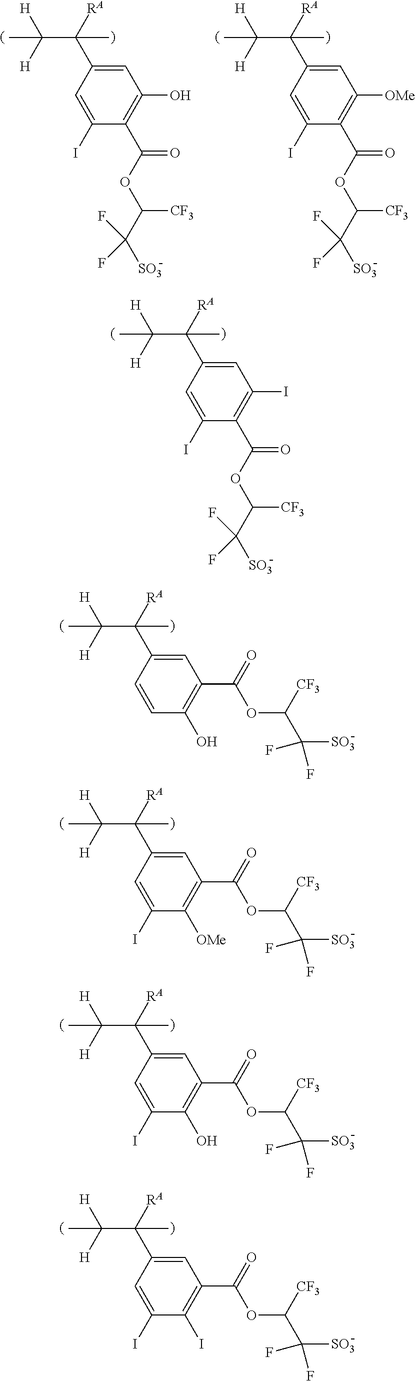

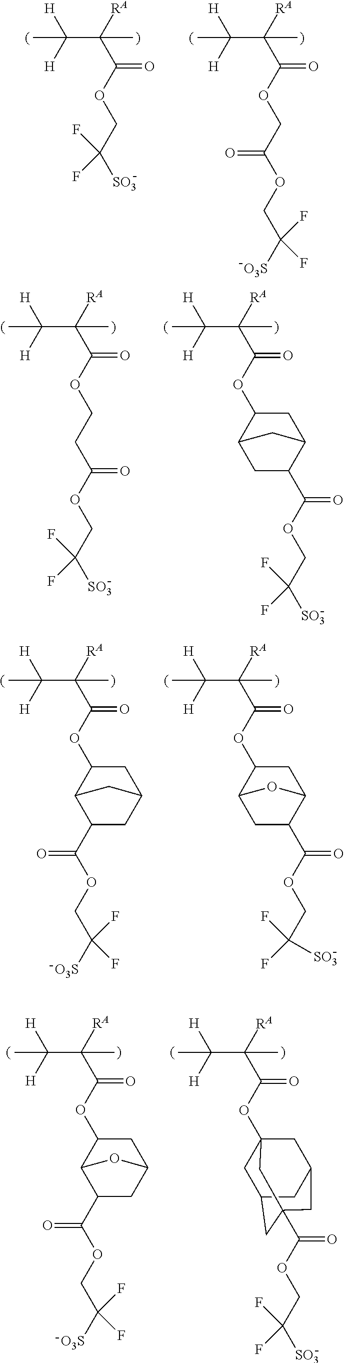

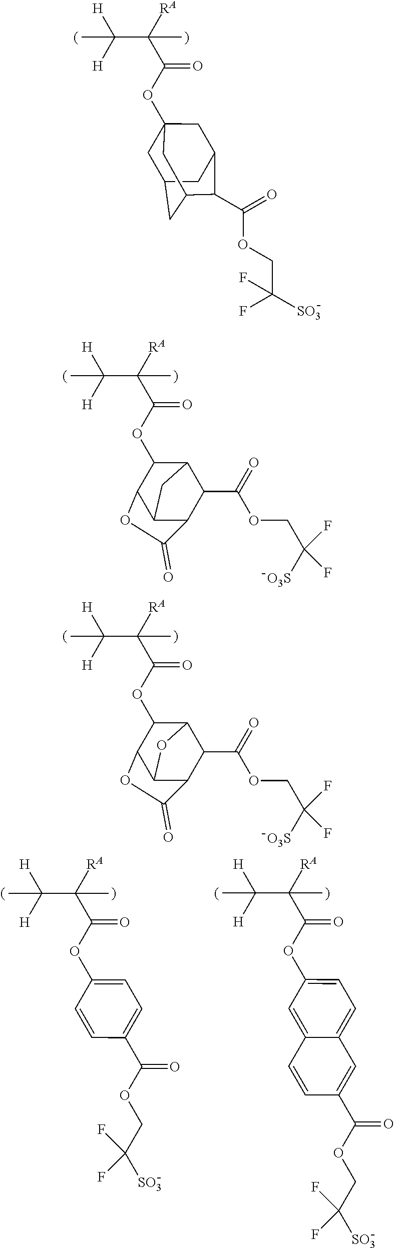

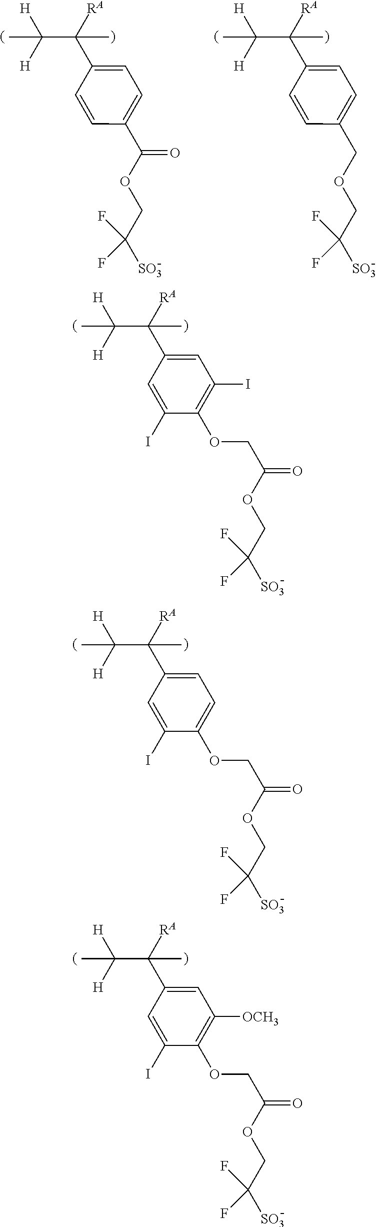

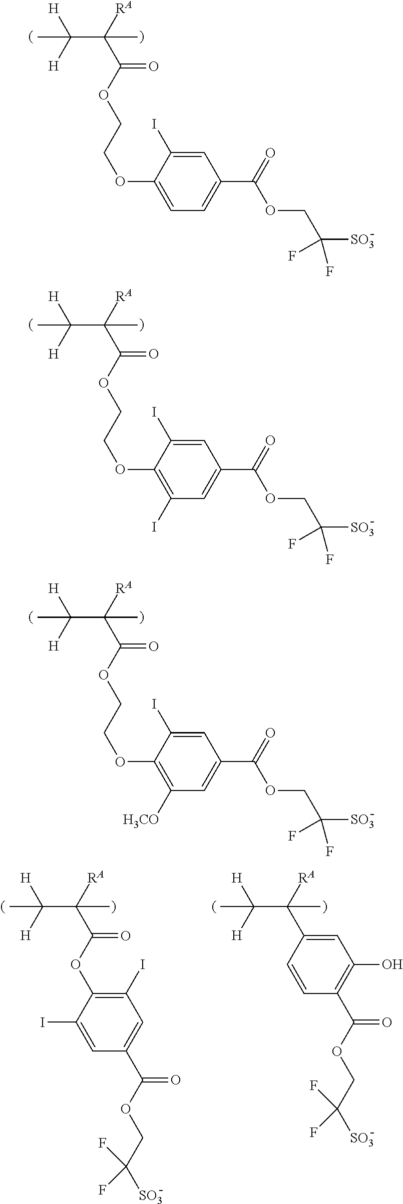

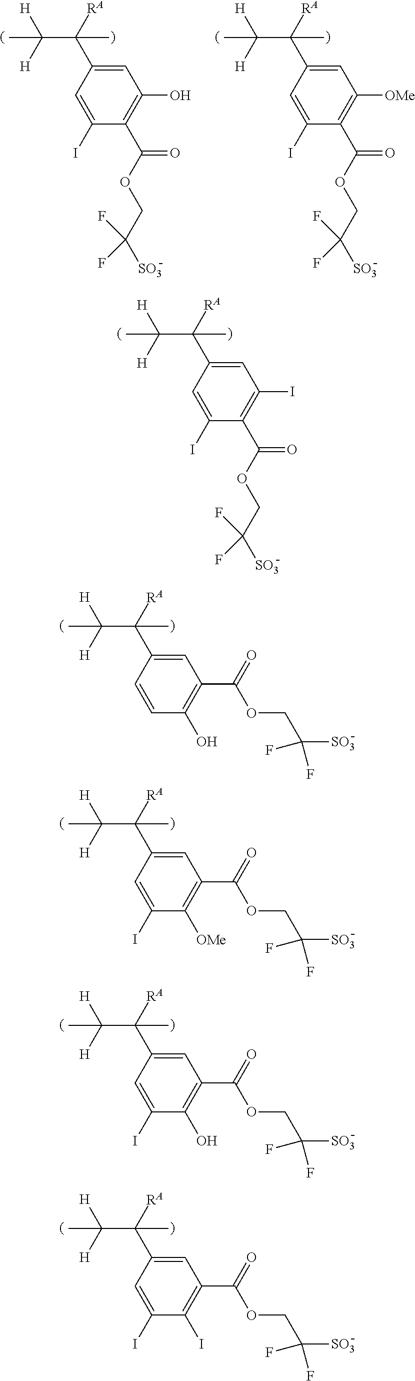

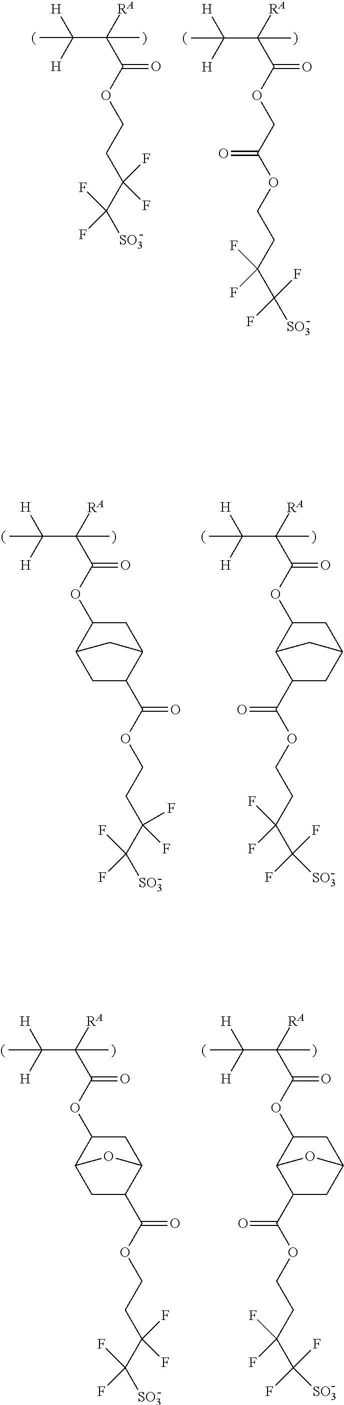

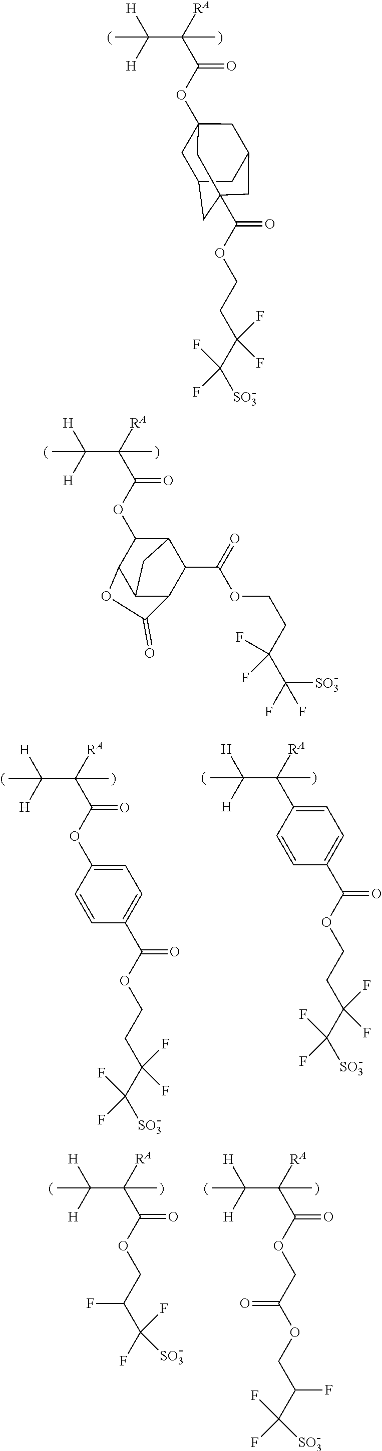

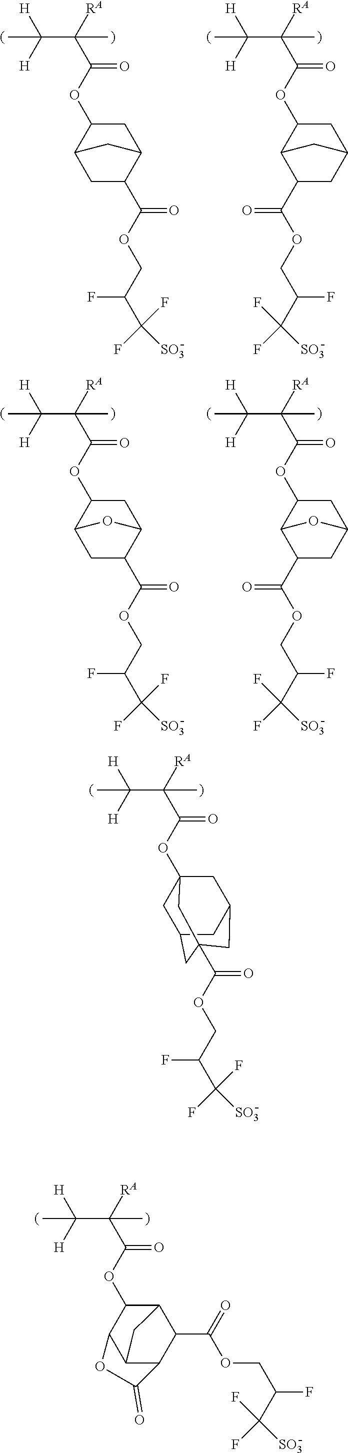

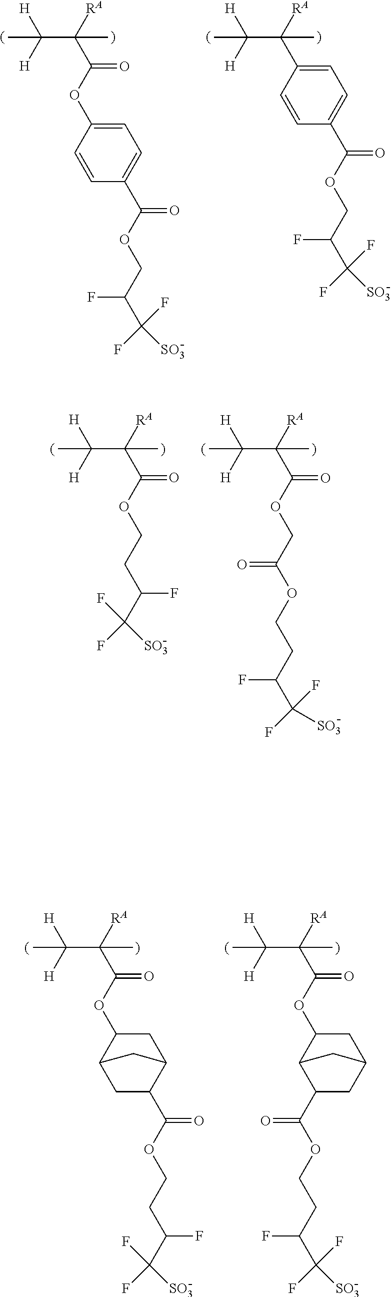

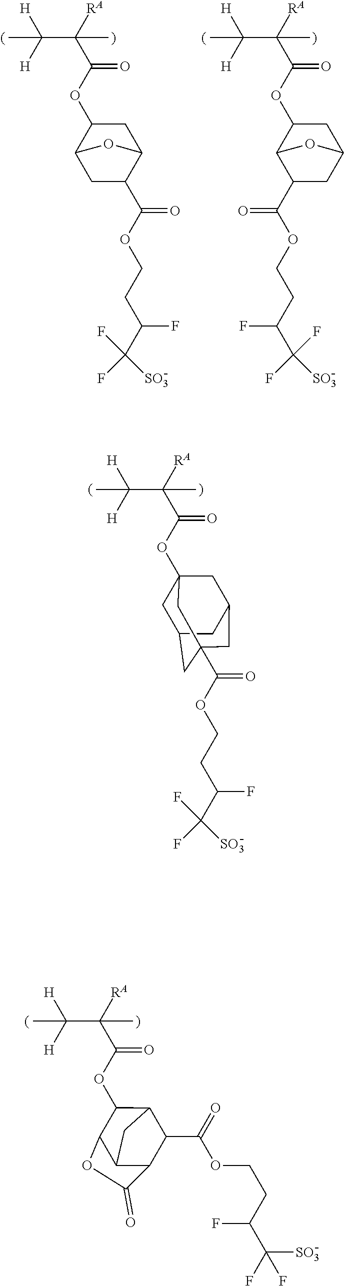

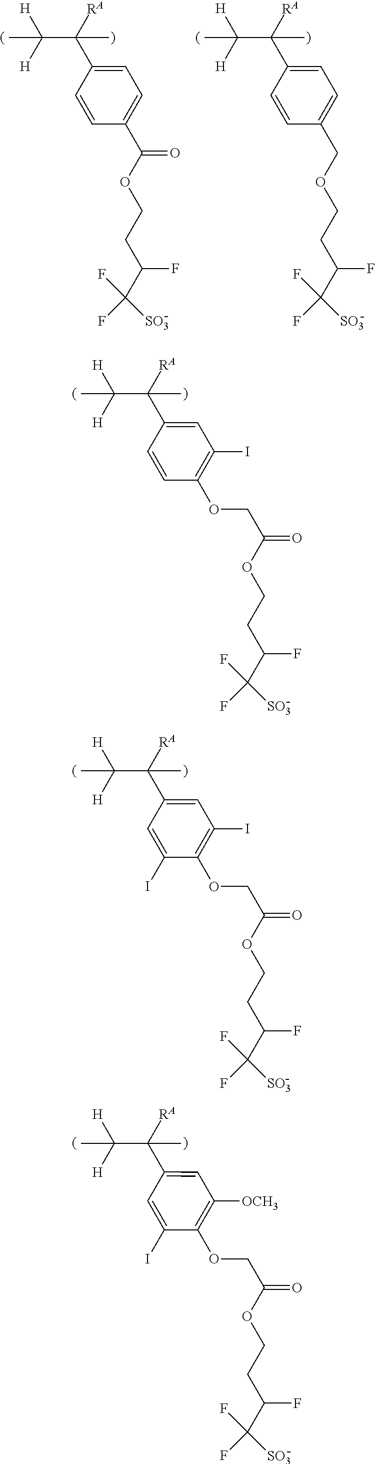

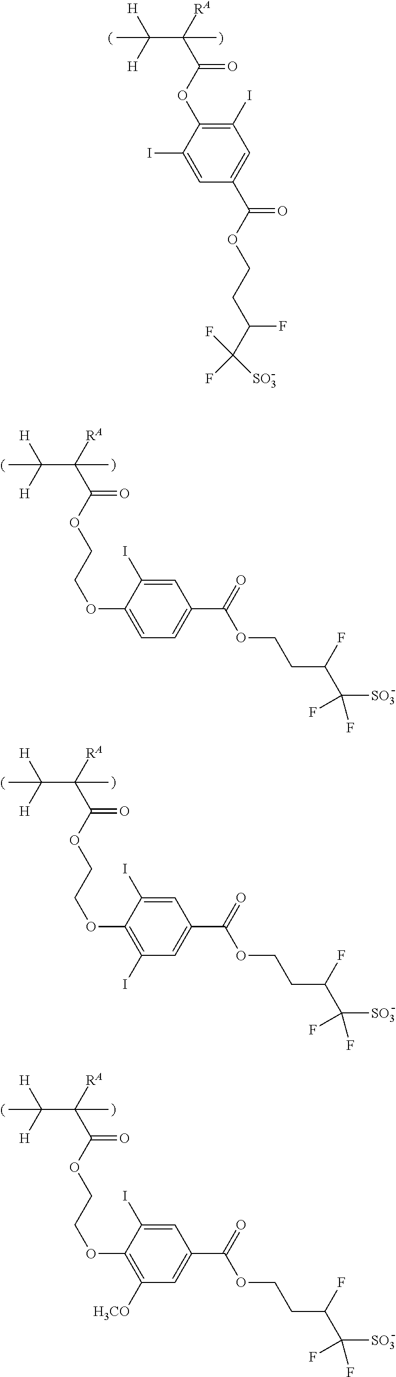

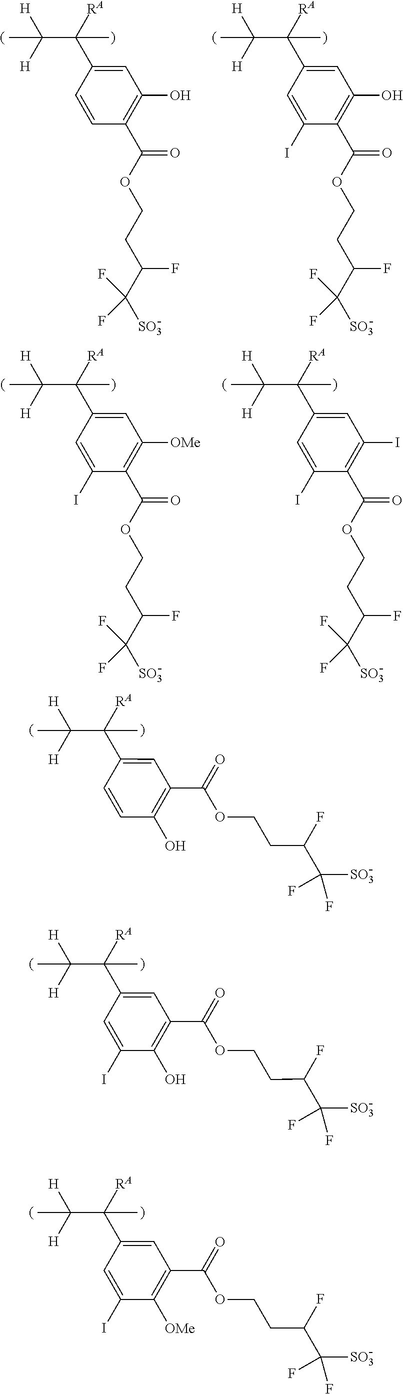

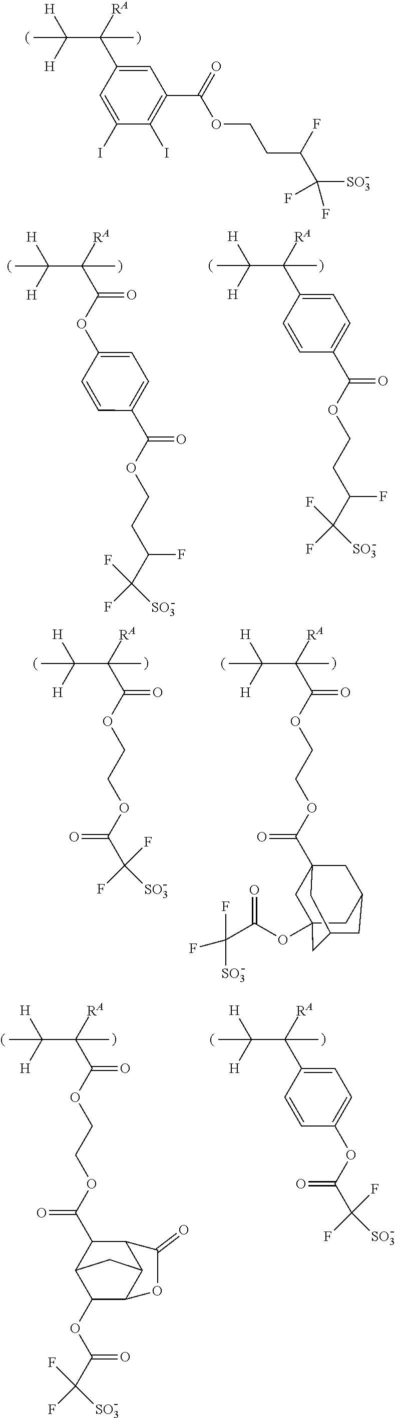

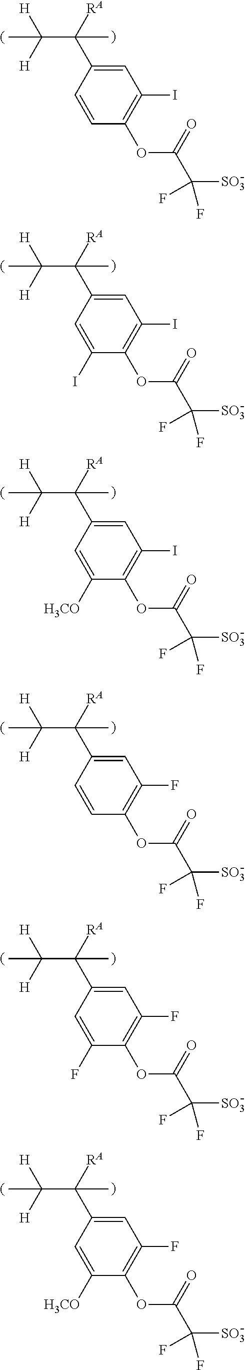

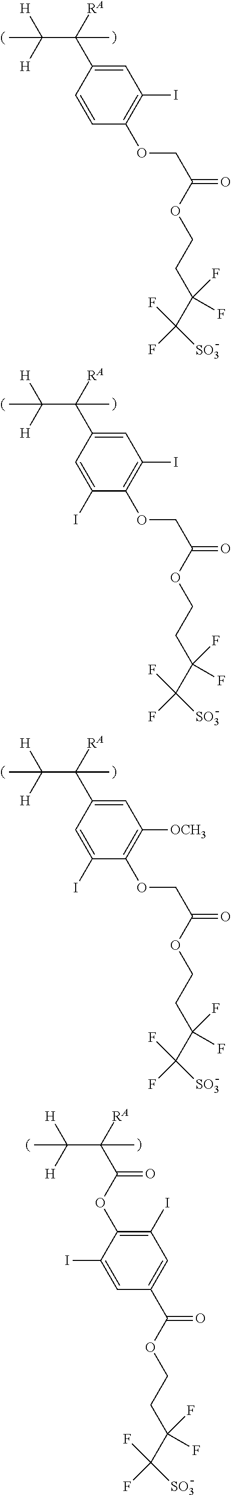

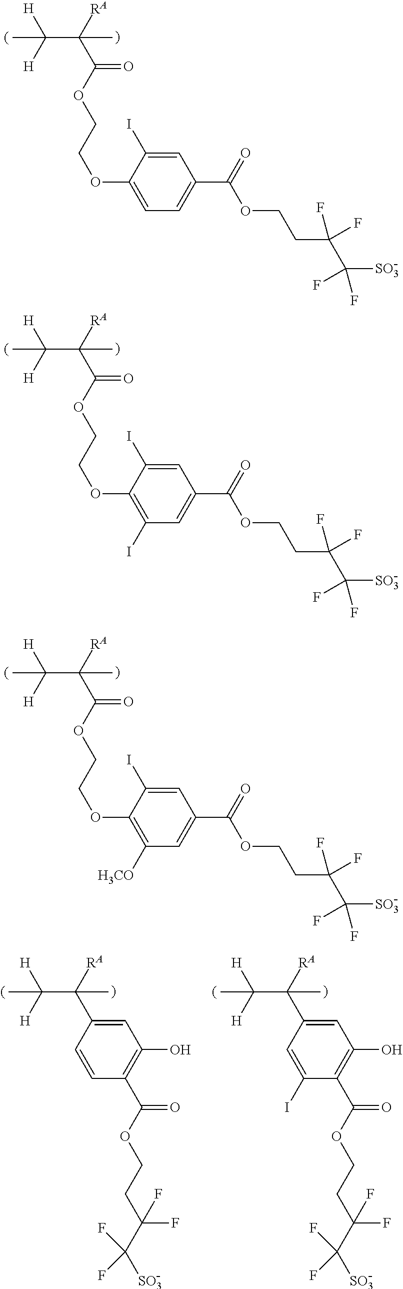

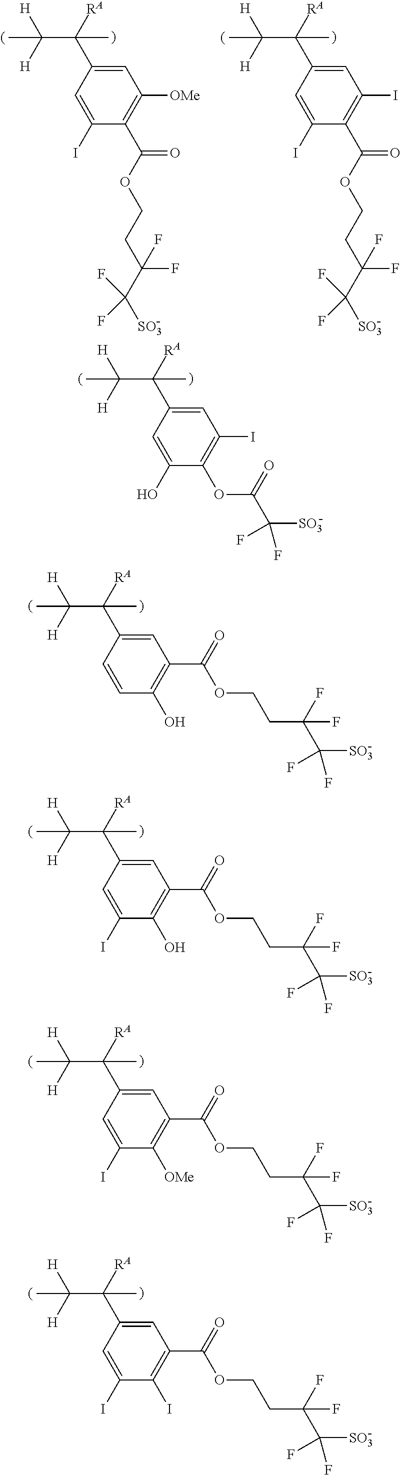

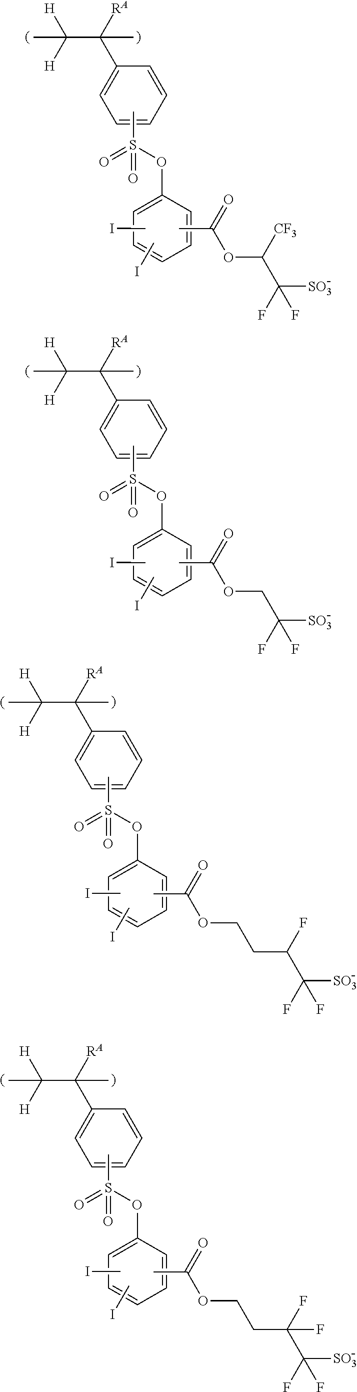

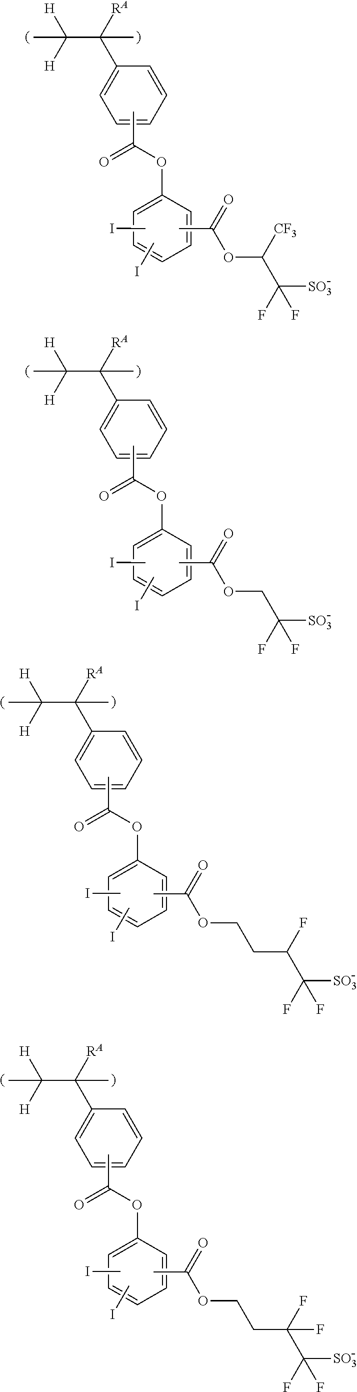

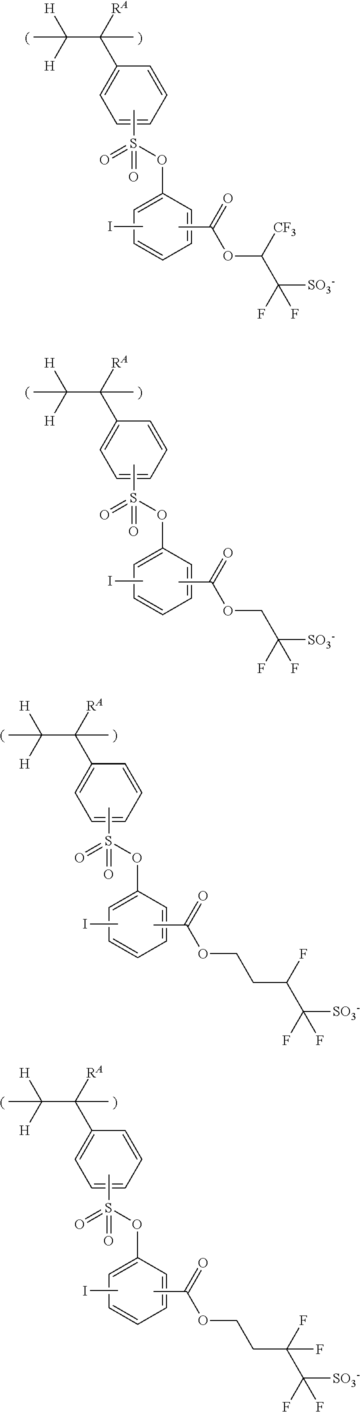

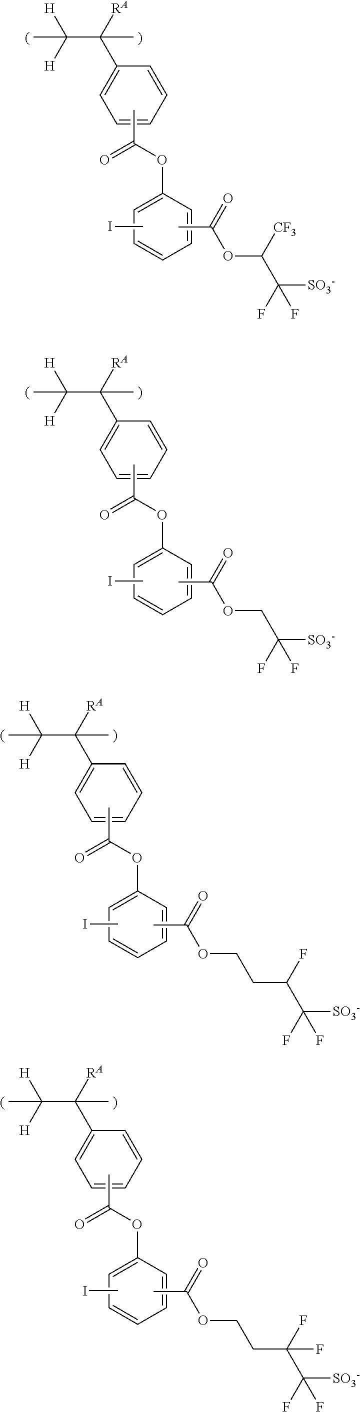

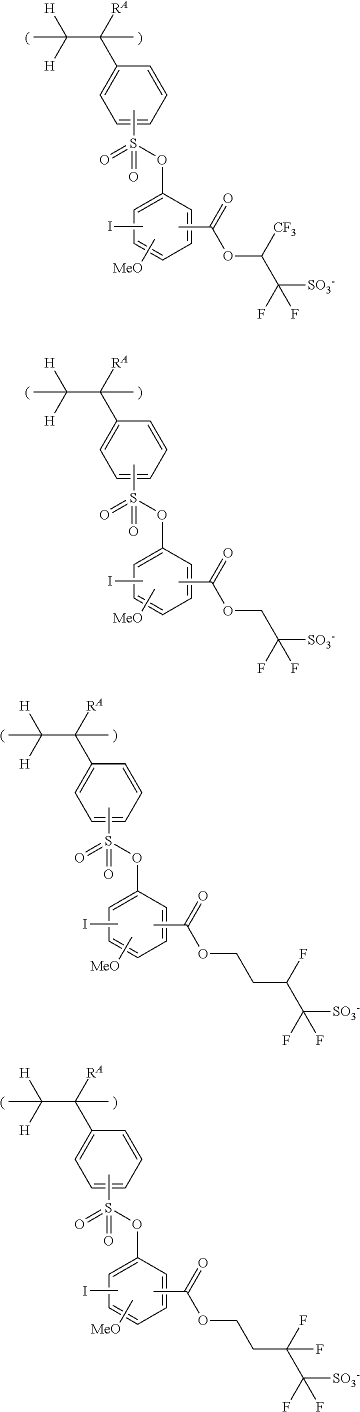

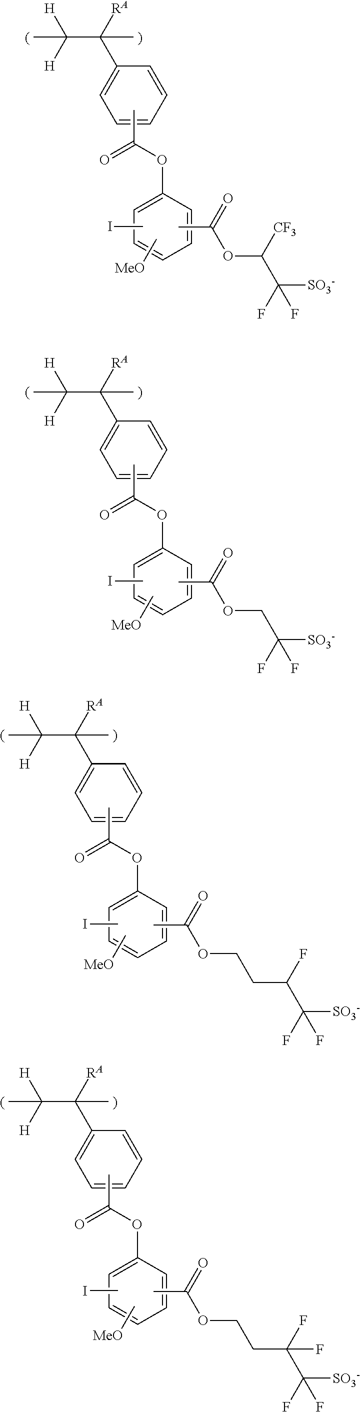

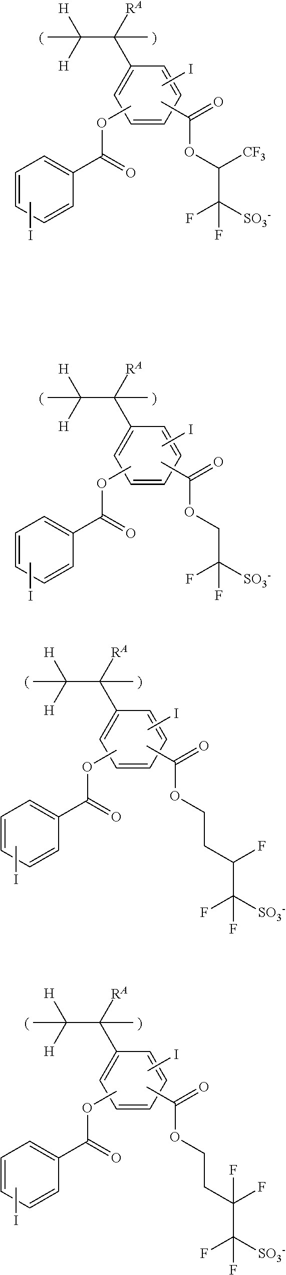

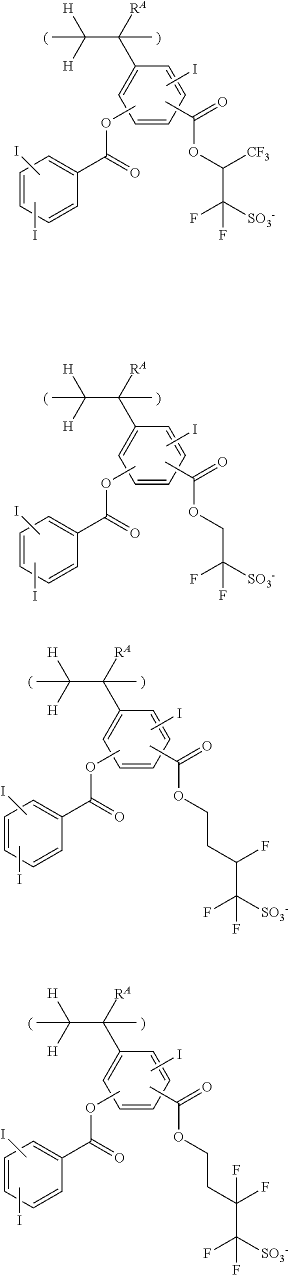

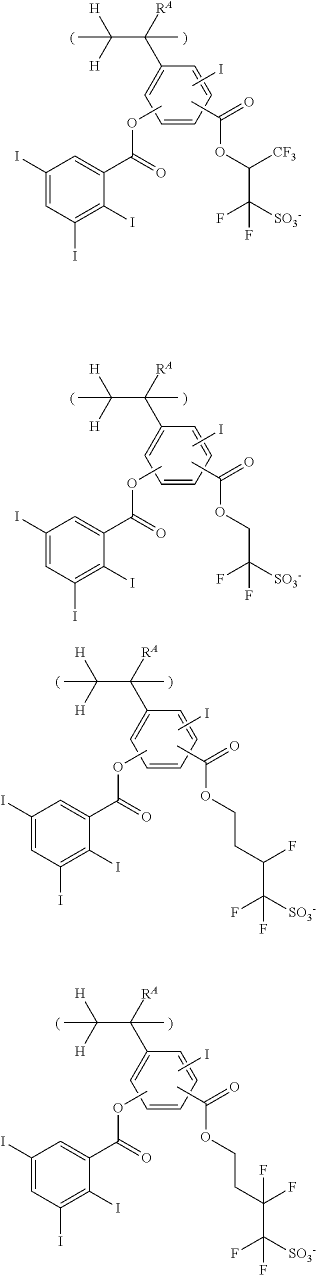

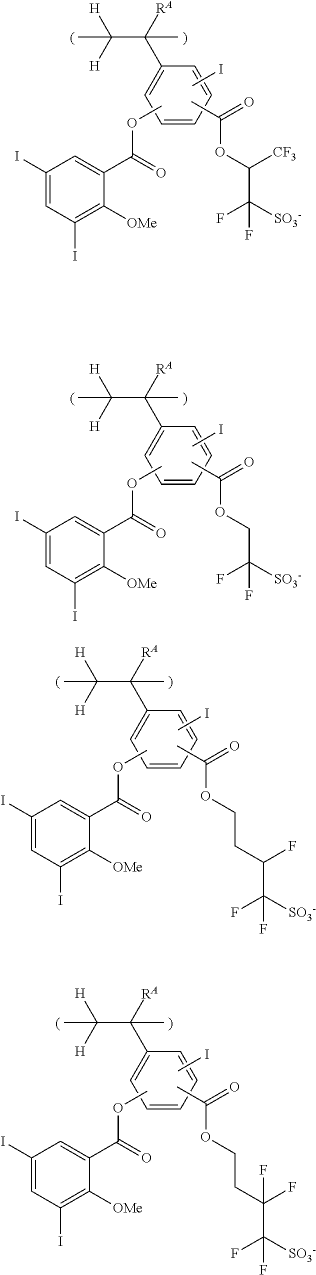

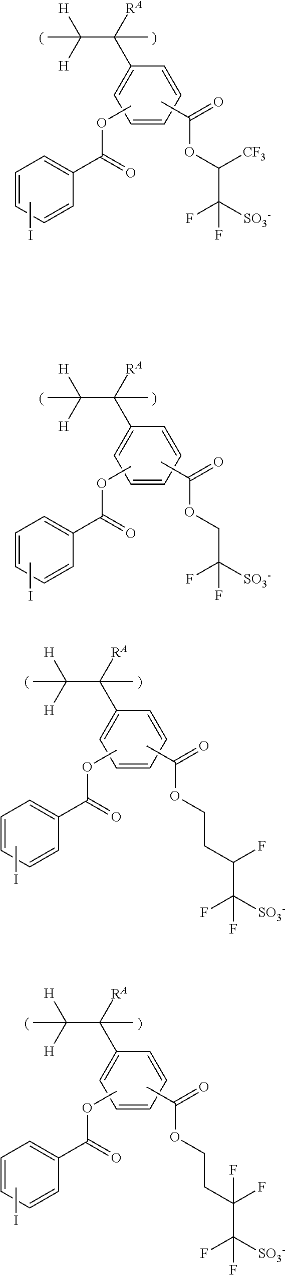

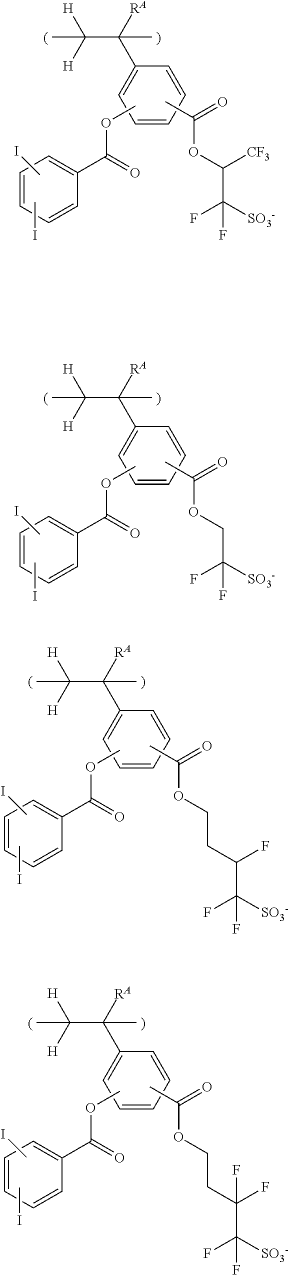

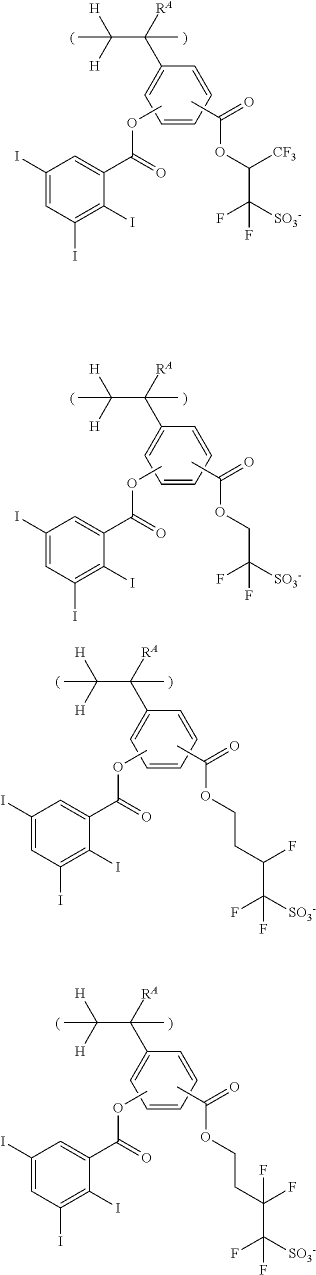

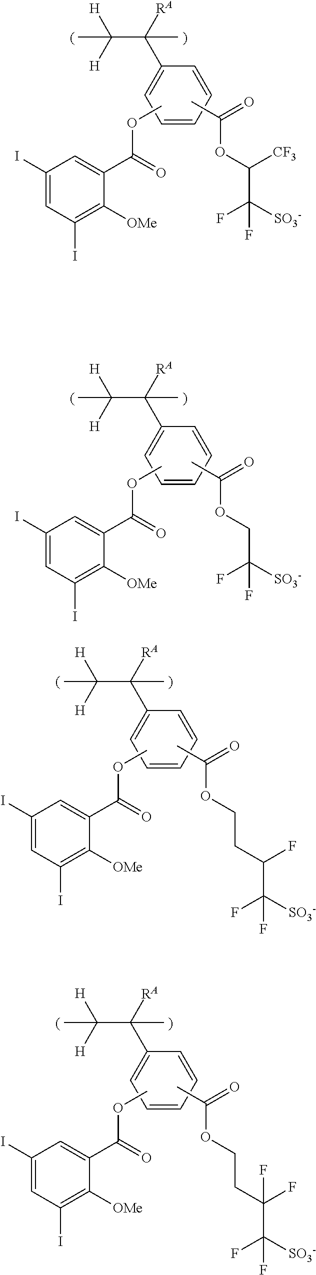

Examples of the anion in repeat unit (6) include those shown below, but not limited thereto. In the formulae, R^{A} is as defined above.

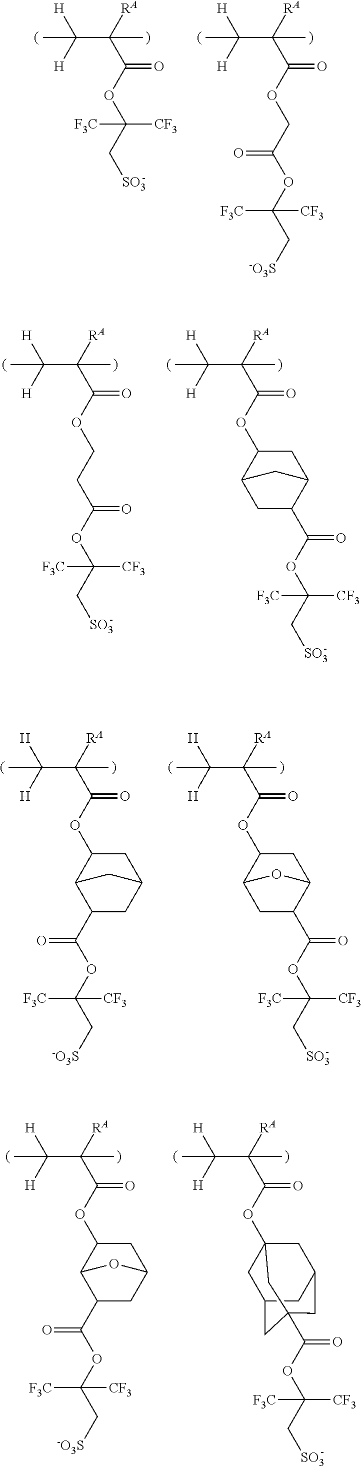

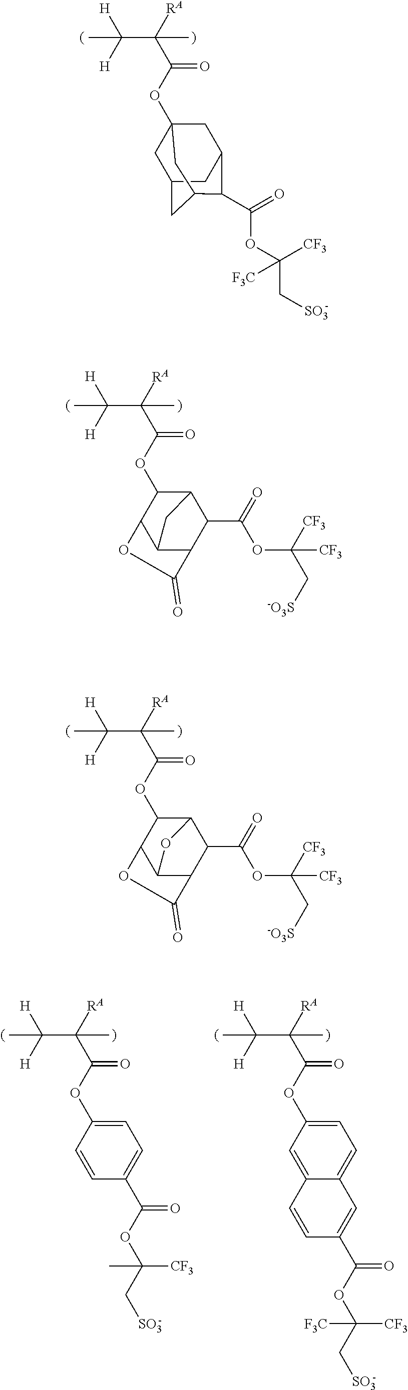

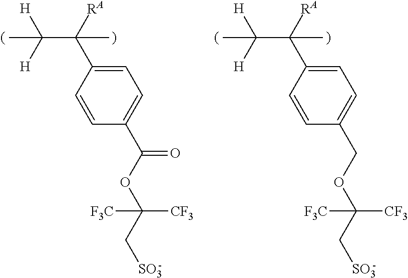

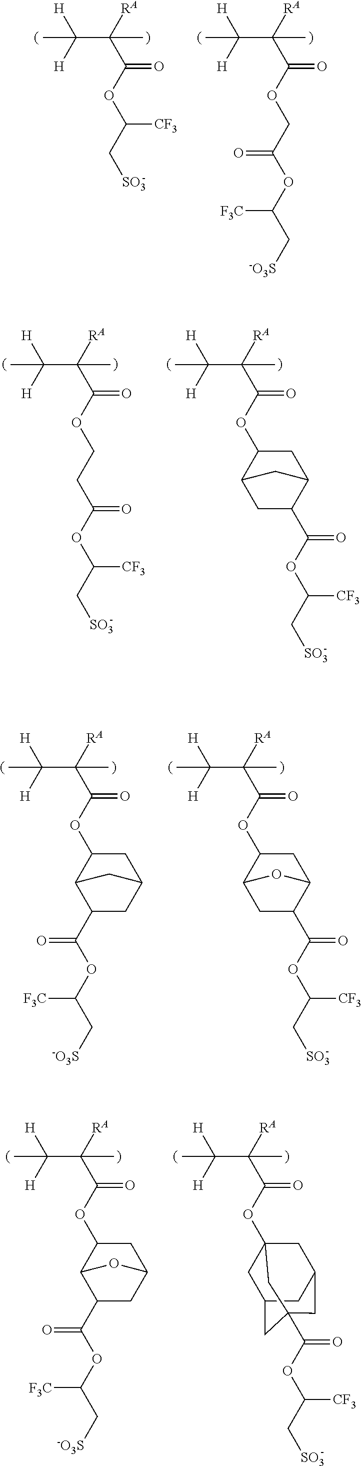

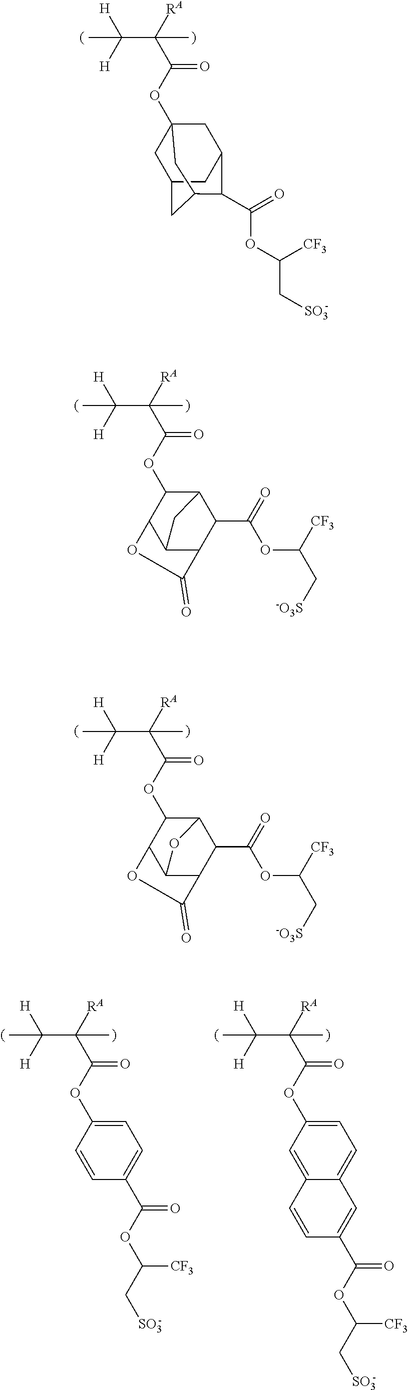

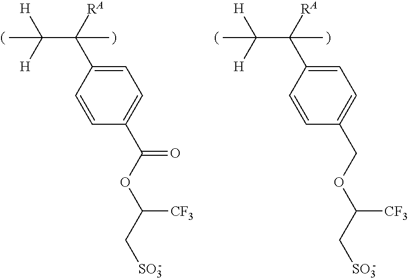

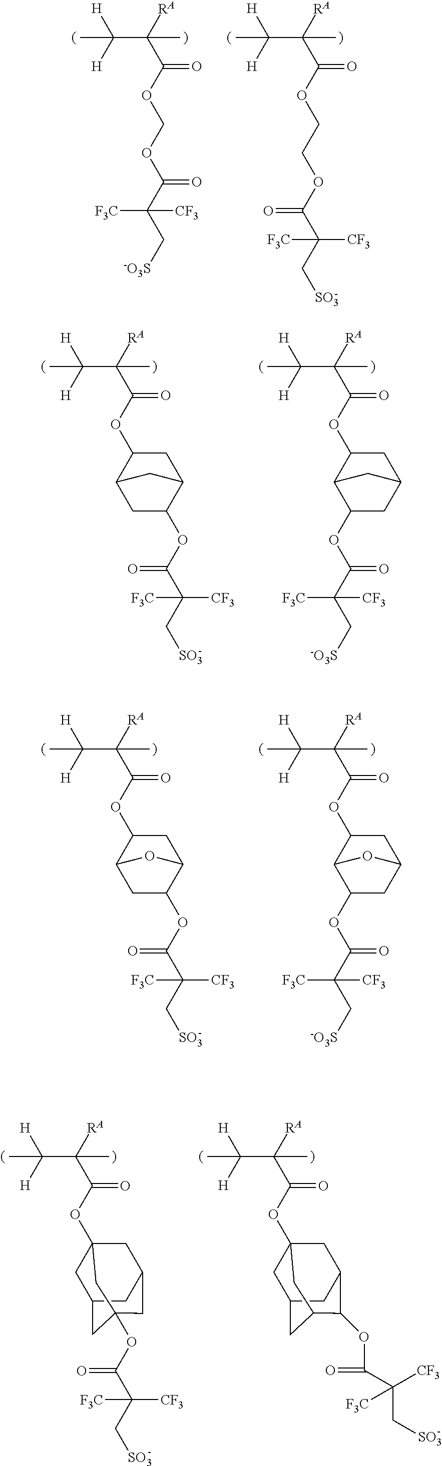

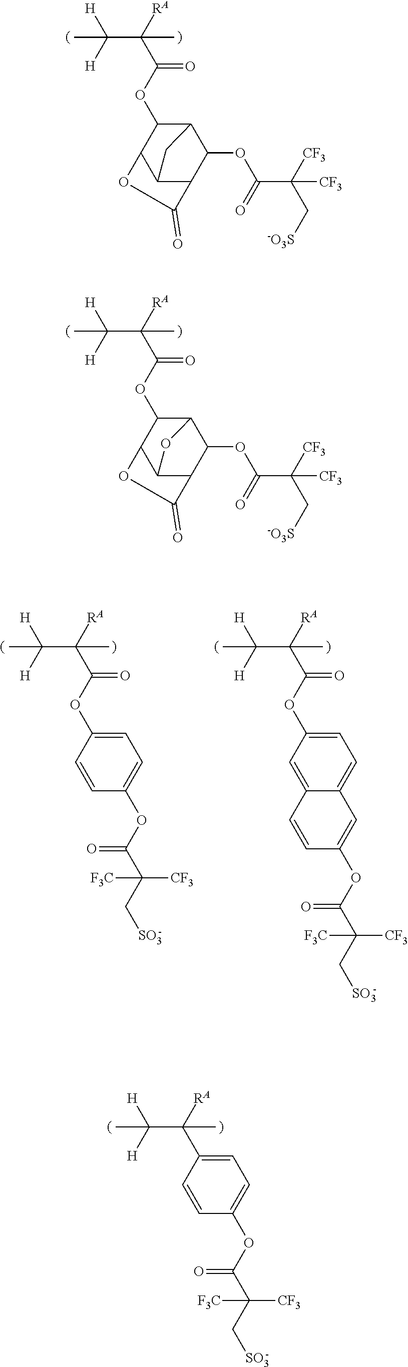

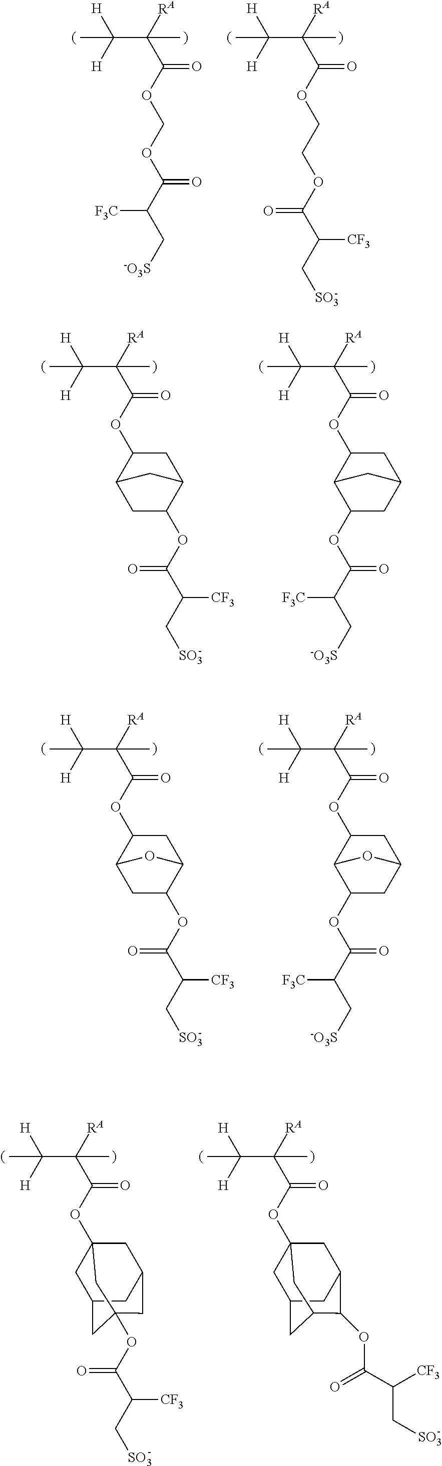

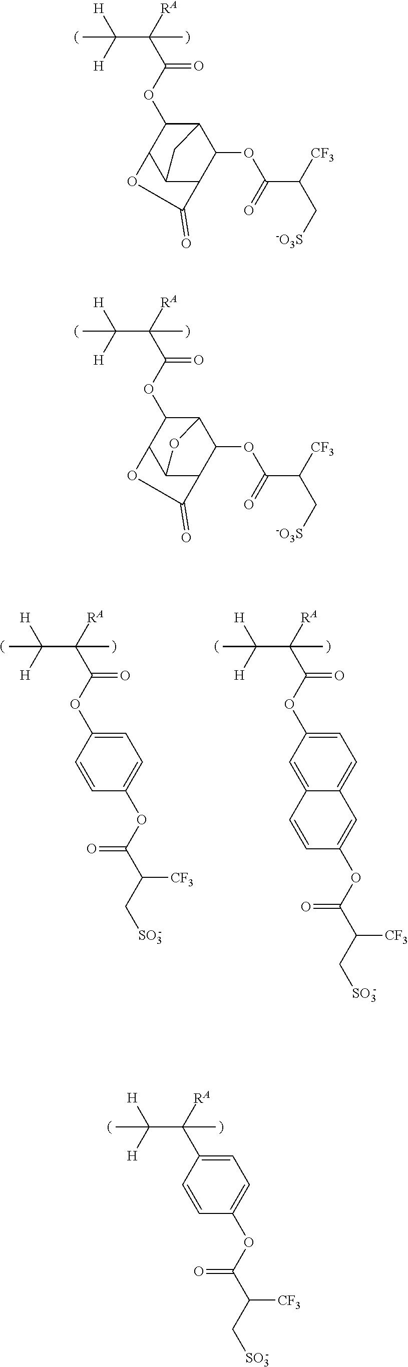

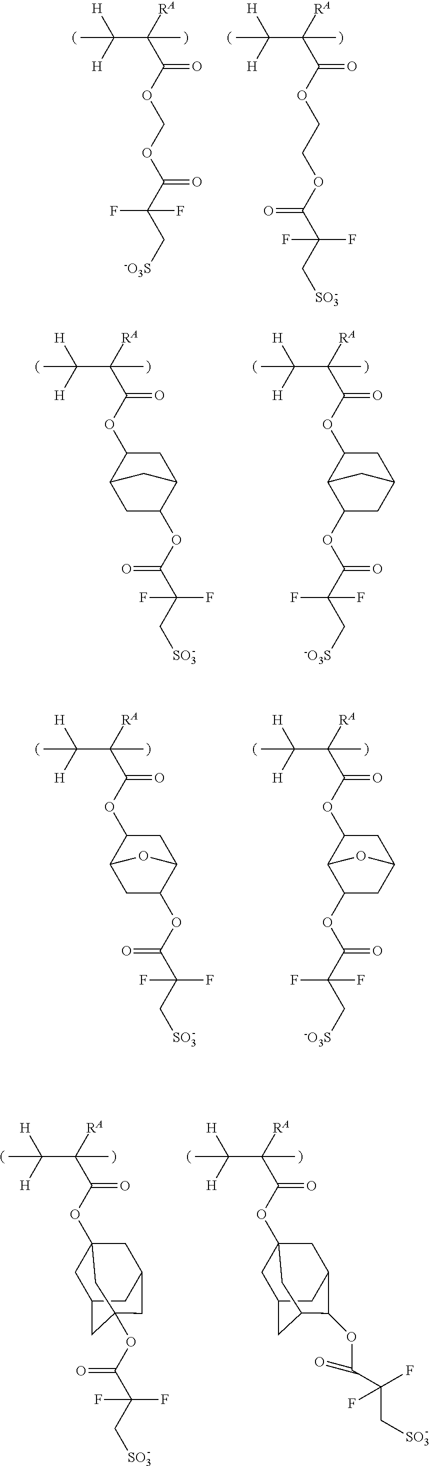

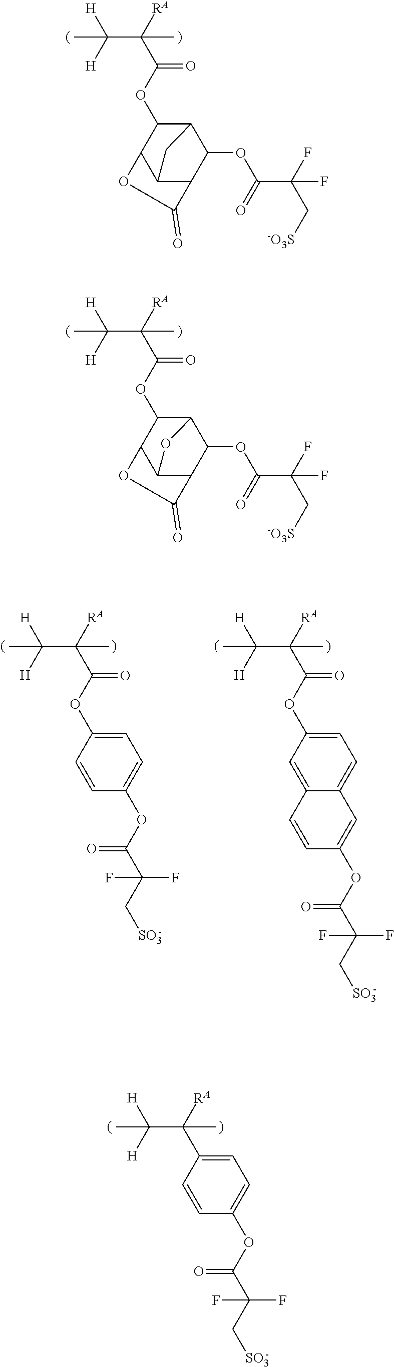

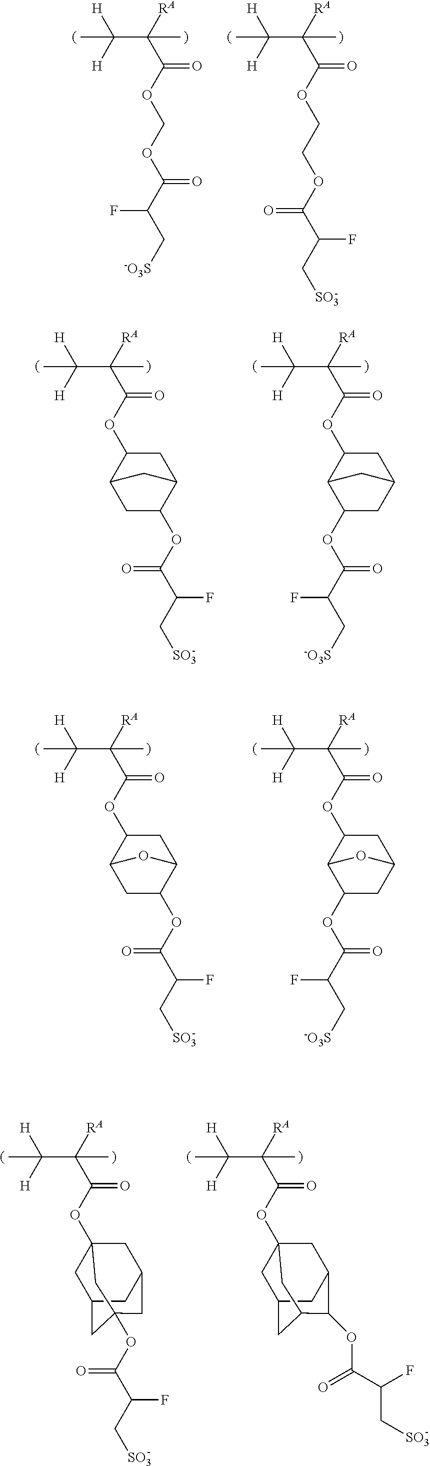

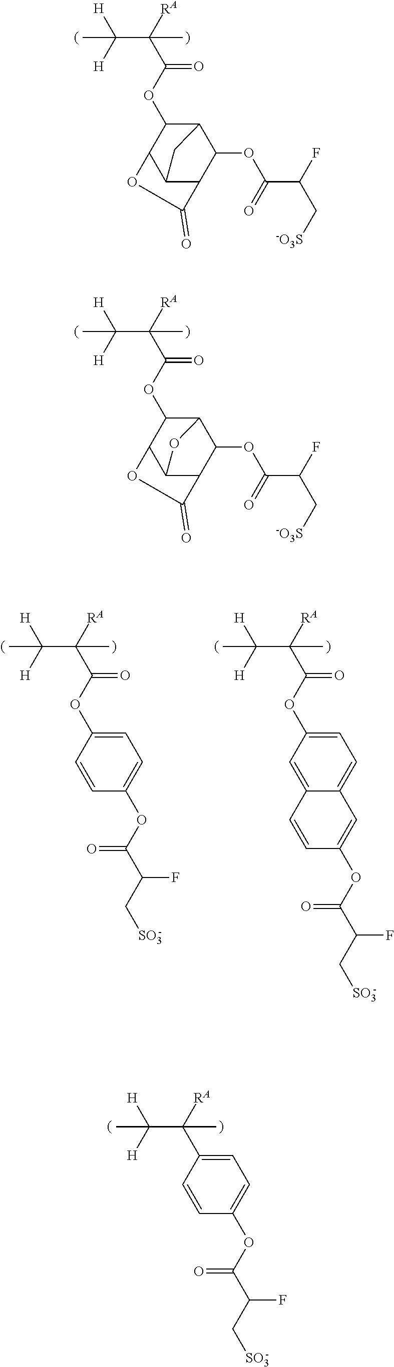

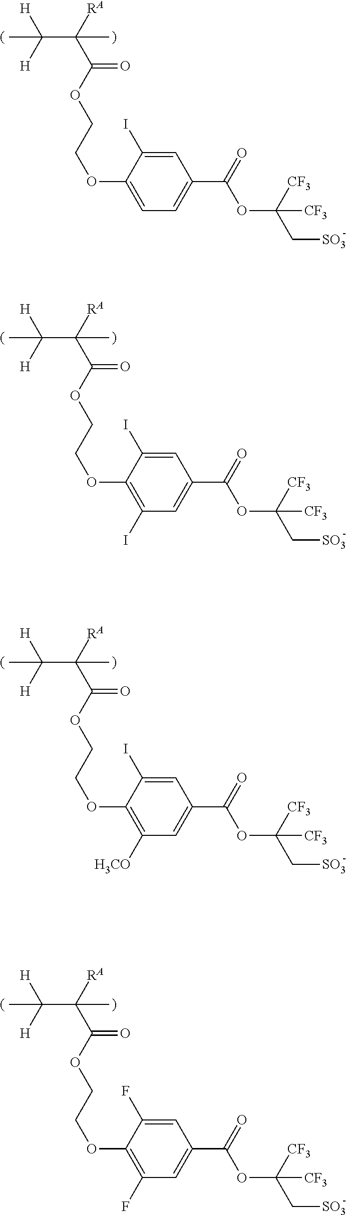

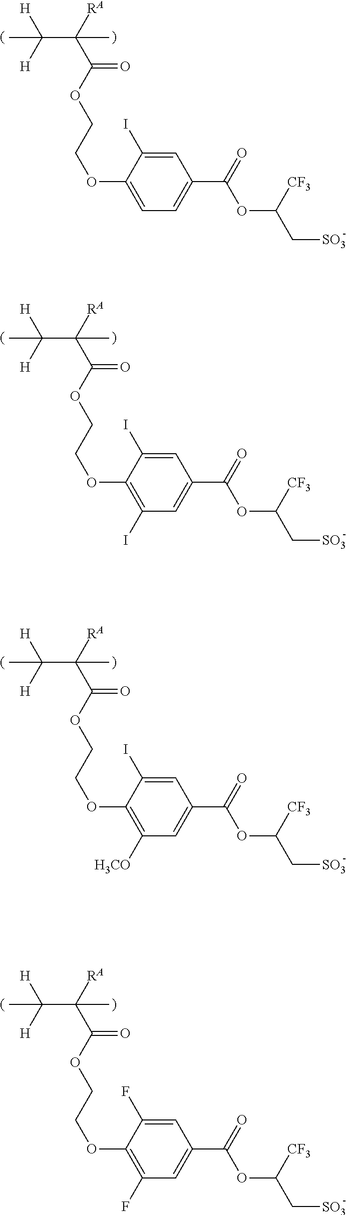

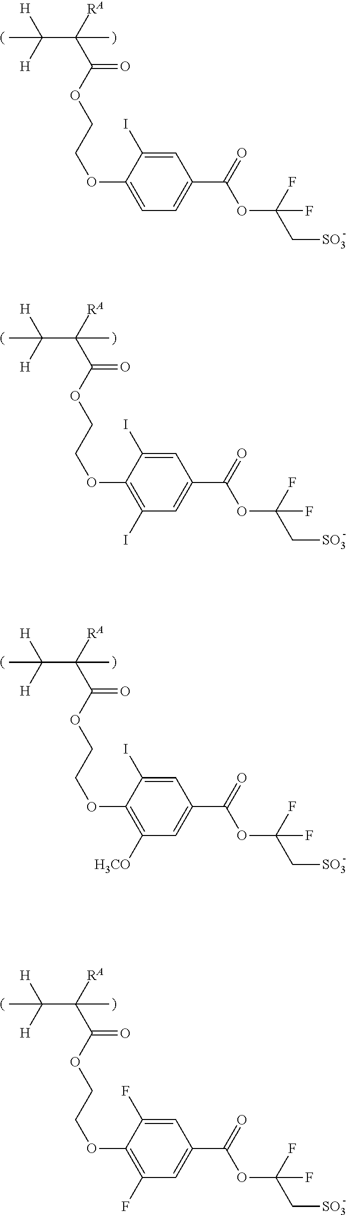

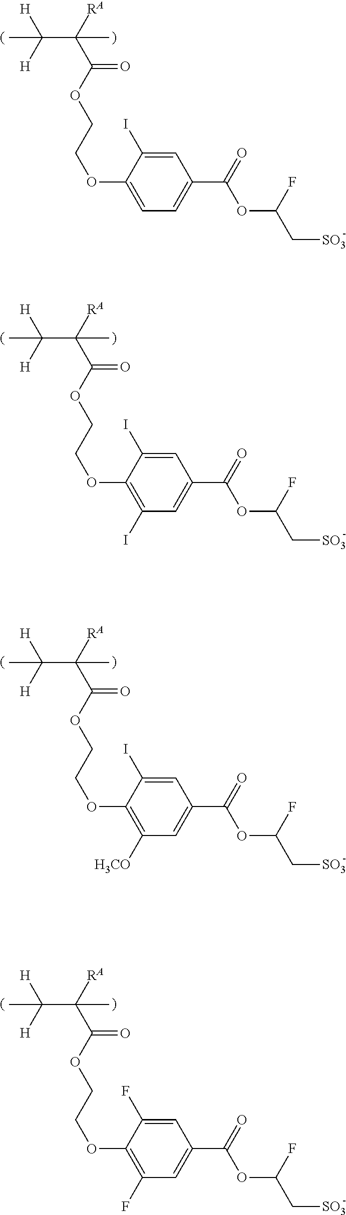

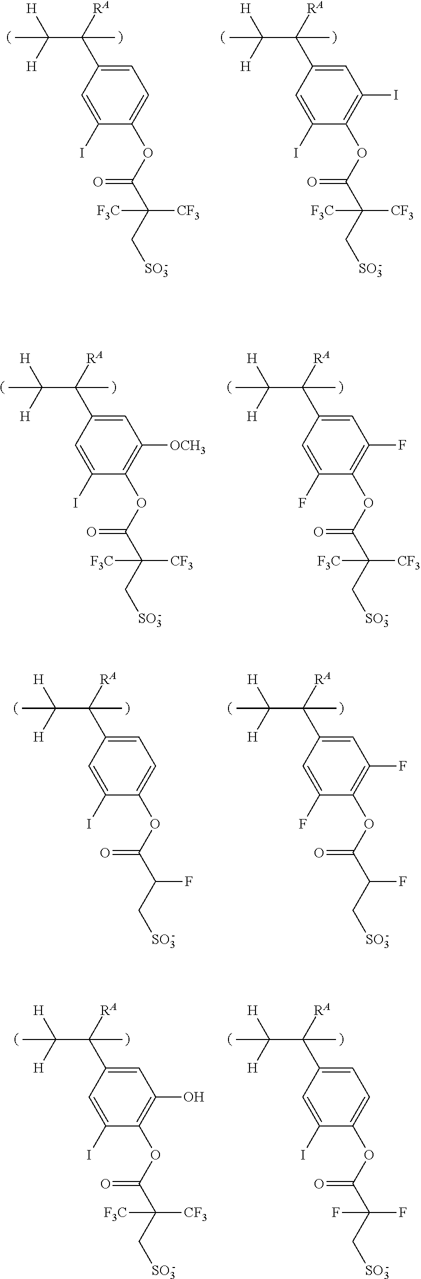

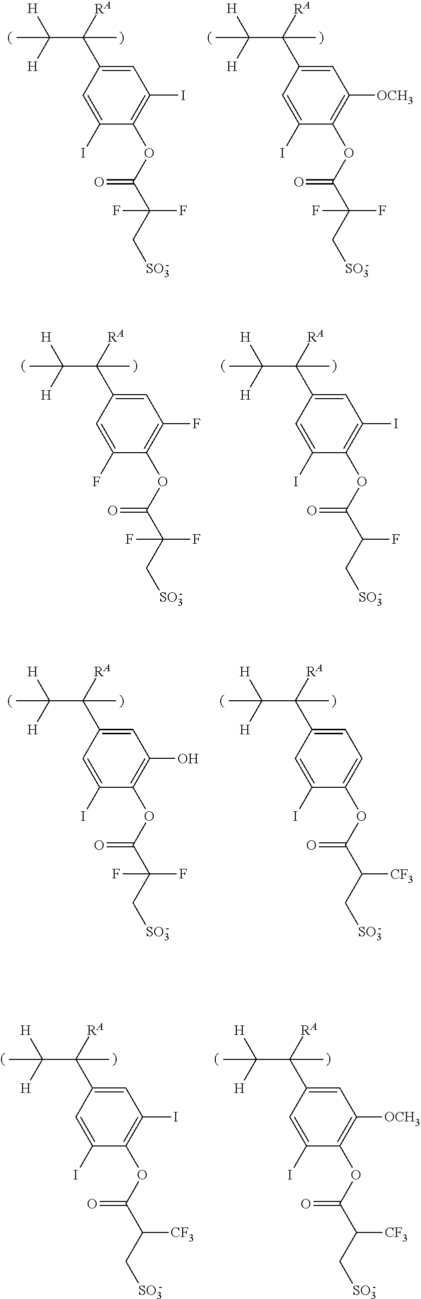

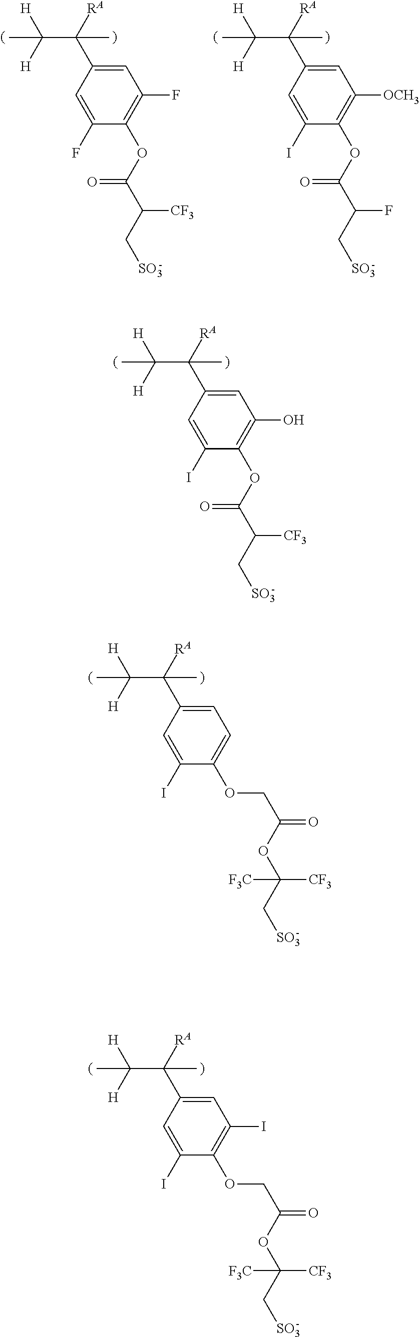

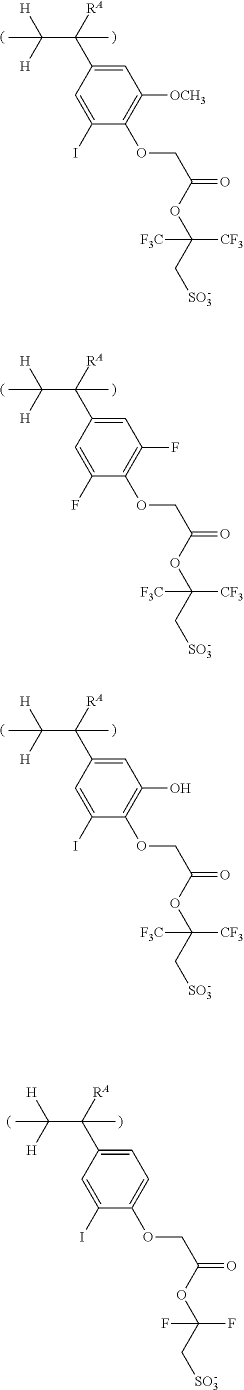

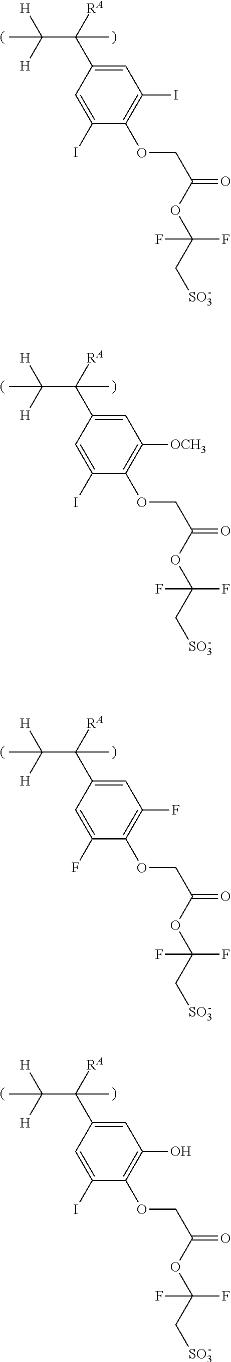

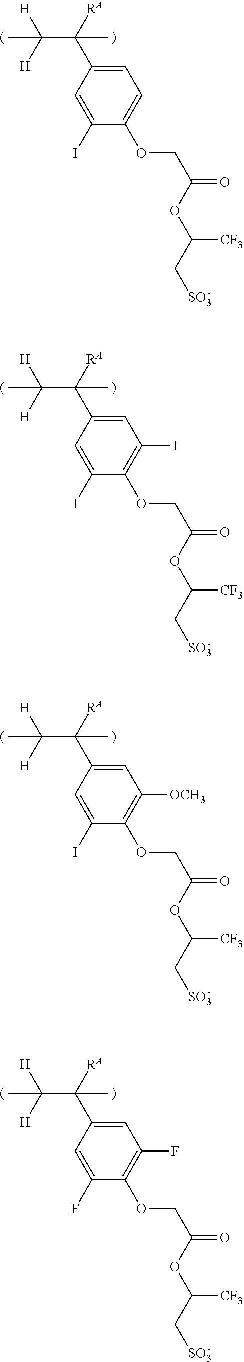

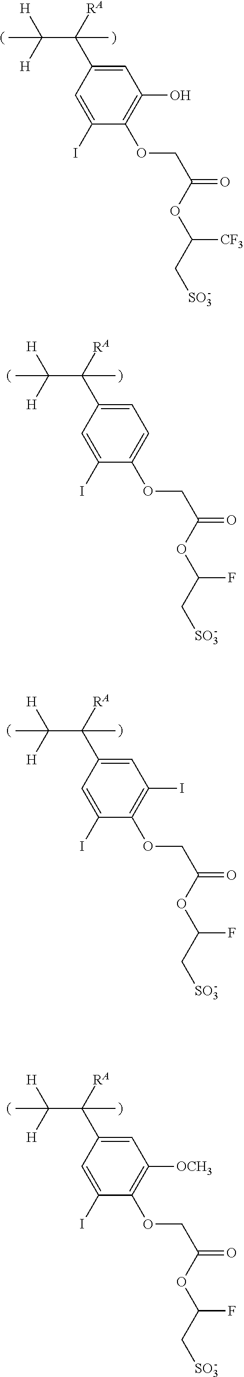

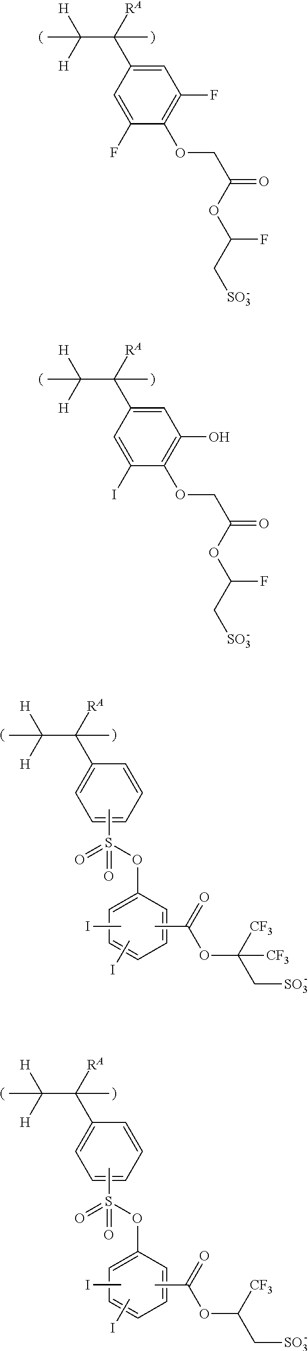

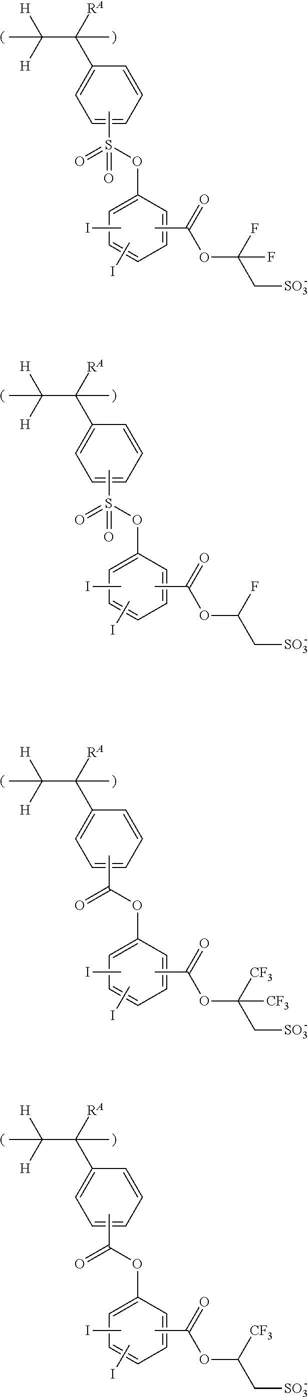

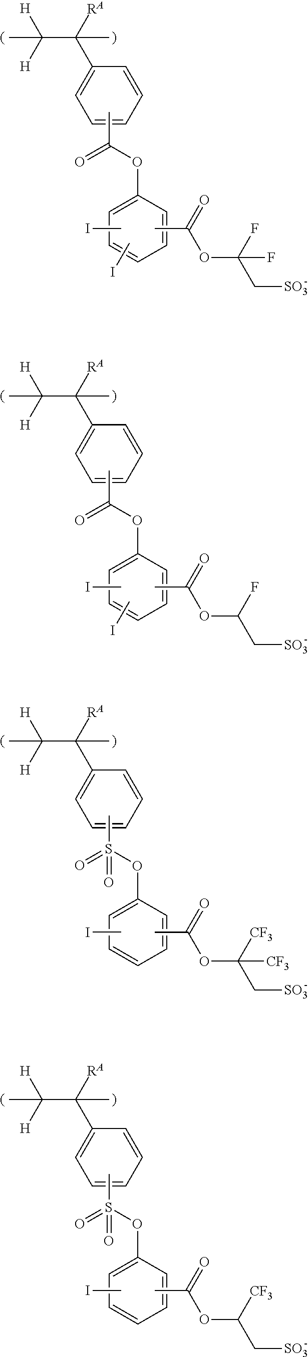

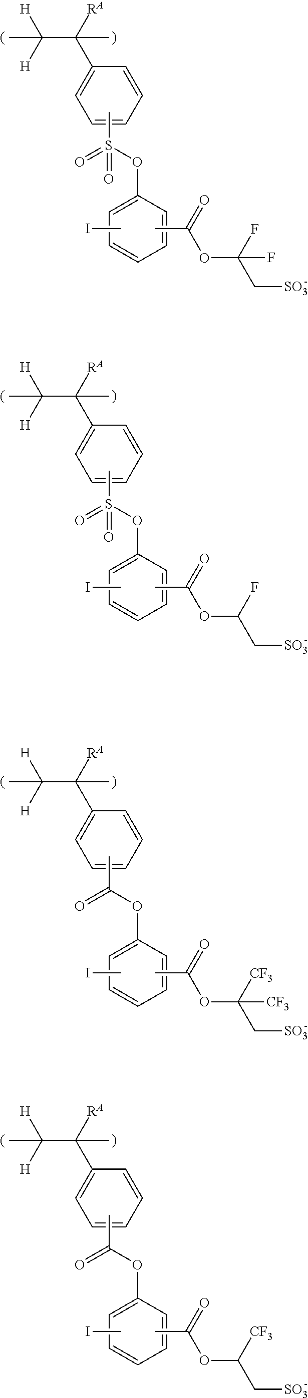

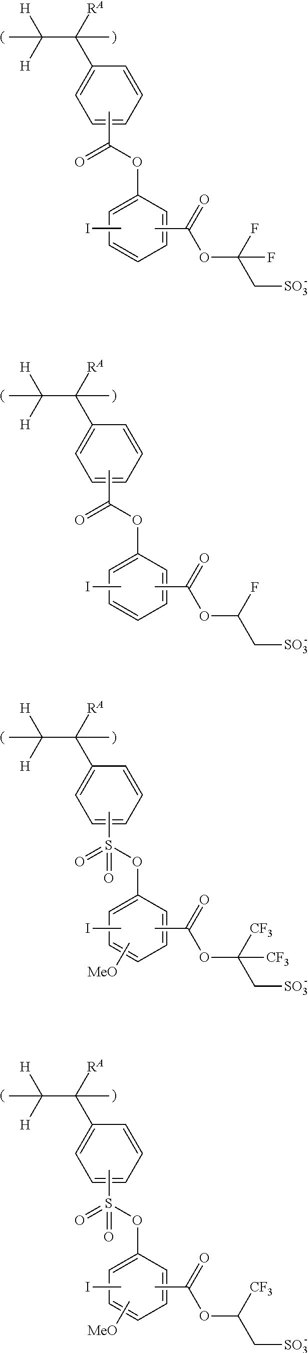

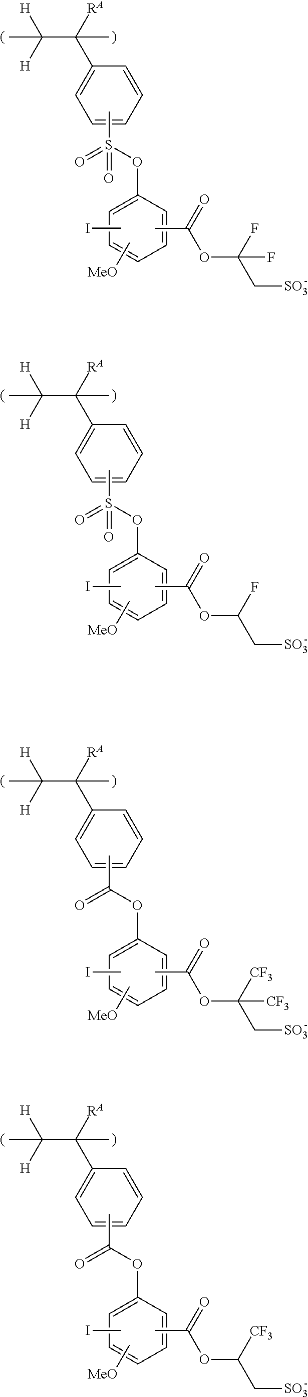

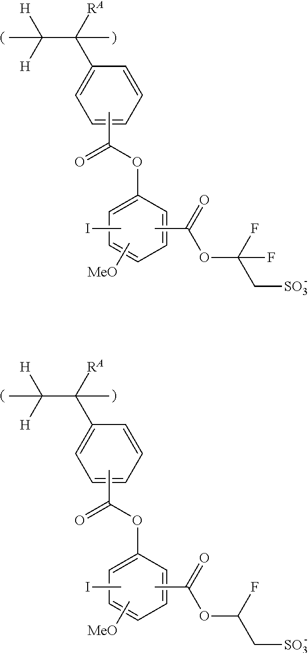

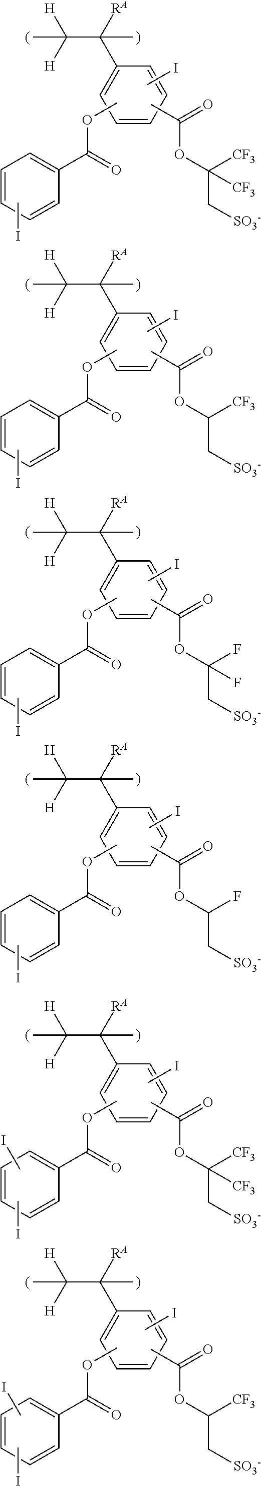

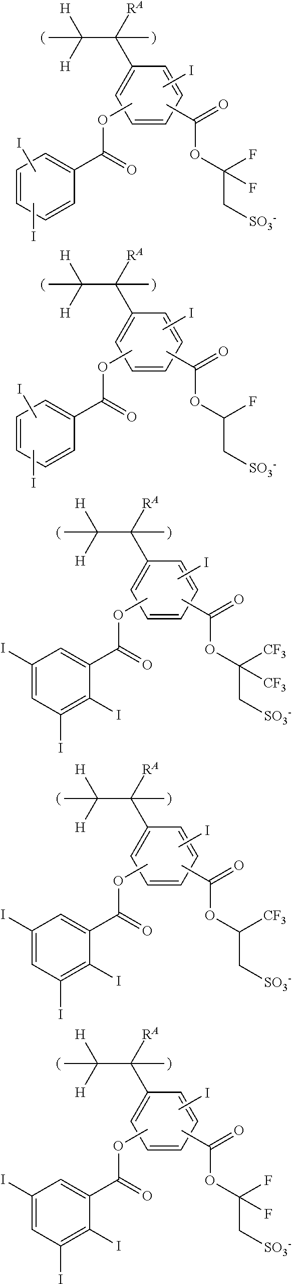

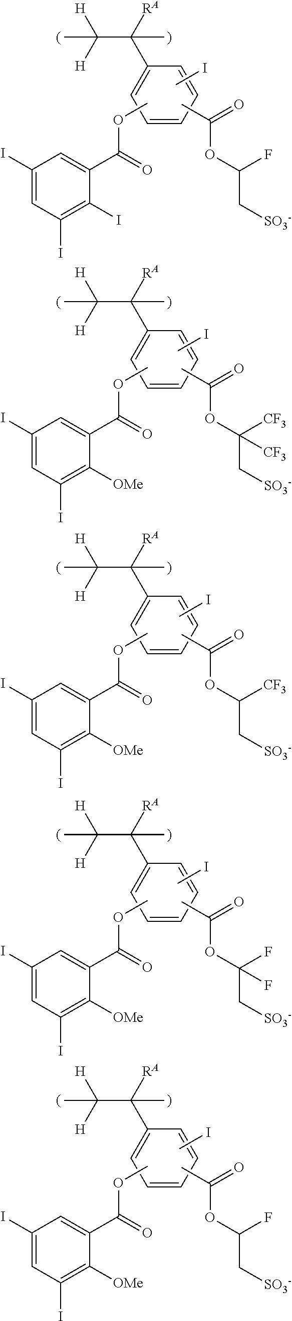

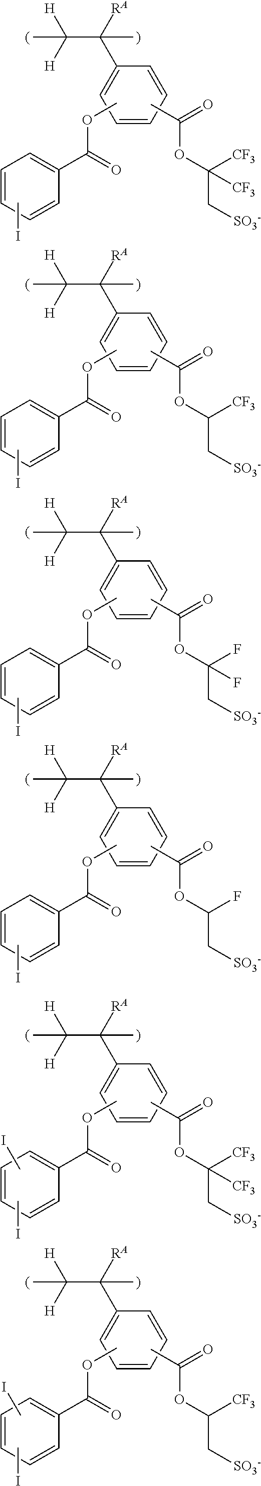

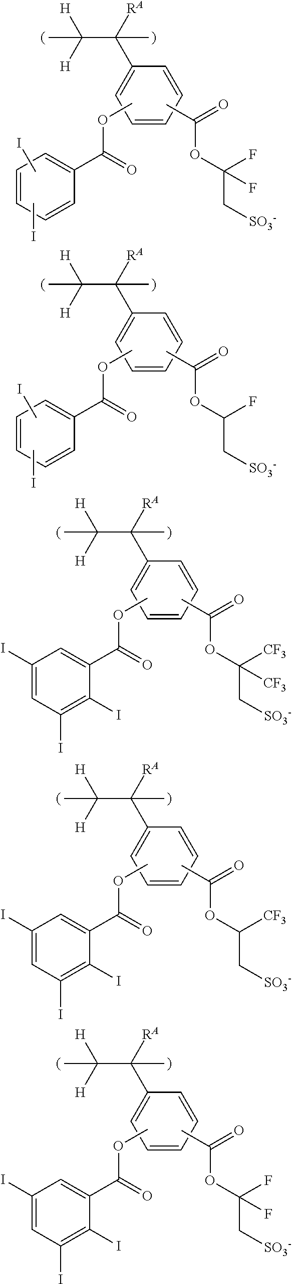

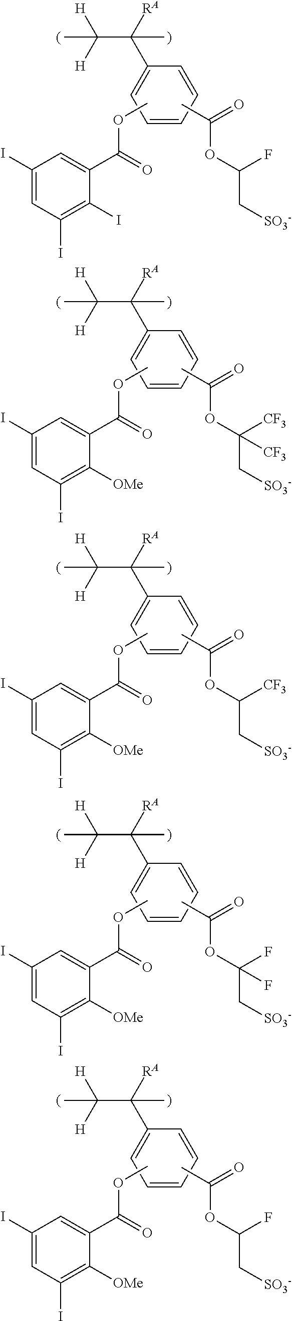

Examples of the anion in repeat unit (c4) include those shown below, but not limited thereto. In the formulae, R^{A} is as defined above.

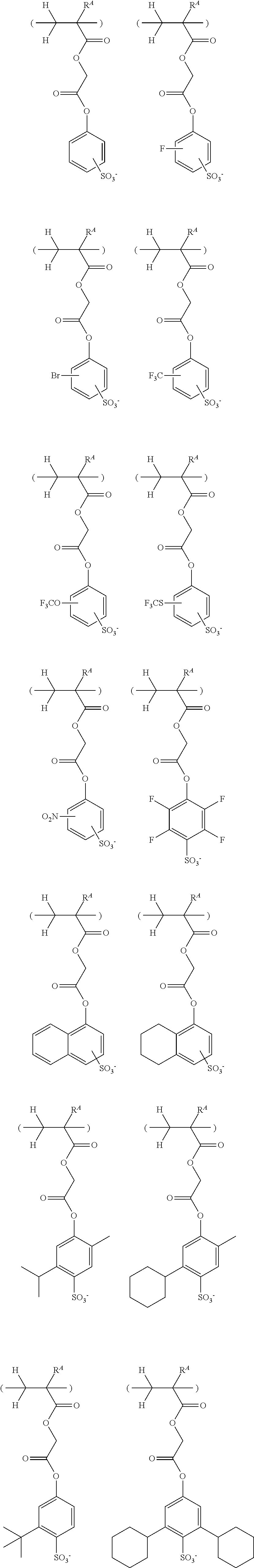

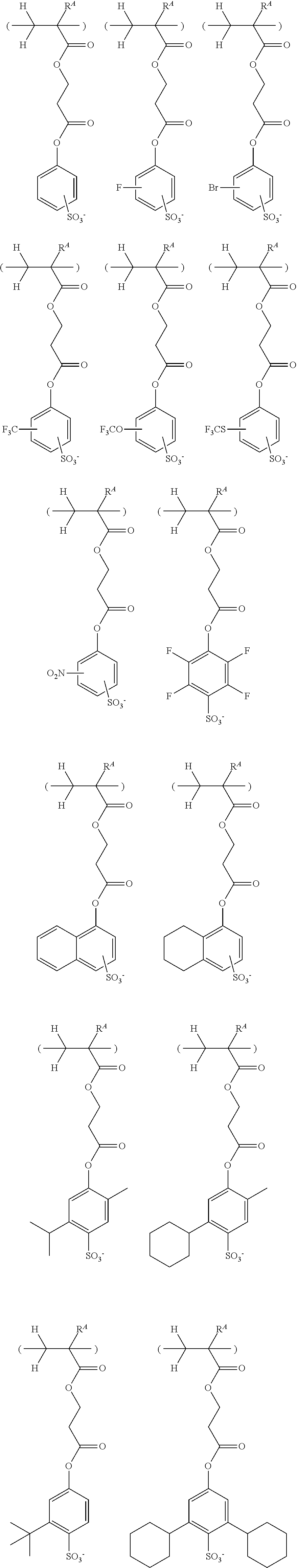

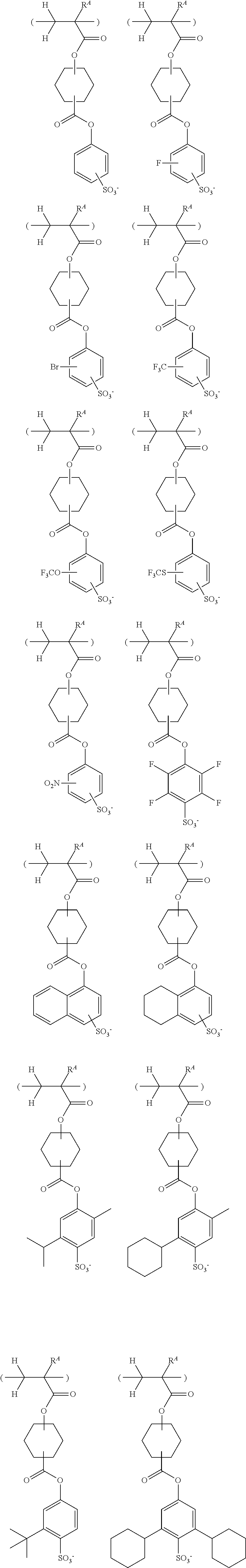

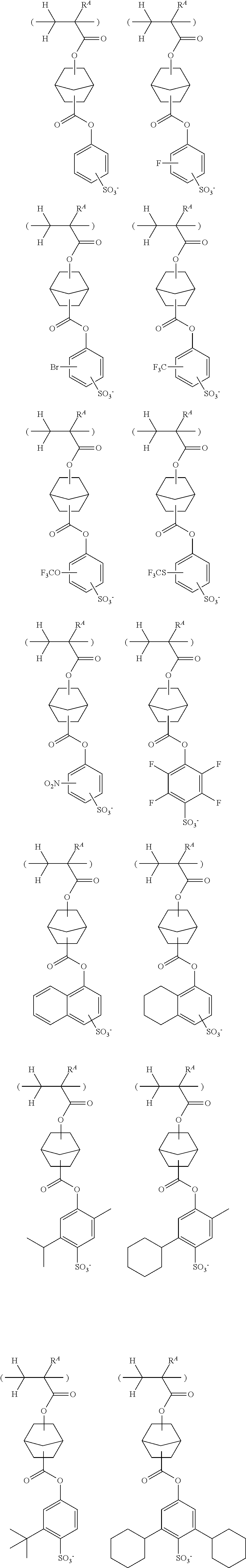

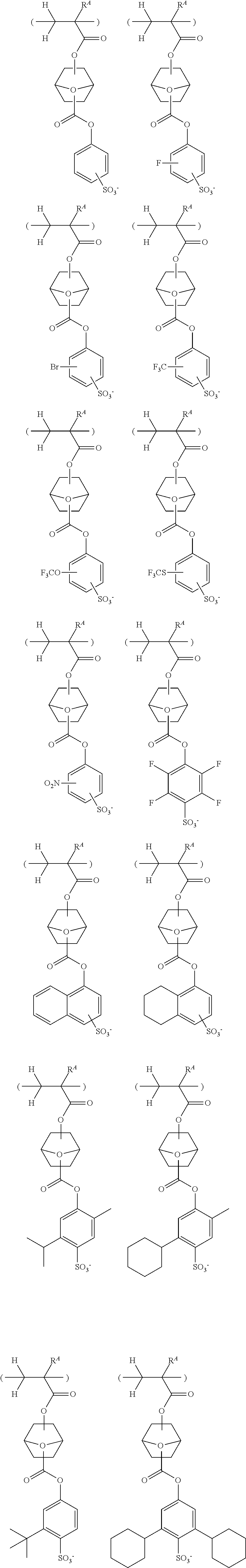

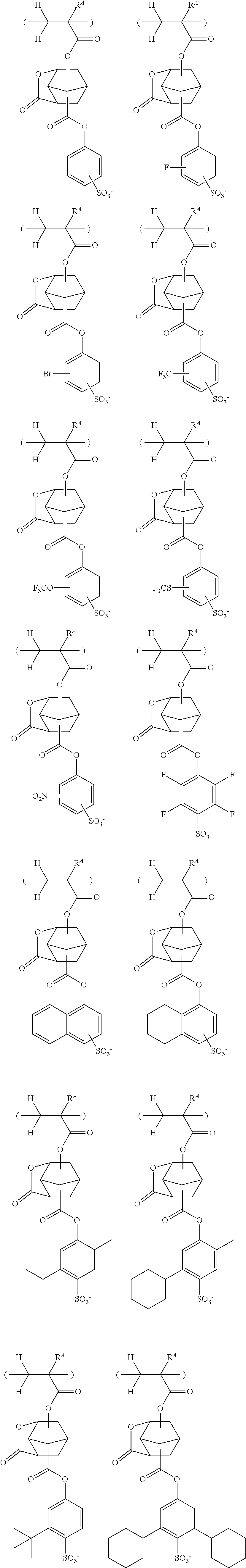

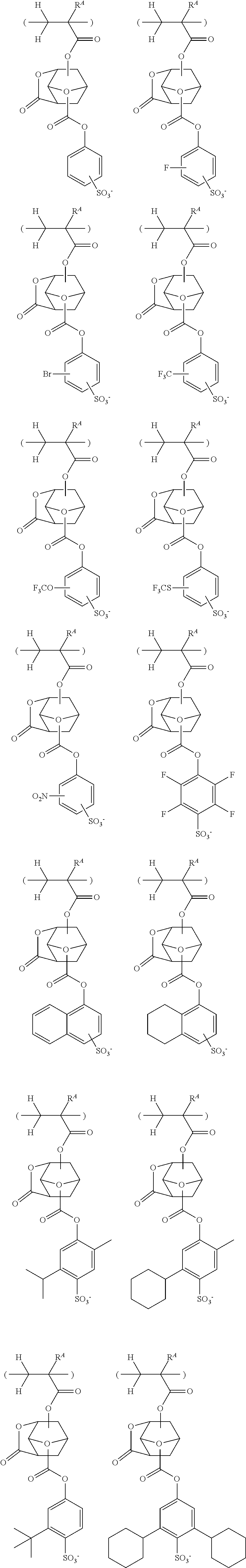

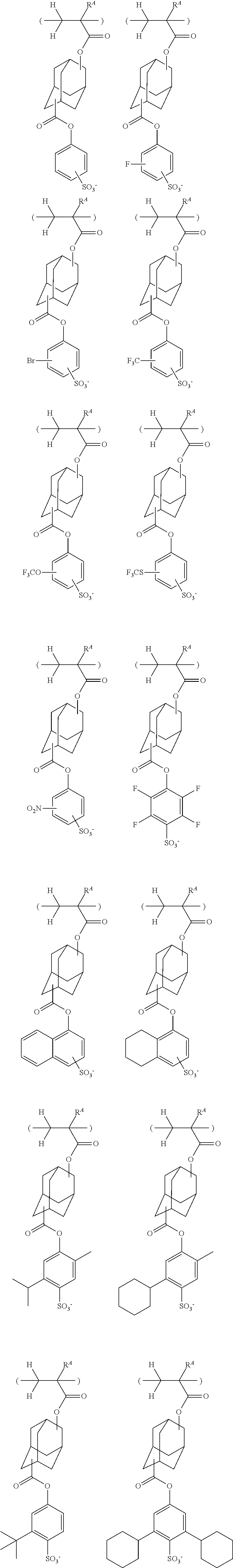

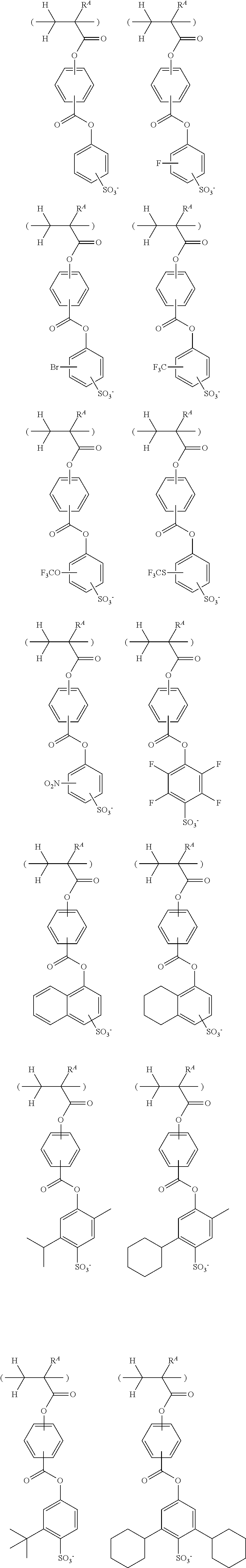

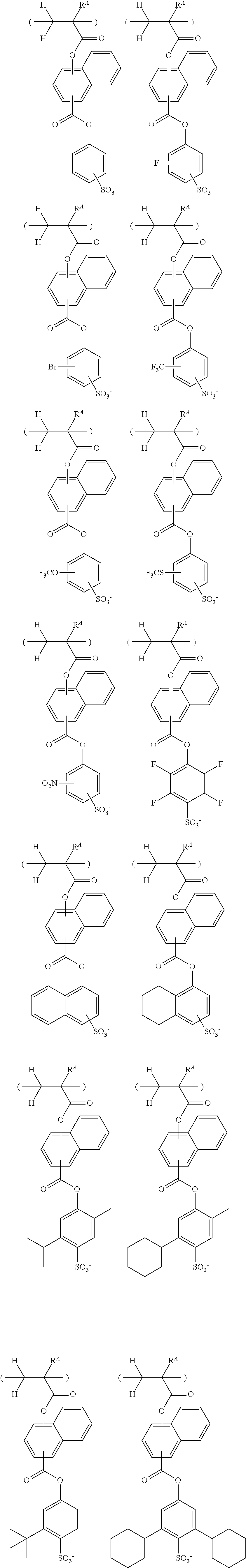

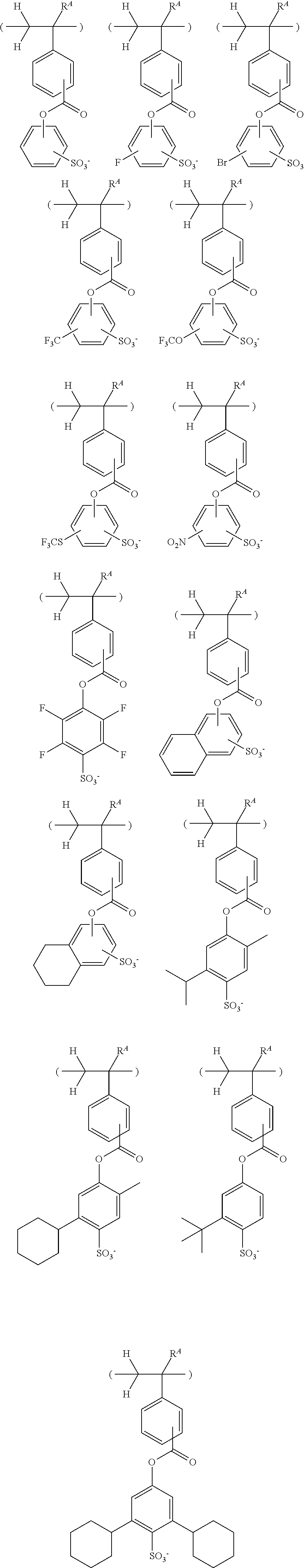

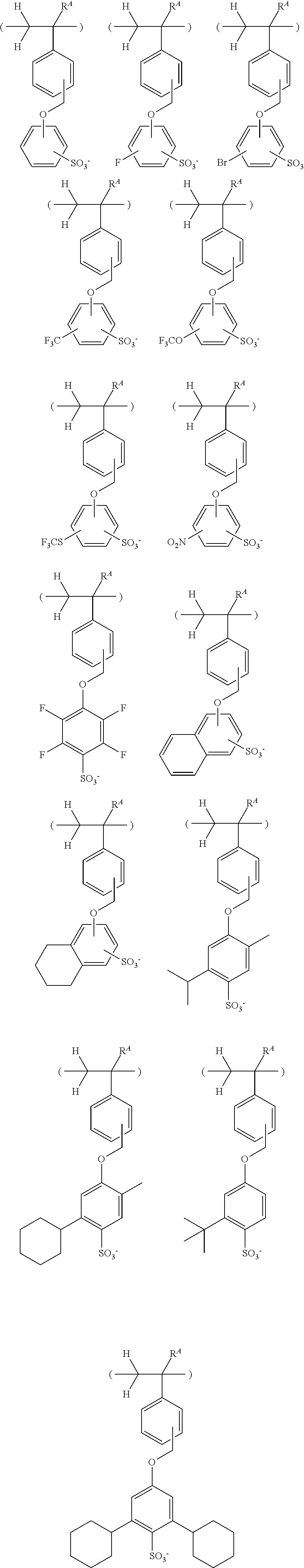

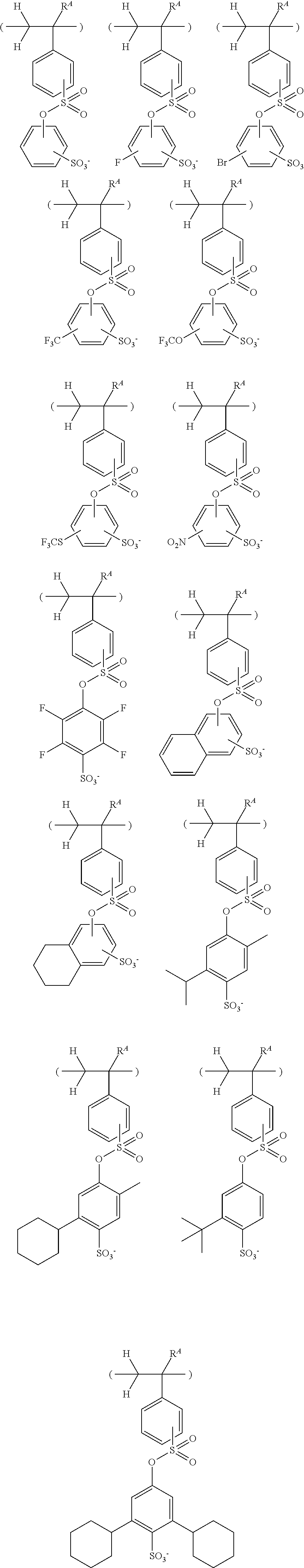

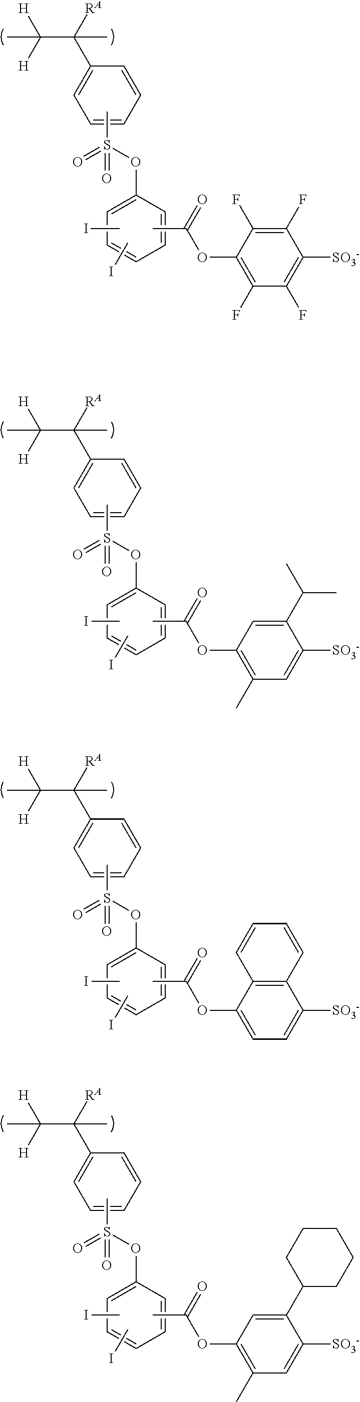

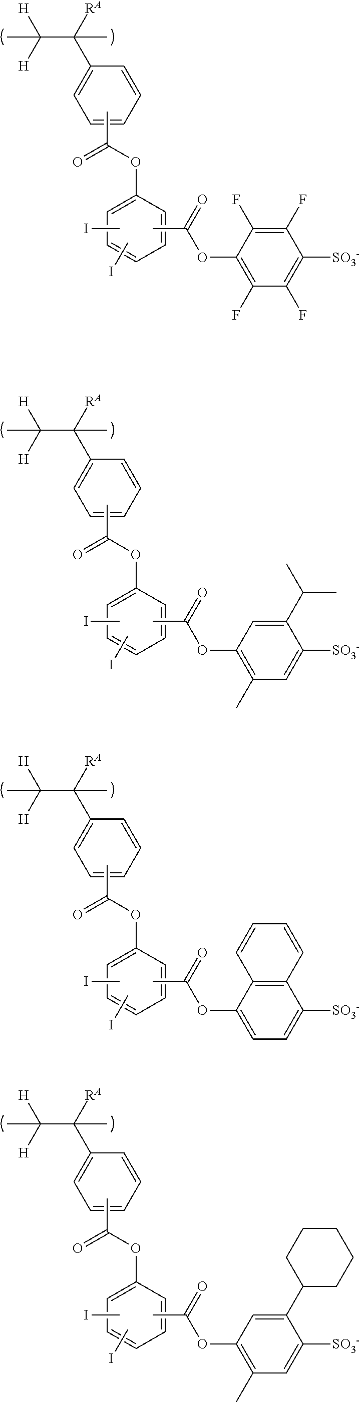

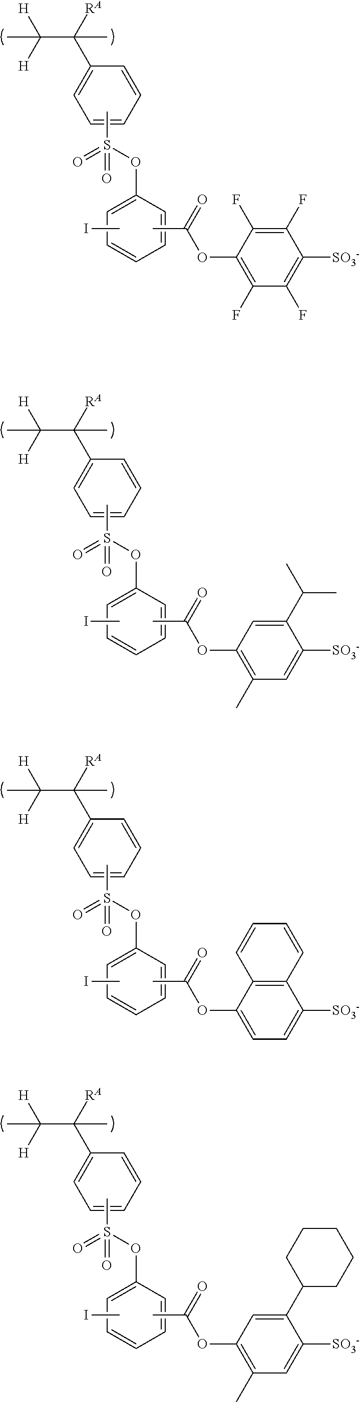

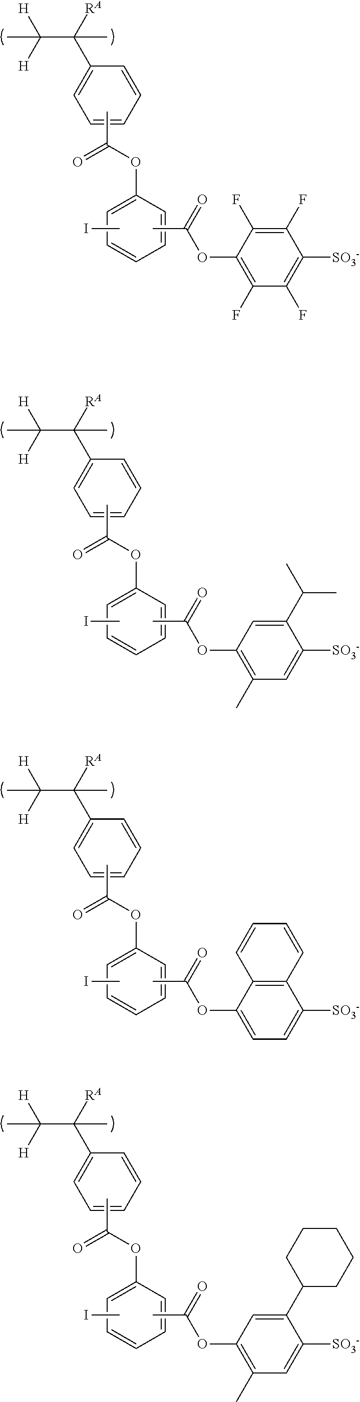

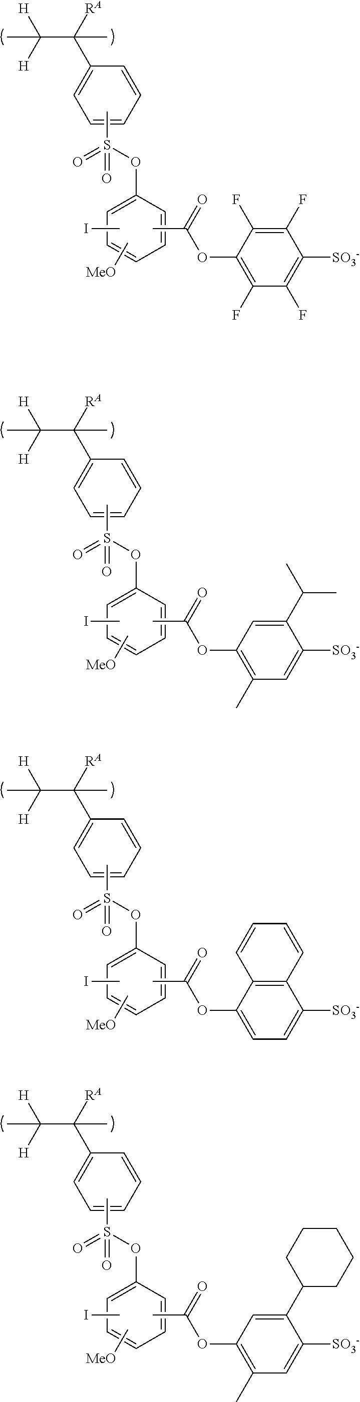

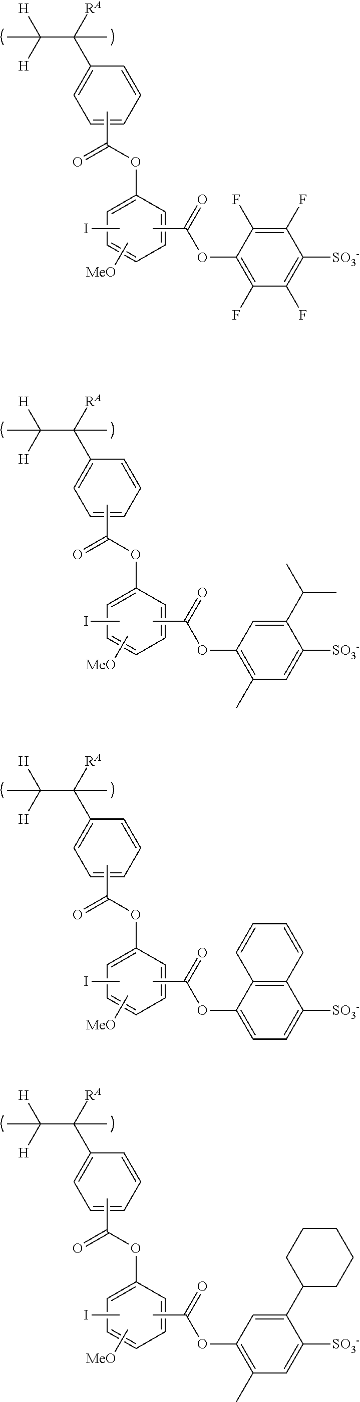

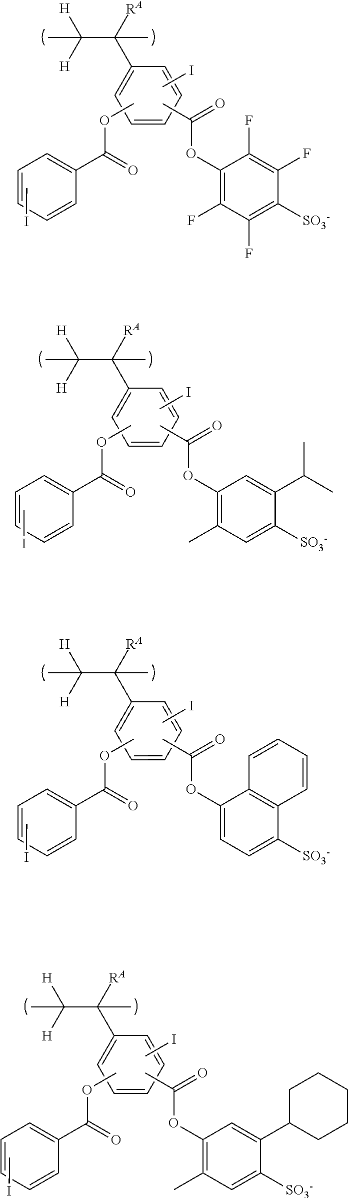

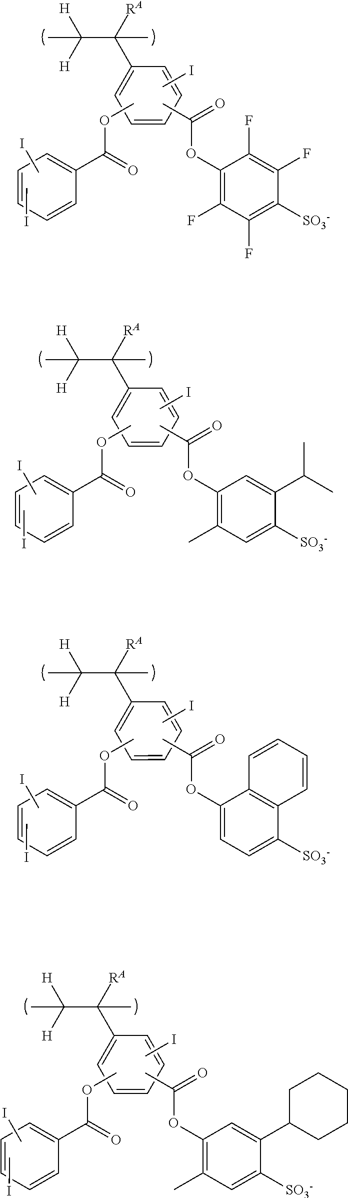

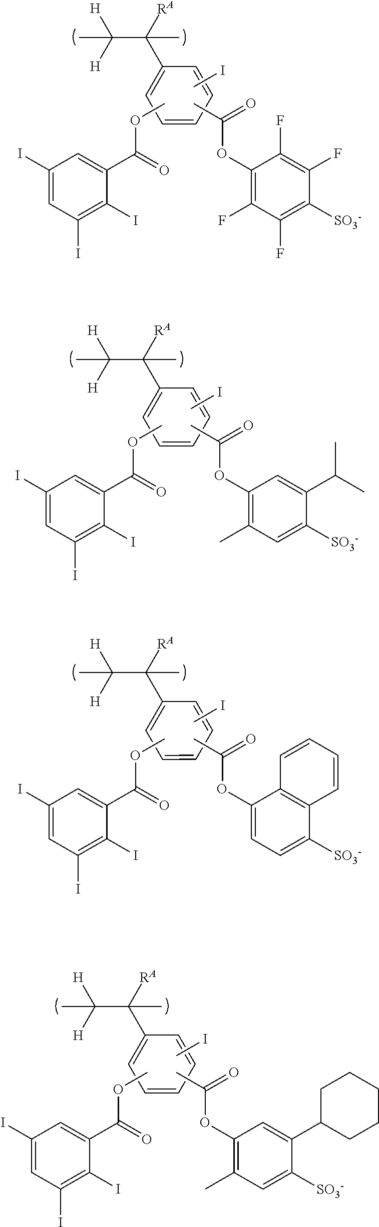

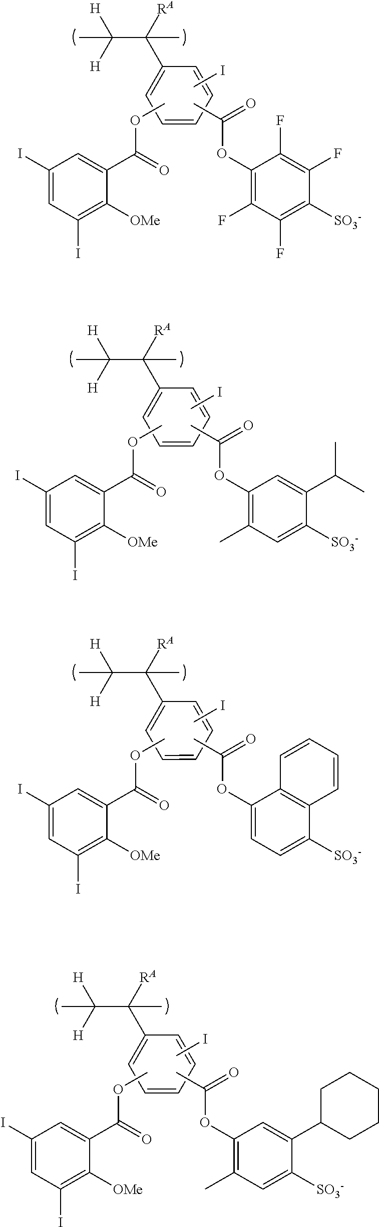

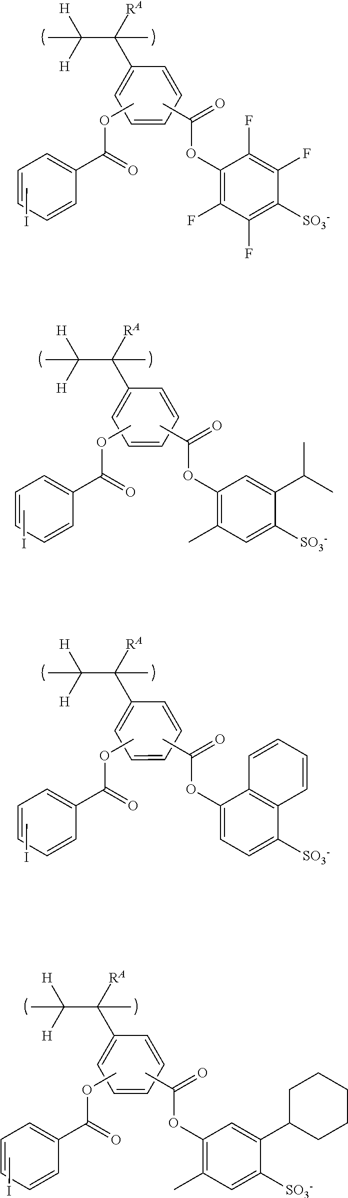

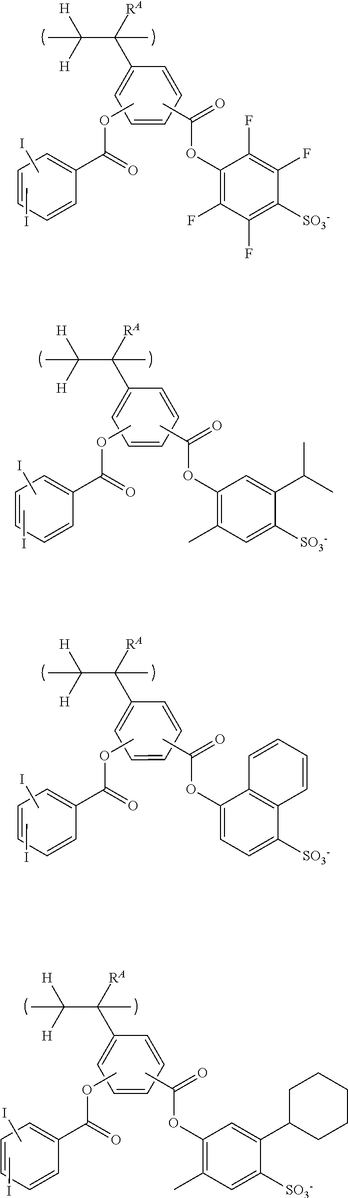

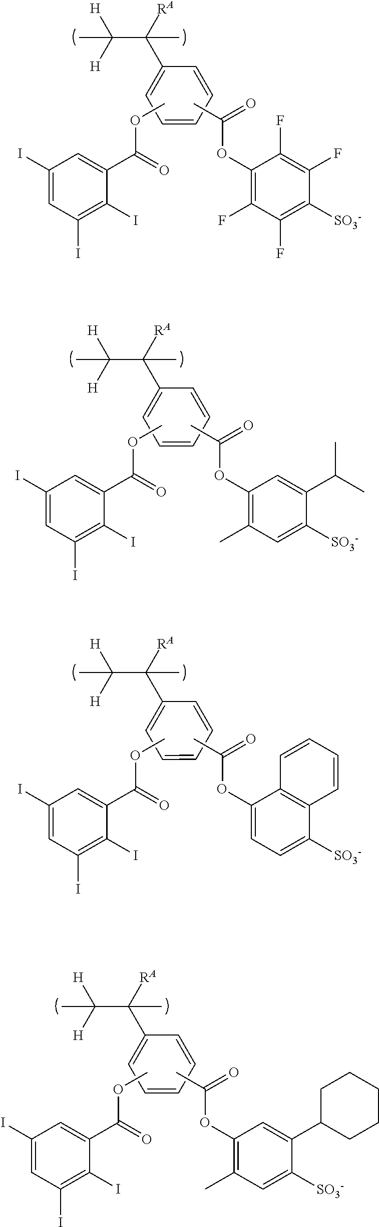

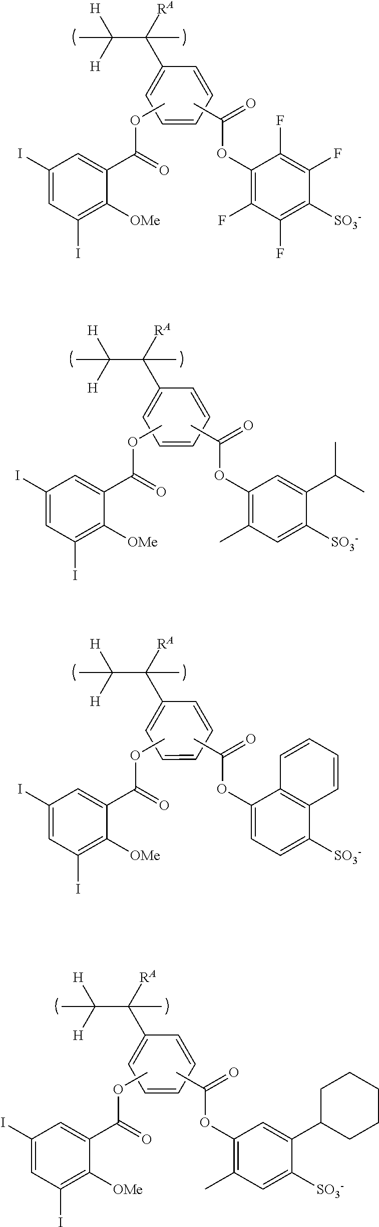

Examples of the anion in repeat unit (c5) include those shown below, but not limited thereto. In the formulae, R^{A} is as defined above.

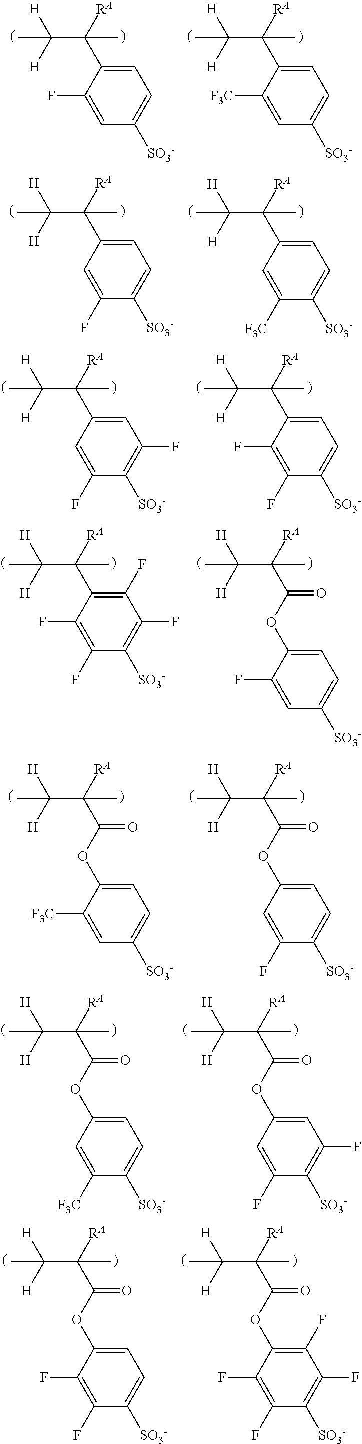

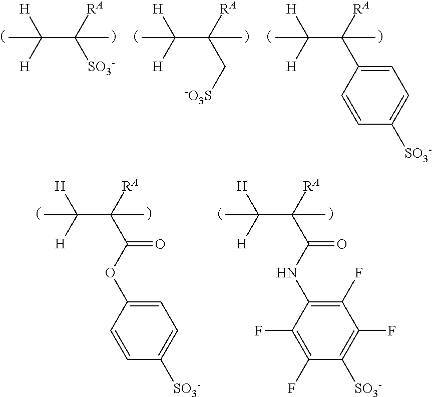

In the formulae (c2) to (c5), A⁺ is an onium cation. Examples of the onium cation include sulfonium, iodonium and ammonium cations, with sulfonium and iodonium cations being preferred. Examples of the sulfonium cation include those described as examples of the sulfonium cation in the formula (A), those described in JP-A 2024-003744, paragraphs [0102] to [0125], those described in WO 2024/128017, paragraphs [0044] to [0049], and those described in JP 7491173, paragraphs [0035] to [0046], but not limited thereto.

As the sulfonium cation, a sulfonium cation having the formula (sulfo-1) is also preferred.

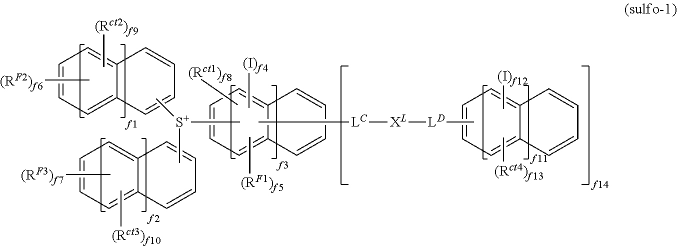

In the formula (sulfo-1), f1 is 0 or 1. The relevant structure is a benzene ring in case of f1=0, and a naphthalene ring in case of f1=1. The benzene ring corresponding to f1=0 is preferred from the aspect of solvent solubility. f2 is 0 or 1. The relevant structure is a benzene ring in case of f2=0, and a naphthalene ring in case of f2=1. The benzene ring corresponding to f2=0 is preferred from the aspect of solvent solubility. f3 is 0 or 1. The relevant structure is a benzene ring in case of f3=0, and a naphthalene ring in case of f3=1. The benzene ring corresponding to f3=0 is preferred from the aspect of solvent solubility.

In the formula (sulfo-1), f4 is 0, 1, 2, 3, or 4. A cation structure containing more iodine atoms is more absorptive to EUV, but loses solvent solubility so that it may precipitate in a resist composition, f4 is preferably 0, 1, 2, or 3, more preferably 0, 1, or 2.

In the formula (sulfo-1), f5 is 0, 1, 2, 3, or 4. f5 is preferably 0, 1, 2, or 3, more preferably 0, 1, or 2 from the aspect of availability of reactants. f6 is 0, 1, 2, 3, 4, 5, or 6. f6 is preferably 0, 1, 2, or 3, more preferably 0, 1, or 2 from the aspect of availability of reactants. f is 0, 1, 2, 3, 4, 5, or 6. f7 is preferably 0, 1, 2, or 3, more preferably 0, 1, or 2 from the aspect of availability of reactants.

In the formula (sulfo-1), f8 is 0, 1, or 2. f8 is preferably 0 or 1 from the aspect of availability of reactants. f9 is 0, 1, or 2. f9 is preferably 0 or 1 from the aspect of availability of reactants. f10 is 0, 1, or 2. f10 is preferably 0 or 1 from the aspect of availability of reactants.

In the formula (sulfo-1), f11 is 0 or 1. The relevant structure is a benzene ring in case of f11=0, and a naphthalene ring in case of f11=1. The benzene ring corresponding to f11=0 is preferred from the aspect of solvent solubility.

In the formula (sulfo-1), f12 is 0, 1, 2, 3, or 4. A cation structure containing more iodine atoms is more absorptive to EUV, but loses solvent solubility so that it may precipitate in a resist composition, f12 is preferably 0, 1, 2, or 3, more preferably 0, 1, or 2.

In the formula (sulfo-1), f13 is 0, 1, or 2. f13 is preferably 0 or 1 from the aspect of availability of reactants. f14 is 0, 1, or 2. From the aspect of synthesis, f14 is preferably 0 or 1.

It is noted that 0 f6+f9 4 in case of f1=0 and 0≤f6+f9≤6 in case of f1=1; 0≤f7+f10≤4 in case of f2=0 and 0≤f7+f10≤6 in case of f2=1; 1≤f4+f5+f8+f14≤4 in case of f3=0 and 1≤f4+f5+f8+f14≤6 in case of f3=1; 0≤f12+f13≤4 in case of f11=0 and 0≤f12+f13≤6 in case of f11=1; and f4+f12≥1.

In the formula (sulfo-1), R^{F1} to R^{F3} are each independently fluorine, a C₁-C₆ fluorinated saturated hydrocarbyl group, a C₁-C₆ fluorinated saturated hydrocarbyloxy group, or a C₁-C₆ fluorinated saturated hydrocarbylthio group. Among them, R^{F1} to R^{F3} are preferably a trifluoromethyl, trifluoromethoxy or trifluorothiomethoxy group. A plurality of R^{F1} may be identical or different when f5 is 2, 3, or 4, a plurality of R^{F2} may be identical or different when f6 is 2, 3, 4, 5, or 6, and a plurality of R^{F3} may be identical or different when f7 is 2, 3, 4, 5, or 6.

In the formula (sulfo-1), R^{ct1} to R^{ct4} are halogen exclusive of iodine and fluorine, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and hydrocarbyl moiety in the hydrocarbyloxy and hydrocarbylthio groups may be saturated or unsaturated and straight, branched or cyclic. Examples thereof include those exemplified above for the hydrocarbyl group represented by R¹ in the formula (1). In the hydrocarbyl group and hydrocarbyl moiety in the hydrocarbyloxy and hydrocarbylthio groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

When f8 is 2, two R^{ct1} may be identical or different and two R^{ct1} may bond together to form a ring with the carbon atoms to which they are attached; when f9 is 2, two R^{ct2} may be identical or different and two R^{ct2} may bond together to form a ring with the carbon atoms to which they are attached; when f10 is 2, two R^{ct3} may be identical or different and two R^{ct3} may bond together to form a ring with the carbon atoms to which they are attached; and when f13 is 2, two R^{ct4} may be identical or different and two R^{ct4} may bond together to form a ring with the carbon atoms to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

The aromatic rings directly bonded to S⁺ in the sulfonium cation having the formula (sulfo-1) may bond together to form a ring with S⁺. Examples of the structure of the ring include structures having the formulae:

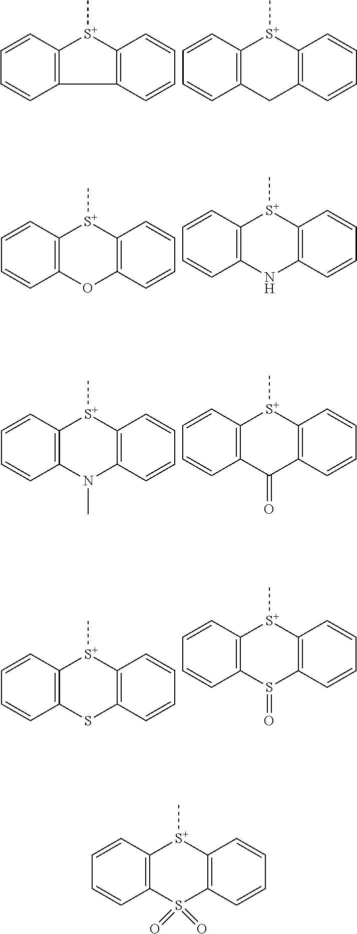

wherein the broken line is a valence bond.

In the formula (sulfo-1), L^{C} and L^{D} are each independently a single bond, ether bond, ester bond, amide bond, sulfonate ester bond, sulfonamide bond, carbonate bond or carbamate bond. Of these, L^{C} is preferably a single bond, ether bond, ester bond or sulfonate ester bond, more preferably an ester bond or sulfonate ester bond. L^{D} is preferably a single bond, ether bond or ester bond, more preferably a single bond.

In the formula (sulfo-1), X^{L} is a single bond or a C₁-C₄₀ hydrocarbylene group which may contain a heteroatom. The hydrocarbylene group may be straight, branched or cyclic, and examples thereof include alkanediyl, cyclic saturated hydrocarbylene and arylene groups. Examples of the heteroatom include oxygen, nitrogen and sulfur atoms.

Examples of the C₁-C₄₀ hydrocarbylene group which may contain a heteroatom represented by X^{L} include those shown below, but not limited thereto. In the formulae, the asterisk (*) is a point of attachment to L^{C} and L^{D}, respectively.

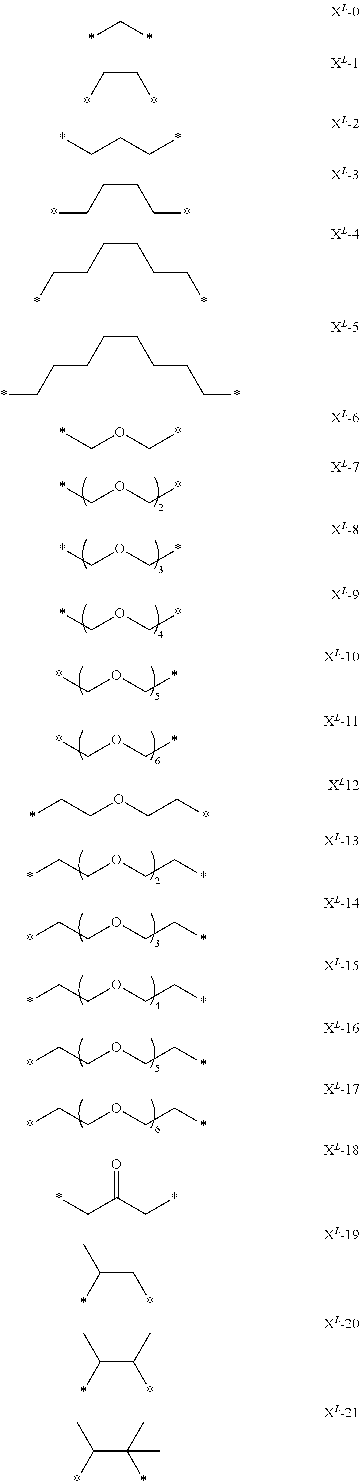

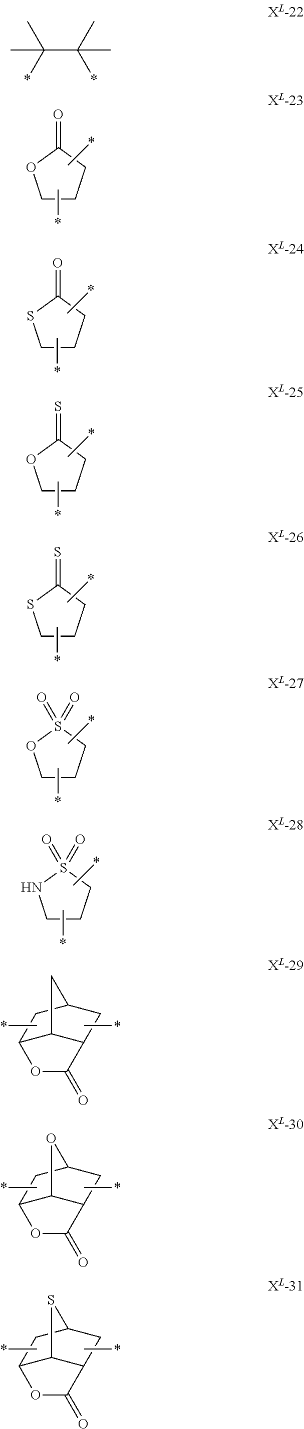

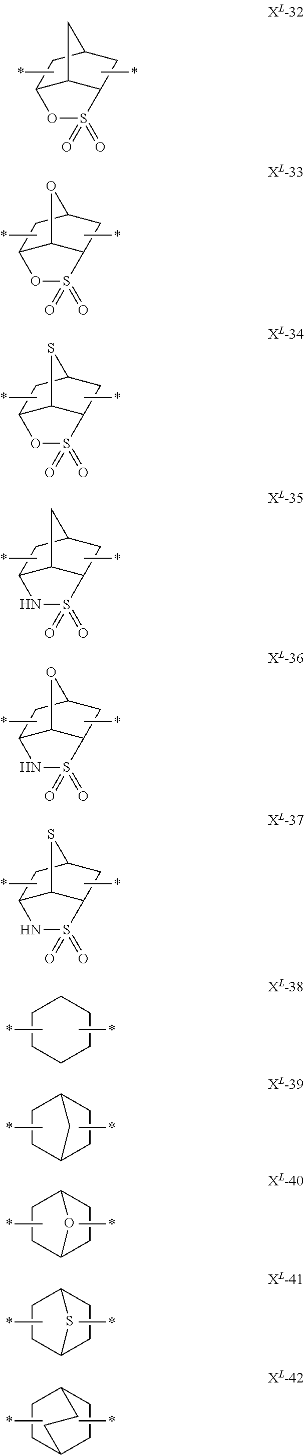

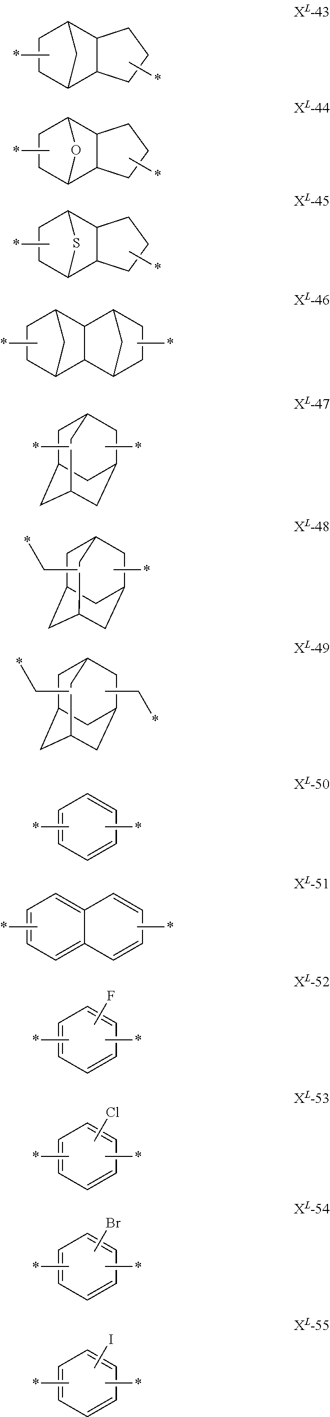

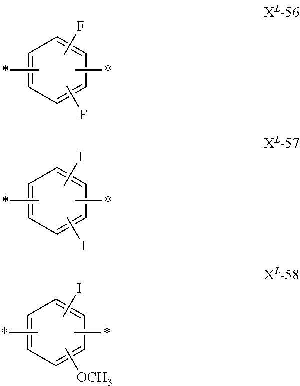

Of these, X^{L}-0 to X^{L}-22, X^{L}-29 to X^{L}-34, and X^{L}-47 to X^{L}-58 are preferred.

As the sulfonium cation having the formula (sulfo-1), a sulfonium cation having the formula (sulfo-1-1) is preferred.

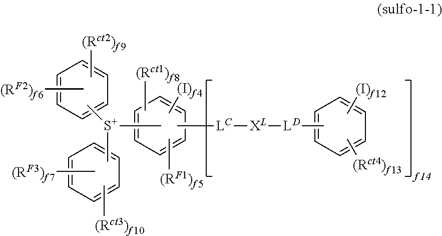

Herein f4 to f10, f12 to f14, R^{F1} to R^{F3}, R^{ct1} to R^{ct4}, L^{C}, L^{D} and X^{L} are as defined above.

As the sulfonium cation having the formula (sulfo-1-1), a sulfonium cation having the formula (sulfo-1-2) is preferred.

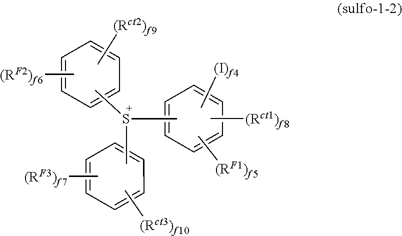

Herein f4 to f10, R^{F1} to R^{F3} and R^{ct1} to R^{ct3} are as defined above.

Examples of the sulfonium cation represented by the formula (sulfo-1) include those shown below, but not limited thereto. In the formulae, Me is methyl.

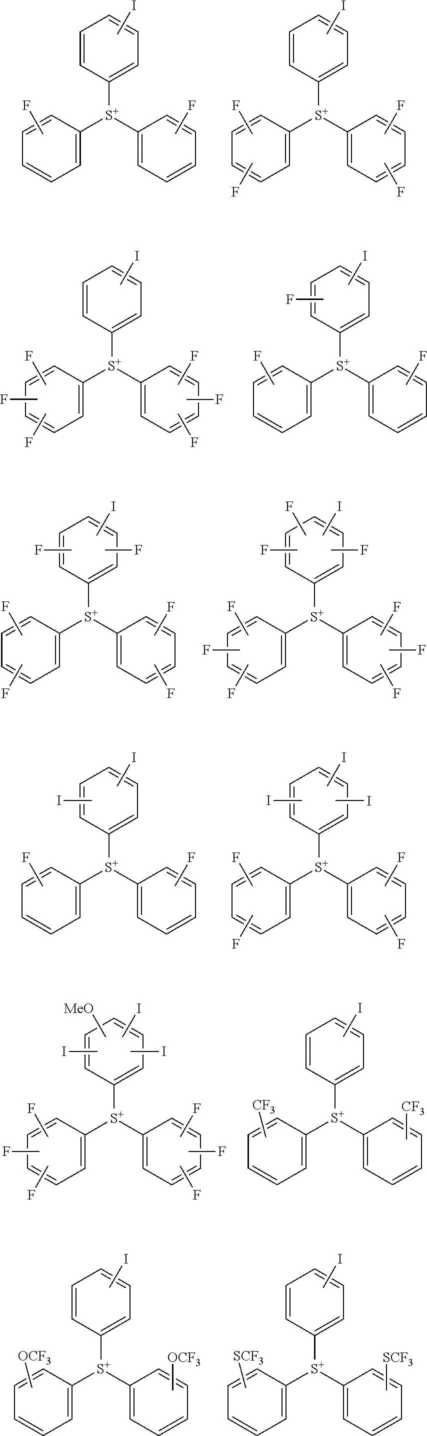

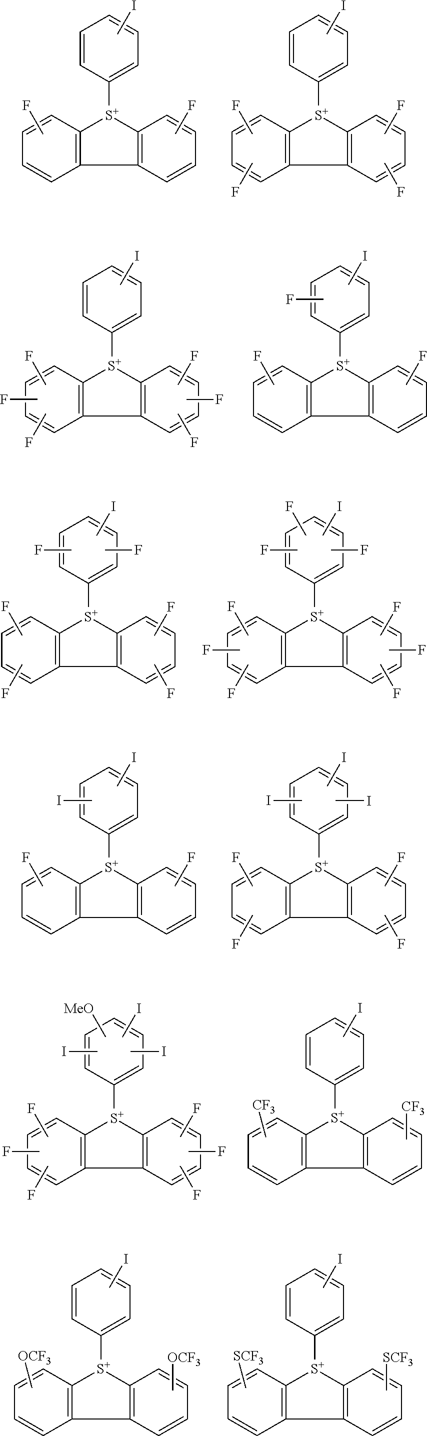

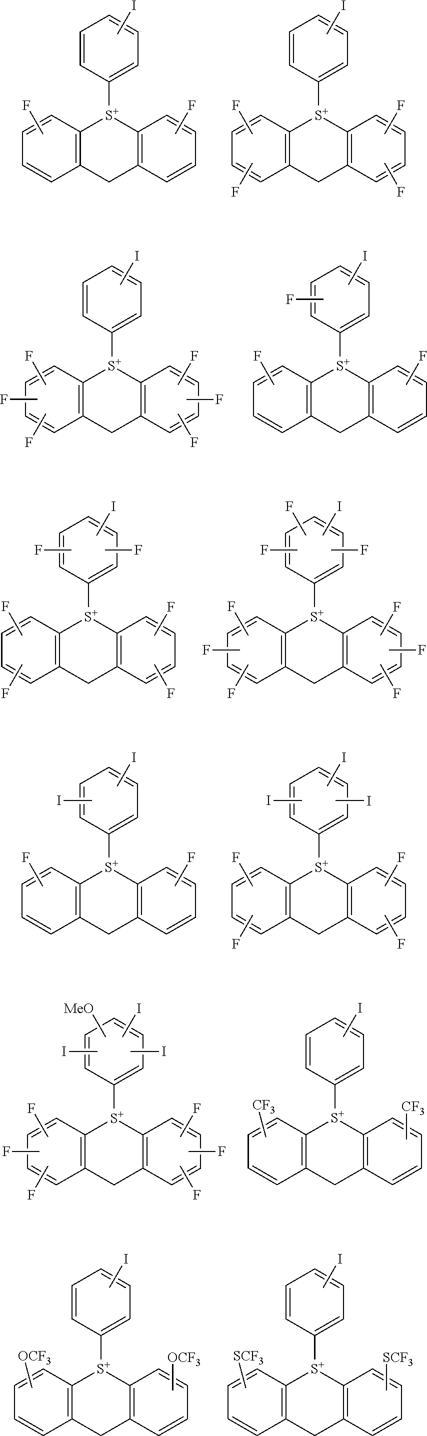

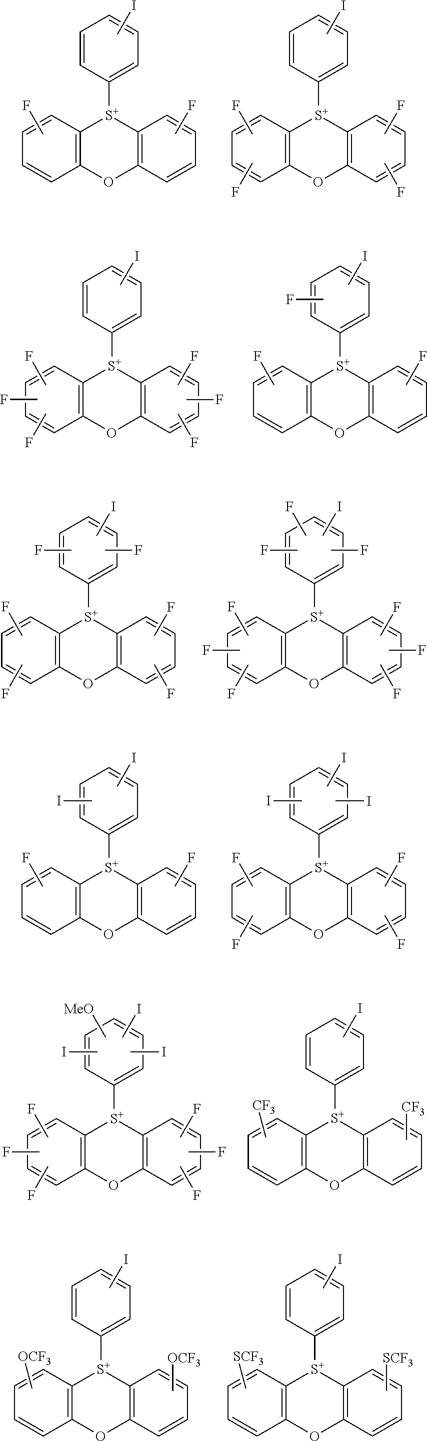

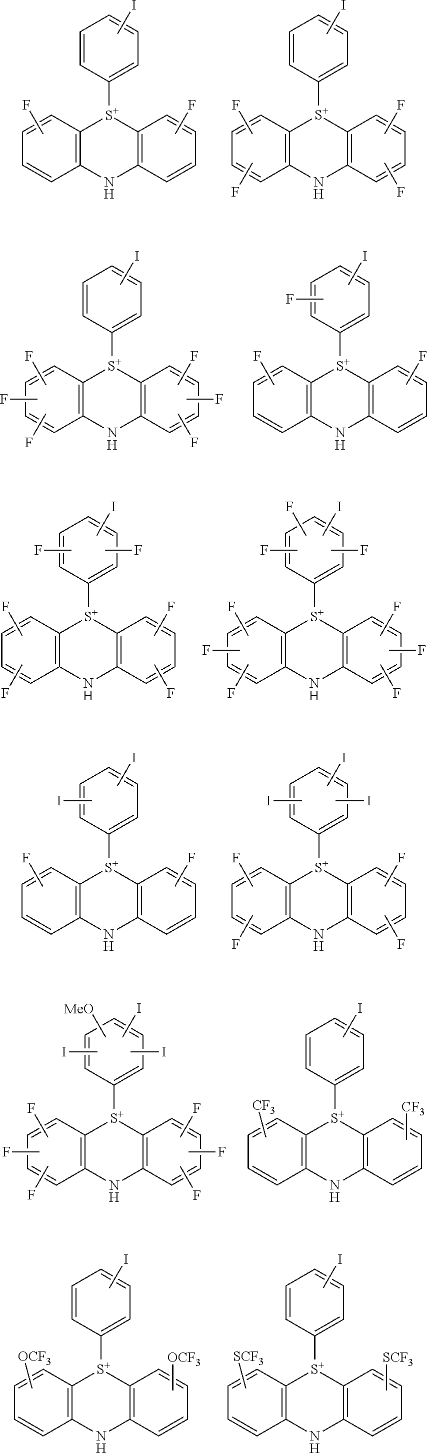

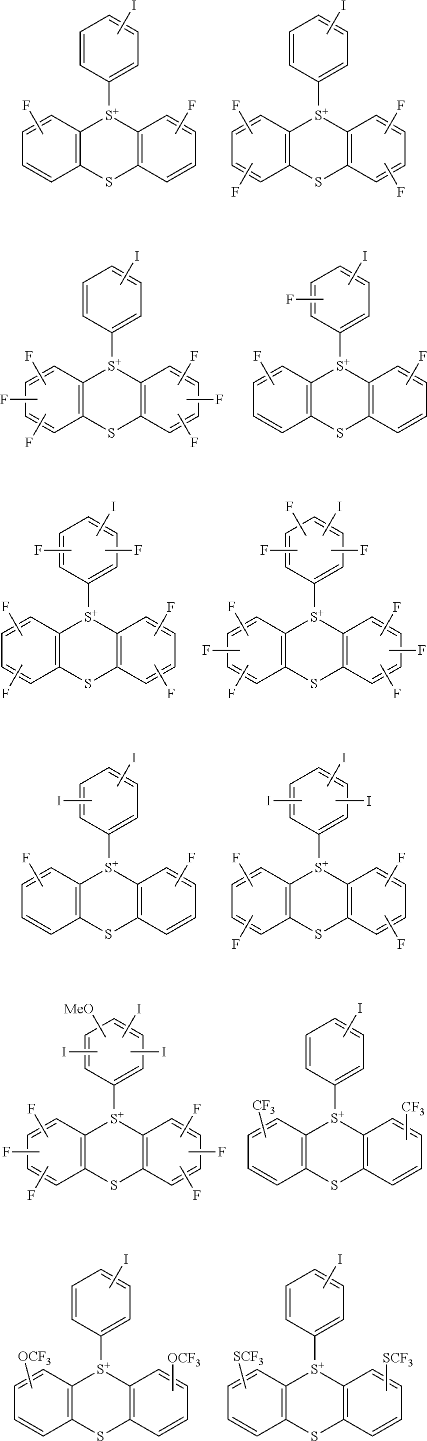

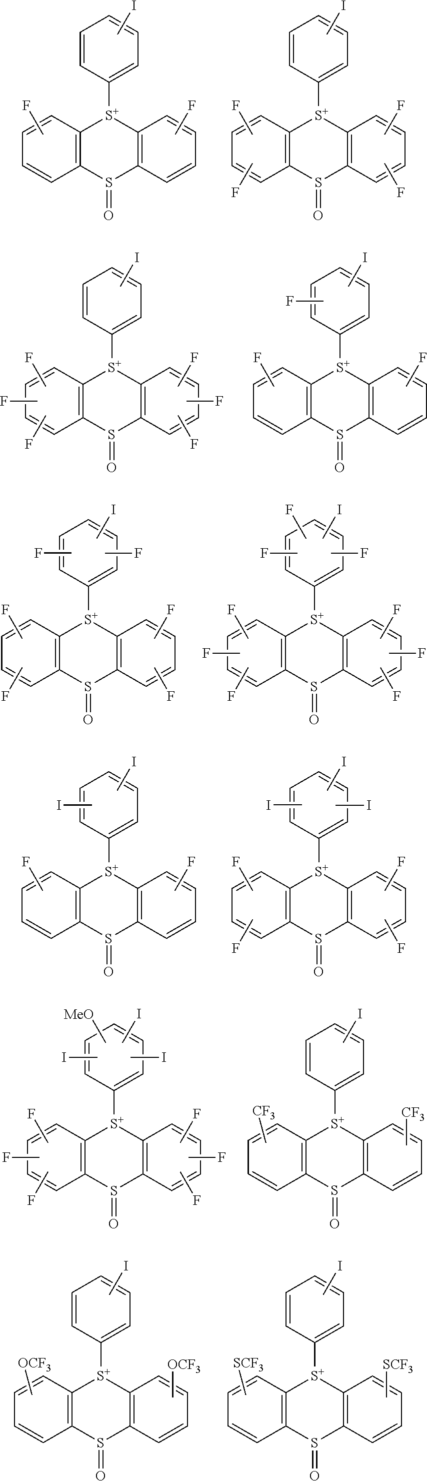

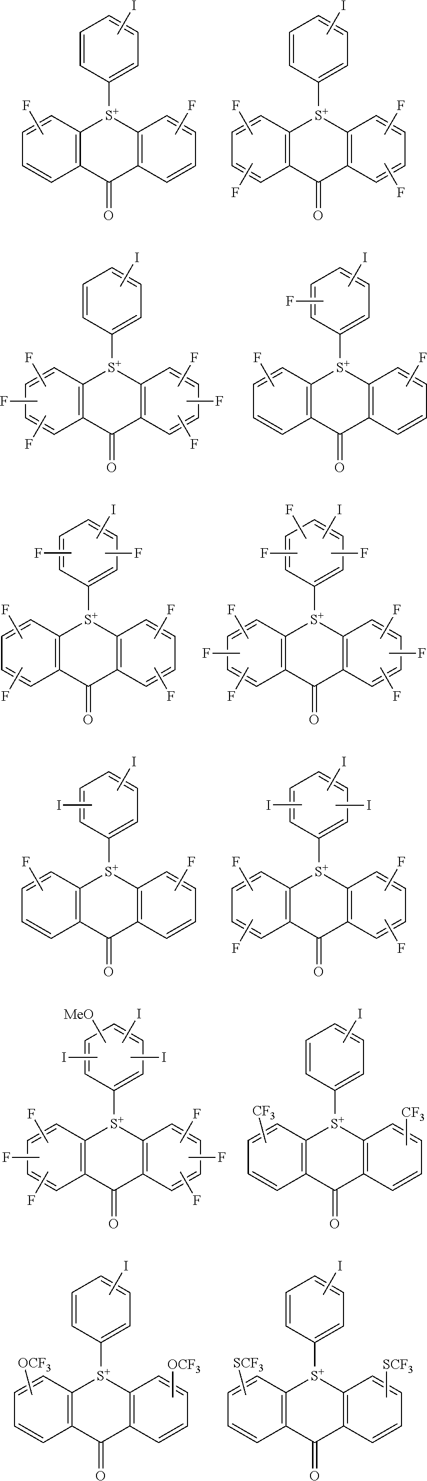

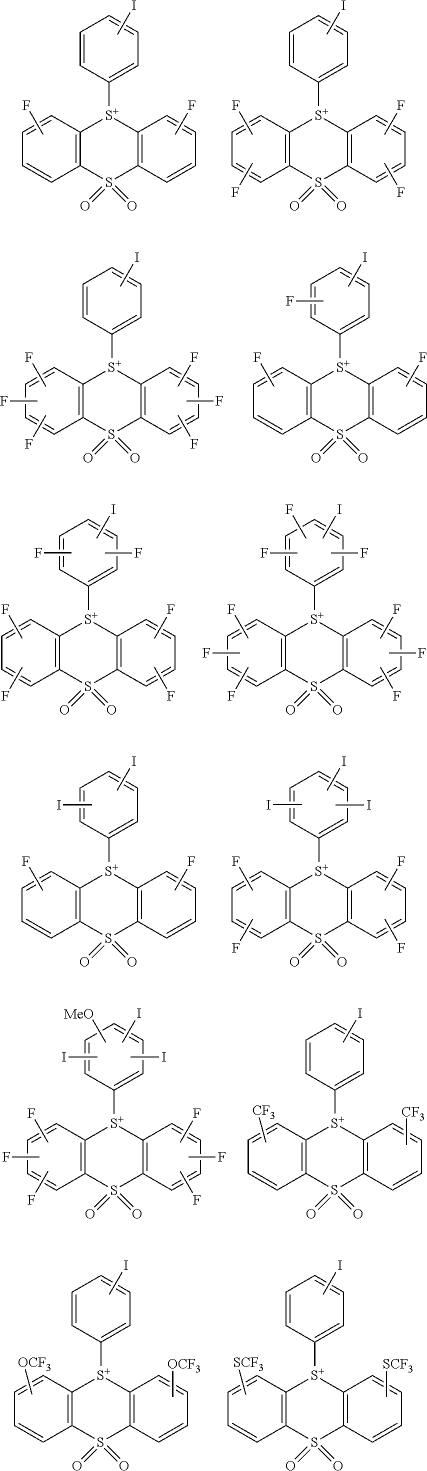

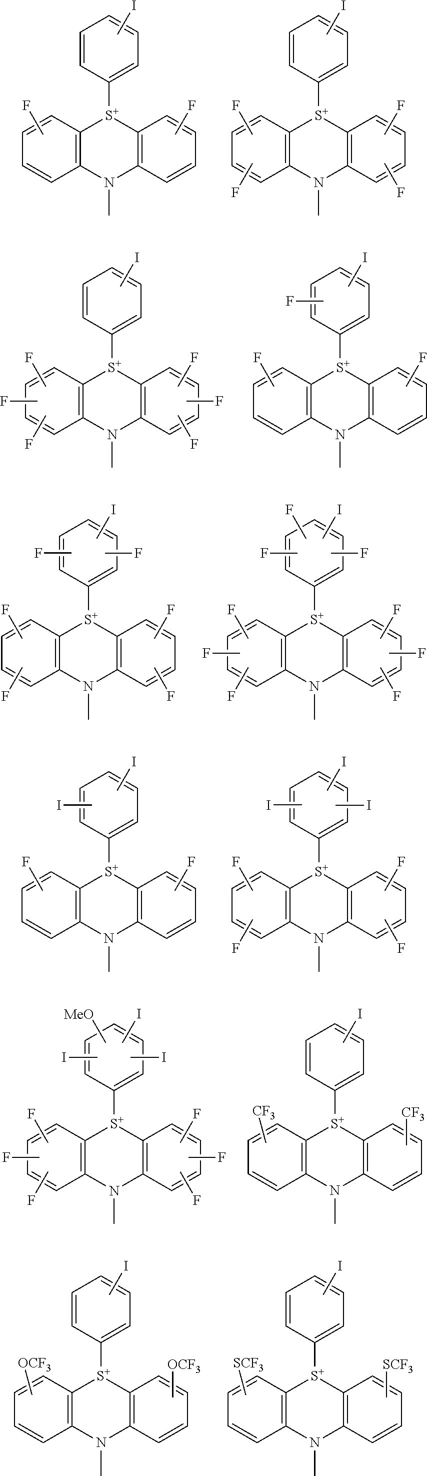

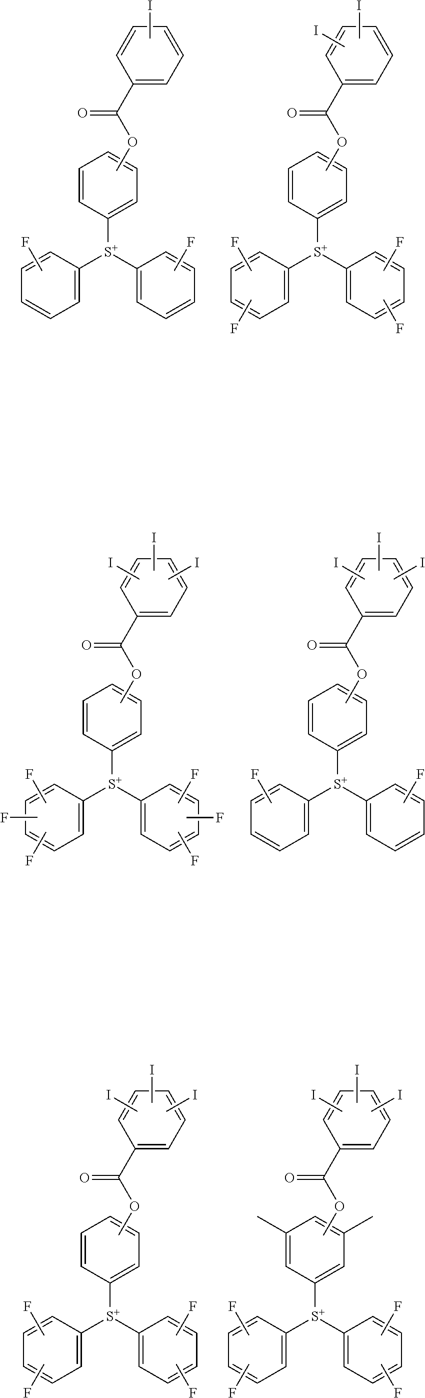

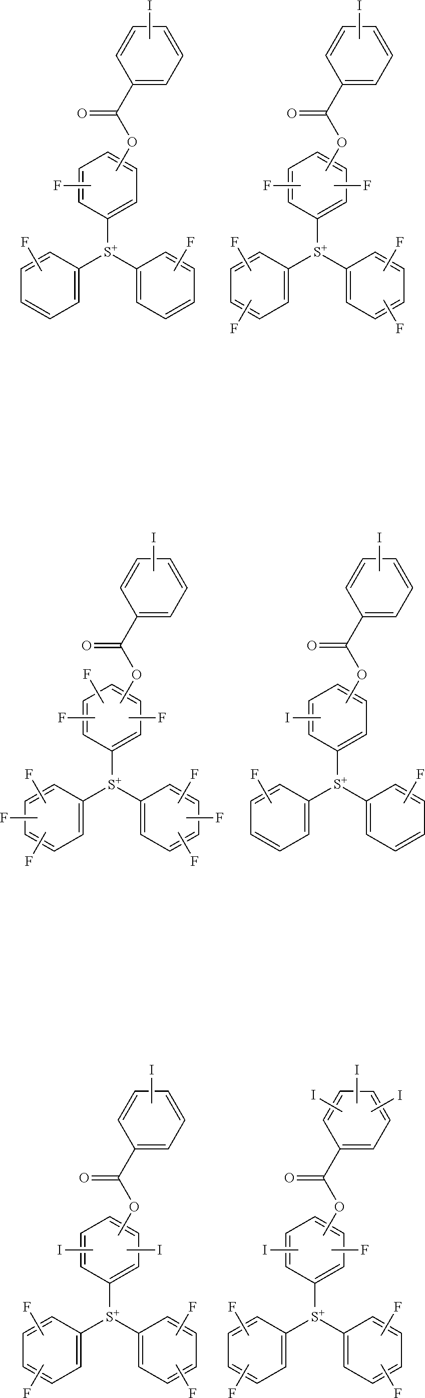

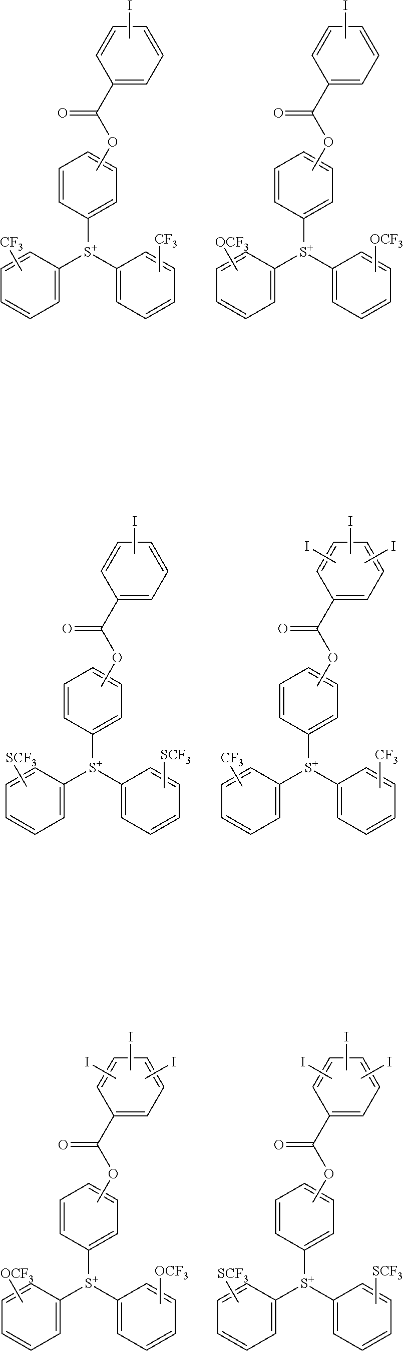

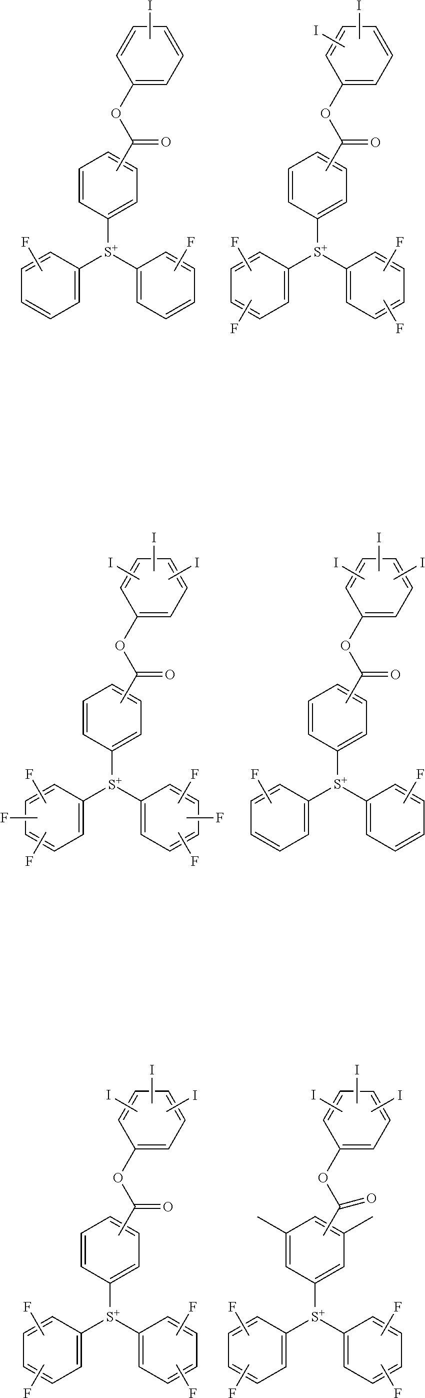

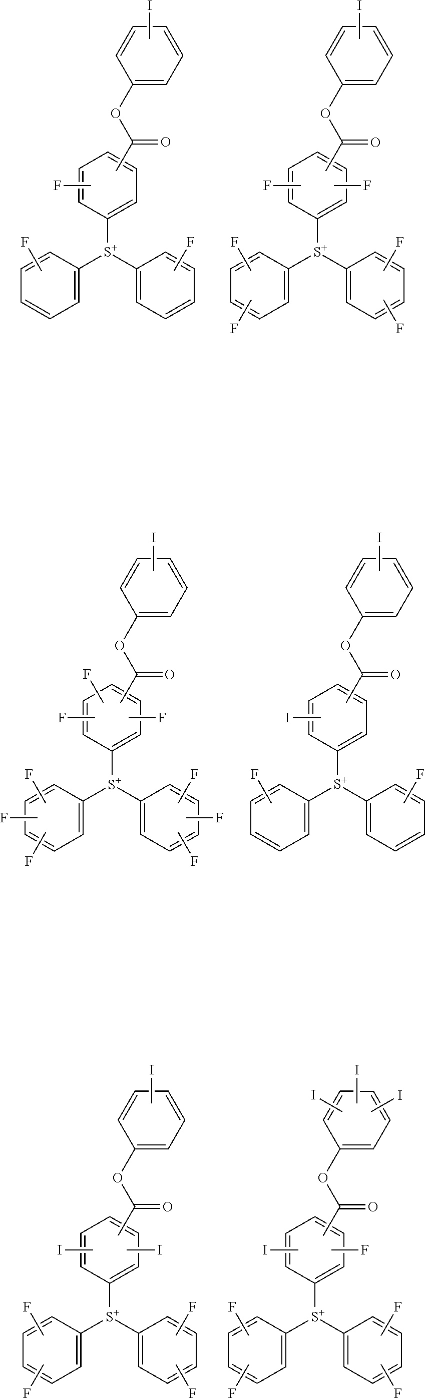

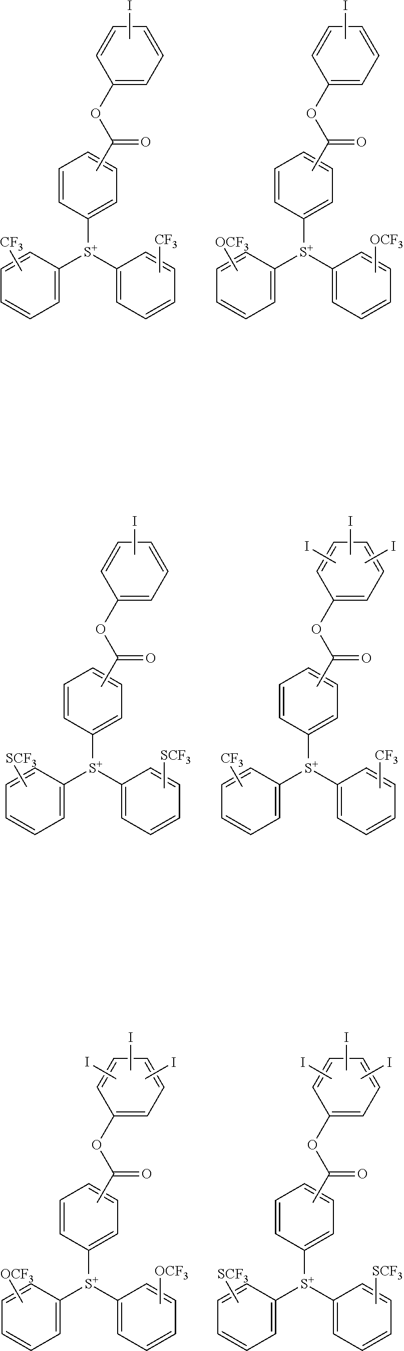

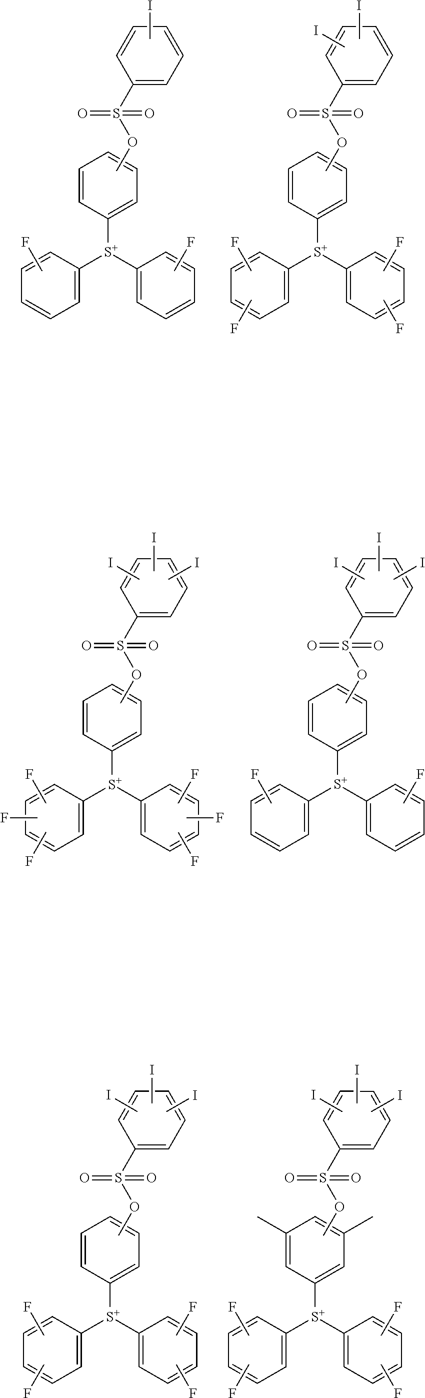

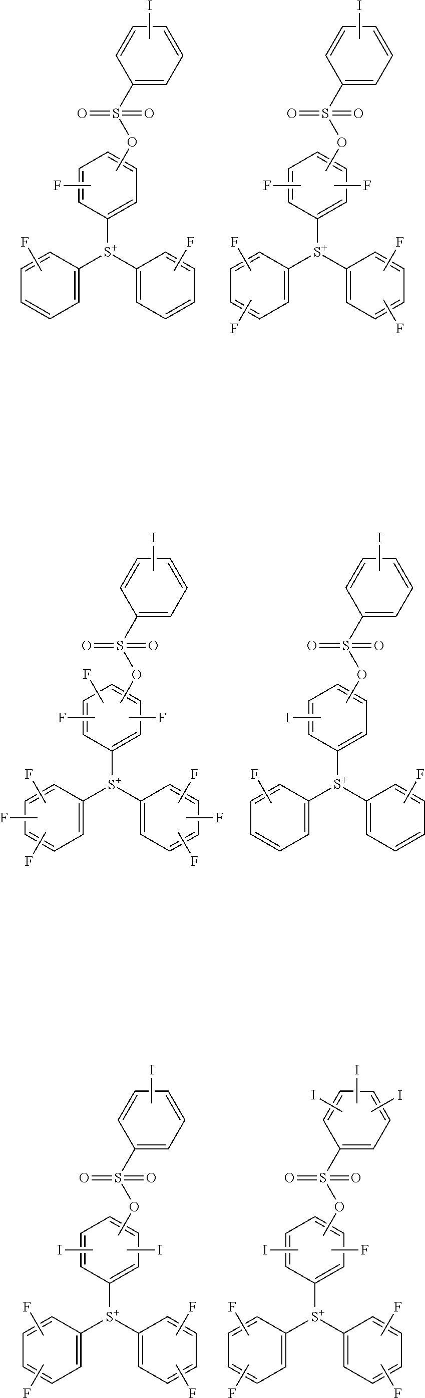

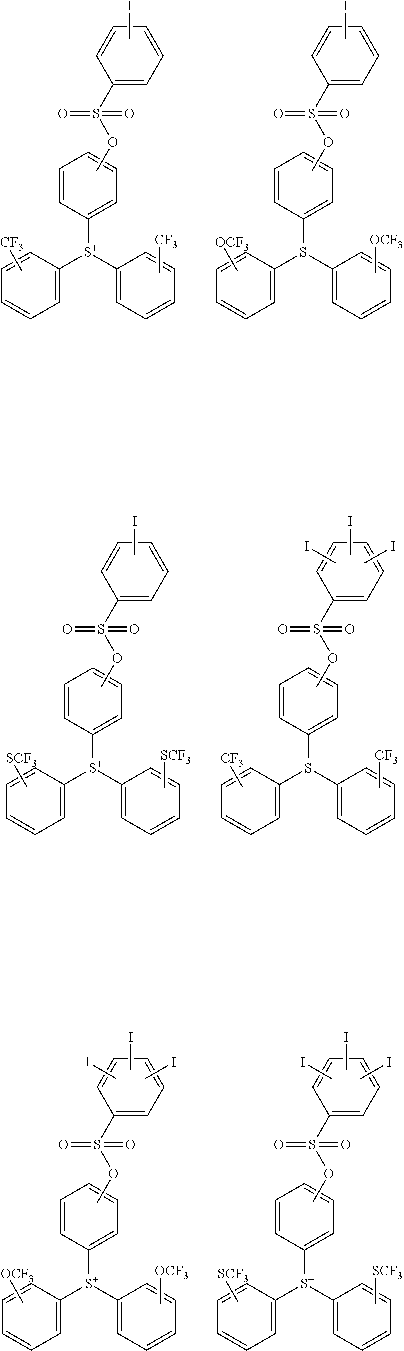

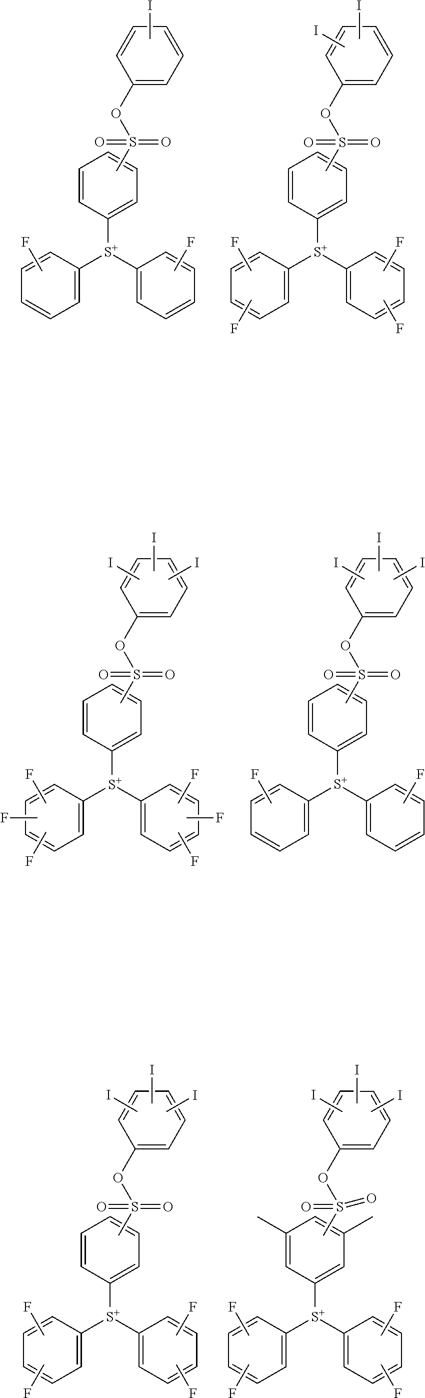

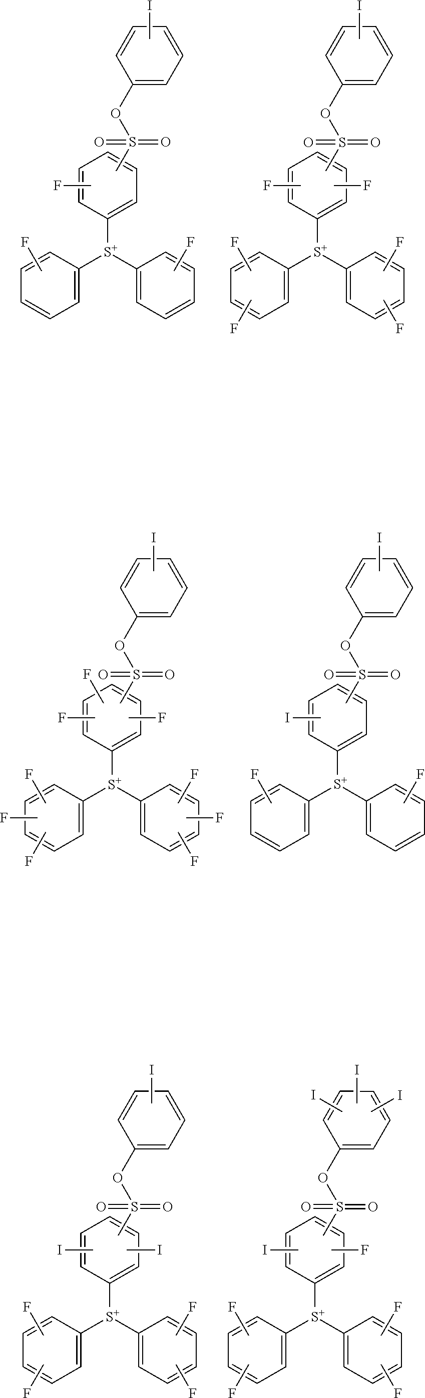

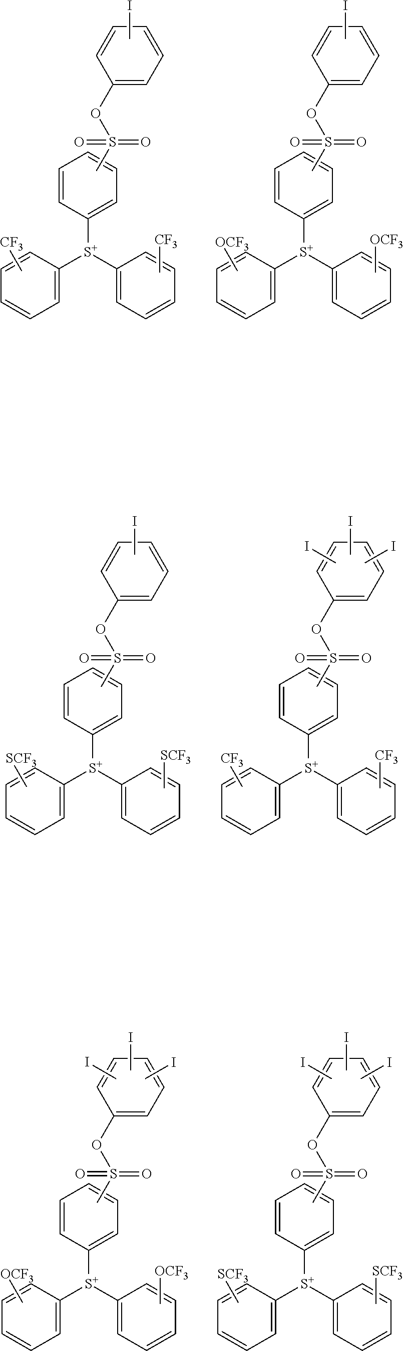

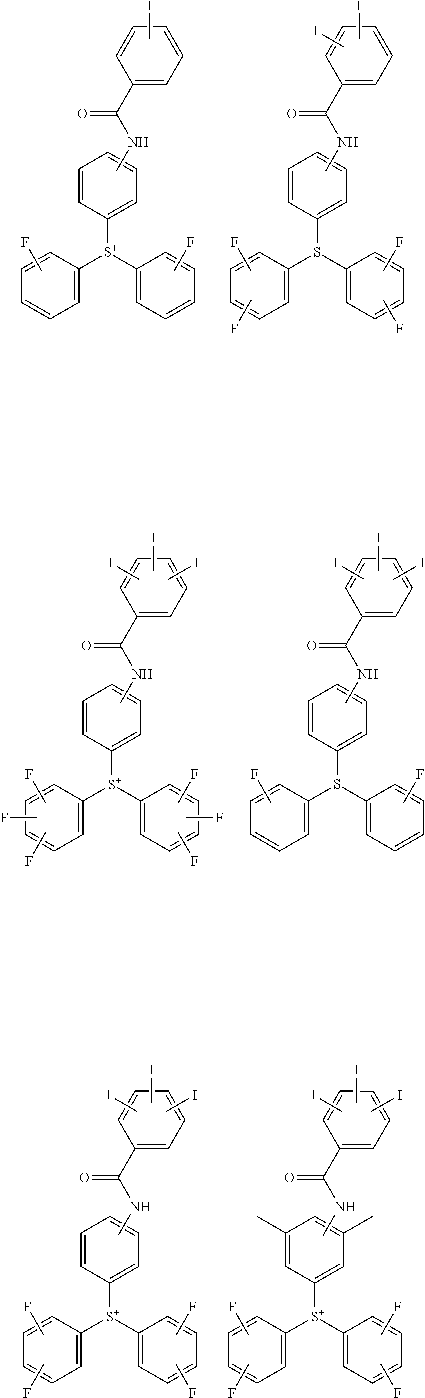

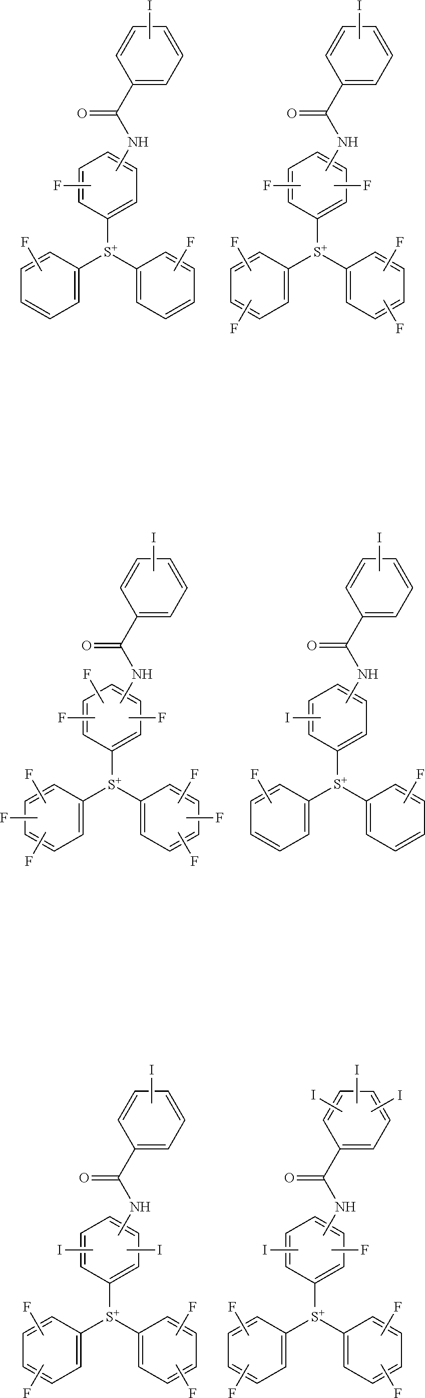

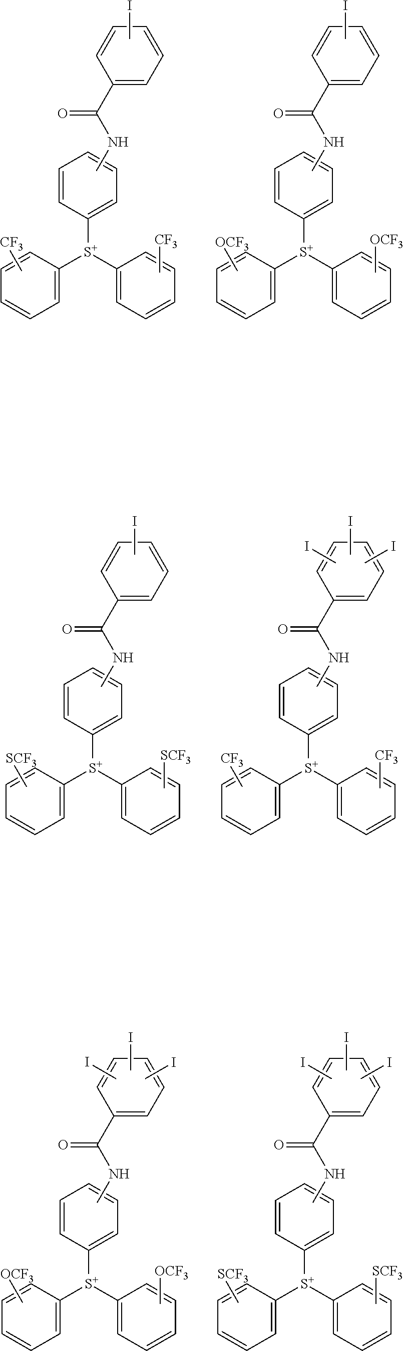

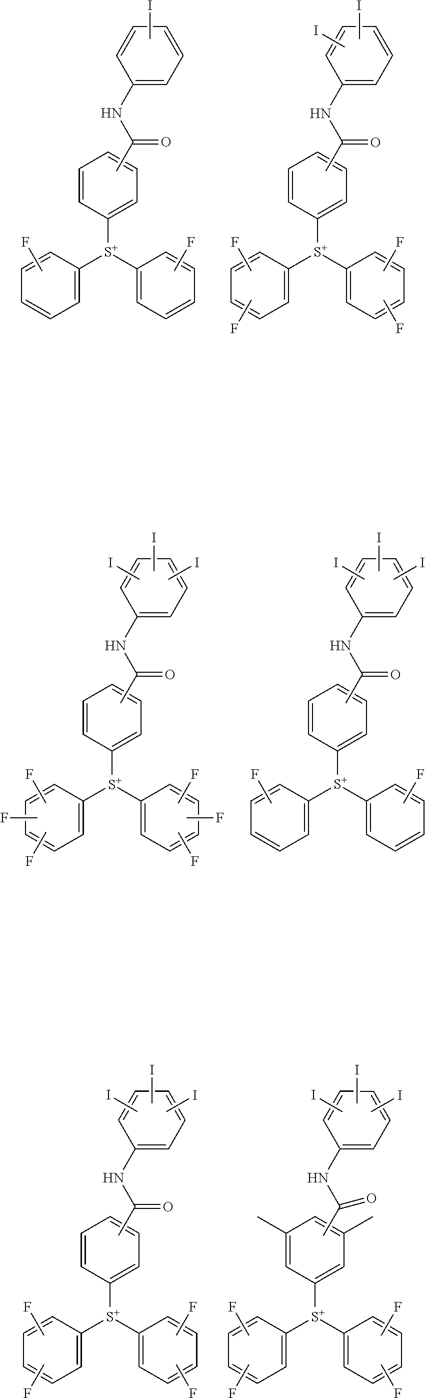

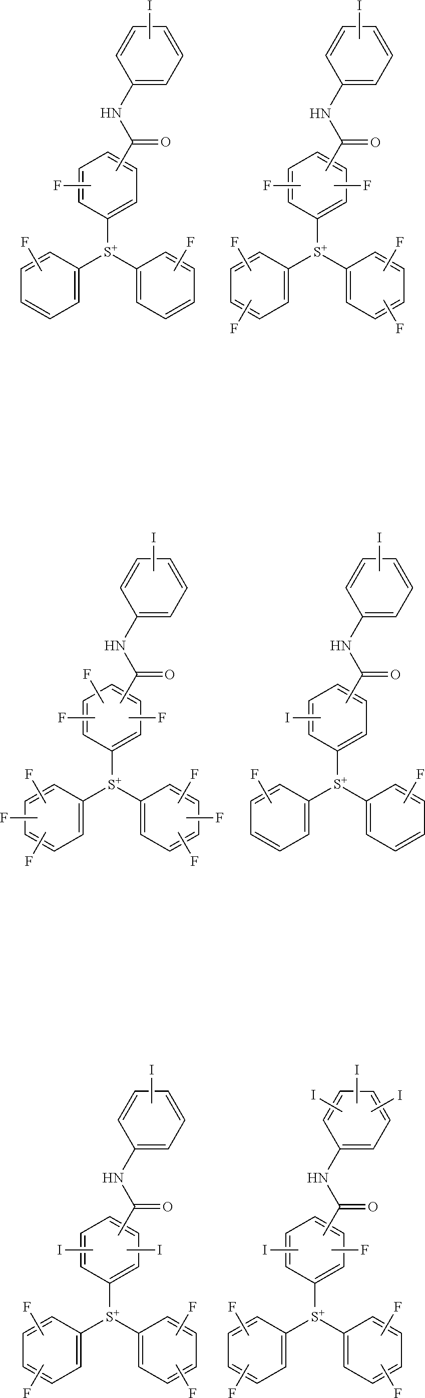

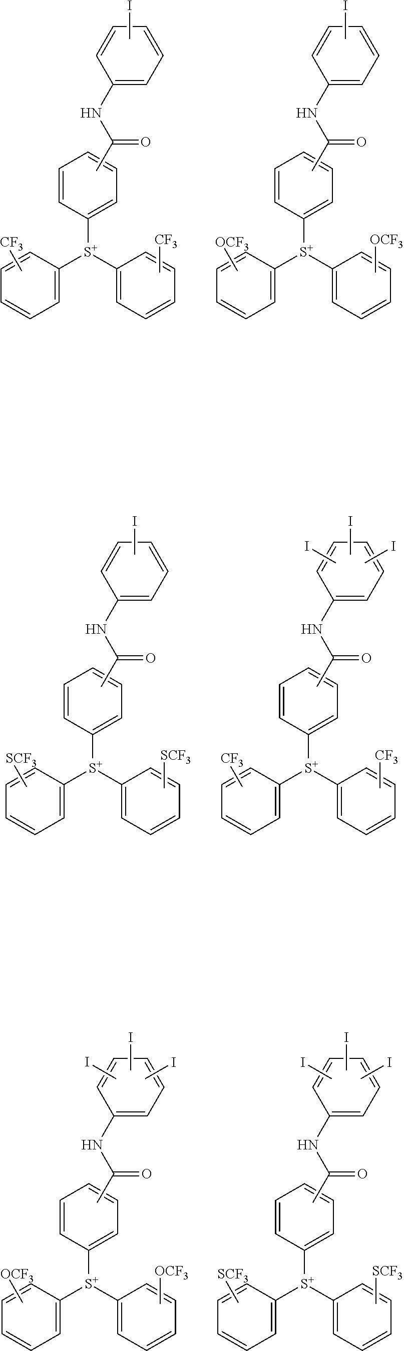

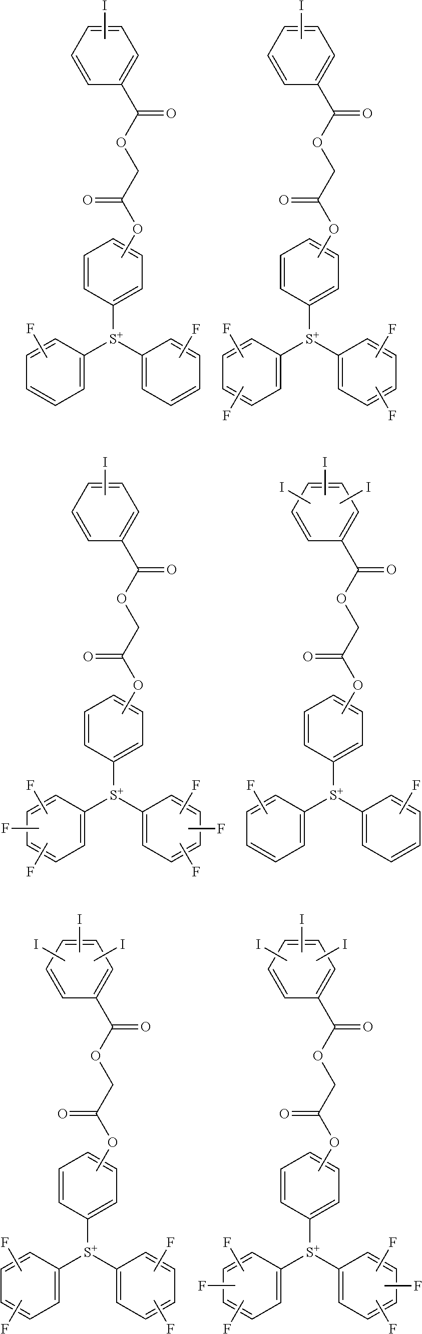

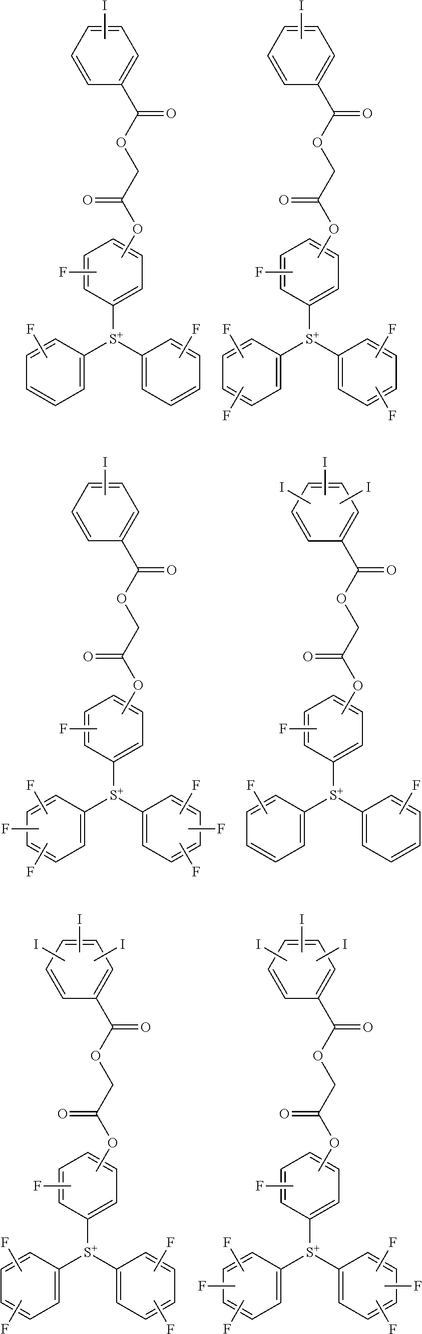

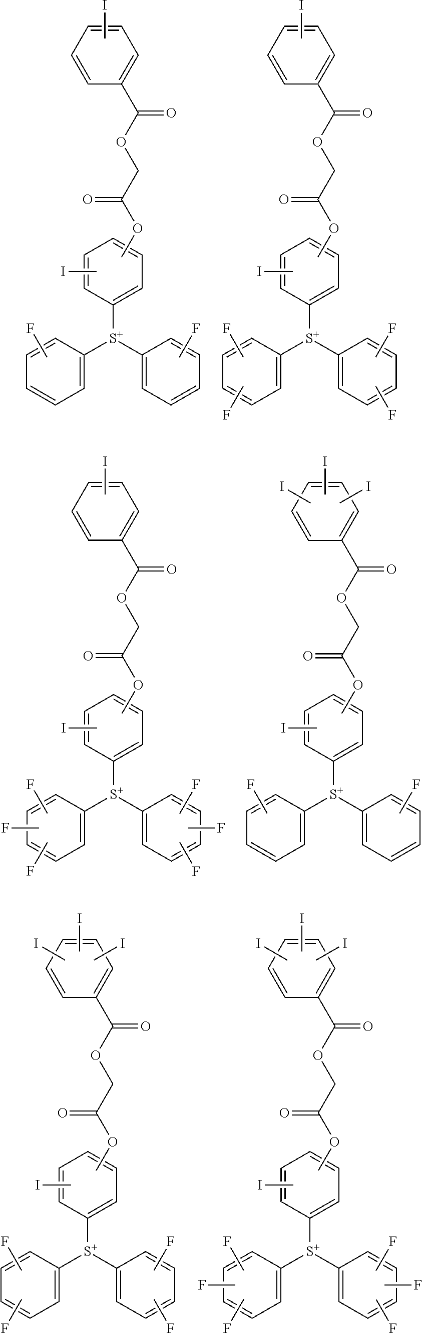

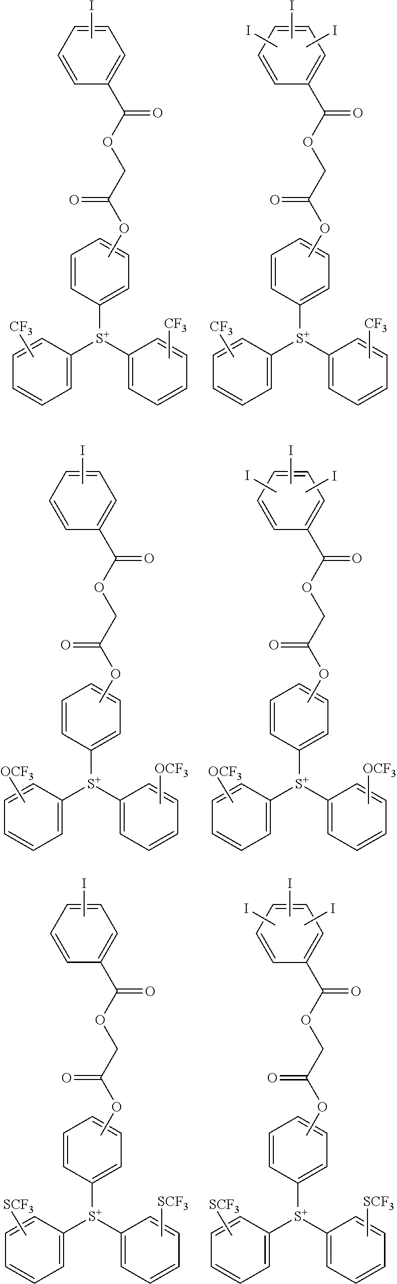

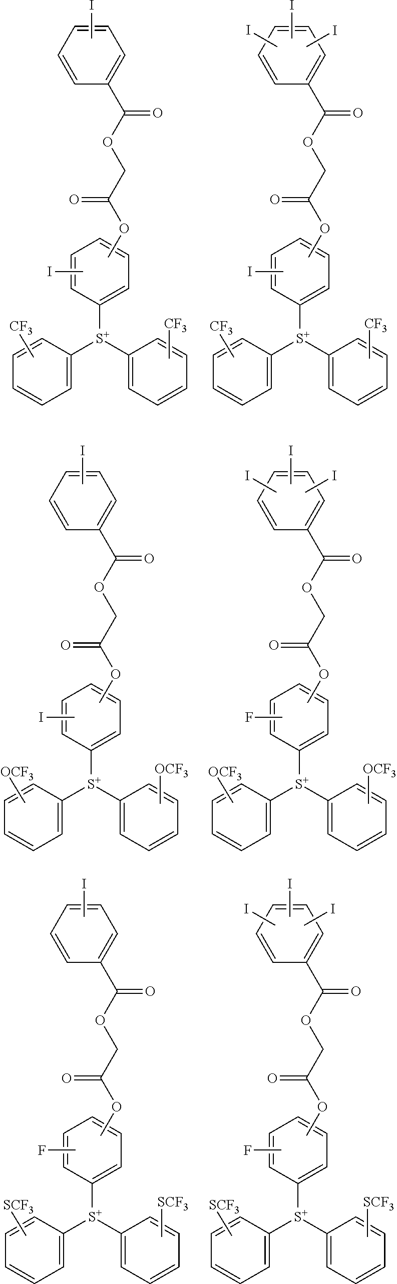

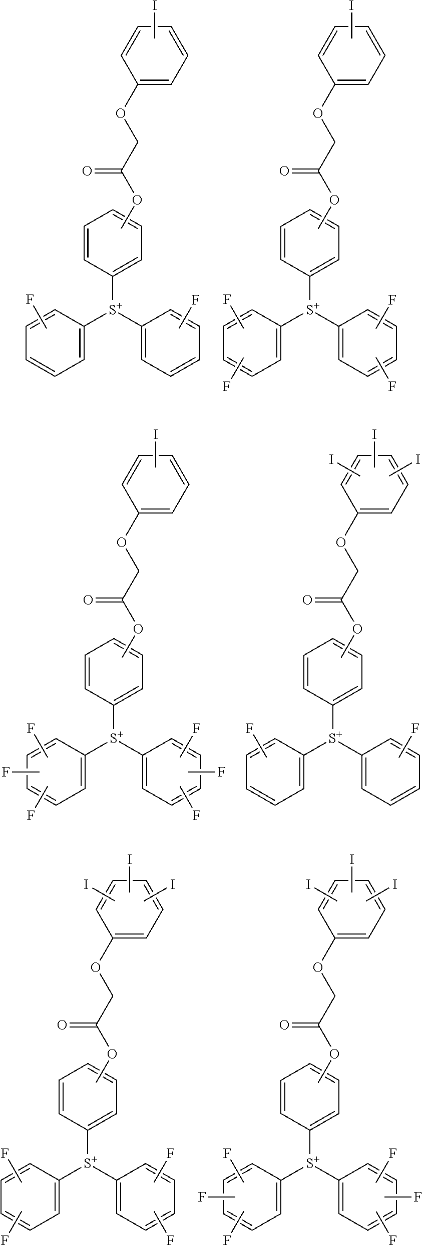

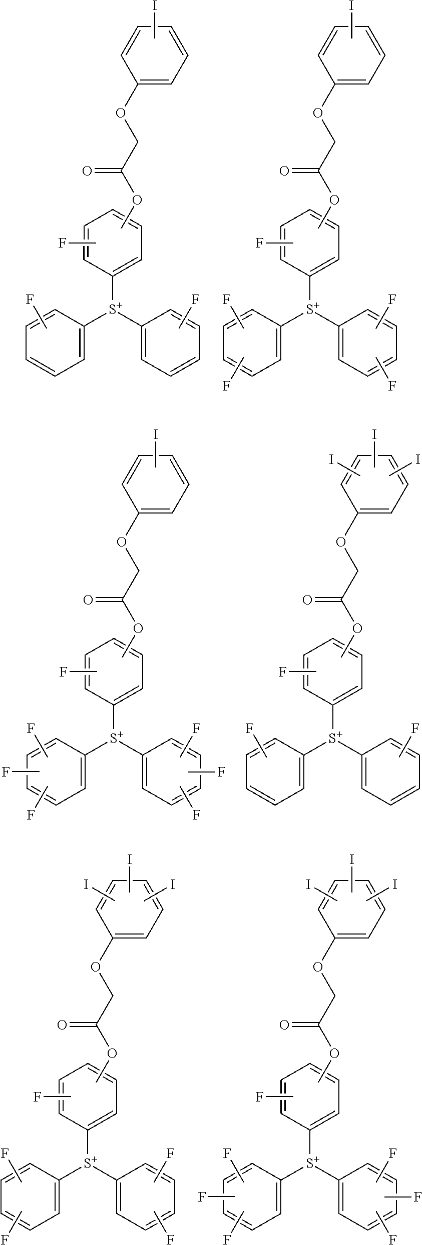

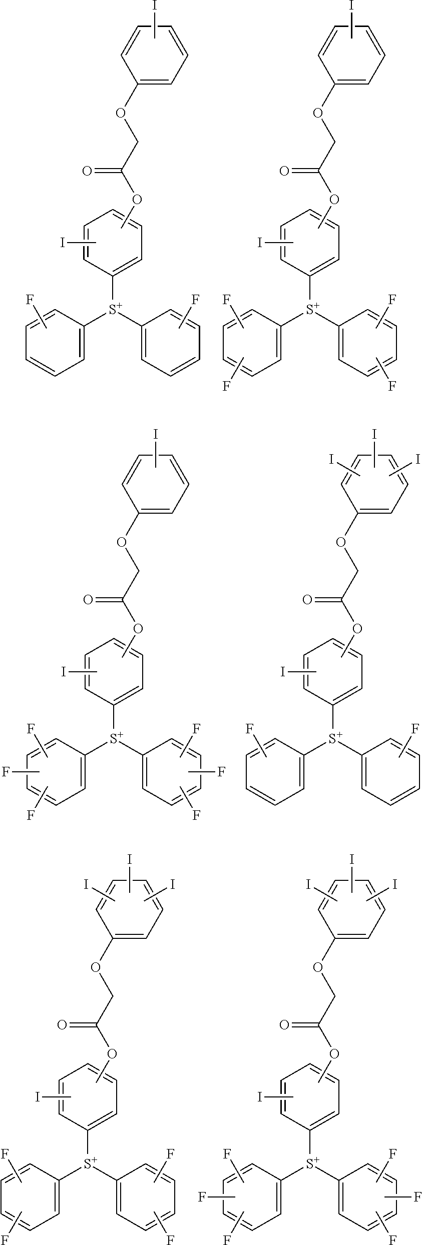

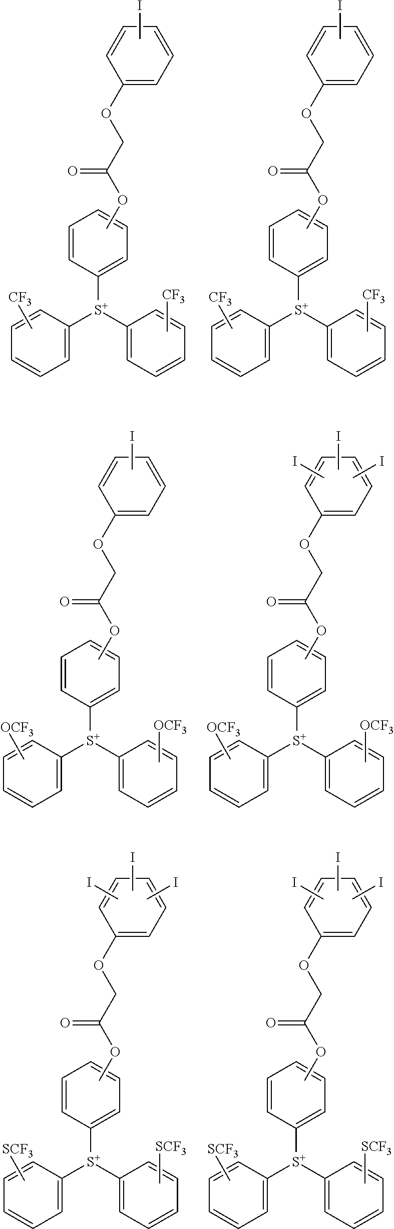

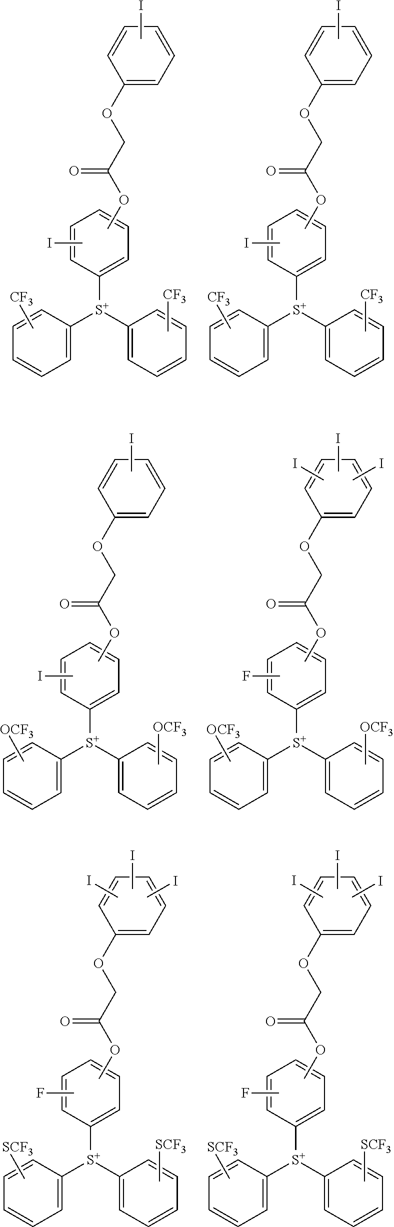

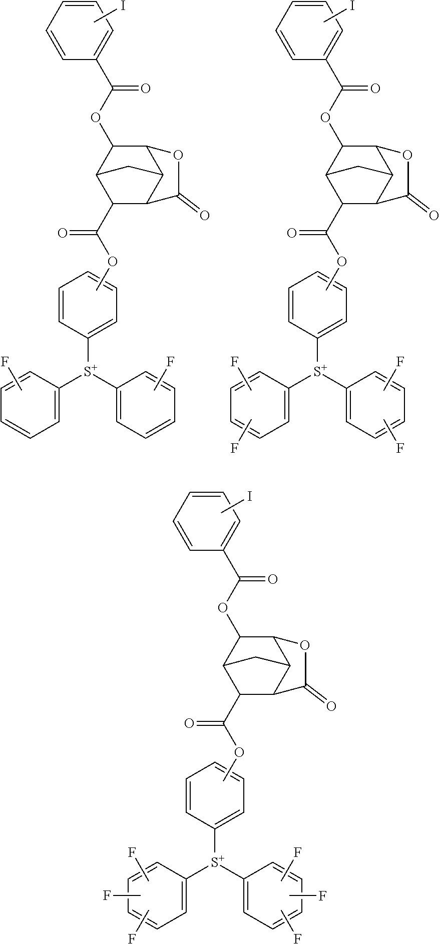

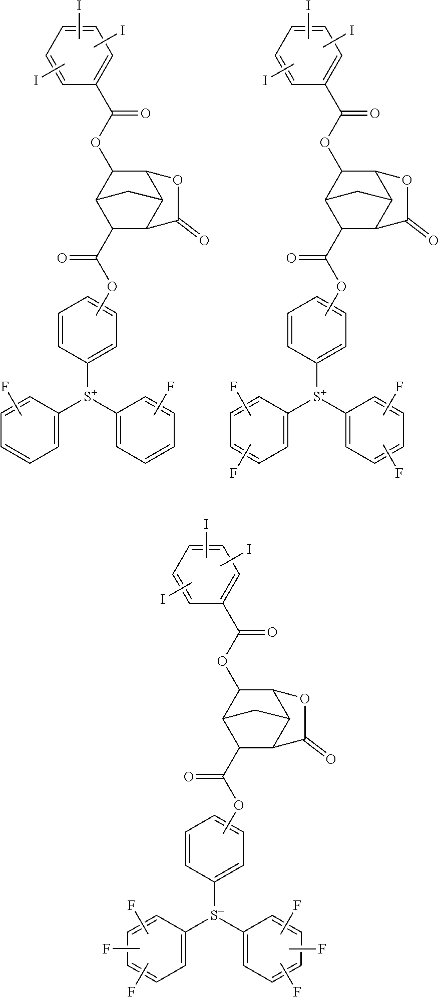

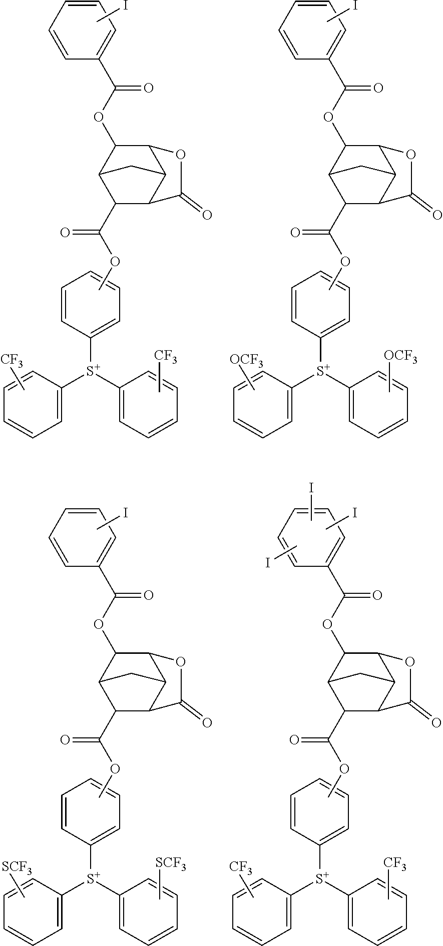

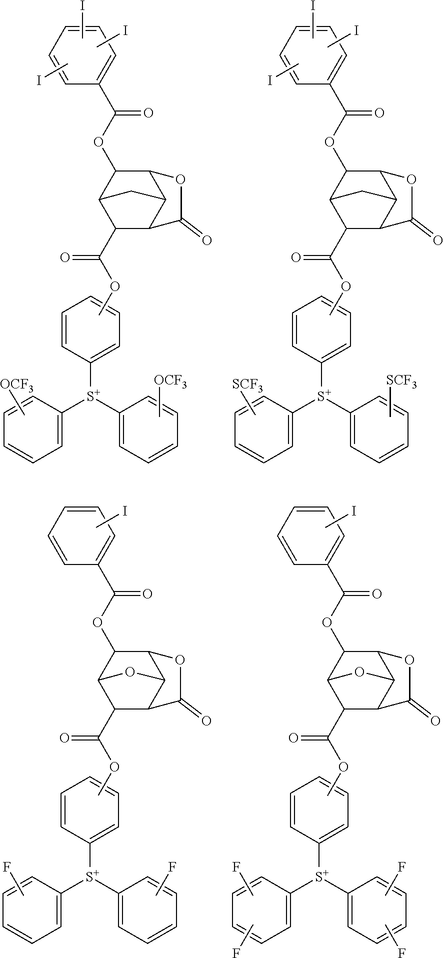

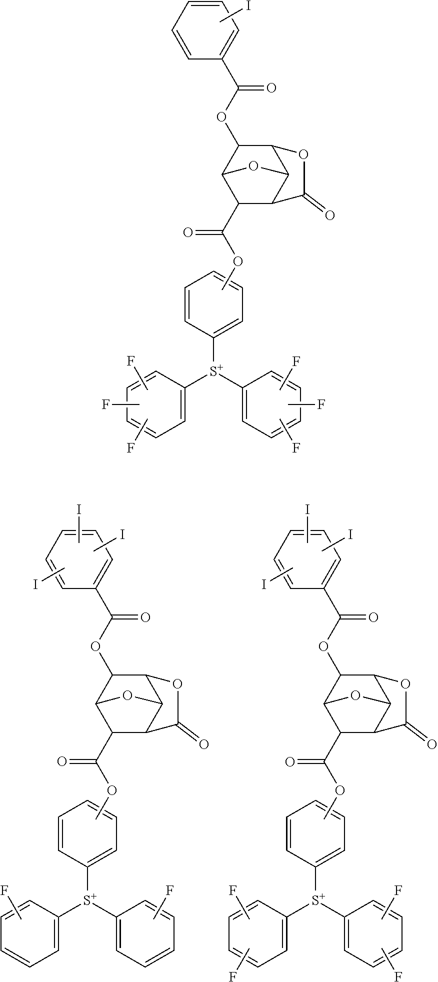

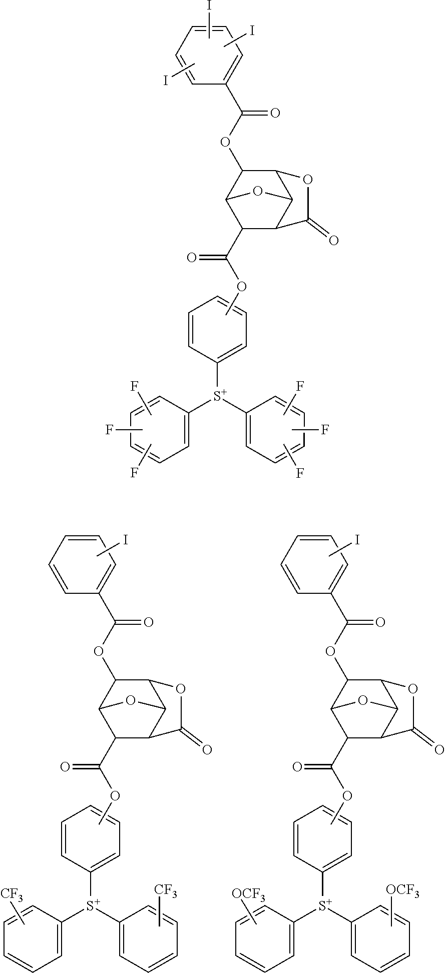

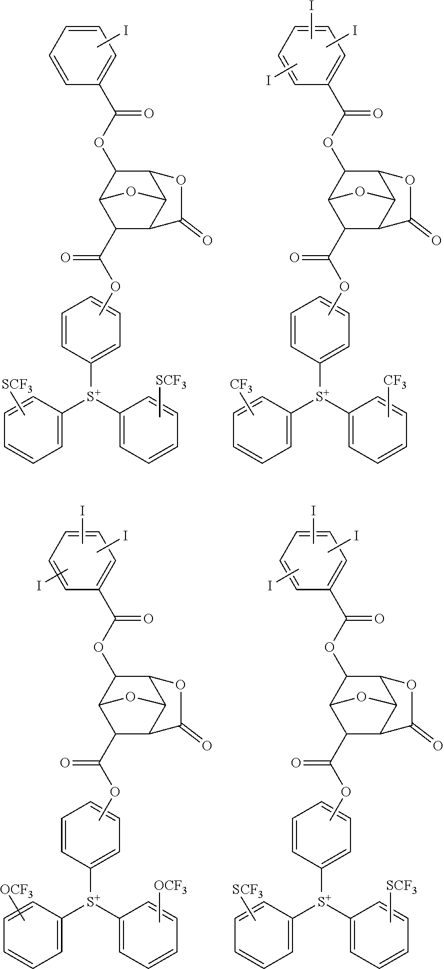

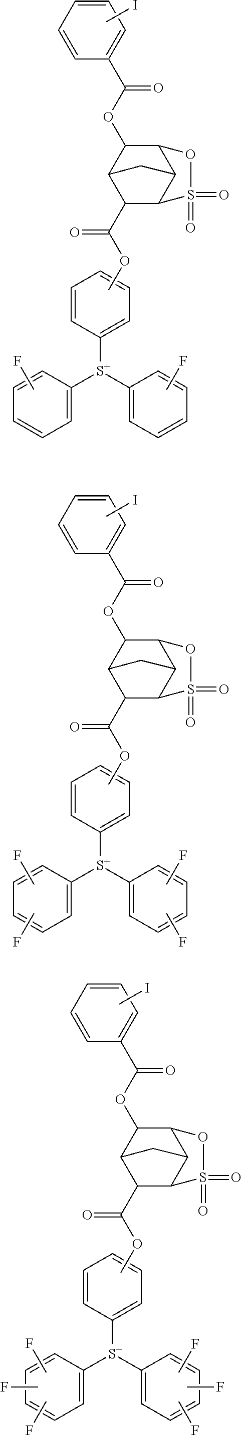

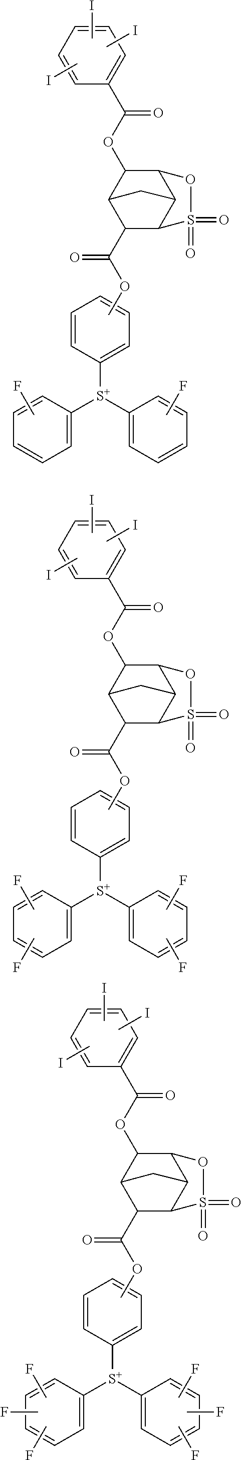

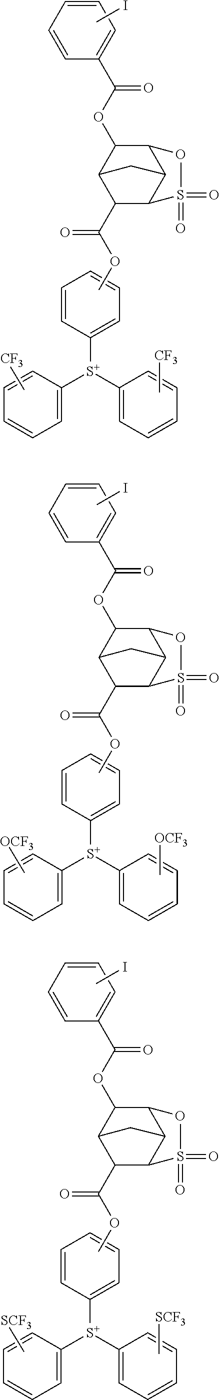

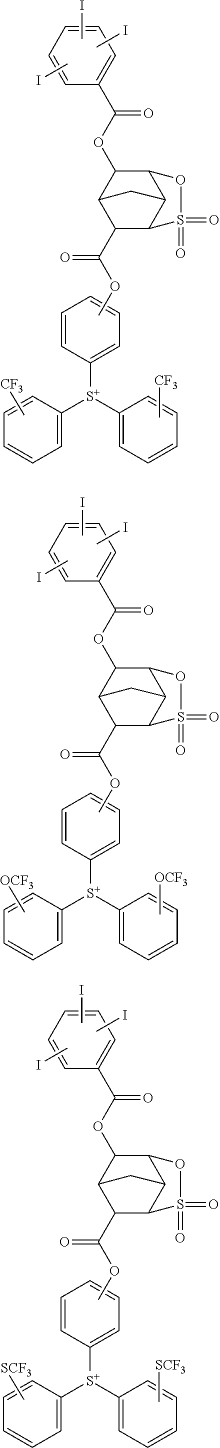

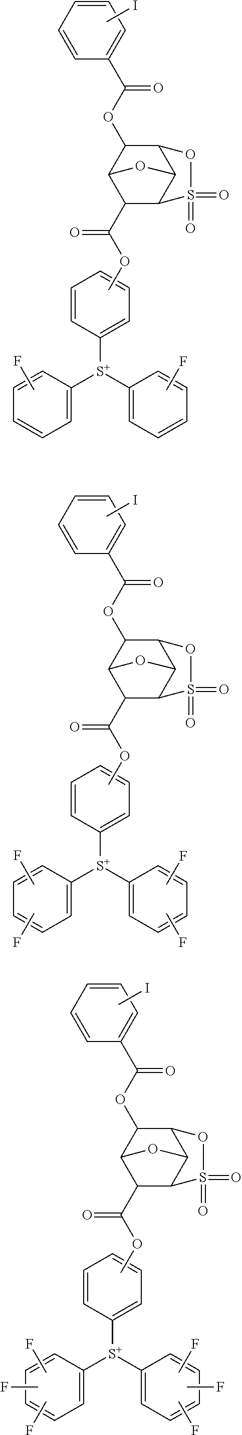

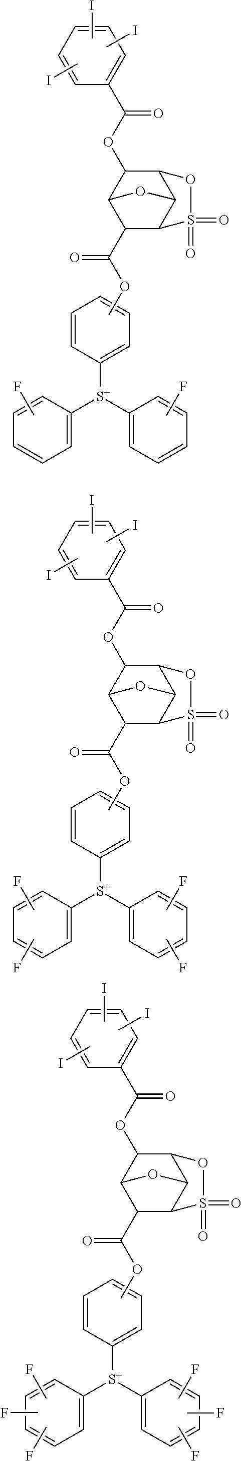

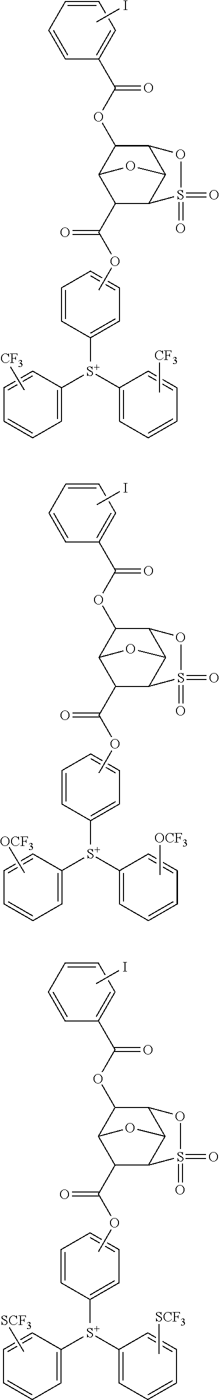

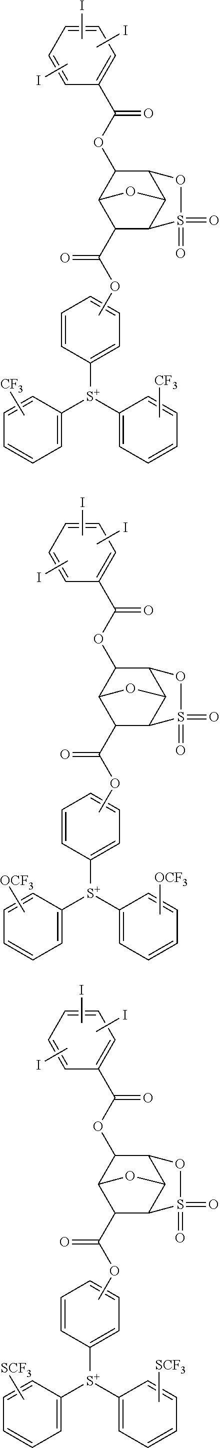

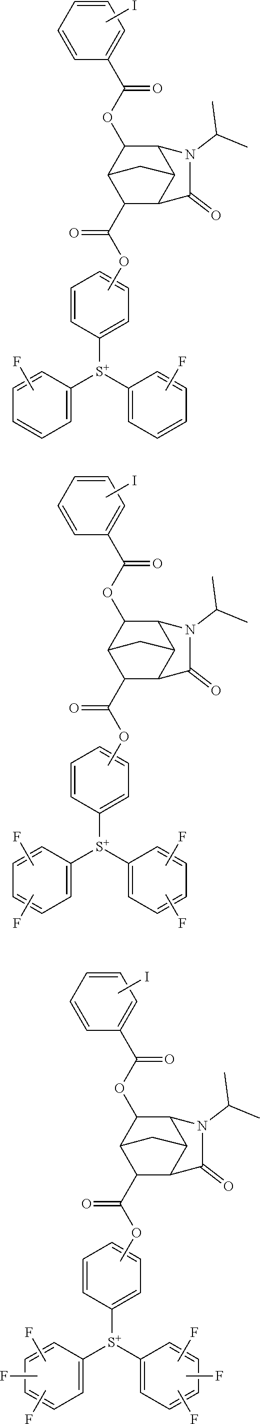

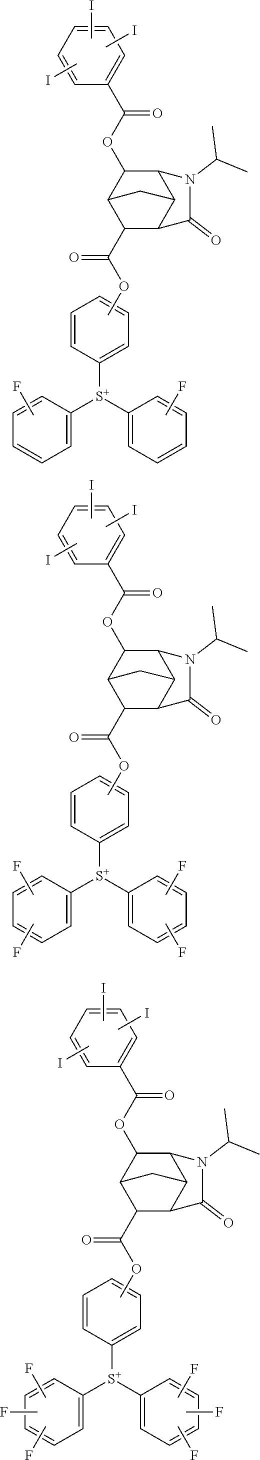

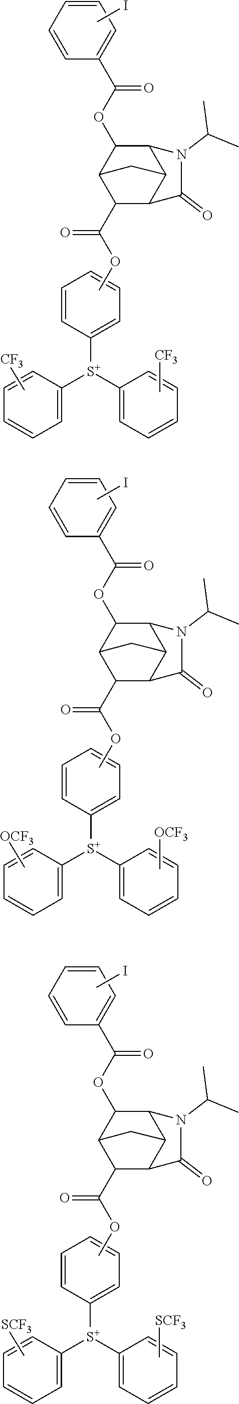

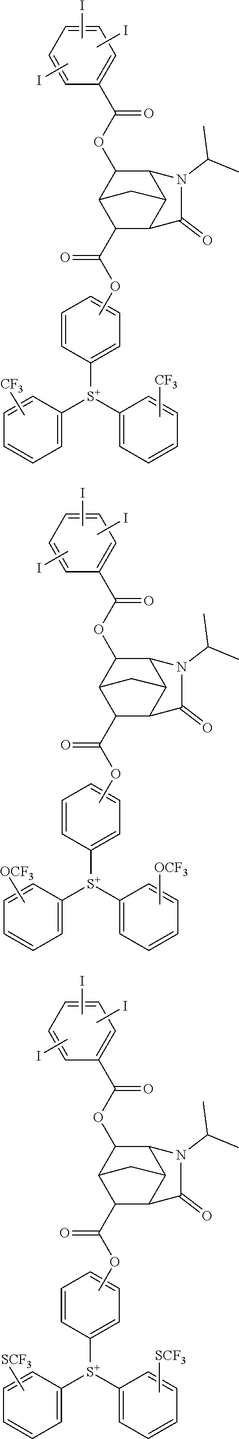

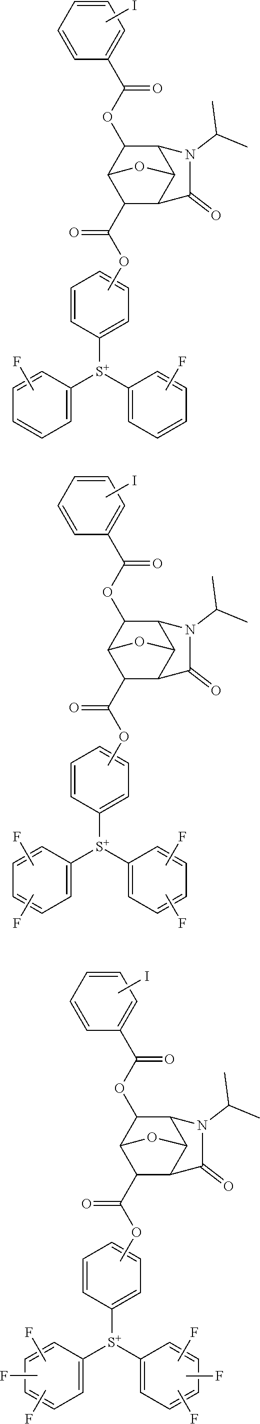

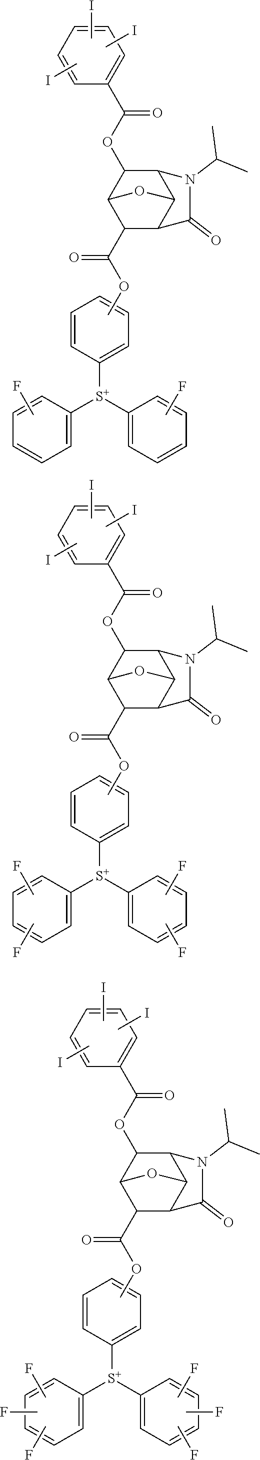

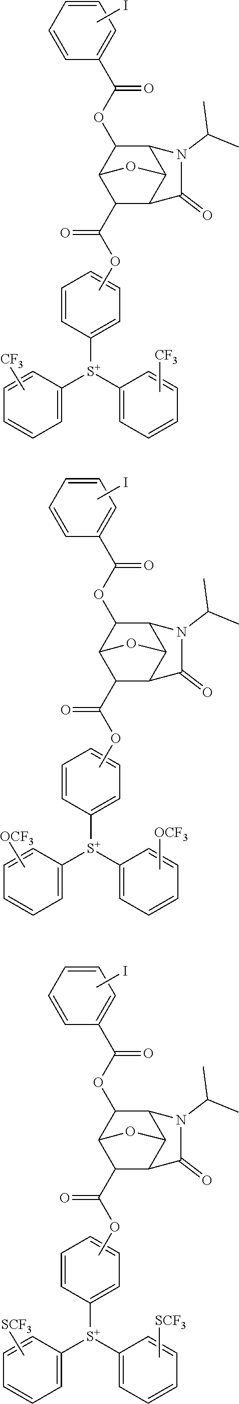

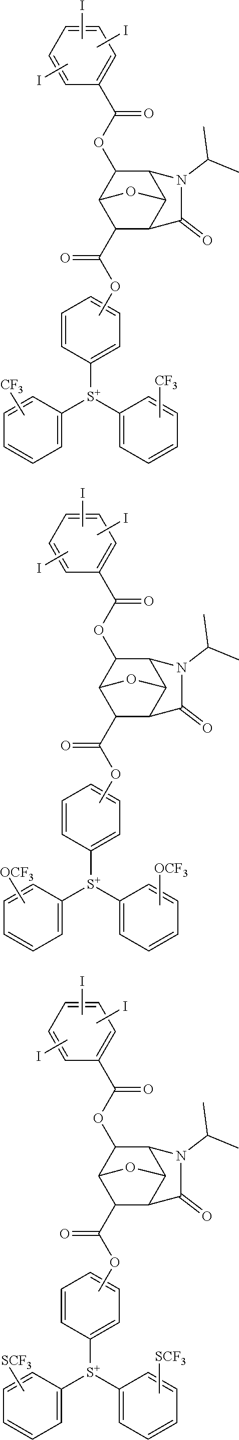

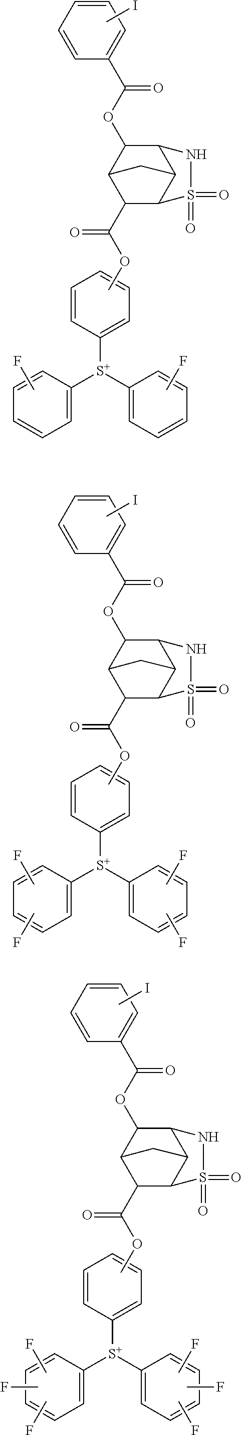

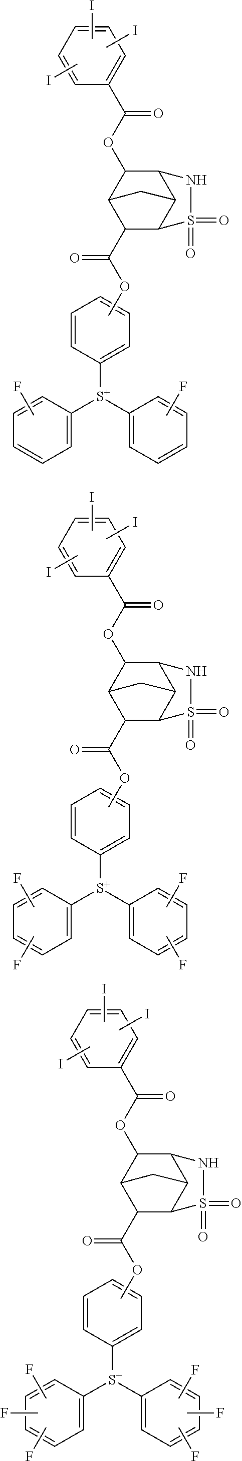

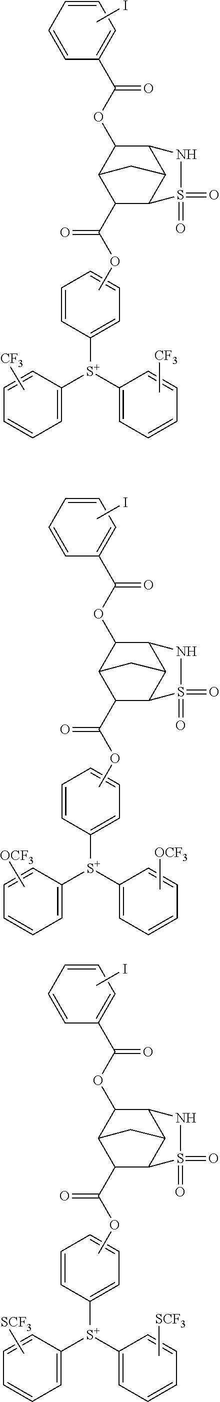

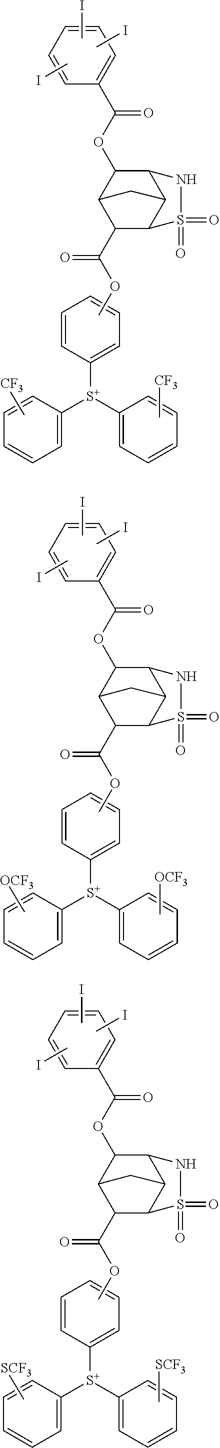

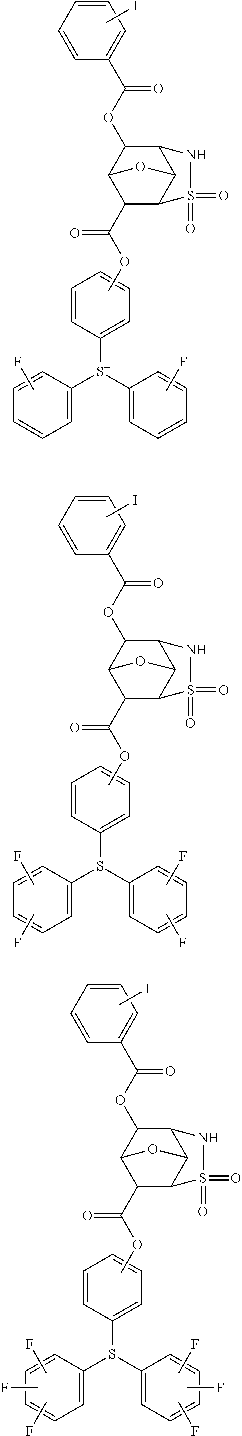

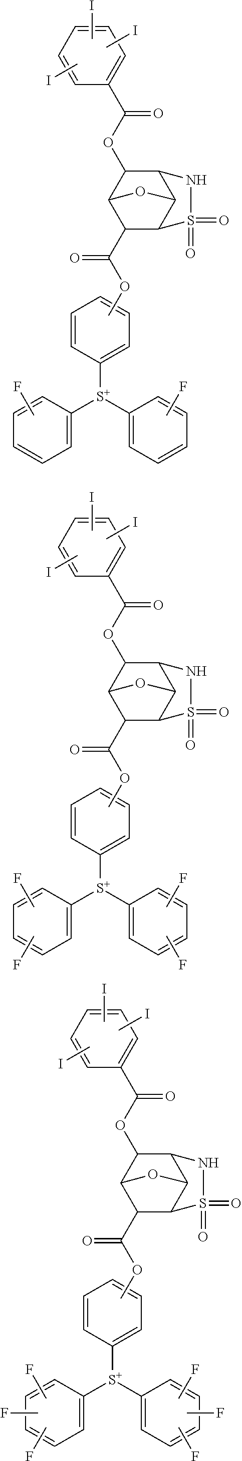

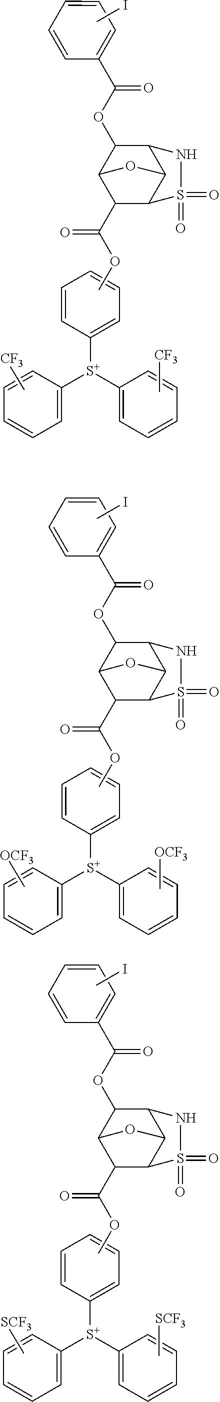

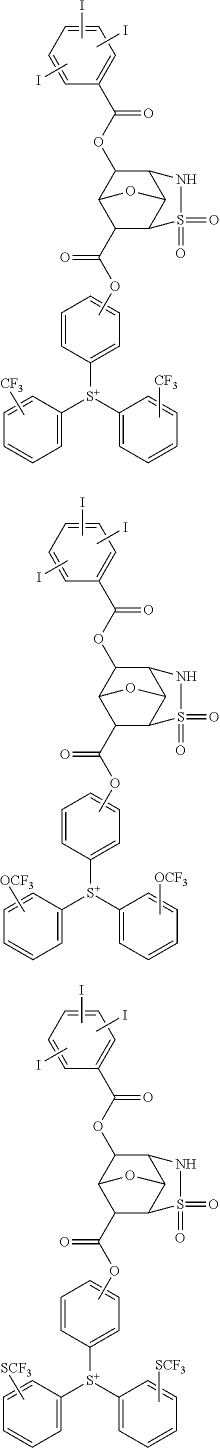

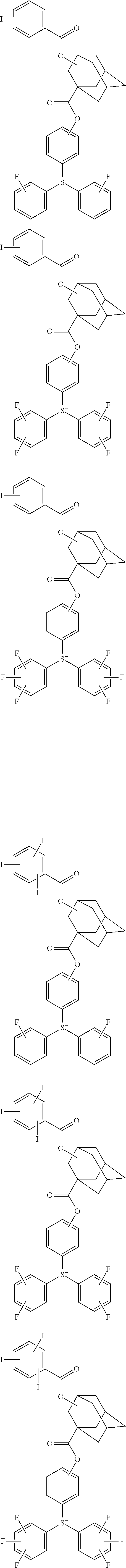

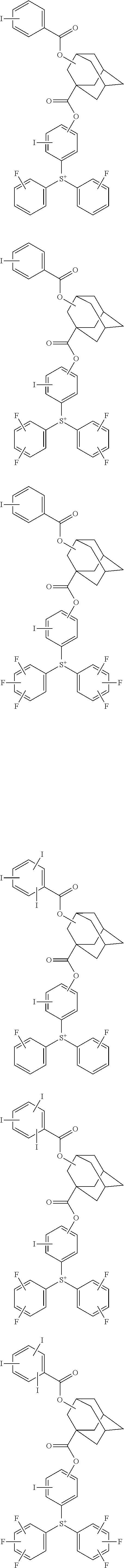

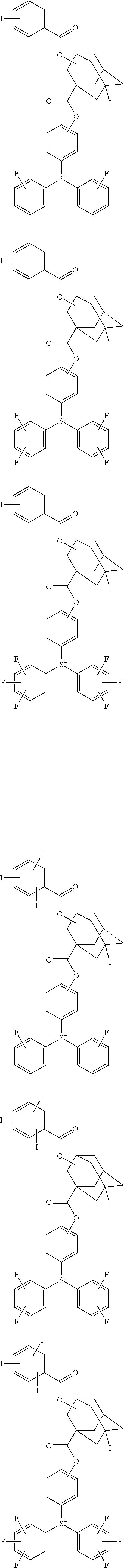

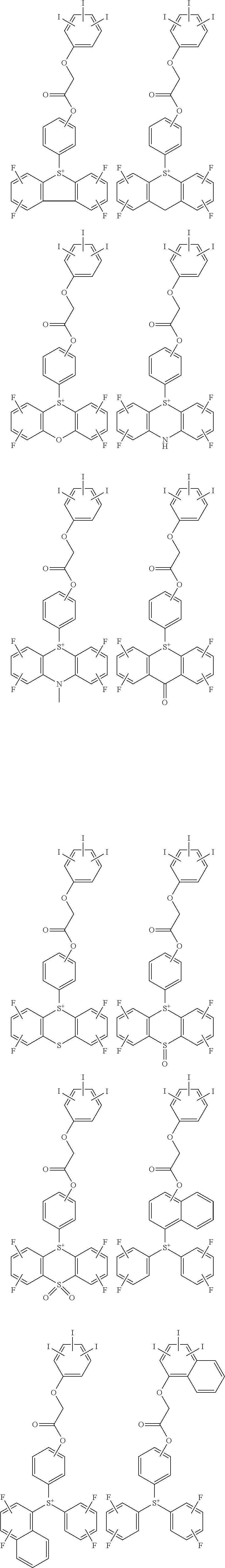

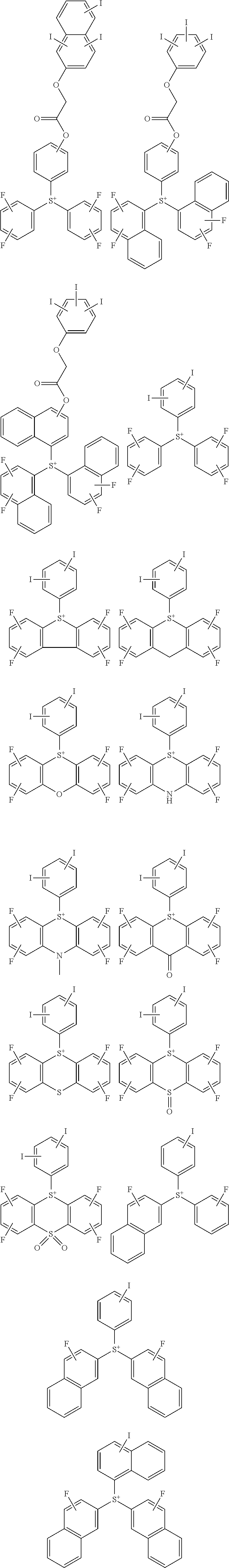

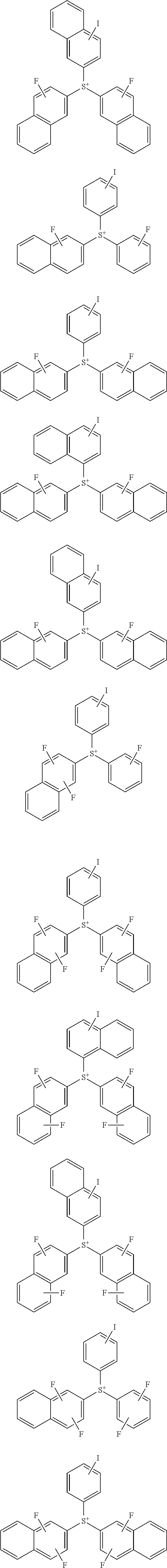

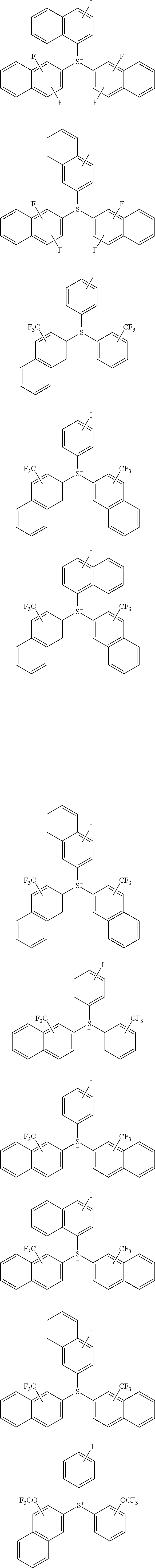

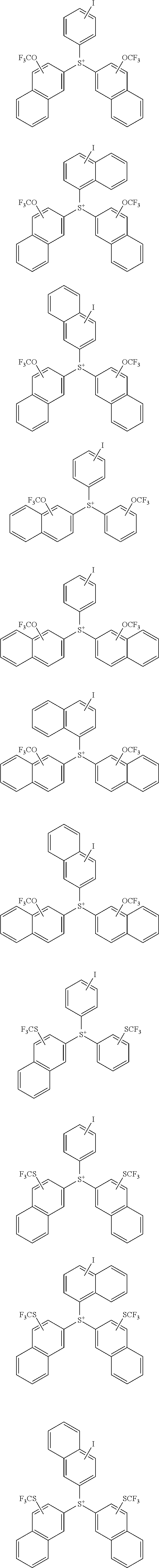

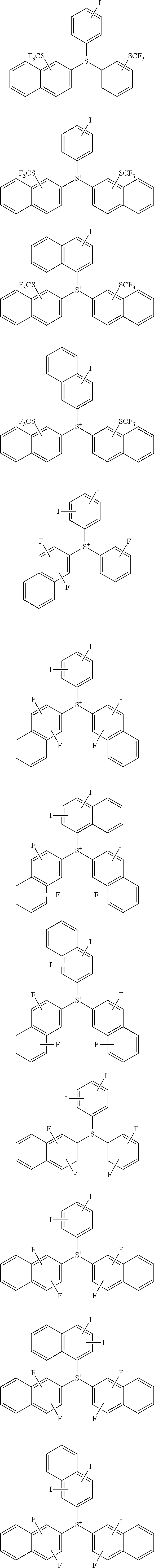

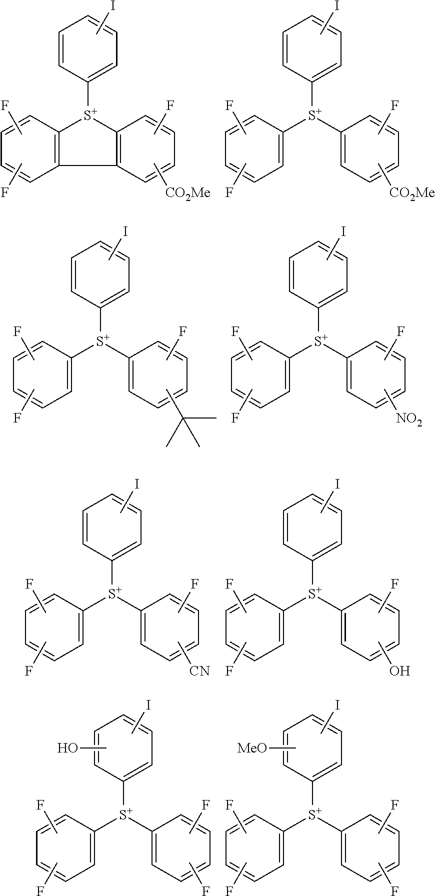

Examples of the iodonium cation include those described in JP-A 2024-000259, paragraph [0181], but not limited thereto.

Examples of the ammonium cation include ammonium cations having the formula (am-1).

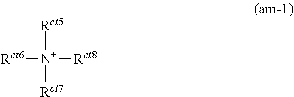

In the formula (am-1), R^{ct5} and R^{ct8} are each independently a C₁-C₄₀ hydrocarbyl group which may contain a heteroatom. R^{ct5} and R^{ct6} may bond together to form a ring with the nitrogen atom to which they are bonded. Examples of the hydrocarbyl group are as exemplified above for the hydrocarbyl group represented by R¹ in the formula (1A).

Examples of the ammonium cations having the formula (am-1) include those shown below, but not limited thereto.

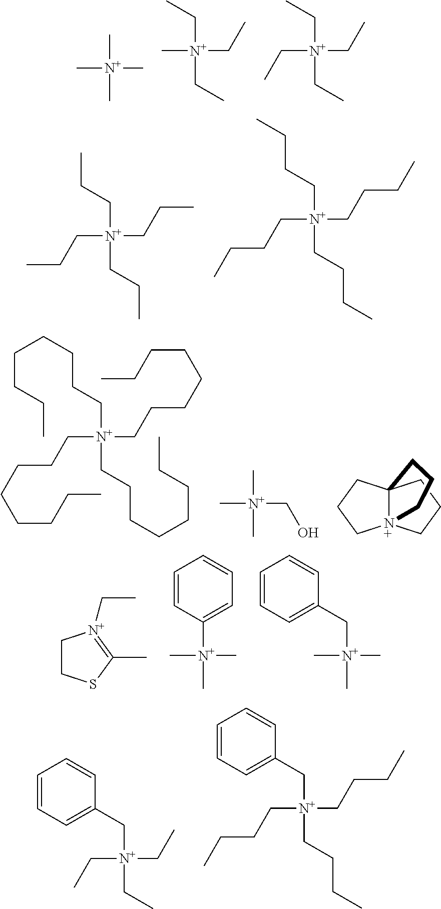

Exemplary structures of the repeat units (c1) to (c5) include arbitrary combinations of anions with cations, both as exemplified above.

Of the repeat units (c1) to (c5), repeat units (c2) to (c5) are preferred from the aspect of controlling acid diffusion. Repeat units (c2), (c4) and (c5) are more preferred from the aspect of the acid strength of generated acid. Repeat units (c2) are most preferred from the aspect of solvent solubility.

The base polymer may comprise repeat units of a structure having a hydroxy group protected with an acid labile group, which are also referred to as repeat units (d). The repeat unit (d) is not particularly limited as long as the unit includes one or more structures having a hydroxy group protected with a protective group such that the protective group is decomposed to generate the hydroxy group under the action of acid, but repeat units having the formula (d1) are preferred.

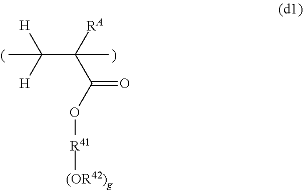

In the formula (d1), R^{A} is as defined above. R⁴¹ is a C₁-C₃₀ (g+1)-valent hydrocarbon group which may contain a heteroatom. R⁴² is an acid labile group. g is 1, 2, 3, or 4.

In the formula (d1), the acid labile group represented by R⁴² is deprotected under the action of acid so that a hydroxy group is generated. The structure of R⁴² is not particularly limited, but an acetal structure, ketal structure, alkoxycarbonyl group and alkoxymethyl group having the formula (d2) are preferred, with the alkoxymethyl group having the formula (d2) being most preferred.

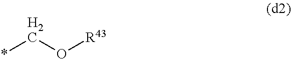

Herein the asterisk (*) denotes a valence bond, and R⁴³ is a C₁-C₁₅ hydrocarbyl group.

Examples of the acid labile group represented by R⁴², the alkoxymethyl group having the formula (d2), and the repeat units (d) are as exemplified for the repeat units (d) in JP-A 2020-111564 (US 20200223796).

The base polymer may contain repeat units (e) derived from indene, benzofuran, benzothiophene, acenaphthylene, chromone, coumarin, norbornadiene, or a derivative thereof. Examples of the monomer from which the repeat units (e) are derived include those shown below, but not limited thereto.

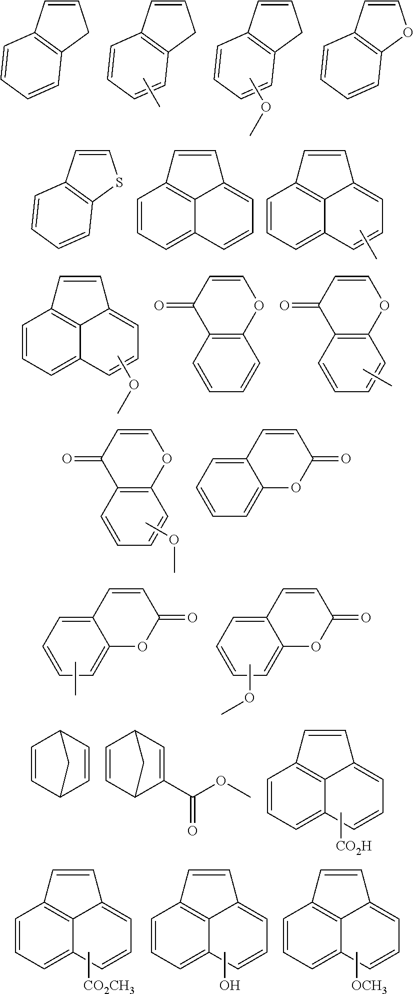

The base polymer may comprise repeat units (f) derived from indane, vinylpyridine or vinylcarbazole.

In the polymer, repeat units (a1), (a2), (a3), (bl), (b2), (c1), (c2), (c3), (c4), (d), (e), and (f) are incorporated in a ratio of preferably 0≤a1≤0.8, 0≤a2≤0.8, 0≤a3≤0.6, 0≤b1≤0.8, 0≤b2≤0.5, 0≤c1≤0.4, 0≤c2≤0.4, 0≤c3≤0.4, 0≤c4≤0.4, 0≤d≤0.3, 0≤e≤0.3, and 0≤f≤0.3; more preferably 0≤a1≤0.7, 0≤a2≤0.7, 0≤a3≤0.5, 0≤b1≤0.7, 0≤b2≤0.4, 0≤c1≤0.3, 0≤c2≤0.3, 0≤c3≤0.3, 0≤c4≤0.3, 0≤d≤0.2, 0≤e≤0.2, and 0≤f≤0.2. It is noted that a1+a2+a3+b1+b2+c1+c2+c3+c4+d+e+f≤1.0.

The polymer should preferably have a weight average molecular weight (Mw) in the range of 1,000 to 500,000, more preferably 3,000 to 100,000. A Mw in the range ensures that the resist film has sufficient etch resistance and eliminates the risk of resolution decline by a failure to provide a difference in dissolution rate before and after exposure. In the invention, Mw is measured by gel permeation chromatography (GPC) versus polystyrene standards using THF or N,N-dimethylformamide (DMF) solvent.

The influence of the molecular weight distribution (Mw/Mn) of the polymer becomes stronger as the pattern rule becomes finer. Therefore, the polymer should preferably have a narrow dispersity (Mw/Mn) of 1.0 to 2.0 in order to provide a resist composition suitable for micropatterning to a small feature size. A Mw/Mn in the range ensures that the contents of lower and higher molecular weight polymer fractions are low and eliminates a possibility that foreign matter is left on the pattern or the pattern profile is degraded.

The polymer may be synthesized, for example, by dissolving a monomer or monomers corresponding to the above-mentioned repeat units in an organic solvent, adding a radical polymerization initiator, and heating for polymerization.

Examples of the organic solvent which can be used in the polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of initiator added is preferably 0.01 to 25 mol % based on the total of monomers to be polymerized. The reaction temperature is preferably 50 to 150° C., more preferably 60 to 100° C. The reaction time is preferably 2 to 24 hours, more preferably 2 to 12 hours from the aspect of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution separately to the reactor. Because of a possibility that in the standby duration, the initiator generates a radical which triggers polymerization reaction to form an ultra high-molecular-weight polymer, it is preferred from the aspect of quality control to prepare the monomer solution and the initiator solution separately and add them dropwise. The acid labile group that has been incorporated in the monomer may be kept as such, or polymerization may be followed by protection or partial protection. Any known chain transfer agent such as dodecyl mercaptan or 2-mercaptoethanol may be added for molecular weight control purpose. The amount of chain transfer agent added is preferably 0.01 to 20 mol % based on the total of monomers to be polymerized.

When a hydroxy-containing monomer is copolymerized, the hydroxy group may be substituted by an acetal moiety which is susceptible to deprotection with acid, typically ethoxyethoxy, prior to polymerization, and the polymerization may be followed by deprotection with weak acid and water. Alternatively, the hydroxy group may be substituted by an acetyl, formyl or pivaloyl moiety prior to polymerization, and the polymerization may be followed by alkaline hydrolysis.

When hydroxystyrene or hydroxyvinylnaphthalene is copolymerized, one method is dissolving hydroxystyrene or hydroxyvinylnaphthalene and other monomers in an organic solvent, adding a radical polymerization initiator thereto, and heating the solution for polymerization. In an alternative method, acetoxystyrene or acetoxyvinylnaphthalene is used instead, and after polymerization, the acetoxy group is deprotected by alkaline hydrolysis, for thereby converting the polymer product to polyhydroxystyrene or hydroxypolyvinylnaphthalene.

For alkaline hydrolysis, a base such as aqueous ammonia or triethylamine may be used. The reaction temperature is preferably −20 to 100° C., more preferably 0 to 60° C. The reaction time is preferably 0.2 to 100 hours, more preferably 0.5 to 20 hours.

The amounts of monomers in the monomer solution may be determined appropriate so as to provide the preferred fractions of repeat units.

It is now described how to use the polymer obtained by the above production method. The reaction solution resulting from polymerization reaction may be used as the final product. Alternatively, the polymer may be recovered in powder form through a purifying step such as re-precipitation step of adding the polymerization solution to a poor solvent and letting the polymer precipitate as powder, after which the polymer powder is used as the final product. It is preferred from the aspects of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145](U.S. Pat. No. 7,537,880). Exemplary solvents include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); high-boiling alcohols such as diethylene glycol, propylene glycol, glycerol, 1,4-butanediol, and 1,3-butanediol; and mixtures thereof.

The polymer solution preferably has a polymer concentration of 0.01 to 30 wt %, more preferably 0.1 to 20 wt %.

Prior to use, the reaction solution or polymer solution is preferably filtered through a filter. Filtration is effective for consistent quality because foreign matter and gel which can cause defects are removed.

Suitable materials of which the filter used for filtration is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer production process, the filtering step may be carried out any times, in any order and in any stage. The reaction solution as polymerized or the polymer solution may be filtered, preferably both are filtered.

The base polymer (B) may be used alone or as a combination of two or more polymers which differ in compositional ratio, Mw and/or Mw/Mn. The base polymer (B) may also comprise a hydrogenated ring-opened metathesis polymer in addition to the polymer. For the ring-opening metathesis polymer, one described in JP-A 2003-066612 can be used.

### (C) Organic Solvent

The chemically amplified resist composition of the invention may comprise an organic solvent as component (C). The organic solvent (C) is not particularly limited as long as the components described above and below are soluble therein. Examples of the organic solvent include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; and mixtures thereof.

Of the foregoing organic solvents, 1-ethoxy-2-propanol, PGMEA, cyclohexanone, GBL, DAA and mixtures thereof are preferred because the base polymer (B) is most soluble therein.

The amount of the organic solvent (C) in the chemically amplified resist composition of the invention is preferably 200 to 5,000 parts by weight, more preferably 400 to 3,500 parts by weight per 80 parts by weight of the base polymer (B). The organic solvent (C) may be used alone or in admixture of two or more.

### (D) Photoacid Generator

The chemically amplified resist composition of the invention may comprise a photoacid generator as component (D). The photoacid generator is not particularly limited as long as it is capable of generating an acid having a higher acid strength than the sulfonic acid generated by the quencher (A), upon exposure to high-energy radiation.

Preferred examples of the photoacid generator include those having the formula (2) or (3).

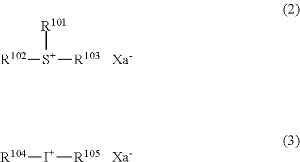

In the formula (2), R¹⁰¹ to R¹⁰⁵ are each independently a C₁-C₂₀ hydrocarbyl group which may contain a heteroatom. Any two of R¹⁰¹, R¹⁰² and R¹⁰³ may bond together to form a ring with the sulfur atoms to which they are attached. Examples of the hydrocarbyl group are as exemplified above for the hydrocarbyl group represented by R¹ in the formula (1A).

Examples of the sulfonium cation in the salt having the formula (2) include those described as examples of the sulfonium cation in the formula (1A), those described in JP-A 2024-003744, paragraphs [0102] to [0125], those described in WO 2024/128017, paragraphs [0044] to [0049], and those described in JP 7491173, paragraphs [0035] to [0046], and those described as examples of the sulfonium cation in the formula (d3), but not limited thereto. Examples of the iodonium cation in the salt having the formula (3) include those described in JP-A 2024-000259, paragraph [0181], but not limited thereto.

In the formulae (2) and (3), Xa is an anion of strong acid. Examples of the anion of strong acid include anions having any of the formulae (c1-1) to (c1-5).

Compounds having the formula (4) are also preferred as the photoacid generator (D).

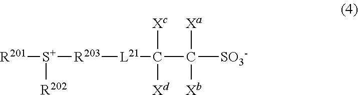

In the formula (4), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atoms to which they are attached.

The C₁-C₃₀ hydrocarbyl groups represented by R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl, and adamantyl; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl; and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

The C₁-C₃₀ hydrocarbylene group represented by R²⁰³ may be saturated or unsaturated and straight, branched or cyclic. Examples thereof include C1-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl, and adamantanediyl; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene. In the hydrocarbylene group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. Of the heteroatoms, oxygen is preferred.

In the formula (4), L²¹ is a single bond, ether bond or a C1-C₂₀ hydrocarbylene group which may contain a heteroatom. The hydrocarbylene group may be saturated or unsaturated and straight, branched or cyclic. Examples thereof include those exemplified above for the hydrocarbylene group represented by R²⁰³

In the formula (4), X^{a}, X^{b}, X^{c} and X^{d} are each independently hydrogen, fluorine or trifluoromethyl. at least one of X^{a}, X^{b}, X^{c} and X^{d} being fluorine or trifluoromethyl.

As the photoacid generator having the formula (4), a photoacid generator having the formula (4′) is preferred.

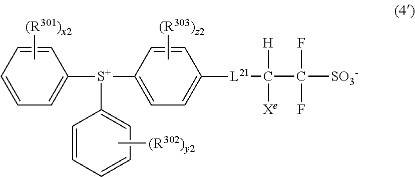

In the formula (4′), L²¹ is as defined above. X^{e} is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof include those exemplified above for the hydrocarbyl group represented by R^{fa1} in the formula (c1-1-1). x2 and y2 are each independently 0, 1, 2, 3, 4, or 5, and z2 is 0, 1, 2, 3, or 4.

Examples of the photoacid generator having the formula (4) include those exemplified for the photoacid generator having the formula (2) in JP-A 2017-026980.

Of the photoacid generators, those having an anion having the formula (c1-1-1) or (c1-4) are especially preferred because of reduced acid diffusion and high solubility in solvents. Also those having the formula (4′) are especially preferred because of extremely reduced acid diffusion.

When the chemically amplified resist composition of the invention comprises the photoacid generator (D), the amount thereof is preferably 0.1 to 40 parts by weight, more preferably 0.5 to 20 parts by weight per 80 parts by weight of the base polymer (B). As long as the amount of the photoacid generator (D) is in the range, good resolution is achievable and the risk of foreign matter being formed after development or during stripping of resist film is avoided. The photoacid generator (D) may be used alone or in combination of two or more.

### (E) Other Quencher

The chemically amplified resist composition of the present invention may further comprise (E) a quencher other than component (A), which is also referred to as other quencher.

Examples of the other quencher (E) include an onium salt having the formula (5) or (6).

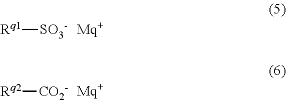

In the formula (5), R^{q1} is hydrogen or a C₁-C₄₀ hydrocarbyl group which may contain a heteroatom, exclusive of the group wherein hydrogen bonded to the carbon atom at α-position relative to the sulfo group is substituted by fluorine or fluoroalkyl. In the formula (6), R^{q2} is hydrogen or a C₁-C₄₀ hydrocarbyl group which may contain a heteroatom.

Examples of the C1-C₄₀ hydrocarbyl group represented by R^{q1} include C1-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, and adamantyl; C₆-C₄₀ aryl groups such as phenyl, naphthyl and anthracenyl. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

Examples of the hydrocarbyl group represented by R^{q2} include substituents exemplified above for R^{q1} as well as fluorinated saturated hydrocarbyl groups such as trifluoromethyl and trifluoroethyl, and fluorinated aryl groups such as pentafluorophenyl and 4-trifluoromethylphenyl.

Examples of the anion in the onium salt having the formula (5) include those shown below, but not limited thereto.

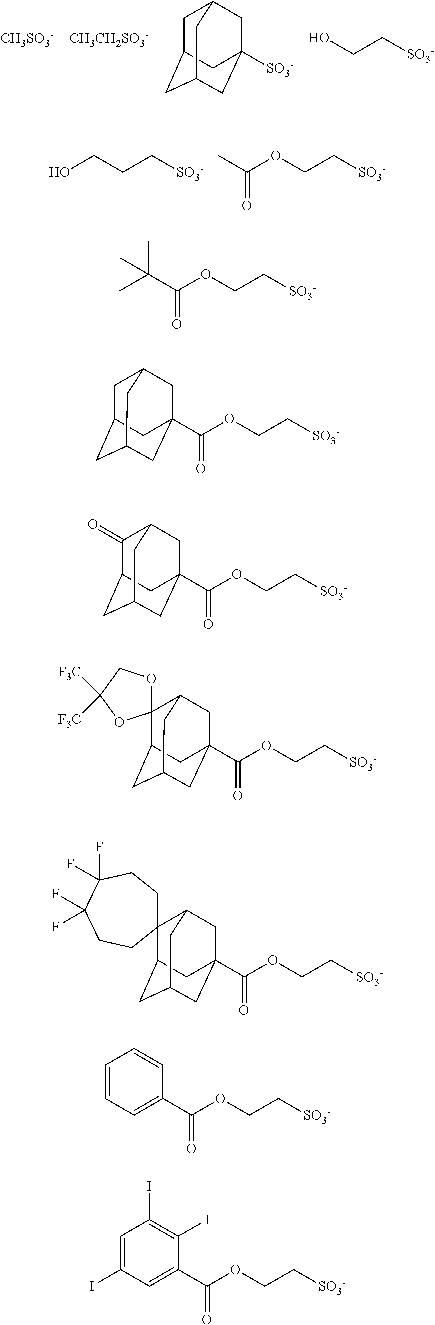

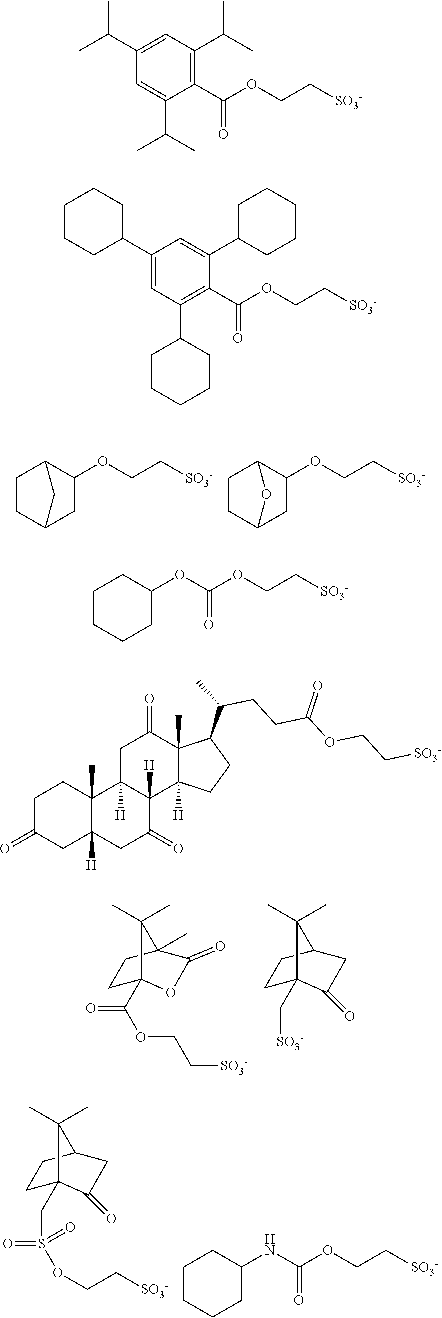

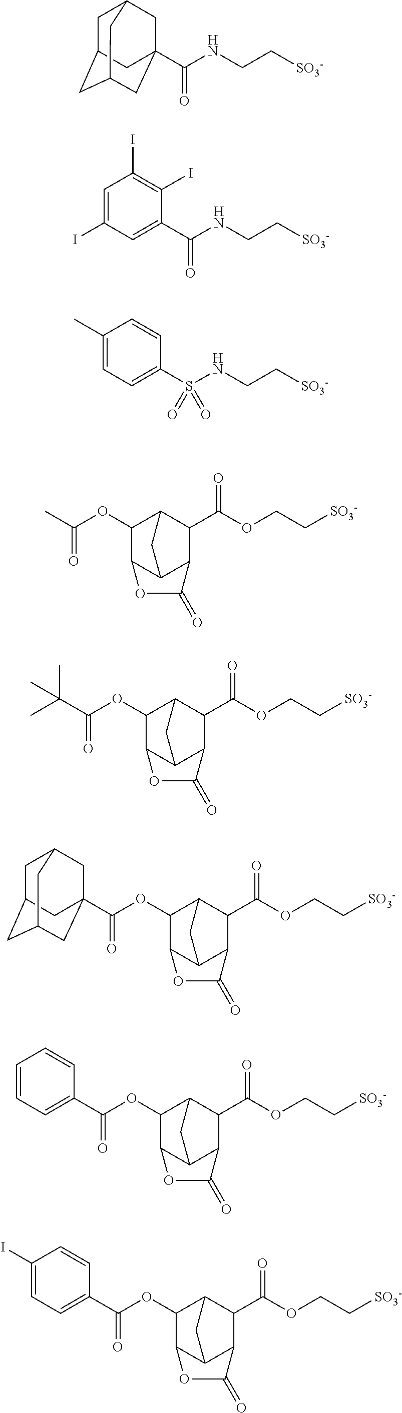

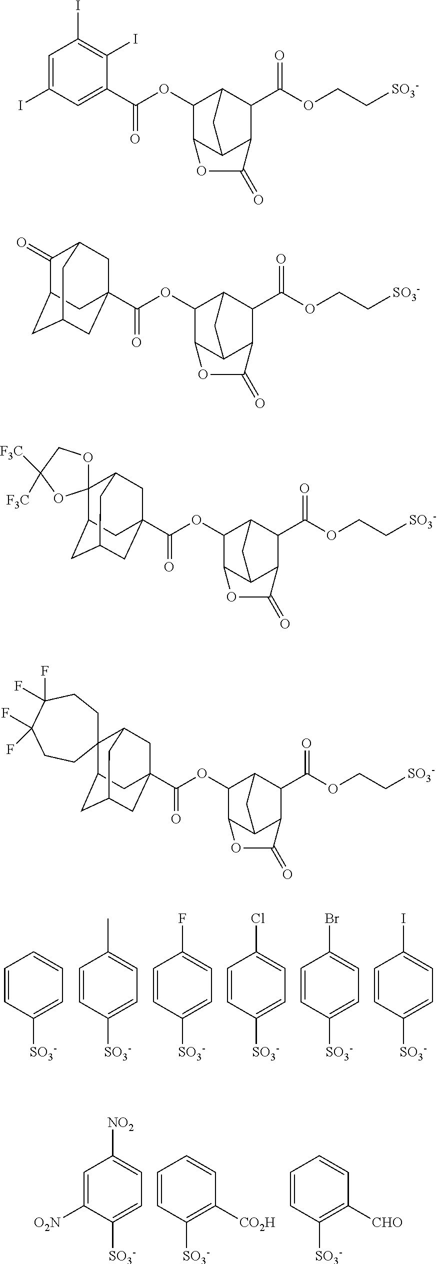

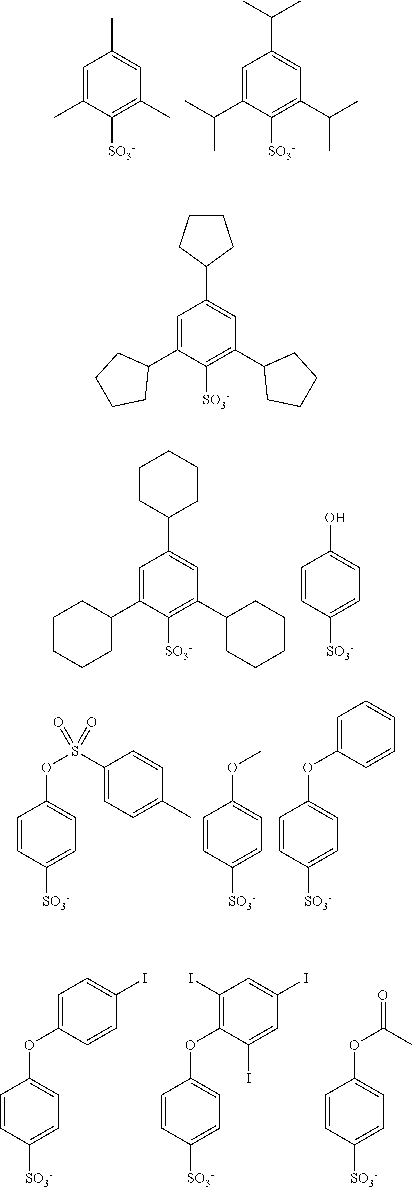

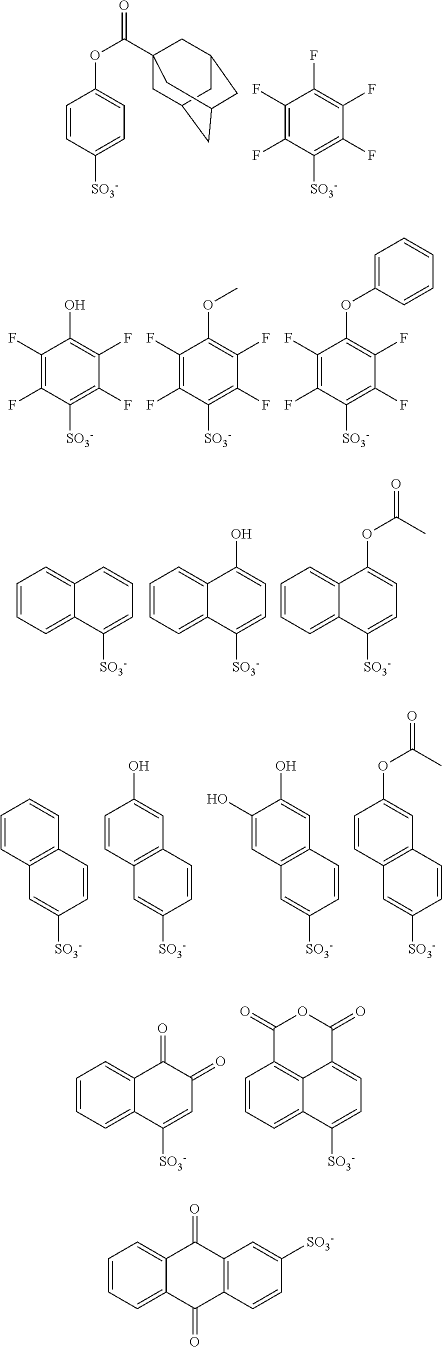

Examples of the anion in the onium salt having the formula (6) include those shown below, but not limited thereto.

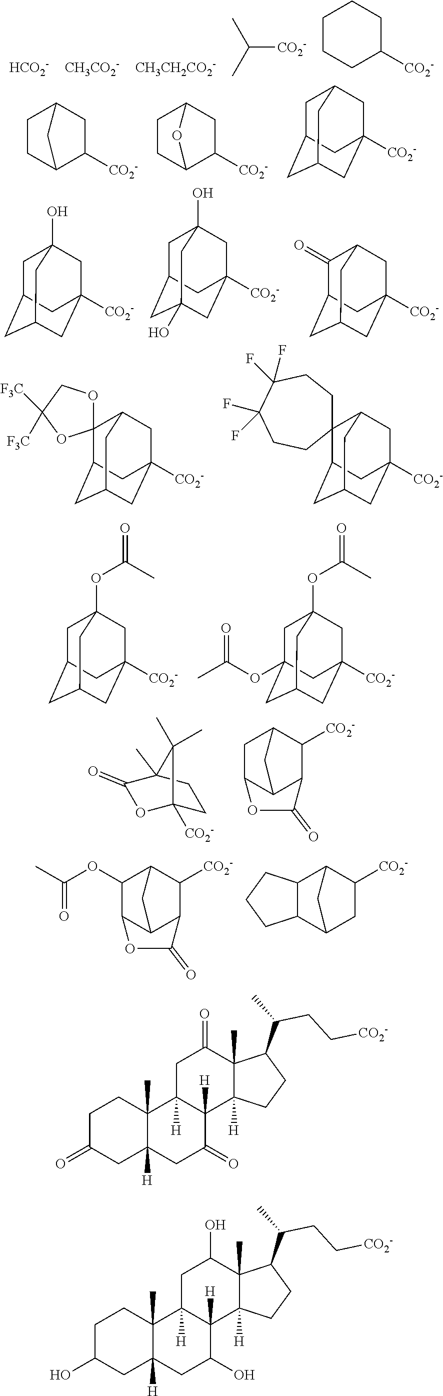

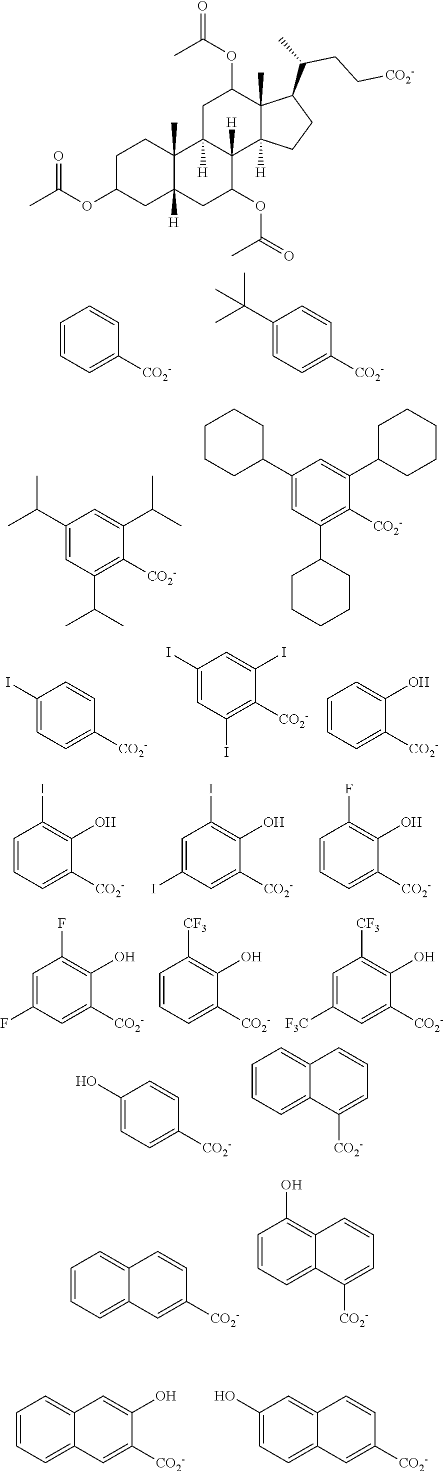

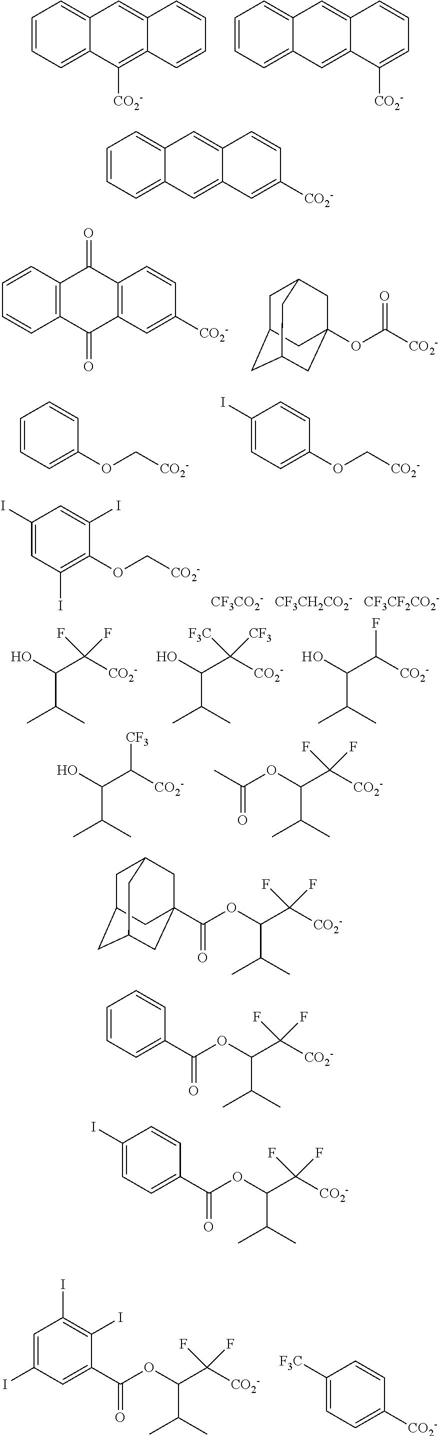

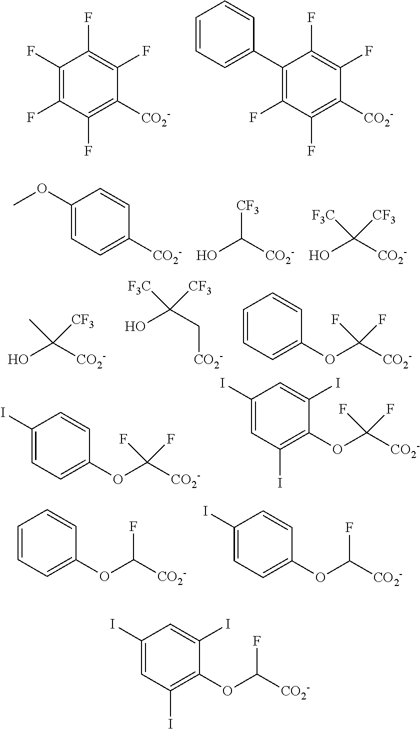

In the formulae (5) and (6), Mq⁺ is an onium cation. Examples of the onium cation include sulfonium, iodonium and ammonium cations. Examples of the sulfonium cation include those described as examples of the sulfonium cation in the formula (A), those described in JP-A 2024-003744, paragraphs [0102] to [0125], those described in WO 2024/128017, paragraphs [0044] to [0049], and those described in JP 7491173, paragraphs [0035] to [0046], and those described as examples of the sulfonium cation in the formula (d3), but not limited thereto. Examples of the iodonium cation include those described in JP-A 2024-000259, paragraph [0181], but not limited thereto. Examples of the ammonium cation include those described as examples of the ammonium cations having the formula (am-1).

Examples of the onium salt having the formula (5) or (6) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having the formula (5) or (6) functions as a quencher in the chemically amplified resist composition. because the counter anion of the onium salt is a conjugated base of a weak acid. As used herein, the weak acid indicates an acidity insufficient to deprotect an acid labile group from an acid labile group-containing unit in the base polymer. The onium salt having the formula (5) or (6) functions as a quencher when used in combination with an onium salt type photoacid generator having a conjugated base of a strong acid (typically sulfonic acid) as the counter anion. In a system using a mixture of an onium salt capable of generating a strong acid (typically sulfonic acid) and an onium salt capable of generating a weak acid (typically carboxylic acid), if the strong acid generated from the photoacid generator upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a lower catalysis, incurring apparent deactivation of the acid for enabling to control acid diffusion.

Also, as the other quencher (E), onium salts having sulfonium cation and phenoxide anion sites in a common molecule as described in JP 6848776, onium salts having sulfonium cation and carboxylate anion sites in a common molecule as described in JP 6583136 and JP-A 2020-200311, and onium salts having iodonium cation and carboxylate anion sites in a common molecule as described in JP 6274755 can also be used.

If a photoacid generator capable of generating a strong acid is an onium salt, an exchange from the strong acid generated upon exposure to high-energy radiation to a weak acid can take place as mentioned above, but it rarely happens that the weak acid generated upon exposure to high-energy radiation collides with the unreacted onium salt capable of generating a strong acid to induce a salt exchange. This is because of the phenomenon that an onium cation is more likely to form an ion pair with a stronger acid anion.

When the chemically amplified resist composition of the invention comprises the onium salt having the formula (5) or (6) as the other quencher (E), the amount thereof is preferably 0.1 to 20 parts by weight, more preferably 0.1 to 10 parts by weight per 80 parts by weight of the base polymer (B). As long as the amount of onium salt type quencher is in the range, a satisfactory resolution is available without a substantial lowering of sensitivity. The onium salt having the formula (5) or (6) can be used alone or in combination of two or more.

The chemically amplified resist composition of the invention may also comprise a nitrogen-containing compound as the other quencher (E). Examples of the nitrogen-containing compound include primary, secondary and tertiary amine compounds, specifically amine compounds having a hydroxy group, ether bond, ester bond, lactone ring, cyano group or sulfonate ester bond, as described in JP-A 2008-111103, paragraphs [0146]-[0164](U.S. Pat. No. 7,537,880), and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the chemically amplified resist composition of the invention comprises the nitrogen-containing compound as the other quencher (E), the amount thereof used is preferably 0.001 to 12 parts by weight, more preferably 0.01 to 8 parts by weight per 80 parts by weight of the base polymer (B). The nitrogen-containing compound may be used alone or in combination of two or more.

### (F) Surfactant

The chemically amplified resist composition of the invention may further comprise a surfactant as component (F). The surfactant (F) is preferably a surfactant which is insoluble or substantially insoluble in water and soluble in alkaline developer, or a surfactant which is insoluble or substantially insoluble in water and alkaline developer. For the surfactant, reference may be made to those described in JP-A 2010-215608 and JP-A 2011-016746.

Of the surfactants described in the patent documents cited herein, preferred surfactants which are insoluble or substantially insoluble in water and alkaline developer are surfactants FC-4430 (3M), Surflon® 5-381 (AGC Seimi Chemical Co., Ltd.), Olfine® E1004 (Nissin Chemical Co., Ltd.), and KH-20 and KH-30 (AGC Seimi Chemical Co., Ltd.). Oxetane ring-opened polymers having the formula (surf-1) are also preferred.

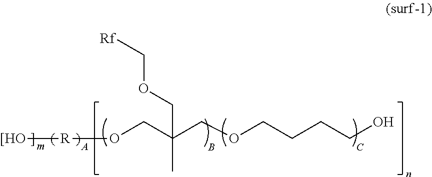

It is provided herein that R, Rf, A, B, C, m, and n are applied to only the formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

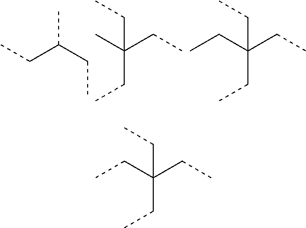

Herein the broken line is a valence bond. These formulae are partial structures derived from glycerol, trimethylol ethane, trimethylol propane, and pentaerythritol, respectively.

Among them, 1,4-butylene and 2,2-dimethyl-1,3-propylene are preferred.

Rf is trifluoromethyl or pentafluoroethyl, preferably trifluoromethyl. m is an integer of 0 to 3, n is an integer of 1 to 4, and the sum of m and n, which represents the valence of R, is an integer of 2 to 4. A is 1. B is an integer of 2 to 25, preferably an integer of 4 to 20. C is an integer of 0 to 10, preferably an integer of 0 or 1. Note that the formula (surf-1) does not prescribe the arrangement of respective constituent units while they may be arranged either blockwise or randomly. For the production of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during aqueous alkaline development following exposure or post exposure bake (PEB), and thus forms few or no foreign matter which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Examples of the polymeric surfactant include those containing repeat units of at least one type selected from the formulae (6A) to (6E).

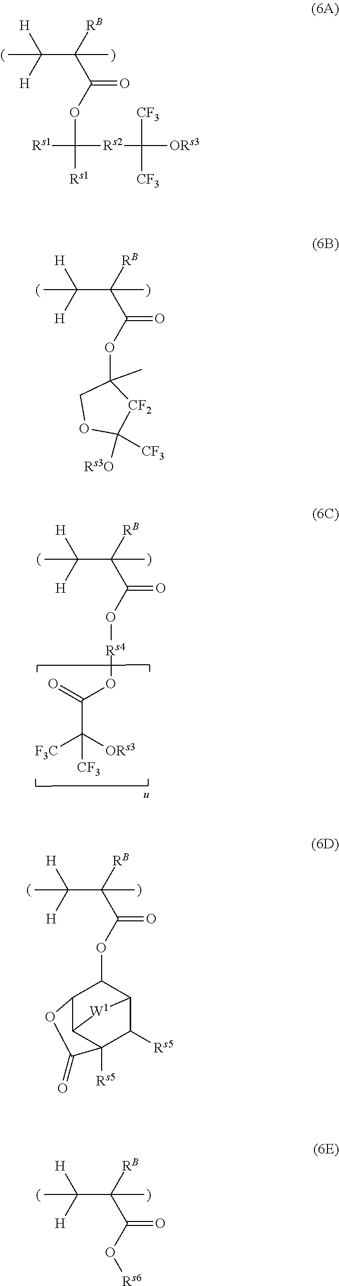

In the formulae (6A) to (6E), R^{B} is hydrogen, fluorine, methyl or trifluoromethyl. W¹ is —CH₂—, —CH₂CH₂—, —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group, and u is 1, 2, or 3. R^{s5} is each independently hydrogen or a group represented by —C(═O)—O—R^{sa} wherein R^{sa} is a C₁-C₂₀ fluorinated hydrocarbyl group. R^{s6} is a C1-Cis hydrocarbyl or fluorinated hydrocarbyl group in which an ether bond or carbonyl moiety may intervene in a carbon-carbon bond.

The C₁-C₁₀ hydrocarbyl group represented by R^{s1} is preferably saturated and may be straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl, and n-decyl, and C₃-C₁₀ cyclic saturated hydrocarbyl groups such as cyclopropyl, cyclobutyl, cyclopentyl, cyclohexyl, adamantyl, and norbornyl. Of these, C₁-C₆ hydrocarbyl groups are preferred.

The hydrocarbylene group represented by R^{s2} is preferably saturated and may be straight, branched or cyclic. Examples thereof include methylene, ethylene, propylene, butylene, and pentylene.

The hydrocarbyl group represented by R^{s3} or R^{s6} may be saturated or unsaturated and straight, branched or cyclic. Examples thereof include saturated hydrocarbyl groups, and aliphatic unsaturated hydrocarbyl groups such as alkenyl and alkynyl groups, with the saturated hydrocarbyl groups being preferred. Examples of the saturated hydrocarbyl groups include those exemplified for the hydrocarbyl group represented by R^{s1} as well as undecyl, dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms.

In these groups, an ether bond or carbonyl moiety may intervene in a carbon-carbon bond as mentioned above.

Examples of the acid labile group represented by R^{s3} include groups of the above formulae (AL-3) to (AL-5), trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{s4} may be straight, branched or cyclic, and examples thereof include the foregoing hydrocarbyl or fluorinated hydrocarbyl groups from which “u” number of hydrogen atoms are eliminated.

The fluorinated hydrocarbyl group represented by R^{sa} is preferably saturated and may be straight, branched or cyclic. Examples thereof include the foregoing hydrocarbyl groups in which some or all of the hydrogen atoms are substituted by fluorine atoms.

Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having any of the formulae (6A) to (6E) include those shown below, but not limited thereto. In the formulae, RB is as defined above.

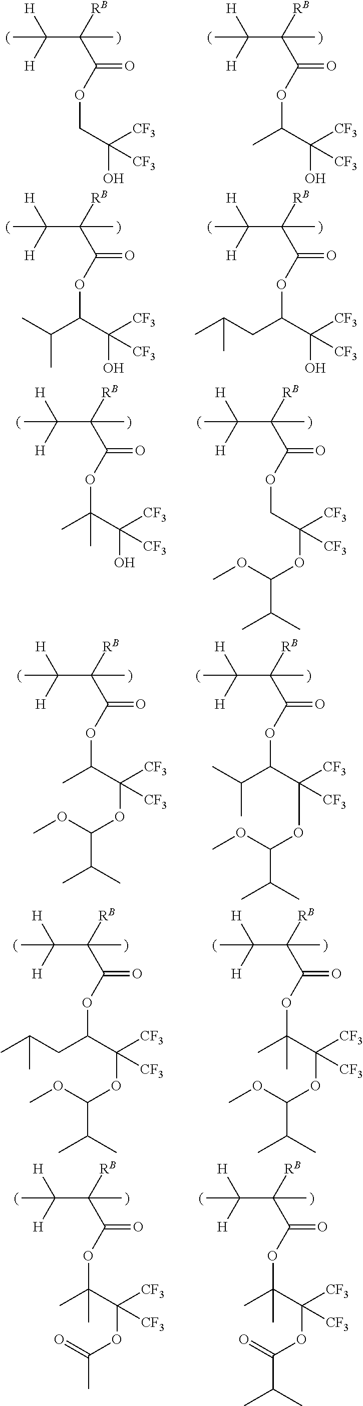

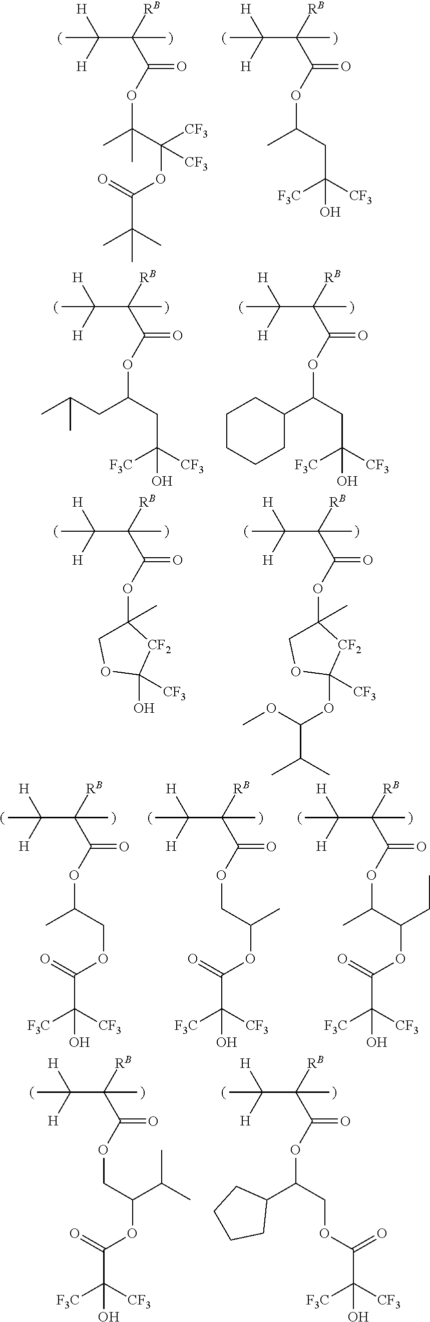

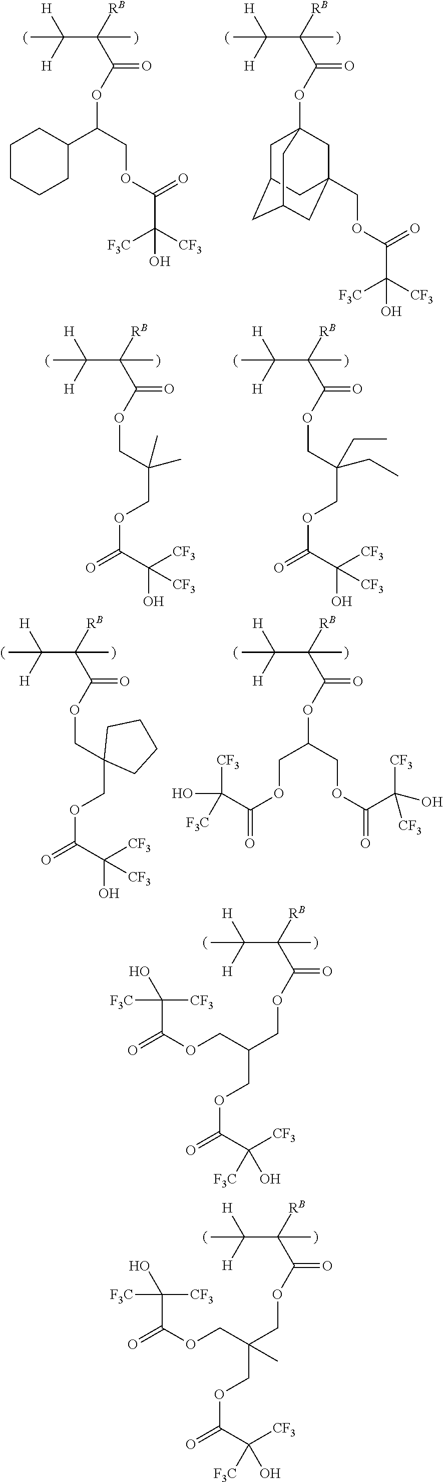

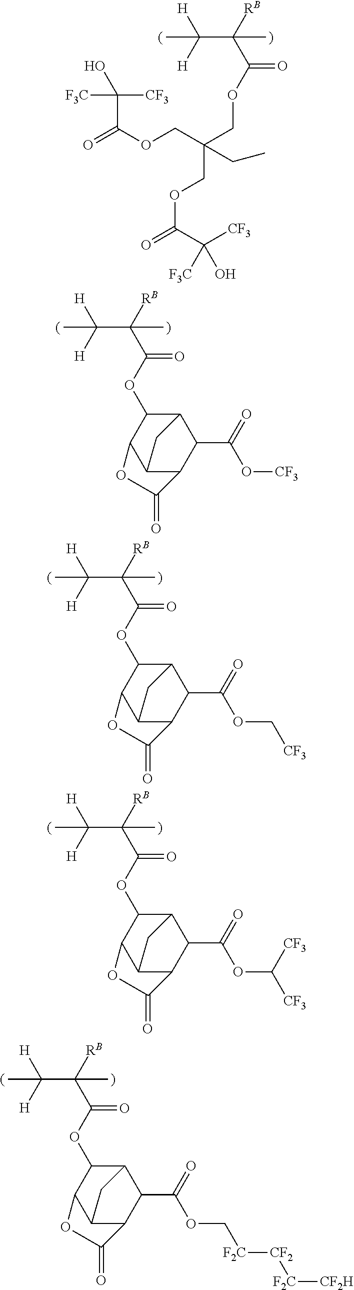

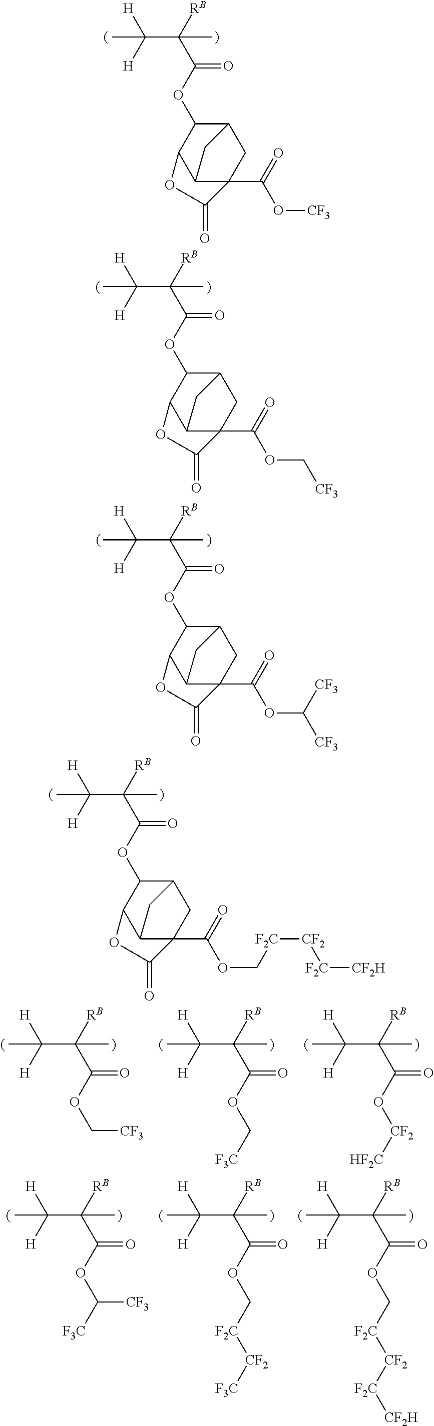

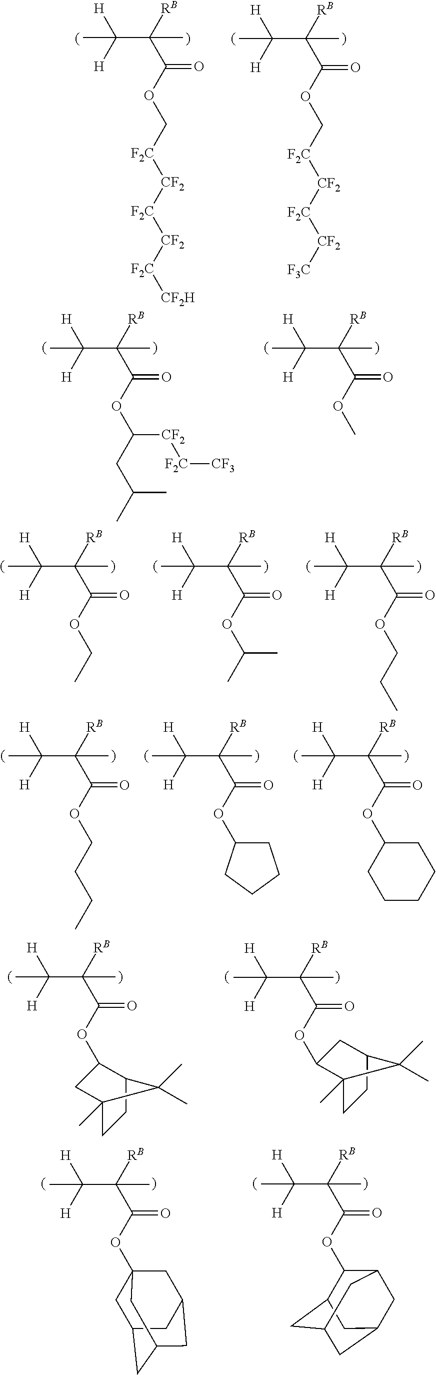

The polymeric surfactant may further contain repeat units other than the repeat units having the formulae (6A) to (6E). Examples of the other repeat units include those derived from methacrylic acid and α-trifluoromethylacrylic acid derivatives. In the polymeric surfactant, the content of the repeat units having the formulae (6A) to (6E) is preferably at least 20 mol %, more preferably at least 60 mol %, most preferably 100 mol % of the overall repeat units.

The polymeric surfactant has a Mw of preferably 1,000 to 500,000, more preferably 3,000 to 100,000, and a Mw/Mn of preferably 1.0 to 2.0, more preferably 1.0 to 1.6.

The polymeric surfactant may be synthesized, for example, by dissolving an unsaturated bond-containing monomer or monomers providing repeat units having the formulae (6A) to (6E) and optionally other repeat units in an organic solvent, adding a radical initiator, and heating for polymerization. Examples of the organic solvents used in the polymerization include toluene, benzene, THF, diethyl ether, and dioxane. Examples of the polymerization initiator include AIBN, 2,2′-azobis(2,4-dimethylvaleronitrile), dimethyl 2,2-azobis(2-methylpropionate), benzoyl peroxide, and lauroyl peroxide. Preferably, the reaction temperature is 50 to 100° C. and the reaction time is 4 to 24 hours. The acid labile group that has been incorporated in the monomer may be kept as such, or polymerization may be followed by protection or partial protection.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

When the chemically amplified resist composition of the invention comprises the surfactant (F), the amount thereof is preferably 0.1 to 50 parts by weight, more preferably 0.5 to 10 parts by weight per 80 parts by weight of the base polymer (B). At least 0.1 parts by weight of the surfactant (F) is effective in improving the receding contact angle with water of the resist film at its surface. Up to 50 parts by weight of the surfactant (F) is effective in forming a resist film having a low rate of dissolution in a developer and capable of maintaining the height of a small-size pattern formed therein. The surfactant (F) may be used singly or in combination of two or more kinds thereof.

### (G) Other Components

The chemically amplified resist composition of the invention may further comprise (G) other components, for example, a compound which is decomposed with an acid to generate another acid (i.e., acid amplifier compound), an organic acid derivative, a fluorinated alcohol, and a compound having a Mw of up to 3,000 which changes its solubility in developer under the action of an acid (i.e., dissolution inhibitor). For the acid amplifier compound, reference may be made to compounds described in JP-A 2009-269953 and JP-A 2010-215608. When the chemically amplified resist composition of the invention comprises the acid amplifier compound, the amount thereof is preferably 0 to 5 parts by weight, more preferably 0 to 3 parts by weight per 80 parts by weight of the base polymer (B). An extra amount of the acid amplifier compound can make the acid diffusion control difficult and cause degradations to resolution and pattern profile. For the organic acid derivative, fluorinated alcohol, and dissolution inhibitor, reference should be made to compounds described in JP-A 2009-269953 and JP-A 2010-215608.

### Pattern Forming Process

A pattern forming process of the invention comprises the steps of applying the chemically amplified resist composition defined above onto a substrate to form a resist film thereon, exposing the resist film to high-energy radiation, and developing the exposed resist film in a developer.

The substrate used herein may be a substrate for integrated circuitry fabrication, e.g., Si, SiO₂, SiN, SiON, TiN, WSi, BPSG, SOG, organic antireflective film, etc. or a substrate for mask circuitry fabrication, e.g., Cr, CrO, CrON, MoSi₂, SiO₂, etc.

The chemically amplified resist composition is applied onto a substrate by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of 0.05 to 2 μm.

Examples of the high-energy radiation used for exposure of the resist film include KrF excimer laser, ArF excimer laser, EB, and EUV. On use of KrF excimer laser, ArF excimer laser or EUV, the resist film is exposed through a mask having a desired pattern, in a dose of preferably 1 to 200 mJ/cm², more preferably 10 to 100 mJ/cm². On use of EB, a pattern may be written directly or through a mask having the desired pattern, in a dose of preferably 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed. In this case, a protective film which is insoluble in water may be also used.

While the water-insoluble protective film serves to prevent any components from being leached out of the resist film and to improve water sliding on the film surface, it is generally divided into two types. The first type is an organic solvent-strippable protective film which must be stripped, prior to aqueous alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, more preferably at 80 to 140° C. for 1 to 3 minutes.

The resist film is then developed with a developer in the form of an aqueous alkaline solution, for example, preferably 0.1 to 5 wt %, more preferably 2 to 3 wt % aqueous solution of tetramethylammonium hydroxide (TMAH) for preferably 0.1 to 3 minutes, more preferably 0.5 to 2 minutes by conventional techniques such as dip, puddle and spray techniques. In this way, the exposed region of the resist film is dissolved away, and a desired pattern is formed on the substrate.

Any desired step may be added to the pattern forming process. For example, after the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvent may be used alone or in admixture of two or more.

### EXAMPLES

Synthesis Examples, Examples and Comparative Examples are given below by way of illustration and not by way of limitation. Analysis is made using the following instrument.

### MALDI TOF-MS: S3000 by JEOL Ltd.

### [1]Synthesis of Onium Salts

### Example 1-1

### Synthesis of Onium Salt PDQ-1

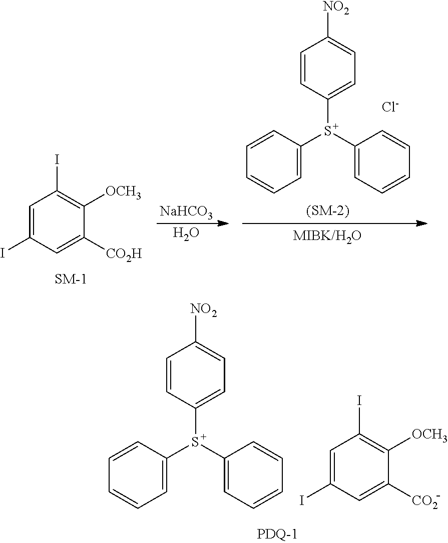

### (1) Synthesis of Onium Salt PDQ-1

In a reactor under nitrogen atmosphere, 4.0 g of Reactant SM-1, 1.0 g of sodium hydrogencarbonate and 20 g of water were mixed and stirred at an internal temperature of 45° C. for 1 hour. The reaction solution was cooled to room temperature. Thereafter, 15.0 g of Reactant SM-2 (corresponding to 0.012 mol) and 30 g of methyl isobutyl ketone were added to the solution, which was stirred at room temperature for 30 minutes. At the end of stirring, the organic layer was taken out, washed with water, and concentrated under reduced pressure. The concentrate was purified by silica gel chromatography, obtaining the target Onium Salt PDQ-1 as oily matter (amount 5.8 g, yield 82%).

PDQ-1 was analyzed by TOF-MS, with the data shown below.

### MALDI TOF-MS:

positive M⁺ 308 (corresponding to C₁₈H₁₄NO₂S⁺)
negative M⁻ 403 (corresponding to C₈H₅I₂O₃⁻—)

### Examples 1-2 to 1-7

### Synthesis of Onium Salts PDQ-2 to PDQ-7

Onium salts PDQ-2 to PDQ-7 having the formulae were synthesized using corresponding reactants and well-known organic synthesis reactions.

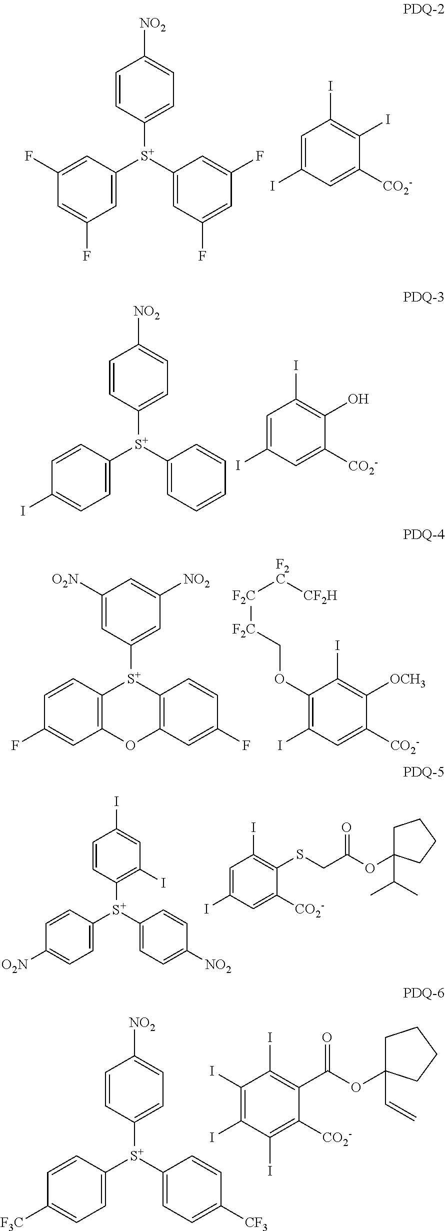

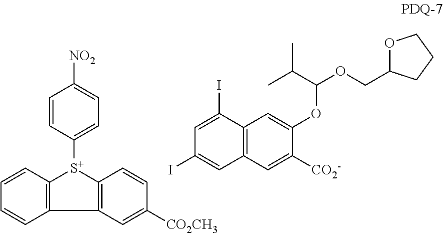

### Synthesis Example

### Synthesis of Base Polymers P-1 to P-5

Base polymers P-1 to P-5 were synthesized by combining monomers, performing copolymerization reaction in MEK solvent, pouring the reaction solution to hexane for precipitation, washing the solid precipitate with hexane, isolation and drying. The base polymers were analyzed for composition by ¹H-NMR spectroscopy and for Mw and Mw/Mn by GPC versus polystyrene standards using DMF solvent.

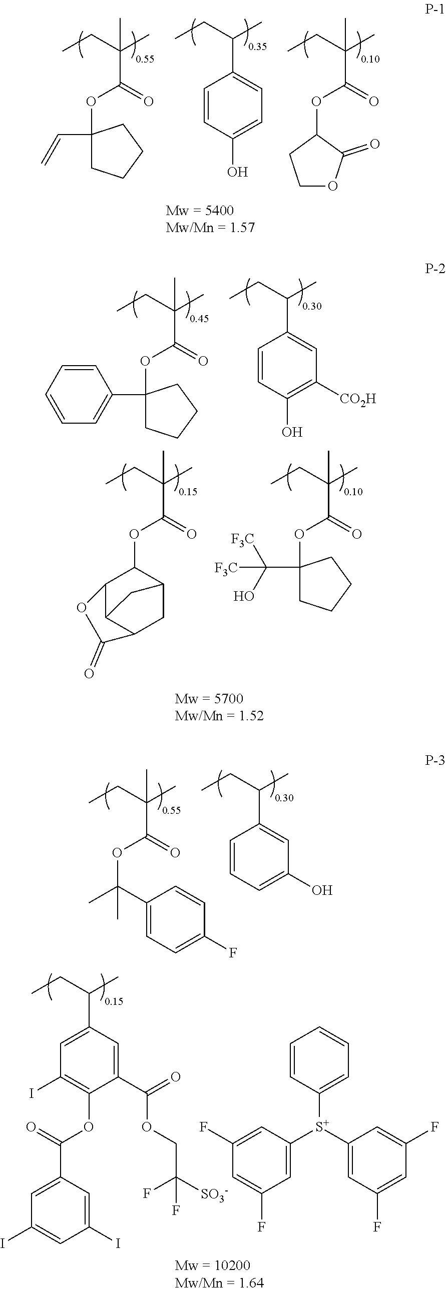

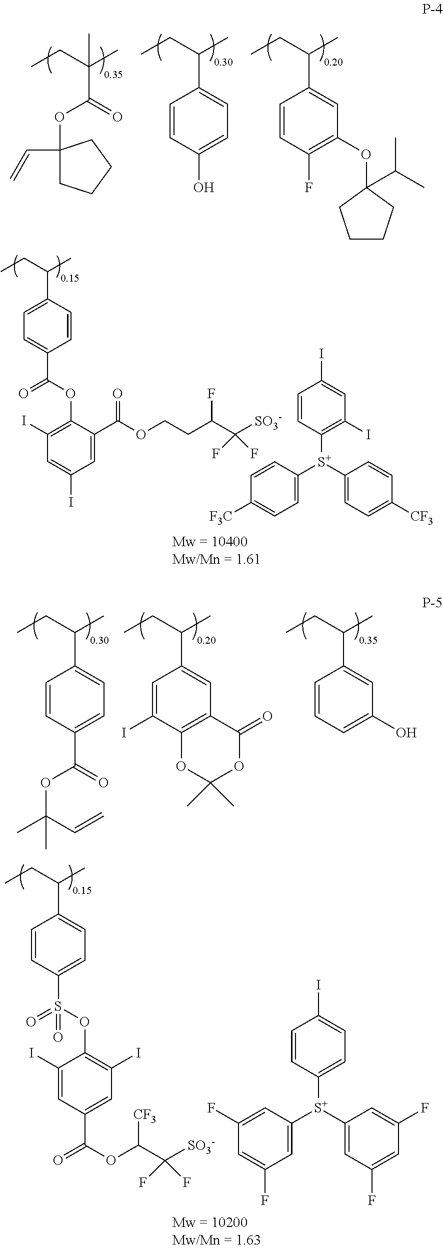

### [3]Preparation of Chemically Amplified Resist Composition

### Examples 2-1 to 2-30 and Comparative Examples 1-1 to 1-20

Chemically amplified resist compositions (R-1 to R-30, CR-1 to CR-20) in solution form were prepared by dissolving a quencher (PDQ-1 to PDQ-7) or comparative quencher (PDQ-A to PDQ-D), photoacid generator (PAG-X and PAG-Y), base polymer (P-1 to P-5), and other quencher (AQ-1 and AQ-2) in a solvent containing 0.01 wt % of surfactant A (Omnova Solutions, Inc.) in accordance with the formulation shown in Tables 1 and 2, and filtering through a Teflon® filter with a pore size of 0.2 μm.

TABLE 1

| | | | Base | | Photoacid | Other | | | |
|---|---|---|---|---|---|---|---|---|---|
| | | Resist | polymer | Quencher | generator | quencher | Solvent 1 | Solvent 2 | Solvent 3 |
| | | composition | (pbw) | (pbw) | (pbw) | (pbw) | (pbw) | (pbw) | (pbw) |
| Example | 2-1 | R-1 | P-1 (80) | PDQ-1 (5) | PAG-X (15) | — | PGMEA (2250) | EL (2800) | DAA (550) |
| | 2-2 | R-2 | P-1 (80) | PDQ-2 (5) | PAG-X (15) | — | PGMEA (2250) | EL (2800) | DAA (550) |
| | 2-3 | R-3 | P-1 (80) | PDQ-3 (5) | PAG-X (15) | — | PGMEA (2250) | EL (2800) | DAA (550) |
| | 2-4 | R-4 | P-1 (80) | PDQ-4 (5) | PAG-X (15) | — | PGMEA (2250) | EL (2800) | DAA (550) |
| | 2-5 | R-5 | P-1 (80) | PDQ-5 (5) | PAG-X (15) | — | PGMEA (2250) | EL (2800) | DAA (550) |
| | 2-6 | R-6 | P-1 (80) | PDQ-6 (5) | PAG-X (15) | — | PGMEA (2250) | EL (2800) | DAA (550) |
| | 2-7 | R-7 | P-1 (80) | PDQ-7 (5) | PAG-X (15) | — | PGMEA (2250) | EL (2800) | DAA (550) |
| | 2-8 | R-8 | P-1 (80) | PDQ-1 (5) | PAG-Y (15) | — | PGMEA (2250) | EL (2800) | DAA (550) |
| | 2-9 | R-9 | P-1 (80) | PDQ-1 (2.5) | PAG-X (15) | AQ-1 (2.5) | PGMEA (2250) | EL (2800) | DAA (550) |
| | 2-10 | R-10 | P-1 (80) | PDQ-2 (3) | PAG-Y (15) | AQ-2 (2) | PGMEA (2250) | EL (2800) | DAA (550) |
| | 2-11 | R-11 | P-2 (80) | PDQ-1 (5) | PAG-X (14) | — | PGMEA (2250) | EL (2800) | DAA (550) |
| | 2-12 | R-12 | P-2 (80) | PDQ-2 (5) | PAG-X (15) | — | PGMEA (2250) | EL (2800) | DAA (550) |
| | 2-13 | R-13 | P-2 (80) | PDQ-4 (5) | PAG-Y (14) | — | PGMEA (2250) | EL (2800) | DAA (550) |
| | 2-14 | R-14 | P-2 (80) | PDQ-6 (5) | PAG-Y (15) | — | PGMEA (2250) | EL (2800) | DAA (550) |
| | 2-15 | R-15 | P-2 (80) | PDQ-1 (4) | PAG-X (15) | AQ-1 (2) | PGMEA (2250) | EL (2800) | DAA (550) |
| | 2-16 | R-16 | P-3 (80) | PDQ-1 (5) | — | — | PGMEA (2250) | EL (2800) | DAA (550) |
| | 2-17 | R-17 | P-3 (80) | PDQ-2 (5) | — | — | PGMEA (2250) | EL (2800) | DAA (550) |
| | 2-18 | R-18 | P-3 (80) | PDQ-3 (5) | — | — | PGMEA (2250) | EL (2800) | DAA (550) |
| | 2-19 | R-19 | P-3 (80) | PDQ-4 (5) | PAG-X (5) | — | PGMEA (2250) | EL (2800) | DAA (550) |
| | 2-20 | R-20 | P-3 (80) | PDQ-2 (3) | — | AQ-1 (2) | PGMEA (2250) | EL (2800) | DAA (550) |
| | 2-21 | R-21 | P-4 (80) | PDQ-1 (5) | — | — | PGMEA (2250) | EL (2800) | DAA (550) |
| | 2-22 | R-22 | P-4 (80) | PDQ-2 (5) | — | — | PGMEA (2250) | EL (2800) | DAA (550) |
| | 2-23 | R-23 | P-4 (80) | PDQ-5 (5) | — | — | PGMEA (2250) | EL (2800) | DAA (550) |
| | 2-24 | R-24 | P-4 (80) | PDQ-6 (6) | PAG-Y (5) | — | PGMEA (2250) | EL (2800) | DAA (550) |
| | 2-25 | R-25 | P-4 (80) | PDQ-2 (5) | — | — | PGMEA (2250) | EL (2800) | DAA (550) |
| | 2-26 | R-26 | P-5 (80) | PDQ-1 (5) | — | — | PGMEA (2250) | EL (2800) | DAA (550) |
| | 2-27 | R-27 | P-5 (80) | PDQ-2 (5) | — | — | PGMEA (2250) | EL (2800) | DAA (550) |
| | 2-28 | R-28 | P-5 (80) | PDQ-7 (3) | PAG-Y (5) | AQ-2 (2) | PGMEA (2250) | EL (2800) | DAA (550) |
| | 2-29 | R-29 | P-5 (80) | PDQ-3 (5) | — | — | PGMEA (2250) | EL (2800) | DAA (550) |
| | 2-30 | R-30 | P-5 (80) | PDQ-2 (2.5) | — | AQ-1 (2.5) | PGMEA (2250) | EL (2800) | DAA (550) |

TABLE 2

| | | | Base | | Photoacid | Other | | | |
|---|---|---|---|---|---|---|---|---|---|
| | | Resist | polymer | Quencher | generator | quencher | Solvent 1 | Solvent 2 | Solvent 3 |
| | | composition | (pbw) | (pbw) | (pbw) | (pbw) | (pbw) | (pbw) | (pbw) |
| Comparative | 1-1 | CR-1 | P-1 (80) | PDQ-A (5) | PAG-X (15) | — | PGMEA (2250) | EL (2800) | DAA (550) |
| Example | 1-2 | CR-2 | P-1 (80) | PDQ-B (5) | PAG-X (15) | — | PGMEA (2250) | EL (2800) | DAA (550) |
| | 1-3 | CR-3 | P-1 (80) | PDQ-C (5) | PAG-X (15) | — | PGMEA (2250) | EL (2800) | DAA (550) |
| | 1-4 | CR-4 | P-1 (80) | PDQ-D (5) | PAG-X (15) | — | PGMEA (2250) | EL (2800) | DAA (550) |
| | 1-5 | CR-5 | P-1 (80) | PDQ-A (5) | PAG-Y (15) | — | PGMEA (2250) | EL (2800) | DAA (550) |
| | 1-6 | CR-6 | P-1 (80) | PDQ-B (5) | PAG-Y (15) | — | PGMEA (2250) | EL (2800) | DAA (550) |
| | 1-7 | CR-7 | P-1 (80) | PDQ-C (2.5) | PAG-X (15) | AQ-1 (2.5) | PGMEA (2250) | EL (2800) | DAA (550) |
| | 1-8 | CR-8 | P-1 (80) | PDQ-D (3) | PAG-Y (15) | AQ-2 (2) | PGMEA (2250) | EL (2800) | DAA (550) |
| | 1-9 | CR-9 | P-2 (80) | PDQ-A (5) | PAG-X (15) | — | PGMEA (2250) | EL (2800) | DAA (550) |
| | 1-10 | CR-10 | P-2 (80) | PDQ-B (5) | PAG-X (15) | — | PGMEA (2250) | EL (2800) | DAA (550) |
| | 1-11 | CR-11 | P-2 (80) | PDQ-C (4) | PAG-X (15) | AQ-1 (2) | PGMEA (2250) | EL (2800) | DAA (550) |
| | 1-12 | CR-12 | P-3 (80) | PDQ-A (5) | — | — | PGMEA (2250) | EL (2800) | DAA (550) |
| | 1-13 | CR-13 | P-3 (80) | PDQ-C (5) | — | — | PGMEA (2250) | EL (2800) | DAA (550) |
| | 1-14 | CR-14 | P-3 (80) | PDQ-D (3) | — | AQ-1 (2) | PGMEA (2250) | EL (2800) | DAA (550) |
| | 1-15 | CR-15 | P-4 (80) | PDQ-A (5) | — | — | PGMEA (2250) | EL (2800) | DAA (550) |
| | 1-16 | CR-16 | P-4 (80) | PDQ-D (5) | — | — | PGMEA (2250) | EL (2800) | DAA (550) |
| | 1-17 | CR-17 | P-4 (80) | PDQ-C (6) | PAG-Y (5) | — | PGMEA (2250) | EL (2800) | DAA (550) |
| | 1-18 | CR-18 | P-5 (80) | PDQ-A (5) | — | — | PGMEA (2250) | EL (2800) | DAA (550) |
| | 1-19 | CR-19 | P-5 (80) | PDQ-B (3) | PAG-Y (5) | AQ-2 (2) | PGMEA (2250) | EL (2800) | DAA (550) |
| | 1-20 | CR-20 | P-5 (80) | PDQ-C (5) | — | — | PGMEA (2250) | EL (2800) | DAA (550) |

The solvents, photoacid generators PAG-X and PAG-Y, comparative quenchers PDQ-A to PDQ-D, other quenchers AQ-1 and AQ-2, and surfactant A in Tables 1 and 2 are identified below.
Solvents:
   PGMEA (propylene glycol monomethyl ether acetate)
   EL (ethyl lactate)
   DAA (diacetone alcohol)
Photoacid generators: PAG-X and PAG-Y

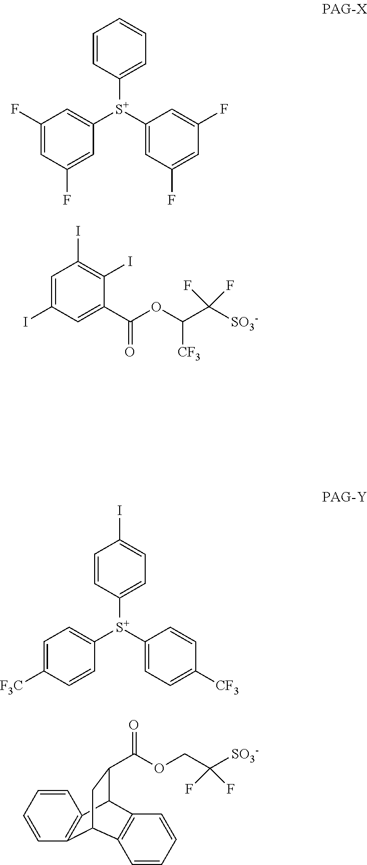

Comparative quenchers: PDQ-A to PDQ-D

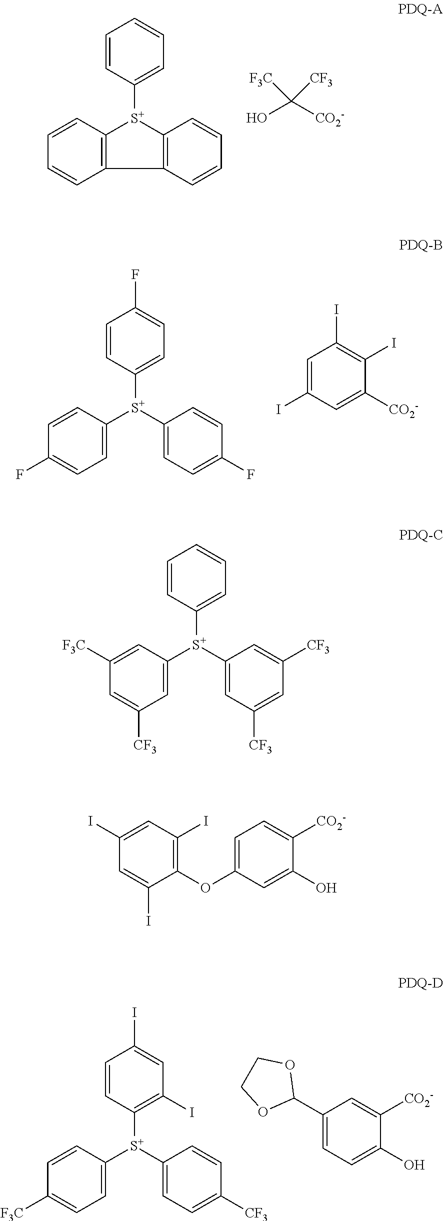

Other quenchers: AQ-1 and AQ-2

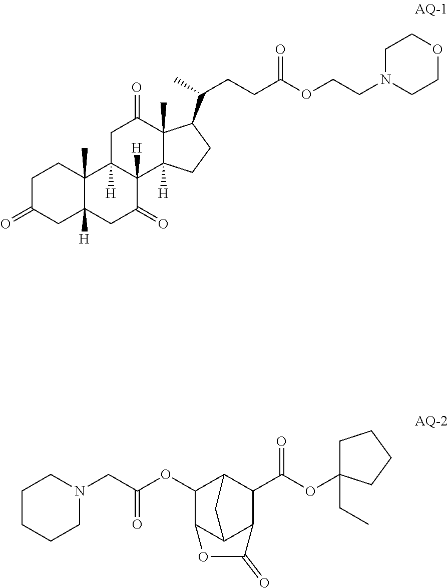

Surfactants A:
   3-methyl-3-(2,2,2-trifluoroethoxymethyl)-oxetane/tetrahydrofuran/
   2,2-dimethyl-1,3-propane diol copolymer (manufactured by Omnova Solutions, Inc.)

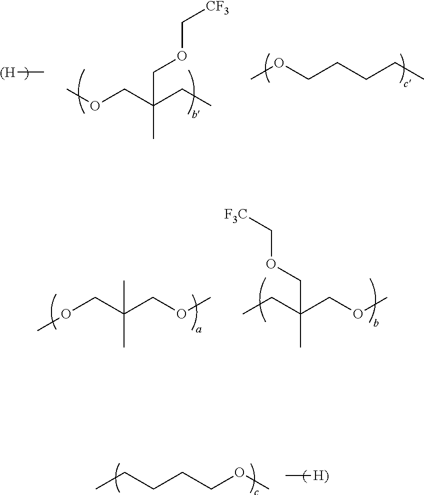

$$a : ( b + b ’ ) : ( c + c ’ ) = 1 : 4 - 7 : 0.01 - 1 ( molar ratio )$$

$$Mw = 1 , 500$$

### [4]EUV Lithography Test (1)

### Examples 3-1 to 3-30 and Comparative Examples 2-1 to 2-20

Each of the chemically amplified resist compositions (R-1 to R-30, CR-1 to CR-20) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3400 (ASML, NA 0.33, σ 0.9/0.6, dipole illumination), the resist film was exposed to EUV through a mask bearing an LS pattern having a size of 18 nm and a pitch of 36 nm (on-wafer size) while varying the dose and focus (dose pitch: 1 mJ/cm², focus pitch: 0.020 μm). The resist film was baked (PEB) on a hotplate at the temperature shown in Tables 4 and 5 for 60 seconds and puddle developed in a 2.38 wt % TMAH aqueous solution for 30 seconds, rinsed with a rinse fluid containing surfactant, and spin dried to form a positive pattern. puddle developed in a 2.38 wt % TMAH aqueous solution for 30 seconds, rinsed with a rinse fluid containing surfactant, and spin dried to form a positive pattern.

The LS pattern as developed was observed under CD-SEM (CG6300, Hitachi High-Technologies Corp.) whereupon sensitivity, EL, LWR, depth of focus (DOF) and collapse limit were evaluated by the following methods. In addition, development defects of the obtained LS pattern were evaluated. The results are shown in Tables 3 and 4.

### [Evaluation of Sensitivity]

The optimum dose Eₒₚ(mJ/cm²) which provided an LS pattern with a line width of 18 nm and a pitch of 36 nm was determined as an index of sensitivity. A smaller value indicates a higher sensitivity.

### [Evaluation of EL]

The exposure dose which provided an LS pattern with a space width of 18 nm±10% (16.2 to 19.8 nm) was determined. EL (%) is calculated from the exposure doses according to the following equation:

$$EL ( \% ) = ( \vert {E}_{1} - {E}_{2} \vert / Eop ) \times 100$$

wherein E₁ is an optimum exposure dose which provides an LS pattern with a line width of 16.2 nm and a pitch of 36 nm, E₂ is an optimum exposure dose which provides an LS pattern with a line width of 19.8 nm and a pitch of 36 nm, and Eop is an optimum exposure dose which provides an LS pattern with a line width of 18 nm and a pitch of 36 nm. A greater value indicates better performance.

### [Evaluation of LWR]

For the LS pattern formed by exposure at the optimum dose Eₒₚ, the line width was measured at 10 longitudinally spaced apart points, from which a 3-fold value (3σ) of the standard deviation (σ) was determined and reported as LWR. A smaller value of 3σ indicates a pattern having small roughness and uniform line width.

### [Evaluation of DOF]

As an index of depth of focus, a range of focus which provided an LS pattern with a size of 18 nm±10% (i.e., 16.2 to 19.8 nm) was determined. A greater value indicates a wider depth of focus.

### [Evaluation of Line Pattern Collapse Limit]

For the LS pattern formed by exposure at the dose corresponding to the optimum focus, the line width was measured at 10 longitudinally spaced apart points. The minimum line width above which lines could be resolved without collapse was determined and reported as collapse limit. A smaller value indicates better collapse limit.

### [Evaluation of Development Defect]

Using a defect inspection apparatus KLA2360 (trade name) manufactured by KLA-Tencor Corporation, the LS pattern with a line width of 18 nm and a pitch of 36 nm formed with the optimum dose, the pixel size in the defect inspection apparatus was set at 0.16 μm and the threshold value at 20. Defects extracted from differences generated by superimposition between a comparative image and the pixel unit were detected, and the number of defects per unit area (defects/cm²) was calculated. Thereafter, development defects were classified and extracted from all defects by defect review, and the number of development defects per unit area (defects/cm²) was calculated. The evaluation marks A, B, C and D were given when the value was less than 0.5, 0.5 or more and less than 1.0, 1.0 or more and less than 5.0, and 5.0 or more, respectively. The smaller the value, the more favorable the performance exhibited.

TABLE 3

| | | | | Optimum | | | | | |
|---|---|---|---|---|---|---|---|---|---|
| | | | PEB | exposure | | | | Collapse | |
| | | Resist | temp. | dose | EL | LWR | DOF | limit | Development |
| | | composition | (° C.) | (mJ/cm²) | (%) | (nm) | (nm) | (nm) | defects |
| Example | 3-1 | R-1 | 100 | 32 | 18 | 2.4 | 110 | 11.1 | A |
| | 3-2 | R-2 | 100 | 33 | 18 | 2.5 | 120 | 11.3 | A |
| | 3-3 | R-3 | 100 | 33 | 17 | 2.3 | 110 | 10.9 | A |
| | 3-4 | R-4 | 95 | 34 | 17 | 2.3 | 110 | 11.2 | A |
| | 3-5 | R-5 | 100 | 33 | 18 | 2.4 | 120 | 11.2 | A |
| | 3-6 | R-6 | 100 | 33 | 18 | 2.5 | 100 | 11.4 | A |
| | 3-7 | R-7 | 105 | 32 | 17 | 2.5 | 120 | 11.4 | A |
| | 3-8 | R-8 | 100 | 34 | 16 | 2.5 | 110 | 10.8 | A |
| | 3-9 | R-9 | 100 | 33 | 17 | 2.4 | 100 | 11.2 | A |
| | 3-10 | R-10 | 100 | 34 | 18 | 2.4 | 120 | 11.1 | A |
| | 3-11 | R-11 | 105 | 33 | 17 | 2.4 | 100 | 10.9 | A |
| | 3-12 | R-12 | 100 | 34 | 17 | 2.5 | 110 | 11.0 | A |
| | 3-13 | R-13 | 95 | 33 | 16 | 2.6 | 120 | 10.8 | A |
| | 3-14 | R-14 | 100 | 33 | 18 | 2.4 | 110 | 10.8 | A |
| | 3-15 | R-15 | 100 | 35 | 16 | 2.3 | 100 | 11.3 | A |
| | 3-16 | R-16 | 100 | 34 | 17 | 2.3 | 110 | 11.1 | A |
| | 3-17 | R-17 | 100 | 34 | 16 | 2.4 | 120 | 11.1 | A |
| | 3-18 | R-18 | 100 | 34 | 18 | 2.6 | 110 | 11.0 | A |
| | 3-19 | R-19 | 100 | 33 | 16 | 2.3 | 120 | 11.5 | A |
| | 3-20 | R-20 | 100 | 32 | 17 | 2.5 | 100 | 11.3 | A |
| | 3-21 | R-21 | 100 | 33 | 16 | 2.5 | 120 | 11.3 | A |
| | 3-22 | R-22 | 105 | 35 | 17 | 2.4 | 100 | 11.2 | A |
| | 3-23 | R-23 | 100 | 34 | 18 | 2.5 | 110 | 11.4 | A |
| | 3-24 | R-24 | 100 | 34 | 16 | 2.6 | 120 | 11.2 | A |
| | 3-25 | R-25 | 105 | 33 | 17 | 2.5 | 120 | 11.1 | A |
| | 3-26 | R-26 | 100 | 35 | 16 | 2.4 | 110 | 10.8 | A |
| | 3-27 | R-27 | 95 | 33 | 18 | 2.3 | 100 | 10.8 | A |
| | 3-28 | R-28 | 105 | 35 | 17 | 2.4 | 120 | 11.4 | A |
| | 3-29 | R-29 | 100 | 34 | 18 | 2.5 | 100 | 11.1 | A |
| | 3-30 | R-30 | 95 | 35 | 18 | 2.4 | 110 | 11.3 | A |

TABLE 4

| | | | | Optimum | | | | | |
|---|---|---|---|---|---|---|---|---|---|
| | | | PEB | exposure | | | | Collapse | |
| | | Resist | temp. | dose | EL | LWR | DOF | limit | Development |
| | | composition | (° C.) | (mJ/cm²) | (%) | (nm) | (nm) | (nm) | defects |
| Comparative | 2-1 | CR-1 | 100 | 38 | 12 | 3.3 | 60 | 14.3 | B |
| Example | 2-2 | CR-2 | 100 | 37 | 13 | 2.8 | 80 | 12.2 | C |
| | 2-3 | CR-3 | 105 | 38 | 14 | 3.1 | 70 | 12.4 | B |
| | 2-4 | CR-4 | 100 | 39 | 14 | 2.9 | 80 | 12.3 | C |
| | 2-5 | CR-5 | 100 | 39 | 12 | 2.8 | 80 | 13.1 | B |
| | 2-6 | CR-6 | 100 | 42 | 14 | 3.2 | 80 | 13.1 | C |
| | 2-7 | CR-7 | 100 | 41 | 14 | 3.0 | 90 | 12.8 | C |
| | 2-8 | CR-8 | 100 | 40 | 13 | 2.9 | 70 | 12.9 | B |
| | 2-9 | CR-9 | 105 | 38 | 14 | 3.1 | 70 | 13.1 | C |
| | 2-10 | CR-10 | 100 | 38 | 12 | 3.3 | 80 | 13.0 | B |
| | 2-11 | CR-11 | 100 | 38 | 14 | 3.0 | 90 | 13.1 | B |
| | 2-12 | CR-12 | 100 | 38 | 14 | 2.7 | 80 | 13.2 | C |
| | 2-13 | CR-13 | 100 | 37 | 13 | 3.0 | 90 | 12.6 | B |
| | 2-14 | CR-14 | 95 | 40 | 13 | 2.7 | 70 | 12.4 | C |
| | 2-15 | CR-15 | 105 | 38 | 14 | 3.1 | 100 | 13.1 | B |
| | 2-16 | CR-16 | 95 | 39 | 13 | 3.2 | 70 | 13.1 | C |
| | 2-17 | CR-17 | 100 | 39 | 14 | 2.7 | 90 | 12.6 | B |
| | 2-18 | CR-18 | 100 | 38 | 14 | 3.1 | 70 | 12.2 | C |
| | 2-19 | CR-19 | 100 | 40 | 13 | 3.2 | 70 | 12.5 | B |
| | 2-20 | CR-20 | 100 | 39 | 14 | 2.8 | 80 | 13.2 | B |

It is demonstrated in Tables 3 and 4 that chemically amplified resist compositions comprising the photoacid generators within the scope of the invention exhibit a high sensitivity and improved values of EL, LWR and DOF. Small values of collapse limit attest that in forming a small-size pattern, the pattern is resistant to collapse. Furthermore, it is demonstrated that development defects were also suppressed. The chemically amplified resist compositions of the invention are suitable as materials for EUV lithography.

### [5]EUV Lithography Test (2)

### Examples 4-1 to 4-30, Comparative Examples 3-1 to 3-20

Each of the chemically amplified resist compositions (R-1 to R-30, CR-1 to CR-20) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, σ 0.9/0.6, quadrupole illumination), the resist film was exposed to EUV through a mask bearing a hole pattern having a pitch of 46 nm +20% bias (on-wafer size). The resist film was baked (PEB) on a hotplate at the temperature shown in Tables 5 and 6 for 60 seconds and developed in a 2.38 wt % TMAH aqueous solution for 30 seconds to form a hole pattern having a size of 23 nm.

The pattern as developed was observed under CD-SEM (CG6300, Hitachi High-Technologies Corp.). The dose (mJ/cm²) at which a pattern with a hole size of 23 nm was printed was determined as an index of sensitivity. The size of 50 holes was measured, from which a 3-fold value (3σ) of the standard deviation (σ) was determined as critical dimension uniformity (CDU). The results are shown in Tables 5 and 6.

TABLE 5

| | | | | Optimum | |
|---|---|---|---|---|---|
| | | | PEB | exposure | |
| | | Resist | temp. | dose | CDU |
| | | composition | (° C.) | (mJ/cm²) | (nm) |
| Example | 4-1 | R-1 | 90 | 24 | 2.4 |
| | 4-2 | R-2 | 90 | 25 | 2.4 |
| | 4-3 | R-3 | 95 | 23 | 2.4 |
| | 4-4 | R-4 | 90 | 24 | 2.6 |
| | 4-5 | R-5 | 90 | 24 | 2.4 |
| | 4-6 | R-6 | 90 | 24 | 2.6 |
| | 4-7 | R-7 | 95 | 23 | 2.5 |
| | 4-8 | R-8 | 95 | 23 | 2.4 |
| | 4-9 | R-9 | 90 | 25 | 2.3 |
| | 4-10 | R-10 | 95 | 26 | 2.4 |
| | 4-11 | R-11 | 95 | 23 | 2.6 |
| | 4-12 | R-12 | 90 | 24 | 2.5 |
| | 4-13 | R-13 | 90 | 23 | 2.4 |
| | 4-14 | R-14 | 95 | 24 | 2.4 |
| | 4-15 | R-15 | 95 | 25 | 2.6 |
| | 4-16 | R-16 | 85 | 23 | 2.3 |
| | 4-17 | R-17 | 95 | 24 | 2.5 |
| | 4-18 | R-18 | 90 | 25 | 2.5 |
| | 4-19 | R-19 | 95 | 25 | 2.4 |
| | 4-20 | R-20 | 90 | 24 | 2.5 |
| | 4-21 | R-21 | 90 | 24 | 2.4 |
| | 4-22 | R-22 | 90 | 25 | 2.6 |
| | 4-23 | R-23 | 95 | 24 | 2.5 |
| | 4-24 | R-24 | 90 | 24 | 2.4 |
| | 4-25 | R-25 | 90 | 25 | 2.5 |
| | 4-26 | R-26 | 85 | 23 | 2.4 |
| | 4-27 | R-27 | 95 | 23 | 2.3 |
| | 4-28 | R-28 | 90 | 25 | 2.6 |
| | 4-29 | R-29 | 90 | 26 | 2.3 |
| | 4-30 | R-30 | 95 | 24 | 2.4 |

TABLE 6

| | | | | Optimum | |
|---|---|---|---|---|---|
| | | | PEB | exposure | |
| | | Resist | temp. | dose | CDU |
| | | composition | (° C.) | (mJ/cm²) | (nm) |
| Comparative | 3-1 | CR-1 | 90 | 31 | 3.2 |
| Example | 3-2 | CR-2 | 90 | 29 | 2.8 |
| | 3-3 | CR-3 | 95 | 28 | 2.7 |
| | 3-4 | CR-4 | 90 | 29 | 2.7 |
| | 3-5 | CR-5 | 95 | 30 | 3.0 |
| | 3-6 | CR-6 | 85 | 31 | 3.1 |
| | 3-7 | CR-7 | 90 | 28 | 2.9 |
| | 3-8 | CR-8 | 90 | 27 | 2.8 |
| | 3-9 | CR-9 | 90 | 30 | 2.7 |
| | 3-10 | CR-10 | 95 | 29 | 2.8 |
| | 3-11 | CR-11 | 90 | 27 | 3.0 |
| | 3-12 | CR-12 | 90 | 28 | 2.8 |
| | 3-13 | CR-13 | 95 | 29 | 2.7 |
| | 3-14 | CR-14 | 90 | 27 | 3.1 |
| | 3-15 | CR-15 | 90 | 29 | 2.8 |
| | 3-16 | CR-16 | 90 | 29 | 3.0 |
| | 3-17 | CR-17 | 95 | 27 | 3.0 |
| | 3-18 | CR-18 | 90 | 29 | 2.7 |
| | 3-19 | CR-19 | 85 | 30 | 2.8 |
| | 3-20 | CR-20 | 90 | 31 | 2.8 |

It is demonstrated in Tables 5 and 6 that chemically amplified resist compositions within the scope of the invention exhibit a high sensitivity and satisfactory CDU.

Japanese Patent Application No. 2024-132003 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. An onium salt consisting of a cation having the formula (1A) and an anion having the formula (1B): 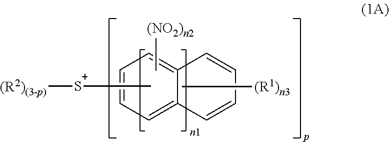
wherein p is 1, 2, or 3, n1 is 0 or 1, n2 is 1 or 2, n3 is 0, 1, 2, or 3, meeting 1≤n2+n3≤5 in case of n1=0 and 1 n2+n3 7 in case of n1=1,
R¹ is halogen, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, and when n3 is 2 or 3, a plurality of R¹ may be identical or different and two R¹ may bond together to form a ring with the carbon atoms to which they are attached,
R² is halogen or a C₁-C₃₀ hydrocarbyl group which may contain a heteroatom, when p is 1, two R² may be identical or different, and any two of three substituents bonded to S⁺ may bond together to form a ring with the sulfur atoms to which they are attached; 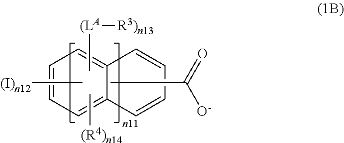
wherein n11 is 0 or 1, n12 is 1, 2, 3, or 4, n13 is 0, 1, or 2, n14 is 0, 1, 2, 3, or 4, meeting 2≤n12+n13+n14≤5 in case of n11=0 and 2≤n12+n13+n14≤7 in case of n11=1,
L^{A} is a single bond, oxygen, sulfur, ester bond or carbonate bond,
R³ is hydrogen or a C₁-C₂₀ hydrocarbyl group which may contain a heteroatom when L^{A} is a single bond, and hydrogen, a C₁-C₂₀ hydrocarbyl group (exclusive of acid labile group) which may contain a heteroatom, or acid labile group when L^{A} is oxygen, sulfur, ester bond or carbonate bond, and
R⁴ is halogen exclusive of iodine, a nitro group, cyano group, C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom.

2. The onium salt of claim 1 wherein the cation has the formula (1A-1): 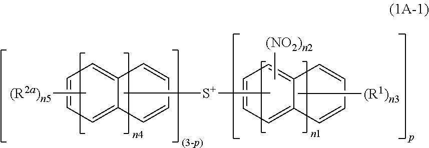
wherein p, n1 to n3, R¹, and Z⁻ are as defined above,
n4 is 0 or 1, n5 is 0, 1, 2, 3, 4, or 5, and
R^{2a} is halogen, cyano, hydroxy, carboxy, alkoxycarbonyl, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, and when n5 is 2, 3, 4, or 5, a plurality of R^{2a} may be identical or different and two R^{2a} may bond together to form a ring with the carbon atoms to which they are attached.

3. The onium salt of claim 1 wherein the anion has the formula (1B-1): 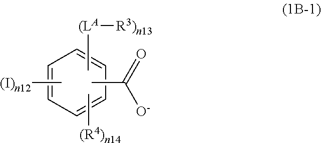
wherein n12 to n14, L^{A}, R³ and R⁴ are as defined above.

4. The onium salt of claim 1 wherein L^{A} is oxygen or ester bond.

5. The onium salt of claim 1 wherein L^{A} is oxygen, sulfur, ester bond or carbonate bond and R³ is an acid labile group.

6. The onium salt of claim 1 wherein the acid labile group has the formula (AL-1) or (AL-2): 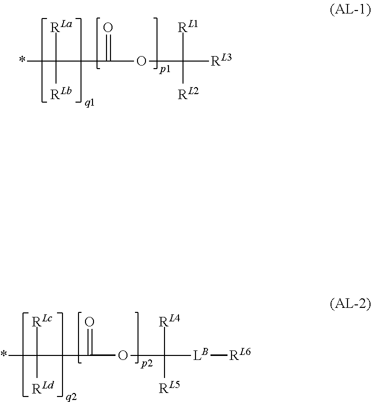
wherein p1 and p2 are each independently 0 or 1, q1 and q2 are each independently 0, 1, 2, 3, or 4,
R^{L1}, R^{L2} and R^{L3} are each independently a C₁-C₁₂ hydrocarbyl group in which some —CH₂— may be replaced by —O— or —S—, when the hydrocarbyl group contains an aromatic ring, some or all of the hydrogen atoms in the aromatic ring may be substituted by halogen, cyano moiety, nitro moiety, optionally halogenated C₁-C₄ alkyl moiety, or optionally halogenated C₁-C₄ alkoxy moiety, R^{L1} and R^{L2} may bond together to form a ring with the carbon atom to which they are attached, some —CH₂— in the ring may be replaced by —O— or —S—,
R^{L4} and R^{L5} are each independently hydrogen or a C₁-C₁₀ hydrocarbyl group, R^{L6} is a C₁-C₂₀ hydrocarbyl group in which some —CH₂— may be replaced by —O— or —S—, R^{L5} and R^{L6} may bond together to form a C₃-C₂₀ heterocyclic group with the carbon atom and L^{B} to which they are attached, some —CH₂— in the heterocyclic group may be replaced by —O— or —S—,
L^{B} is —O— or —S—,
R^{La} to R^{Ld} are each independently hydrogen or a C₁-C₂₀ hydrocarbyl group which may contain a heteroatom, and
the asterisk (*) designates a point of attachment to the adjacent oxygen or sulfur.

7. A quencher in the form of the onium salt of claim 1.

8. A chemically amplified resist composition comprising the quencher of claim 7.

9. The chemically amplified resist composition of claim 8, comprising a base polymer comprising repeat units having the formula (a1): 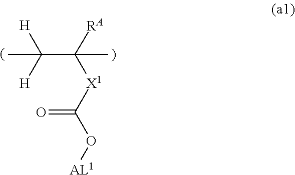
wherein R^{A} is hydrogen, fluorine, methyl or trifluoromethyl,
X¹ is a single bond, phenylene group, naphthylene group, *—C(═O)—O—X¹¹— or *—C(═O)—NH—X¹¹—, the phenylene or naphthylene group may be substituted with an optionally fluorinated C₁-C₁₀ alkoxy moiety or halogen, X¹¹ is a C₁-C₁₀ saturated hydrocarbylene group which may contain a hydroxy moiety, ether bond, ester bond or lactone ring, or phenylene or naphthylene group, the asterisk (*) designates a point of attachment to the carbon atom in the backbone, and
AL¹ is an acid labile group.

10. The chemically amplified resist composition of claim 9 wherein the base polymer comprises repeat units having the formula (a2): 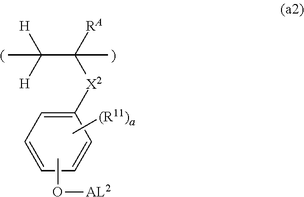
wherein R^{A} is hydrogen, fluorine, methyl or trifluoromethyl,
X² is a single bond or *—C(═O)—O—, the asterisk (*) designates a point of attachment to the carbon atom in the backbone,
R¹¹ is halogen, cyano, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R¹¹ may be identical or different when a1 is 2, 3, or 4,
AL² is an acid labile group, and
a is 0, 1, 2, 3, or 4.

11. The chemically amplified resist composition of claim 9 wherein the base polymer comprises repeat units having the formula (a3): 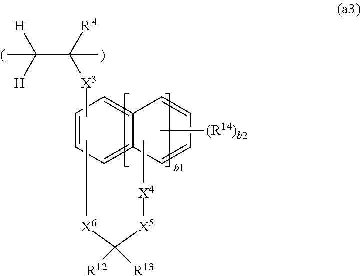
wherein b1 is 0 or 1, b2 is 0, 1, 2, or 3 in case of b1=0 and 0, 1, 2, 3, 4, or 5 in case of b1=1,
R^{A} is hydrogen, fluorine, methyl or trifluoromethyl,
X³ is a single bond, *—C(═O)—O— or *—C(═O)—NH—, the asterisk (*) designates a point of attachment to the carbon atom in the backbone,
R¹² and R¹³ are each independently hydrogen or a C₁-C₂₀ hydrocarbyl group which may contain a heteroatom, R¹² and R¹³ may bond together to form a ring with the carbon atom to which they are attached,
R¹⁴ is halogen, hydroxy, cyano, nitro, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group, a plurality of R¹⁴ may bond together to form a ring with the carbon atoms on the aromatic ring to which they are attached when b2 is 2 or more,
X⁴ is a single bond, C₁-C₄ aliphatic hydrocarbylene group, carbonyl group, sulfonyl group, or a group obtained by combining the foregoing,
X⁵ and X⁶ are each independently oxygen or sulfur, X⁴ and X⁶ are bonded to vicinal carbon atoms on the aromatic ring.

12. The chemically amplified resist composition of claim 9 wherein the base polymer comprises repeat units having the formula (bi) or (b2): 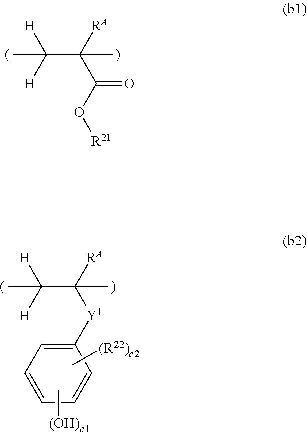
wherein R^{A} is each independently hydrogen, fluorine, methyl or trifluoromethyl,
Y¹ is a single bond or *—C(═O)—O—, the asterisk (*) designates a point of attachment to the carbon atom in the backbone,
R²¹ is hydrogen or a C₁-C₂₀ group containing at least one structure selected from among hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—),
R²² is halogen, carboxy, nitro, cyano, alkoxycarbonyl, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R²² may be identical or different when c2 is 2, 3, or 4, and
c1 is 1, 2, 3, or 4 and c2 is 0, 1, 2, 3, or 4, meeting 1≤c1+c2≤5.

13. The chemically amplified resist composition of claim 9 wherein the base polymer comprises at least one selected from repeat units having the formula (c1), repeat units having the formula (c2), repeat units having the formula (c3), repeat units having the formula (c4), and repeat units having the formula (c5): 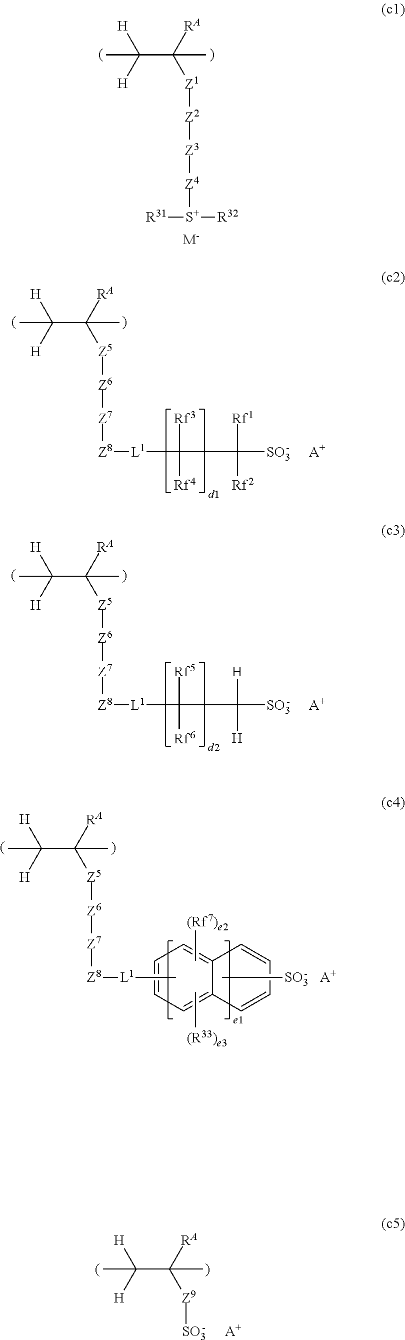
wherein d1 and d2 are each independently 0, 1, 2, or 3, e1 is 0 or 1, e2 is 0, 1, 2, 3, or 4, e3 is 0, 1, 2, 3, or 4, meeting 0≤e2+e3≤4 in case of e1=0 and 0≤e2+e3≤6 in case of e1=1,
R^{A} is each independently hydrogen, fluorine, methyl or trifluoromethyl,
Z¹ is a single bond or optionally substituted phenylene group,
Z² is a single bond, **—C(═O)—O—Z²¹—, **—C(═O)—NH—Z²¹—, or **—O—Z²¹—, Z²¹ is a C₁-C₆ aliphatic hydrocarbylene group, phenylene group or a divalent group obtained by combining the foregoing, which may contain halogen, a carbonyl moiety, ester bond, ether bond or hydroxy moiety,
Z³ is a single bond, ether bond, ester bond, amide bond, sulfonate ester bond, sulfonamide bond, carbonate bond or carbamate bond,
Z⁴ is a single bond, or a C₁-C₆ aliphatic hydrocarbylene group, phenylene group or a divalent group obtained by combining the foregoing, which may contain halogen, carbonyl moiety, ester bond, ether bond or hydroxy moiety,
Z⁵ is each independently a single bond, optionally substituted phenylene group, naphthylene group, or *—C(═O)—O—Z⁵, Z⁵¹ is a C₁-C₁₀ aliphatic hydrocarbylene group which may contain halogen, hydroxy moiety, ether bond, ester bond or lactone ring, or phenylene or naphthylene group,
Z⁶ is a single bond, ether bond, ester bond, amide bond, sulfonate ester bond, sulfonamide bond, carbonate bond or carbamate bond,
Z⁷ is each independently a single bond, ***—Z⁷¹—C(═O)—O—, ***—C(═O)—NH—Z⁷¹—, or ***—O—Z⁷¹—, Z⁷¹ is a C₁-C₂₀ hydrocarbylene group which may contain a heteroatom,
Z⁸ is each independently a single bond, ****—Z⁸¹—C(═O)—O—, ****—C(═O)—NH—Z⁸¹—, or ****—O—Z⁸¹—, Z⁸¹ is a C₁-C₂₀ hydrocarbylene group which may contain a heteroatom,
Z⁹ is a single bond, methylene, ethylene, phenylene, fluorinated phenylene, trifluoromethyl-substituted phenylene, *—C(═O)—O—Z⁹¹—, *—C(═O)—N(H)—Z⁹¹—, or *—O—Z⁹¹—, Z⁹¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety,
the asterisk (*) designates a point of attachment to the carbon atom in the backbone, the asterisks (**) designate a point of attachment to Z¹, the asterisks (***) designate a point of attachment to Z⁶, the asterisks (****) designate a point of attachment to Z⁷,
L¹ is a single bond, ether bond, ester bond, carbonyl group, sulfonate ester bond, sulfonamide bond, carbonate bond or carbamate bond,
Rf¹ and Rf² are each independently fluorine or a C1-C₆ fluorinated saturated hydrocarbyl group,
Rf³ and Rf⁴ are each independently hydrogen, fluorine, or a C₁-C₆ fluorinated saturated hydrocarbyl group,
Rf⁵ and Rf⁶ are each independently hydrogen, fluorine, or a C₁-C₆ fluorinated saturated hydrocarbyl group, excluding that all Rf⁵ and Rf^{P} are hydrogen at the same time,
Rf⁷ is fluorine, a C₁-C₆ fluorinated alkyl group, a C₁-C₆ fluorinated alkoxy group, or a C₁-C₆ fluorinated alkylthio group,
R³¹ and R³² are each independently a C₁-C₂₀ hydrocarbyl group which may contain a heteroatom, R³¹ and R³² may bond together to form a ring with the sulfur atom to which they are attached,
R³³ is halogen exclusive of fluorine, or a C₁-C₂₀ hydrocarbyl group which may contain a heteroatom, when e3 is 2, 3, or 4, a plurality of R³³ may be identical or different and a plurality of R³³ may bond together to form a ring with the carbon atoms to which they are attached,
M⁻ is a non-nucleophilic counter ion, and
A⁺ is an onium cation.

14. The chemically amplified resist composition of claim 8, further comprising an organic solvent.

15. The chemically amplified resist composition of claim 8, further comprising a photoacid generator capable of generating a strong acid.

16. The chemically amplified resist composition of claim 8, further comprising another quencher other than the quencher.

17. The chemically amplified resist composition of claim 8, further comprising a surfactant.

18. A pattern forming process comprising the steps of applying the chemically amplified resist composition of claim 8 onto a substrate to form a resist film thereon, exposing the resist film to high-energy radiation, and developing the exposed resist film in a developer.

19. The pattern forming process of claim 18 wherein the high-energy radiation is KrF excimer laser, ArF excimer laser, electron beam, or extreme ultraviolet of wavelength 3 to 15 nm.
